(19) United States
(12) Patent Application Publication
(10) Pub. No.: US 2023/0286970 A1
(43) Pub. Date: Sep. 14, 2023

(54) NOVEL OXADIAZOLE-BASED SELECTIVE HDAC6 INHIBITORS

(71) Applicant: ITALFARMACO S.P.A., Milano (IT)

(72) Inventors: Mattia MARCHINI, Milano (IT); Barbara VERGANI, Macherio (IT); Giovanni SANDRONE, Novara (IT); Ilaria ROCCHIO, Sesto San Giovanni (IT); Georgii KACHKOVSKYI, Cracow (PL); Gianluca CAPRINI, Somma Lombardo (IT); Gianluca FOSSATI, Milano (IT); Christian STEINKÜHLER, Roma (IT); Andrea STEVENAZZI, Milano (IT)

(21) Appl. No.: 18/040,708

(22) PCT Filed: Jul. 30, 2021
(86) PCT No.: PCT/EP2021/071465

§ 371 Date: Feb. 6, 2023

(30) Foreign Application Priority Data
Aug. 7, 2020 (IT) 102020000019714

(51) Int. Cl. C07D 413/14; C07D 413/10; C07D 487/04; C07D 401/14; C07D 495/04; C07D 471/10; C07D 487/10; C07D 417/14

(52) U.S. Cl. CPC C07D 413/14; C07D 413/10; C07D 487/04; C07D 401/14; C07D 495/04; C07D 471/10; C07D 487/10; C07D 417/14

(57) ABSTRACT

The present invention relates to novel selective oxadiazole-based inhibitors of histone deacetylase 6 (HDAC6) bearing a pentaheterocyclic scaffold and pharmaceutical compositions thereof.

### FIELD OF THE INVENTION

The present invention relates to novel selective oxadiazole-based inhibitors of histone deacetylase 6 (HDAC6) bearing a pentaheterocyclic scaffold and pharmaceutical compositions thereof.

Therefore, these compounds are useful in treating diseases associated with HDAC6 activity such as peripheral neuropathy, graft rejection, GVHD, myositis, diseases associated with abnormal lymphocyte function, multiple myeloma, non-Hodgkin lymphoma, autoimmune diseases, inflammatory diseases, cancer and neurodegenerative pathologies.

### STATE OF THE ART OF THE INVENTION

The genetic material of eukaryotic cells is organized in a complex and dynamic structure consisting of DNA and proteins, chromatin. The main protein components of chromatin are histones, basic proteins which interact with DNA forming the basic structural unit of chromatin, the nucleosome, the first level of chromosomal compaction within nucleus. The interaction between basic histone residues and DNA acid residues is crucial in determining the nucleosome compaction and the related DNA accessibility to molecular complexes regulating replication and transcription. This interaction is mainly influenced by histone degree of acetylation. Deacetylation of histone N-terminal lysine residues enables protonation of amine group, which carrying a positive charge, interacts with negative charges contained in DNA. Such interaction occurs in a more compact state of chromatin, involving the gene expression silencing. Conversely, acetylation of the same residues prevents ionic bonding formation, leading to a less compact form of chromatin which allows greater DNA exposure and the interaction with macromolecular complexes that activate gene transcription.

The degree of histone acetylation is regulated by the activity balance of two classes of enzymes: histone acetyl transferases (histone acetyl-transferases HAT) and histone deacetylase (histone deacetylases HDAC). An alteration of this delicate balance can lead to a loss of cellular homeostasis, commonly found in various human diseases, including cancer, neurological disorders, inflammation, and autoimmune diseases. Histone deacetylases have been so classified as they reversibly catalyse the deacetylation of amine groups of histone N-terminus lysine residues. Subsequently, it has been found that there is a large number of substrates of these enzymes as their activity is also due to non-histone protein which are substrates of HAT enzymes containing N-acetyl-lysine, such as transcription factors, DNA repair enzymes and other nucleus and cytoplasmic proteins.

The human HDAC class consists of 18 enzymes, divided into two groups: zinc-dependent HDACs and HDAC NAD-dependent, also known as sirtuins (class III). Zinc-dependent HDACs are further distributed into four classes: 1) Class I, including HDAC1, 2, 3 and 8, ubiquitous isoenzymes mainly located in the nucleus; 2) Class IIa, including HDAC4, 5, 7 and 9, isoenzymes located both in the nucleus and the cytoplasm; 3) Class IIb, including HDAC6 and HDAC10, mainly located in the cytoplasm and 4) Class IV, including only HDAC11. Unlike Class I HDACs, Class IIa and IIb have a tissue-specific expression.

By regulating gene expression and acting on histones and transcription factors, these enzymes are involved in a myriad of cellular functions. In addition, by acting on numerous other protein substrates, these enzymes, as well as phosphatases, are involved in many other processes such as signal transduction and cytoskeleton rearrangement.

In the recent decades, HDACs have become a well-studied therapeutic target. Several HDAC inhibitors have been synthesized, some of which are currently in advanced clinical trials and four of them have been approved for different types of cancer: Vorinostat and Rom idepsin for Cutaneous T-cell lymphoma (CTLC), Belinostat for Cell Peripheral T-cell lymphoma (PTLC) and Panobinostat for multiple myeloma. These inhibitors can interact with different HDAC isoforms.

Despite their clinical efficacy, the use of pan-inhibitors, thus non-selective for a single isoform, is limited by their toxicity and side effects observed in both preclinical models and, most importantly, in clinical trials. Hence the need for developing HDAC inhibitors with a better pharmacological profile and therapeutic window (efficacy/toxicity ratio). The attention of the scientific community has thus focused on the synthesis and study of selective inhibitors for individual HDAC isoforms, aiming to develop molecules with better pharmacological capabilities.

Therefore, the use of HDAC inhibitors can be an important therapeutic or diagnostic tool for pathologies caused by gene expression such as inflammatory disorders, diabetes, diabetes complications, homozygous thalassemia, fibrosis, cirrhosis, acute promyelocytic leukaemia (APL), organ transplant rejection, autoimmune pathologies, protozoal infections, cancers, etc. Furthermore, alteration of HDAC activity has also been correlated to chemotherapy induced peripheral neuropathy (CIPN) and Charcot-Marie-Tooth disease (CMT), the most common inherited peripheral neuropathy. Selective inhibitors for a HDAC family or for a specific isoform, especially HDAC6, may be particularly useful for treating pathologies related to proliferative disorders and protein accumulation, immune system disorders and neurological and neurodegenerative disease, such as stroke, Huntington's disease, Amyotrophic Lateral Sclerosis (ALS), Alzheimer's disease, CIPN and CMT.

Especially for HDAC6, different substrates have been identified, such as α-tubulin, Hsp90 (Heat Shock Protein 90), cortactin, β-catenin. Modulation of the acetylation of these proteins by HDAC6 has been correlated with several important processes, such as immune response (Kozikowski, J. Med. Chem. (2012), 55, 639-651; Mol. Cell. Biol. (2011), 31(10), 2066-2078), regulation of microtubule dynamics, including cell migration, cell-cell interaction (Aldana-Masangkay et al., J. Biomed. Biotechnol. (2011), 2011, 875824), axonal transport and axonal regeneration (Rossaert and Van Den Bosch, Brain Research, 2020, 1733, 146692).

In addition, HDAC6 is involved in the process of catabolism of degraded proteins through the complex known as aggresome: HDAC6 is able to bind polyubiquitinated proteins and dynein, thus activating a kind of delivery of denatured proteins along the microtubules to the aggresome (Kawaguchi et al., Cell (2003) 115 (6), 727-738). Alteration of this HDAC6 cytoprotective activity has been correlated with various neurodegenerative pathologies such as Parkinson's disease (Outerio et al., Science (2007), 317 (5837), 516-519) and Huntington's disease (Dompierre et al., J. Neurosci. (2007), 27(13), 3571-3583), wherein the accumulation of degraded proteins is a common pathological feature.

HDAC6's involvement in microtubule dynamics and in elimination of misfolded proteins has been correlated to axonal transport deficits, commonly observed in peripheral neuropathy both genetically originated and chemotherapy induced. (Krukowski et al., Pain, 2017, 158(6), 1126-1137)

Further, HDAC6 is involved in regulating many oncological proteins, especially in hematologic tumours, such as various types of leukaemia (Fiskus et al., Blood (2008), 112(7), 2896-2905) and multiple myeloma (Hideshima et al., Proc. Natl. Acad. Sci. USA (2005), 102(24), 8567-8572). Regulation of α-tubulin acetylation by HDAC6 may be implicated in metastasis onset, wherein cellular motility plays an important role (Sakamoto et al., J. Biomed. Biotechnol. (2011), 2011, 875824).

Several selective HDAC6 inhibitors have been synthesized and studied in the last decade. Some of them are still under active preclinical development and two of them, namely Ricolinostat and Citarinostat, are currently under clinical investigation.

Most of the selective HDAC6 inhibitors belong to the hydroxamate based class. The hydroxamate group has the important function of binding the Zn++ ion in the enzyme active site. Nevertheless, some level of toxicity and genotoxicity is associated to this moiety, likely because of its capability of non-specific metal binding and its tendency to release hydroxylamine (Kozikowski, ChemMedChem. 2016 January; 11(1): 15-21).

Accordingly, the discovery of new classes of selective HDAC6 inhibitors can be useful for the treatment of all disorders and diseases mentioned above especially when the treatment is chronic.

### SUMMARY OF THE INVENTION

Some International patent applications (WO2020158762, WO2019027054, WO2017018803, WO2017065473 and WO2017023133) have disclosed 2-(difluoromethyl)-1,3,4-oxadiazole as an intrinsically HDAC6 selective zinc binding group (ZBG). Unexpectedly, the replacement of the hydroxamic moiety with the difluoromethyloxadiazole moiety to the class of inhibitors described in the WO2018189340 is not sufficient for a good HDAC6 inhibition.

WO2020212479 discloses oxadiazole compounds suitable as HDAC6 inhibitors. Processes for their preparation and their medical uses in treating HDAC6-related diseases or disorders are also disclosed.

Present inventors have synthesized a large number of compounds in order to identify the right pentaheterocyclic scaffolds and the right combination of substitutions that guarantee the potency against HDAC6 along with the selectivity over the other isoforms and the metabolic stability.

In fact, relative to the hydroxamate analogs, some sub-classes, such as 1,2,4-triazoles and 1,5-disubstituted tetrazoles need a very fine exploration in order to achieve the desired potency.

This invention discloses a new oxadiazole based class of metabolically stable, potent and selective non-hydroxamate based HDAC6 inhibitors that bear a pentaheterocyclic scaffold.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “halogen” refers herein to fluorine (F), chlorine (Cl), bromine (Br), or iodine (I).

The term “C₁-C₄ alkyl” refers herein to a branched or linear hydrocarbon containing 1 to 4 carbon atoms. Examples of C₁-C₄ alkyl groups include, but are not limited to, methyl, ethyl, n-propyl, isopropyl, n-butyl, sec-butyl, isobutyl, tert-butyl; preferably methyl, ethyl, n-propyl, isopropyl.

The term “aryl” refers herein to mono- and poly-carbocyclic aromatic ring systems (i), wherein individual carbocyclic rings in the poly-carbocyclic ring systems may be fused or attached to each other by a single bond. Suitable aryl groups include, but are not limited to, phenyl, naphthyl and biphenyl.

The term “aryloxy” refers herein to O-aryl group, wherein “aryl” is as defined above.

The term “alkoxy” refers herein to O-alkyl group, wherein “alkyl” is as defined above.

The term “thioalkoxy” refers herein to S-alkyl group, wherein “alkyl” is as defined above. A preferred thioalkoxy group is thioethoxy (—SEt) or thiomethoxy (—SMe), and even more preferably it is thiomethoxy. In a different embodiment, the thioalkoxy group refers to an alkyl group wherein one of the nonterminal hydrocarbon units of the alkyl chain is replaced by a sulfur atom. The term “halogenated” refers herein to halogen substitution, in other words, any of the above alkyl, alkoxy, thioalkoxy groups may be fully or partially substituted with a halogen atom. Preferably, the halogen atom is F or Cl, and more preferably it is F. A preferred particular halogenated substituent is the trifluoromethyl (—CF₃) group.

The term “cycloalkyl” refers herein to a saturated or unsaturated hydrocarbon ring, preferably having 4 to 10 carbon atoms. Examples of cycloalkyl include cyclopropyl, cyclobutyl, cyclopentyl, cyclohexyl, cycloheptyl and cyclooctyl.

The term “arylalkyl” refers herein to an aryl radical as defined herein, attached to an alkyl radical as defined herein. An example of arylalkyl is benzyl.

The term “heterocycle” refers herein to a 4-, 5-, 6-, 7- or 8-membered monocyclic ring which is saturated or unsaturated and consisting of carbon atoms and one or more heteroatoms selected from N, O and S, and wherein the nitrogen and sulphur heteroatoms may optionally be oxidized and the nitrogen heteroatom can be optionally quaternized. The heterocyclic ring may be attached to any heteroatom or carbon atom, provided that the attachment results in the creation of a stable structure. The term also includes any bicyclic system wherein any of the above heterocyclic rings is fused to an aryl or another heterocycle. When the heterocyclic ring is an aromatic heterocyclic ring, it can be defined as a “heteroaromatic ring”.

The term “unsaturated ring” refers herein to a partially or completely unsaturated ring. For example, an unsaturated C6 monocyclic ring refers to cyclohexene, cyclohexadiene and benzene.

The term “substituted” refers herein to mono- or poly-substitution with a defined (or undefined) substituent provided that this single or multiple substitution is chemically allowed.

The term “physiologically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference.

The term “pharmaceutically acceptable salts or derivatives thereof” herein refers to those salts or derivatives which possess the biological effectiveness and properties of the salified or derivatized compound and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate, hydrogen sulphate, citrate, maleate, fumarate, trifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, 2008, herein incorporated by reference. The pharmaceutically acceptable derivatives include the esters, the ethers and the N-oxides.

The terms “comprising”, “having”, “including” and “containing” are to be understood as open terms (meaning “including, but not limited to”) and are to be considered as a support also for terms such as “essentially consist of”, “essentially consisting of”, “consist of” or “consisting of”.

The terms “essentially consists of”, “essentially consisting of” are to be understood as semi-closed terms, meanings that no other ingredient affecting the novel characteristics of the invention is included (therefore optional excipients can be included).

The terms “consists of”, “consisting of” are to be understood as closed terms.

The term “isomers” refers to stereoisomers (or spatial isomers), i.e. diastereoisomers and enantiomers.

The term “prodrugs” refers to pharmacologically inactive derivatives, which can undergo in vivo metabolic transformation to afford an active compound included in the general formula of this invention. Many different prodrugs are known in the art (Prodrug approach: an effective solution to overcome side-effects, Patil S. J., Shirote P. J., International Journal of Medical and Pharmaceutical Sciences, 2011, 1-13; Carbamate Prodrug Concept for Hydroxamate HDAC Inhibitors, Jung, Manfred et al., ChemMedChem, 2011, 1193-1198).

### DESCRIPTION OF THE INVENTION

The inventors have experimentally found that this new class of compounds, characterized by the presence of 2-(difluoromethyl)-1,3,4-oxadiazole and by a pentaheterocyclic central core that includes—1,2,3-triazole, 1,2,4-triazole, 2,5-disubstituted tetrazole, 1,5-disubstituted tetrazole, imidazole, 1,3,4-oxadiazole, 1,2,4-oxadiazole, 1,3,4-thiadiazole, 1,4-disubstituted pyrazole, isoxazole—exhibits a high and selective inhibitory activity against the HDAC6 enzyme. The pentaheterocyclic central core excludes the 1,3-disubstituted pyrazole and, as regards 1,2,3-triazole with the aryl-CHF₂-oxadiazole substituent on carbon atom and -LR² substituent on nitrogen atom (referring to formula I, B═C and M=N), a very fine exploration is needed in order to achieve a good potency.

In this connection, only compounds with an H-donor group in R² substituent showed a HDAC6 IC₅₀ lower than 700 nM.

Among the above scaffolds, 1,2,3-triazoles and 2,5-disubstituted tetrazoles show good potency regardless of the nature of X, X′, Y and Y′ of formula (I), whereas 1,2,4-triazoles and 1,5-disubstituted tetrazoles achieve high inhibition provided that the Markush structure of formula (I) is narrowed as follows:
Y and Y′ must be CH, X and X′ must be independently CF or CH, Z must be —S—, and R1 must be —CH₃ for the 1,2,4-triazole scaffold,
Y and Y′ must be CH, X and X′ can be independently CH or N, but not CF for the 1,5-disubstituted tetrazole scaffold.

Compounds in the present invention showed very low cytotoxicity, which made them suitable for a chronic use.

According to a first aspect, the present invention relates to compounds of formula (I) and pharmaceutically acceptable salts, isomers and prodrugs thereof:

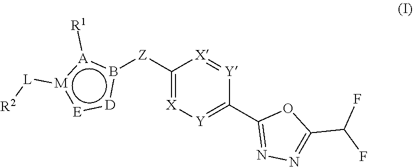

wherein:
X and X′ are independently selected from CH, N, CF or CCl;
Y and Y′ are independently selected from CH, N or CF;

### A=C, N, O, S;

### B═C, N;

### D=C, N, O;

### E=C, N, O;

### M=C, N;

Z═—CD₂-, —CF₂—, —CHR³—, —NH—, —S—;
R³═H, C₁-C₄ alkyl or can be selected among the following substructures:

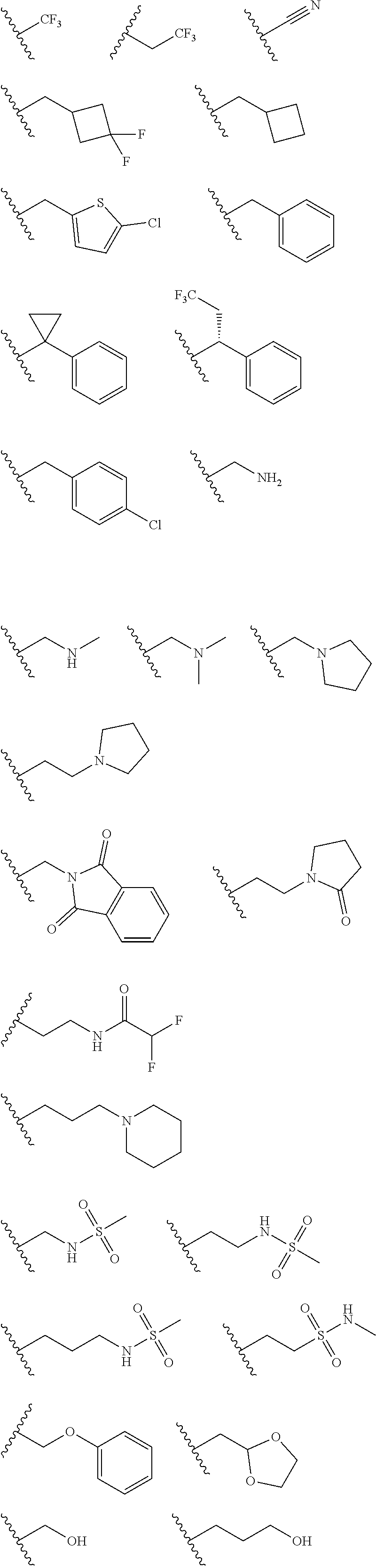

L=absent, C₁-C₄ alkyl, —CHPh-, —CH₂NHCH₂—, or can be selected among the following substructures:

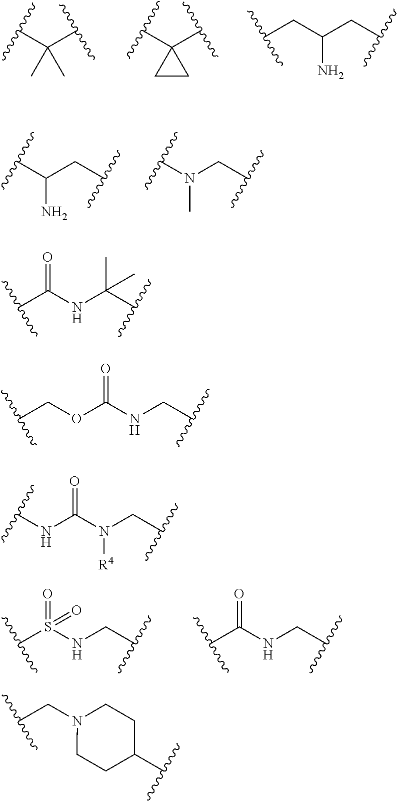

R⁴═H, C₁-C₄ alkyl;
R¹=absent, —H, C₁-C₄ alkyl, -LR². When R¹=-LR², substitution on M is absent;
R² is selected from the group consisting of:

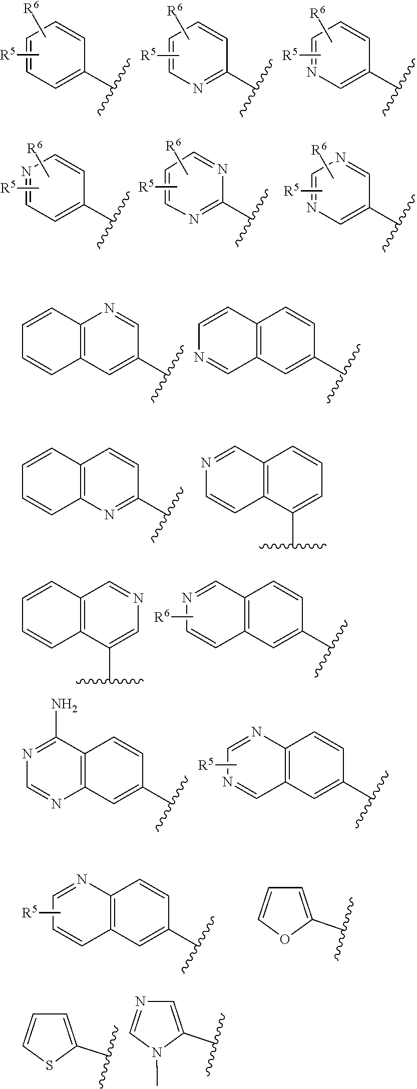

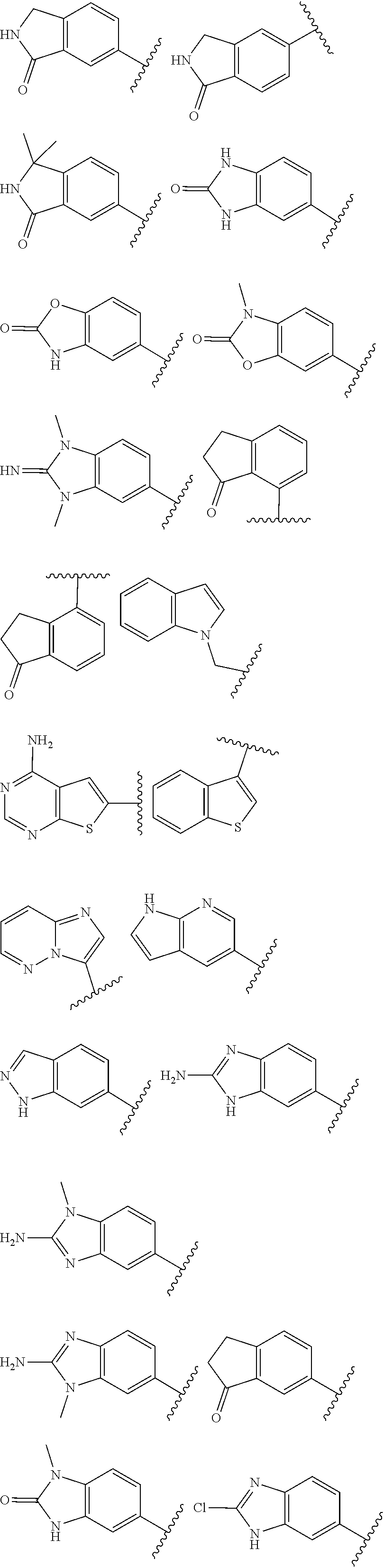

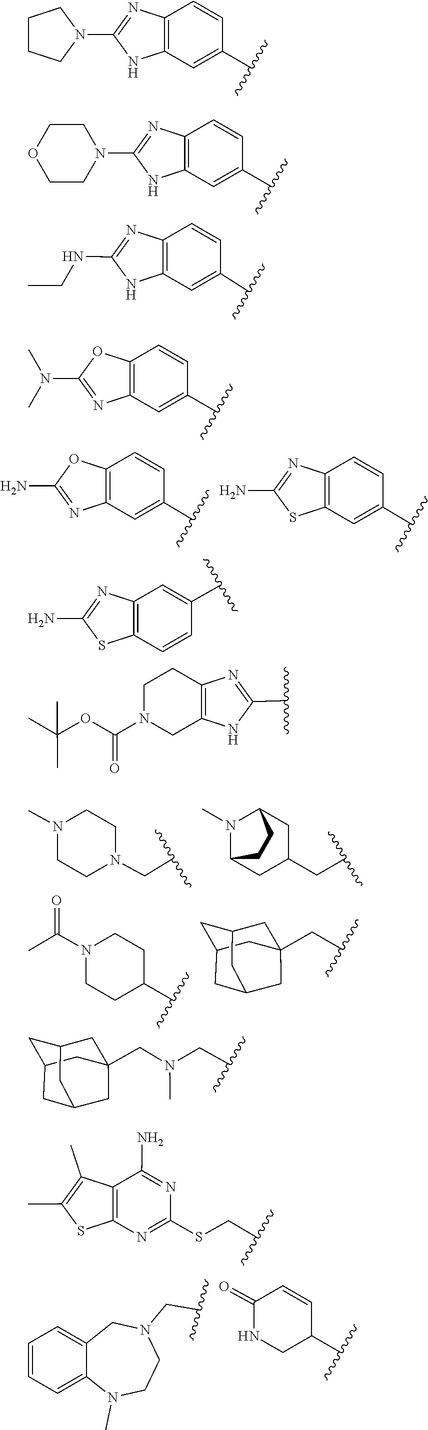

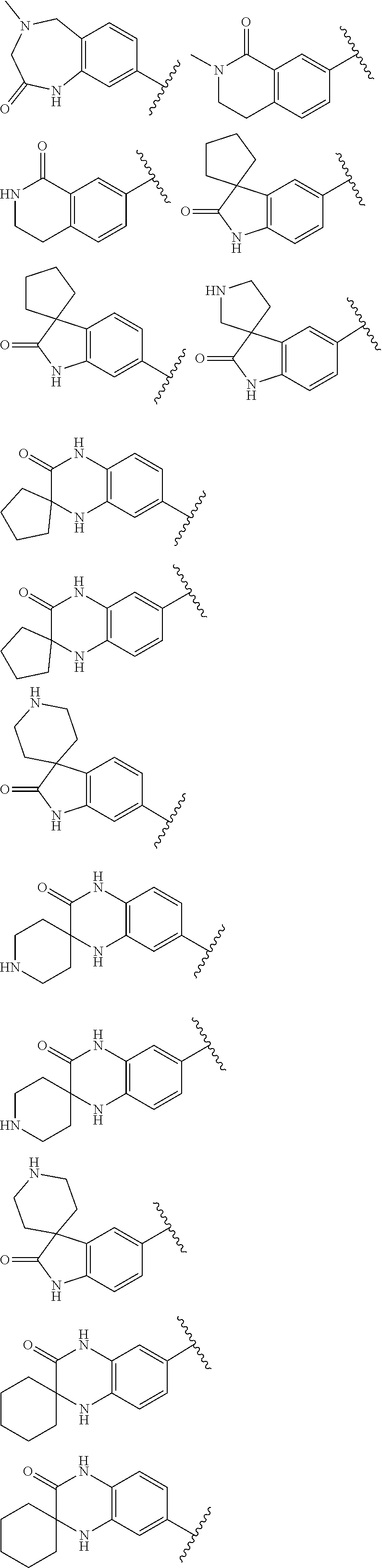

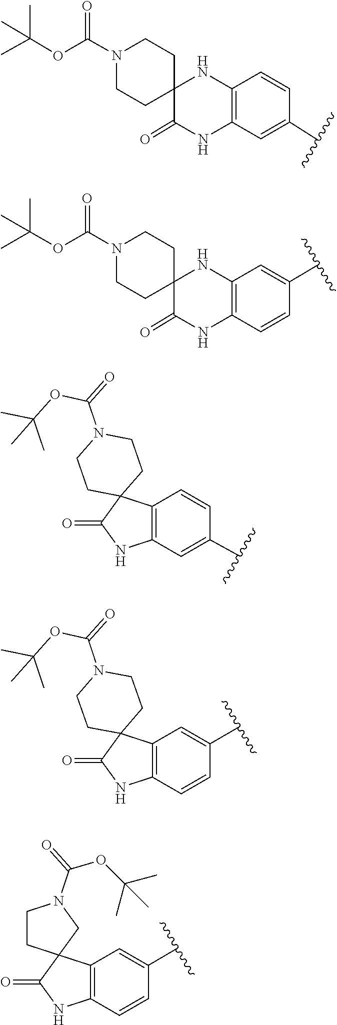 [SMILES: *c1ccc2c(c1)NC(=O)C1(CCN(C(=O)OC(C)(C)C)CC1)N2 ; *c1ccc2c(c1)NC1(CCN(C(=O)OC(C)(C)C)CC1)C(=O)N2 ; *c1ccc2c(c1)NC(=O)C21CCN(C(=O)OC(C)(C)C)CC1 ; *c1ccc2c(c1)C1(CCN(C(=O)OC(C)(C)C)CC1)C(=O)N2 ; *c1ccc2c(c1)C1(CCN(C(=O)OC(C)(C)C)C1)C(=O)N2]

R⁵ and R⁶ are independently selected from the group comprising: —H, -D, —OH, —O—C₁-C₄ alkyl, C₁-C₄ alkyl, -halogen, —CF₃, —NR′R″, —NHR⁷, —COOH, —COR⁸, —NO₂, —CN, -Ph, —SO₂NMe₂, —CH₂NH₂, or can be selected among the following substructures:

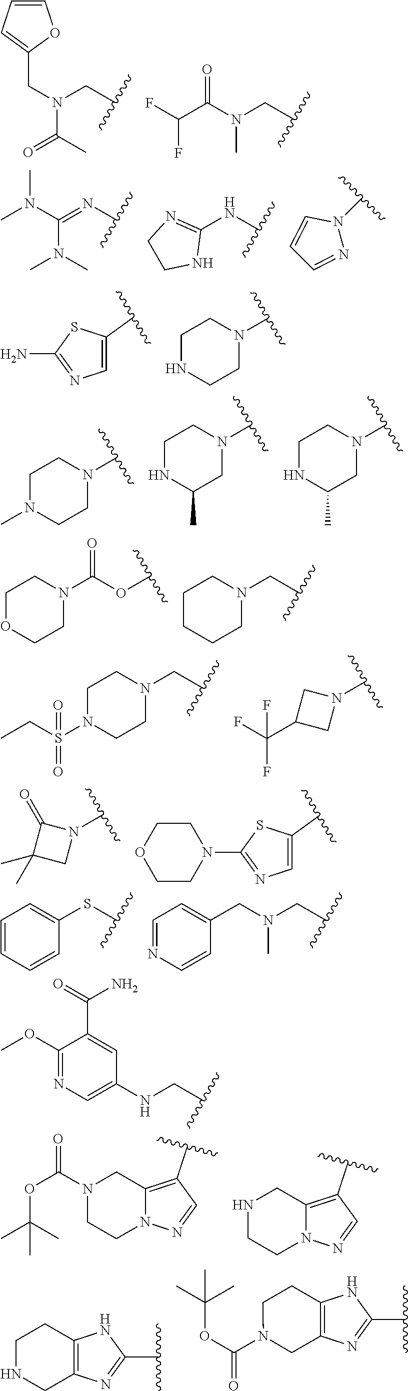

R⁷═—CH₂Ph, or can be selected among the following substructures:

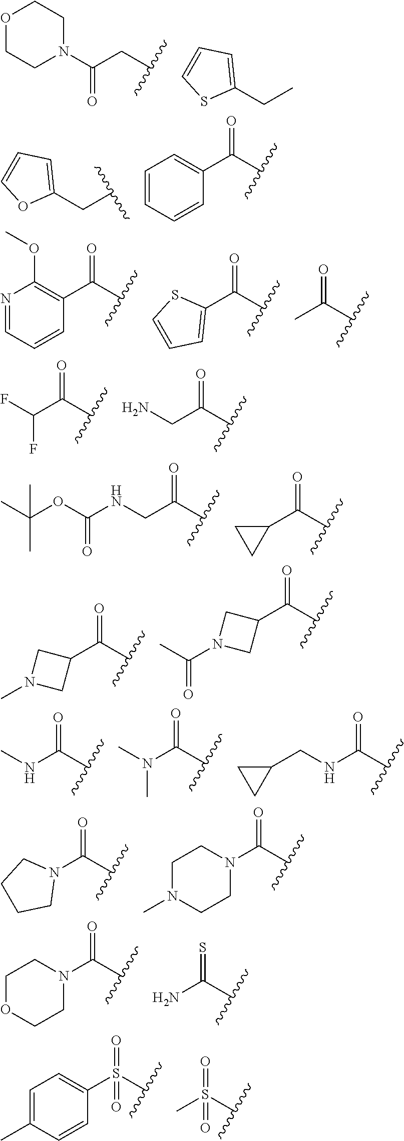

R⁸═—NR′R″, C₁-C₄ alkyl or can be selected among the following substructures:

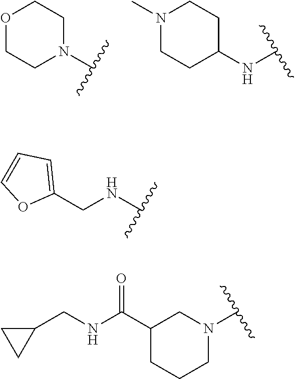

wherein R′ and R″ are independently —H or C₁-C₄ alkyl;
with the proviso that:
when A, D and E=N, B and M=C, (i.e., when the central heterocycle is 1,2,4-triazole), then Y and Y′═CH; X and X′ are independently selected from CH or CF; Z═—S—; R₁=Me;
when A=C and B, D, E and M=N (i.e., when the central heterocycle is 1,5-disubstituted tetrazole), then Y and Y′═CH; X and X′ are independently selected from CH or N; R₁=-LR².

Preferably, when A, D and E=N, B and M=C and when A=C and B, D, E and M=N (i.e., when the central heterocycle is 1,2,4-triazole or 1,5-disubstituted tetrazole), then R² is selected from the following substructures:

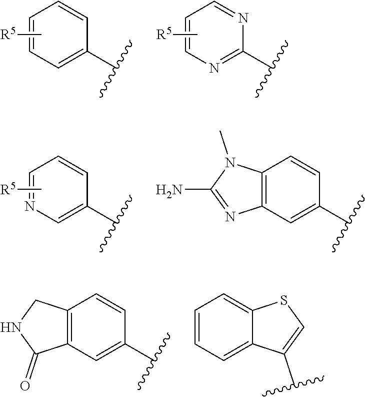

wherein:
R⁵═—NH₂, or is selected among the following substructures:

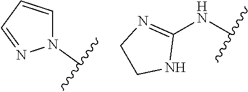

The following compounds of formula (I) are preferred:
6-(1-((5-(5-(difluoromethyl)-1,3,4-oxadiazol-2-yl)pyridin-2-yl)methyl)-1H-1,2,3-triazol-4-yl)benzo[d]thiazol-2-amine (compd. 1);
N-(5-(2-((5-(5-(difluoromethyl)-1,3,4-oxadiazol-2-yl)pyridin-2-yl)methyl)-2H-tetrazol-5-yl)-2-hydroxyphenyl)morpholine-4-carboxamide (compd. 2);
5-(1-((5-(5-(difluoromethyl)-1,3,4-oxadiazol-2-yl)pyridin-2-yl)methyl)-1H-1,2,3-triazol-4-yl)benzo[d]thiazol-2-amine (compd. 3);
6-(2-((5-(5-(difluoromethyl)-1,3,4-oxadiazol-2-yl)pyridin-2-yl)methyl)-2H-tetrazol-5-yl)isoindolin-1-one (compd. 4);
5-(1-((5-(5-(difluoromethyl)-1,3,4-oxadiazol-2-yl)pyridin-2-yl)methyl)-1H-1,2,3-triazol-4-yl)pyridin-2-amine (compd. 5);
N-(3-(1-((5-(5-(difluoromethyl)-1,3,4-oxadiazol-2-yl)pyridin-2-yl)methyl)-1H-1,2,3-triazol-4-yl)phenyl)morpholine-4-carboxamide (compd. 6);
5-(2-((5-(5-(difluoromethyl)-1,3,4-oxadiazol-2-yl)pyridin-2-yl)methyl)-2H-tetrazol-5-yl)benzo[d]oxazol-2-amine (compd. 7);
5-(1-(4-(5-(difluoromethyl)-1,3,4-oxadiazol-2-yl)benzyl)-1H-1,2,3-triazol-4-yl)-1H-benzo[d]imidazol-2-amine (compd. 8);
2-(6-((4-(2-chloro-1H-benzo[d]imidazol-6-yl)-1H-1,2,3-triazol-1-yl)methyl)pyridin-3-yl)-5-(difluoromethyl)-1,3,4-oxadiazole (compd. 9);
N-(4-(1-(4-(5-(difluoromethyl)-1,3,4-oxadiazol-2-yl)benzyl)-1H-1,2,3-triazol-4-yl)phenyl)-4,5-dihydro-1H-imidazol-2-amine (compd. 10);
5-(2-(4-(5-(difluoromethyl)-1,3,4-oxadiazol-2-yl)benzyl)-2H-tetrazol-5-yl)-1-methyl-1H-benzo[d]imidazol-2-amine (compd. 11);
5-(1-((5-(5-(difluoromethyl)-1,3,4-oxadiazol-2-yl)pyridin-2-yl)methyl)-1H-imidazol-4-yl)pyridin-2-amine (compd. 12);
5-(1-((5-(5-(difluoromethyl)-1,3,4-oxadiazol-2-yl)pyridin-2-yl)methyl)-1H-pyrazol-4-yl)pyridin-2-amine (compd. 13);
6-(1-(4-(5-(difluoromethyl)-1,3,4-oxadiazol-2-yl)benzyl)-1H-1,2,3-triazol-4-yl)benzo[d]thiazol-2-amine (compd. 14);
5-(1-(4-(5-(difluoromethyl)-1,3,4-oxadiazol-2-yl)-3-fluorobenzyl)-1H-1,2,3-triazol-4-yl)pyridin-2-amine (compd. 15);
N-(3-(1-(4-(5-(difluoromethyl)-1,3,4-oxadiazol-2-yl)-3-fluorobenzyl)-1H-1,2,3-triazol-4-yl)phenyl)morpholine-4-carboxamide (compd. 16);
5-(1-((5-(5-(difluoromethyl)-1,3,4-oxadiazol-2-yl)pyridin-2-yl)methyl)-1H-1,2,3-triazol-4-yl)-1-methyl-1H-benzo[d]imidazol-2-amine (compd. 17);
6-(1-((5-(5-(difluoromethyl)-1,3,4-oxadiazol-2-yl)pyridin-2-yl)methyl)-1H-1,2,3-triazol-4-yl)-N-ethyl-1H-benzo[d]imidazol-2-amine (compd. 18);
5-(1-((5-(5-(difluoromethyl)-1,3,4-oxadiazol-2-yl)pyridin-2-yl)methyl)-1H-1,2,3-triazol-4-yl)spiro[indoline-3,4′-piperidin]-2-one (compd. 19);
N-(4-(1-((5-(5-(difluoromethyl)-1,3,4-oxadiazol-2-yl)pyridin-2-yl)methyl)-1H-imidazol-4-yl)phenyl)-4,5-dihydro-1H-imidazol-2-amine (compd. 20);
5-(1-(4-(5-(difluoromethyl)-1,3,4-oxadiazol-2-yl)-2,3-difluorobenzyl)-1H-1,2,3-triazol-4-yl)pyridin-2-amine (compd. 21);
N-(4-(1-(4-(5-(difluoromethyl)-1,3,4-oxadiazol-2-yl)benzyl)-1H-imidazol-4-yl)phenyl)-4,5-dihydro-1H-imidazol-2-amine (compd. 22);
5-(1-(4-(5-(difluoromethyl)-1,3,4-oxadiazol-2-yl)benzyl)-1H-1,2,3-triazol-4-yl)-1-methyl-1H-benzo[d]imidazol-2-amine (compd. 23),
N-(4-(1-((5-(5-(difluoromethyl)-1,3,4-oxadiazol-2-yl)pyridin-2-yl)methyl)-1H-1,2,3-triazol-4-yl)phenyl)-4,5-dihydro-1H-imidazol-2-amine (compd. 24);
N-(5-(2-(4-(5-(difluoromethyl)-1,3,4-oxadiazol-2-yl)benzyl)-2H-tetrazol-5-yl)-2-hydroxyphenyl)morpholine-4-carboxamide (compd. 25);
5′-(1-((5-(5-(difluoromethyl)-1,3,4-oxadiazol-2-yl)pyridin-2-yl)methyl)-1H-1,2,3-triazol-4-yl)spiro[cyclopentane-1,3′-indolin]-2′-one (compd. 26);
7′-(2-((5-(5-(difluoromethyl)-1,3,4-oxadiazol-2-yl)pyridin-2-yl)methyl)-2H-tetrazol-5-yl)-1′,4′-dihydro-3′H-spiro[cyclopentane-1,2′-quinoxalin]-3′-one (compd. 27);
5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one (compd. 28);
3-(2-((5-(5-(difluoromethyl)-1,3,4-oxadiazol-2-yl)pyridin-2-yl)methyl)-2H-tetrazol-5-yl)benzamide (compd. 29);
6-(1-((5-(5-(difluoromethyl)-1,3,4-oxadiazol-2-yl)pyridin-2-yl)methyl)-1H-1,2,3-triazol-4-yl)-1H-benzo[d]imidazol-2-amine (compd. 30);
3-(5-(2-(4-(5-(difluoromethyl)-1,3,4-oxadiazol-2-yl)benzyl)-2H-tetrazol-5-yl)-2-hydroxyphenyl)-1,1-dimethylurea (compd. 31);
(R)-5-(1-(1-(5-(5-(difluoromethyl)-1,3,4-oxadiazol-2-yl)pyridin-2-yl)ethyl)-1H-1,2,3-triazol-4-yl)pyridin-2-amine (compd. 32);
(4-(2-(4-(5-(difluoromethyl)-1,3,4-oxadiazol-2-yl)-2,6-difluorobenzyl)-2H-tetrazol-5-yl)phenyl)methanamine (compd. 33);
6-(1-((5-(5-(difluoromethyl)-1,3,4-oxadiazol-2-yl)pyridin-2-yl)methyl)-1H-1,2,3-triazol-4-yl)-N-methylquinolin-2-amine (compd. 34);
2-amino-4-(2-(4-(5-(difluoromethyl)-1,3,4-oxadiazol-2-yl)benzyl)-2H-tetrazol-5-yl)phenol (compd. 35);
7′-(1-((5-(5-(difluoromethyl)-1,3,4-oxadiazol-2-yl)pyridin-2-yl)methyl)-1H-1,2,3-triazol-4-yl)-1′,4′-dihydro-3′H-spiro[piperidine-4,2′-quinoxalin]-3′-one (compd. 36);
N-(3-(1-((5-(5-(difluoromethyl)-1,3,4-oxadiazol-2-yl)pyridin-2-yl)methyl)-1H-1,2,3-triazol-4-yl)phenyl)acetamide (compd. 37);
5-(3-(2-((5-(5-(difluoromethyl)-1,3,4-oxadiazol-2-yl)pyridin-2-yl)methyl)-2H-tetrazol-5-yl)phenyl)thiazol-2-amine (compd. 38);
5-(1-(4-(5-(difluoromethyl)-1,3,4-oxadiazol-2-yl)-2,5-difluorobenzyl)-1H-1,2,3-triazol-4-yl)pyridin-2-amine (compd. 39);
6-(2-(4-(5-(difluoromethyl)-1,3,4-oxadiazol-2-yl)-2-fluorobenzyl)-2H-tetrazol-5-yl)isoindolin-1-one (compd. 40);
6′-(1-((5-(5-(difluoromethyl)-1,3,4-oxadiazol-2-yl)pyridin-2-yl)methyl)-1H-1,2,3-triazol-4-yl)-1′,4′-dihydro-3′H-spiro[piperidine-4,2′-quinoxalin]-3′-one (compd. 41);
5-(1-(4-(5-(difluoromethyl)-1,3,4-oxadiazol-2-yl)-2,6-difluorobenzyl)-1H-1,2,3-triazol-4-yl)pyridin-2-amine (compd. 42);
(4-(2-(4-(5-(difluoromethyl)-1,3,4-oxadiazol-2-yl)benzyl)-2H-tetrazol-5-yl)phenyl)methanamine (compd. 43);
(4-(1-(4-(5-(difluoromethyl)-1,3,4-oxadiazol-2-yl)-2,6-difluorobenzyl)-1H-1,2,3-triazol-4-yl)phenyl)methanamine (compd. 44);
5-(2-(4-(5-(difluoromethyl)-1,3,4-oxadiazol-2-yl)-2,6-difluorobenzyl)-2H-tetrazol-5-yl)pyridin-2-amine (compd. 45);
5-(1-(4-(5-(difluoromethyl)-1,3,4-oxadiazol-2-yl)benzyl)-1H-1,2,3-triazol-4-yl)spiro[indoline-3,4′-piperidin]-2-one (compd. 46);
N-(3-(1-(4-(5-(difluoromethyl)-1,3,4-oxadiazol-2-yl)-2-fluorobenzyl)-1H-1,2,3-triazol-4-yl)phenyl)morpholine-4-carboxamide (compd. 47);
5-(1-(4-(5-(difluoromethyl)-1,3,4-oxadiazol-2-yl)-2-fluorobenzyl)-1H-1,2,3-triazol-4-yl)pyridin-2-amine (compd. 48);
5-(1-(4-(5-(difluoromethyl)-1,3,4-oxadiazol-2-yl)benzyl)-1H-1,2,3-triazol-4-yl)spiro[indoline-3,3′-pyrrolidin]-2-one (compd. 49);
3-(2-(4-(5-(difluoromethyl)-1,3,4-oxadiazol-2-yl)-2-fluorobenzyl)-2H-tetrazol-5-yl)benzamide (compd. 50);
N-(3-(1-((5-(5-(difluoromethyl)-1,3,4-oxadiazol-2-yl)pyridin-2-yl)methyl)-1H-1,2,3-triazol-4-yl)phenyl)-4-methylpiperazine-1-carboxamide (compd. 51);
5-(1-(4-(5-(difluoromethyl)-1,3,4-oxadiazol-2-yl)benzyl)-1H-1,2,3-triazol-4-yl)pyridin-2-amine (compd. 52);
2-(difluoromethyl)-5-(6-((4-(2-methoxypyridin-3-yl)-1H-1,2,3-triazol-1-yl)methyl)pyridin-3-yl)-1,3,4-oxadiazole (compd. 53);
3-(2-(4-(5-(difluoromethyl)-1,3,4-oxadiazol-2-yl)-2,6-difluorobenzyl)-2H-tetrazol-5-yl)benzamide (compd. 54);
6-(2-(4-(5-(difluoromethyl)-1,3,4-oxadiazol-2-yl)benzyl)-2H-tetrazol-5-yl)isoindolin-1-one (compd. 55);
4-(2-(4-(5-(difluoromethyl)-1,3,4-oxadiazol-2-yl)benzyl)-2H-tetrazol-5-yl)phenol (compd. 56);
6-(2-(4-(5-(difluoromethyl)-1,3,4-oxadiazol-2-yl)-2,6-difluorobenzyl)-2H-tetrazol-5-yl)isoindolin-1-one (compd. 57);
2-(difluoromethyl)-5-(4-((5-(3-(4-methylpiperazin-1-yl)phenyl)-2H-tetrazol-2-yl)methyl)phenyl)-1,3,4-oxadiazole (compd. 58);
5-(1-(1-(5-(5-(difluoromethyl)-1,3,4-oxadiazol-2-yl)pyridin-2-yl)ethyl)-1H-1,2,3-triazol-4-yl)pyridin-2-amine (compd. 59);
6-(1-(4-(5-(difluoromethyl)-1,3,4-oxadiazol-2-yl)benzyl)-1H-1,2,3-triazol-4-yl)-N-ethyl-1H-benzo[d]imidazol-2-amine (compd. 60);
5′-(1-(4-(5-(difluoromethyl)-1,3,4-oxadiazol-2-yl)benzyl)-1H-1,2,3-triazol-4-yl)spiro[cyclopentane-1,3′-indolin]-2′-one (compd. 61);
N-(3-(4-(6-aminopyridin-3-yl)-1H-1,2,3-triazol-1-yl)-3-(4-(5-(difluoromethyl)-1,3,4-oxadiazol-2-yl)phenyl)propyl)methanesulfonamide (compd. 62);
N-(3-(1-(4-(5-(difluoromethyl)-1,3,4-oxadiazol-2-yl)-2,6-difluorobenzyl)-1H-1,2,3-triazol-4-yl)phenyl)-4-methylpiperazine-1-carboxamide (compd. 63);
5-(2-(4-(5-(difluoromethyl)-1,3,4-oxadiazol-2-yl)benzyl)-2H-tetrazol-5-yl)pyridin-2-amine (compd. 64);
5-(2-(4-(5-(difluoromethyl)-1,3,4-oxadiazol-2-yl)benzyl)-2H-tetrazol-5-yl)-2-methylpyridin-3-amine (compd. 65);
N-(3-(1-(1-(5-(5-(difluoromethyl)-1,3,4-oxadiazol-2-yl)pyridin-2-yl)ethyl)-1H-1,2,3-triazol-4-yl)phenyl)morpholine-4-carboxamide (compd. 66);
2-(3,5-difluoro-4-((4-(imidazo[1,2-b]pyridazin-3-yl)-1H-1,2,3-triazol-1-yl)methyl)phenyl)-5-(difluoromethyl)-1,3,4-oxadiazole (compd. 67);
N-(5-(2-((5-(5-(difluoromethyl)-1,3,4-oxadiazol-2-yl)pyrimidin-2-yl)methyl)-2H-tetrazol-5-yl)pyridin-2-yl)-2,2-difluoroacetamide (compd. 68);
(3-(2-((5-(5-(difluoromethyl)-1,3,4-oxadiazol-2-yl)pyridin-2-yl)methyl)-2H-tetrazol-5-yl)phenyl)(morpholino)methanone (compd. 69);
N-(3-(1-(4-(5-(difluoromethyl)-1,3,4-oxadiazol-2-yl)-2,6-difluorobenzyl)-1H-1,2,3-triazol-4-yl)phenyl)acetamide (compd. 70);
N-(3-(2-(4-(5-(difluoromethyl)-1,3,4-oxadiazol-2-yl)benzyl)-2H-tetrazol-5-yl)phenyl)morpholine-4-carboxamide (compd. 71);
2-amino-5-(2-(4-(5-(difluoromethyl)-1,3,4-oxadiazol-2-yl)benzyl)-2H-tetrazol-5-yl)benzamide (compd. 72);
5-(1-(4-(5-(difluoromethyl)-1,3,4-oxadiazol-2-yl)-2,6-difluorobenzyl)-1H-1,2,3-triazol-4-yl)pyridin-3-amine (compd. 73);
2-(difluoromethyl)-5-(6-((4-(imidazo[1,2-b]pyridazin-3-yl)-1H-1,2,3-triazol-1-yl)methyl)pyridin-3-yl)-1,3,4-oxadiazole (compd. 74);
3-(2-(4-(5-(difluoromethyl)-1,3,4-oxadiazol-2-yl)benzyl)-2H-tetrazol-5-yl)benzamide (compd. 75);
2-amino-5-(2-(4-(5-(difluoromethyl)-1,3,4-oxadiazol-2-yl)benzyl)-2H-tetrazol-5-yl)nicotinamide (compd. 76);
5-(2-((5-(5-(difluoromethyl)-1,3,4-oxadiazol-2-yl)pyrimidin-2-yl)methyl)-2H-tetrazol-5-yl)pyridin-2-amine (compd. 77);
N-(3-(1-(4-(5-(difluoromethyl)-1,3,4-oxadiazol-2-yl)-2,6-difluorobenzyl)-1H-1,2,3-triazol-4-yl)phenyl)morpholine-4-carboxamide (compd. 78);
5-(2-(4-(5-(difluoromethyl)-1,3,4-oxadiazol-2-yl)benzyl)-2H-tetrazol-5-yl)pyrimidin-2-amine (compd. 79);
3-(2-(4-(5-(difluoromethyl)-1,3,4-oxadiazol-2-yl)benzyl)-2H-tetrazol-5-yl)-N-(1-methylpiperidin-4-yl)benzamide (compd. 80);
3-(2-(4-(5-(difluoromethyl)-1,3,4-oxadiazol-2-yl)benzyl)-2H-tetrazol-5-yl)-N,N-dimethylbenzamide (compd. 81);
2-(4-((5-(5-bromopyridin-3-yl)-2H-tetrazol-2-yl)methyl)phenyl)-5-(difluoromethyl)-1,3,4-oxadiazole (compd. 82);
7-(2-(4-(5-(difluoromethyl)-1,3,4-oxadiazol-2-yl)benzyl)-2H-tetrazol-5-yl)-3,4-dihydroisoquinolin-1 (2H)-one (compd. 83);
7-(1-(4-(5-(difluoromethyl)-1,3,4-oxadiazol-2-yl)benzyl)-1H-1,2,3-triazol-4-yl)quinazolin-4-amine (compd. 84);
2-(difluoromethyl)-5-(6-((4-(thiophen-2-yl)-1H-1,2,3-triazol-1-yl)methyl)pyridin-3-yl)-1,3,4-oxadiazole (compd. 85);
N-(3-(1-(4-(5-(difluoromethyl)-1,3,4-oxadiazol-2-yl)-2,6-difluorobenzyl)-1H-1,2,3-triazol-4-yl)phenyl)-1-methylazetidine-3-carboxamide (compd. 86);
2-(difluoromethyl)-5-(4-((5-(4-(piperidin-1-ylmethyl)phenyl)-2H-tetrazol-2-yl)methyl)phenyl)-1,3,4-oxadiazole (compd. 87);
N-(5-(1-((5-(5-(difluoromethyl)-1,3,4-oxadiazol-2-yl)pyrimidin-2-yl)methyl)-1H-1,2,3-triazol-4-yl)pyridin-2-yl)-2,2-difluoroacetamide (compd. 88);
3-(2-(4-(5-(difluoromethyl)-1,3,4-oxadiazol-2-yl)-3-fluorobenzyl)-2H-tetrazol-5-yl)benzamide (compd. 89);
5-(2-(4-(5-(difluoromethyl)-1,3,4-oxadiazol-2-yl)benzyl)-2H-tetrazol-5-yl)pyridin-3-amine (compd. 90);
3-(2-(4-(5-(difluoromethyl)-1,3,4-oxadiazol-2-yl)benzyl)-2H-tetrazol-5-yl)-N-ethylbenzamide (compd. 91);
1-(3-(1-(4-(5-(difluoromethyl)-1,3,4-oxadiazol-2-yl)-2,6-difluorobenzyl)-1H-1,2,3-triazol-4-yl)phenyl)-3,3-dimethylazetidin-2-one (compd. 92);
(3-(2-(4-(5-(difluoromethyl)-1,3,4-oxadiazol-2-yl)-2-fluorobenzyl)-2H-tetrazol-5-yl)phenyl)(morpholino)methanone (compd. 93);
2-(4-(6-aminopyridin-3-yl)-1H-1,2,3-triazol-1-yl)-2-(4-(5-(difluoromethyl)-1,3,4-oxadiazol-2-yl)phenyl)ethan-1-ol (compd. 94);
N-(5-(2-(4-(5-(difluoromethyl)-1,3,4-oxadiazol-2-yl)-2,6-difluorobenzyl)-2H-tetrazol-5-yl)-2-hydroxyphenyl)morpholine-4-carboxamide (compd. 95);
3-(2-(4-(5-(difluoromethyl)-1,3,4-oxadiazol-2-yl)benzyl)-2H-tetrazol-5-yl)-N-(furan-2-ylmethyl)benzamide (compd. 96);
6-(2-(4-(5-(difluoromethyl)-1,3,4-oxadiazol-2-yl)-3-fluorobenzyl)-2H-tetrazol-5-yl)isoindolin-1-one (compd. 97);
N-(3-(2-(4-(5-(difluoromethyl)-1,3,4-oxadiazol-2-yl)-2,6-difluorobenzyl)-2H-tetrazol-5-yl)phenyl)morpholine-4-carboxamide (compd. 98);
5-(2-(4-(5-(difluoromethyl)-1,3,4-oxadiazol-2-yl)benzyl)-2H-tetrazol-5-yl)-N-ethylpyridin-2-amine (compd. 99);
(4-(3-(4-(5-(difluoromethyl)-1,3,4-oxadiazol-2-yl)-2,6-difluorobenzyl)-1,2,4-oxadiazol-5-yl)phenyl)methanamine (compd. 100);
(5-(1-(4-(5-(difluoromethyl)-1,3,4-oxadiazol-2-yl)-2,6-difluorobenzyl)-1H-1,2,3-triazol-4-yl)pyridin-2-yl)methanamine (compd. 101);
N-(5-(5-((5-(5-(difluoromethyl)-1,3,4-oxadiazol-2-yl)pyridin-2-yl)methyl)-1,3,4-thiadiazol-2-yl)pyridin-2-yl)-2,2-difluoroacetamide (compd. 102);
2-(difluoromethyl)-5-(4-((5-(4-(piperazin-1-yl)phenyl)-2H-tetrazol-2-yl)methyl)phenyl)-1,3,4-oxadiazole (compd. 103);
N-(3-(1-(1-(4-(5-(difluoromethyl)-1,3,4-oxadiazol-2-yl)phenyl)ethyl)-1H-1,2,3-triazol-4-yl)phenyl)morpholine-4-carboxamide (compd. 104);
2-(3,5-difluoro-4-((4-(2-methylpyridin-3-yl)-1H-1,2,3-triazol-1-yl)methyl)phenyl)-5-(difluoromethyl)-1,3,4-oxadiazole (compd. 105);
(R)-5-(1-(1-(4-(5-(difluoromethyl)-1,3,4-oxadiazol-2-yl)phenyl)ethyl)-1H-1,2,3-triazol-4-yl)pyridin-2-amine (compd. 106);
6-(2-((5-(5-(difluoromethyl)-1,3,4-oxadiazol-2-yl)pyrimidin-2-yl)methyl)-2H-tetrazol-5-yl)isoindolin-1-one (compd. 107);
2-(difluoromethyl)-5-(4-((5-(3-(4,5,6,7-tetrahydro-1H-imidazo[4,5-c]pyridin-2-yl)phenyl)-2H-tetrazol-2-yl)methyl)phenyl)-1,3,4-oxadiazole (compd. 108);
6-(1-(4-(5-(difluoromethyl)-1,3,4-oxadiazol-2-yl)benzyl)-1H-1,2,3-triazol-4-yl)isoindolin-1-one (compd. 109);
7′-(2-(4-(5-(difluoromethyl)-1,3,4-oxadiazol-2-yl)benzyl)-2H-tetrazol-5-yl)-1′,4′-dihydro-3′H-spiro[cyclopentane-1,2′-quinoxalin]-3′-one (compd. 110);
2-(difluoromethyl)-5-(4-((5-(4-(4,5,6,7-tetrahydropyrazolo[1,5-a]pyrazin-3-yl)phenyl)-2H-tetrazol-2-yl)methyl)phenyl)-1,3,4-oxadiazole (compd. 111);
(3-(2-(4-(5-(difluoromethyl)-1,3,4-oxadiazol-2-yl)-2,6-difluorobenzyl)-2H-tetrazol-5-yl)phenyl)(morpholino)methanone (compd. 112);
2-(difluoromethyl)-5-(4-((5-(quinolin-2-yl)-2H-tetrazol-2-yl)methyl)phenyl)-1,3,4-oxadiazole (compd. 113);
3-(1-(4-(5-(difluoromethyl)-1,3,4-oxadiazol-2-yl)-2,6-difluorobenzyl)-1H-1,2,3-triazol-4-yl)-N-ethylaniline (compd. 114);
2-(difluoromethyl)-5-(6-((4-(2-methylpyridin-3-yl)-1H-1,2,3-triazol-1-yl)methyl)pyridin-3-yl)-1,3,4-oxadiazole (compd. 115);
4-(2-(4-(5-(difluoromethyl)-1,3,4-oxadiazol-2-yl)benzyl)-2H-tetrazol-5-yl)benzamide (compd. 116);
5-(1-(1-(4-(5-(difluoromethyl)-1,3,4-oxadiazol-2-yl)phenyl)ethyl)-1H-1,2,3-triazol-4-yl)pyridin-2-amine (compd. 117);
2-(difluoromethyl)-5-(4-((5-(isoquinolin-4-yl)-2H-tetrazol-2-yl)methyl)phenyl)-1,3,4-oxadiazole (compd. 118);
N-(3-(1-((5-(5-(difluoromethyl)-1,3,4-oxadiazol-2-yl)pyridin-2-yl)methyl)-1H-pyrazol-4-yl)phenyl)morpholine-4-carboxamide (compd. 119);
(3-(2-(4-(5-(difluoromethyl)-1,3,4-oxadiazol-2-yl)benzyl)-2H-tetrazol-5-yl)phenyl)(morpholino)methanone (compd. 120);
4-(1-(4-(5-(difluoromethyl)-1,3,4-oxadiazol-2-yl)benzyl)-1H-1,2,3-triazol-4-yl)aniline (compd. 121);
2-(3,5-difluoro-4-((4-(thiophen-2-yl)-1H-1,2,3-triazol-1-yl)methyl)phenyl)-5-(difluoromethyl)-1,3,4-oxadiazole (compd. 122);
6′-(1-((5-(5-(difluoromethyl)-1,3,4-oxadiazol-2-yl)pyridin-2-yl)methyl)-1H-1,2,3-triazol-4-yl)spiro[cyclopentane-1,3′-indolin]-2′-one (compd. 123);
5-(1-(1-(4-(5-(difluoromethyl)-1,3,4-oxadiazol-2-yl)phenyl)-2-(pyrrolidin-1-yl)ethyl)-1H-1,2,3-triazol-4-yl)pyridin-2-amine (compd. 124);
5-(1-((5-(5-(difluoromethyl)-1,3,4-oxadiazol-2-yl)pyrimidin-2-yl)methyl)-1H-1,2,3-triazol-4-yl)pyridin-2-amine (compd. 125);
N-(5-(1-((5-(5-(difluoromethyl)-1,3,4-oxadiazol-2-yl)pyridin-2-yl)methyl)-1H-imidazol-4-yl)pyridin-2-yl)-2,2-difluoroacetamide (compd. 126);
2-(difluoromethyl)-5-(4-((5-(isoquinolin-7-yl)-2H-tetrazol-2-yl)methyl)phenyl)-1,3,4-oxadiazole (compd. 127);
2-(difluoromethyl)-5-(4-((5-(3,4-dimethoxyphenyl)-2H-tetrazol-2-yl)methyl)phenyl)-1,3,4-oxadiazole (compd. 128);
3-(2-(4-(5-(difluoromethyl)-1,3,4-oxadiazol-2-yl)benzyl)-2H-tetrazol-5-yl)aniline (compd. 129);
4-(5-(3-(2-((5-(5-(difluoromethyl)-1,3,4-oxadiazol-2-yl)pyridin-2-yl)methyl)-2H-tetrazol-5-yl)phenyl)thiazol-2-yl)morpholine (compd. 130);
2-(difluoromethyl)-5-(4-((4-(2-methoxypyridin-3-yl)-1H-1,2,3-triazol-1-yl)methyl)phenyl)-1,3,4-oxadiazole (compd. 131);
5-(1-(4-(5-(difluoromethyl)-1,3,4-oxadiazol-2-yl)benzyl)-1H-1,2,3-triazol-4-yl)benzo[d]thiazol-2-amine (compd. 132);
N-(5-(2-(4-(5-(difluoromethyl)-1,3,4-oxadiazol-2-yl)benzyl)-2H-tetrazol-5-yl)-2-methylpyridin-3-yl)acetamide (compd. 133);
5-(1-(2-chloro-4-(5-(difluoromethyl)-1,3,4-oxadiazol-2-yl)benzyl)-1H-1,2,3-triazol-4-yl)pyridin-2-amine (compd. 134);
5-(5-(4-(5-(difluoromethyl)-1,3,4-oxadiazol-2-yl)benzyl)-1,2,4-oxadiazol-3-yl)pyridin-2-amine (compd. 135);
2-(4-((4-(2-chloro-1H-benzo[d]imidazol-6-yl)-1H-1,2,3-triazol-1-yl)methyl)phenyl)-5-(difluoromethyl)-1,3,4-oxadiazole (compd. 136);
(3-(2-(4-(5-(difluoromethyl)-1,3,4-oxadiazol-2-yl)-3-fluorobenzyl)-2H-tetrazol-5-yl)phenyl)(morpholino)methanone (compd. 137);
5-((4-(1-(4-(5-(difluoromethyl)-1,3,4-oxadiazol-2-yl)benzyl)-1H-1,2,3-triazol-4-yl)benzyl)amino)-2-methoxynicotinamide (compd. 138);
N-(3-(1-(4-(5-(difluoromethyl)-1,3,4-oxadiazol-2-yl)benzyl)-1H-1,2,3-triazol-4-yl)phenyl)acetamide (compd. 139);
1-(3-(2-(4-(5-(difluoromethyl)-1,3,4-oxadiazol-2-yl)benzyl)-2H-tetrazol-5-yl)phenyl)ethan-1-one (compd. 140);
5-(3-(4-(5-(difluoromethyl)-1,3,4-oxadiazol-2-yl)-2,6-difluorobenzyl)-1,2,4-oxadiazol-5-yl)pyridin-2-amine (compd. 141);
6-(1-(4-(5-(difluoromethyl)-1,3,4-oxadiazol-2-yl)benzyl)-1H-1,2,3-triazol-4-yl)-N-methylquinolin-2-amine (compd. 142);
(R)-5-(1-(1-(4-(5-(difluoromethyl)-1,3,4-oxadiazol-2-yl)phenyl)butyl)-1H-1,2,3-triazol-4-yl)pyridin-2-amine (compd. 143);
2-amino-N-(3-(1-(4-(5-(difluoromethyl)-1,3,4-oxadiazol-2-yl)-2,6-difluorobenzyl)-1H-1,2,3-triazol-4-yl)phenyl)acetamide (compd. 144);
N-(3-(3-(4-(5-(difluoromethyl)-1,3,4-oxadiazol-2-yl)-2,6-difluorobenzyl)-1,2,4-oxadiazol-5-yl)phenyl)morpholine-4-carboxamide (compd. 145);
N-(3-(1-(4-(5-(difluoromethyl)-1,3,4-oxadiazol-2-yl)benzyl)-1H-1,2,3-triazol-4-yl)phenyl)-4-methylpiperazine-1-carboxamide (compd. 146);
2-(difluoromethyl)-5-(4-((5-(1-(pyridin-2-yl)cyclopropyl)-2H-tetrazol-2-yl)methyl)phenyl)-1,3,4-oxadiazole (compd. 147);
2-(difluoromethyl)-5-(4-((5-(6-(piperazin-1-yl)pyridin-3-yl)-2H-tetrazol-2-yl)methyl)phenyl)-1,3,4-oxadiazole (compd. 148);
N-(3-(1-(4-(5-(difluoromethyl)-1,3,4-oxadiazol-2-yl)benzyl)-1H-1,2,3-triazol-4-yl)phenyl)-1-methylazetidine-3-carboxamide (compd. 149);
2-(difluoromethyl)-5-(4-((5-(2-nitrophenyl)-2H-tetrazol-2-yl)methyl)phenyl)-1,3,4-oxadiazole (compd. 150);
5-(1-(4-(5-(difluoromethyl)-1,3,4-oxadiazol-2-yl)benzyl)-1H-imidazol-4-yl)pyridin-2-amine (compd. 151);
5-(2-(4-(5-(difluoromethyl)-1,3,4-oxadiazol-2-yl)benzyl)-2H-tetrazol-5-yl)benzo[d]oxazol-2-amine (compd. 152);
2-(difluoromethyl)-5-(4-((5-(isoquinolin-5-yl)-2H-tetrazol-2-yl)methyl)phenyl)-1,3,4-oxadiazole (compd. 153);
5-((4-(1-(4-(5-(difluoromethyl)-1,3,4-oxadiazol-2-yl)-2,6-difluorobenzyl)-1H-1,2,3-triazol-4-yl)benzyl)amino)-2-methoxynicotinamide (compd. 154);
(5-(1-(4-(5-(difluoromethyl)-1,3,4-oxadiazol-2-yl)benzyl)-1H-1,2,3-triazol-4-yl)pyridin-2-yl)methanamine (compd. 155);
N-(3-(2-(4-(5-(difluoromethyl)-1,3,4-oxadiazol-2-yl)benzyl)-2H-tetrazol-5-yl)phenyl)benzamide (compd. 156);
7′-(2-(4-(5-(difluoromethyl)-1,3,4-oxadiazol-2-yl)benzyl)-2H-tetrazol-5-yl)-1′,4′-dihydro-3′H-spiro[cyclohexane-1,2′-quinoxalin]-3′-one (compd. 157);
5-(1-(1-(4-(5-(difluoromethyl)-1,3,4-oxadiazol-2-yl)phenyl)-3,3,3-trifluoropropyl)-1H-1,2,3-triazol-4-yl)pyridin-2-amine (compd. 158);
(R)-2-(difluoromethyl)-5-(4-((5-(6-(3-methylpiperazin-1-yl)pyridin-3-yl)-2H-tetrazol-2-yl)methyl)phenyl)-1,3,4-oxadiazole (compd. 159);
2-amino-4-(2-(4-(5-(difluoromethyl)-1,3,4-oxadiazol-2-yl)-2,6-difluorobenzyl)-2H-tetrazol-5-yl)phenyl morpholine-4-carboxylate (compd. 160);
6-(1-(4-(5-(difluoromethyl)-1,3,4-oxadiazol-2-yl)benzyl)-1H-1,2,3-triazol-4-yl)spiro[indoline-3,4′-piperidin]-2-one (compd. 161);
5-(1-(4-(5-(difluoromethyl)-1,3,4-oxadiazol-2-yl)benzyl)-1H-1,2,3-triazol-4-yl)-1,3-dimethyl-1,3-dihydro-2H-benzo[d]imidazol-2-imine (compd. 162);
3-(2-(4-(5-(difluoromethyl)-1,3,4-oxadiazol-2-yl)benzyl)-2H-tetrazol-5-yl)-4-fluoro-N,N-dimethylbenzenesulfonamide (compd. 163);
4-(2-(4-(5-(difluoromethyl)-1,3,4-oxadiazol-2-yl)benzyl)-2H-tetrazol-5-yl)-N1-methylbenzene-1,2-diamine (compd. 164);
N-(3-(1-(1-(4-(5-(difluoromethyl)-1,3,4-oxadiazol-2-yl)-2-fluorophenyl)ethyl)-1H-1,2,3-triazol-4-yl)phenyl)morpholine-4-carboxamide (compd. 165);
6-(1-(4-(5-(difluoromethyl)-1,3,4-oxadiazol-2-yl)benzyl)-1H-1,2,3-triazol-4-yl)-1-methyl-1H-benzo[d]imidazol-2-amine (compd. 166);
5-(2-(4-(5-(difluoromethyl)-1,3,4-oxadiazol-2-yl)benzyl)-2H-tetrazol-5-yl)isoindolin-1-one (compd. 167);
5-(2-(4-(5-(difluoromethyl)-1,3,4-oxadiazol-2-yl)benzyl)-2H-tetrazol-5-yl)-1,3-dihydro-2H-benzo[d]imidazol-2-one (compd. 168);
2-(difluoromethyl)-5-(4-((4-(4-((4-(ethylsulfonyl)piperazin-1-yl)methyl)phenyl)-1H-1,2,3-triazol-1-yl)methyl)-3,5-difluorophenyl)-1,3,4-oxadiazole (compd. 169);
1-(5-(5-(4-(5-(difluoromethyl)-1,3,4-oxadiazol-2-yl)benzyl)-1,3,4-oxadiazol-2-yl)pyridin-2-yl)-3-methylurea (compd. 170);
(S)-5-(1-(1-(5-(5-(difluoromethyl)-1,3,4-oxadiazol-2-yl)pyridin-2-yl)ethyl)-1H-1,2,3-triazol-4-yl)pyridin-2-amine (compd. 171);
tert-butyl (2-((3-(1-(4-(5-(difluoromethyl)-1,3,4-oxadiazol-2-yl)-2,6-difluorobenzyl)-1H-1,2,3-triazol-4-yl)phenyl)amino)-2-oxoethyl)carbamate (compd. 172);
7-(2-(4-(5-(difluoromethyl)-1,3,4-oxadiazol-2-yl)benzyl)-2H-tetrazol-5-yl)-2-methyl-3,4-dihydroisoquinolin-1 (2H)-one (compd. 173);
4-(6-(1-(4-(5-(difluoromethyl)-1,3,4-oxadiazol-2-yl)benzyl)-1H-1,2,3-triazol-4-yl)-1H-benzo[d]imidazol-2-yl)morpholine (compd. 174);
1-(3-(1-(4-(5-(difluoromethyl)-1,3,4-oxadiazol-2-yl)-2,6-difluorobenzyl)-1H-1,2,3-triazol-4-yl)phenyl)thiourea (compd. 175);
N-(5-(2-(4-(5-(difluoromethyl)-1,3,4-oxadiazol-2-yl)benzyl)-2H-tetrazol-5-yl)-2-(methylamino)phenyl)morpholine-4-carboxamide (compd. 176);
tert-butyl 5-(1-(4-(5-(difluoromethyl)-1,3,4-oxadiazol-2-yl)benzyl)-1H-1,2,3-triazol-4-yl)-2-oxospiro[indoline-3,3′-pyrrolidine]-1′-carboxylate (compd. 177);
6-(1-(4-(5-(difluoromethyl)-1,3,4-oxadiazol-2-yl)benzyl)-1H-1,2,3-triazol-4-yl)thieno[2,3-d]pyrimidin-4-amine (compd. 178);
N-(4-(1-(4-(5-(difluoromethyl)-1,3,4-oxadiazol-2-yl)-2,6-difluorobenzyl)-1H-1,2,3-triazol-4-yl)benzyl)-N-methyl-1-(pyridin-4-yl)methanamine (compd. 179);
3-(1-(4-(5-(difluoromethyl)-1,3,4-oxadiazol-2-yl)benzyl)-1H-1,2,3-triazol-4-yl)-N-ethylaniline (compd. 180);
2-(difluoromethyl)-5-(4-((5-(2-fluorophenyl)-2H-tetrazol-2-yl)methyl)phenyl)-1,3,4-oxadiazole (compd. 181);
(S)-2-(difluoromethyl)-5-(4-((5-(6-(3-methylpiperazin-1-yl)pyridin-3-yl)-2H-tetrazol-2-yl)methyl)phenyl)-1,3,4-oxadiazole (compd. 182);
N-(3-(1-(4-(5-(difluoromethyl)-1,3,4-oxadiazol-2-yl)-2,6-difluorobenzyl)-1H-1,2,3-triazol-4-yl)phenyl)-N-(furan-2-ylmethyl)acetamide (compd. 183);
N-(3-(1-(1-(4-(5-(difluoromethyl)-1,3,4-oxadiazol-2-yl)phenyl)propyl)-1H-1,2,3-triazol-4-yl)phenyl)morpholine-4-carboxamide (compd. 184);
5-(1-(1-(4-(5-(difluoromethyl)-1,3,4-oxadiazol-2-yl)-2-fluorophenyl)ethyl)-1H-1,2,3-triazol-4-yl)pyridin-2-amine (compd. 185);
5-(1-(1-(4-(5-(difluoromethyl)-1,3,4-oxadiazol-2-yl)-3-fluorophenyl)ethyl)-1H-1,2,3-triazol-4-yl)pyridin-2-amine (compd. 186);
2-(difluoromethyl)-5-(2-((5-(thiophen-2-yl)-2H-tetrazol-2-yl)methyl)pyrimidin-5-yl)-1,3,4-oxadiazole (compd. 187);
2-(4-((5-(3-(1H-pyrazol-1-yl)phenyl)-2H-tetrazol-2-yl)methyl)phenyl)-5-(difluoromethyl)-1,3,4-oxadiazole (compd. 188);
N-(3-(1-(1-(4-(5-(difluoromethyl)-1,3,4-oxadiazol-2-yl)-3-fluorophenyl)ethyl)-1H-1,2,3-triazol-4-yl)phenyl)morpholine-4-carboxamide (compd. 189);
2-(difluoromethyl)-5-(4-((4-(2-(pyrrolidin-1-yl)-1H-benzo[d]imidazol-6-yl)-1H-1,2,3-triazol-1-yl)methyl)phenyl)-1,3,4-oxadiazole (compd. 190);
(4-(3-(4-(5-(difluoromethyl)-1,3,4-oxadiazol-2-yl)benzyl)-1,2,4-oxadiazol-5-yl)phenyl)methanamine (compd. 191);
3-(1-(4-(5-(difluoromethyl)-1,3,4-oxadiazol-2-yl)benzyl)-1H-1,2,3-triazol-4-yl)aniline (compd. 192);
5-(1-(1-(4-(5-(difluoromethyl)-1,3,4-oxadiazol-2-yl)phenyl)butyl)-1H-1,2,3-triazol-4-yl)pyridin-2-amine (compd. 193);
5-(1-(1-(4-(5-(difluoromethyl)-1,3,4-oxadiazol-2-yl)phenyl)propyl)-1H-1,2,3-triazol-4-yl)pyridin-2-amine (compd. 194);
6′-(2-(4-(5-(difluoromethyl)-1,3,4-oxadiazol-2-yl)benzyl)-2H-tetrazol-5-yl)-1′,4′-dihydro-3′H-spiro[cyclopentane-1,2′-quinoxalin]-3′-one (compd. 195);
4-(2-(4-(5-(difluoromethyl)-1,3,4-oxadiazol-2-yl)-2,6-difluorobenzyl)-2H-tetrazol-5-yl)-2-(morpholine-4-carboxamido)phenyl morpholine-4-carboxylate (compd. 196);
3-(1-(4-(5-(difluoromethyl)-1,3,4-oxadiazol-2-yl)benzyl)-1H-imidazol-4-yl)aniline (compd. 197);
5-(1-((6-(5-(difluoromethyl)-1,3,4-oxadiazol-2-yl)pyridazin-3-yl)methyl)-1H-1,2,3-triazol-4-yl)pyridin-2-amine (compd. 198);
N-(5-(2-(4-(5-(difluoromethyl)-1,3,4-oxadiazol-2-yl)benzyl)-2H-tetrazol-5-yl)pyridin-3-yl)morpholine-4-carboxamide (compd. 199);
5-(3-(2-(4-(5-(difluoromethyl)-1,3,4-oxadiazol-2-yl)benzyl)-2H-tetrazol-5-yl)phenyl)thiazol-2-amine (compd. 200);
N-(4-(2-(4-(5-(difluoromethyl)-1,3,4-oxadiazol-2-yl)-2,6-difluorobenzyl)-2H-tetrazol-5-yl)benzyl)-N-methyl-1-(pyridin-4-yl)methanamine (compd. 201);
5-(5-(4-(5-(difluoromethyl)-1,3,4-oxadiazol-2-yl)benzyl)isoxazol-3-yl)pyridin-2-amine (compd. 202);
6-(2-(4-(5-(difluoromethyl)-1,3,4-oxadiazol-2-yl)benzyl)-2H-tetrazol-5-yl)-2,3-dihydro-1H-inden-1-one (compd. 203);
2-(difluoromethyl)-5-(4-((5-(4-methoxyphenyl)-2H-tetrazol-2-yl)methyl)phenyl)-1,3,4-oxadiazole (compd. 204);
N-(3-(1-(1-(4-(5-(difluoromethyl)-1,3,4-oxadiazol-2-yl)phenyl)butyl)-1H-1,2,3-triazol-4-yl)phenyl)morpholine-4-carboxamide (compd. 205);
N-(4-(3-(4-(5-(difluoromethyl)-1,3,4-oxadiazol-2-yl)-2,6-difluorobenzyl)-1,2,4-oxadiazol-5-yl)benzyl)-2,2-difluoro-N-methylacetamide (compd. 206);
2-(4-((5-(benzo[b]thiophen-3-yl)-2H-tetrazol-2-yl)methyl)phenyl)-5-(difluoromethyl)-1,3,4-oxadiazole (compd. 207);
4-(2-(4-(5-(difluoromethyl)-1,3,4-oxadiazol-2-yl)benzyl)-2H-tetrazol-5-yl)-2,3-dihydro-1H-inden-1-one (compd. 208);
6′-(2-(4-(5-(difluoromethyl)-1,3,4-oxadiazol-2-yl)benzyl)-2H-tetrazol-5-yl)-1′,4′-dihydro-3′H-spiro[cyclohexane-1,2′-quinoxalin]-3′-one (compd. 209);
5-(2-(4-(5-(difluoromethyl)-1,3,4-oxadiazol-2-yl)benzyl)-2H-tetrazol-5-yl)-1-methyl-1,3-dihydro-2H-benzo[d]imidazol-2-one (compd. 210);
5-(1-(4-(5-(difluoromethyl)-1,3,4-oxadiazol-2-yl)benzyl)-1H-pyrazol-4-yl)pyridin-2-amine (compd. 211);
2-(difluoromethyl)-5-(4-((5-(6-(4-methylpiperazin-1-yl)pyridin-3-yl)-2H-tetrazol-2-yl)methyl)phenyl)-1,3,4-oxadiazole (compd. 212);
2-(difluoromethyl)-5-(4-((5-(4-(4-methylpiperazin-1-yl)phenyl)-2H-tetrazol-2-yl)methyl)phenyl)-1,3,4-oxadiazole (compd. 213);
2-(3,5-difluoro-4-((4-(4-((3-(trifluoromethyl)azetidin-1-yl)methyl)phenyl)-1H-1,2,3-triazol-1-yl)methyl)phenyl)-5-(difluoromethyl)-1,3,4-oxadiazole (compd. 214);
N-(4-(2-(4-(5-(difluoromethyl)-1,3,4-oxadiazol-2-yl)benzyl)-2H-tetrazol-5-yl)benzyl)-N-methyl-1-(pyridin-4-yl)methanamine (compd. 215);
tert-butyl 5-(1-(4-(5-(difluoromethyl)-1,3,4-oxadiazol-2-yl)benzyl)-1H-1,2,3-triazol-4-yl)-2-oxospiro[indoline-3,4′-piperidine]-1′-carboxylate (compd. 216);
2-(4-(1-(4-(5-(difluoromethyl)-1,3,4-oxadiazol-2-yl)benzyl)-1H-1,2,3-triazol-4-yl)phenyl)-1,1,3,3-tetramethylguanidine (compd. 217);
5-(5-(4-(5-(difluoromethyl)-1,3,4-oxadiazol-2-yl)benzyl)-1,3,4-oxadiazol-2-yl)pyridin-2-amine (compd. 218);
2-(difluoromethyl)-5-(4-((5-(2-(pyridin-4-yl)propan-2-yl)-2H-tetrazol-2-yl)methyl)phenyl)-1,3,4-oxadiazole (compd. 219);
2-(difluoromethyl)-5-(4-((5-(furan-2-yl)-2H-tetrazol-2-yl)methyl)phenyl)-1,3,4-oxadiazole (compd. 220);
5-(1-(1-(4-(5-(difluoromethyl)-1,3,4-oxadiazol-2-yl)phenyl)-2-phenylethyl)-1H-1,2,3-triazol-4-yl)pyridin-2-amine (compd. 221);
2-(4-((4-(1H-indazol-6-yl)-1H-1,2,3-triazol-1-yl)methyl)phenyl)-5-(difluoromethyl)-1,3,4-oxadiazole (compd. 222);
3-(1-(4-(5-(difluoromethyl)-1,3,4-oxadiazol-2-yl)benzyl)-1H-imidazol-4-yl)benzamide (compd. 223);
2-(difluoromethyl)-5-(4-((5-(3-fluoro-4-(piperazin-1-yl)phenyl)-2H-tetrazol-2-yl)methyl)phenyl)-1,3,4-oxadiazole (compd. 224);
5-(2-(4-(5-(difluoromethyl)-1,3,4-oxadiazol-2-yl)benzyl)-2H-tetrazol-5-yl)benzo[d]oxazol-2 (3H)-one (compd. 225);
3-(5-(4-(5-(difluoromethyl)-1,3,4-oxadiazol-2-yl)benzyl)-1,2,4-oxadiazol-3-yl)benzamide (compd. 226);
N-(3-(1-(4-(5-(difluoromethyl)-1,3,4-oxadiazol-2-yl)benzyl)-1H-pyrazol-4-yl)phenyl)morpholine-4-carboxamide (compd. 227);
N-(3-(3-(4-(5-(difluoromethyl)-1,3,4-oxadiazol-2-yl)benzyl)-1,2,4-oxadiazol-5-yl)phenyl)morpholine-4-carboxamide (compd. 228);
7-(2-(1-(4-(5-(difluoromethyl)-1,3,4-oxadiazol-2-yl)phenyl)ethyl)-2H-tetrazol-5-yl)-2-methyl-3,4-dihydroisoquinolin-1 (2H)-one (compd. 229);
(4-(2-(4-(5-(difluoromethyl)-1,3,4-oxadiazol-2-yl)benzyl)-2H-tetrazol-5-yl)phenyl)(morpholino)methanone (compd. 230);
5-(1-(2-(4-chlorophenyl)-1-(4-(5-(difluoromethyl)-1,3,4-oxadiazol-2-yl)phenyl)ethyl)-1H-1,2,3-triazol-4-yl)pyridin-2-amine (compd. 231);
4-(2-(4-(5-(difluoromethyl)-1,3,4-oxadiazol-2-yl)benzyl)-2H-tetrazol-5-yl)-N-(1-methylpiperidin-4-yl)benzamide (compd. 232);
2-(difluoromethyl)-5-(4-((4-(2-methoxyphenyl)-1H-1,2,3-triazol-1-yl)methyl)phenyl)-1,3,4-oxadiazole (compd. 233);
2-(difluoromethyl)-5-(4-((4-phenyl-1H-1,2,3-triazol-1-yl)methyl)phenyl)-1,3,4-oxadiazole (compd. 234);
5-(1-(1-(4-(5-(difluoromethyl)-1,3,4-oxadiazol-2-yl)phenyl)pentyl)-1H-1,2,3-triazol-4-yl)pyridin-2-amine (compd. 235);
5-(1-(1-(4-(5-(difluoromethyl)-1,3,4-oxadiazol-2-yl)phenyl)-2-phenoxyethyl)-1H-1,2,3-triazol-4-yl)pyridin-2-amine (compd. 236);
8-(1-(4-(5-(difluoromethyl)-1,3,4-oxadiazol-2-yl)-2,6-difluorobenzyl)-1H-1,2,3-triazol-4-yl)-4-methyl-1,3,4,5-tetrahydro-2H-benzo[e][1,4]diazepin-2-one (compd. 237);
2-(difluoromethyl)-5-(4-((5-phenyl-1,3,4-thiadiazol-2-yl)methyl)phenyl)-1,3,4-oxadiazole (compd. 238);
N-(cyclopropylmethyl)-1-(4-(1-(4-(5-(difluoromethyl)-1,3,4-oxadiazol-2-yl)-2,6-difluorobenzyl)-1H-1,2,3-triazol-4-yl)benzoyl)piperidine-3-carboxamide (compd. 239);
tert-butyl 3-(3-(2-(4-(5-(difluoromethyl)-1,3,4-oxadiazol-2-yl)benzyl)-2H-tetrazol-5-yl)phenyl)-6,7-dihydropyrazolo[1,5-a]pyrazine-5 (4H)-carboxylate (compd. 240);
2-(difluoromethyl)-5-(4-((4-(6-fluoro-2-methylpyridin-3-yl)-1H-1,2,3-triazol-1-yl)methyl)phenyl)-1,3,4-oxadiazole (compd. 241);
5-(1-(2-cyclobutyl-1-(4-(5-(difluoromethyl)-1,3,4-oxadiazol-2-yl)phenyl)ethyl)-1H-1,2,3-triazol-4-yl)pyridin-2-amine (compd. 242);
5-(5-((4-(5-(difluoromethyl)-1,3,4-oxadiazol-2-yl)phenyl)difluoromethyl)-1,2,4-oxadiazol-3-yl)pyridin-2-amine (compd. 243);
N-(3-(1-(1-(4-(5-(difluoromethyl)-1,3,4-oxadiazol-2-yl)phenyl)pentyl)-1H-1,2,3-triazol-4-yl)phenyl)morpholine-4-carboxamide (compd. 244);
6-(1-(4-(5-(difluoromethyl)-1,3,4-oxadiazol-2-yl)benzyl)-1H-1,2,3-triazol-4-yl)-3,3-dimethylisoindolin-1-one (compd. 245);
2-(4-((5-([1,1′-biphenyl]-3-yl)-2H-tetrazol-2-yl)methyl)phenyl)-5-(difluoromethyl)-1,3,4-oxadiazole (compd. 246);
5-(3-(4-(5-(difluoromethyl)-1,3,4-oxadiazol-2-yl)benzyl)-1,2,4-oxadiazol-5-yl)pyridin-2-amine (compd. 247);
2-(difluoromethyl)-5-(4-((4-(3-fluorophenyl)-1H-1,2,3-triazol-1-yl)methyl)phenyl)-1,3,4-oxadiazole (compd. 248);
5-(2-(4-(5-(difluoromethyl)-1,3,4-oxadiazol-2-yl)benzyl)-2H-tetrazol-5-yl)-N,N-dimethylbenzo[d]oxazol-2-amine (compd. 249);
(S)-5-(1-(1-(4-(5-(difluoromethyl)-1,3,4-oxadiazol-2-yl)phenyl)butyl)-1H-1,2,3-triazol-4-yl)pyridin-2-amine (compd. 250);
2-(difluoromethyl)-5-(4-((5-(pyridin-2-ylmethyl)-2H-tetrazol-2-yl)methyl)phenyl)-1,3,4-oxadiazole (compd. 251);
5-(2-((5-(5-(difluoromethyl)-1,3,4-oxadiazol-2-yl)pyrimidin-2-yl)methyl)-2H-tetrazol-5-yl)-1-methyl-1H-benzo[d]imidazol-2-amine (compd. 252)
4-(5-(3-(2-(4-(5-(difluoromethyl)-1,3,4-oxadiazol-2-yl)benzyl)-2H-tetrazol-5-yl)phenyl)thiazol-2-yl)morpholine (compd. 253);
N-(4-(3-(4-(5-(difluoromethyl)-1,3,4-oxadiazol-2-yl)-2,6-difluorobenzyl)-1,2,4-oxadiazol-5-yl)benzyl)-N-methyl-1-(pyridin-4-yl)methanamine (compd. 254);
(S)-5-(1-(1-(4-(5-(difluoromethyl)-1,3,4-oxadiazol-2-yl)phenyl)ethyl)-1H-1,2,3-triazol-4-yl)pyridin-2-amine (compd. 255);
2-(difluoromethyl)-5-(4-((5-(1-phenylcyclopropyl)-2H-tetrazol-2-yl)methyl)phenyl)-1,3,4-oxadiazole (compd. 256);
1-(4-(2-(4-(5-(difluoromethyl)-1,3,4-oxadiazol-2-yl)benzyl)-2H-tetrazol-5-yl)piperidin-1-yl)ethan-1-one (compd. 257);
N-(5-(2-(4-(5-(difluoromethyl)-1,3,4-oxadiazol-2-yl)benzyl)-2H-tetrazol-5-yl)-2-(phenylthio)phenyl)morpholine-4-carboxamide (compd. 258);
N-(4-(3-(4-(5-(difluoromethyl)-1,3,4-oxadiazol-2-yl)benzyl)-1,2,4-oxadiazol-5-yl)benzyl)-2,2-difluoro-N-methylacetamide (compd. 259);
3-(2-(4-(5-(difluoromethyl)-1,3,4-oxadiazol-2-yl)benzyl)-2H-tetrazol-5-yl)benzoic acid (compd. 260);
2-(difluoromethyl)-5-(4-((5-(thiophen-2-yl)-2H-tetrazol-2-yl)methyl)phenyl)-1,3,4-oxadiazole (compd. 261);
3-(3-(4-(5-(difluoromethyl)-1,3,4-oxadiazol-2-yl)benzyl)-1,2,4-oxadiazol-5-yl)benzamide (compd. 262);
2-(4-((5-(2,4-dichlorophenyl)-2H-tetrazol-2-yl)methyl)phenyl)-5-(difluoromethyl)-1,3,4-oxadiazole (compd. 263);
N-(3-(1-((5-(5-(difluoromethyl)-1,3,4-oxadiazol-2-yl)pyridin-2-yl)methyl)-1H-imidazol-4-yl)phenyl)morpholine-4-carboxamide (compd. 264);
tert-butyl 5-(1-((5-(5-(difluoromethyl)-1,3,4-oxadiazol-2-yl)pyridin-2-yl)methyl)-1H-1,2,3-triazol-4-yl)-2-oxospiro[indoline-3,3′-pyrrolidine]-1′-carboxylate enantiomer A (compd. 265);
tert-butyl 5-(1-((5-(5-(difluoromethyl)-1,3,4-oxadiazol-2-yl)pyridin-2-yl)methyl)-1H-1,2,3-triazol-4-yl)-2-oxospiro[indoline-3,3′-pyrrolidine]-1′-carboxylate enantiomer B (compd. 266);
N-(3-(1-(4-(5-(difluoromethyl)-1,3,4-oxadiazol-2-yl)benzyl)-1H-imidazol-4-yl)phenyl)morpholine-4-carboxamide (compd. 267);
tert-butyl 7′-(1-(4-(5-(difluoromethyl)-1,3,4-oxadiazol-2-yl)benzyl)-1H-1,2,3-triazol-4-yl)-3′-oxo-3′,4′-dihydro-1′H-spiro[piperidine-4,2′-quinoxaline]-1-carboxylate (compd. 268);
N-(4-(1-(4-(5-(difluoromethyl)-1,3,4-oxadiazol-2-yl)benzyl)-1H-pyrazol-4-yl)phenyl)-4,5-dihydro-1H-imidazol-2-amine (compd. 269);
N-(4-(1-((5-(5-(difluoromethyl)-1,3,4-oxadiazol-2-yl)pyridin-2-yl)methyl)-1H-pyrazol-4-yl)phenyl)-4,5-dihydro-1H-imidazol-2-amine (compd. 270);
7′-(1-(4-(5-(difluoromethyl)-1,3,4-oxadiazol-2-yl)benzyl)-1H-1,2,3-triazol-4-yl)-1′,4′-dihydro-3′H-spiro[piperidine-4,2′-quinoxalin]-3′-one (compd. 271);
tert-butyl 2-(3-(2-(4-(5-(difluoromethyl)-1,3,4-oxadiazol-2-yl)benzyl)-2H-tetrazol-5-yl)phenyl)-1,4,6,7-tetrahydro-5H-imidazo[4,5-c]pyridine-5-carboxylate (compd. 272);
5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one enantiomer A (compd. 273);
5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one enantiomer B (compd. 274);
3-(1-(4-(5-(difluoromethyl)-1,3,4-oxadiazol-2-yl)benzyl)-1H-1,2,3-triazol-4-yl)benzoic acid (compd. 275);
2-(difluoromethyl)-5-(6-((5-(3-(4,5,6,7-tetrahydro-1H-imidazo[4,5-c]pyridin-2-yl)phenyl)-2H-tetrazol-2-yl)methyl)pyridin-3-yl)-1,3,4-oxadiazole (compd. 276);
6′-(1-(4-(5-(difluoromethyl)-1,3,4-oxadiazol-2-yl)benzyl)-1H-1,2,3-triazol-4-yl)-1′,4′-dihydro-3′H-spiro[piperidine-4,2′-quinoxalin]-3′-one (compd. 277);
6-(2-(4-(5-(difluoromethyl)-1,3,4-oxadiazol-2-yl)benzyl)-2H-tetrazol-5-yl)quinazolin-2-amine (compd. 278);
tert-butyl 6′-(1-(4-(5-(difluoromethyl)-1,3,4-oxadiazol-2-yl)benzyl)-1H-1,2,3-triazol-4-yl)-3′-oxo-3′,4′-dihydro-1′H-spiro[piperidine-4,2′-quinoxaline]-1-carboxylate (compd. 279);
2-(difluoromethyl)-5-(4-((4-(imidazo[1,2-b]pyridazin-3-yl)-1H-1,2,3-triazol-1-yl)methyl)phenyl)-1,3,4-oxadiazole (compd. 280);
4-(1-(4-(5-(difluoromethyl)-1,3,4-oxadiazol-2-yl)benzyl)-1H-1,2,3-triazol-4-yl)-N,N-dimethylaniline (compd. 281);
N-(4-(1-(4-(5-(difluoromethyl)-1,3,4-oxadiazol-2-yl)benzyl)-1H-1,2,3-triazol-4-yl)benzyl)-N-methyl-1-(pyridin-4-yl)methanamine (compd. 282);
1-((1-(4-(5-(difluoromethyl)-1,3,4-oxadiazol-2-yl)-2,6-difluorobenzyl)-1H-1,2,3-triazol-4-yl)methyl)-1-ethyl-3-(2-methoxypyridin-3-yl)urea (compd. 283);
5-(5-((4-(5-(difluoromethyl)-1,3,4-oxadiazol-2-yl)-2,6-difluorophenyl)thio)-4-methyl-4H-1,2,4-triazol-3-yl)pyridin-2-amine (compd. 284);
5-(5-((4-(5-(difluoromethyl)-1,3,4-oxadiazol-2-yl)phenyl)thio)-4-methyl-4H-1,2,4-triazol-3-yl)pyridin-2-amine (compd. 285);
5-((4-(4-(4-(5-(difluoromethyl)-1,3,4-oxadiazol-2-yl)benzyl)-1H-1,2,3-triazol-1-yl)benzyl)amino)-2-methoxynicotinamide (compd. 286);
2-(difluoromethyl)-5-(4-((5-(pyrimidin-2-yl)-1H-tetrazol-1-yl)methyl)phenyl)-1,3,4-oxadiazole (compd. 287);
2-(4-((5-(benzo[b]thiophen-3-yl)-1H-tetrazol-1-yl)methyl)phenyl)-5-(difluoromethyl)-1,3,4-oxadiazole (compd. 288);
2-(4-((5-(3-(1H-pyrazol-1-yl)phenyl)-1H-tetrazol-1-yl)methyl)phenyl)-5-(difluoromethyl)-1,3,4-oxadiazole (compd. 289);
5-(1-((5-(5-(difluoromethyl)-1,3,4-oxadiazol-2-yl)pyrimidin-2-yl)methyl)-1H-tetrazol-5-yl)pyridin-2-amine (compd. 290);
5-(1-((5-(5-(difluoromethyl)-1,3,4-oxadiazol-2-yl)pyrimidin-2-yl)methyl)-1H-tetrazol-5-yl)-1-methyl-1H-benzo[d]imidazol-2-amine (compd. 291);
6-(1-((5-(5-(difluoromethyl)-1,3,4-oxadiazol-2-yl)pyridin-2-yl)methyl)-1H-imidazol-4-yl)isoindolin-1-one (compd. 292);
N-(3-(4-(6-aminopyridin-3-yl)-1H-1,2,3-triazol-1-yl)-3-(5-(5-(difluoromethyl)-1,3,4-oxadiazol-2-yl)pyridin-2-yl)propyl)methanesulfonamide (compd. 293);
6-(1-(4-(5-(difluoromethyl)-1,3,4-oxadiazol-2-yl)benzyl)-1H-imidazol-4-yl)isoindolin-1-one (compd. 294);
N-(3-(4-(6-aminopyridin-3-yl)-1H-1,2,3-triazol-1-yl)-3-(5-(5-(difluoromethyl)-1,3,4-oxadiazol-2-yl)pyridin-2-yl)propyl)-2,2-difluoroacetamide (compd. 295);
4-(1-(4-(5-(difluoromethyl)-1,3,4-oxadiazol-2-yl)-2,6-difluorobenzyl)-1H-1,2,3-triazol-4-yl)aniline (compd. 296);
3-(1-(4-(5-(difluoromethyl)-1,3,4-oxadiazol-2-yl)-2,6-difluorobenzyl)-1H-1,2,3-triazol-4-yl)aniline (compd. 297);
6-(1-((5-(5-(difluoromethyl)-1,3,4-oxadiazol-2-yl)pyrimidin-2-yl)methyl)-1H-tetrazol-5-yl)isoindolin-1-one (compd. 298);
2-(difluoromethyl)-5-(2-((5-(thiophen-2-yl)-1H-tetrazol-1-yl)methyl)pyrimidin-5-yl)-1,3,4-oxadiazole (compd. 299);
5-(1-(4-(5-(difluoromethyl)-1,3,4-oxadiazol-2-yl)benzyl)-1H-imidazol-4-yl)benzo[d]thiazol-2-amine (compd. 300);
5-(1-(1-(5-(5-(difluoromethyl)-1,3,4-oxadiazol-2-yl)pyridin-2-yl)-2-(pyrrolidin-1-yl)ethyl)-1H-1,2,3-triazol-4-yl)pyridin-2-amine (compd. 301).
Also the following compounds of formula (I) are preferred:
N-[2-[4-(6-aminopyridin-3-yl)triazol-1-yl]-2-[4-[5-(difluoromethyl)-1,3,4-oxadiazol-2-yl]phenyl]ethyl]methanesulfonamide compd. 302
5-[1-[1-[4-[5-(difluoromethyl)-1,3,4-oxadiazol-2-yl]phenyl]-4-piperidin-1-ylbutyl]triazol-4-yl]pyridin-2-amine compd. 303
5-[1-[[5-[5-(difluoromethyl)-1,3,4-oxadiazol-2-yl]-3-fluoropyridin-2-yl]methyl]triazol-4-yl]pyridin-2-amine compd. 304
3-[1-[[5-[5-(difluoromethyl)-1,3,4-oxadiazol-2-yl]pyridin-2-yl]methyl]imidazol-4-yl]benzamide compd. 305
6-[1-[[4-[5-(difluoromethyl)-1,3,4-oxadiazol-2-yl]phenyl]methyl]imidazol-4-yl]-1,3-benzothiazol-2-amine compd. 306
6-[1-[[5-[5-(difluoromethyl)-1,3,4-oxadiazol-2-yl]pyridin-2-yl]methyl]imidazol-4-yl]-1,3-benzothiazol-2-amine compd. 307
5-[1-[[5-[5-(difluoromethyl)-1,3,4-oxadiazol-2-yl]pyridin-2-yl]methyl]imidazol-4-yl]-1,3-benzoxazol-2-amine compd. 308
5-[1-[[4-[5-(difluoromethyl)-1,3,4-oxadiazol-2-yl]phenyl]methyl]imidazol-4-yl]-1,3-benzoxazol-2-amine compd. 309
N-[(3S)-3-[4-(6-aminopyridin-3-yl)triazol-1-yl]-3-[4-[5-(difluoromethyl)-1,3,4-oxadiazol-2-yl]phenyl]propyl]methanesulfonamide compd. 310
N-[(3R)-3-[4-(6-aminopyridin-3-yl)triazol-1-yl]-3-[4-[5-(difluoromethyl)-1,3,4-oxadiazol-2-yl]phenyl]propyl]methanesulfonamide compd. 311
5-[1-[(1R)-1-[4-[5-(difluoromethyl)-1,3,4-oxadiazol-2-yl]phenyl]-2-pyrrolidin-1-ylethyl]triazol-4-yl]pyridin-2-amine compd. 312
5-[1-[(1S)-1-[4-[5-(difluoromethyl)-1,3,4-oxadiazol-2-yl]phenyl]-2-pyrrolidin-1-ylethyl]triazol-4-yl]pyridin-2-amine compd. 313
(2R)-2-[4-(6-aminopyridin-3-yl)triazol-1-yl]-2-[4-[5-(difluoromethyl)-1,3,4-oxadiazol-2-yl]phenyl]ethanol compd. 314
4-[4-[[4-[5-(difluoromethyl)-1,3,4-oxadiazol-2-yl]phenyl]methyl]triazol-1-yl]aniline compd. 315
N-[4-[4-[[4-[5-(difluoromethyl)-1,3,4-oxadiazol-2-yl]phenyl]methyl]triazol-1-yl]phenyl]-4,5-dihydro-1H-imidazol-2-amine compd. 316
7-[1-[[5-[5-(difluoromethyl)-1,3,4-oxadiazol-2-yl]pyridin-2-yl]methyl]triazol-4-yl]quinazolin-4-amine compd. 317
6-[1-[[5-[5-(difluoromethyl)-1,3,4-oxadiazol-2-yl]pyridin-2-yl]methyl]pyrazol-4-yl]-2,3-dihydroisoindol-1-one compd. 318
6-[1-[[4-[5-(difluoromethyl)-1,3,4-oxadiazol-2-yl]phenyl]methyl]pyrazol-4-yl]-2,3-dihydroisoindol-1-one compd. 319
5-[1-[[5-[5-(difluoromethyl)-1,3,4-oxadiazol-2-yl]pyridin-2-yl]methyl]pyrazol-4-yl]-1-methylbenzimidazol-2-amine compd. 320
5-[1-[[4-[5-(difluoromethyl)-1,3,4-oxadiazol-2-yl]phenyl]methyl]pyrazol-4-yl]-1-methylbenzimidazol-2-amine compd. 321
5-[1-[[5-[5-(difluoromethyl)-1,3,4-oxadiazol-2-yl]pyridin-2-yl]methyl]imidazol-4-yl]-1,3-benzothiazol-2-amine compd. 322
5-[1-[1-[4-[5-(difluoromethyl)-1,3,4-oxadiazol-2-yl]phenyl]-3-pyrrolidin-1-ylpropyl]triazol-4-yl]pyridin-2-amine compd. 323
5-[1-[[5-[5-(difluoromethyl)-1,3,4-oxadiazol-2-yl]pyridin-2-yl]methyl]triazol-4-yl]-3,3-dimethyl-1H-indol-2-one compd. 324
5-[1-[[5-[5-(difluoromethyl)-1,3,4-oxadiazol-2-yl]pyridin-2-yl]methyl]triazol-4-yl]-1,3-dihydroindol-2-one compd. 325
6-[1-[[4-[5-(difluoromethyl)-1,3,4-oxadiazol-2-yl]phenyl]methyl]pyrazol-4-yl]-1,3-benzothiazol-2-amine compd. 326
6-[1-[[5-[5-(difluoromethyl)-1,3,4-oxadiazol-2-yl]pyridin-2-yl]methyl]pyrazol-4-yl]-1,3-benzothiazol-2-amine compd. 327
5-[1-[[5-[5-(difluoromethyl)-1,3,4-oxadiazol-2-yl]pyridin-2-yl]methyl]imidazol-4-yl]-1-methylbenzimidazol-2-amine compd. 328
5-[1-[[4-[5-(difluoromethyl)-1,3,4-oxadiazol-2-yl]phenyl]methyl]imidazol-4-yl]-1-methylbenzimidazol-2-amine compd. 329
4-[5-[[4-[5-(difluoromethyl)-1,3,4-oxadiazol-2-yl]phenyl]methyl]-1,3-oxazol-2-yl]aniline compd. 330
5-[1-[[5-[5-(difluoromethyl)-1,3,4-oxadiazol-2-yl]pyridin-2-yl]methyl]pyrazol-4-yl]-1H-benzimidazol-2-amine compd. 331
5-[1-[[4-[5-(difluoromethyl)-1,3,4-oxadiazol-2-yl]phenyl]methyl]pyrazol-4-yl]-1H-benzimidazol-2-amine compd. 332
3-[1-[[4-[5-(difluoromethyl)-1,3,4-oxadiazol-2-yl]phenyl]methyl]pyrazol-4-yl]benzamide compd. 333
3-[1-[[5-[5-(difluoromethyl)-1,3,4-oxadiazol-2-yl]pyridin-2-yl]methyl]pyrazol-4-yl]benzamide compd. 334
4-[4-(6-aminopyridin-3-yl)triazol-1-yl]-4-[4-[5-(difluoromethyl)-1,3,4-oxadiazol-2-yl]phenyl]butan-1-ol compd. 335
N-[3-[1-[[5-[5-(difluoromethyl)-1,3,4-oxadiazol-2-yl]-3-fluoropyridin-2-yl]methyl]triazol-4-yl]phenyl]morpholine-4-carboxamide compd. 336
N-[3-[1-[[2-chloro-4-[5-(difluoromethyl)-1,3,4-oxadiazol-2-yl]phenyl]methyl]triazol-4-yl]phenyl]morpholine-4-carboxamide compd. 337
N-[3-[1-[[4-[5-(difluoromethyl)-1,3,4-oxadiazol-2-yl]-2,3-difluorophenyl]methyl]triazol-4-yl]phenyl]morpholine-4-carboxamide compd. 338
6-[1-[[4-[5-(difluoromethyl)-1,3,4-oxadiazol-2-yl]-2-fluorophenyl]methyl]triazol-4-yl]-1,3-benzothiazol-2-amine compd. 339
6-[1-[[2-chloro-4-[5-(difluoromethyl)-1,3,4-oxadiazol-2-yl]phenyl]methyl]triazol-4-yl]-1,3-benzothiazol-2-amine compd. 340
6-[1-[[4-[5-(difluoromethyl)-1,3,4-oxadiazol-2-yl]-2,3-difluorophenyl]methyl]triazol-4-yl]-1,3-benzothiazol-2-amine compd. 341
6-[1-[[5-[5-(difluoromethyl)-1,3,4-oxadiazol-2-yl]-3-fluoropyridin-2-yl]methyl]triazol-4-yl]-1,3-benzothiazol-2-amine compd. 342
6-[1-[[4-[5-(difluoromethyl)-1,3,4-oxadiazol-2-yl]-2,5-difluorophenyl]methyl]triazol-4-yl]-1,3-benzothiazol-2-amine compd. 343
5-[1-[[2-chloro-4-[5-(difluoromethyl)-1,3,4-oxadiazol-2-yl]phenyl]methyl]triazol-4-yl]-1-methylbenzimidazol-2-amine compd. 344
5-[1-[[4-[5-(difluoromethyl)-1,3,4-oxadiazol-2-yl]-2,3-difluorophenyl]methyl]triazol-4-yl]-1-methylbenzimidazol-2-amine compd. 345
5-[1-[[4-[5-(difluoromethyl)-1,3,4-oxadiazol-2-yl]-3-fluorophenyl]methyl]triazol-4-yl]-1-methylbenzimidazol-2-amine compd. 346
6-[1-[[4-[5-(difluoromethyl)-1,3,4-oxadiazol-2-yl]-3-fluorophenyl]methyl]triazol-4-yl]-1,3-benzothiazol-2-amine compd. 347
5-[2-[[4-[5-(difluoromethyl)-1,3,4-oxadiazol-2-yl]-2-fluorophenyl]methyl]tetrazol-5-yl]-1-methylbenzimidazol-2-amine compd. 348
5-[1-[[5-[5-(difluoromethyl)-1,3,4-oxadiazol-2-yl]-3-fluoropyridin-2-yl]methyl]triazol-4-yl]-1-methylbenzimidazol-2-amine compd. 349
5-[1-[[4-[5-(difluoromethyl)-1,3,4-oxadiazol-2-yl]-2,5-difluorophenyl]methyl]triazol-4-yl]-1-methylbenzimidazol-2-amine compd. 350
6-[1-[dideuterio-[4-[5-(difluoromethyl)-1,3,4-oxadiazol-2-yl]phenyl]methyl]triazol-4-yl]-1,3-benzothiazol-2-amine compd. 351
N-[3-[1-[dideuterio-[4-[5-(difluoromethyl)-1,3,4-oxadiazol-2-yl]phenyl]methyl]triazol-4-yl]phenyl]morpholine-4-carboxamide compd. 352
2-(difluoromethyl)-5-[5-fluoro-6-[[5-[3-(4,5,6,7-tetrahydro-1H-imidazo[4,5-c]pyridin-2-yl)phenyl]tetrazol-2-yl]methyl]pyridin-3-yl]-1,3,4-oxadiazole compd. 353
2-(difluoromethyl)-5-[3-fluoro-4-[[5-[3-(4,5,6,7-tetrahydro-1H-imidazo[4,5-c]pyridin-2-yl)phenyl]tetrazol-2-yl]methyl]phenyl]-1,3,4-oxadiazole compd. 354
2-(difluoromethyl)-5-[2,3-difluoro-4-[[5-[3-(4,5,6,7-tetrahydro-1H-imidazo[4,5-c]pyridin-2-yl)phenyl]tetrazol-2-yl]methyl]phenyl]-1,3,4-oxadiazole compd. 355
5-[1-[dideuterio-[4-[5-(difluoromethyl)-1,3,4-oxadiazol-2-yl]phenyl]methyl]triazol-4-yl]pyridin-2-amine compd. 356
N-[4-[1-[[4-[5-(difluoromethyl)-1,3,4-oxadiazol-2-yl]-2-fluorophenyl]methyl]imidazol-4-yl]phenyl]-4,5-dihydro-1H-imidazol-2-amine compd. 357
N-[4-[1-[[2-chloro-4-[5-(difluoromethyl)-1,3,4-oxadiazol-2-yl]phenyl]methyl]imidazol-4-yl]phenyl]-4,5-dihydro-1H-imidazol-2-amine compd. 358
N-[4-[1-[[4-[5-(difluoromethyl)-1,3,4-oxadiazol-2-yl]-2,3-difluorophenyl]methyl]imidazol-4-yl]phenyl]-4,5-dihydro-1H-imidazol-2-amine compd. 359
N-[4-[1-[[4-[5-(difluoromethyl)-1,3,4-oxadiazol-2-yl]-3-fluorophenyl]methyl]imidazol-4-yl]phenyl]-4,5-dihydro-1H-imidazol-2-amine compd. 360
N-[4-[1-[[4-[5-(difluoromethyl)-1,3,4-oxadiazol-2-yl]-2,5-difluorophenyl]methyl]imidazol-4-yl]phenyl]-4,5-dihydro-1H-imidazol-2-amine compd. 361
5-[1-[dideuterio-[5-[5-(difluoromethyl)-1,3,4-oxadiazol-2-yl]pyridin-2-yl]methyl]triazol-4-yl]pyridin-2-amine compd. 362
N-[3-[1-[[4-[5-(difluoromethyl)-1,3,4-oxadiazol-2-yl]-2,5-difluorophenyl]methyl]triazol-4-yl]phenyl]morpholine-4-carboxamide compd. 363
2-(difluoromethyl)-5-[2-fluoro-4-[[5-[3-(4,5,6,7-tetrahydro-3H-imidazo[4,5-c]pyridin-2-yl)phenyl]tetrazol-2-yl]methyl]phenyl]-1,3,4-oxadiazole compd. 364
2-[3-chloro-4-[[5-[3-(4,5,6,7-tetrahydro-3H-imidazo[4,5-c]pyridin-2-yl)phenyl]tetrazol-2-yl]methyl]phenyl]-5-(difluoromethyl)-1,3,4-oxadiazole compd. 365
6-[5-[[4-[5-(difluoromethyl)-1,3,4-oxadiazol-2-yl]phenyl]methyl]-1,2-oxazol-3-yl]-1,3-benzothiazol-2-amine compd. 366
2-(difluoromethyl)-5-[2,5-difluoro-4-[[5-[3-(4,5,6,7-tetrahydro-1H-imidazo[4,5-c]pyridin-2-yl)phenyl]tetrazol-2-yl]methyl]phenyl]-1,3,4-oxadiazole compd. 367
N-[4-[1-[dideuterio-[4-[5-(difluoromethyl)-1,3,4-oxadiazol-2-yl]phenyl]methyl]imidazol-4-yl]phenyl]-4,5-dihydro-1H-imidazol-2-amine compd. 368
N-[3-[1-[dideuterio-[5-[5-(difluoromethyl)-1,3,4-oxadiazol-2-yl]pyridin-2-yl]methyl]triazol-4-yl]phenyl]morpholine-4-carboxamide compd. 369
5-[2-[[5-[5-(difluoromethyl)-1,3,4-oxadiazol-2-yl]-3-fluoropyridin-2-yl]methyl]tetrazol-5-yl]-1-methylbenzimidazol-2-amine compd. 370
5-[2-[[4-[5-(difluoromethyl)-1,3,4-oxadiazol-2-yl]-2,3-difluorophenyl]methyl]tetrazol-5-yl]-1-methylbenzimidazol-2-amine compd. 371
5-[2-[[4-[5-(difluoromethyl)-1,3,4-oxadiazol-2-yl]-2,5-difluorophenyl]methyl]tetrazol-5-yl]-1-methylbenzimidazol-2-amine compd. 372
5-[2-[[4-[5-(difluoromethyl)-1,3,4-oxadiazol-2-yl]-3-fluorophenyl]methyl]tetrazol-5-yl]-1-methylbenzimidazol-2-amine compd. 373
5-[2-[[2-chloro-4-[5-(difluoromethyl)-1,3,4-oxadiazol-2-yl]phenyl]methyl]tetrazol-5-yl]-1-methylbenzimidazol-2-amine compd. 374
4-[5-[[4-[5-(difluoromethyl)-1,3,4-oxadiazol-2-yl]phenyl]methyl]-1,2,4-oxadiazol-3-yl]aniline compd. 375
6-[1-[dideuterio-[5-[5-(difluoromethyl)-1,3,4-oxadiazol-2-yl]pyridin-2-yl]methyl]triazol-4-yl]-1,3-benzothiazol-2-amine compd. 376
6-[4-[[4-[5-(difluoromethyl)-1,3,4-oxadiazol-2-yl]phenyl]methyl]triazol-1-yl]-1,3-benzothiazol-2-amine compd. 377
5-[1-[[4-[5-(difluoromethyl)-1,3,4-oxadiazol-2-yl]-2-fluorophenyl]methyl]triazol-4-yl]-1-methylbenzimidazol-2-amine compd. 378
N-[4-[5-[[4-[5-(difluoromethyl)-1,3,4-oxadiazol-2-yl]phenyl]methyl]-1,2,4-oxadiazol-3-yl]phenyl]-4,5-dihydro-1H-imidazol-2-amine compd. 379
5-[1-[dideuterio-[4-[5-(difluoromethyl)-1,3,4-oxadiazol-2-yl]-2,5-difluorophenyl]methyl]triazol-4-yl]pyridin-2-amine compd. 381
6-[1-[dideuterio-[4-[5-(difluoromethyl)-1,3,4-oxadiazol-2-yl]-2-fluorophenyl]methyl]triazol-4-yl]-1,3-benzothiazol-2-amine compd. 382
N-(4-(5-(4-(5-(difluoromethyl)-1,3,4-oxadiazol-2-yl)-3-fluorobenzyl)-1,2,4-oxadiazol-3-yl)phenyl)-4,5-dihydro-1H-imidazol-2-amine compd. 383
6-(1-(4-(5-(difluoromethyl)-1,3,4-oxadiazol-2-yl)-2,3-difluorobenzyl)-1H-1,2,3-triazol-4-yl)thieno[2,3-d]pyrimidin-4-amine compd. 384
5-[1-[dideuterio-[4-[5-(difluoromethyl)-1,3,4-oxadiazol-2-yl]-2,3-difluorophenyl]methyl]triazol-4-yl]pyridin-2-amine compd. 385
6-(1-(4-(5-(difluoromethyl)-1,3,4-oxadiazol-2-yl)-2,6-difluorobenzyl)-1H-1,2,3-triazol-4-yl)thieno[2,3-d]pyrimidin-4-amine compd. 386
7-[1-({4-[5-(difluoromethyl)-1,3,4-oxadiazol-2-yl]-2-fluorophenyl}methyl)-1H-1,2,3-triazol-4-yl]quinazolin-4-amine compd. 387
6-(2-(4-(5-(difluoromethyl)-1,3,4-oxadiazol-2-yl)-2,6-difluorobenzyl)-2H-tetrazol-5-yl)-N-methylquinolin-2-amine compd. 388
6-[1-({4-[5-(difluoromethyl)-1,3,4-oxadiazol-2-yl]-2,3-difluorophenyl}methyl)-1H-1,2,3-triazol-4-yl]-N-methylquinazolin-2-amine compd. 389
6-[1-({4-[5-(difluoromethyl)-1,3,4-oxadiazol-2-yl]-2-fluorophenyl}methyl)-1H-1,2,3-triazol-4-yl]-N-methylquinazolin-2-amine compd. 390
6-[1-({4-[5-(difluoromethyl)-1,3,4-oxadiazol-2-yl]-2,5-difluorophenyl}methyl)-1H-1,2,3-triazol-4-yl]-N-methylquinazolin-2-amine compd. 391
6-[1-({4-[5-(difluoromethyl)-1,3,4-oxadiazol-2-yl]-2,6-difluorophenyl}methyl)-1H-1,2,3-triazol-4-yl]-N-methylquinazolin-2-amine compd. 392
6-[1-({4-[5-(difluoromethyl)-1,3,4-oxadiazol-2-yl]-3-fluorophenyl}methyl)-1H-1,2,3-triazol-4-yl]-N-methylquinazolin-2-amine compd. 393
6-[1-({4-[5-(difluoromethyl)-1,3,4-oxadiazol-2-yl]-2,5-difluorophenyl}methyl)-1H-1,2,3-triazol-4-yl]-N-ethylquinazolin-2-amine compd. 394
6-(1-(4-(5-(difluoromethyl)-1,3,4-oxadiazol-2-yl)-3-fluorobenzyl)-1H-1,2,3-triazol-4-yl)-N-ethylquinazolin-2-amine compd. 395
6-[2-[[4-[5-(difluoromethyl)-1,3,4-oxadiazol-2-yl]-2,5-difluorophenyl]methyl]tetrazol-5-yl]isoquinolin-1-amine compd. 396
6-[2-[[4-[5-(difluoromethyl)-1,3,4-oxadiazol-2-yl]-2-fluorophenyl]methyl]tetrazol-5-yl]isoquinolin-1-amine compd. 397
6-[2-[[4-[5-(difluoromethyl)-1,3,4-oxadiazol-2-yl]-2,5-difluorophenyl]methyl]tetrazol-5-yl]quinolin-3-amine compd. 398
6-(1-(4-(5-(difluoromethyl)-1,3,4-oxadiazol-2-yl)-2,6-difluorobenzyl)-1H-1,2,3-triazol-4-yl)-N,N-dimethylquinolin-2-amine compd. 399
6-[2-[[4-[5-(difluoromethyl)-1,3,4-oxadiazol-2-yl]-3-fluorophenyl]methyl]tetrazol-5-yl]quinolin-3-amine compd. 400
6-[2-[[4-[5-(difluoromethyl)-1,3,4-oxadiazol-2-yl]-2-fluorophenyl]methyl]tetrazol-5-yl]-N-methylquinolin-2-amine compd. 401
6-[1-({4-[5-(difluoromethyl)-1,3,4-oxadiazol-2-yl]-3-fluorophenyl}methyl)-1H-1,2,3-triazol-4-yl]-N,N-dimethylquinazolin-2-amine compd. 402
6-[2-[[4-[5-(difluoromethyl)-1,3,4-oxadiazol-2-yl]-3-fluorophenyl]methyl]tetrazol-5-yl]-N-methylquinolin-2-amine compd. 403
6-(1-(2-chloro-4-(5-(difluoromethyl)-1,3,4-oxadiazol-2-yl)benzyl)-1H-1,2,3-triazol-4-yl)isoquinolin-3-amine compd. 404
6-[1-({4-[5-(difluoromethyl)-1,3,4-oxadiazol-2-yl]-2-fluorophenyl}methyl)-1H-1,2,3-triazol-4-yl]isoquinolin-3-amine compd. 405
6-[1-({4-[5-(difluoromethyl)-1,3,4-oxadiazol-2-yl]-2,5-difluorophenyl}methyl)-1H-1,2,3-triazol-4-yl]isoquinolin-3-amine compd. 406
6-(2-(4-(5-(difluoromethyl)-1,3,4-oxadiazol-2-yl)-2,3-difluorobenzyl)-2H-tetrazol-5-yl)-N-methylquinolin-2-amine compd. 407
4-(5-(4-(5-(difluoromethyl)-1,3,4-oxadiazol-2-yl)-3-fluorobenzyl)-1,2,4-oxadiazol-3-yl)aniline compd. 408
6-(2-(4-(5-(difluoromethyl)-1,3,4-oxadiazol-2-yl)-2,5-difluorobenzyl)-2H-tetrazol-5-yl)-N-ethylquinolin-2-amine compd. 409
6-(2-(4-(5-(difluoromethyl)-1,3,4-oxadiazol-2-yl)-3-fluorobenzyl)-2H-tetrazol-5-yl)-N-ethylquinolin-2-amine compd. 410
5-(4-(4-(5-(difluoromethyl)-1,3,4-oxadiazol-2-yl)benzyl)-1H-1,2,3-triazol-1-yl)pyridin-2-amine compd. 413
5-[4-({4-[5-(difluoromethyl)-1,3,4-oxadiazol-2-yl]phenyl}methyl)-1H-1,2,3-triazol-1-yl]-1-methyl-1H-1,3-benzodiazol-2-amine compd. 414
6-[1-({4-[5-(difluoromethyl)-1,3,4-oxadiazol-2-yl]-2,5-difluorophenyl}methyl)-1H-1,2,3-triazol-4-yl]thieno[2,3-d]pyrimidin-4-amine compd. 415
6-[1-({4-[5-(difluoromethyl)-1,3,4-oxadiazol-2-yl]-2-fluorophenyl}methyl)-1H-1,2,3-triazol-4-yl]thieno[2,3-d]pyrimidin-4-amine compd. 416
6-[1-({4-[5-(difluoromethyl)-1,3,4-oxadiazol-2-yl]-3-fluorophenyl}methyl)-1H-1,2,3-triazol-4-yl]thieno[2,3-d]pyrimidin-4-amine compd. 417
7-(1-(4-(5-(difluoromethyl)-1,3,4-oxadiazol-2-yl)-3-fluorobenzyl)-1H-1,2,3-triazol-4-yl)quinazolin-4-amine compd. 418
4-(5-(4-(5-(difluoromethyl)-1,3,4-oxadiazol-2-yl)-2-fluorobenzyl)-1,2,4-oxadiazol-3-yl)aniline compd. 419
N-(4-(5-(4-(5-(difluoromethyl)-1,3,4-oxadiazol-2-yl)-2-fluorobenzyl)-1,2,4-oxadiazol-3-yl)phenyl)-4,5-dihydro-1H-imidazol-2-amine compd. 420
6-(2-(2-chloro-4-(5-(difluoromethyl)-1,3,4-oxadiazol-2-yl)benzyl)-2H-tetrazol-5-yl)isoquinolin-1-amine compd. 422
6-(2-(4-(5-(difluoromethyl)-1,3,4-oxadiazol-2-yl)-2,5-difluorobenzyl)-2H-tetrazol-5-yl)quinazolin-2-amine compd. 423
6-(2-(4-(5-(difluoromethyl)-1,3,4-oxadiazol-2-yl)-2-fluorobenzyl)-2H-tetrazol-5-yl)quinazolin-2-amine compd. 424
6-(2-(2-chloro-4-(5-(difluoromethyl)-1,3,4-oxadiazol-2-yl)benzyl)-2H-tetrazol-5-yl)quinazolin-2-amine compd. 425
2-(3-chloro-4-((5-(isoquinolin-6-yl)-2H-tetrazol-2-yl)methyl)phenyl)-5-(difluoromethyl)-1,3,4-oxadiazole compd. 426
2-(difluoromethyl)-5-(3-fluoro-4-((5-(isoquinolin-6-yl)-2H-tetrazol-2-yl)methyl)phenyl)-1,3,4-oxadiazole compd. 427
2-(2,5-difluoro-4-((5-(isoquinolin-6-yl)-2H-tetrazol-2-yl)methyl)phenyl)-5-(difluoromethyl)-1,3,4-oxadiazole compd. 428
6-(2-(2-chloro-4-(5-(difluoromethyl)-1,3,4-oxadiazol-2-yl)benzyl)-2H-tetrazol-5-yl)quinolin-3-amine compd. 429
2-(3-chloro-4-((5-(isoquinolin-1-yl)-2H-tetrazol-2-yl)methyl)phenyl)-5-(difluoromethyl)-1,3,4-oxadiazole compd. 430
2-(difluoromethyl)-5-(3-fluoro-4-((5-(isoquinolin-1-yl)-2H-tetrazol-2-yl)methyl)phenyl)-1,3,4-oxadiazole compd. 431
2-(2,5-difluoro-4-((5-(isoquinolin-1-yl)-2H-tetrazol-2-yl)methyl)phenyl)-5-(difluoromethyl)-1,3,4-oxadiazole compd. 432
7-(1-(4-(5-(difluoromethyl)-1,3,4-oxadiazol-2-yl)-2,5-difluorobenzyl)-1H-1,2,3-triazol-4-yl)quinazolin-4-amine compd. 433
7-(1-(2-chloro-4-(5-(difluoromethyl)-1,3,4-oxadiazol-2-yl)benzyl)-1H-1,2,3-triazol-4-yl)quinazolin-4-amine compd. 434
2-(difluoromethyl)-5-[3-fluoro-4-[[5-(1-pyrazin-2-ylcyclopropyl)tetrazol-2-yl]methyl]phenyl]-1,3,4-oxadiazole compd. 435
2-(difluoromethyl)-5-[2-fluoro-4-[[5-(1-pyrazin-2-ylcyclopropyl)tetrazol-2-yl]methyl]phenyl]-1,3,4-oxadiazole compd. 436
2-(difluoromethyl)-5-[2,3-difluoro-4-[[5-(1-pyrazin-2-ylcyclopropyl)tetrazol-2-yl]methyl]phenyl]-1,3,4-oxadiazole compd. 437
2-(difluoromethyl)-5-[2,5-difluoro-4-[[5-(1-pyrazin-2-ylcyclopropyl)tetrazol-2-yl]methyl]phenyl]-1,3,4-oxadiazole compd. 438
2-(difluoromethyl)-5-[3,5-difluoro-4-[[5-(1-pyrazin-2-ylcyclopropyl)tetrazol-2-yl]methyl]phenyl]-1,3,4-oxadiazole compd. 439
2-[3-chloro-4-[[5-(1-pyrazin-2-ylcyclopropyl)tetrazol-2-yl]methyl]phenyl]-5-(difluoromethyl)-1,3,4-oxadiazole compd. 440
6-[2-[2-[[4-[5-(difluoromethyl)-1,3,4-oxadiazol-2-yl]-2-fluorophenyl]methyl]tetrazol-5-yl]propan-2-yl]pyridin-3-amine compd. 441
6-[2-[2-[[4-[5-(difluoromethyl)-1,3,4-oxadiazol-2-yl]-3-fluorophenyl]methyl]tetrazol-5-yl]propan-2-yl]pyridin-3-amine compd. 442
6-[2-[2-[[4-[5-(difluoromethyl)-1,3,4-oxadiazol-2-yl]-2,3-difluorophenyl]methyl]tetrazol-5-yl]propan-2-yl]pyridin-3-amine compd. 443
6-[2-[2-[[4-[5-(difluoromethyl)-1,3,4-oxadiazol-2-yl]-2,5-difluorophenyl]methyl]tetrazol-5-yl]propan-2-yl]pyridin-3-amine compd. 444
6-[2-[2-[[4-[5-(difluoromethyl)-1,3,4-oxadiazol-2-yl]-2,6-difluorophenyl]methyl]tetrazol-5-yl]propan-2-yl]pyridin-3-amine compd. 445
6-[2-[2-[[2-chloro-4-[5-(difluoromethyl)-1,3,4-oxadiazol-2-yl]phenyl]methyl]tetrazol-5-yl]propan-2-yl]pyridin-3-amine compd. 446
2-[2-[2-[[4-[5-(difluoromethyl)-1,3,4-oxadiazol-2-yl]-2-fluorophenyl]methyl]tetrazol-5-yl]propan-2-yl]pyridin-4-amine compd. 447
2-[2-[2-[[4-[5-(difluoromethyl)-1,3,4-oxadiazol-2-yl]-3-fluorophenyl]methyl]tetrazol-5-yl]propan-2-yl]pyridin-4-amine compd. 448
2-[2-[2-[[4-[5-(difluoromethyl)-1,3,4-oxadiazol-2-yl]-2,3-difluorophenyl]methyl]tetrazol-5-yl]propan-2-yl]pyridin-4-amine compd. 449
2-[2-[2-[[4-[5-(difluoromethyl)-1,3,4-oxadiazol-2-yl]-2,5-difluorophenyl]methyl]tetrazol-5-yl]propan-2-yl]pyridin-4-amine compd. 450
2-[2-[2-[[4-[5-(difluoromethyl)-1,3,4-oxadiazol-2-yl]-2,6-difluorophenyl]methyl]tetrazol-5-yl]propan-2-yl]pyridin-4-amine compd. 451
2-[2-[2-[[2-chloro-4-[5-(difluoromethyl)-1,3,4-oxadiazol-2-yl]phenyl]methyl]tetrazol-5-yl]propan-2-yl]pyridin-4-amine compd. 452
2-[2-[[4-[5-(difluoromethyl)-1,3,4-oxadiazol-2-yl]-2-fluorophenyl]methyl]tetrazol-5-yl]pyrimidin-5-amine compd. 453
2-[2-[[4-[5-(difluoromethyl)-1,3,4-oxadiazol-2-yl]-3-fluorophenyl]methyl]tetrazol-5-yl]pyrimidin-5-amine compd. 454
2-[2-[[4-[5-(difluoromethyl)-1,3,4-oxadiazol-2-yl]-2,3-difluorophenyl]methyl]tetrazol-5-yl]pyrimidin-5-amine compd. 455
2-[2-[[4-[5-(difluoromethyl)-1,3,4-oxadiazol-2-yl]-2,5-difluorophenyl]methyl]tetrazol-5-yl]pyrimidin-5-amine compd. 456
2-[2-[[4-[5-(difluoromethyl)-1,3,4-oxadiazol-2-yl]-2,6-difluorophenyl]methyl]tetrazol-5-yl]pyrimidin-5-amine compd. 457
2-[2-[[2-chloro-4-[5-(difluoromethyl)-1,3,4-oxadiazol-2-yl]phenyl]methyl]tetrazol-5-yl]pyrimidin-5-amine compd. 458
6-[2-[[4-[5-(difluoromethyl)-1,3,4-oxadiazol-2-yl]-2,3-difluorophenyl]methyl]tetrazol-5-yl]isoquinolin-1-amine compd. 459
6-[2-[[4-[5-(difluoromethyl)-1,3,4-oxadiazol-2-yl]-2,6-difluorophenyl]methyl]tetrazol-5-yl]isoquinolin-1-amine compd. 460
6-[2-[[4-[5-(difluoromethyl)-1,3,4-oxadiazol-2-yl]-2,6-difluorophenyl]methyl]tetrazol-5-yl]quinolin-3-amine compd. 461
6-[2-[[4-[5-(difluoromethyl)-1,3,4-oxadiazol-2-yl]-2,3-difluorophenyl]methyl]tetrazol-5-yl]quinolin-3-amine compd. 462
6-[2-[[4-[5-(difluoromethyl)-1,3,4-oxadiazol-2-yl]-3-fluorophenyl]methyl]tetrazol-5-yl]quinazolin-2-amine compd. 463
6-[2-[[4-[5-(difluoromethyl)-1,3,4-oxadiazol-2-yl]-2,3-difluorophenyl]methyl]tetrazol-5-yl]quinazolin-2-amine compd. 464
6-[2-[[4-[5-(difluoromethyl)-1,3,4-oxadiazol-2-yl]-2,6-difluorophenyl]methyl]tetrazol-5-yl]quinazolin-2-amine compd. 465
2-(difluoromethyl)-5-[2-fluoro-4-[(5-isoquinolin-6-yltetrazol-2-yl)methyl]phenyl]-1,3,4-oxadiazole compd. 466
2-[2,3-difluoro-4-[(5-isoquinolin-6-yltetrazol-2-yl)methyl]phenyl]-5-(difluoromethyl)-1,3,4-oxadiazole compd. 467
2-[3,5-difluoro-4-[(5-isoquinolin-6-yltetrazol-2-yl)methyl]phenyl]-5-(difluoromethyl)-1,3,4-oxadiazole compd. 468
2-(difluoromethyl)-5-[2-fluoro-4-[(5-isoquinolin-1-yltetrazol-2-yl)methyl]phenyl]-1,3,4-oxadiazole compd. 469
2-[2,3-difluoro-4-[(5-isoquinolin-1-yltetrazol-2-yl)methyl]phenyl]-5-(difluoromethyl)-1,3,4-oxadiazole compd. 470
2-[3,5-difluoro-4-[(5-isoquinolin-1-yltetrazol-2-yl)methyl]phenyl]-5-(difluoromethyl)-1,3,4-oxadiazole compd. 471
6-[2-[[2-chloro-4-[5-(difluoromethyl)-1,3,4-oxadiazol-2-yl]phenyl]methyl]tetrazol-5-yl]-N-methylquinolin-2-amine compd. 472
6-[2-[[4-[5-(difluoromethyl)-1,3,4-oxadiazol-2-yl]-2-fluorophenyl]methyl]tetrazol-5-yl]-N-ethylquinolin-2-amine compd. 473
6-[2-[[4-[5-(difluoromethyl)-1,3,4-oxadiazol-2-yl]-2,3-difluorophenyl]methyl]tetrazol-5-yl]-N-ethylquinolin-2-amine compd. 474
6-[2-[[4-[5-(difluoromethyl)-1,3,4-oxadiazol-2-yl]-2,6-difluorophenyl]methyl]tetrazol-5-yl]-N-ethylquinolin-2-amine compd. 475
6-[2-[[2-chloro-4-[5-(difluoromethyl)-1,3,4-oxadiazol-2-yl]phenyl]methyl]tetrazol-5-yl]-N-ethylquinolin-2-amine compd. 476
6-[1-[(1R)-1-[4-[5-(difluoromethyl)-1,3,4-oxadiazol-2-yl]-2-fluorophenyl]ethyl]triazol-4-yl]-1,3-benzothiazol-2-amine compd. 477
6-[1-[(1R)-1-[2-chloro-4-[5-(difluoromethyl)-1,3,4-oxadiazol-2-yl]phenyl]ethyl]triazol-4-yl]-1,3-benzothiazol-2-amine compd. 478
6-[1-[(1R)-1-[4-[5-(difluoromethyl)-1,3,4-oxadiazol-2-yl]-3-fluorophenyl]ethyl]triazol-4-yl]-1,3-benzothiazol-2-amine compd. 479
6-[1-[(1R)-1-[4-[5-(difluoromethyl)-1,3,4-oxadiazol-2-yl]-2,3-difluorophenyl]ethyl]triazol-4-yl]-1,3-benzothiazol-2-amine compd. 480
6-[1-[(1R)-1-[4-[5-(difluoromethyl)-1,3,4-oxadiazol-2-yl]-2,5-difluorophenyl]ethyl]triazol-4-yl]-1,3-benzothiazol-2-amine compd. 481
6-[1-[(1R)-1-[4-[5-(difluoromethyl)-1,3,4-oxadiazol-2-yl]-2,6-difluorophenyl]ethyl]triazol-4-yl]-1,3-benzothiazol-2-amine compd. 482
6-[2-[[4-[5-(difluoromethyl)-1,3,4-oxadiazol-2-yl]-2,5-difluorophenyl]methyl]tetrazol-5-yl]-N-methylquinazolin-2-amine compd. 483
6-[2-[[4-[5-(difluoromethyl)-1,3,4-oxadiazol-2-yl]-3-fluorophenyl]methyl]tetrazol-5-yl]-N-methylquinazolin-2-amine compd. 484
6-[2-[[4-[5-(difluoromethyl)-1,3,4-oxadiazol-2-yl]-2-fluorophenyl]methyl]tetrazol-5-yl]-N-methylquinazolin-2-amine compd. 485
6-[2-[[4-[5-(difluoromethyl)-1,3,4-oxadiazol-2-yl]-2,5-difluorophenyl]methyl]tetrazol-5-yl]-N,N-dimethylquinazolin-2-amine compd. 486
6-[2-[[4-[5-(difluoromethyl)-1,3,4-oxadiazol-2-yl]-3-fluorophenyl]methyl]tetrazol-5-yl]-N,N-dimethylquinazolin-2-amine compd. 487
6-[2-[[4-[5-(difluoromethyl)-1,3,4-oxadiazol-2-yl]-2-fluorophenyl]methyl]tetrazol-5-yl]-N,N-dimethylquinazolin-2-amine compd. 488
6-[1-[[2-chloro-4-[5-(difluoromethyl)-1,3,4-oxadiazol-2-yl]phenyl]methyl]triazol-4-yl]-N-methylquinazolin-2-amine compd. 489
6-[1-[[4-[5-(difluoromethyl)-1,3,4-oxadiazol-2-yl]-2,3-difluorophenyl]methyl]triazol-4-yl]-N-ethylquinazolin-2-amine compd. 490
6-[1-[[4-[5-(difluoromethyl)-1,3,4-oxadiazol-2-yl]-2,6-difluorophenyl]methyl]triazol-4-yl]-N-ethylquinazolin-2-amine compd. 491
6-[1-[[2-chloro-4-[5-(difluoromethyl)-1,3,4-oxadiazol-2-yl]phenyl]methyl]triazol-4-yl]-N-ethylquinazolin-2-amine compd. 492
6-[1-[[4-[5-(difluoromethyl)-1,3,4-oxadiazol-2-yl]-2,5-difluorophenyl]methyl]triazol-4-yl]-N,N-dimethylquinazolin-2-amine compd. 494
6-[1-[[4-[5-(difluoromethyl)-1,3,4-oxadiazol-2-yl]-2,3-difluorophenyl]methyl]triazol-4-yl]-N,N-dimethylquinazolin-2-amine compd. 495
6-[1-[[4-[5-(difluoromethyl)-1,3,4-oxadiazol-2-yl]-2-fluorophenyl]methyl]triazol-4-yl]-N,N-dimethylquinazolin-2-amine compd. 496
6-[1-[[2-chloro-4-[5-(difluoromethyl)-1,3,4-oxadiazol-2-yl]phenyl]methyl]triazol-4-yl]-N,N-dimethylquinazolin-2-amine compd. 497
6-[1-[[4-[5-(difluoromethyl)-1,3,4-oxadiazol-2-yl]-2,3-difluorophenyl]methyl]triazol-4-yl]isoquinolin-3-amine compd. 498
6-[1-[[4-[5-(difluoromethyl)-1,3,4-oxadiazol-2-yl]-2,6-difluorophenyl]methyl]triazol-4-yl]isoquinolin-3-amine compd. 499
6-[1-[[4-[5-(difluoromethyl)-1,3,4-oxadiazol-2-yl]-3-fluorophenyl]methyl]triazol-4-yl]isoquinolin-3-amine compd. 500
6-[1-[[2-chloro-4-[5-(difluoromethyl)-1,3,4-oxadiazol-2-yl]phenyl]methyl]triazol-4-yl]thieno[2,3-d]pyrimidin-4-amine compd. 501
7-[1-[[4-[5-(difluoromethyl)-1,3,4-oxadiazol-2-yl]-2,3-difluorophenyl]methyl]triazol-4-yl]quinazolin-4-amine compd. 502
7-[1-[[4-[5-(difluoromethyl)-1,3,4-oxadiazol-2-yl]-2,6-difluorophenyl]methyl]triazol-4-yl]quinazolin-4-amine compd. 503
6-(2-(4-(5-(difluoromethyl)-1,3,4-oxadiazol-2-yl)-2,5-difluorobenzyl)-2H-tetrazol-5-yl)-N-methylquinolin-2-amine compd. 504
5-[1-[dideuterio-[4-[5-(difluoromethyl)-1,3,4-oxadiazol-2-yl]-3-fluorophenyl]methyl]triazol-4-yl]pyridin-2-amine compd. 505
5-[1-[dideuterio-[4-[5-(difluoromethyl)-1,3,4-oxadiazol-2-yl]-2-fluorophenyl]methyl]triazol-4-yl]pyridin-2-amine compd. 506
6-[1-[dideuterio-[4-[5-(difluoromethyl)-1,3,4-oxadiazol-2-yl]-3-fluorophenyl]methyl]triazol-4-yl]-1,3-benzothiazol-2-amine compd. 507
6-[1-[dideuterio-[4-[5-(difluoromethyl)-1,3,4-oxadiazol-2-yl]-2,5-difluorophenyl]methyl]triazol-4-yl]-1,3-benzothiazol-2-amine compd. 508
6-[1-[dideuterio-[4-[5-(difluoromethyl)-1,3,4-oxadiazol-2-yl]-2,3-difluorophenyl]methyl]triazol-4-yl]-1,3-benzothiazol-2-amine compd. 509
5-[1-[dideuterio-[4-[5-(difluoromethyl)-1,3,4-oxadiazol-2-yl]-3-fluorophenyl]methyl]triazol-4-yl]-1-methylbenzimidazol-2-amine compd. 510
5-[1-[dideuterio-[4-[5-(difluoromethyl)-1,3,4-oxadiazol-2-yl]-2-fluorophenyl]methyl]triazol-4-yl]-1-methylbenzimidazol-2-amine compd. 511
5-[1-[dideuterio-[4-[5-(difluoromethyl)-1,3,4-oxadiazol-2-yl]-2,5-difluorophenyl]methyl]triazol-4-yl]-1-methylbenzimidazol-2-amine compd. 512
5-[1-[dideuterio-[4-[5-(difluoromethyl)-1,3,4-oxadiazol-2-yl]-2,3-difluorophenyl]methyl]triazol-4-yl]-1-methylbenzimidazol-2-amine compd. 513
6-[5-[[4-[5-(difluoromethyl)-1,3,4-oxadiazol-2-yl]phenyl]methyl]-1,2,4-oxadiazol-3-yl]-1,3-benzothiazol-2-amine compd. 514
6-[2-[[4-[5-(difluoromethyl)-1,3,4-oxadiazol-2-yl]-3-fluorophenyl]methyl]tetrazol-5-yl]isoquinolin-1-amine compd. 515
6-[2-[[4-[5-(difluoromethyl)-1,3,4-oxadiazol-2-yl]-2-fluorophenyl]methyl]tetrazol-5-yl]quinolin-3-amine compd. 516
6-[2-[[4-[5-(difluoromethyl)-1,3,4-oxadiazol-2-yl]-2,5-difluorophenyl]methyl]tetrazol-5-yl]-N-methylquinolin-2-amine compd. 517
6-[2-[[4-[5-(difluoromethyl)-1,3,4-oxadiazol-2-yl]-2,5-difluorophenyl]methyl]tetrazol-5-yl]isoquinolin-3-amine compd. 518
6-[2-[[4-[5-(difluoromethyl)-1,3,4-oxadiazol-2-yl]-2-fluorophenyl]methyl]tetrazol-5-yl]isoquinolin-3-amine compd. 519
6-[2-[[4-[5-(difluoromethyl)-1,3,4-oxadiazol-2-yl]-3-fluorophenyl]methyl]tetrazol-5-yl]isoquinolin-3-amine compd. 520
7-[2-[[4-[5-(difluoromethyl)-1,3,4-oxadiazol-2-yl]-2,5-difluorophenyl]methyl]tetrazol-5-yl]quinazolin-4-amine compd. 521
7-[2-[[4-[5-(difluoromethyl)-1,3,4-oxadiazol-2-yl]-2-fluorophenyl]methyl]tetrazol-5-yl]quinazolin-4-amine compd. 522
7-[2-[[4-[5-(difluoromethyl)-1,3,4-oxadiazol-2-yl]-3-fluorophenyl]methyl]tetrazol-5-yl]quinazolin-4-amine compd. 523
6-[2-[[4-[5-(difluoromethyl)-1,3,4-oxadiazol-2-yl]-3-fluorophenyl]methyl]tetrazol-5-yl]thieno[2,3-d]pyrimidin-4-amine compd. 524
6-[2-[[4-[5-(difluoromethyl)-1,3,4-oxadiazol-2-yl]-2,5-difluorophenyl]methyl]tetrazol-5-yl]thieno[2,3-d]pyrimidin-4-amine compd. 525
6-[2-[[4-[5-(difluoromethyl)-1,3,4-oxadiazol-2-yl]-2-fluorophenyl]methyl]tetrazol-5-yl]thieno[2,3-d]pyrimidin-4-amine compd. 526.

A further class of preferred compounds comprises compounds of formula (I) and pharmaceutically acceptable salts, isomers and prodrugs thereof, wherein the pentaheterocyclic core A-B-D-E-M is selected from the group consisting of 1,2,3-triazole, 2,5-disubstituted tetrazole, 1,4-disubstituted pyrazole, imidazole, 1,3,4-thiadiazole, 1,2,4-oxadiazole, 1,3,4-oxadiazole and isoxazole. Preferably, the pentaheterocyclic core A-B-D-E-M is selected from the group consisting of 1,2,3-triazole wherein B═C and M=N, 2,5-disubstituted tetrazole, 1,4-disubstituted pyrazole, 1,3,4-thiadiazole, 1,2,4-oxadiazole, 1,3,4-oxadiazole and isoxazole. More preferably, the pentaheterocyclic core A-B-D-E-M is selected from the group consisting of 1,2,3-triazole wherein B═C and M=N, 1,3,4-thiadiazole, 1,2,4-oxadiazole, 1,3,4-oxadiazole and isoxazole. Another class of preferred compounds comprises compounds of formula (I) and pharmaceutically acceptable salts, isomers and prodrugs thereof, wherein at least one among X, X′, Y and Y′ is CF or at least one between X and X′ is CCl.

Another class of preferred compounds comprises compounds of formula (I) and pharmaceutically acceptable salts, isomers and prodrugs thereof, wherein Z═—CD₂-, —CF₂—, —CHR³—, —NH—, —S—;

wherein R³ is selected among the following substructures:

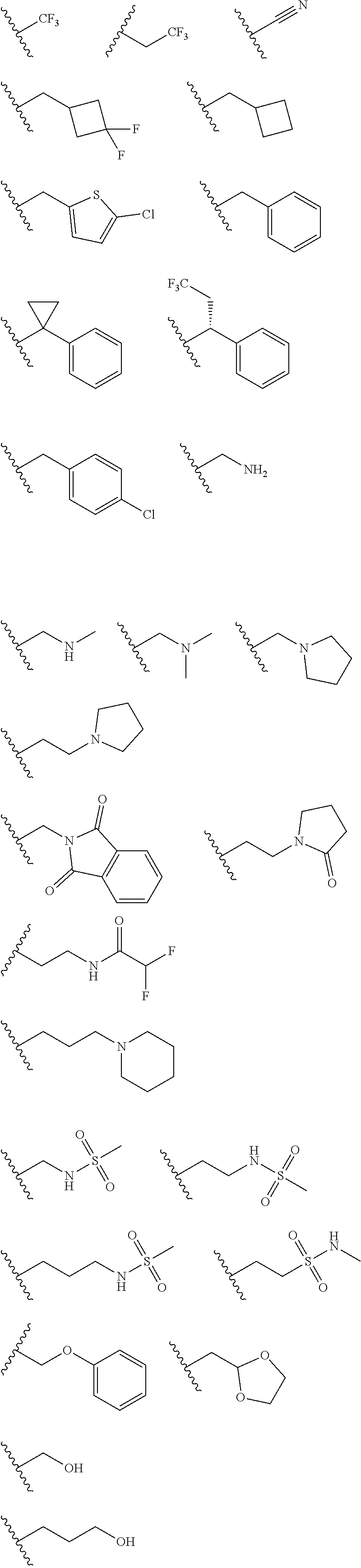

More preferably, Z═—CD₂-, —CF₂, —CHR³—, —S—

wherein R³ is selected among the following substructures:

Another class of preferred compounds comprises compounds of formula (I) and

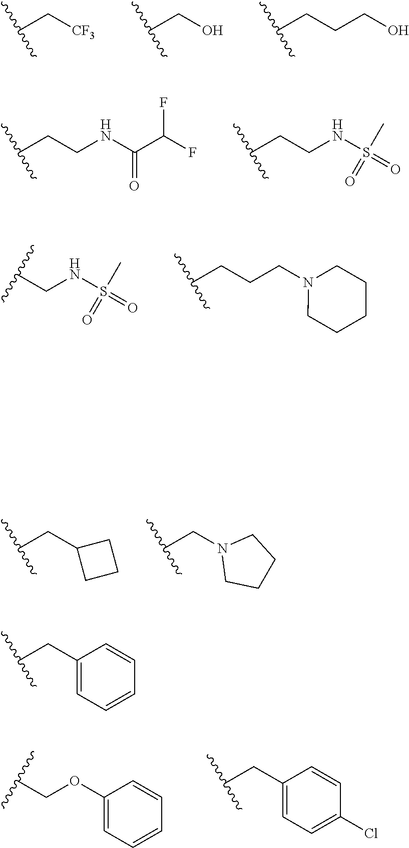

pharmaceutically acceptable salts, isomers and prodrugs thereof, wherein:
R² is selected from the group consisting of:

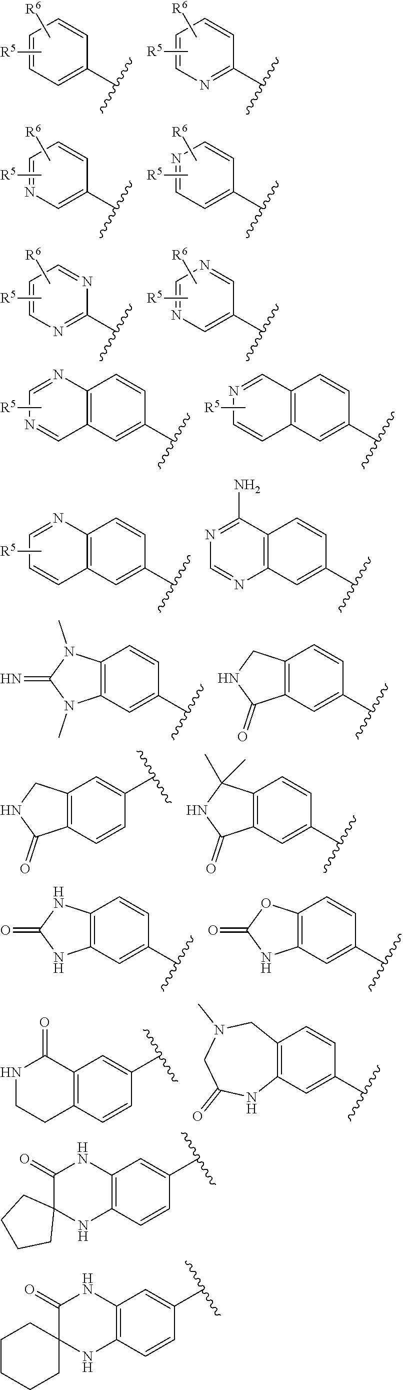

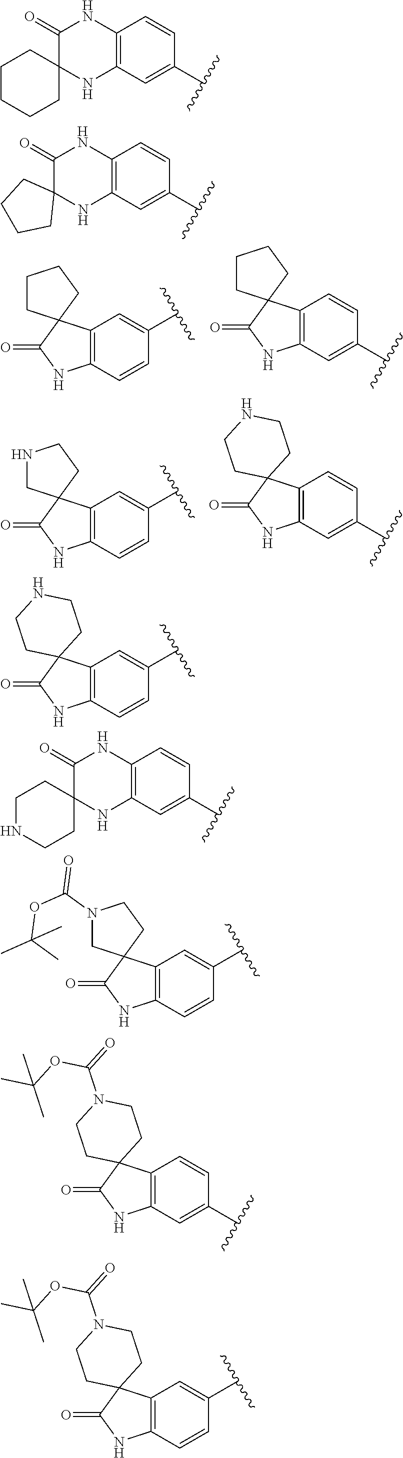

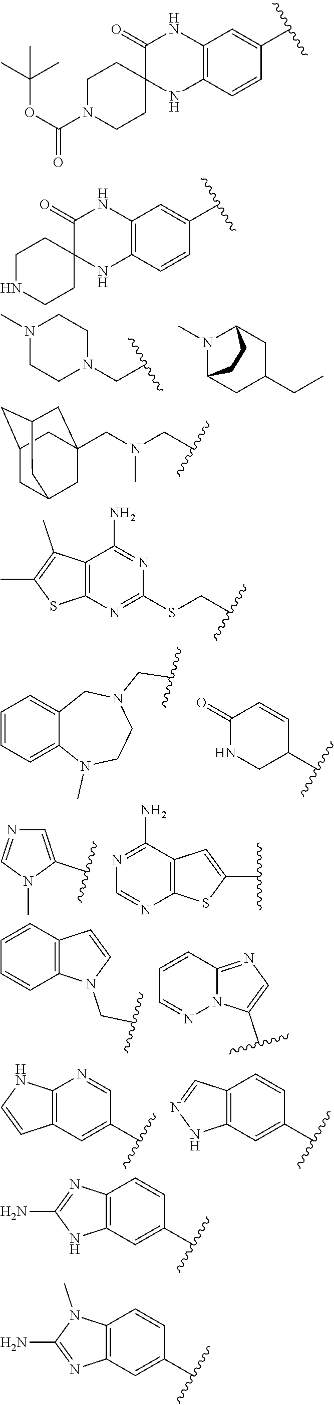

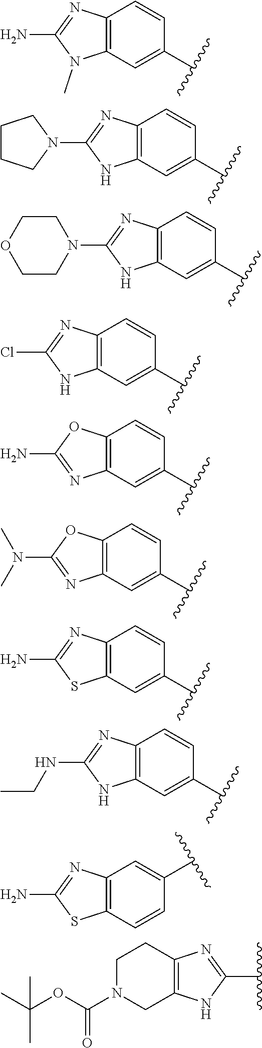

wherein at least one of R⁵ and R⁶ is selected from the group consisting of —OH, —NR′R″, —NHR⁷, —SO₂NMe₂, CH₂NH₂, —COR⁸ or is selected among the following substructures:

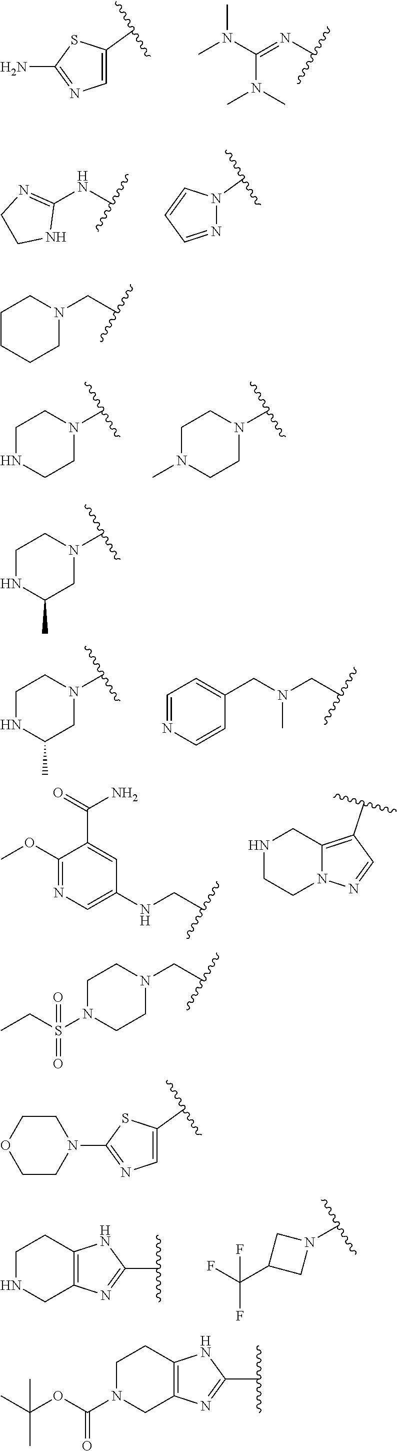

R⁷ is selected among the following substructures:

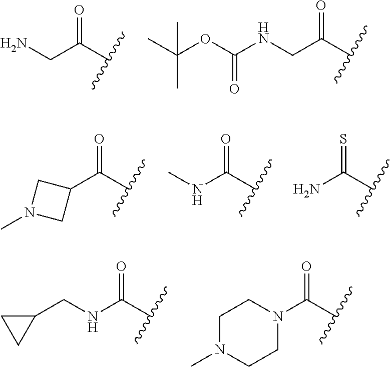

R⁸═—NR′R″ or selected among the following substructures:

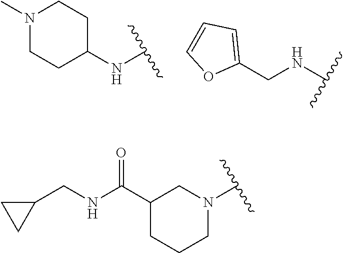

wherein R′ and R″ are independently —H or C₁-C₄ alkyl.

In this preferred embodiment, the R² substituents are polar groups, preferably H-donor groups.

Conversely, WO2020/212479 discloses that the R² substituent is preferably a relatively apolar group. The relatively apolar group is preferably a phenyl or phenyl substituted with alkyl, alkoxy, thioalkoxy or halogenated derivatives thereof, or halogen, most preferably substituted with halogen.

In another embodiment, when B═N, Z═CHR³ wherein R³ is H or C₁-C₄ alkyl, L is absent and each of X, X′, Y, Y′ are CH or one or two of X, X′, Y, Y′ are N, then R² is not selected from phenyl or pyridyl unsubstituted or substituted with one or more alkyl, alkoxy, thioalkoxy or halogenated derivatives thereof, or halogen, unsubstituted thiophenyl or furanyl.

In another embodiment, the following compounds are excluded:
2-(difluoromethyl)-5-(4-((5-phenyl-1H-tetrazol-1-yl)methyl)phenyl)-1,3,4-oxadiazole;
2-(difluoromethyl)-5-(6-((4-phenyl-1H-imidazol-1-yl)methyl)pyridin-3-yl)-1,3,4-oxadiazole;
2-(difluoromethyl)-5-(4-((4-phenyl-1H-1,2,3-triazol-1-yl)methyl)phenyl)-1,3,4-oxadiazole;
2-(4-((4-(4-chlorophenyl)-1H-1,2,3-triazol-1-yl)methyl)phenyl)-5-(difluoromethyl)-1,3,4-oxadiazole;
2-(difluoromethyl)-5-(4-((4-(4-(trifluoromethyl)phenyl)-1H-1,2,3-triazol-1-yl)methyl)phenyl)-1,3,4-oxadiazole;
2-(difluoromethyl)-5-(4-((4-(pyridin-4-yl)-1H-1,2,3-triazol-1-yl)methyl)phenyl)-1,3,4-oxadiazole;
2-(difluoromethyl)-5-(4-((4-(pyridin-3-yl)-1H-1,2,3-triazol-1-yl)methyl)phenyl)-1,3,4-oxadiazole;
2-(difluoromethyl)-5-(4-((4-(thiophen-2-yl)-1H-1,2,3-triazol-1-yl)methyl)phenyl)-1,3,4-oxadiazole;
2-(difluoromethyl)-5-(4-(1-(4-phenyl-1H-1,2,3-triazol-1-yl)ethyl)phenyl)-1,3,4-oxadiazole;
2-(difluoromethyl)-5-(4-((5-methyl-4-phenyl-1H-1,2,3-triazol-1-yl)methyl)phenyl)-1,3,4-oxadiazole;
2-(difluoromethyl)-5-(6-((4-phenyl-1H-1,2,3-triazol-1-yl) methyl) pyridin-3-yl)-1,3,4-oxadiazole;
2-(difluoromethyl)-5-(5-((4-phenyl-1H-1,2,3-triazol-1-yl) methyl) pyridin-2-yl)-1,3,4-oxadiazole;
2-(6-((4-(4-chlorophenyl)-1H-1,2,3-triazol-1-yl)methyl)pyridin-3-yl)-5-(difluoromethyl)-1,3,4-oxadiazole;
2-(6-((4-(2-chlorophenyl)-1H-1,2,3-triazol-1-yl)methyl)pyridin-3-yl)-5-(difluoromethyl)-1,3,4-oxadiazole;
2-(6-((4-(3-chlorophenyl)-1H-1,2,3-triazol-1-yl)methyl)pyridin-3-yl)-5-(difluoromethyl)-1,3,4-oxadiazole;
2-(6-((4-(3,4-dichlorophenyl)-1H-1,2,3-triazol-1-yl)methyl)pyridin-3-yl)-5-(difluoromethyl)-1,3,4-oxadiazole;
2-(6-((4-(3,5-dichlorophenyl)-1H-1,2,3-triazol-1-yl)methyl)pyridin-3-yl)-5-(difluoromethyl)-1,3,4-oxadiazole;
2-(difluoromethyl)-5-(6-((4-(2-fluorophenyl)-1H-1, 2, 3-triazol-1-yl)methyl)pyridin-3-yl)-1,3,4-oxadiazole;
2-(difluoromethyl)-5-(6-((4-(2,6-difluorophenyl)-1H-1,2,3-triazol-1-yl)methyl)pyridin-3-yl)-1,3,4-oxadiazole;
2-(6-((4-(3-chlorophenyl)-1H-1,2,3-triazol-1-yl)methyl)pyridin-3-yl)-5-(difluoromethyl)-1,34-oxadiazole; and
2-(difluoromethyl)-5-(6-((4-(3,5-difluorophenyl)-1H-1,2,3-triazol-1-yl)methyl)pyridin-3-yl)-1,3,4-oxadiazole.

Another class of preferred compounds comprises compounds of formula (I) and pharmaceutically acceptable salts, isomers and prodrugs thereof, wherein:

X and X′ are independently selected from CH, N or CF;
Y and Y′ are independently selected from CH, N or CF;

### A=C, N, S;

### B═C, N;

### D=C, N;

### E=C, N, O;

### M=C;

Z═CH₂, CHR³;
R³=Me, or can be selected among the following substructures:

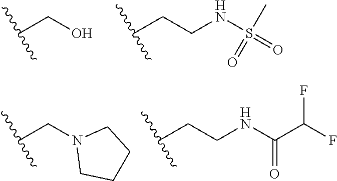

L is absent;
R² is selected from the group consisting of:

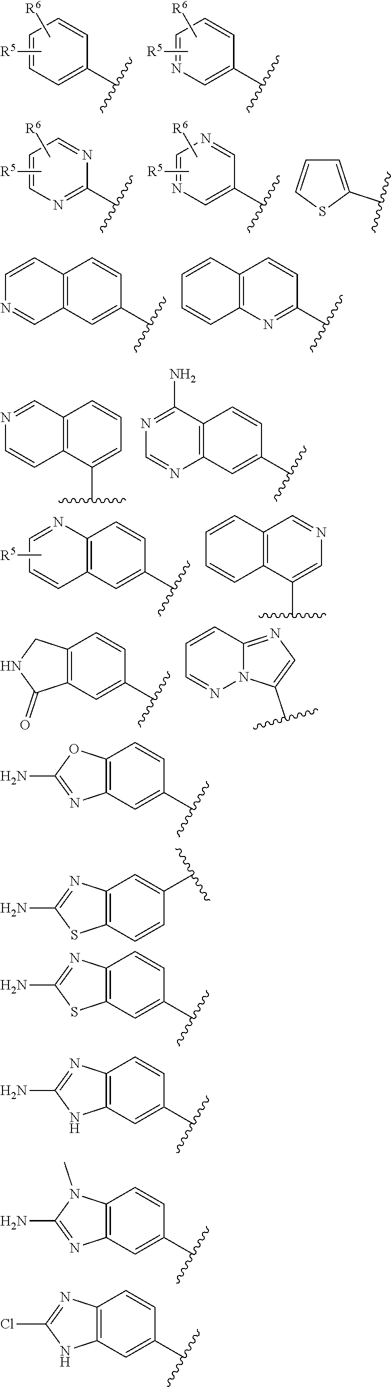

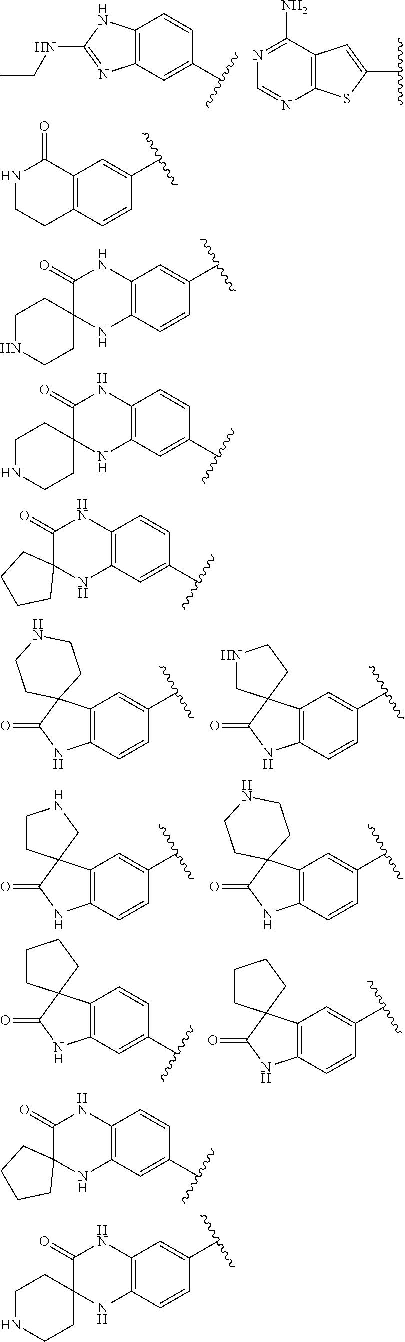

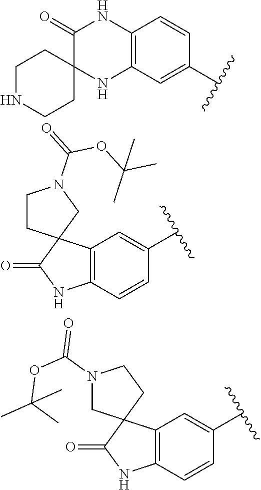

R⁵ and R⁶ are independently selected from the group comprising: —OH, —OMe, —Br, NH₂, —NHR⁷, —COR⁸, —COCH₃, —CH₃, —CH₂NH₂, or can be selected among the following substructures:

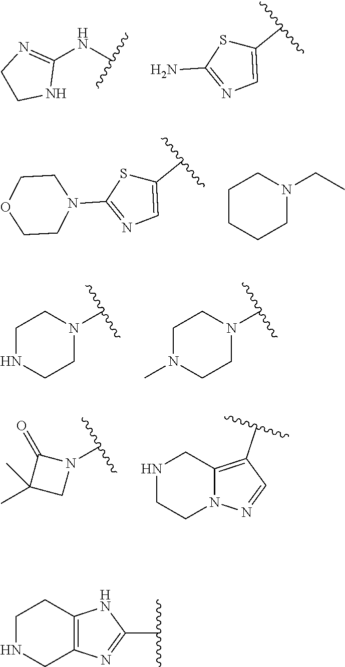

R⁷=Me, Et, or can be selected among the following substructures:

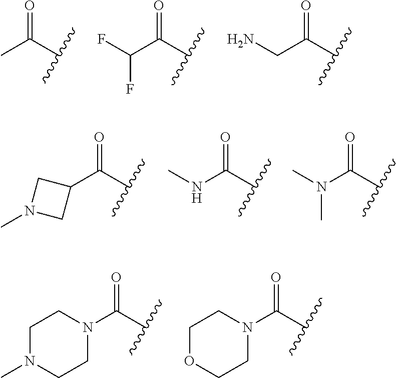

R⁸═—NH₂,—NHEt, —NMe₂, or can be selected among the following substructures:

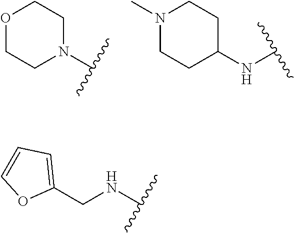

The following compounds of formula (I) are particularly preferred: compounds from (1) to (67), (69), (71), (72), (252), (264), (265), (269), (270), (273), (274), (276), (292), (293), (306), (307), (339), (340), from (345) to (348), (350), (351), (356), (359), (362), (376), (382), from (477) to (482).

Compounds of the present invention may contain one or more chiral centres (asymmetric carbon atoms), therefore they may exist in enantiomeric and/or diastereoisomeric forms.

All possible optical isomers, alone or in a mixture with each other, fall within the scope of the present invention.

Compounds according to the invention may be used alone or in combination with other drugs such as proteasome inhibitors, immunochemical inhibitors, steroids, bromodomain inhibitors and other epigenetic drugs, traditional chemotherapeutic agents, such as, for example, but not limited to, cisplatin, taxol, proteasome inhibitors, such as, for example, but not limited to, bortezomib, kinase inhibitors, such as, for example, but not limited to, JAK family, CTLA4, PD1 or PDL1 checkpoints inhibitors, such as nivolumab, pemprolizumab, pidilizumab or BMS-936559 (anti-PD1), atezolizumab or avelumab (anti-PDL1), ipilimumab or tremelimumab (anti-CTLA4). The compounds of the invention alone or in combination are preferably useful for the treatment of HDAC6-mediated diseases.

The compounds of the invention alone or in combination are preferably useful for the treatment of peripheral neuropathies, both genetically originated, such as, for example, but not limited to, Charcot-Marie-Tooth disease, medication induced (chemotherapy or antibiotics, such as metronidazole and fluoroquinolone classes) and due to systemic diseases, such as diabetes or leprosy or in general for the treatment of peripheral neuropathies correlated to severe axonal transport deficit. The compounds of invention can also be useful for treatment of chemotherapy-related cognitive impairment (CRCI). The compounds of the invention alone or in combination are preferably useful for the treatment of graft rejection, GVHD, myositis, diseases associated with abnormal lymphocyte functions, multiple myeloma, non-Hodgkin lymphoma, peripheral neuropathy, autoimmune diseases, inflammatory diseases, cancer and neurodegenerative diseases, ocular diseases (e.g. uveitis).

Therefore, the present invention also provides pharmaceutical compositions comprising a therapeutically effective amount of compounds of formula (I) or pharmaceutically acceptable salts, isomers and pharmacologically acceptable prodrugs thereof, together with at least one pharmaceutically acceptable excipient. Such compositions can be liquid, suitable for enteral or parenteral administration, or solid, for example, in the form of capsules, tablets, pills, powders or granules for oral administration, or in forms suitable for cutaneous administration such as creams or ointments, or for inhalation delivery.

The pharmaceutical compositions of the present invention can be prepared by using known methods.

General Synthetic Pathway

The compounds described in the present invention can be prepared by using methods known to those skilled in the art.

All starting materials, reagents, acids, bases, solvents and catalysts used in the synthesis of the described compounds are commercially available.

Reaction progression was monitored by TLC, HPLC, UPLC or HPLC-MS analysis. 2-(difluoromethyl)-1,3,4-oxadiazole moiety was synthesized in most of the cases treating the corresponding hydrazide with an excess of difluoroacetic anhydride (see scheme 1). This reagent has a double function of acylating and dehydrating agent. (Lee, Jaekwang; Han, Younghue; Kim, Yuntae; Min, Jaeki; Bae, Miseon; Kim, Dohoon; Jin, Seokmin; Kyung, Jangbeen; 2017; “1,3,4-Oxadiazole sulfonamide derivatives as histone deacetylase 6 inhibitors and their pharmaceutical composition and preparation”; WO2017018805). In some cases, 2-(difluoromethyl)-1,3,4-oxadiazole moiety was prepared starting from the corresponding tetrazole, which was converted into 2-(difluoromethyl)-1,3,4-oxadiazole in presence of difluoroacetic anhydride (Vereshchagin et al Rus. J. Org. Chem. 2007, 43(11), 1710-1714).

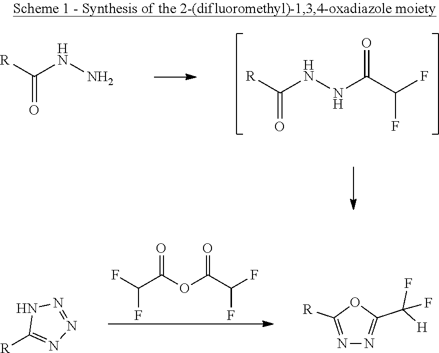

Appropriate common intermediates (different according to the central heterocycle scaffold) were synthesized, in order to prepare various compounds bearing different “cap terms” by assembling the central heterocycles. In a few cases the 2-(difluoromethyl)-1,3,4-oxadiazole moiety was synthesized in the last step.

As regards 1,2,3-triazole containing compounds, the common intermediate was a 2-(4-(azidomethyl)aryl)-5-(difluoromethyl)-1,3,4-oxadiazole, which underwent a Cu(I)-catalyzed azide/alkyne cycloaddition with an appropriate derivatized alkyne, in water/DMSO, using copper(II) sulfate and (+)-sodium L-ascorbate as the catalytic system (see scheme 3) (in plate: T. Suzuki et al. J. Med. Chem. 2012, 55(22), 9562-9575; batch: T. U. Connell et al. J. Label Compd. Radiopharm. 2014, 57, 262-269). These intermediate azidomethyl-derivatives were prepared from the corresponding methyl 4-methylbenzoate, which was first converted into the difluoromethyl-1,3,4-oxadiazole via hydrazide as described above, then brominated by treatment with N-bromosuccinimide (NBS) and azobisisobutyronitrile (AIBN) or benzoyl peroxide as a catalyst. The azido moiety was introduced by nucleophilic substitution treating the obtained bromide with sodium azide (scheme 2). For fluorinated and chlorinated aryl-derivatives, the construction of the 2-(difluoromethyl)-1,3,4-oxadiazole moiety was performed after the introduction of the azido group (scheme 2).

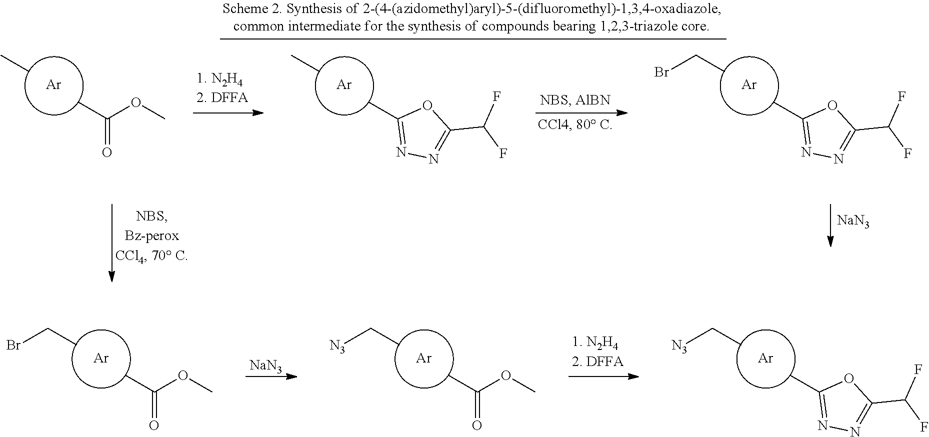

In the case of pyridazine derivatives, the 2-(difluoromethyl)-1,3,4-oxadiazole moiety was synthesized in the last step.

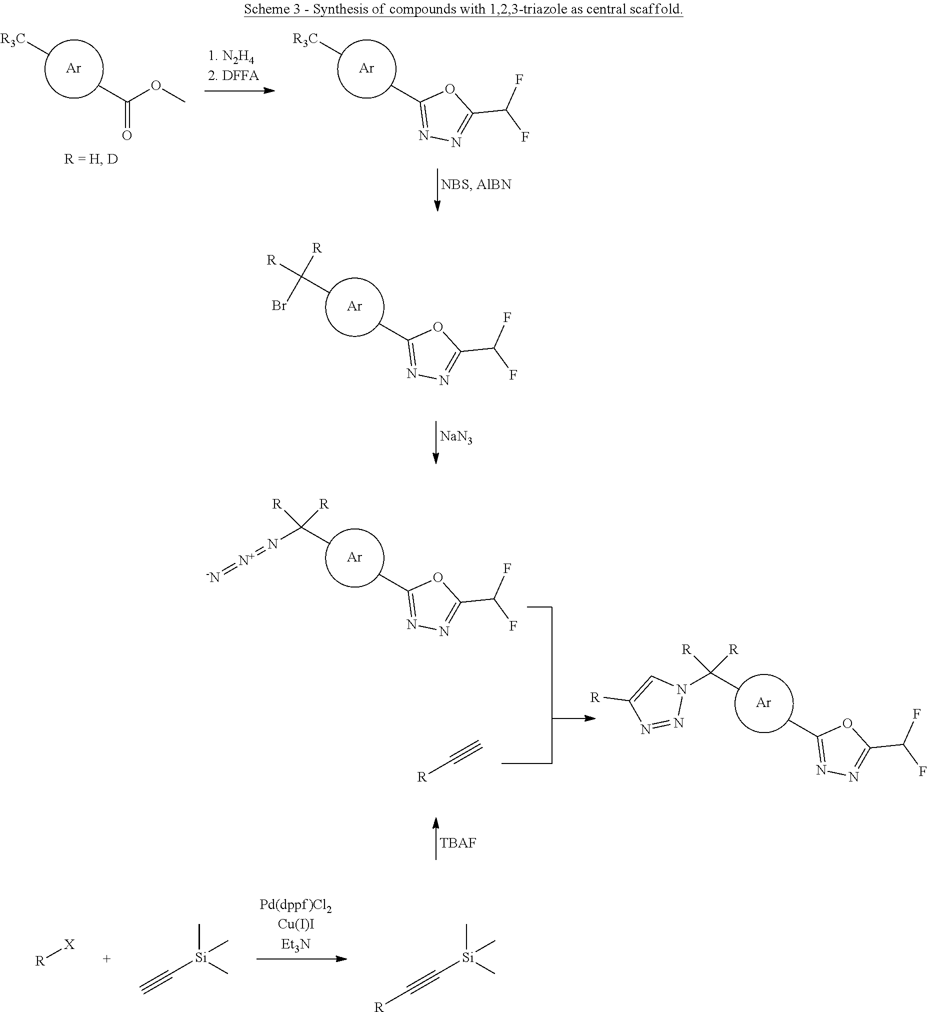

Most of the alkynes used in the synthesis of these 1,2,3-triazole containing analogues were commercially available. Non-commercial building blocks were synthesized via Sonogashira coupling, reacting the appropriate halogen-derivative with ethynyl(trimethyl)silane in the presence of triethylamine, using [1,1′-Bis(diphenylphosphino)ferrocene]dichloropalladium(II) (Pd(dppf)Cl₂) and copper(I) iodide as catalysts, and subsequent removal of the silyl protecting group with tetrabutylammonium fluoride (TBAF) (Scheme 3). (A. G. Sams et al Bioorg. Med. Chem. Lett. 2011, 21(11), 3407-3410).

When Z═CHR the same synthetic route was followed to form the 1,2,3-triazole core scaffold. The synthesis of the proper azides followed diverse strategies, depending on the R group (scheme 4). In some cases, the azide was installed by nucleophilic substitution of a bromide or of an activated hydroxy group (mesylate), treated with sodium azide. In this last case, the alcohol precursor was obtained either from an aldehyde, which underwent Grignard or Barbier reactions, or by reduction of a ketone with sodium borohydride. For =ethylmethanesulfonamide, both ketone and nitrile were reduced by employing catalytic amounts of nickel(II) chloride with excess sodium borohydride, trapping the primary amine with Boc₂O (S. Caddick et al. Tetrahedron 2003, 59, 5417-5423). The proper ketone was either commercially available or could be accessed applying known methods; for example, by reacting a suitable carboxylic acid with (4-(methoxycarbonyl)phenyl)boronic acid (L. J. Gooßen et al. Eur. J. Org. Chem. 2002, 3254-3267). When R was —CH₂OH, the corresponding azide was obtained by opening the epoxide ring of a methyl 4-(oxiran-2-yl)benzoate derivative with sodium azide, directly. Finally, when R was —CH₂CF₃, azide was prepared treating methyl 4-vinylbenzoate with Togni's reagent, TMS-N₃ and a catalytic amount of [Cu(CH₃CN)₄]PF₆. (Wang, F., Qi, X., Liang, Z., Chen, P. and Liu, G. (2014), Copper-Catalyzed Intermolecular Trifluoromethylazidation of Alkenes: Convenient Access to CF₃-Containing Alkyl Azides. Angew. Chem. Int. Ed., 53: 1881-1886).

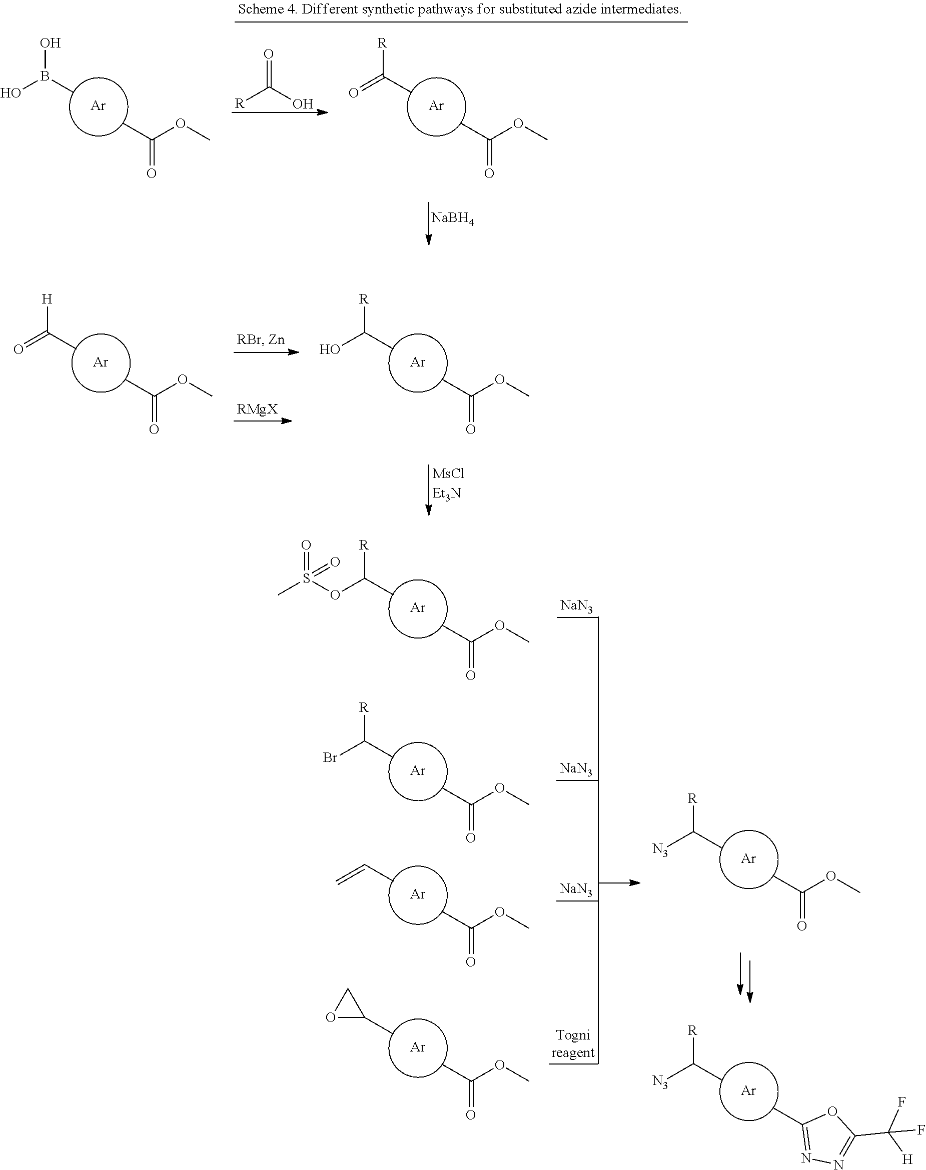

Compounds bearing tetrazole, imidazole and pyrazole as central scaffolds were synthesized by nucleophilic substitution, reacting the common intermediate 2-(4-(bromomethyl)aryl)-5-(difluoromethyl)-1,3,4-oxadiazole with appropriate substituted tetrazoles, pyrazoles or imidazoles at room temperature overnight, in DMF using potassium carbonate as base (see scheme 5). The common intermediate methylbromide-derivative was synthesized as described for 1,2,3-triazole core bearing compounds (scheme 2). In a few cases, the 2-(difluoromethyl)-1,3,4-oxadiazole moiety was synthesized in the last step. In other few cases the bromine-intermediate was reacted with iodo-pyrazole and the R group was inserted in the last step via Stille or Suzuki reaction. Other non commercially available substituted imidazoles or pyrazoles were prepared coupling N-THP-protected imidoyl- or pyrazolyl-pinacol boronate with a suitable aryl halide under Suzuki conditions. THP protection was afterwards removed in acidic conditions, prior to the alkylation step. In a few cases, imidazole ring was formed reacting the suitable bromomethyl ketone with formamide (Cong et al. J. Chem. Res. 2014, 38(4), 208-210).

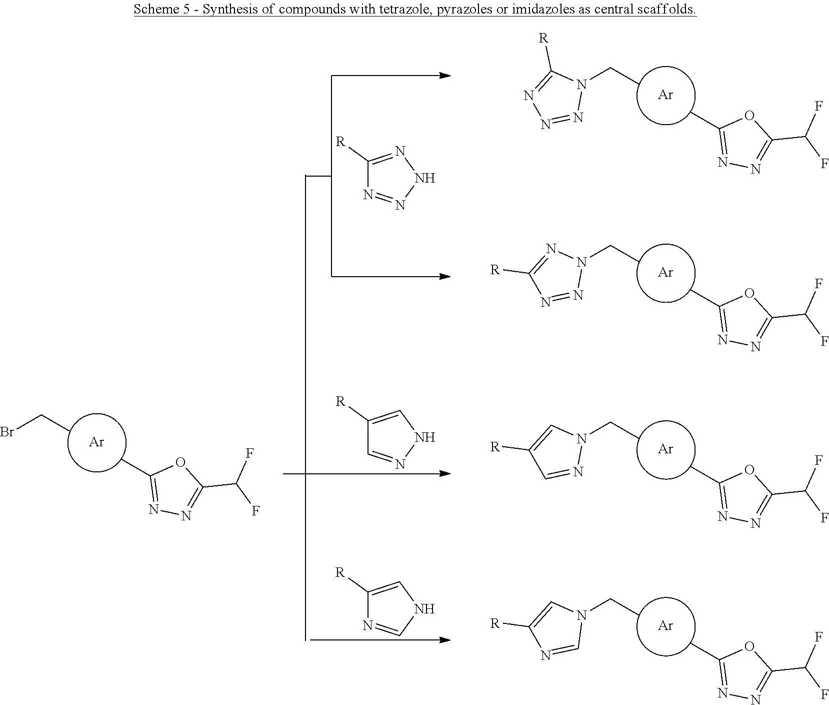

Most of the substituted tetrazoles used were commercially available. Non-commercial building blocks were synthesized from the corresponding carbonitrile by reaction with an excess of sodium azide in the presence of ammonium chloride.

Compounds bearing isoxazole as a central scaffold were obtained via Sonogashira reaction, by reacting 2-(difluoromethyl)-5-(4-iodoaryl)-1,3,4-oxadiazole with ethynyl(trimethyl)silane and triethylamine, in the presence of Cul and [1,1′-Bis(diphenylphosphino)ferrocene]dichloropalladium(II) (Pd(dppf)Cl₂) as catalysts. The trimethylsilyl-protection was removed one pot by treatment with tetrabutylammonium fluoride (scheme 6). The obtained product underwent Glazer coupling with an appropriate alkyne in the presence of copper(II) acetate (B. Nammalwar et al WO2017083434 2017; Ding, Shi et al Bioorg. Med. Chem. Lett. 2018, 28(2), 94-102), providing an open intermediate, which was cyclized by treatment with hydroxylamine hydrochloride and triethylamine at 110° C. (L. Wang et al Org. Lett. 2012, 14(9), 2418-2421). In the case of compounds bearing oxazole as a core scaffold 2-(difluoromethyl)-5-(4-iodoaryl)-1,3,4-oxadiazole underwent Sonogashira reaction with the corresponding propynyl amide in presence of bis(triphenylphosphine)palladium(II) dichloride and copper iodide. Oxazole ring was cyclized in presence of diazabibycloundecene (DBU).

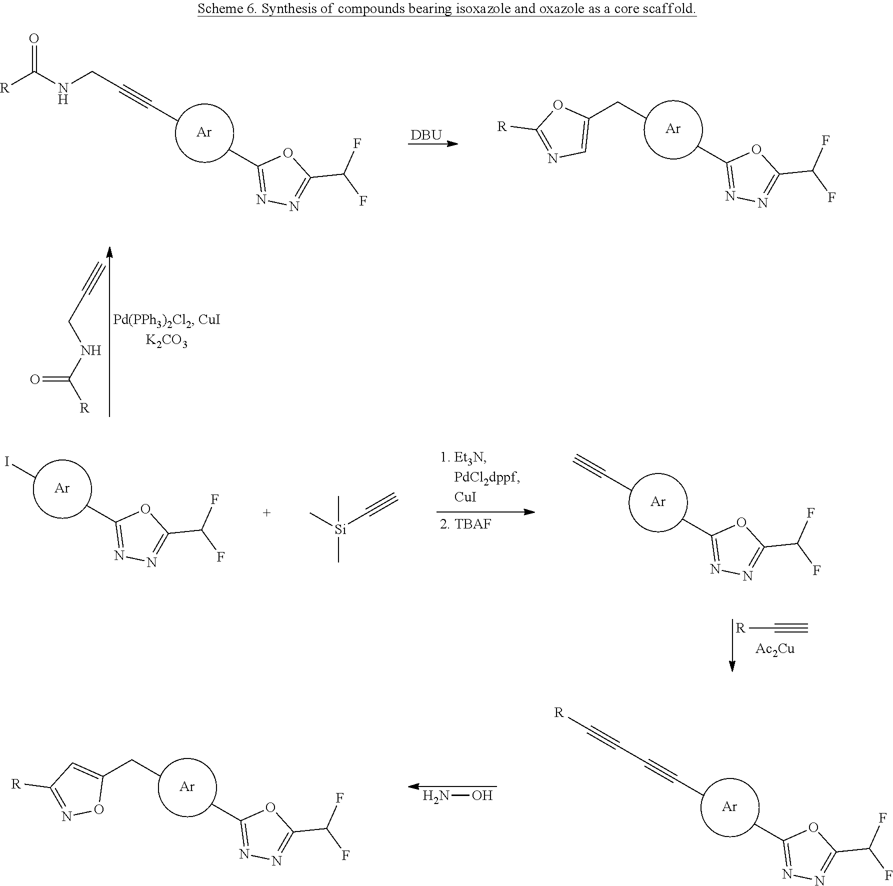

Compounds with 1,2,4-oxadiazole core were synthesized reacting a carboxylic acid with the properly substituted N′-hydroxybenzimidamide, in presence of EDC and HOBT. These two moieties can be installed either in benzylic position on the ZBG side or on the cap-term, depending on the desired structural isomer (scheme 7). The N′-hydroxybenzimidamide was previously obtained treating the corresponding nitrile with hydroxylamine hydrochloride in presence of sodium hydrogencarbonate (S. D. Diwakar et al J. Het. Chem. 2011, 48(4), 882-887; F. Yokokawa et al J. Med. Chem. 2016, 59(8), 3935-3952). In most of the cases, the 2-(difluoromethyl)-1,3,4-oxadiazole moiety was synthesized in the last step of the synthesis, starting from the corresponding methyl ester, or from the corresponding nitrile. Nitrile was treated with sodium azide to generate tetrazole, which was converted to 2-(difluoromethyl)-1,3,4-oxadiazole in presence of difluoroacetic anhydride. When Z═CF₂, the 2-(difluoromethyl)-1,3,4-oxadiazole moiety was formed in the first step on methyl 4-iodobenzoate. The resulting intermediate was treated with ethyl 2-bromo-2,2-difluoroacetate in presence of copper powder to obtain an ethyl ester (M.-T. Hsieh et al Adv. Synth. Cat. 2018, 360(8), 1605-1610), which was hydrolyzed to a carboxylate common intermediate with LiOH.

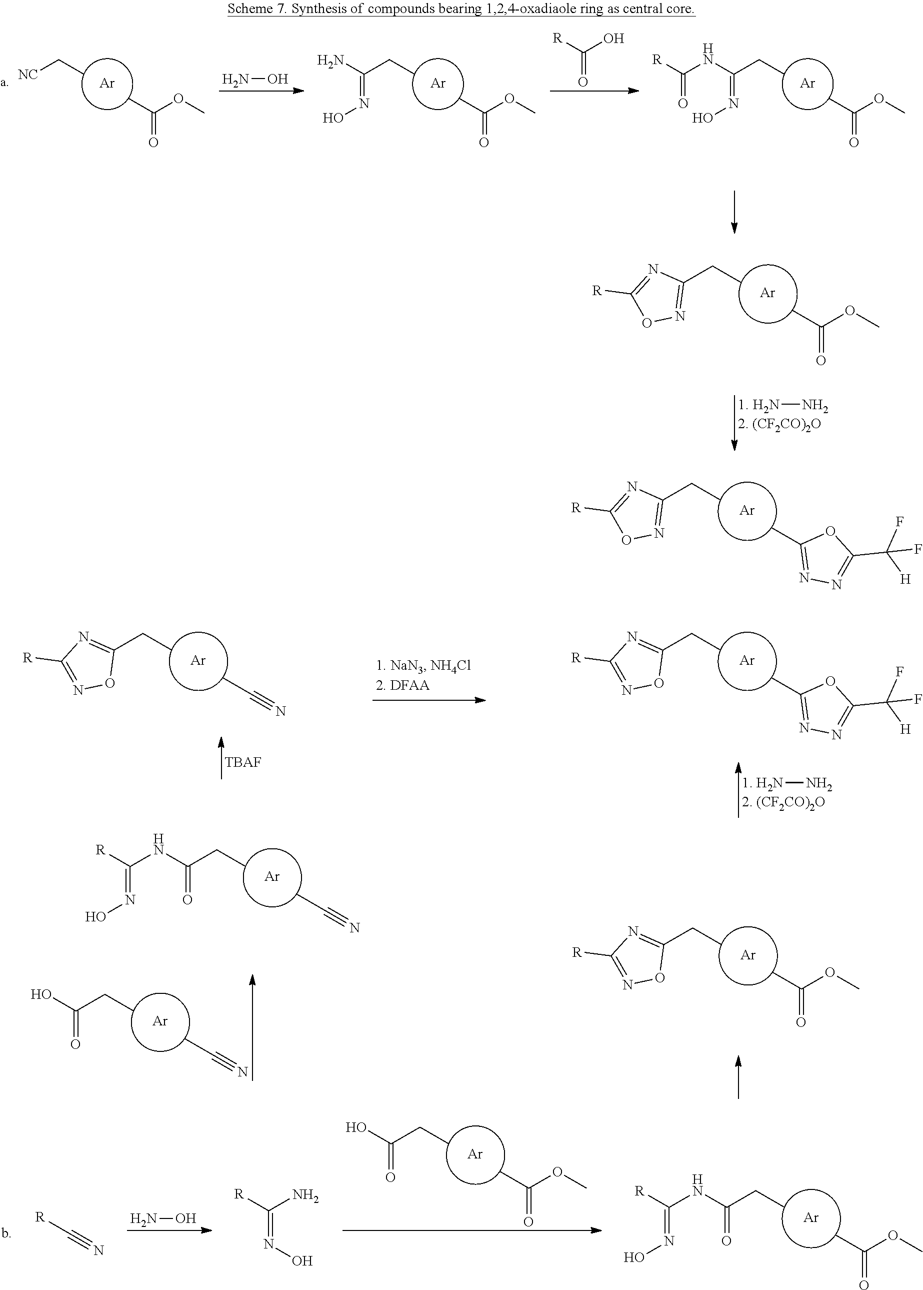

The obtained methyl ester intermediates in fact were treated with hydrazine in order to obtain the corresponding hydrazides, which undergoes acylation and cyclization in the presence of difluoroacetic anhydride (scheme 7).

Compounds bearing 1,3,4-oxadiazole and 1,3,4-thiadiazole core were synthesized coupling 2-(4-(methoxycarbonyl)phenyl)acetic acid, or the appropriate aryl analogue, with a substituted benzohydrazide and treating the linear intermediate with a dehydrating agent in order to obtain the cyclic desired product. 1,3,4-oxadiazoles were prepared using Burgess' reagent as cyclizing agent (Lv. Fengping et al Bioorg. Med. Chem. Lett. 2016, 26(15), 3714-3718) and 1,3,4-thiadiazoles were prepared using Lawesson's reagent (Scheme 8) (B. Sybo et al J. Mater. Chem. 2007, 17, 3406-3411; J. Slawinski et al Eur J. Med. Chem. 2014, 82, 47-55). The obtained methyl esters were converted into the corresponding 2-(difluoromethyl)-1,3,4-oxadiazole, by treatment with hydrazine first and then with difluoroacetic anhydride.

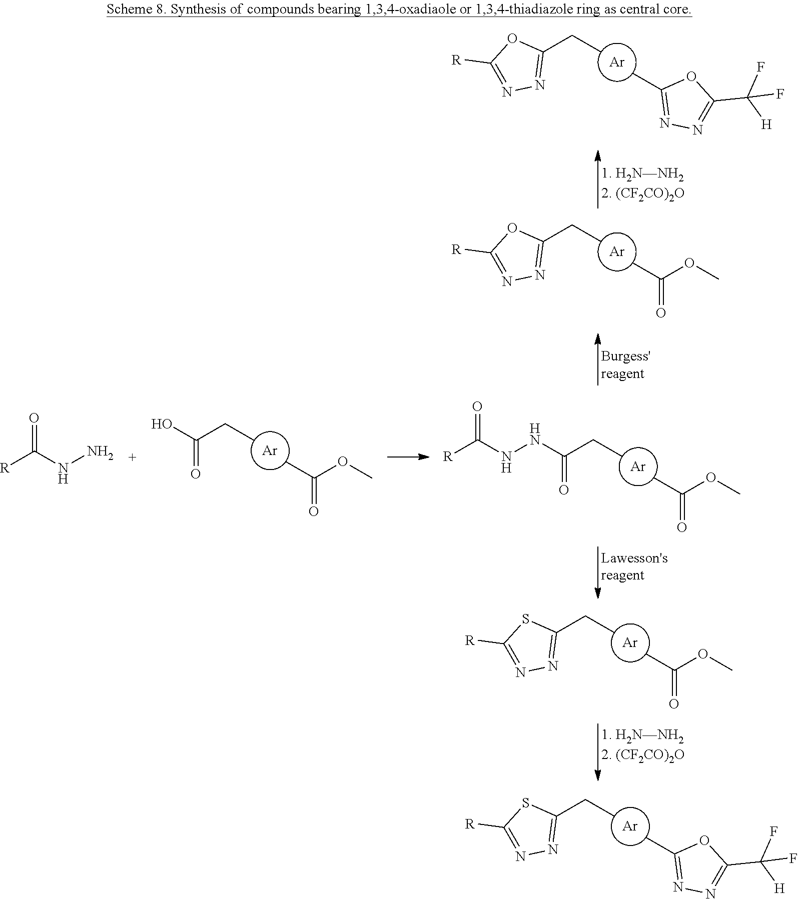

The triazole-thiol core compounds were obtained by reaction of 1,2,4-triazole-thiols, optionally substituted, with 2-(difluoromethyl)-5-(4-iodophenyl)-1,3,4-oxadiazole or 2-(difluoromethyl)-5-(3,4,5-trifluorophenyl)-1,3,4-oxadiazole, in the presence of potassium carbonate in DMF under heating overnight. The reaction with 2-(difluoromethyl)-5-(4-iodophenyl)-1,3,4-oxadiazole was catalyzed with copper iodide and L-proline (Scheme 9) and was heated at 80° C. (Liang-Feng et al., Tetrahedron (2011), 67, 2878-2881). On the other hand, the reaction with 2-(difluoromethyl)-5-(3,4,5-trifluorophenyl)-1,3,4-oxadiazole proceeds even under mild conditions (70° C.) and without catalysis (Scheme 9) (Dudutiene et al., Bioorg. Med. Chem. (2013), 21(7), 2093-2106; WO03/062225).

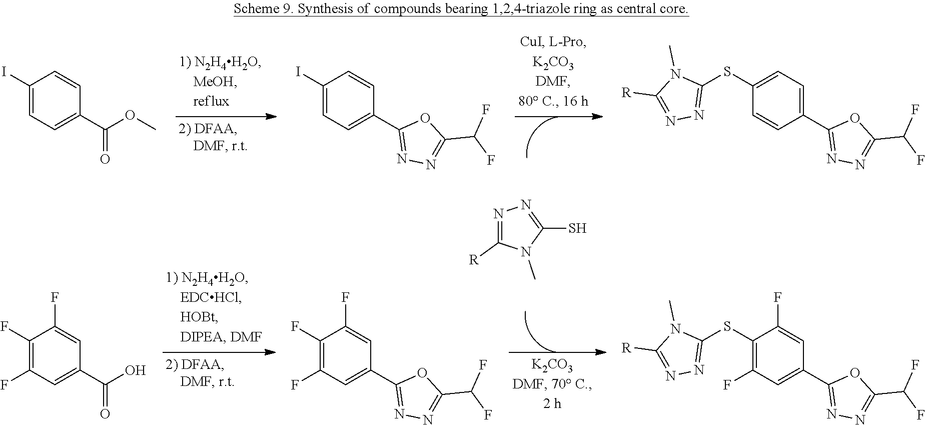

2-(difluoromethyl)-1,3,4-oxadiazole moiety was prepared, as already described, from the corresponding hydrazide. 4-iodobenzohydrazide was synthesized starting from methyl 4-iodobenzoate in the presence of hydrazine monohydrate, in methanol under reflux. 3,4,5-trifluorobenzohydrazide was obtained by treating 3,4,5-trifluorobenzoic acid with EDC, HOBt and DIPEA in the presence of hydrazine monohydrate.

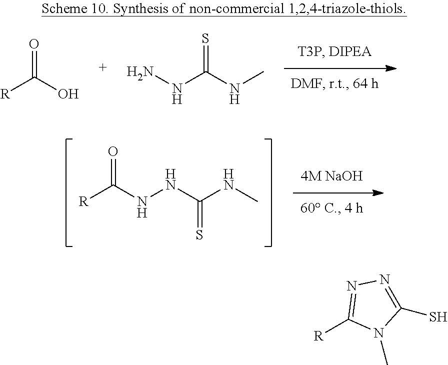

Many of the starting 1,2,4-triazole-thiols are commercially available. In some cases, they have been synthesized according to the route shown in Scheme 10. The open intermediate was prepared from carboxylic acid by activation with T3P and condensation with N-methyl hydrazine carbothioamide in the presence of DIPEA in DMF (US2007/0232808). Cyclization of the open intermediate was achieved by addition of aqueous NaOH to the reaction mixture.

Compounds bearing a 1,2,3-triazole core scaffold having B═C and M=N were prepared by Copper-Catalyzed Azide-Alkyne Cycloaddition, in the already described conditions. The alkynyl intermediate was prepared from the common intermediate 2-(4-(bromomethyl)aryl)-5-(difluoromethyl)-1,3,4-oxadiazole by Grignard reaction, in presence of a catalytic amount of Pd(dppf)Cl₂·DCM complex. In some cases the 2-(difluoromethyl)-1,3,4-oxadiazole moiety was introduced as the last step, via hydrazide. Azides, when not commercially available, were prepared either from the corresponding aryl boronic acids, treated with tetrabutylammonium fluoride and trimethylsilyl azide in presence of copper chloride as a catalyst (Yu et al Chem. Eur. J. 2010 16(27), 7969-7972), or from a suitable aryl iodide, by reaction with sodium azide in the presence of sodium ascorbate, copper iodide and N,N′-dimethylethane-1,2-diamine (Wang et al. Tetrahedron Lett. 2011, 52, 3295-3297).

The following examples are intended to further illustrate the invention but not limiting it.

### Example 1. Synthesis of 2-(6-(bromomethyl)pyridin-3-yl)-5-(difluoromethyl)-1,3,4-oxadiazole (Intermediate A)

Step A

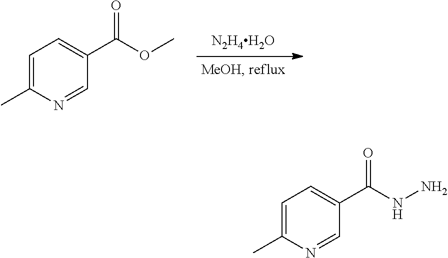

Methyl 6-nicotinate (4 g, 1 equiv.) was dissolved in MeOH (25 mL), then hydrazine monohydrate was added (5 equiv.) under stirring. Mixture was refluxed over 3 h. Full conversion of methyl ester to hydrazide was observed by LC-MS (and TLC). The reaction mixture was concentrated and dried under vacuum. The white solid obtained (3.93 g) was used for the subsequent step without further purification.

Step B

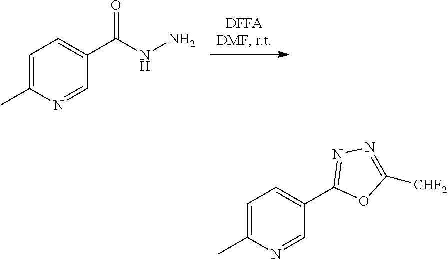

Hydrazide obtained in step A (3.93 g, 1 equiv.) was dissolved in dry DMF (30 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. Full conversion was observed by LC-MS.

Sat. aq. NaHCO₃ was added to the reaction mixture to quench difluoroacetic anhydride excess. Then water was added, and the product was extracted with ethyl acetate (3×). Organic layers were collected together, washed with sat. aq. NaHCO₃ and brine, dried over Na₂SO₄ and evaporated to dryness under reduced pressure. The crude yellow oil obtained (5.43 g) was used in the next step without further purification.

Step C

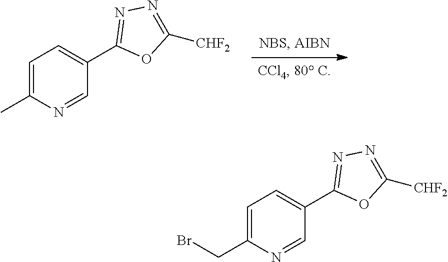

2-(difluoromethyl)-5-(6-methylpyridin-3-yl)-1,3,4-oxadiazole (1 g, 4.7 mmol, 1 equiv.) was dissolved in 20 mL degassed carbon tetrachloride. N-Bromosuccinimide (NBS, 1.2 equiv.) and azobisisobutyronitrile (AIBN, 0.04 equiv.) were added to the reaction mixture, which was stirred at 80° C. overnight.

Solution was diluted with water, extracted with DCM, dried over MgSO₄ and concentrated under reduced pressure to dryness.

Purification by flash column chromatography (hexane/EtOAc 9:1) afforded the desired product as a violet solid (623 mg, 45% yield).

The following compounds were prepared according to the same procedure:

| Compd. | Structure |
|---|---|
| Intermediate B | 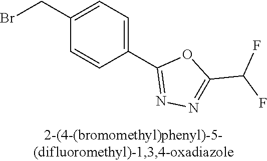 [SMILES: FC(F)c1nnc(-c2ccc(CBr)cc2)o1] |
| Intermediate C | 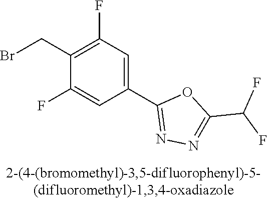 [SMILES: Fc1cc(-c2nnc(C(F)F)o2)cc(F)c1CBr] |
| Intermediate D | 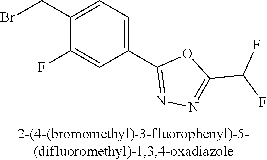 [SMILES: Fc1cc(-c2nnc(C(F)F)o2)ccc1CBr] |
| Intermediate E | 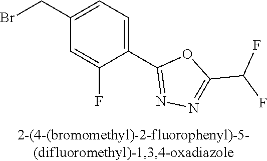 [SMILES: Fc1cc(CBr)ccc1-c1nnc(C(F)F)o1] |
| Intermediate Z | 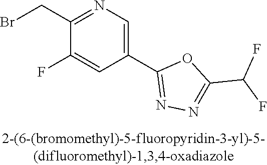 [SMILES: Fc1cc(-c2nnc(C(F)F)o2)cnc1CBr] |

### Example 2. Synthesis of 2-(6-(azidomethyl)pyridin-3-yl)-5-(difluoromethyl)-1,3,4-oxadiazole (Intermediate F)

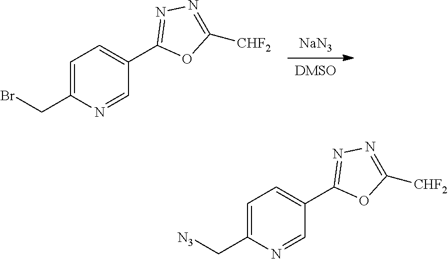

A solution of 2-(6-(bromomethyl)pyridin-3-yl)-5-(difluoromethyl)-1,3,4-oxadiazole (Intermediate F, 82 mg, 0.285 mmol, 1 equiv.) and sodium azide (1 equiv.) in 0.5 mL DMSO was stirred at r.t. for 1 h. Conversion was confirmed by LC-MS (98%). The reaction mixture was filtered and used directly for the subsequent step.

The following compound was prepared according to the same procedure:

| Compd. | Structure |
|---|---|
| Intermediate G | 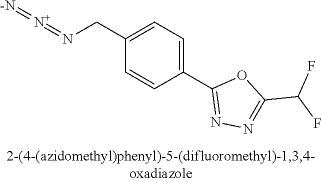 |

### Example 3. Synthesis of 2-(4-(azidomethyl)-2,3-difluorophenyl)-5-(difluoromethyl)-1,3,4-oxadiazole (Intermediate H)

Step A

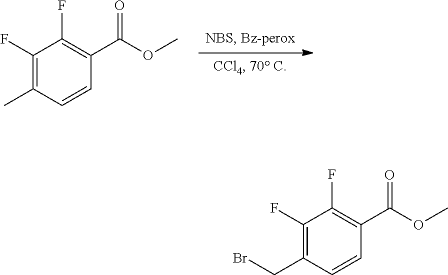

Methyl 2,3-difluoro-4-methylbenzoate (2 g, 10.7 mmol, 1 equiv.) and N-Bromosuccinimide (NBS, 1.05 equiv.) were dissolved in 40 mL degassed carbon tetrachloride. Then benzoyl peroxide (0.05 equiv.) was added to the reaction mixture, which was stirred at 70° C. overnight. The mixture was let to reach r.t., then diluted with DCM and washed successively with sat. aq. NaHCO₃, water and brine. The organic layer was separated, dried over MgSO₄, filtered and concentrated under reduced pressure affording a colorless oil which was purified by flash column chromatography (hexane/EtOAc 95:5) affording the product as a white solid (1.72 g, 6.49 mmol, 60.4% yield).

Step B

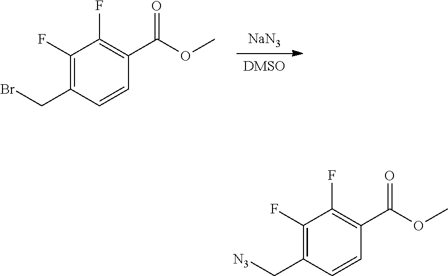

A solution of methyl 4-(bromomethyl)-2,3-difluorobenzoate (1.72 g, 6.49 mmol, 1 equiv.) and sodium azide (1.4 equiv.) in 20 mL DMSO was stirred at r.t. overnight. The reaction was quenched with water and extracted with ethyl acetate. The organic layer was washed with brine, dried over MgSO₄, filtered and concentrated under reduced pressure to afford the product as a yellow oil (1.41 g, 6.21 mmol, 95% yield) which was used in the next step without further purification.

Step C

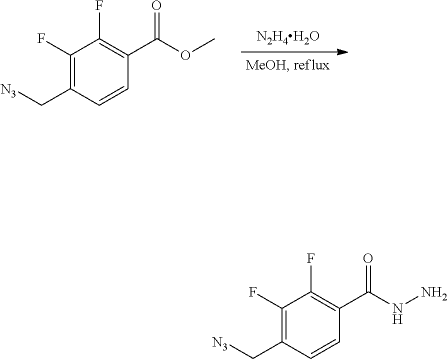 [SMILES: COC(=O)c1ccc(CN=[N+]=[N-])c(F)c1F ; "N2H4·H2O" ; "MeOH, reflux" ; [N-]=[N+]=NCc1ccc(C(=O)NN)c(F)c1F]

Methyl 4-(azidomethyl)-2,3-difluorobenzoate (1.38 g, 1 equiv.) was dissolved in MeOH (20 mL), then hydrazine monohydrate was added (4 equiv.) under stirring. Mixture was stirred at 65° C. overnight. Full conversion of methyl ester to hydrazide was observed by LC-MS (and TLC). The reaction mixture was concentrated and the residue was triturated in water. The white solid obtained was filtered, washed with water and dried under vacuum (1.17 g, 84% yield). The product was used for the subsequent step without further purification.

Step D

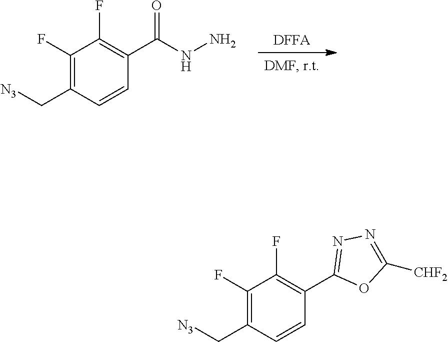 [SMILES: [N-]=[N+]=NCc1ccc(C(=O)NN)c(F)c1F ; "DFFA" ; "DMF, r.t." ; [N-]=[N+]=NCc1ccc(-c2nnc(C(F)F)o2)c(F)c1F]

Hydrazide obtained in step C (584 mg, 1 equiv.) was dissolved in dry DMF (30 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. Full conversion was observed by LC-MS.

Sat. aq. NaHCO₃ was added to the reaction mixture to quench difluoroacetic anhydride excess. Then water was added, and the product was extracted with ethyl acetate (3×). Organic layers were collected together, washed with sat. aq. NaHCO₃ and brine, dried over Na₂SO₄ and evaporated to dryness under reduced pressure. Sufficiently pure product was obtained as a yellow oil which solidified (701 mg, 95% yield), and was used in the next step without further purification.

The following building blocks were prepared following the same procedure, starting from the corresponding bromide (step B):

| Compd. | Structure |
|---|---|
| Intermediate I | 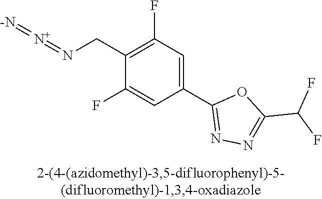 |
| Intermediate L | 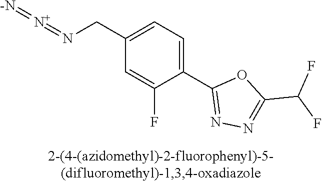 |
| Intermediate M | 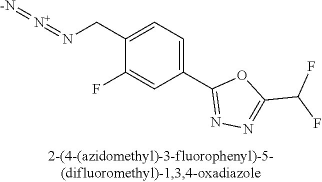 |
| Intermediate N | 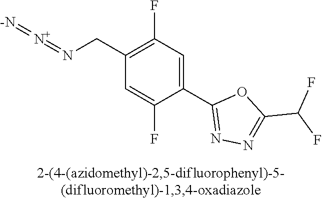 |
| Intermediate O | 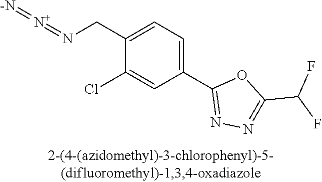 |
| Intermediate Q | 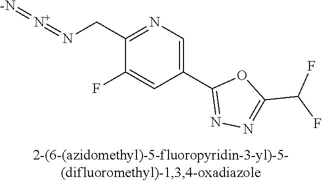 |

### Example 4. Synthesis of 2-(4-((5-(benzo[b]thiophen-3-yl)-2H-tetrazol-2-yl)methyl)phenyl)-5-(difluoromethyl)-1,3,4-oxadiazole (Compd. 207) and of 2-(4-((5-(benzo[b]thiophen-3-yl)-1H-tetrazol-1-yl)methyl)phenyl)-5-(difluoromethyl)-1,3,4-oxadiazole (Compd. 288)

Step A

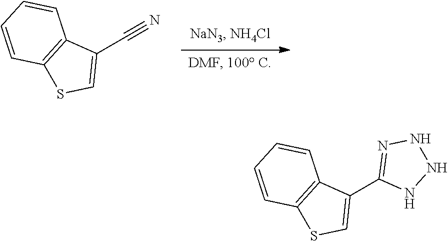

A mixture of Benzo[b]thiophene-3-carbonitrile (55 mg, 0.34 mmol, 1 equiv.), sodium azide (2 equiv.) and ammonium chloride (2 equiv.) in 1.5 mL DMF was stirred at 110° C. overnight. The reaction mixture was cooled to 0° C. and diluted with water. Precipitation occurred. Solid was filtered and washed with water 5 times. Product was used for the next step without any further purification.

Step B

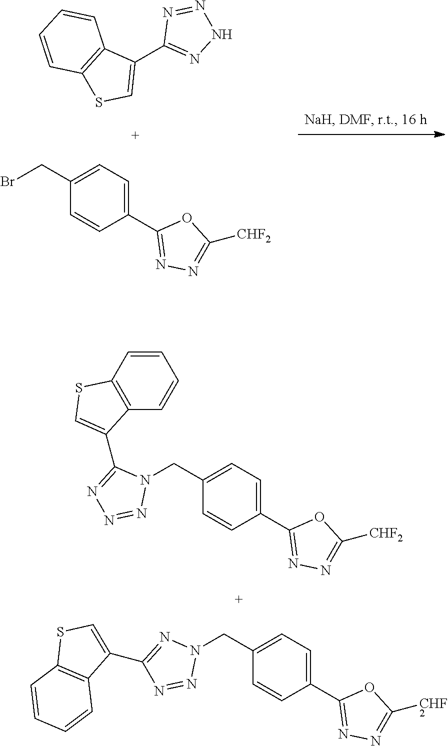 [SMILES: c1ccc2c(-c3nn[nH]n3)csc2c1 ; "NaH, DMF, r.t., 16 h" ; FC(F)c1nnc(-c2ccc(CBr)cc2)o1 ; FC(F)c1nnc(-c2ccc(Cn3nnnc3-c3csc4ccccc34)cc2)o1 ; FC(F)c1nnc(-c2ccc(Cn3nnc(-c4csc5ccccc45)n3)cc2)o1]

A mixture of 5-(1-benzothiophen-3-yl)-2H-tetrazole (65 mg, 0.321 mmol, 1 equiv.) and sodium hydride (1.1 equiv.) in 1 mL of DMF was stirred at r.t. for 1 h. 2-[4-(bromomethyl)phenyl]-5-(difluoromethyl)-1,3,4-oxadiazole (Intermediate B, 111.5 mg, 1.2 equiv.) was added and the reaction mixture was stirred overnight. Full conversion was observed by LC-MS. The reaction mixture was diluted with water. Precipitation occurred. The solid was recovered by filtration and submitted to prep-HPLC. 47.2 mg of 2-[4-[[5-(1-benzothiophen-3-yl)tetrazol-2-yl]methyl]phenyl]-5-(difluoromethyl)-1,3,4-oxadiazole (0.115 mmol, m/z 452.06 [M+ACN+H]⁺) and 8 mg of 2-[4-[[5-(1-benzothiophen-3-yl)tetrazol-1-yl]methyl]phenyl]-5-(difluoromethyl)-1,3,4-oxadiazole (0.019 mmol, m/z 452.06 [M+ACN+H]⁺) were obtained.

Following compounds were synthesized according to the same procedure:

| Compd. | Structure | m/z [MH+] |
|---|---|---|
| 4 | 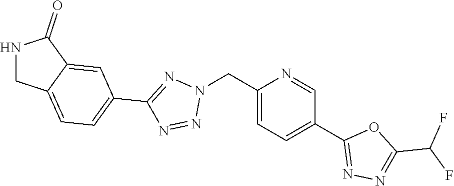 [SMILES: O=C1NCc2ccc(-c3nnn(Cc4ccc(-c5nnc(C(F)F)o5)cn4)n3)cc21] | 411.5 |
| 35 | 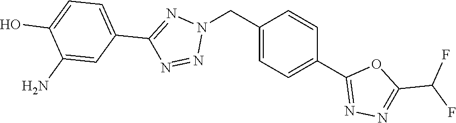 [SMILES: Nc1cc(-c2nnn(Cc3ccc(-c4nnc(C(F)F)o4)cc3)n2)ccc1O] | 385.8 |
| 38 | 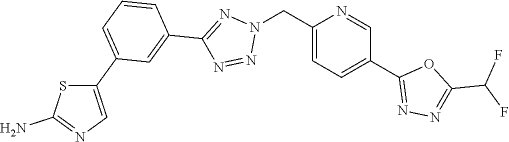 [SMILES: Nc1ncc(-c2cccc(-c3nnn(Cc4ccc(-c5nnc(C(F)F)o5)cn4)n3)c2)s1] | 454.94 |
| 40 | 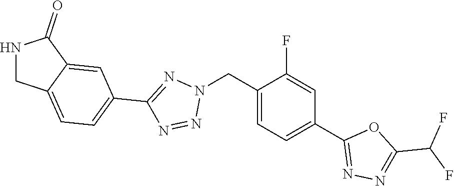 [SMILES: O=C1NCc2ccc(-c3nnn(Cc4ccc(-c5nnc(C(F)F)o5)cc4F)n3)cc21] | 428.3 |
| 45 | 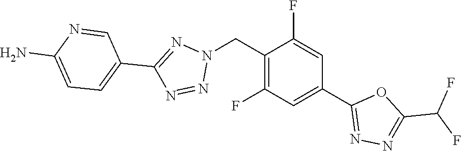 [SMILES: Nc1ccc(-c2nnn(Cc3c(F)cc(-c4nnc(C(F)F)o4)cc3F)n2)cn1] | 407.04 |
| 55 | 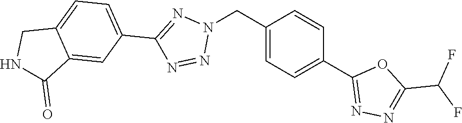 [SMILES: O=C1NCc2ccc(-c3nnn(Cc4ccc(-c5nnc(C(F)F)o5)cc4)n3)cc21] | 451.16* |
| 56 | 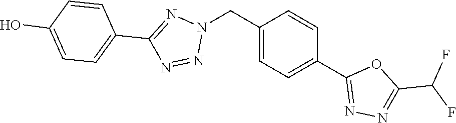 [SMILES: Oc1ccc(-c2nnn(Cc3ccc(-c4nnc(C(F)F)o4)cc3)n2)cc1] | 371.8 |
| 57 | 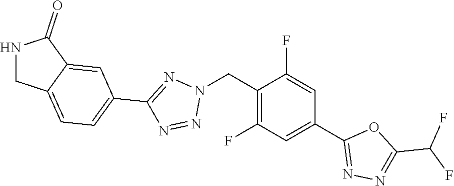 [SMILES: O=C1NCc2ccc(-c3nnn(Cc4c(F)cc(-c5nnc(C(F)F)o5)cc4F)n3)cc21] | 487.0* |
| 58 | 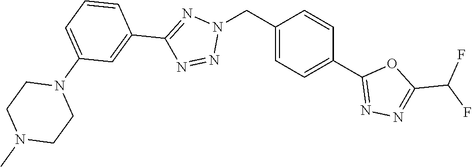 [SMILES: CN1CCN(c2cccc(-c3nnn(Cc4ccc(-c5nnc(C(F)F)o5)cc4)n3)c2)CC1] | 453.21 |
| 64 | 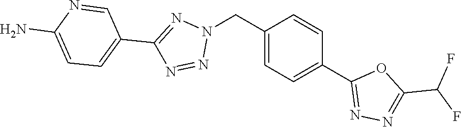 [SMILES: Nc1ccc(-c2nnn(Cc3ccc(-c4nnc(C(F)F)o4)cc3)n2)cn1] | 371.5 |
| 65 | 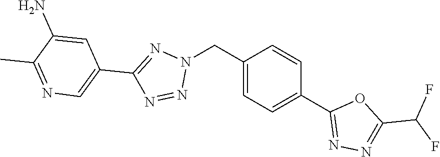 [SMILES: Cc1ncc(-c2nnn(Cc3ccc(-c4nnc(C(F)F)o4)cc3)n2)cc1N] | 385.5 |
| 72 | 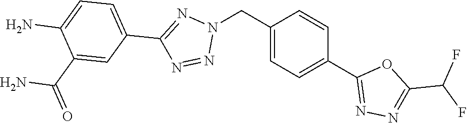 [SMILES: NC(=O)c1cc(-c2nnn(Cc3ccc(-c4nnc(C(F)F)o4)cc3)n2)ccc1N] | 412.85 |
| 76 | 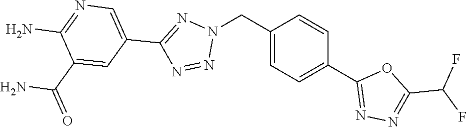 [SMILES: NC(=O)c1cc(-c2nnn(Cc3ccc(-c4nnc(C(F)F)o4)cc3)n2)cnc1N] | 414.07 |
| 77 | 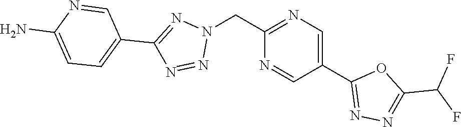 [SMILES: Nc1ccc(-c2nnn(Cc3ncc(-c4nnc(C(F)F)o4)cn3)n2)cn1] | 373.01 |
| 79 | 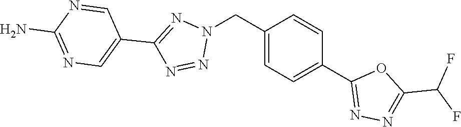 [SMILES: Nc1ncc(-c2nnn(Cc3ccc(-c4nnc(C(F)F)o4)cc3)n2)cn1] | 371.8 |
| 82 | 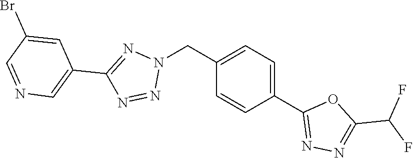 [SMILES: FC(F)c1nnc(-c2ccc(Cn3nnc(-c4cncc(Br)c4)n3)cc2)o1] | 434.2 |
| 83 | 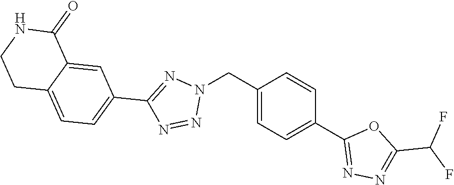 [SMILES: O=C1NCCc2ccc(-c3nnn(Cc4ccc(-c5nnc(C(F)F)o5)cc4)n3)cc21] | 424.08 |
| 87 | 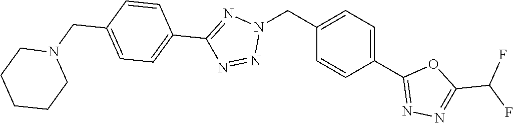 [SMILES: FC(F)c1nnc(-c2ccc(Cn3nnc(-c4ccc(CN5CCCCC5)cc4)n3)cc2)o1] | 452.19 |
| 90 | 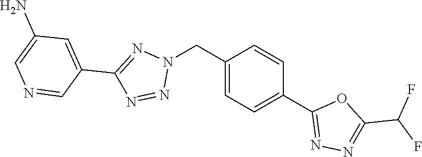 [SMILES: Nc1cncc(-c2nnn(Cc3ccc(-c4nnc(C(F)F)o4)cc3)n2)c1] | 370.96 |
| 91 | 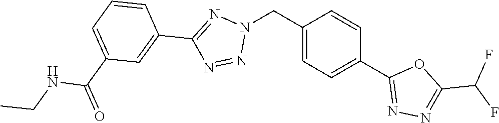 [SMILES: CCNC(=O)c1cccc(-c2nnn(Cc3ccc(-c4nnc(C(F)F)o4)cc3)n2)c1] | 426.09 |
| 97 | 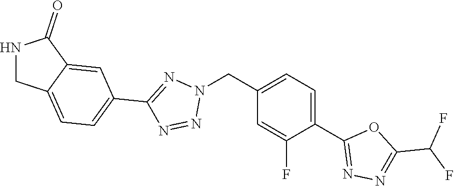 [SMILES: O=C1NCc2ccc(-c3nnn(Cc4ccc(-c5nnc(C(F)F)o5)c(F)c4)n3)cc21] | 428.09 |
| 99 | 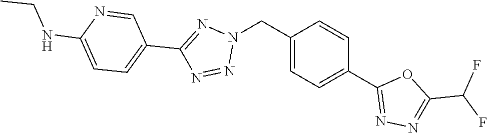 [SMILES: CCNc1ccc(-c2nnn(Cc3ccc(-c4nnc(C(F)F)o4)cc3)n2)cn1] | 399.26 |
| 107 | 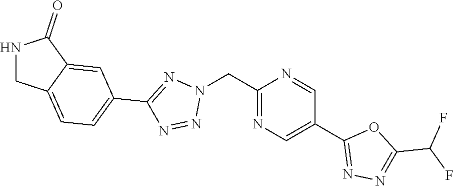 [SMILES: O=C1NCc2ccc(-c3nnn(Cc4ncc(-c5nnc(C(F)F)o5)cn4)n3)cc21] | 412.5 |
| 113 | 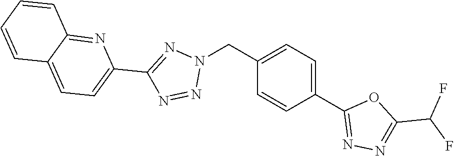 [SMILES: FC(F)c1nnc(-c2ccc(Cn3nnc(-c4ccc5ccccc5n4)n3)cc2)o1] | 406.15 |
| 116 | 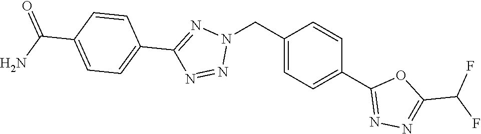 [SMILES: NC(=O)c1ccc(-c2nnn(Cc3ccc(-c4nnc(C(F)F)o4)cc3)n2)cc1] | 398.15 |
| 118 | 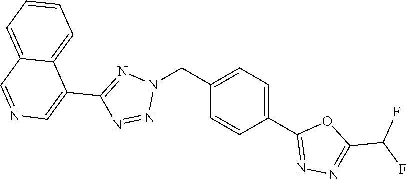 [SMILES: FC(F)c1nnc(-c2ccc(Cn3nnc(-c4cncc5ccccc45)n3)cc2)o1] | 406.16 |
| 127 | 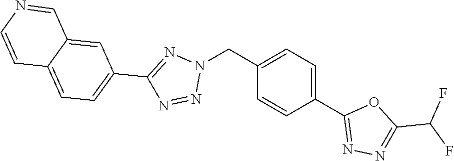 [SMILES: FC(F)c1nnc(-c2ccc(Cn3nnc(-c4ccc5ccncc5c4)n3)cc2)o1] | 405.96 |
| 128 | 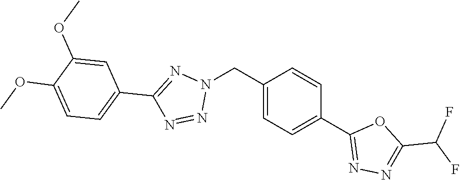 [SMILES: COc1ccc(-c2nnn(Cc3ccc(-c4nnc(C(F)F)o4)cc3)n2)cc1OC] | 415.09 |
| 129 | 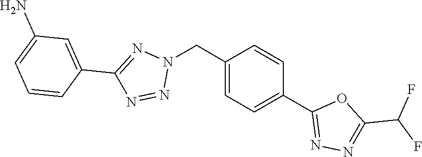 [SMILES: Nc1cccc(-c2nnn(Cc3ccc(-c4nnc(C(F)F)o4)cc3)n2)c1] | 370.06 |
| 133 | 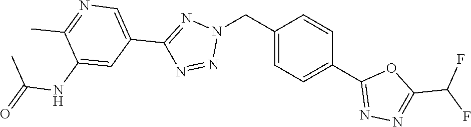 [SMILES: CC(=O)Nc1cc(-c2nnn(Cc3ccc(-c4nnc(C(F)F)o4)cc3)n2)cnc1C] | 427.02 |
| 140 | 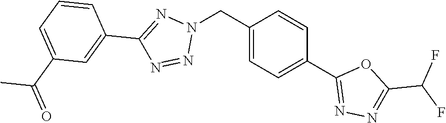 [SMILES: CC(=O)c1cccc(-c2nnn(Cc3ccc(-c4nnc(C(F)F)o4)cc3)n2)c1] | 397.5 |
| 147 | 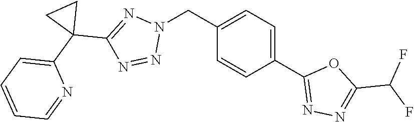 [SMILES: FC(F)c1nnc(-c2ccc(Cn3nnc(C4(c5ccccn5)CC4)n3)cc2)o1] | 395.97 |
| 150 | 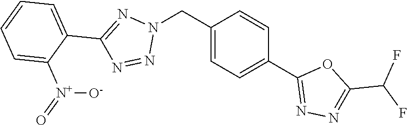 [SMILES: O=[N+]([O-])c1ccccc1-c1nnn(Cc2ccc(-c3nnc(C(F)F)o3)cc2)n1] | 400.02 |
| 153 | 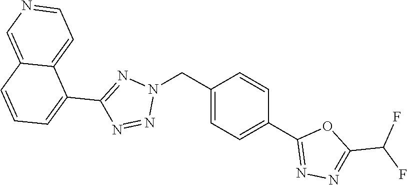 [SMILES: FC(F)c1nnc(-c2ccc(Cn3nnc(-c4cccc5cnccc45)n3)cc2)o1] | 405.97 |
| 167 | 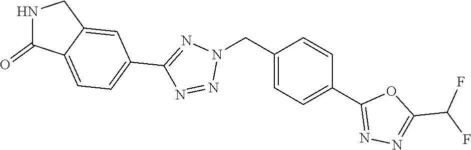 [SMILES: O=C1NCc2cc(-c3nnn(Cc4ccc(-c5nnc(C(F)F)o5)cc4)n3)ccc21] | 410.3 |
| 168 | 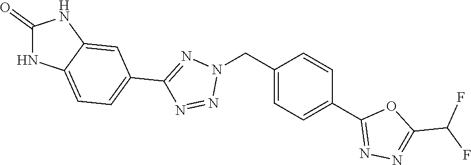 [SMILES: O=c1[nH]c2ccc(-c3nnn(Cc4ccc(-c5nnc(C(F)F)o5)cc4)n3)cc2[nH]1] | 410.85 |
| 181 | 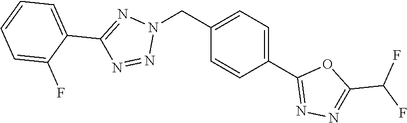 [SMILES: Fc1ccccc1-c1nnn(Cc2ccc(-c3nnc(C(F)F)o3)cc2)n1] | 373.05 |
| 187 | 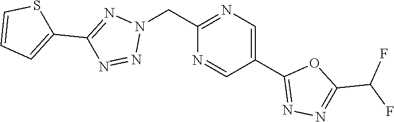 [SMILES: FC(F)c1nnc(-c2cnc(Cn3nnc(-c4cccs4)n3)nc2)o1] | 636.14 |
| 188 | 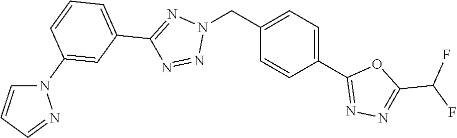 [SMILES: FC(F)c1nnc(-c2ccc(Cn3nnc(-c4cccc(-n5cccn5)c4)n3)cc2)o1] | 420.84 |
| 200 | 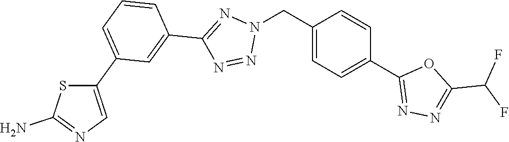 [SMILES: Nc1ncc(-c2cccc(-c3nnn(Cc4ccc(-c5nnc(C(F)F)o5)cc4)n3)c2)s1] | 452.9 |
| 201 | 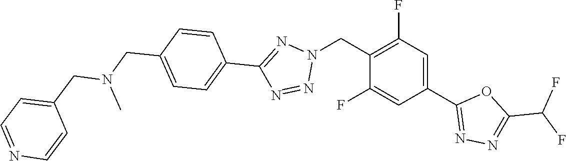 [SMILES: CN(Cc1ccncc1)Cc1ccc(-c2nnn(Cc3c(F)cc(-c4nnc(C(F)F)o4)cc3F)n2)cc1] | 525.16 |
| 203 | 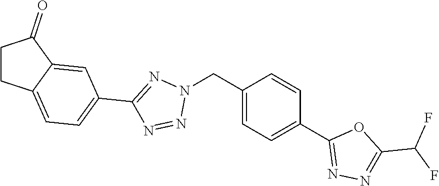 [SMILES: O=C1CCc2ccc(-c3nnn(Cc4ccc(-c5nnc(C(F)F)o5)cc4)n3)cc21] | 409.3 |
| 204 | 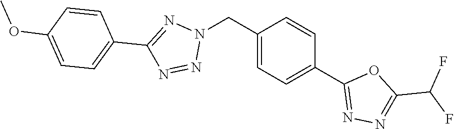 [SMILES: COc1ccc(-c2nnn(Cc3ccc(-c4nnc(C(F)F)o4)cc3)n2)cc1] | 385.08 |
| 208 | 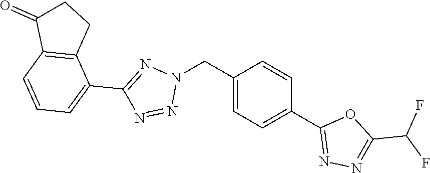 [SMILES: O=C1CCc2c1cccc2-c1nnn(Cc2ccc(-c3nnc(C(F)F)o3)cc2)n1] | 409.5 |
| 210 | 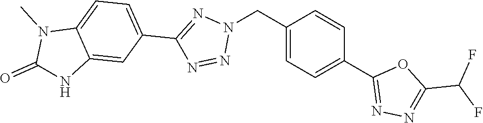 [SMILES: Cn1c(=O)[nH]c2cc(-c3nnn(Cc4ccc(-c5nnc(C(F)F)o5)cc4)n3)ccc21] | 425.04 |
| 212 | 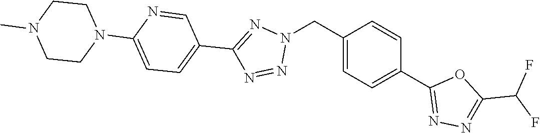 [SMILES: CN1CCN(c2ccc(-c3nnn(Cc4ccc(-c5nnc(C(F)F)o5)cc4)n3)cn2)CC1] | 454.04 |
| 213 | 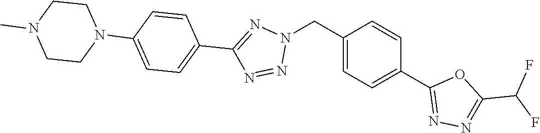 [SMILES: CN1CCN(c2ccc(-c3nnn(Cc4ccc(-c5nnc(C(F)F)o5)cc4)n3)cc2)CC1] | 453.05 |
| 215 | 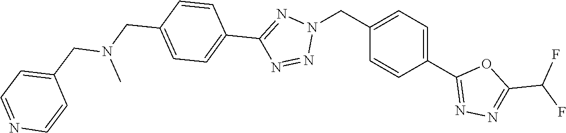 [SMILES: CN(Cc1ccncc1)Cc1ccc(-c2nnn(Cc3ccc(-c4nnc(C(F)F)o4)cc3)n2)cc1] | 489.16 |
| 219 | 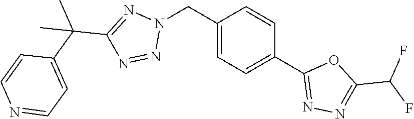 [SMILES: CC(C)(c1ccncc1)c1nnn(Cc2ccc(-c3nnc(C(F)F)o3)cc2)n1] | 398.03 |
| 220 | 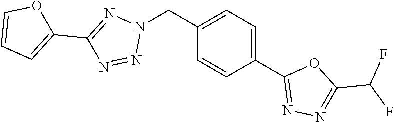 [SMILES: FC(F)c1nnc(-c2ccc(Cn3nnc(-c4ccco4)n3)cc2)o1] | 345.5 |
| 246 | 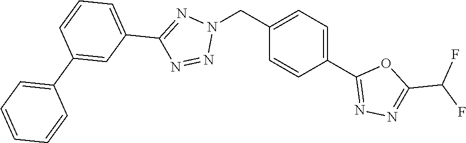 [SMILES: FC(F)c1nnc(-c2ccc(Cn3nnc(-c4cccc(-c5ccccc5)c4)n3)cc2)o1] | 431.3 |
| 249 | 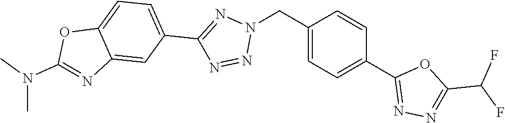 [SMILES: CN(C)c1nc2cc(-c3nnn(Cc4ccc(-c5nnc(C(F)F)o5)cc4)n3)ccc2o1] | 438.92 |
| 251 | 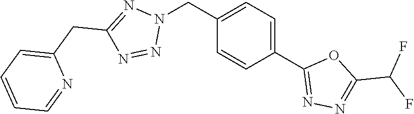 [SMILES: FC(F)c1nnc(-c2ccc(Cn3nnc(Cc4ccccn4)n3)cc2)o1] | 369.94 |
| 256 | 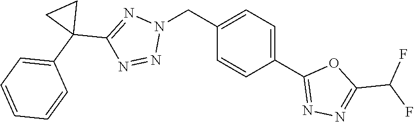 [SMILES: FC(F)c1nnc(-c2ccc(Cn3nnc(C4(c5ccccc5)CC4)n3)cc2)o1] | 394.86 |
| 263 | 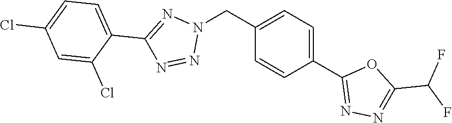 [SMILES: FC(F)c1nnc(-c2ccc(Cn3nnc(-c4ccc(Cl)cc4Cl)n3)cc2)o1] | 423.1 |
| 278 | 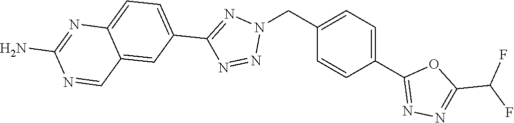 [SMILES: Nc1ncc2cc(-c3nnn(Cc4ccc(-c5nnc(C(F)F)o5)cc4)n3)ccc2n1] | 422.01 |
| 287 | 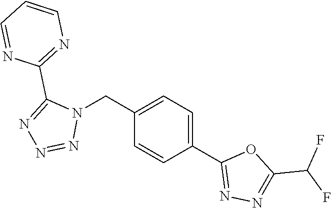 [SMILES: FC(F)c1nnc(-c2ccc(Cn3nnnc3-c3ncccn3)cc2)o1] | 357.1 |
| 289 | 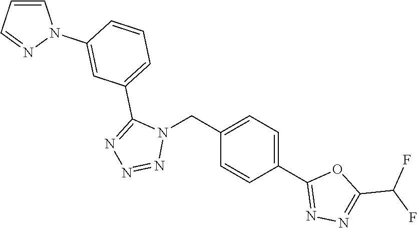 [SMILES: FC(F)c1nnc(-c2ccc(Cn3nnnc3-c3cccc(-n4cccn4)c3)cc2)o1] | 420.85 |
| 290 | 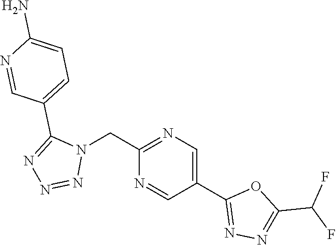 [SMILES: Nc1ccc(-c2nnnn2Cc2ncc(-c3nnc(C(F)F)o3)cn2)cn1] | 373.01 |
| 298 | 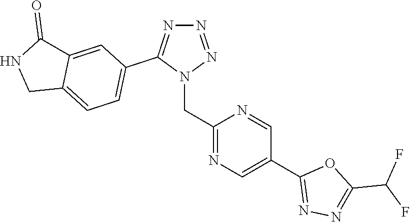 [SMILES: O=C1NCc2ccc(-c3nnnn3Cc3ncc(-c4nnc(C(F)F)o4)cn3)cc21] | 412.21 |
| 299 | 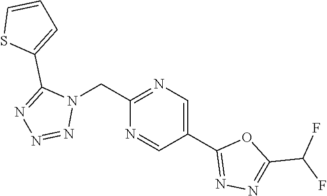 [SMILES: FC(F)c1nnc(-c2cnc(Cn3nnnc3-c3cccs3)nc2)o1] | 362.97 |
| 388 | 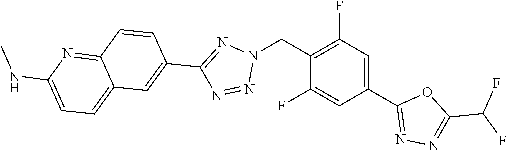 [SMILES: CNc1ccc2cc(-c3nnn(Cc4c(F)cc(-c5nnc(C(F)F)o5)cc4F)n3)ccc2n1] | 471.16 |
| 396 | 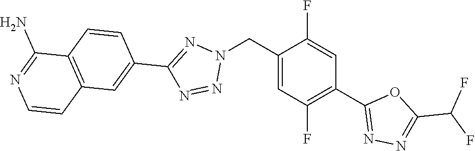 [SMILES: Nc1nccc2cc(-c3nnn(Cc4cc(F)c(-c5nnc(C(F)F)o5)cc4F)n3)ccc12] | 457.16 |
| 397 | 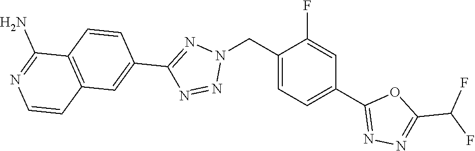 [SMILES: Nc1nccc2cc(-c3nnn(Cc4ccc(-c5nnc(C(F)F)o5)cc4F)n3)ccc12] | 439.17 |
| 398 | 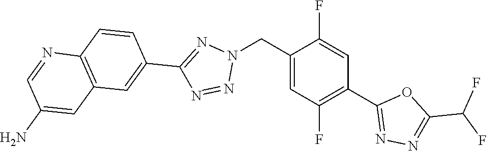 [SMILES: Nc1cnc2ccc(-c3nnn(Cc4cc(F)c(-c5nnc(C(F)F)o5)cc4F)n3)cc2c1] | 457.16 |
| 400 | 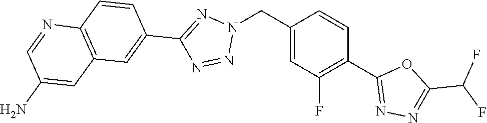 [SMILES: Nc1cnc2ccc(-c3nnn(Cc4ccc(-c5nnc(C(F)F)o5)c(F)c4)n3)cc2c1] | 439.18 |
| 401 | 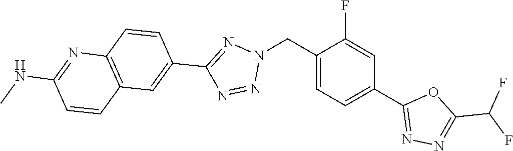 [SMILES: CNc1ccc2cc(-c3nnn(Cc4ccc(-c5nnc(C(F)F)o5)cc4F)n3)ccc2n1] | 452.91 |
| 403 | 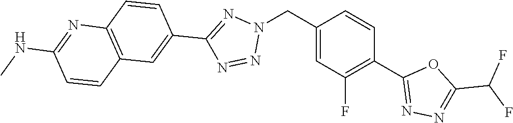 [SMILES: CNc1ccc2cc(-c3nnn(Cc4ccc(-c5nnc(C(F)F)o5)c(F)c4)n3)ccc2n1] | 453.19 |
| 407 | 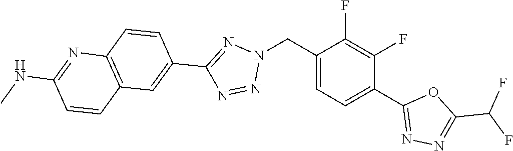 [SMILES: CNc1ccc2cc(-c3nnn(Cc4ccc(-c5nnc(C(F)F)o5)c(F)c4F)n3)ccc2n1] | 471.13 |
| 409 | 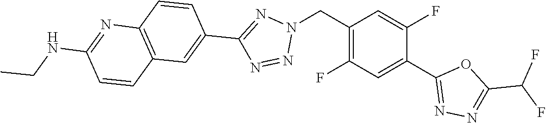 [SMILES: CCNc1ccc2cc(-c3nnn(Cc4cc(F)c(-c5nnc(C(F)F)o5)cc4F)n3)ccc2n1] | 485.2 |
| 410 | 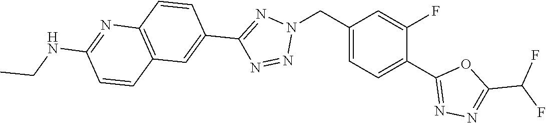 [SMILES: CCNc1ccc2cc(-c3nnn(Cc4ccc(-c5nnc(C(F)F)o5)c(F)c4)n3)ccc2n1] | 467.17 |
| 422 | 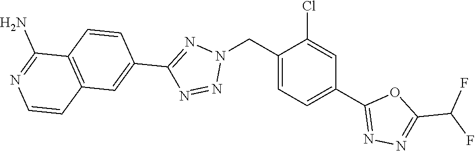 [SMILES: Nc1nccc2cc(-c3nnn(Cc4ccc(-c5nnc(C(F)F)o5)cc4Cl)n3)ccc12] | 455.39 |
| 423 | 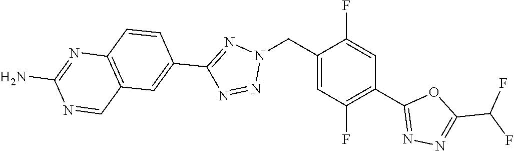 [SMILES: Nc1ncc2cc(-c3nnn(Cc4cc(F)c(-c5nnc(C(F)F)o5)cc4F)n3)ccc2n1] | 458.13 |
| 424 | 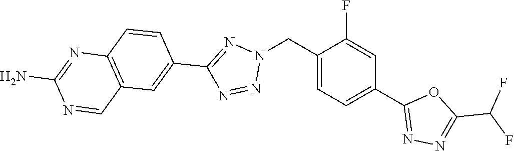 [SMILES: Nc1ncc2cc(-c3nnn(Cc4ccc(-c5nnc(C(F)F)o5)cc4F)n3)ccc2n1] | 440.18 |
| 425 | 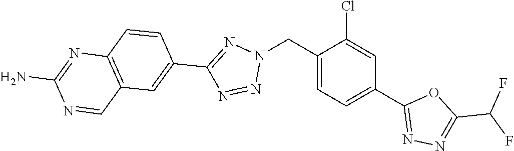 [SMILES: Nc1ncc2cc(-c3nnn(Cc4ccc(-c5nnc(C(F)F)o5)cc4Cl)n3)ccc2n1] | 456.13 |
| 426 | 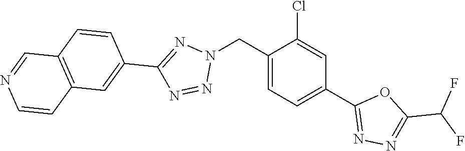 [SMILES: FC(F)c1nnc(-c2ccc(Cn3nnc(-c4ccc5cnccc5c4)n3)c(Cl)c2)o1] | 440.12 |
| 427 | 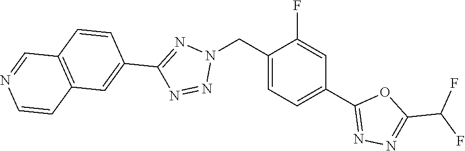 [SMILES: Fc1cc(-c2nnc(C(F)F)o2)ccc1Cn1nnc(-c2ccc3cnccc3c2)n1] | 423.92 |
| 428 | 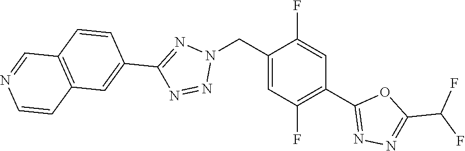 [SMILES: Fc1cc(-c2nnc(C(F)F)o2)c(F)cc1Cn1nnc(-c2ccc3cnccc3c2)n1] | 442.16 |
| 429 | 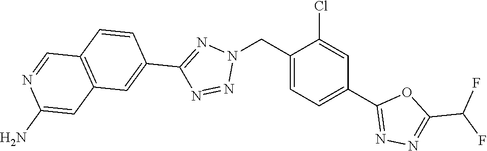 [SMILES: Nc1cc2cc(-c3nnn(Cc4ccc(-c5nnc(C(F)F)o5)cc4Cl)n3)ccc2cn1] | 455.13 |
| 430 | 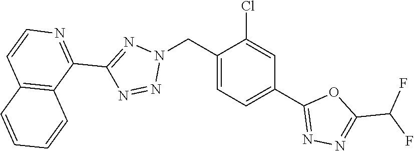 [SMILES: FC(F)c1nnc(-c2ccc(Cn3nnc(-c4nccc5ccccc45)n3)c(Cl)c2)o1] | 440.12 |
| 431 | 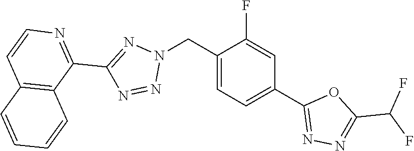 [SMILES: Fc1cc(-c2nnc(C(F)F)o2)ccc1Cn1nnc(-c2nccc3ccccc23)n1] | 424.17 |
| 432 | 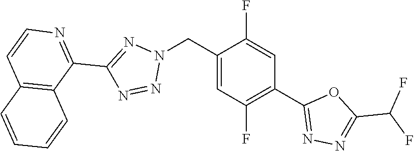 [SMILES: Fc1cc(-c2nnc(C(F)F)o2)c(F)cc1Cn1nnc(-c2nccc3ccccc23)n1] | 442.14 |
| 504 | 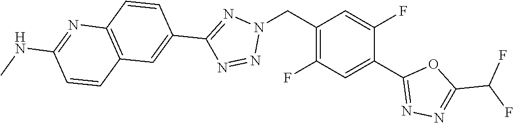 [SMILES: CNc1ccc2cc(-c3nnn(Cc4cc(F)c(-c5nnc(C(F)F)o5)cc4F)n3)ccc2n1] | 470.89 |

* [M + ACN + H]⁺ was observed.

### Example 5. Synthesis of 5-(2-((5-(5-(difluoromethyl)-1,3,4-oxadiazol-2-yl)pyridin-2-yl)methyl)-2H-tetrazol-5-yl)benzo[d]oxazol-2-amine. (Compd. 7)

Step A

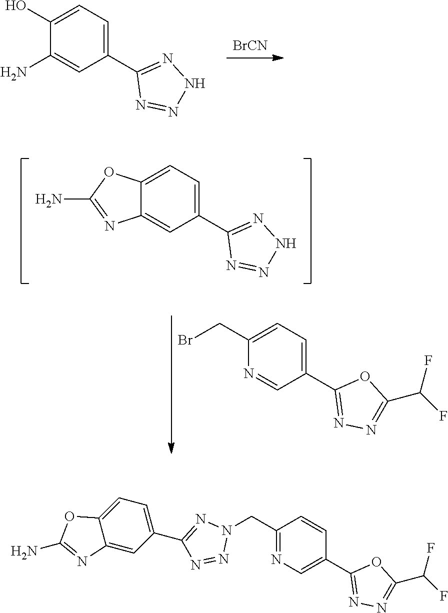

2-amino-4-(2H-tetrazol-5-yl)phenol (150 mg, 0.85 mmol, 1 equiv.) and cyanogen bromide (89.7 mg, 0.85 mmol, 1 equiv.) were dissolved in DMF (5 mL) and the reaction mixture was stirred overnight at 60° C. Full conversion to benzoxazole was observed by LC-MS. 2-[6-(bromomethyl)pyridin-3-yl]-5-(difluoromethyl)-1,3,4-oxadiazole (Intermediate A, 245.6 mg, 0.85 mmol, 1 equiv.) and potassium carbonate (234 mg, 1.69 mmol, 2 equiv.) were added and the reaction mixture was stirred at r.t. overnight. Full conversion to desired product was observed by LC-MS. The reaction mixture was diluted with water and the product was extracted with EtOAc. Organic phase was washed with aqueous sodium bicarbonate and brine, dried over Na₂SO₄, filtered and evaporated. Residual DMF was diluted with EtOAc. Precipitation occurred and solid was filtered. After drying, solid was suspended in MeOH and freeze-dried, affording pure product (72.1 mg, 20.12% yield, m/z 412.34 [MH+]).

Following compounds were synthesized according to the same procedure:

| Compd. | structure | m/z [MH+] |
|---|---|---|
| 11 | 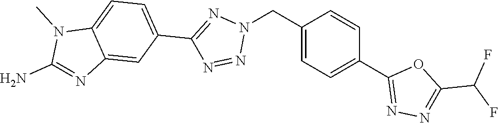 [SMILES: Cn1c(N)nc2cc(-c3nnn(Cc4ccc(-c5nnc(C(F)F)o5)cc4)n3)ccc21] | 423.99 |
| 252 | 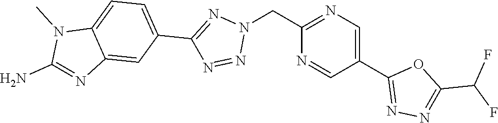 [SMILES: Cn1c(N)nc2cc(-c3nnn(Cc4ncc(-c5nnc(C(F)F)o5)cn4)n3)ccc21] | 425.95 |
| 291 | 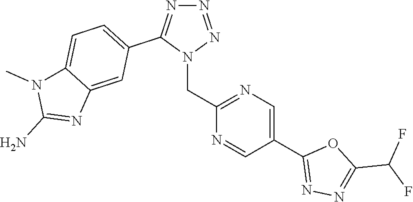 [SMILES: Cn1c(N)nc2cc(-c3nnnn3Cc3ncc(-c4nnc(C(F)F)o4)cn3)ccc21] | 426.1 |
| 348 | 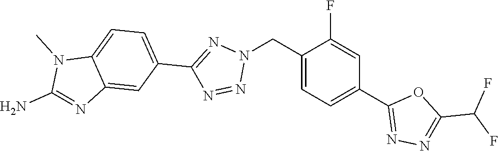 [SMILES: Cn1c(N)nc2cc(-c3nnn(Cc4ccc(-c5nnc(C(F)F)o5)cc4F)n3)ccc21] | 442.12 |
| 370 | 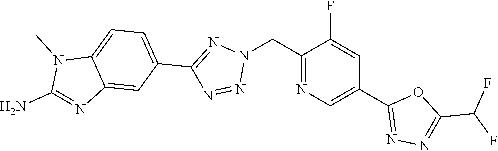 [SMILES: Cn1c(N)nc2cc(-c3nnn(Cc4ncc(-c5nnc(C(F)F)o5)cc4F)n3)ccc21] | 443.1 |
| 371 | 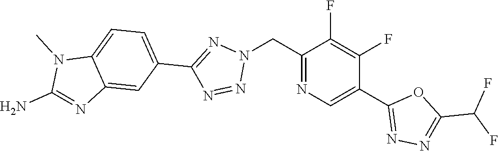 [SMILES: Cn1c(N)nc2cc(-c3nnn(Cc4ncc(-c5nnc(C(F)F)o5)c(F)c4F)n3)ccc21] | 460.09 |
| 372 | 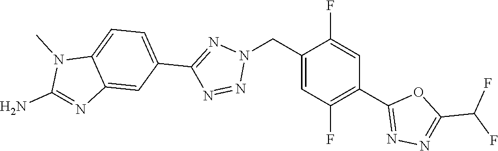 [SMILES: Cn1c(N)nc2cc(-c3nnn(Cc4cc(F)c(-c5nnc(C(F)F)o5)cc4F)n3)ccc21] | 460.07 |
| 373 | 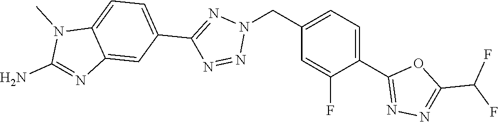 [SMILES: Cn1c(N)nc2cc(-c3nnn(Cc4ccc(-c5nnc(C(F)F)o5)c(F)c4)n3)ccc21] | 442.11 |
| 374 | 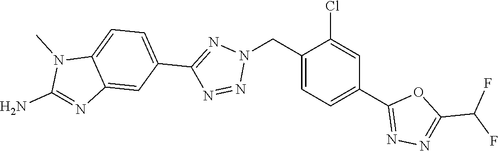 [SMILES: Cn1c(N)nc2cc(-c3nnn(Cc4ccc(-c5nnc(C(F)F)o5)cc4Cl)n3)ccc21] | 458.06 |

### Example 6. Synthesis of 4-(5-(3-(2-((5-(5-(difluoromethyl)-1,3,4-oxadiazol-2-yl)pyridin-2-yl)methyl)-2H-tetrazol-5-yl)phenyl)thiazol-2-yl)morpholine. (Compd. 130)

Step A

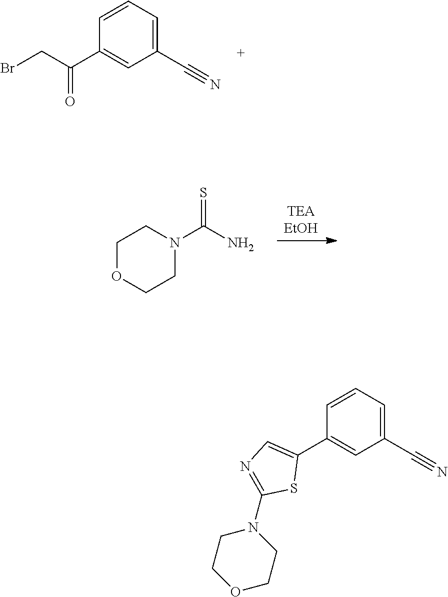 [SMILES: N#Cc1cccc(C(=O)CBr)c1 ; NC(=S)N1CCOCC1 ; "TEAEtOH" ; N#Cc1cccc(-c2cnc(N3CCOCC3)s2)c1]

A solution of 3-(2-bromoacetyl)benzonitrile (500 mg, 1.23 mmol, 1 equiv.) and morpholine-4-carbothioamide (326.19 mg, 2.23 mmol, 1 equiv.) in ethanol (10 mL) was refluxed for 2 h. The solvent was removed under reduced pressure. The product, 3-(2-morpholin-4-yl-1,3-thiazol-5-yl)benzonitrile, was obtained as a white solid and used without further purification.

Step B

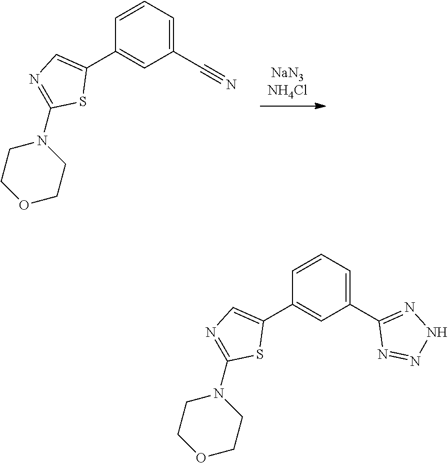 [SMILES: N#Cc1cccc(-c2cnc(N3CCOCC3)s2)c1 ; "NaN3NH4Cl" ; c1cc(-c2nn[nH]n2)cc(-c2cnc(N3CCOCC3)s2)c1]

A mixture of 3-(2-morpholin-4-yl-1,3-thiazol-5-yl)benzonitrile (605.4 mg, 2.23 mmol, 1 equiv.), sodium azide (290.1 mg, 4.46 mmol, 2 equiv.) and ammonium chloride (119.3 mg, 2.23 mmol, 1 equiv.) in DMF (10 mL) was stirred at 90° C. overnight. Additional portions of sodium azide (1.0 equiv.) and ammonium chloride (1.0 equiv.) were added, in order to achieve complete conversion. The reaction mixture was stirred for 12 h at 90° C., then it was cooled down to r.t. and concentrated by rotary evaporation. Reaction mixture was then diluted with water, cooled to 0° C. Acetic acid was added dropwise. Precipitation occurred and the solid was collected by filtration, dried in vacuo and used in the next step without further purification.

Step C

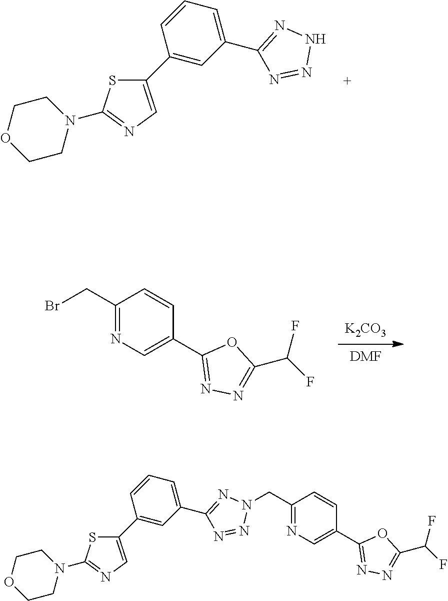

2-[6-(bromomethyl)pyridin-3-yl]-5-(difluoromethyl)-1,3,4-oxadiazole (Intermediate A, 92.27 mg, 0.32 mmol, 1 equiv.) was added to a solution of 4-[5-[3-(2H-tetrazol-5-yl)phenyl]-1,3-thiazol-2-yl]morpholine (100 mg, 0.32 mmol, 1 equiv.) and potassium carbonate (87.93 mg, 0.64 mmol, 2 equiv.) in DMF (5 mL). The reaction mixture was stirred at r.t. overnight. Full conversion was verified by LC-MS. Reaction mixture was diluted with water and precipitation occurred. Solid was filtered and purified by prep-HPLC, affording pure product (87 mg, 0.16 mmol, 25.7% yield, m/z 523.94 [MH+]). Following compound was synthesized according to the same procedure:

| Compd. | Structure | m/z [MH+] |
|---|---|---|
| 253 | 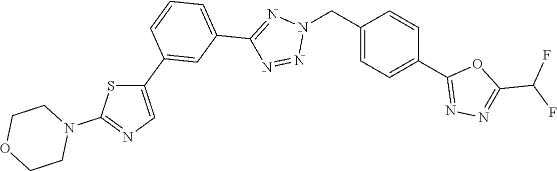 [SMILES: FC(F)c1nnc(-c2ccc(Cn3nnc(-c4cccc(-c5cnc(N6CCOCC6)s5)c4)n3)cc2)o1] | 522.94 |

### Example 7. Synthesis of 5-(2-(4-(5-(difluoromethyl)-1,3,4-oxadiazol-2-yl)benzyl)-2H-tetrazol-5-yl)benzo[d]oxazol-2-amine. (Compd. 152)

Step A

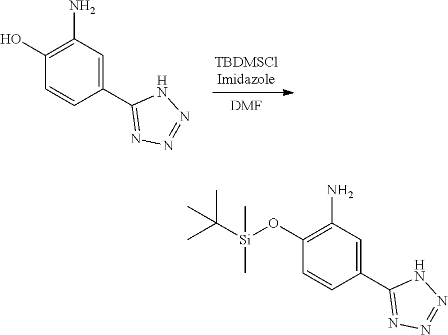

A solution of 2-amino-4-(2H-tetrazol-5-yl)phenol (500 mg, 2.82 mmol, 1 equiv.), tert-butylchlorodimethylsilane (680.61 mg, 4.5 mmol, 1.6 equiv.) and imidazole (345.86 mg, 5.08 mmol, 1.8 equiv.) in DMF (4 mL) was stirred overnight at r.t. Full conversion was observed by LC-MS. The reaction mixture was diluted with water and precipitation occurred. The solid product (690 mg, 2.37 mmol, 83.9% yield) was filtered, washed with n-hexane, dried and used without any purification for the next step.

Step B

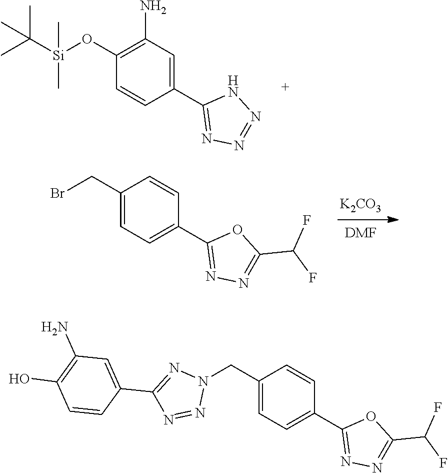

To a solution of 2-[cert-butyl(dimethyl)silyl]oxy-5-(2H-tetrazol-5-yl)aniline (120 mg, 0.41 mmol, 1 equiv.) and 2-[4-(bromomethyl)phenyl]-5-(difluoromethyl)-1,3,4-oxadiazole (Intermediate B, 130.9 mg, 0.45 mmol, 1.1 equiv.) in DMF (2 mL) potassium carbonate (114 mg, 0.824 mmol, 2 equiv.) was added and the reaction mixture was stirred at r.t. overnight. Full conversion was verified by LC-MS. Reaction mixture was diluted with water and the product was extracted with ethyl acetate. Organic phase was dried over Na₂SO₄, filtered and evaporated under reduced pressure. Crude was used for the next step without any purification.

Step C

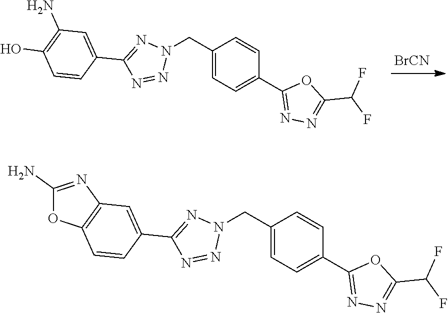

2-amino-4-[2-[[4-[5-(difluoromethyl)-1,3,4-oxadiazol-2-yl]phenyl]methyl]tetrazol-5-yl]phenol (96 mg, 0.197 mmol, 1 equiv.) and cyanogen bromide (22.98 mg, 0.217 mmol, 1.1 equiv.) were dissolved in EtOH (2 mL) and the reaction mixture was stirred at r.t. overnight. Full conversion to benzoxazole was observed by LC-MS. Solvent was evaporated under reduced pressure and crude was purified by LC-MS, affording 14 mg of pure product (0.034 mmol, 17.4% yield, m/z 411.06 [MH+]).

### Example 8. 5-(2-(4-(5-(difluoromethyl)-1,3,4-oxadiazol-2-yl)benzyl)-2H-tetrazol-5-yl)benzo[d]oxazol-2 (3H)-one. (Compd. 225)

Step A

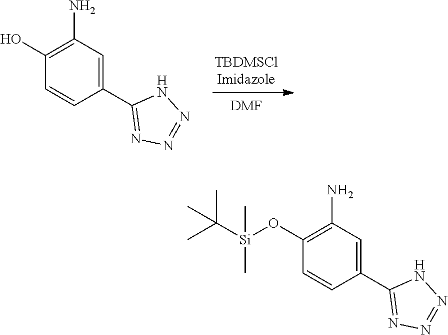

A solution of 2-amino-4-(2H-tetrazol-5-yl)phenol (500 mg, 2.82 mmol, 1 equiv.), tert-butylchlorodimethylsilane (680.61 mg, 4.5 mmol, 1.6 equiv.) and imidazole (345.86 mg, 5.08 mmol, 1.8 equiv.) in DMF (4 mL) was stirred overnight at r.t. Full conversion was observed by LC-MS. The reaction mixture was diluted with water and precipitation occurred. The solid product (690 mg, 2.37 mmol, 83.9% Yield) was filtered, washed with n-hexane, dried and used without any purification for the next step.

Step B

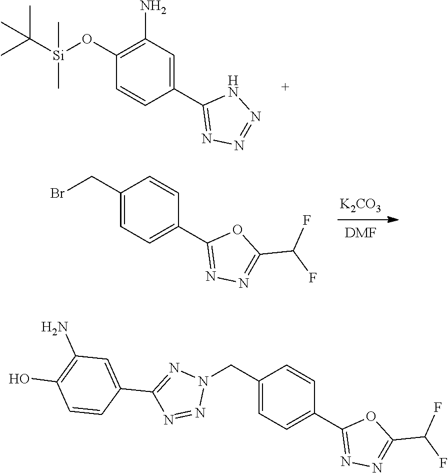

Potassium carbonate (113.82 mg, 0.824 mmol, 2 equiv.) was added to a solution of 2-[tert-butyl(dimethyl)silyl]oxy-5-(2H-tetrazol-5-yl)aniline (120 mg, 0.41 mmol, 1 equiv.) and 2-[4-(bromomethyl)phenyl]-5-(difluoromethyl)-1,3,4-oxadiazole (Intermediate B, 130.9 mg, 0.45 mmol, 1.1 equiv.) in DMF (2 mL), and the resulting mixture was stirred at r.t. overnight. Full conversion was verified by LC-MS. Reaction mixture was diluted with water and the product was extracted with ethyl acetate. Organic phase was dried over Na₂SO₄, filtered and evaporated under reduced pressure. Crude was used for the next step without any purification.

Step C

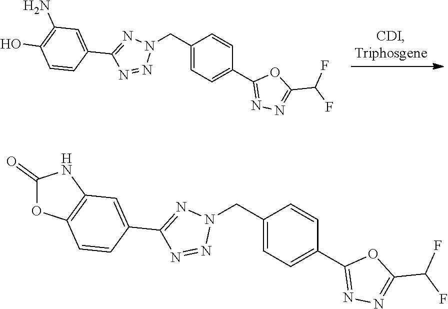 [SMILES: Nc1cc(-c2nnn(Cc3ccc(-c4nnc(C(F)F)o4)cc3)n2)ccc1O ; "CDI,Triphosgene" ; O=c1[nH]c2cc(-c3nnn(Cc4ccc(-c5nnc(C(F)F)o5)cc4)n3)ccc2o1]

1,1′-Carbonyldiimidazole (35.18 mg, 0.217 mmol, 1.1 equiv.) was added to a solution of 2-amino-4-[2-[[4-[5-(difluoromethyl)-1,3,4-oxadiazol-2-yl]phenyl]methyl]tetrazol-5-yl]phenol (95 mg, 0.197 mmol, 1 equiv.) in ACN (2 mL). The reaction mixture was stirred at 60° C. After one night only 10% conversion was observed by LC-MS. 2 additional equivalents of CDI were added. After two hours of stirring at 100° C., triphosgene (29.26 mg, 0.099 mmol, 0.5 equiv.) was added. The reaction mixture was stirred for 1 h at 80° C. Full conversion was observed. Solvent was evaporated under reduced pressure and crude was purified by prep-HPLC (13.9 mg, 0.034 mmol, 17.05% yield, m/z 409.7 [M−H]).

### Example 9. Synthesis of (3-(2-((5-(5-(difluoromethyl)-1,3,4-oxadiazol-2-yl)pyridin-2-yl)methyl)-2H-tetrazol-5-yl)phenyl)(morpholino)methanone. (Compd. 69)

Step A

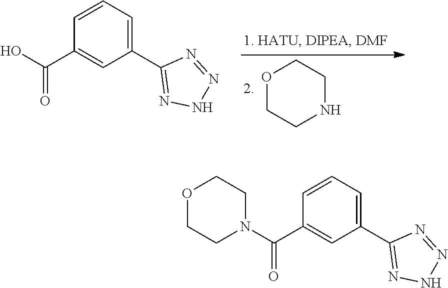 [SMILES: O=C(O)c1cccc(-c2nn[nH]n2)c1 ; C1COCCN1 ; O=C(c1cccc(-c2nn[nH]n2)c1)N1CCOCC1]

A mixture of 3-(1H-tetrazol-5-yl)benzoic acid (1.4 g, 7.4 mmol, 1 equiv.), HATU (4.2 g, 11 mmol, 1.5 equiv.) and DIPEA (3.2 mL, 18.4 mmol, 2.5 equiv.) in 12 mL of DMF was stirred at r.t. for 1 hour. A. Then morpholine (705.5 mg, 8 mmol, 1.1 equiv.) was added, and the resulting mixture was stirred at r.t. overnight. DMF was removed under reduced pressure. The resulting slurry was purified by flash column chromatography (DCM/MeOH/NH₃ 8:2:0.2) affording the product as a thick yellow oil (1.12 g, 4.3 mmol, 58.6% yield).

Step B

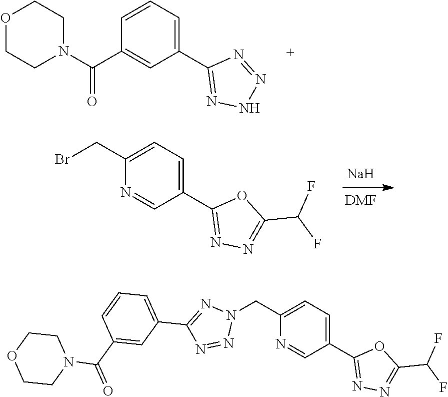

A mixture of morpholin-4-yl-[3-(2H-tetrazol-5-yl)phenyl]methanone (75 mg, 0.289 mmol, 1 equiv.) and sodium hydride (1.1 equiv.) in 1 mL of DMF was stirred at r.t. for 15 m in. 2-[6-(bromomethyl)pyridin-3-yl]-5-(difluoromethyl)-1,3,4-oxadiazole (Intermediate A, 83.9 mg, 1 equiv.) was added and the reaction mixture was stirred overnight. Full conversion was observed by LC-MS. The reaction mixture was diluted with water and precipitation occurred. The off-white solid was filtered, washed with water and dried. The crude product obtained (˜100 mg) was purified by prep-HPLC using neutral conditions. The product was further purified by p-TLC (DCM/MeOH 97:3) affording 12.5 mg (0.027 mmol, 9.22% yield) of pure product as a white solid. (m/z 469.00 [MH+]).

The following compounds were synthesized according to the same procedure:

| Compd. | Structure | m/z [MH+] |
|---|---|---|
| 29 | 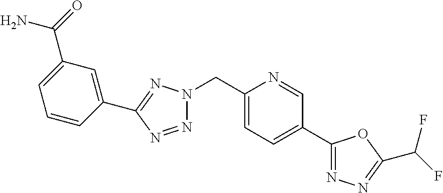 [SMILES: NC(=O)c1cccc(-c2nnn(Cc3ccc(-c4nnc(C(F)F)o4)cn3)n2)c1] | 440.07 |
| 50 | 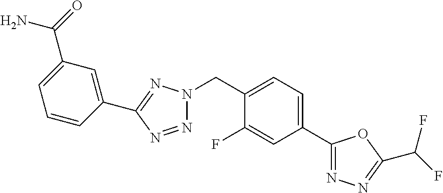 [SMILES: NC(=O)c1cccc(-c2nnn(Cc3ccc(-c4nnc(C(F)F)o4)cc3F)n2)c1] | 416.04 |
| 54 | 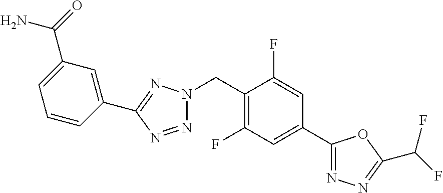 [SMILES: NC(=O)c1cccc(-c2nnn(Cc3c(F)cc(-c4nnc(C(F)F)o4)cc3F)n2)c1] | 434.08 |
| 80 | 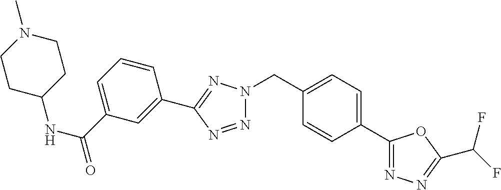 [SMILES: CN1CCC(NC(=O)c2cccc(-c3nnn(Cc4ccc(-c5nnc(C(F)F)o5)cc4)n3)c2)CC1] | 495.05 |
| 81 | 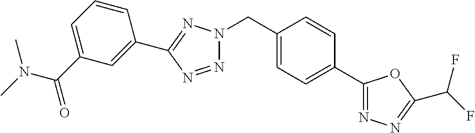 [SMILES: CN(C)C(=O)c1cccc(-c2nnn(Cc3ccc(-c4nnc(C(F)F)o4)cc3)n2)c1] | 426.1 |
| 89 | 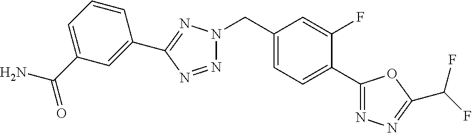 [SMILES: NC(=O)c1cccc(-c2nnn(Cc3ccc(-c4nnc(C(F)F)o4)c(F)c3)n2)c1] | 416.15 |
| 93 | 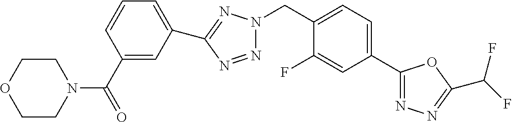 [SMILES: O=C(c1cccc(-c2nnn(Cc3ccc(-c4nnc(C(F)F)o4)cc3F)n2)c1)N1CCOCC1] | 486.12 |
| 112 | 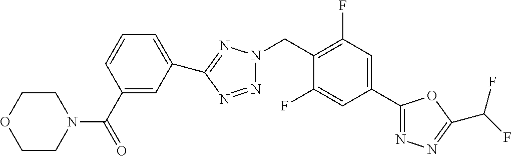 [SMILES: O=C(c1cccc(-c2nnn(Cc3c(F)cc(-c4nnc(C(F)F)o4)cc3F)n2)c1)N1CCOCC1] | 504.12 |
| 137 | 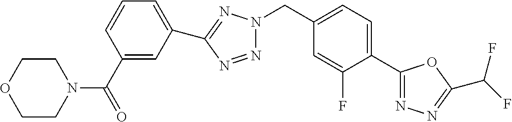 [SMILES: O=C(c1cccc(-c2nnn(Cc3ccc(-c4nnc(C(F)F)o4)c(F)c3)n2)c1)N1CCOCC1] | 486.12 |

### Example 10. Synthesis of 3-(2-(4-(5-(difluoromethyl)-1,3,4-oxadiazol-2-yl)benzyl)-2H-tetrazol-5-yl)benzamide. (Compd. 75)

Step A

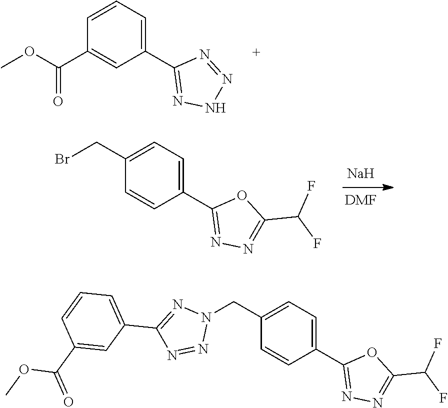

A mixture of methyl 3-(1H-tetrazol-5-yl)benzoate (995 mg, 4.87 mmol, 1 equiv.) and sodium hydride (1.1 equiv.) in 6 mL of DMF was stirred at r.t. for 15 min. 2-[4-(bromomethyl)phenyl]-5-(difluoromethyl)-1,3,4-oxadiazole (Intermediate B, 1.4 g, 1 equiv.) was added and the reaction mixture was stirred at r.t. for 4 h. Full conversion was observed by LC-MS. The reaction mixture was diluted with water and precipitation occurred. The white solid which formed was filtered and washed with water. Then it was dissolved in EtOAc and washed with brine. The organic layers were dried over MgSO₄, filtered and concentrated under reduced pressure to afford a white solid (1.7 g), which was used for the next step without any further purification.

Step B

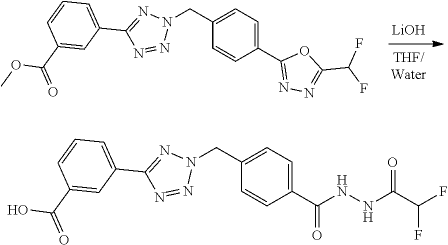

Methyl 3-[2-[[4-[5-(difluoromethyl)-1,3,4-oxadiazol-2-yl]phenyl]methyl]tetrazol-5-yl]benzoate (1.7 g, 4.12 mmol, 1 equiv.) was dissolved in 30 mL of a 1:1 THF/water mixture and lithium hydroxide monohydrate was added. The reaction mixture was stirred at 50° C. for 3 h. Full conversion was observed by LC-MS. THF was removed under reduced pressure, more water was added. The aqueous solution was acidified with 1M HCl and precipitation occurred. The white precipitate was filtered, washed with water and dried. The product (1.3 g) was used in the next step without further purification.

Step C

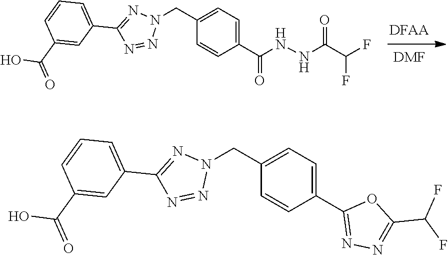

3-[2-[[4-[[(2,2-difluoroacetyl)amino]carbamoyl]phenyl]methyl]tetrazol-5-yl]benzoic acid (1.34 g, 1 equiv.) was dissolved in dry DMF (10 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at 70° C. for three hours. Full conversion was observed by LC-MS.

Water was added to the reaction mixture and precipitation occurred. The solid was filtered, washed with water and dried. The product was used in the next step without further purification.

Step D

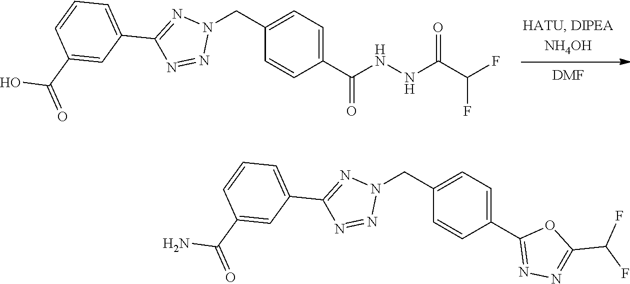

A mixture of 3-[2-[[4-[5-(difluoromethyl)-1,3,4-oxadiazol-2-yl]phenyl]methyl]tetrazol-5-yl]benzoic acid (100 mg, 0.25 mmol, 1 equiv.), HATU (2 equiv.) and DIPEA (3 equiv.) in 2.5 mL of DMF was stirred at r.t. for 30 min. A yellow clear solution was obtained. A solution of 25% aqueous ammonia (10 equiv.) was added and the resulting mixture was stirred at r.t. overnight. The reaction mixture was diluted with water and extracted with EtOAc. The combined organic layers were washed with brine, dried over MgSO₄, filtered and concentrated under reduced pressure. The resulting brown oil was purified by prep-HPLC affording the product as a white solid (15.1 mg, 0.036 mmol, 14.2% yield, m/z 397.95 [MH+]).

The following compounds were synthesized according to the same procedure:

| Compd. | Structure | m/z [MH+] |
|---|---|---|
| 260 | 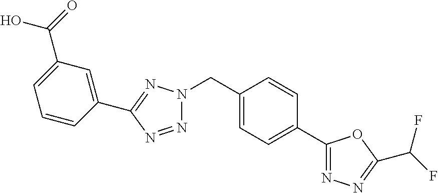 [SMILES: O=C(O)c1cccc(-c2nnn(Cc3ccc(-c4nnc(C(F)F)o4)cc3)n2)c1] | 389.1 |
| 232 | 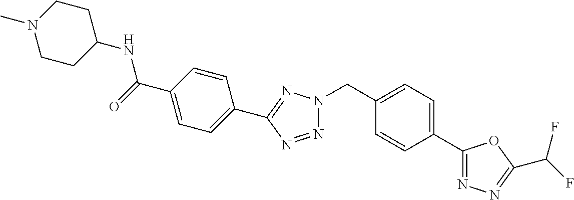 [SMILES: CN1CCC(NC(=O)c2ccc(-c3nnn(Cc4ccc(-c5nnc(C(F)F)o5)cc4)n3)cc2)CC1] | 495.14 |
| 96 | 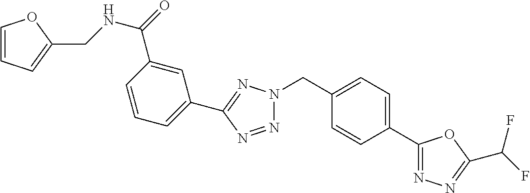 [SMILES: O=C(NCc1ccco1)c1cccc(-c2nnn(Cc3ccc(-c4nnc(C(F)F)o4)cc3)n2)c1] | 478.0 |
| 120 | 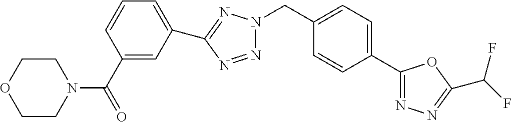 [SMILES: O=C(c1cccc(-c2nnn(Cc3ccc(-c4nnc(C(F)F)o4)cc3)n2)c1)N1CCOCC1] | 468.0 |
| 230 | 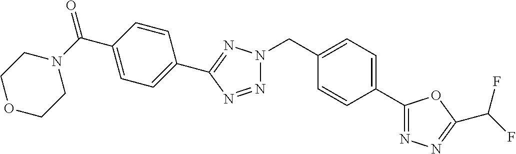 [SMILES: O=C(c1ccc(-c2nnn(Cc3ccc(-c4nnc(C(F)F)o4)cc3)n2)cc1)N1CCOCC1] | 468.0 |

### Example 11. Synthesis of 2-(difluoromethyl)-5-(4-((5-(3-(4,5,6,7-tetrahydropyrazolo[1,5-a]pyrazin-3-yl)phenyl)-2H-tetrazol-2-yl)methyl)phenyl)-1,3,4-oxadiazole. (Compd. 111)

Step A

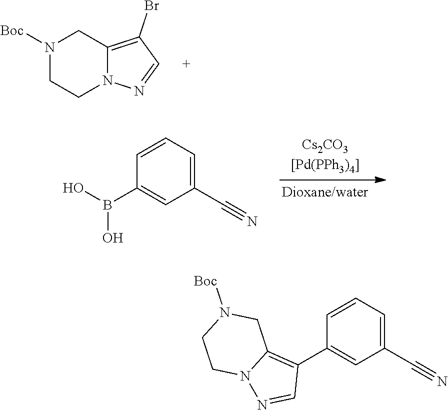 [SMILES: CC(C)(C)OC(=O)N1CCn2ncc(Br)c2C1 ; N#Cc1cccc(B(O)O)c1 ; CC(C)(C)OC(=O)N1CCn2ncc(-c3cccc(C#N)c3)c2C1]

Tetrakis(triphenylphosphine)palladium(0) (76.48 mg, 0.066 mmol, 0.08 equiv.) was added to a suspension of tert-butyl-3-bromo-6,7-dihydro-4H-pyrazolo[1,5-a]pyrazine-5-carboxylate (250 mg, 0.827 mmol, 1 equiv.), (3-cyanophenyl)boronic acid (145.88 mg, 0.99 mmol, 1.2 equiv.) and cesium carbonate (808.7 mg, 2.48 mmol, 3 equiv.) in 9 mL 1:2 water/dioxane. The reaction mixture was degassed and stirred at 80° C. for 2 hours. Then it was diluted with EtOAc and filtrated over Celite®. The organic phase was washed with water (twice), dried over Na₂SO₄, filtered and concentrated under reduced pressure. Crude was used for the next step without any purification.

Step B

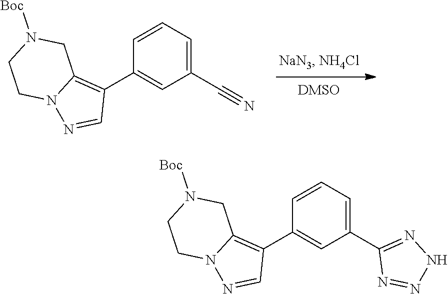 [SMILES: CC(C)(C)OC(=O)N1CCn2ncc(-c3cccc(C#N)c3)c2C1 ; CC(C)(C)OC(=O)N1CCn2ncc(-c3cccc(-c4nn[nH]n4)c3)c2C1]

Sodium azide (2.5 equiv.) and ammonium acetate (2.5 equiv.) were added to a solution of tert-butyl 3-(3-cyanophenyl)-6,7-dihydropyrazolo[1,5-a]pyrazine-5 (4H)-carboxylate in DMSO (5 mL). The reaction mixture was stirred at 80° C. for 48 h, then it was diluted with water and ethyl acetate. The two phases were separated and the aqueous phase was acidified with 1M HCl (pH=4) and extracted with EtOAc. The organic phase was dried over Na₂SO₄, filtered and concentrated under reduced pressure. Product was used for the next step without any purification.

Step C

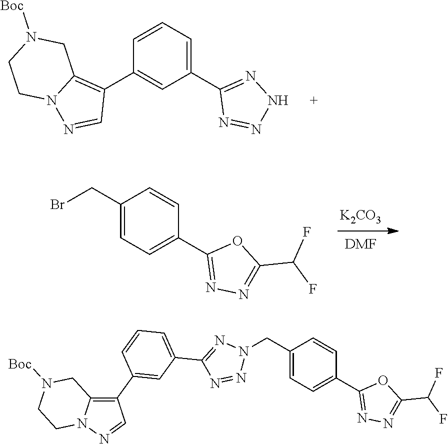

Potassium carbonate (78 mg, 0.562 mmol, 2 equiv.) was added to a solution of tert-butyl 3-[3-(2H-tetrazol-5-yl)phenyl]-6,7-dihydro-4H-pyrazolo[1,5-a]pyrazine-5-carboxylate (129 mg, 0.28 mmol, 1 equiv.) and 2-[4-(bromomethyl)phenyl]-5-(difluoromethyl)-1,3,4-oxadiazole (Intermediate B, 89 mg, 0.309 mmol, 1.1 equiv.) in 1 mL DMF, and the resulting mixture was stirred at r.t. overnight. Full conversion was verified by LC-MS. Reaction mixture was diluted with water and precipitation occurred. The solid was filtered and used for the next step without any purification.

Step D

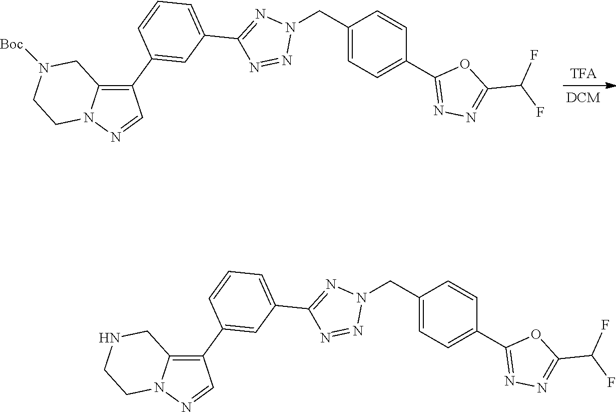 [SMILES: CC(C)(C)OC(=O)N1CCn2ncc(-c3cccc(-c4nnn(Cc5ccc(-c6nnc(C(F)F)o6)cc5)n4)c3)c2C1 ; FC(F)c1nnc(-c2ccc(Cn3nnc(-c4cccc(-c5cnn6c5CNCC6)c4)n3)cc2)o1]

Trifluoroacetic acid (0.119 mL, 15 equiv.) was added to a solution of tert-butyl 3-[3-[2-[[4-[5-(difluoromethyl)-1,3,4-oxadiazol-2-yl]phenyl]methyl]tetrazol-5-yl]phenyl]-6,7-dihydro-4H-pyrazolo[1,5-a]pyrazine-5-carboxylate (70 mg, 0.103 mmol, 1 equiv.) in dichloromethane (1 mL) and the reaction mixture was stirred at r.t. for 2 h. The progress of the reaction was monitored by LC-MS. The reaction mixture was diluted with extra DCM and washed with NaHCO₃ (3 times). Organic phase was dried over Na₂SO₄, filtered and dried under reduced pressure. Purification of the crude by prep-HPLC in neutral condition afforded 4.1 mg (0.008 mmol, 8.2% yield) of pure product (m/z 475.97 [MH+]).

The following compounds were synthesized according to the same procedure:

| Compd. | Structure | m/z [MH+] |
|---|---|---|
| 108 | 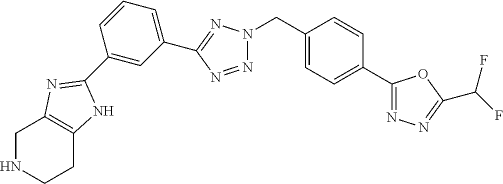 [SMILES: FC(F)c1nnc(-c2ccc(Cn3nnc(-c4cccc(-c5nc6c([nH]5)CCNC6)c4)n3)cc2)o1] | 475.97 |
| 240 | 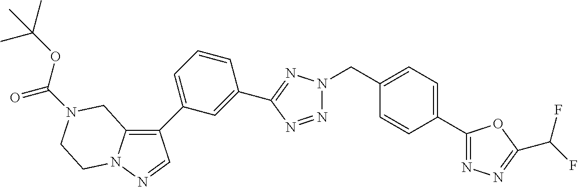 [SMILES: CC(C)(C)OC(=O)N1CCn2ncc(-c3cccc(-c4nnn(Cc5ccc(-c6nnc(C(F)F)o6)cc5)n4)c3)c2C1] | 576.02 |
| 272 | 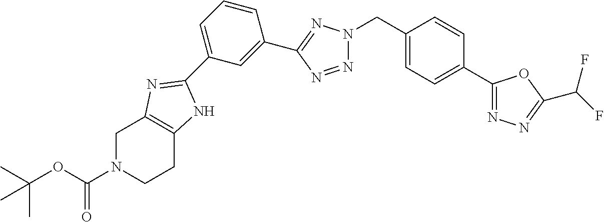 [SMILES: CC(C)(C)OC(=O)N1CCc2[nH]c(-c3cccc(-c4nnn(Cc5ccc(-c6nnc(C(F)F)o6)cc5)n4)c3)nc2C1] | 576.28 |
| 276 | 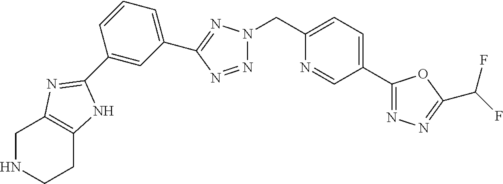 [SMILES: FC(F)c1nnc(-c2ccc(Cn3nnc(-c4cccc(-c5nc6c([nH]5)CCNC6)c4)n3)nc2)o1] | 477.18 |
| 353 | 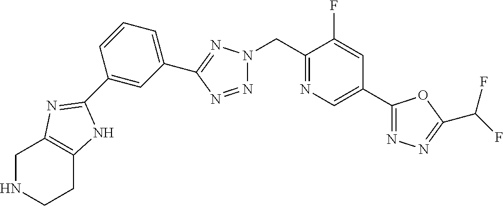 [SMILES: Fc1cc(-c2nnc(C(F)F)o2)cnc1Cn1nnc(-c2cccc(-c3nc4c([nH]3)CCNC4)c2)n1] | 495.33 |
| 354 | 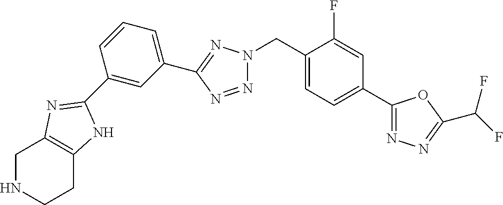 [SMILES: Fc1cc(-c2nnc(C(F)F)o2)ccc1Cn1nnc(-c2cccc(-c3nc4c([nH]3)CCNC4)c2)n1] | 494.24 |
| 355 | 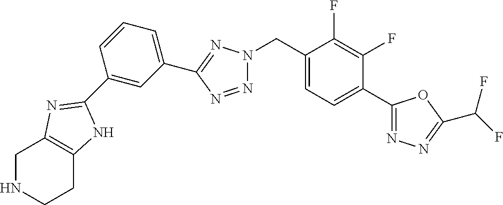 [SMILES: Fc1c(Cn2nnc(-c3cccc(-c4nc5c([nH]4)CCNC5)c3)n2)ccc(-c2nnc(C(F)F)o2)c1F] | 511.97 |
| 364 | 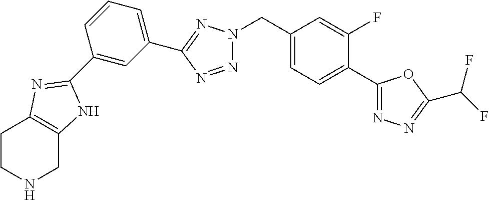 [SMILES: Fc1cc(Cn2nnc(-c3cccc(-c4nc5c([nH]4)CNCC5)c3)n2)ccc1-c1nnc(C(F)F)o1] | 494.13 |
| 365 | 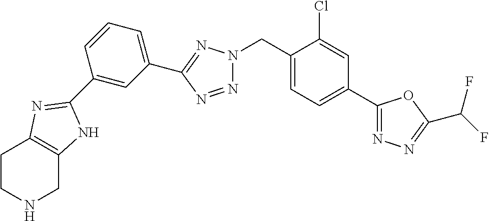 [SMILES: FC(F)c1nnc(-c2ccc(Cn3nnc(-c4cccc(-c5nc6c([nH]5)CNCC6)c4)n3)c(Cl)c2)o1] | 510.08 |
| 367 | 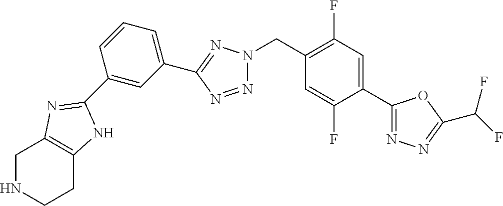 [SMILES: Fc1cc(-c2nnc(C(F)F)o2)c(F)cc1Cn1nnc(-c2cccc(-c3nc4c([nH]3)CCNC4)c2)n1] | 512.16 |

### Example 12. Synthesis of 2-(difluoromethyl)-5-(4-((5-(6-(piperazin-1-yl)pyridin-3-yl)-2H-tetrazol-2-yl)methyl)phenyl)-1,3,4-oxadiazole. (Compd. 148)

Step A

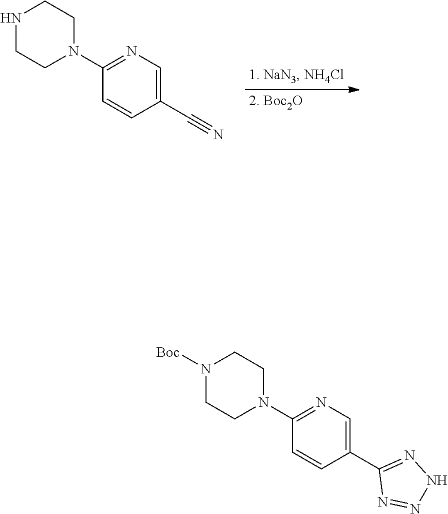 [SMILES: N#Cc1ccc(N2CCNCC2)nc1 ; CC(C)(C)OC(=O)N1CCN(c2ccc(-c3nn[nH]n3)cn2)CC1]

6-Piperazin-1-ylpyridine-3-carbonitrile (600 mg, 3.18 mmol, 1 equiv.), sodium azide (455.9 mg, 7.01 mmol, 2.2 equiv.) and ammonium chloride (375.11 mg, 7.01 mmol, 2.2 equiv.) were suspended in DMSO (6 mL) and the reaction mixture was stirred at 80° C. overnight. The reaction mixture was cooled down to r.t. and di-tert-butyl dicarbonate (1391.4 mg, 6.37 mmol, 2 equiv.) was added. After stirring overnight, the reaction mixture was diluted with water and acidified with acetic acid (pH=3). The product precipitated as white solid, which was collected by filtration, washed with water and used for the next step without any further purification (980 mg, 2.8 mmol, 88% yield).

Step B

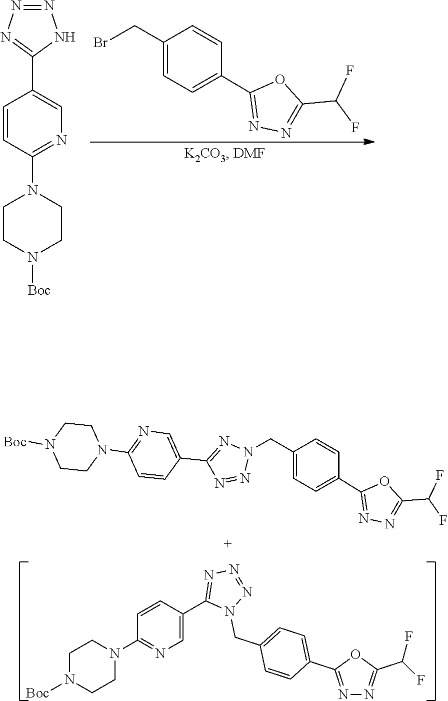

Potassium carbonate (79.2 mg, 0.57 mmol, 2 equiv.) was added to a solution of tert-butyl 4-[5-(2H-tetrazol-5-yl)pyridin-2-yl]piperazine-1-carboxylate (100 mg, 0.29 mmol, 1 equiv.) and 2-[4-(bromomethyl)phenyl]-5-(difluoromethyl)-1,3,4-oxadiazole (Intermediate B, 83 mg, 0.29 mmol, 1 equiv.) in 2 mL DMF. The resulting mixture was stirred at r.t. overnight. The mixture was then diluted with water. The precipitate which formed was recovered by filtration, dried and used for the next step without any purification.

Step C

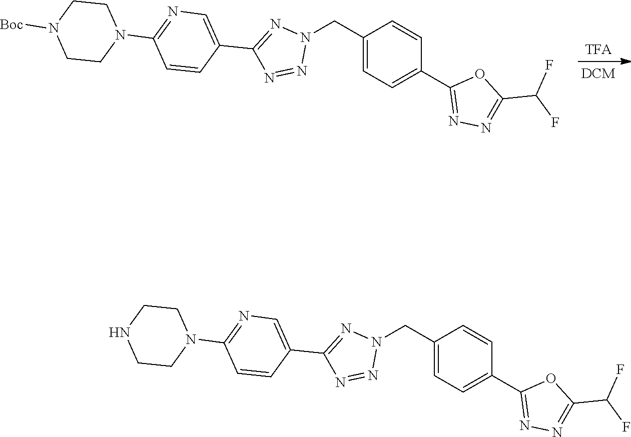 [SMILES: CC(C)(C)OC(=O)N1CCN(c2ccc(-c3nnn(Cc4ccc(-c5nnc(C(F)F)o5)cc4)n3)cn2)CC1 ; FC(F)c1nnc(-c2ccc(Cn3nnc(-c4ccc(N5CCNCC5)nc4)n3)cc2)o1]

tert-butyl-4-(5-(2-(4-(5-(difluoromethyl)-1,3,4-oxadiazol-2-yl)benzyl)-2H-tetrazol-5-yl)pyridin-2-yl)piperazine-1-carboxylate was suspended in DCM and TFA (10 equiv.) was added. The reaction mixture was stirred at r.t. for 2 h. Full conversion was observed by LC-MS. Reaction mixture was diluted with EtOAc and washed two times with a solution of sodium bicarbonate and brine. Organic phase was dried over Na₂SO₄, filtered and evaporated to afford a crude product, which was purified by prep-HPLC in neutral conditions. 24 mg (0.054 mmol, 19% yield) of pure product were obtained (m/z 440.05 [MH+]).

The following compounds were synthesized according to the same procedure:

| Compd. | Structure | m/z [MH+] |
|---|---|---|
| 33 | 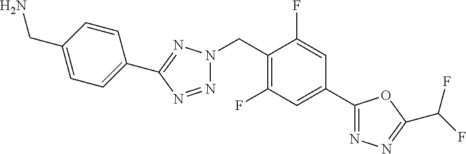 [SMILES: NCc1ccc(-c2nnn(Cc3c(F)cc(-c4nnc(C(F)F)o4)cc3F)n2)cc1] | 461.14 |
| 43 | 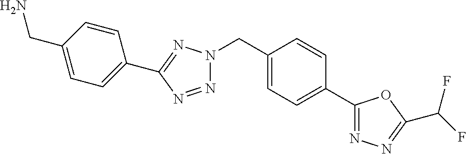 [SMILES: NCc1ccc(-c2nnn(Cc3ccc(-c4nnc(C(F)F)o4)cc3)n2)cc1] | 384.25 |
| 103 | 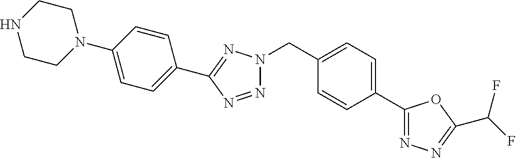 [SMILES: FC(F)c1nnc(-c2ccc(Cn3nnc(-c4ccc(N5CCNCC5)cc4)n3)cc2)o1] | 439.21 |
| 159 | 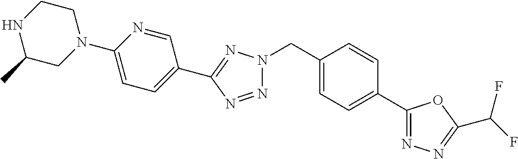 [SMILES: C[C@@H]1CN(c2ccc(-c3nnn(Cc4ccc(-c5nnc(C(F)F)o5)cc4)n3)cn2)CCN1] | 454.05 |
| 182 | 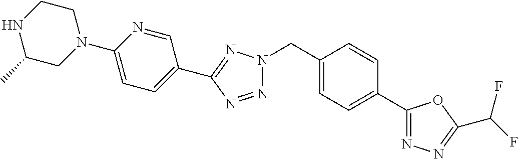 [SMILES: C[C@H]1CN(c2ccc(-c3nnn(Cc4ccc(-c5nnc(C(F)F)o5)cc4)n3)cn2)CCN1] | 454.04 |
| 224 | 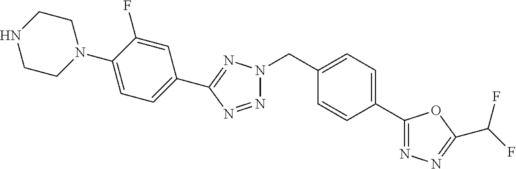 [SMILES: Fc1cc(-c2nnn(Cc3ccc(-c4nnc(C(F)F)o4)cc3)n2)ccc1N1CCNCC1] | 457.04 |

*[M + ACN + H]⁺ was observed.

### Example 13. Synthesis of N-(5-(2-(4-(5-(difluoromethyl)-1,3,4-oxadiazol-2-yl)benzyl)-2H-tetrazol-5-yl)-2-(methylamino)phenyl)morpholine-4-carboxamide (Compd. 176) and of 4-(2-(4-(5-(difluoromethyl)-1,3,4-oxadiazol-2-yl)benzyl)-2H-tetrazol-5-yl)-N-1-methylbenzene-1,2-diamine (Compd. 164)

Step A

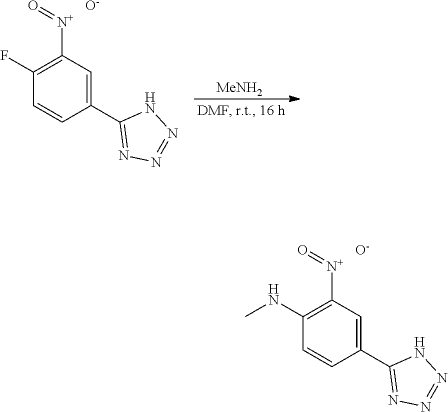

5-(4-fluoro-3-nitrophenyl)-2H-tetrazole (1 g, 4.78 mmol, 1 equiv.) was dissolved in DMF (10 mL). A solution of methylamine 2M in THF was added (10 equiv.) and the reaction mixture was stirred at r.t. overnight. Full conversion was confirmed by LC-MS. Reaction mixture was evaporated under reduced pressure and the crude was used for the next step without any further purification.

Step B

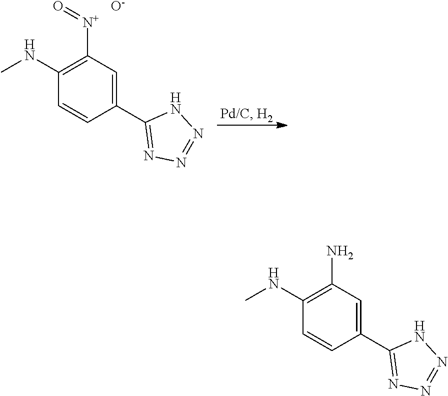

Palladium on activated carbon (0.2 equiv.) was added to a solution of N-methyl-2-nitro-4-(2H-tetrazol-5-yl)aniline (1 g, 4.5 mmol, 1 equiv.) in MeOH (150 mL) under inert gas. The flask was then filled with H₂ and the reaction mixture was stirred at r.t overnight. Precipitation occurred. The solid (300 mg, 1.57 mmo, 34.7% yield) was filtered over sintered glass and used for the next step.

Step C

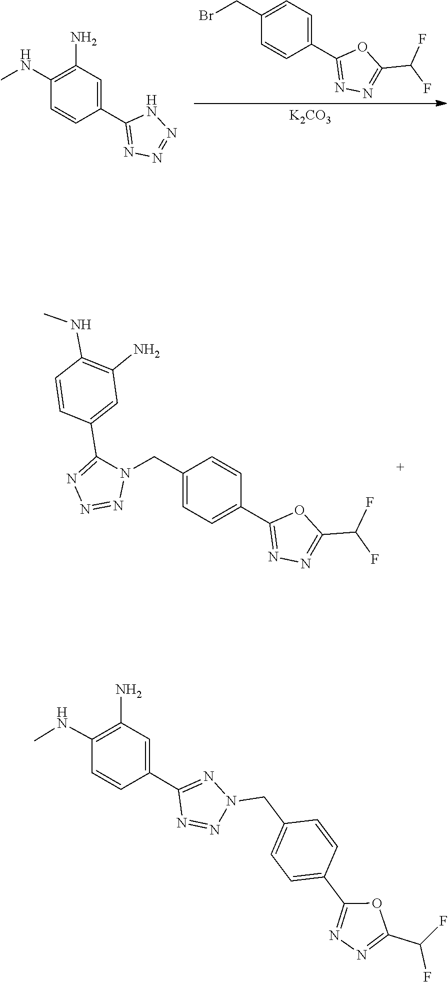 [SMILES: CNc1ccc(-c2nnn[nH]2)cc1N ; FC(F)c1nnc(-c2ccc(CBr)cc2)o1 ; "K2CO3" ; CNc1ccc(-c2nnnn2Cc2ccc(-c3nnc(C(F)F)o3)cc2)cc1N ; CNc1ccc(-c2nnn(Cc3ccc(-c4nnc(C(F)F)o4)cc3)n2)cc1N]

1-N-methyl-4-(2H-tetrazol-5-yl)benzene-1,2-diamine (300 mg, 1.57 mmol, 1 equiv.) was suspended in DMF (3 mL). 2-[4-(bromomethyl)phenyl]-5-(difluoromethyl)-1,3,4-oxadiazole (Intermediate B, 300.92 mg, 1.041, 0.66 equiv.) and potassium carbonate (326.98 mg, 2.36 mmol, 1.5 equiv.) were added and the reaction mixture was stirred at r.t. overnight. Full conversion was observed by LC-MS. The reaction mixture was diluted with water and the product was extracted with EtOAc. The organic phase was dried over Na₂SO₄, filtered and evaporated under reduced pressure to afford crude product, which was purified by prep-HPLC. 60 mg of pure product (0.15 mmol, 9.5% yield) were obtained (Compd. 164, m/z 399.01 [MH+]). 16 mg of 4-[1-[[4-[5-(difluoromethyl)-1,3,4-oxadiazol-2-yl]phenyl]methyl]tetrazol-5-yl]-1-N-methylbenzene-1,2-diamine (0.04 mmol) were also recovered (m/z 399.01 [MH+]).

Step D

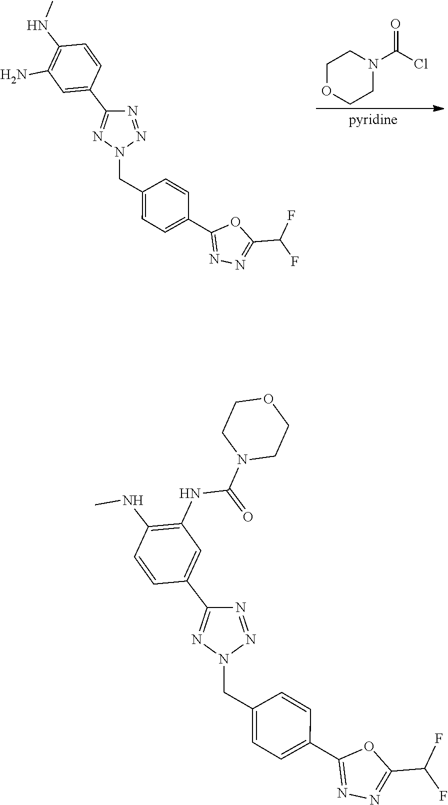 [SMILES: CNc1ccc(-c2nnn(Cc3ccc(-c4nnc(C(F)F)o4)cc3)n2)cc1N ; O=C(Cl)N1CCOCC1 ; CNc1ccc(-c2nnn(Cc3ccc(-c4nnc(C(F)F)o4)cc3)n2)cc1NC(=O)N1CCOCC1]

Morpholine-4-carbonyl chloride (12.4 mg, 0.083 mmol, 1.1 equiv.) was added to a solution of 4-[2-[[4-[5-(difluoromethyl)-1,3,4-oxadiazol-2-yl]phenyl]methyl]tetrazol-5-yl]-1-N-methylbenzene-1,2-diamine (30 mg, 0.075 mmol, 1 equiv.) in pyridine (2 mL). The reaction mixture was stirred at 40° C. for 1 h. Full conversion was observed by LC-MS. Solvent was evaporated under reduced pressure and the crude was purified by prep-HPLC. 16.6 mg (0.032 mmol, 42.9% yield) of pure product were obtained (compd. 176, m/z 512.05 [MH+]).

### Example 14. Synthesis of 4-(2-(4-(5-(difluoromethyl)-1,3,4-oxadiazol-2-yl)-2,6-difluorobenzyl)-2H-tetrazol-5-yl)-2-(morpholine-4-carboxamido)phenyl morpholine-4-carboxylate (Compd. 196,) and of 2-amino-4-(2-(4-(5-(difluoromethyl)-1,3,4-oxadiazol-2-yl)-2,6-difluorobenzyl)-2H-tetrazol-5-yl)phenyl morpholine-4-carboxylate (Compd. 160)

Step A

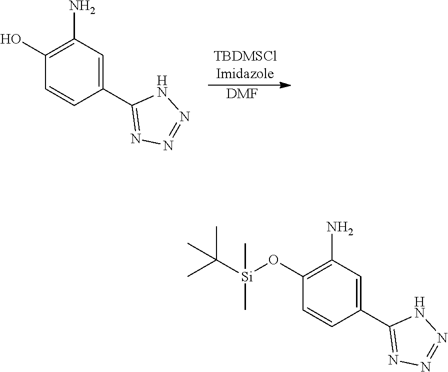

A solution of 2-amino-4-(2H-tetrazol-5-yl)phenol (500 mg, 2.82 mmol, 1 equiv.), tert-butylchlorodimethylsilane (680.61 mg, 4.5 mmol, 1.6 equiv.) and imidazole (345.86 mg, 5.08 mmol, 1.8 equiv.) in DMF (4 mL) was stirred at r.t. overnight. Full conversion was observed by LC-MS. The reaction mixture was diluted with water and precipitation occurred. The solid product (690 mg, 2.37 mmol, 83.9% yield) was filtered, washed with n-hexane, dried and used without any purification for the next step.

Step B

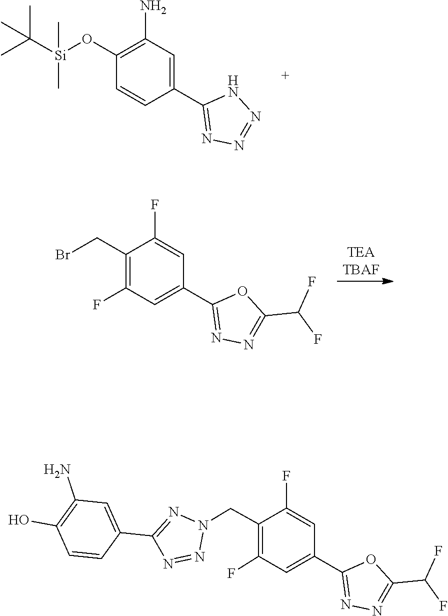

2-[4-(bromomethyl)-3,5-difluorophenyl]-5-(difluoromethyl)-1,3,4-oxadiazole (Intermediate C, 123 mg, 0.377 mmol, 1.1 equiv.) was added to a solution of 2-[tert-butyl(dimethyl)silyl]oxy-5-(2H-tetrazol-5-yl)aniline (100 mg, 0.343 mmol, 1 equiv.) and triethylamine (0.096 mL, 0.686 mmol, 2 equiv.) in acetonitrile (3 mL). The resulting mixture was stirred at r.t. for 4 days. Full conversion and partial hydroxy deprotection were observed by LC-MS. Tetrabutylammonium fluoride (54 mg, 0.206 mmol, 0.6 equiv.) was added to the reaction mixture. Full deprotection was observed. Solvent was evaporated under reduced pressure and crude was purified by prep-HPLC. 54 mg of product (0.103 mmol, 29.9% yield) was obtained.

Step C

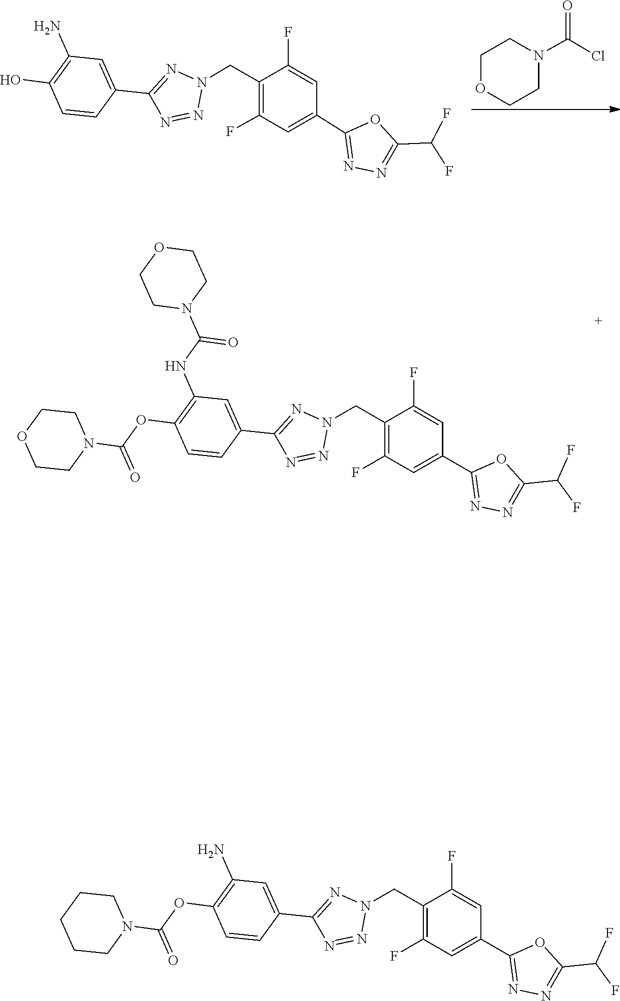

Morpholine-4-carbonyl chloride (23 mg, 0.154 mmol, 1.2 equiv.) was added dropwise to a solution of 2-amino-4-[2-[[4-[5-(difluoromethyl)-1,3,4-oxadiazol-2-yl]-2,6-difluorophenyl]methyl]tetrazol-5-yl]phenol (54 mg, 0,128 mmol, 1 equiv.) in pyridine (2 mL). The reaction mixture was stirred at r.t. overnight. Full conversion of the starting material was observed by LC-MS. Solvent was evaporated under reduced pressure and crude was purified by prep-HPLC. 6 mg of [4-[2-[[4-[5-(difluoromethyl)-1,3,4-oxadiazol-2-yl]-2,6-difluorophenyl]methyl]tetrazol-5-yl]-2-(morpholine-4-carbonylamino)phenyl]-morpholine-4-carboxylate (compd. 196, m/z 535.0 [MH+]) and 9.7 mg of [2-amino-4-[2-[[4-[5-(difluoromethyl)-1,3,4-oxadiazol-2-yl]-2,6-difluorophenyl]methyl]tetrazol-5-yl]phenyl]-morpholine-4-carboxylate (compd. 160, m/z 647.99 [MH+]) were obtained.

The following compounds were synthesized according to the same procedure:

| Compd. | Structure | m/z [MH+] |
|---|---|---|
| 2 | 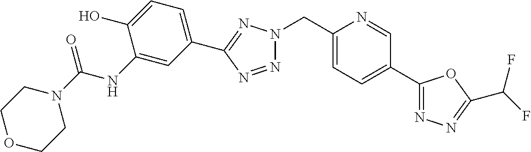 [SMILES: O=C(Nc1cc(-c2nnn(Cc3ccc(-c4nnc(C(F)F)o4)cn3)n2)ccc1O)N1CCOCC1] | 500.06 |
| 25 | 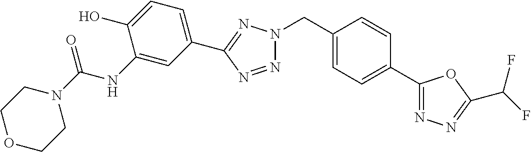 [SMILES: O=C(Nc1cc(-c2nnn(Cc3ccc(-c4nnc(C(F)F)o4)cc3)n2)ccc1O)N1CCOCC1] | 499.0 |
| 31 | 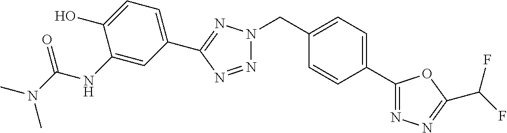 [SMILES: CN(C)C(=O)Nc1cc(-c2nnn(Cc3ccc(-c4nnc(C(F)F)o4)cc3)n2)ccc1O] | 457.17 |
| 71 | 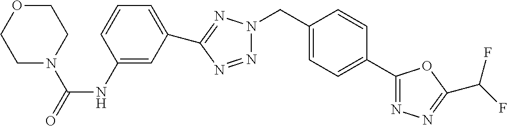 [SMILES: O=C(Nc1cccc(-c2nnn(Cc3ccc(-c4nnc(C(F)F)o4)cc3)n2)c1)N1CCOCC1] | 483.05 |
| 95 | 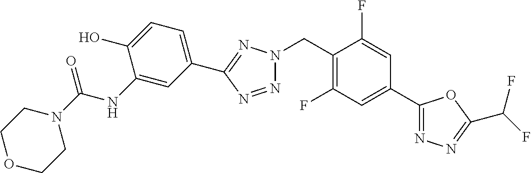 [SMILES: O=C(Nc1cc(-c2nnn(Cc3c(F)cc(-c4nnc(C(F)F)o4)cc3F)n2)ccc1O)N1CCOCC1] | 534.97 |
| 98 | 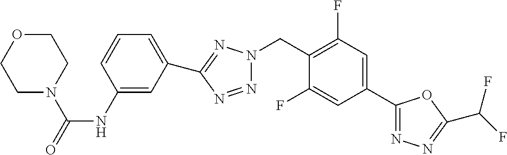 [SMILES: O=C(Nc1cccc(-c2nnn(Cc3c(F)cc(-c4nnc(C(F)F)o4)cc3F)n2)c1)N1CCOCC1] | 519.13 |
| 258 | 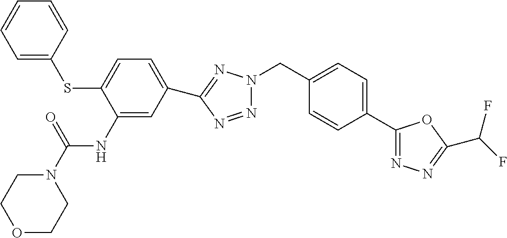 [SMILES: O=C(Nc1cc(-c2nnn(Cc3ccc(-c4nnc(C(F)F)o4)cc3)n2)ccc1Sc1ccccc1)N1CCOCC1] | 591.07 |

### Example 15. Synthesis of N-(5-(2-(4-(5-(difluoromethyl)-1,3,4-oxadiazol-2-yl)benzyl)-2H-tetrazol-5-yl)pyridin-3-yl)morpholine-4-carboxamide (Compd. 199)

Step A

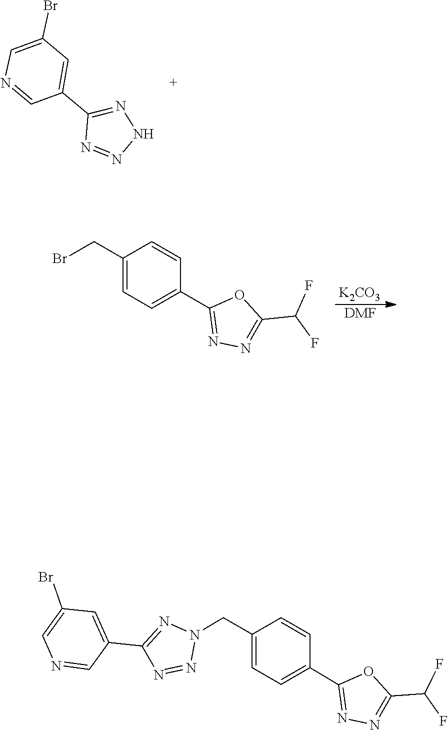

3-bromo-5-(2H-tetrazol-5-yl)pyridine (200 mg, 0.885 mmol, 1 equiv.) and potassium carbonate (244.59 mg, 1.77 mmol, 2 equiv.) were suspended in DMF (3 mL). After 15 min 2-[4-(bromomethyl)phenyl]-5-(difluoromethyl)-1,3,4-oxadiazole (Intermediate B, 281.37 mg, 0.973 mmol, 1.1 equiv.) was added to the suspension and the reaction mixture was stirred at r.t. overnight. Full conversion was observed by LC-MS. Water was added to the reaction mixture and precipitation occurred. Solid was filtered and purified by prep-HPLC, affording pure product.

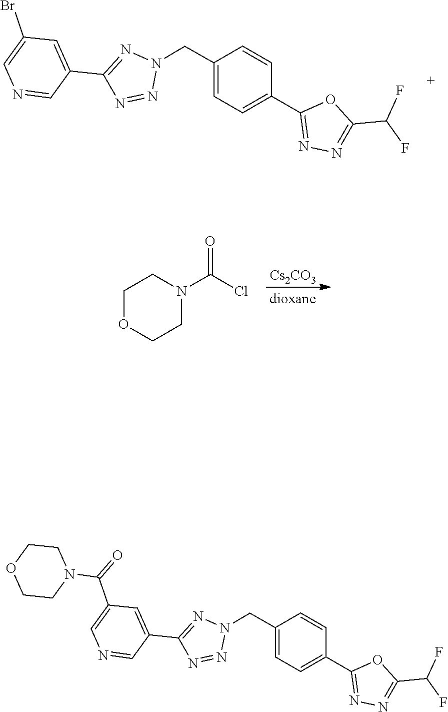

Tris(dibenzylideneacetone)dipalladium(0) (23.73 mg, 0.026 mmol, 0.1 equiv.) and Xantphos (29.95 mg, 0.052 mmol, 0.2 equiv.) were added to a solution of 2-[4-[[5-(5-bromopyridin-3-yl)tetrazol-2-yl]methyl]phenyl]-5-(difluoromethyl)-1,3,4-oxadiazole (125 mg, 0.259 mmol, 1 equiv.), morpholine-4-carboxamide (67.44 mg, 0.518 mmol, 2 equiv.) and cesium carbonate (168.84 mg, 0.518 mmol, 2 equiv.) in degassed 1,4-dioxane (2 mL). The reaction mixture was degassed with Ar for 20 min and heated to 80° C. overnight. Reaction mixture was diluted with EtOAc and filtered on Celite®. Filtrate was washed twice with aqueous NaHCO₃ and brine, dried over Na₂SO₄, filtered and evaporated under reduced pressure. Crude was purified by prep-HPLC in neutral conditions. Pure product (m/z 484.05 [MH+]) was obtained (2.3 mg, 0.004 mmol, 1.65% yield).

### Example 16. Synthesis of 7′-(2-((5-(5-(difluoromethyl)-1,3,4-oxadiazol-2-yl)pyridin-2-yl)methyl)-2H-tetrazol-5-yl)-1′,4′-dihydro-3′H-spiro[cyclopentane-1,2′-quinoxalin]-3′-one (Compd. 27)

Step A

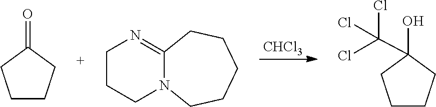

1,8-Diazabicyclo[5.4.0]undec-7-ene (8.9 mL, 60.03 mmol, 1 equiv.) was added dropwise to a mixture of cyclopentanone (5 g, 60.03 mmol, 1 equiv.) and dry chloroform (9.7 mL, 120 mmol, 2 equiv.) under an argon atmosphere. The reaction mixture was stirred at r.t. for 48 h, then diluted with dichloromethane (25 mL), washed with 1N HCl, water and brine, dried over Na₂SO₄, and concentrated under reduced pressure. The residual dark liquid was used in the next step without any purification.

Step B

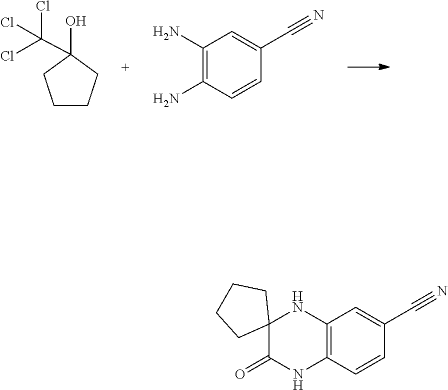 [SMILES: OC1(C(Cl)(Cl)Cl)CCCC1 ; N#Cc1ccc(N)c(N)c1 ; N#Cc1ccc2c(c1)NC1(CCCC1)C(=O)N2]

50% aqueous sodium hydroxide (1.4 mL) was added dropwise to a solution of 3,4-diaminobenzonitrile (700 mg, 5.26 mmol, 1 equiv.), 1-(trichloromethyl)cyclopentan-1-ol (2.1 g, 10.5 mmol, 2 equiv.) and benzyltriethylammonium chloride (120.28 mg, 0.52 mmol, 0.1 equiv.) in DCM (40 mL) at 0° C., under Ar. The reaction mixture was stirred at 0° C. for 1 h and then at r.t. overnight. The reaction mixture was diluted with water until complete dissolution. The layers were separated and the aqueous layer was extracted with DCM. The combined organic layers were dried over MgSO₄, filtered and concentrated under reduced pressure. The residue was purified by flash column chromatography (hexane/EtOAc 85:15 to 1:1) affording the desired product as a white solid (isomeric structure was confirmed by NOESY).

Step C

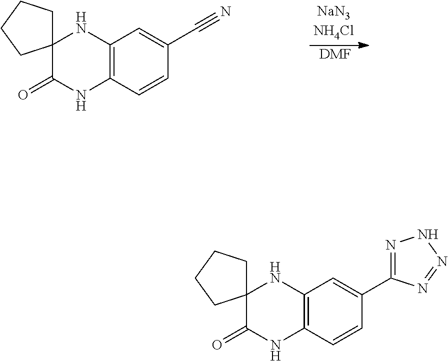

A mixture of 2-oxospiro[1,4-dihydroquinoxaline-3,1′-cyclopentane]-6-carbonitrile (240 mg, 1.06 mmol, 1 equiv.), sodium azide (137.3 mg, 2.11 mmol, 2 equiv.) and ammonium chloride (112.9 mg, 2.11 mmol, 2 equiv.) in DMF was stirred at 100° C. overnight. Water (15 mL) was added to the reaction mixture, followed by ethyl acetate (15 mL). The layers were separated. Acetic acid (300 μL, 4 equiv.) was added to the water phase and precipitation occurred after a few minutes. The white solid was filtered, washed with water and dried. The product was used in the next step without further purification.

Step D

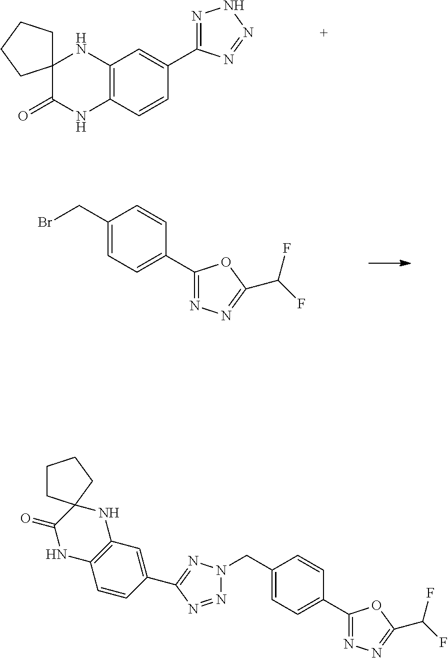 [SMILES: O=C1Nc2ccc(-c3nn[nH]n3)cc2NC12CCCC2 ; FC(F)c1nnc(-c2ccc(CBr)cc2)o1 ; O=C1Nc2ccc(-c3nnn(Cc4ccc(-c5nnc(C(F)F)o5)cc4)n3)cc2NC12CCCC2]

6-(2H-tetrazol-5-yl)spiro[1,4-dihydroquinoxaline-3,1′-cyclopentane]-2-one (120 mg, 0.444 mmol, 1 equiv.) and potassium carbonate (67.5 mg, 0.488 mmol, 1.1 equiv.) were suspended in DMF (3 mL). After 15 min 2-(4-(bromomethyl)phenyl)-5-(difluoromethyl)-1,3,4-oxadiazole (Intermediate B, 129 mg, 0.444 mmol, 1 equiv.) was added to the suspension and the reaction mixture was stirred at r.t. for 1 h. Full conversion was observed by LC-MS. Water was added to the reaction mixture and the product was extracted with ethyl acetate. The organic phase was washed several times with aqueous sodium bicarbonate and brine. After concentration under reduced pressure the residue (120 mg) was purified by prep-HPLC using neutral conditions. Pure product (m/z 480.12 [MH+]) was isolated as a white solid (26 mg, 0.054 mmol, 12% yield).

The following compounds were synthesized according to the same procedure:

| Compd. | Structure | m/z [MH+] |
|---|---|---|
| 110 | 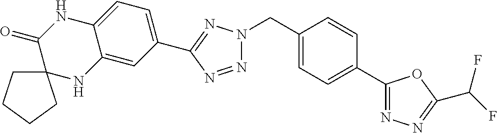 [SMILES: O=C1Nc2ccc(-c3nnn(Cc4ccc(-c5nnc(C(F)F)o5)cc4)n3)cc2NC12CCCC2] | 478.9 |
| 157 | 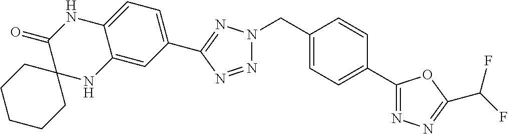 [SMILES: O=C1Nc2ccc(-c3nnn(Cc4ccc(-c5nnc(C(F)F)o5)cc4)n3)cc2NC12CCCCC2] | 492.18 |
| 195 | 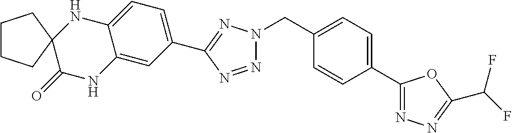 [SMILES: O=C1Nc2cc(-c3nnn(Cc4ccc(-c5nnc(C(F)F)o5)cc4)n3)ccc2NC12CCCC2] | 478.97 |
| 209 | 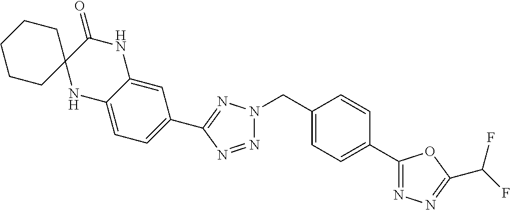 [SMILES: O=C1Nc2cc(-c3nnn(Cc4ccc(-c5nnc(C(F)F)o5)cc4)n3)ccc2NC12CCCCC2] | 493.17 |

### Example 17. Synthesis of 7-(2-(4-(5-(difluoromethyl)-1,3,4-oxadiazol-2-yl)benzyl)-2H-tetrazol-5-yl)-2-methyl-3,4-dihydroisoquinolin-1 (2H)-one (Compd. 173)

Step A

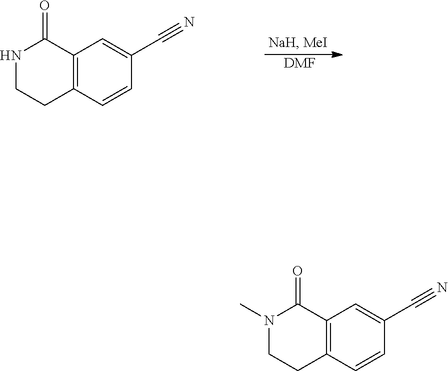

Sodium hydride (69.69 mg, 1.74 mmol, 1.2 equiv.) was added to a solution of 1-oxo-3,4-dihydro-2H-isoquinoline-7-carbonitrile (250 mg, 1.45 mmol, 1 equiv.) in DMF (10 mL). After 15 min methyl iodide (0.18 mL, 2.9 mmol, 2 equiv.) was added to the suspension and the dark brown reaction mixture was stirred at r.t. for 5 h. Water was added to the reaction mixture and the product was extracted with ethyl acetate. The aqueous layer was basified (K₂CO₃) and extracted with ethyl acetate. The combined organic layers were washed with brine, dried over MgSO₄, filtered and concentrated under reduced pressure. The product was used directly in the next step.

Step B

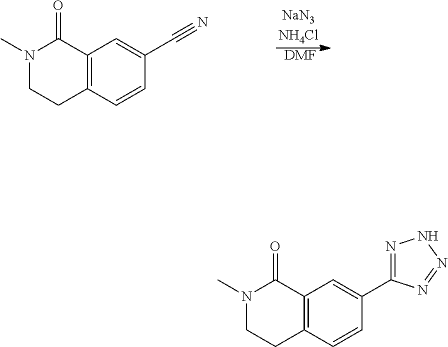

A mixture of 2-methyl-1-oxo-3,4-dihydroisoquinoline-7-carbonitrile (234 mg, 1.26 mmol, 1 equiv.), sodium azide (163 mg, 2.51 mmol, 2 equiv.) and ammonium chloride (134 mg, 2.51 mmol, 2 equiv.) in DMF (3 mL) was stirred at 100° C. Water (15 mL) was added to the reaction mixture followed by HCl 1N. The white solid which precipitated was filtered, washed with water and dried. The product was used in the next step without further purification.

Step C

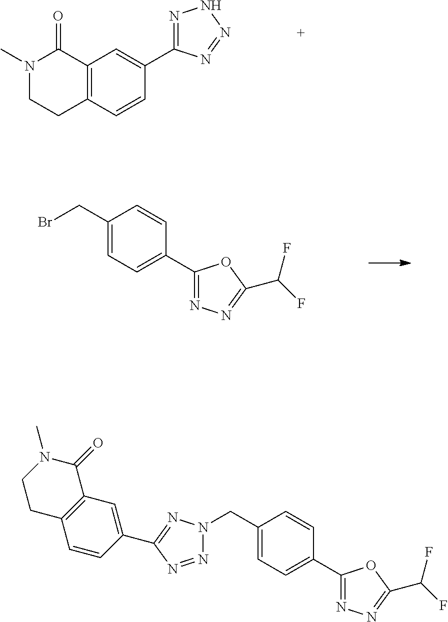 [SMILES: CN1CCc2ccc(-c3nn[nH]n3)cc2C1=O ; FC(F)c1nnc(-c2ccc(CBr)cc2)o1 ; CN1CCc2ccc(-c3nnn(Cc4ccc(-c5nnc(C(F)F)o5)cc4)n3)cc2C1=O]

2-methyl-7-(2H-tetrazol-5-yl)-3,4-dihydroisoquinolin-1-one (100 mg, 0.436 mmol, 1 equiv.) and potassium carbonate (66 mg, 0.48 mmol, 1.1 equiv.) were suspended in DMF (1.5 mL). After 15 min 2-[4-(bromomethyl)phenyl]-5-(difluoromethyl)-1,3,4-oxadiazole (Intermediate B, 126 mg, 0.436 mmol, 1 equiv.) was added to the suspension and the reaction mixture was stirred at r.t. for 1 hour. Full conversion was observed by LC-MS. Water was added to the reaction mixture and the product was extracted into ethyl acetate. The organic phase was washed several times with sat. aq. NaHCO₃ and brine. After concentration under reduced pressure the residue was purified by prep-HPLC using neutral conditions. Pure product (m/z 438.07 [MH+]) was isolated as a white solid (95 mg, 0.217 mmol, 50% yield).

### Example 18. Synthesis of 7-(2-(1-(4-(5-(difluoromethyl)-1,3,4-oxadiazol-2-yl)phenyl)ethyl)-2H-tetrazol-5-yl)-2-methyl-3,4-dihydroisoquinolin-1 (2H)-one (Compd. 229)

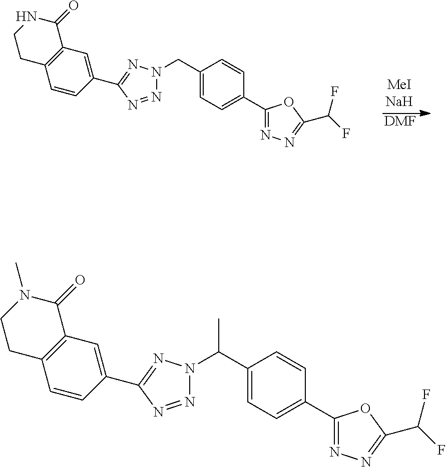

Sodium hydride (5 mg, 0.124 mmol, 1.05 equiv.) was added to a solution of 7-[2-[[4-[5-(difluoromethyl)-1,3,4-oxadiazol-2-yl]phenyl]methyl]tetrazol-5-yl]-3,4-dihydro-2H-isoquinolin-1-one (compd. 83, 50 mg, 0.118 mmol, 1 equiv.) in DMF at r.t. After 30 min methyl iodide (18 mg, 0.130 mmol, 1.1 equiv.) was added and reaction mixture was stirred for 4 h at r.t. Additional 0.5 equiv. of sodium hydride and 1 equiv. of methyl iodide were added. The reaction mixture was stirred overnight at r.t., and then diluted with EtOAc, washed with NaHCO₃ (4 times) and brine. The organic phase was dried over Na₂SO₄, filtered and evaporated in vacuum. Crude was purified by prep-HPLC and pure product (m/z 452.03 [MH+]) was obtained (4 mg, 0.008 mmol, 6.5% yield).

### Example 19. Synthesis of N-(3-(2-(4-(5-(difluoromethyl)-1,3,4-oxadiazol-2-yl)benzyl)-2H-tetrazol-5-yl)phenyl)benzamide (Compd. 156)

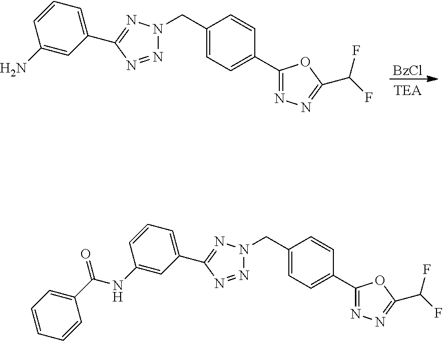

Benzoyl chloride (42 mg, 0.298, 1.1 equiv.) and triethylamine (0.046 mL, 0.325 mmol, 1.2 equiv.) were added to a solution of 3-[2-[[4-[5-(difluoromethyl)-1,3,4-oxadiazol-2-yl]phenyl]methyl]tetrazol-5-yl]aniline (compd. 129, 100 mg, 0.271 mmol, 1 equiv.) in DMF (2 mL). The reaction mixture was stirred at r.t. overnight, then diluted with water. Precipitation occurred. Solid was recovered by filtration and purified by prep-HPLC. Pure product (m/z 474.12 [MH+]) was obtained (31 mg, 0.064 mmol, 24% yield).

### Example 20. Synthesis of 1-(4-(2-(4-(5-(difluoromethyl)-1,3,4-oxadiazol-2-yl)benzyl)-2H-tetrazol-5-yl)piperidin-1-yl)ethan-1-one (Compd. 257)

Step A

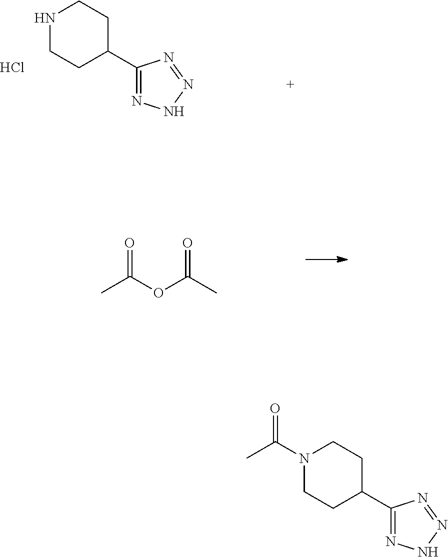

To a solution of 4-(2H-tetrazol-5-yl)piperidine hydrochloride (125 mg, 0.659 mmol, 1 equiv.) in pyridine (1 mL) acetic anhydride (0.075 mL, 0.791 mmol, 1.2 equiv.) was added. The reaction mixture was stirred at 60° C. overnight. Solvent was evaporated under reduced pressure and crude was used for the next step without any purification.

Step B

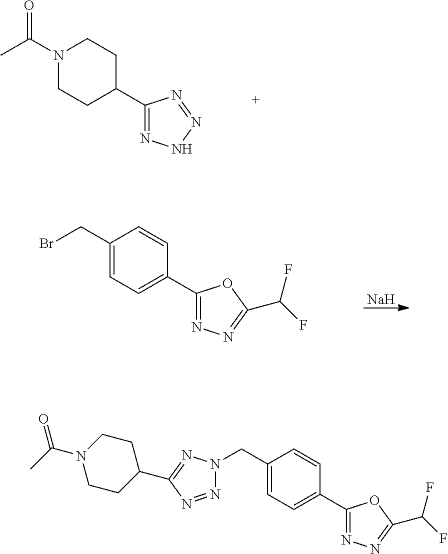

1-[4-(2H-tetrazol-5-yl)piperidin-1-yl]ethanone (128 mg, 0.656 mmol, 1 equiv.) and sodium hydride (65.6 mg, 1.64 mmol, 2.5 equiv.) were suspended in DMF (2 mL) and stirred to obtained clear solution. Then 2-[4-(bromomethyl)phenyl]-5-(difluoromethyl)-1,3,4-oxadiazole (Intermediate B, 208.5 mg, 0.721 mmol, 1.1 equiv.) was added and the reaction mixture was stirred overnight at r.t. Full conversion was observed by LC-MS. Reaction mixture was diluted with water and precipitation occurred. Solid was filtered and purified by prep-HPLC. Pure product (m/z 404.25 [MH+]) was obtained (16.6 mg, 0.041 mmol, 6.2% yield).

### Example 21. Synthesis of 3-(2-(4-(5-(difluoromethyl)-1,3,4-oxadiazol-2-yl)benzyl)-2H-tetrazol-5-yl)-4-fluoro-N,N-dimethylbenzenesulfonamide (Compd. 163)

Step A

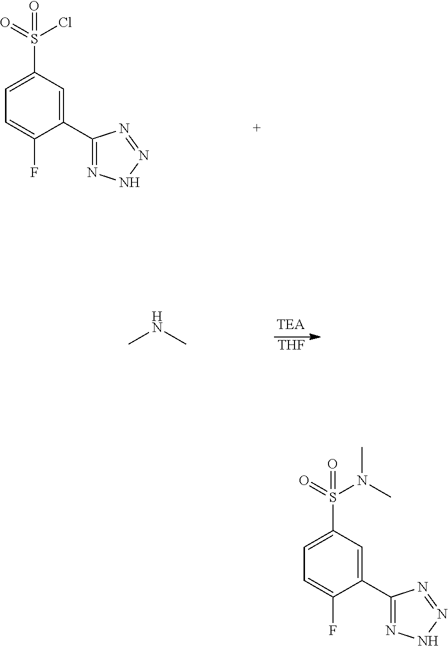

Dimethylamine (0.21 mL, 0.419 mmol, 1.1 equiv.) was added dropwise at −20° C. to a solution of 4-fluoro-3-(2H-tetrazol-5-yl)benzenesulfonyl chloride (100 mg, 0.381 mmol, 1 equiv.) and triethylamine (0.58 mL, 0.419 mmol, 1.1 equiv.) in THF (3 mL). The reaction mixture was stirred for 15 min at −20° C., then warmed to 0° C. Full conversion was observed by LC-MS after 1 h. Solvent was evaporated under reduced pressure. The residue was dissolved in EtOH, then the solvent was evaporated under reduced pressure. Crude was used for the next step without purification.

Step B

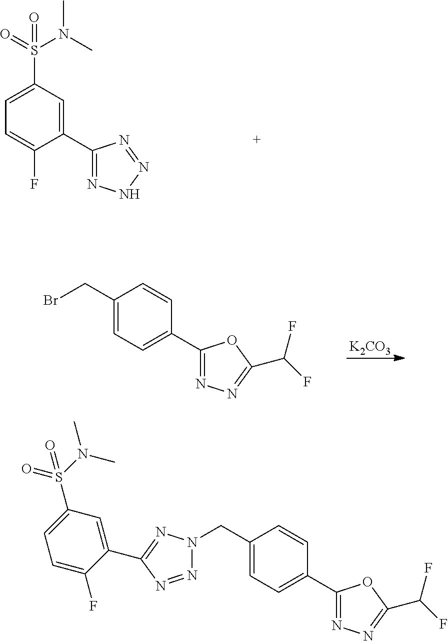

4-fluoro-N,N-dimethyl-3-(2H-tetrazol-5-yl)benzenesulfonamide (60 mg, 0.221 mmol, 1 equiv.) and potassium carbonate (61.14 mg, 0.442 mmol, 2 equiv.) were suspended in DMF (2 mL). After 30 min 2-[4-(bromomethyl)phenyl]-5-(difluoromethyl)-1,3,4-oxadiazole (Intermediate B, 63.94 mg, 0.221 mmol, 1 equiv.) was added to the suspension and the reaction mixture was stirred at r.t. overnight. Full conversion was observed by LC-MS. The reaction mixture was diluted with EtOH, washed with NaHCO₃ and brine, dried over Na₂SO₄, filtered and evaporated under reduced pressure. Crude was purified by prep-HPLC, affording pure product (24 mg, 0.05 mmol, 25% yield, m/z 479.93 [MH+]).

### Example 22. Synthesis of N-(5-(2-((5-(5-(difluoromethyl)-1,3,4-oxadiazol-2-yl)pyrimidin-2-yl)methyl)-2H-tetrazol-5-yl)pyridin-2-yl)-2,2-difluoroacetamide (Compd. 68)

Step A

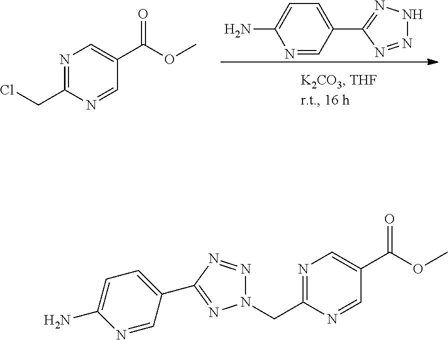 [SMILES: COC(=O)c1cnc(CCl)nc1 ; Nc1ccc(-c2nn[nH]n2)cn1 ; COC(=O)c1cnc(Cn2nnc(-c3ccc(N)nc3)n2)nc1]

Sodium hydride (1.7 equiv.) was added to a solution of 5-(2H-tetrazol-5-yl)pyridin-2-amine (50 mg, 0.31 mmol, 1 equiv.) in 2 mL THF at r.t. The reaction mixture was stirred at r.t. for 2 h. Methyl 2-(chloromethyl)pyrimidine-5-carboxylate (1 equiv.) was added to the reaction mixture, which was stirred at r.t. overnight. Conversion was monitored by LC-MS, detecting the formation of both 2,5- and 1,5-substituted regioisomers. The reaction mixture was diluted with EtOAc, washed with water, sat. aq. NaHCO₃ (4 times) and brine, dried over MgSO₄, evaporated and dried under vacuum to obtain almost pure compound (77 mg, 0.25 mmol, 80% yield), that was used in the next step without additional purification. A 9:1 regioisomeric ratio was determined by NMR.

Step B

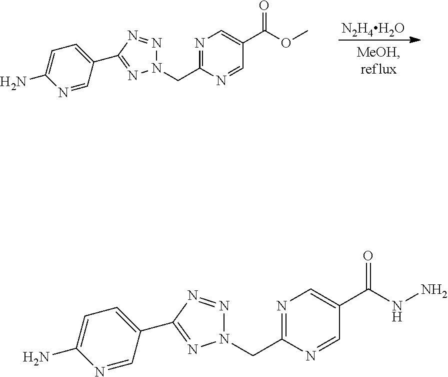

A suspension of methyl 2-[[5-(6-aminopyridin-3-yl)tetrazol-2-yl]methyl]pyrimidine-5-carboxylate (75 mg, 0.24 mmol, 1 equiv.) and hydrazine monohydrate (5 equiv.) in MeOH (2 mL) was stirred at 70° C. over 6 h. Full conversion to the desired product was observed by TLC (DCM/MeOH 95:5) and confirmed by LC-MS. The reaction mixture was evaporated under vacuum and reevaporated from acetonitrile to give target compound (75 mg, 0.24 mmol, 100% yield). The product was used in the subsequent step without further purification.

Step C

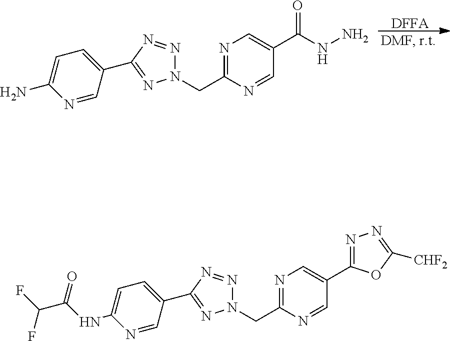

Difluoroacetic anhydride (1 equiv.) was added in portions to a solution of 2-((5-(6-aminopyridin-3-yl)-2H-tetrazol-2-yl)methyl)pyrimidine-5-carbohydrazide (75 mg, 0.24 mmol, 1 equiv.) in DMF (2 mL). After 30 min all the starting material was converted to open intermediate difluoroacetyl hydrazide. Cyclization of the oxadiazole ring and concomitant aminopyridine acylation was performed by addition of extra difluoroacetic anhydride in portions (4×1 equiv.), monitoring conversion by LC-MS. The reaction mixture was diluted with EtOAc, washed with sat. aq. NaHCO₃ (4 times) and brine, dried over Na₂SO₄, evaporated and dried under vacuum. Crude product was purified by prep-HPLC, thus obtaining pure target compound (25 mg, 0.056 mmol, 23% yield, m/z 451.10 [MH+]).

### Example 23. Synthesis of 5-(1-((5-(5-(difluoromethyl)-1,3,4-oxadiazol-2-yl)pyridin-2-yl)methyl)-1H-1,2,3-triazol-4-yl)pyridin-2-amine (Compd. 5)

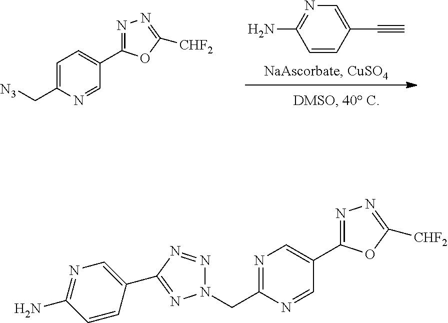

Copper(II) sulfate pentahydrate (19 mg, 0.3 equiv., 0.5 M aqueous solution) and sodium L-ascorbate (25 mg, 0.5 equiv., 1 M aqueous solution) were added to a solution of 2-(6-(azidomethyl)pyridin-3-yl)-5-(difluoromethyl)-1,3,4-oxadiazole (Intermediate F, 70 mg, 0.279 mmol, 1.1 equiv.) and 5-ethynylpyridin-2-amine (30 mg, 0.251 mmol, 1 equiv.) in 1 mL DMSO. The reaction mixture was agitated at 40° C. over 2 h. Full conversion of the starting material was detected by LC-MS. Reaction mixture was filtered through syringe filter and submitted to prep-HPLC without any further work up. After evaporation of fractions 45 mg of target compound (0.122 mmol, 48% yield) were obtained as off-white solid (m/z 371.11 [MH+]).

The following compounds were synthesized according to the same procedure:

| Compd. | Structure | m/z [MH+] |
|---|---|---|
| 6 | 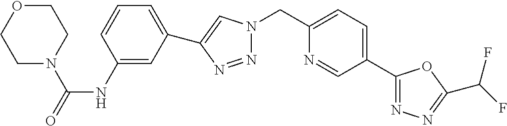 [SMILES: O=C(Nc1cccc(-c2cn(Cc3ccc(-c4nnc(C(F)F)o4)cn3)nn2)c1)N1CCOCC1] | 483.11 |
| 44 | 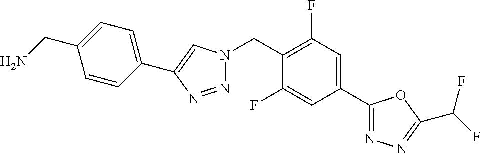 [SMILES: NCc1ccc(-c2cn(Cc3c(F)cc(-c4nnc(C(F)F)o4)cc3F)nn2)cc1] | 419.7 |
| 52 | 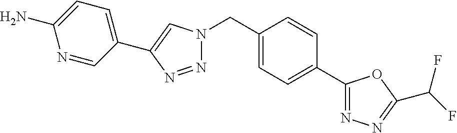 [SMILES: Nc1ccc(-c2cn(Cc3ccc(-c4nnc(C(F)F)o4)cc3)nn2)cn1] | 370.7 |
| 63 | 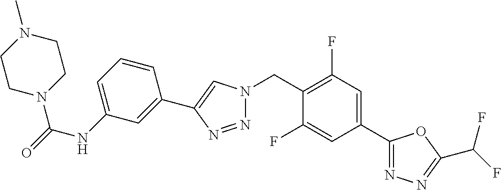 [SMILES: CN1CCN(C(=O)Nc2cccc(-c3cn(Cc4c(F)cc(-c5nnc(C(F)F)o5)cc4F)nn3)c2)CC1] | 531.3 |
| 67 | 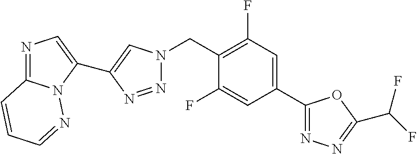 [SMILES: Fc1cc(-c2nnc(C(F)F)o2)cc(F)c1Cn1cc(-c2cnc3cccnn23)nn1] | 431.6 |
| 70 | 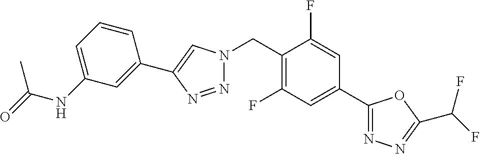 [SMILES: CC(=O)Nc1cccc(-c2cn(Cc3c(F)cc(-c4nnc(C(F)F)o4)cc3F)nn2)c1] | 447.12 |
| 92 | 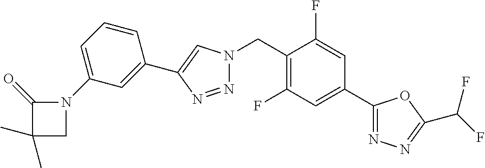 [SMILES: CC1(C)CN(c2cccc(-c3cn(Cc4c(F)cc(-c5nnc(C(F)F)o5)cc4F)nn3)c2)C1=O] | 487.4 |
| 105 | 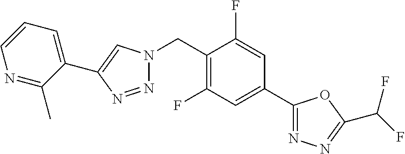 [SMILES: Cc1ncccc1-c1cn(Cc2c(F)cc(-c3nnc(C(F)F)o3)cc2F)nn1] | 405.12 |
| 121 | 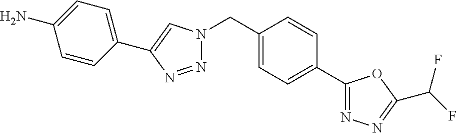 [SMILES: Nc1ccc(-c2cn(Cc3ccc(-c4nnc(C(F)F)o4)cc3)nn2)cc1] | 369.06 |
| 122 | 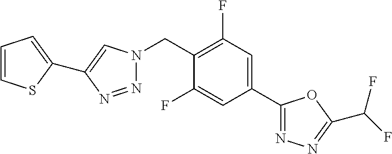 [SMILES: Fc1cc(-c2nnc(C(F)F)o2)cc(F)c1Cn1cc(-c2cccs2)nn1] | 396.06 |
| 131 | 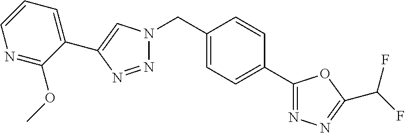 [SMILES: COc1ncccc1-c1cn(Cc2ccc(-c3nnc(C(F)F)o3)cc2)nn1] | 385.14 |
| 138 | 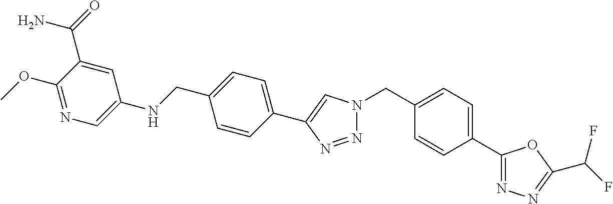 [SMILES: COc1ncc(NCc2ccc(-c3cn(Cc4ccc(-c5nnc(C(F)F)o5)cc4)nn3)cc2)cc1C(N)=O] | 533.6 |
| 139 | 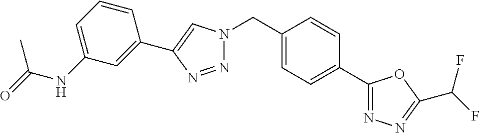 [SMILES: CC(=O)Nc1cccc(-c2cn(Cc3ccc(-c4nnc(C(F)F)o4)cc3)nn2)c1] | 411.12 |
| 146 | 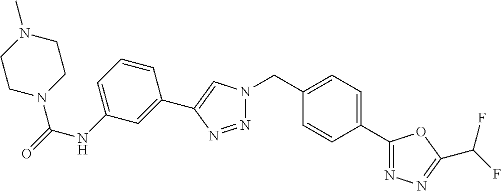 [SMILES: CN1CCN(C(=O)Nc2cccc(-c3cn(Cc4ccc(-c5nnc(C(F)F)o5)cc4)nn3)c2)CC1] | 495.36 |
| 169 | 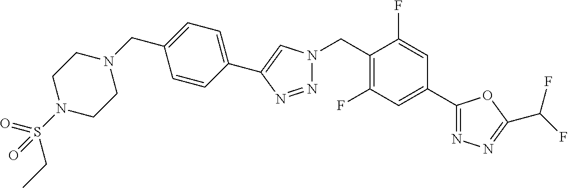 [SMILES: CCS(=O)(=O)N1CCN(Cc2ccc(-c3cn(Cc4c(F)cc(-c5nnc(C(F)F)o5)cc4F)nn3)cc2)CC1] | 580.6 |
| 175 | 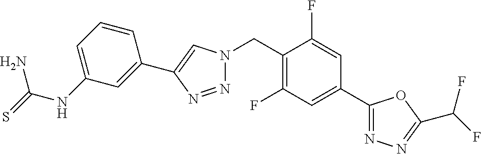 [SMILES: NC(=S)Nc1cccc(-c2cn(Cc3c(F)cc(-c4nnc(C(F)F)o4)cc3F)nn2)c1] | 464.3 |
| 179 | 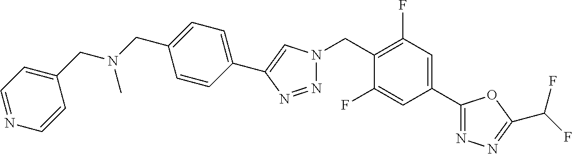 [SMILES: CN(Cc1ccncc1)Cc1ccc(-c2cn(Cc3c(F)cc(-c4nnc(C(F)F)o4)cc3F)nn2)cc1] | 524.22 |
| 180 | 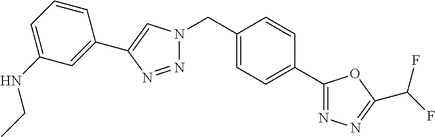 [SMILES: CCNc1cccc(-c2cn(Cc3ccc(-c4nnc(C(F)F)o4)cc3)nn2)c1] | 397.4 |
| 183 | 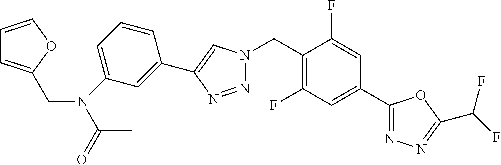 [SMILES: CC(=O)N(Cc1ccco1)c1cccc(-c2cn(Cc3c(F)cc(-c4nnc(C(F)F)o4)cc3F)nn2)c1] | 527.6 |
| 192 | 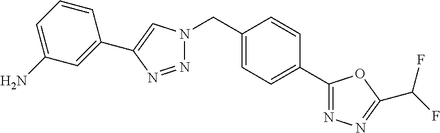 [SMILES: Nc1cccc(-c2cn(Cc3ccc(-c4nnc(C(F)F)o4)cc3)nn2)c1] | 369.06 |
| 217 | 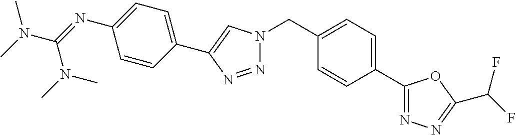 [SMILES: CN(C)C(=Nc1ccc(-c2cn(Cc3ccc(-c4nnc(C(F)F)o4)cc3)nn2)cc1)N(C)C] | 476.6 |
| 233 | 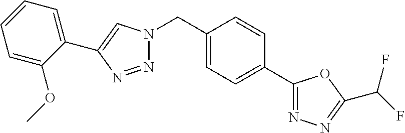 [SMILES: COc1ccccc1-c1cn(Cc2ccc(-c3nnc(C(F)F)o3)cc2)nn1] | 384.06 |
| 239 | 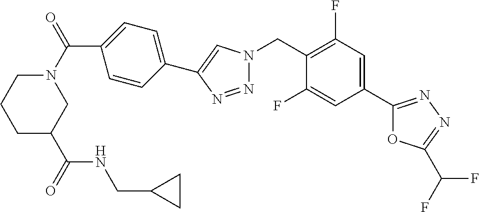 [SMILES: O=C(NCC1CC1)C1CCCN(C(=O)c2ccc(-c3cn(Cc4c(F)cc(-c5nnc(C(F)F)o5)cc4F)nn3)cc2)C1] | 598.6 |
| 241 | 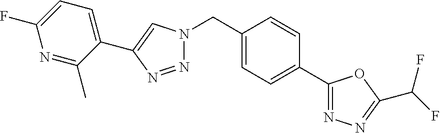 [SMILES: Cc1nc(F)ccc1-c1cn(Cc2ccc(-c3nnc(C(F)F)o3)cc2)nn1] | 387.3 |
| 248 | 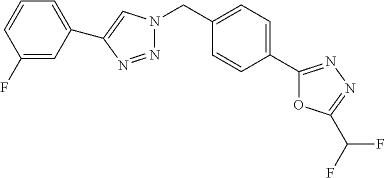 [SMILES: Fc1cccc(-c2cn(Cc3ccc(-c4nnc(C(F)F)o4)cc3)nn2)c1] | 372 |
| 275 | 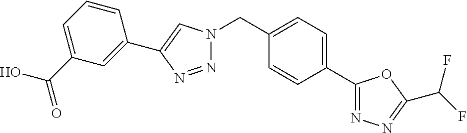 [SMILES: O=C(O)c1cccc(-c2cn(Cc3ccc(-c4nnc(C(F)F)o4)cc3)nn2)c1] | 439.1* |
| 280 | 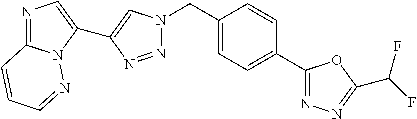 [SMILES: FC(F)c1nnc(-c2ccc(Cn3cc(-c4cnc5cccnn45)nn3)cc2)o1] | 395.6 |
| 281 | 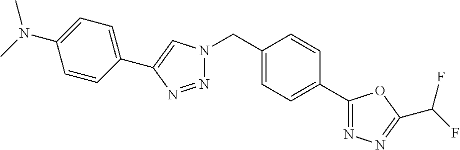 [SMILES: CN(C)c1ccc(-c2cn(Cc3ccc(-c4nnc(C(F)F)o4)cc3)nn2)cc1] | 397.14 |
| 282 | 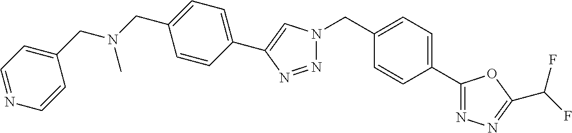 [SMILES: CN(Cc1ccncc1)Cc1ccc(-c2cn(Cc3ccc(-c4nnc(C(F)F)o4)cc3)nn2)cc1] | 488.28 |
| 283 | 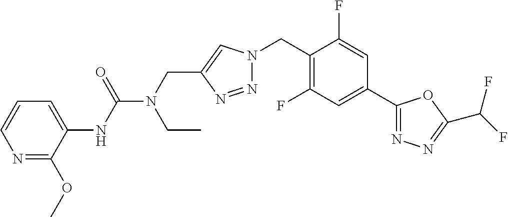 [SMILES: CCN(Cc1cn(Cc2c(F)cc(-c3nnc(C(F)F)o3)cc2F)nn1)C(=O)Nc1cccnc1OC] | 521.9 |
| 296 | 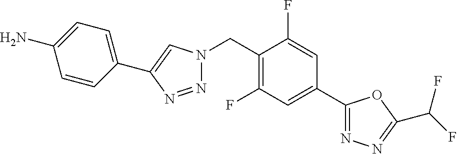 [SMILES: Nc1ccc(-c2cn(Cc3c(F)cc(-c4nnc(C(F)F)o4)cc3F)nn2)cc1] | 405.06 |
| 297 | 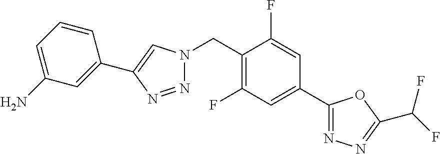 [SMILES: Nc1cccc(-c2cn(Cc3c(F)cc(-c4nnc(C(F)F)o4)cc3F)nn2)c1] | 405.06 |

*[M + ACN + H]⁺ was observed.

### Example 24. Synthesis of 5-(1-(4-(5-(difluoromethyl)-1,3,4-oxadiazol-2-yl)-2,3-difluorobenzyl)-1H-1,2,3-triazol-4-yl)pyridin-2-amine (Compd. 21)

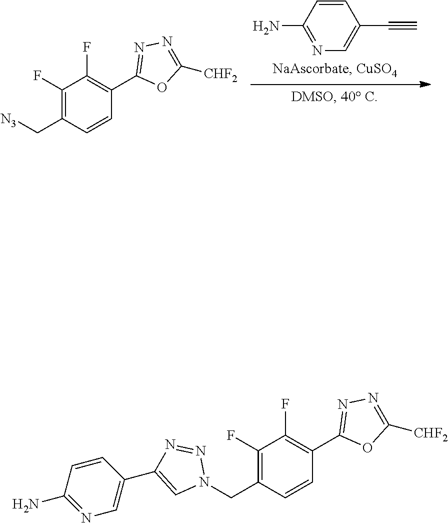

Copper(II) sulfate pentahydrate (4 mg, 0.1 equiv., 0.5 M aqueous solution) and sodium L-ascorbate (16 mg, 0.5 equiv., 1 M aqueous solution) were added to a solution of 2-(4-(azidomethyl)-2,3-difluorophenyl)-5-(difluoromethyl)-1,3,4-oxadiazole (Intermediate H, 48 mg, 0.168 mmol, 1 equiv.) and 5-ethynylpyridin-2-amine (20 mg, 0.168 mmol, 1 equiv.) in 1 mL DMSO. The reaction mixture was agitated at 40° C. over 2 h. Full conversion of the starting material was detected by LC-MS. Reaction mixture was filtered through syringe filter and submitted to prep-HPLC without any further work up. After evaporation of fractions 19 mg of target compound (0.05 mmol, 28% yield) were obtained as off-white solid (m/z 406.10 [MH+]).

The following compounds were synthesized according to the same synthetic route:

| Compd. | Structure | m/z [MH+] |
|---|---|---|
| 15 | 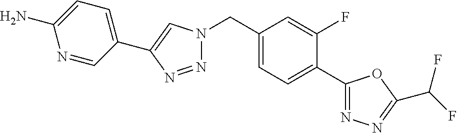 [SMILES: Nc1ccc(-c2cn(Cc3ccc(-c4nnc(C(F)F)o4)c(F)c3)nn2)cn1] | 388.7 |
| 16 | 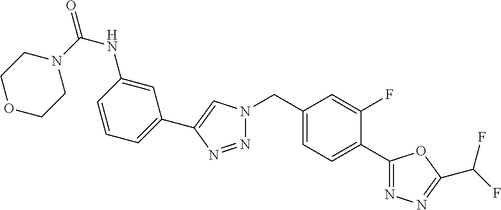 [SMILES: O=C(Nc1cccc(-c2cn(Cc3ccc(-c4nnc(C(F)F)o4)c(F)c3)nn2)c1)N1CCOCC1] | 500.5 |
| 39 | 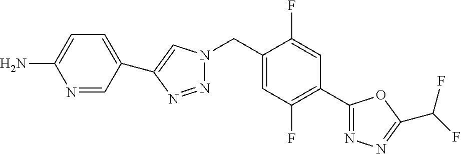 [SMILES: Nc1ccc(-c2cn(Cc3cc(F)c(-c4nnc(C(F)F)o4)cc3F)nn2)cn1] | 406.5 |
| 42 | 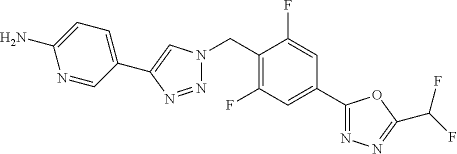 [SMILES: Nc1ccc(-c2cn(Cc3c(F)cc(-c4nnc(C(F)F)o4)cc3F)nn2)cn1] | 406.09 |
| 47 | 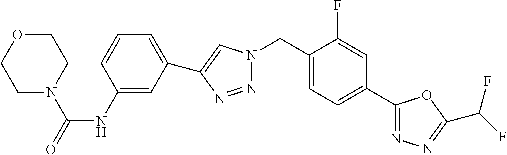 [SMILES: O=C(Nc1cccc(-c2cn(Cc3ccc(-c4nnc(C(F)F)o4)cc3F)nn2)c1)N1CCOCC1] | 500.3 |
| 48 | 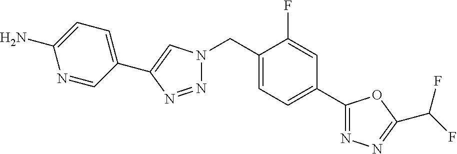 [SMILES: Nc1ccc(-c2cn(Cc3ccc(-c4nnc(C(F)F)o4)cc3F)nn2)cn1] | 388.12 |
| 73 | 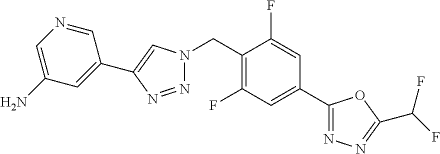 [SMILES: Nc1cncc(-c2cn(Cc3c(F)cc(-c4nnc(C(F)F)o4)cc3F)nn2)c1] | 406.12 |
| 78 | 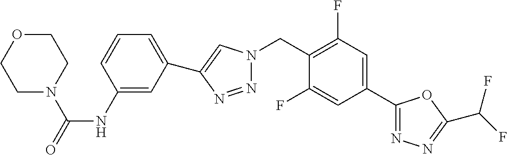 [SMILES: O=C(Nc1cccc(-c2cn(Cc3c(F)cc(-c4nnc(C(F)F)o4)cc3F)nn2)c1)N1CCOCC1] | 518.11 |
| 114 | 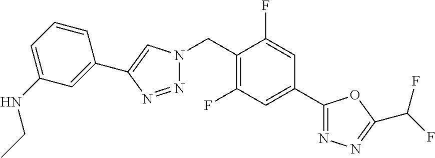 [SMILES: CCNc1cccc(-c2cn(Cc3c(F)cc(-c4nnc(C(F)F)o4)cc3F)nn2)c1] | 433.4 |
| 134 | 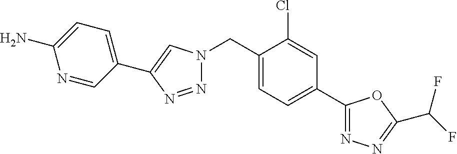 [SMILES: Nc1ccc(-c2cn(Cc3ccc(-c4nnc(C(F)F)o4)cc3Cl)nn2)cn1] | 404.4 |
| 214 | 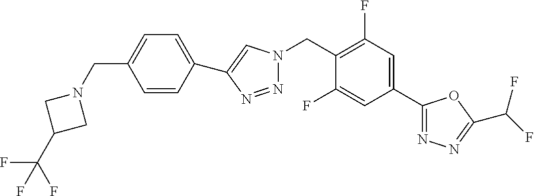 [SMILES: Fc1cc(-c2nnc(C(F)F)o2)cc(F)c1Cn1cc(-c2ccc(CN3CC(C(F)(F)F)C3)cc2)nn1] | 527.4 |

### Example 25. Synthesis of 5-(1-((6-(5-(difluoromethyl)-1,3,4-oxadiazol-2-yl)pyridazin-3-yl)methyl)-1H-1,2,3-triazol-4-yl)pyridin-2-amine (Compd. 198)

Step A

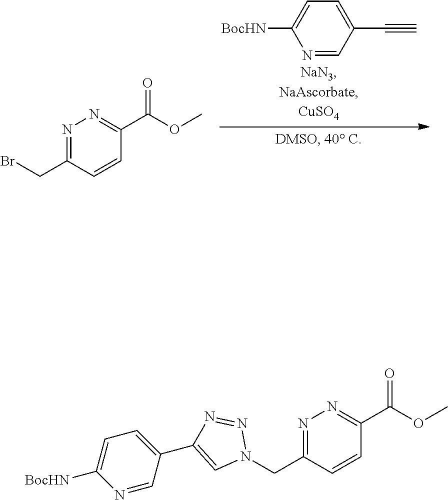 [SMILES: C#Cc1ccc(NC(=O)OC(C)(C)C)nc1 ; COC(=O)c1ccc(CBr)nn1 ; COC(=O)c1ccc(Cn2cc(-c3ccc(NC(=O)OC(C)(C)C)nc3)nn2)nn1]

Copper(II) sulfate pentahydrate 0.5 M aq. solution (234 μL, 0.3 equiv.) and sodium L-ascorbate 1.0 M aq. solution (195 μL, 0.5 equiv.) were added to a stirring solution of methyl 6-(bromomethyl)pyridazine-3-carboxylate (1 equiv.), tert-butyl (5-ethynylpyridin-2-yl)carbamate (85 mg, 0.389 mmol, 1 equiv.) and sodium azide (1.1 equiv) in DMSO (2 mL). The resulting mixture was stirred at r.t. for 1 h. Additional 0.4 equiv. of methyl 6-(bromomethyl)pyridazine-3-carboxylate were added within 2 h to reach full conversion, which was monitored by LC-MS. The reaction mixture was diluted with EtOAc, washed with water, sat. aq. NaHCO₃ (3 times) and brine, dried and evaporated under reduced pressure. The residue obtained was used in the next step without purification (115 mg, 0.28 mmol, 72% yield).

Step B

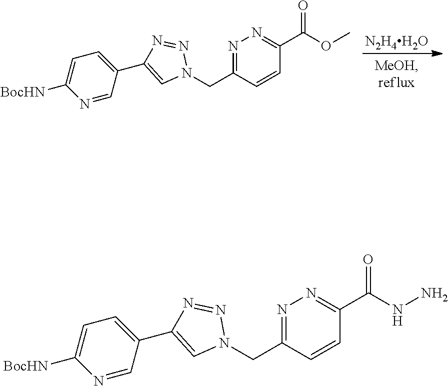

A suspension of methyl 6-((4-(6-((tert-butoxycarbonyl)amino)pyridin-3-yl)-1H-1,2,3-triazol-1-yl)methyl)pyridazine-3-carboxylate (115 mg, 0.28 mmol, 1 equiv.) and hydrazine monohydrate (5 equiv.) in MeOH (5 mL) was stirred at 70° C. over 3 h. Full conversion to the desired product was observed by LC-MS. The reaction mixture was evaporated in vacuum and reevaporated with acetonitrile to give target compound as off-white suspension (115 mg, 0.28 mmol, 100% yield). The product was used for the subsequent step without further purification.

Step C

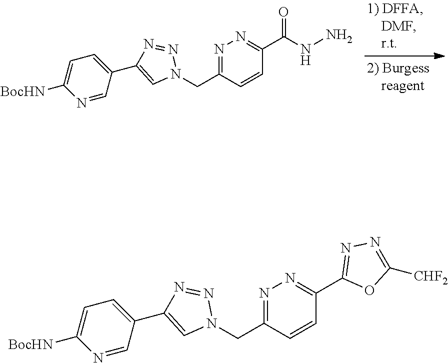 [SMILES: CC(C)(C)OC(=O)Nc1ccc(-c2cn(Cc3ccc(C(=O)NN)nn3)nn2)cn1 ; CC(C)(C)OC(=O)Nc1ccc(-c2cn(Cc3ccc(-c4nnc(C(F)F)o4)nn3)nn2)cn1]

Difluoroacetic anhydride (3 equiv.) was added to a solution of tert-butyl(5-(1-((6-(hydrazinecarbonyl)pyridazin-3-yl)methyl)-1H-1,2,3-triazol-4-yl)pyridin-2-yl)carbamate (35 mg, 0.085 mmol, 1 equiv.) in DMF (2 mL). After 30 min all the starting material was converted to open intermediate. Some Boc deprotected/difluoroacylated side reaction occurs. Cyclization was performed by addition of Burgess reagent (3 equiv.+1 equiv. until completion), monitoring conversion by LC-MS. The reaction mixture was diluted with EtOAc, washed with sat. aq. NaHCO₃ (4 times) and brine, dried over Na₂SO₄, evaporated and dried under vacuum. Almost pure target compound obtained (22 mg, 0.047 mmol, 54% yield) could be used in the next step without purification.

Step D

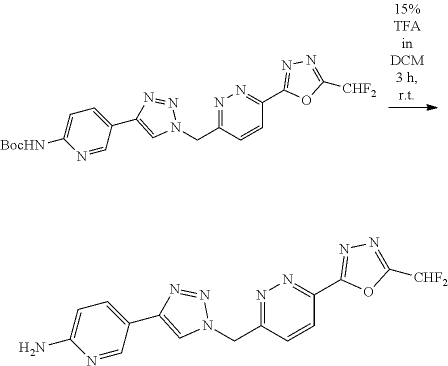 [SMILES: CC(C)(C)OC(=O)Nc1ccc(-c2cn(Cc3ccc(-c4nnc(C(F)F)o4)nn3)nn2)cn1 ; Nc1ccc(-c2cn(Cc3ccc(-c4nnc(C(F)F)o4)nn3)nn2)cn1]

A solution of tert-butyl(5-(1-((6-(5-(difluoromethyl)-1,3,4-oxadiazol-2-yl)pyridazin-3-yl)methyl)-1H-1,2,3-triazol-4-yl)pyridin-2-yl)carbamate (15 mg, 0.032 mmol, 1 equiv.) and TFA (50 μL) in DCM (300 μL) was stirred at r.t. over 3 h. Full conversion was detected by LC-MS. Reaction mixture was diluted with EtOAc, washed with sat. aq. NaHCO₃ (twice) and brine, dried over Na₂SO₄ and evaporated under vacuum. The residue obtained was purified by prep-HPLC. Target compound (3 mg, 0.007 mmol, 22% yield) was obtained as a white solid (m/z 372.11 [MH+]).

### Example 26. Synthesis of 6-(1-(4-(5-(difluoromethyl)-1,3,4-oxadiazol-2-yl)benzyl)-1H-1,2,3-triazol-4-yl)isoindolin-1-one (Compd. 109)

Step A

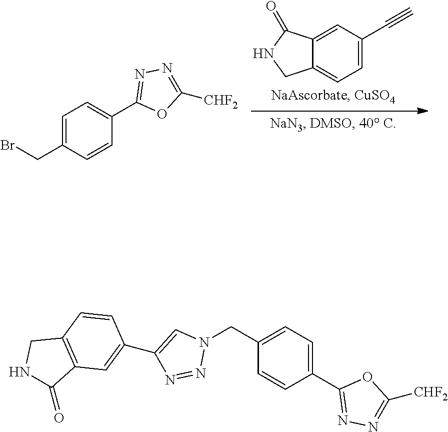 [SMILES: FC(F)c1nnc(-c2ccc(CBr)cc2)o1 ; C#Cc1ccc2c(c1)C(=O)NC2 ; O=C1NCc2ccc(-c3cn(Cc4ccc(-c5nnc(C(F)F)o5)cc4)nn3)cc21]

Copper(II) sulfate pentahydrate 0.5 M aq. solution (572 μL, 0.3 equiv.) and sodium L-ascorbate 1.0 M aq. solution (477 μL, 0.5 equiv.) were added to a stirring solution of 2-[4-(bromomethyl)phenyl]-5-(difluoromethyl)-1,3,4-oxadiazole (Intermediate B, 1 equiv.), 6-ethynyl-2,3-dihydroisoindol-1-one (150 mg, 0.954 mmol, 1 equiv.) and sodium azide (1 equiv.) in DMSO (2 mL). The resulting mixture was stirred at room temperature for 30 minutes. The reaction mixture was diluted with water and precipitate was filtered off. Purification by prep-HPLC (C18, water/ACN) gave product in 32% yield (132 mg, 0.32 mmol, m/z 409.11 [MH+]).

The following compounds were synthesized according to the same procedure:

| Compd. | Structure | m/z [MH+] |
|---|---|---|
| 34 | 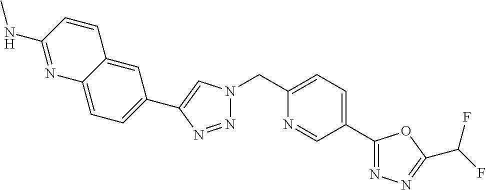 [SMILES: CNc1ccc2cc(-c3cn(Cc4ccc(-c5nnc(C(F)F)o5)cn4)nn3)ccc2n1] | 435.09 |
| 37 | 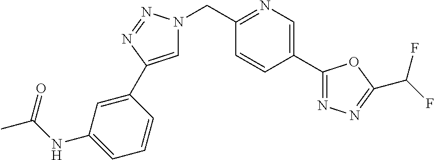 [SMILES: CC(=O)Nc1cccc(-c2cn(Cc3ccc(-c4nnc(C(F)F)o4)cn3)nn2)c1] | 412.04 |
| 53 | 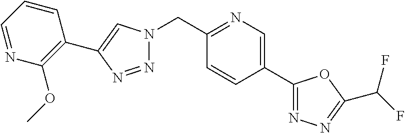 [SMILES: COc1ncccc1-c1cn(Cc2ccc(-c3nnc(C(F)F)o3)cn2)nn1] | 386.05 |
| 74 | 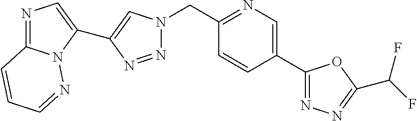 [SMILES: FC(F)c1nnc(-c2ccc(Cn3cc(-c4cnc5cccnn45)nn3)nc2)o1] | 396.01 |
| 85 | 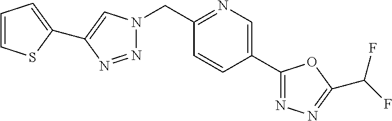 [SMILES: FC(F)c1nnc(-c2ccc(Cn3cc(-c4cccs4)nn3)nc2)o1] | 360.97 |
| 115 | 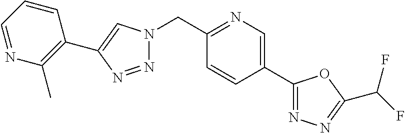 [SMILES: Cc1ncccc1-c1cn(Cc2ccc(-c3nnc(C(F)F)o3)cn2)nn1] | 370.5 |
| 142 | 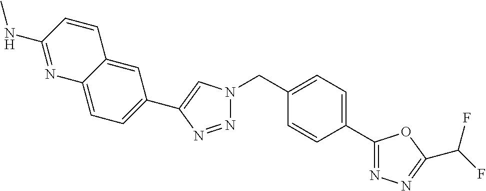 [SMILES: CNc1ccc2cc(-c3cn(Cc4ccc(-c5nnc(C(F)F)o5)cc4)nn3)ccc2n1] | 434.5 |
| 304 | 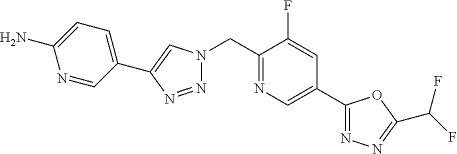 [SMILES: Nc1ccc(-c2cn(Cc3ncc(-c4nnc(C(F)F)o4)cc3F)nn2)cn1] | 389.01 |
| 356 | 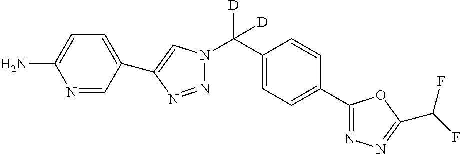 [SMILES: [2H]C([2H])(c1ccc(-c2nnc(C(F)F)o2)cc1)n1cc(-c2ccc(N)nc2)nn1] | 372.19 |
| 362 | 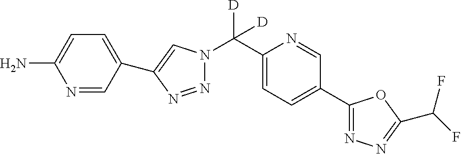 [SMILES: [2H]C([2H])(c1ccc(-c2nnc(C(F)F)o2)cn1)n1cc(-c2ccc(N)nc2)nn1] | 373.14 |
| 381 | 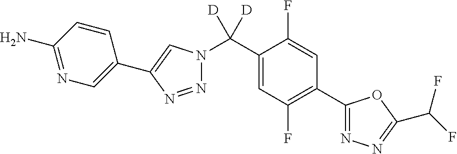 [SMILES: [2H]C([2H])(c1cc(F)c(-c2nnc(C(F)F)o2)cc1F)n1cc(-c2ccc(N)nc2)nn1] | 407.86 |
| 385 | 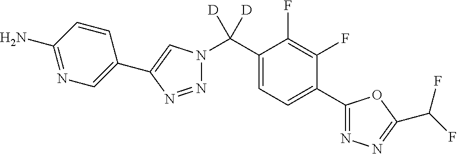 [SMILES: [2H]C([2H])(c1ccc(-c2nnc(C(F)F)o2)c(F)c1F)n1cc(-c2ccc(N)nc2)nn1] | 407.95 |
| 389 | 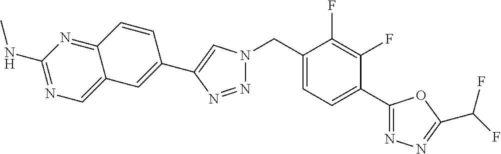 [SMILES: CNc1ncc2cc(-c3cn(Cc4ccc(-c5nnc(C(F)F)o5)c(F)c4F)nn3)ccc2n1] | 471.15 |
| 390 | 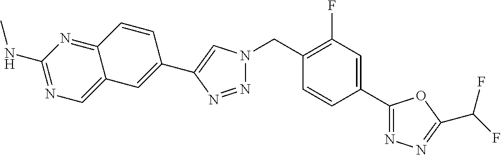 [SMILES: CNc1ncc2cc(-c3cn(Cc4ccc(-c5nnc(C(F)F)o5)cc4F)nn3)ccc2n1] | 453.17 |
| 391 | 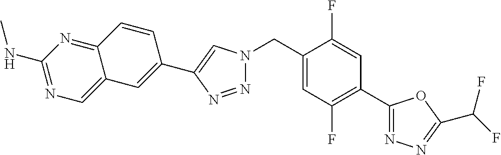 [SMILES: CNc1ncc2cc(-c3cn(Cc4cc(F)c(-c5nnc(C(F)F)o5)cc4F)nn3)ccc2n1] | 471.16 |
| 392 | 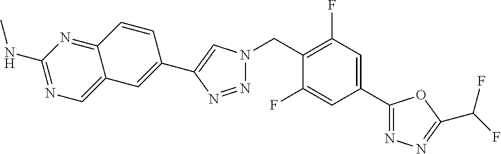 [SMILES: CNc1ncc2cc(-c3cn(Cc4c(F)cc(-c5nnc(C(F)F)o5)cc4F)nn3)ccc2n1] | 471.18 |
| 393 | 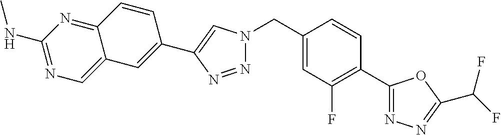 [SMILES: CNc1ncc2cc(-c3cn(Cc4ccc(-c5nnc(C(F)F)o5)c(F)c4)nn3)ccc2n1] | 453.15 |

### Example 27. Synthesis of N-(4-(1-(4-(5-(difluoromethyl)-1,3,4-oxadiazol-2-yl)benzyl)-1H-1,2,3-triazol-4-yl)phenyl)-4,5-dihydro-1H-imidazol-2-amine (Compd. 10)

Step A

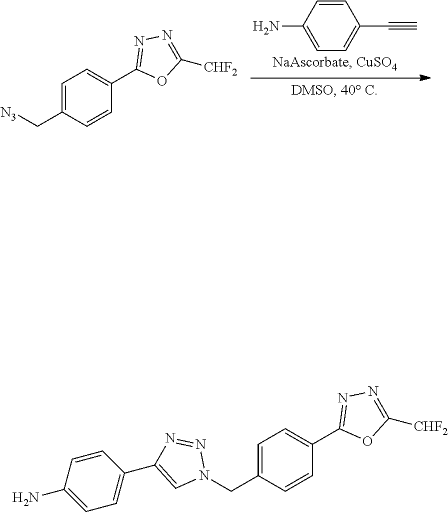

Copper(II) sulfate pentahydrate (0.2 equiv., 0.5 M aqueous solution) and sodium L-ascorbate (0.4 equiv., 1 M aqueous solution) were added to a solution of 2-(4-(azidomethyl)-phenyl)-5-(difluoromethyl)-1,3,4-oxadiazole (Intermediate G,) (60 mg, 0.239 mmol, 1 equiv.) and 5-ethynylpyridin-2-amine (36 mg, 0.311 mmol, 1.3 equiv.) in 1 mL DMSO. The reaction mixture was agitated at 40° C. over 2 h. Full conversion of the starting material was detected by LC-MS. Water was added to the reaction mixture, which was then extracted with ethyl acetate. The organic layer was washed with brine, dried over MgSO₄, filtered and concentrated under reduced pressure to afford a brown solid, which was used directly in the next step (84 mg, 0.228 mmol, 95% yield).

Step B

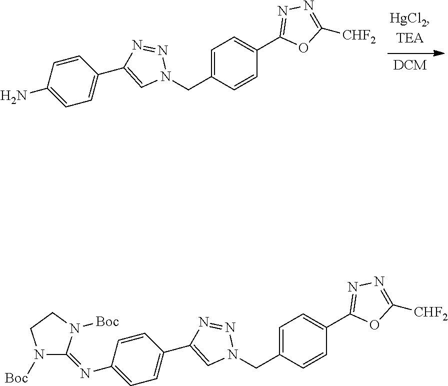 [SMILES: Nc1ccc(-c2cn(Cc3ccc(-c4nnc(C(F)F)o4)cc3)nn2)cc1 ; CC(C)(C)OC(=O)N1CCN(C(=O)OC(C)(C)C)C1=Nc1ccc(-c2cn(Cc3ccc(-c4nnc(C(F)F)o4)cc3)nn2)cc1]

Mercury(II) chloride (1.1 equiv.) was added to a solution of 4-(1-(4-(5-(difluoromethyl)-1,3,4-oxadiazol-2-yl)benzyl)-1H-1,2,3-triazol-4-yl)aniline (84 mg, 0.228 mmol, 1 equiv.), N,N′-di(tertbutoxycarbonyl)imidazolidine-2-thione (1 equiv.) and triethylamine (1.3 equiv.) in 1 mL DCM at 0° C. The resulting mixture was stirred at 0° C. for 1 h and at r.t. for 2 days. The reaction mixture was diluted with water and DCM, filtered and extracted with DCM. The organic layer was washed with brine, dried over MgSO₄, filtered, and concentrated under reduced pressure to afford a yellow oil, which was used directly in the next step (145 mg, 0.228 mmol, 100% yield).

Step C

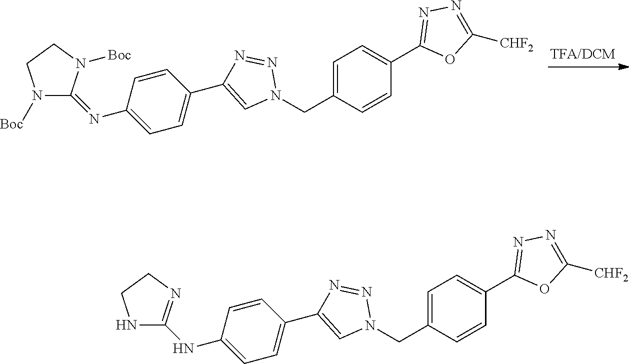 [SMILES: CC(C)(C)OC(=O)N1CCN(C(=O)OC(C)(C)C)C1=Nc1ccc(-c2cn(Cc3ccc(-c4nnc(C(F)F)o4)cc3)nn2)cc1 ; FC(F)c1nnc(-c2ccc(Cn3cc(-c4ccc(NC5=NCCN5)cc4)nn3)cc2)o1]

di-tert-butyl 2-((4-(1-(4-(5-(difluoromethyl)-1,3,4-oxadiazol-2-yl)benzyl)-1H-1,2,3-triazol-4-yl)phenyl)imino)imidazolidine-1,3-dicarboxylate (145 mg, 0.228 mmol, 1 equiv.) was dissolved in 2 mL DCM and TFA (20 equiv.) was added. The reaction mixture was stirred at r.t. overnight. The mixture was diluted with DCM and washed with sat. aq. NaHCO₃ and brine. During washing with brine, precipitation occurred. The solid was filtered, washed with water and dried under vacuum to obtain target compound (55 mg, 0.121 mmol, 53% yield, m/z 436.95 [MH+]).

The following compound was synthesized according to the same procedure:

| Compd. | Structure | m/z [MH+] |
|---|---|---|
| 24 | 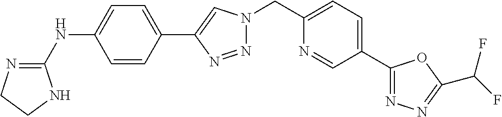 [SMILES: FC(F)c1nnc(-c2ccc(Cn3cc(-c4ccc(NC5=NCCN5)cc4)nn3)nc2)o1] | 437.96 |

### Example 28. Synthesis of (5-(1-(4-(5-(difluoromethyl)-1,3,4-oxadiazol-2-yl)benzyl)-1H-1,2,3-triazol-4-yl)pyridin-2-yl)methanamine (Compd. 155)

Step A

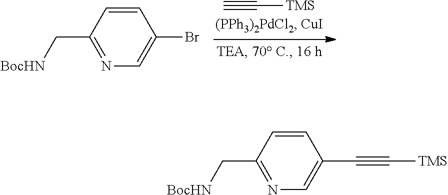

tert-butyl ((5-bromopyridin-2-yl)methyl)carbamate (500 mg, 1.74 mmol, 1 equiv.) was dissolved in triethylamine (9.7 mL, 40 equiv.) and the resulting mixture was degassed. Then ethynyl(trimethyl)silane (1.2 equiv.) was added to the reaction mixture, which was degassed. Bis(triphenylphosphine)palladium (II) chloride (0.02 equiv.) and copper(I) iodide (0.04 equiv.) were added and, after degassing, the reaction mixture was stirred at 70° C. overnight. The mixture was diluted with water and extracted with EtOAc. Combined organic phases were dried over MgSO₄, filtered and concentrated to give a crude product, which was used in the next step without any further purification (530 mg, 1.74 mmol, 100% yield).

Step B

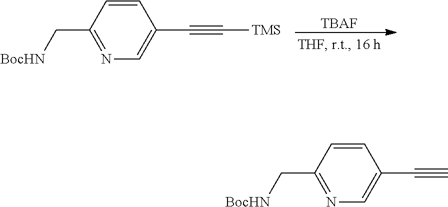 [SMILES: CC(C)(C)OC(=O)NCc1ccc(C#C[Si](C)(C)C)cn1 ; "TBAF" ; C#Cc1ccc(CNC(=O)OC(C)(C)C)nc1]

tert-butyl ((5-((trimethylsilyl)ethynyl)pyridin-2-yl)methyl)carbamate (530 mg, 1.74 mmol, 1 equiv.) was dissolved in 5 mL THF. Tetrabutylammonium fluoride (2 equiv.) was added. The reaction mixture was stirred at r.t. overnight. The reaction mixture was diluted with EtOAc and washed with water. Organic phase was dried over Na₂SO₄ and evaporated. Crude residue was purified by flash column chromatography (0-1% MeOH/DCM) to afford the pure product (305 mg, 1.31 mmol, 74% yield).

Step C

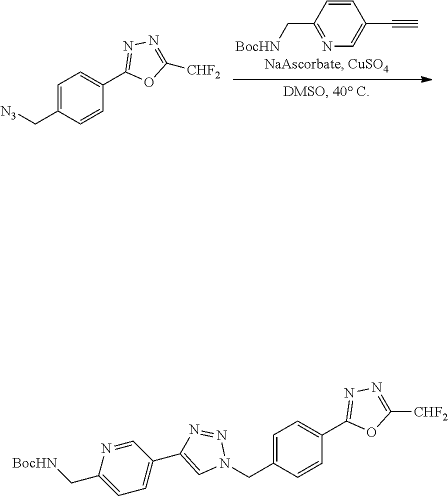 [SMILES: [N-]=[N+]=NCc1ccc(-c2nnc(C(F)F)o2)cc1 ; C#Cc1ccc(CNC(=O)OC(C)(C)C)nc1 ; CC(C)(C)OC(=O)NCc1ccc(-c2cn(Cc3ccc(-c4nnc(C(F)F)o4)cc3)nn2)cn1]

Copper(II) sulfate pentahydrate (0.2 equiv., 0.5 M aqueous solution) and sodium L-ascorbate (0.4 equiv., 1 M aqueous solution) were added to a solution of 2-(4-(azidomethyl)-phenyl)-5-(difluoromethyl)-1,3,4-oxadiazole (Intermediate G, 16 mg, 0.062 mmol, 1.1 equiv.) and tert-butyl N-[(5-ethynylpyridin-2-yl)methyl]carbamate (13 mg, 0.056 mmol, 1 equiv.) in 300 μL DMSO. The reaction mixture was stirred at 40° C. over 2 h. Full conversion of the starting material was detected by LC-MS. Water was added to the reaction mixture and extraction was done with MTBE. The organic layer was washed with brine, dried over MgSO₄, filtered and concentrated under reduced pressure to afford the desired product, which was used in the next step without further purification (23 mg, 0.043 mmol, 76% yield).

Step D

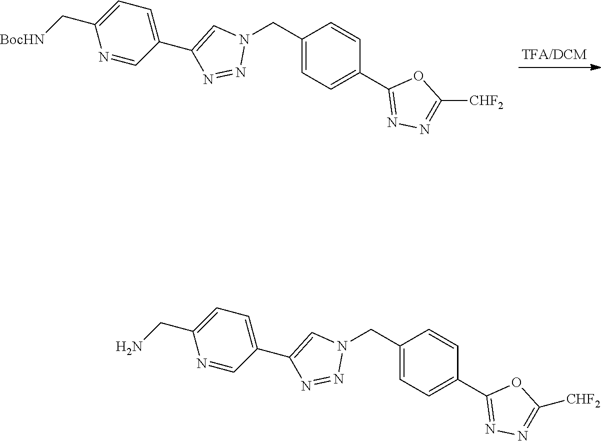 [SMILES: CC(C)(C)OC(=O)NCc1ccc(-c2cn(Cc3ccc(-c4nnc(C(F)F)o4)cc3)nn2)cn1 ; NCc1ccc(-c2cn(Cc3ccc(-c4nnc(C(F)F)o4)cc3)nn2)cn1]

tert-butyl ((5-(1-(4-(5-(difluoromethyl)-1,3,4-oxadiazol-2-yl)benzyl)-1H-1,2,3-triazol-4-yl)pyridin-2-yl)methyl)carbamate (23 mg, 0.043 mmol, 1 equiv.) was dissolved in 2 mL DCM and TFA (20 equiv.) was added. The reaction mixture was stirred at r.t. overnight. The mixture was diluted with DCM and washed with sat. aq. NaHCO₃ and brine. Organic phase was dried over Na₂SO₄, filtered, evaporated. The crude residue was purified by prep-HPLC to obtain target compound (6.1 mg, 0.016 mmol, 30% yield, m/z 384.2 [MH+]).

The following compounds were synthesized according to the same synthetic route:

| Compd. | Structure | m/z [MH+] |
|---|---|---|
| 14 | 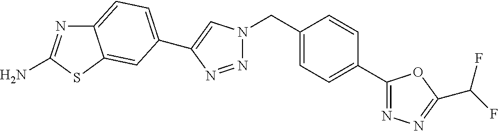 [SMILES: Nc1nc2ccc(-c3cn(Cc4ccc(-c5nnc(C(F)F)o5)cc4)nn3)cc2s1] | 426.92 |
| 46 | 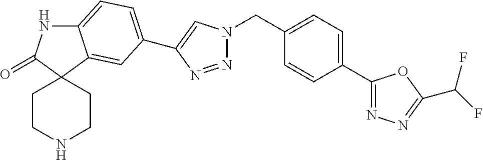 [SMILES: O=C1Nc2ccc(-c3cn(Cc4ccc(-c5nnc(C(F)F)o5)cc4)nn3)cc2C12CCNCC2] | 478.07 |
| 61 | 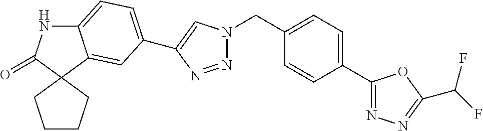 [SMILES: O=C1Nc2ccc(-c3cn(Cc4ccc(-c5nnc(C(F)F)o5)cc4)nn3)cc2C12CCCC2] | 463.5 |
| 84 | 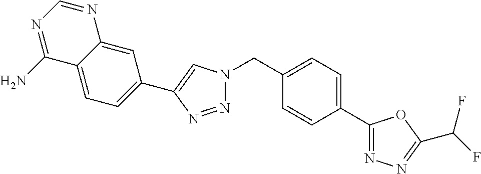 [SMILES: Nc1ncnc2cc(-c3cn(Cc4ccc(-c5nnc(C(F)F)o5)cc4)nn3)ccc12] | 421.3 |
| 101 | 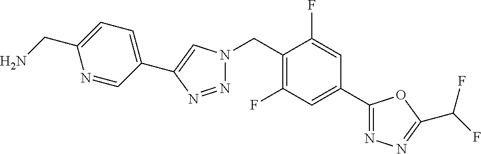 [SMILES: NCc1ccc(-c2cn(Cc3c(F)cc(-c4nnc(C(F)F)o4)cc3F)nn2)cn1] | 420 |
| 132 | 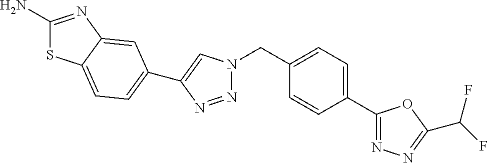 [SMILES: Nc1nc2cc(-c3cn(Cc4ccc(-c5nnc(C(F)F)o5)cc4)nn3)ccc2s1] | 426.4 |
| 161 | 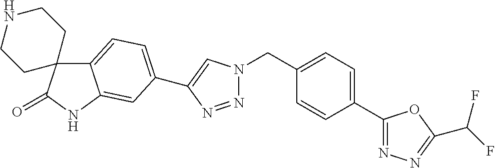 [SMILES: O=C1Nc2cc(-c3cn(Cc4ccc(-c5nnc(C(F)F)o5)cc4)nn3)ccc2C12CCNCC2] | 477.97 |
| 162 | 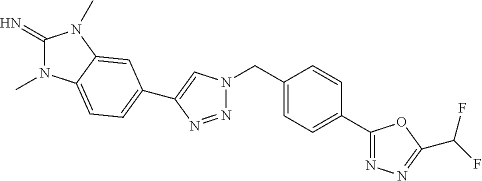 [SMILES: Cn1c(=N)n(C)c2cc(-c3cn(Cc4ccc(-c5nnc(C(F)F)o5)cc4)nn3)ccc21] | 437.7 |
| 166 | 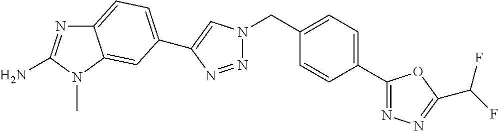 [SMILES: Cn1c(N)nc2ccc(-c3cn(Cc4ccc(-c5nnc(C(F)F)o5)cc4)nn3)cc21] | 423.6 |
| 178 | 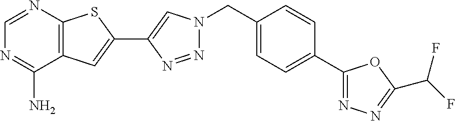 [SMILES: Nc1ncnc2sc(-c3cn(Cc4ccc(-c5nnc(C(F)F)o5)cc4)nn3)cc12] | 427.4 |
| 216 | 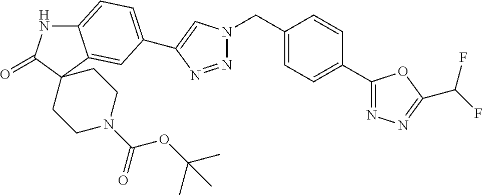 [SMILES: CC(C)(C)OC(=O)N1CCC2(CC1)C(=O)Nc1ccc(-c3cn(Cc4ccc(-c5nnc(C(F)F)o5)cc4)nn3)cc12] | 577.78 |
| 222 | 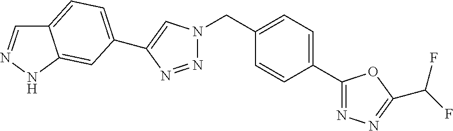 [SMILES: FC(F)c1nnc(-c2ccc(Cn3cc(-c4ccc5cn[nH]c5c4)nn3)cc2)o1] | 393.99 |
| 245 | 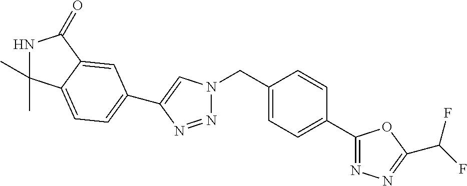 [SMILES: CC1(C)NC(=O)c2cc(-c3cn(Cc4ccc(-c5nnc(C(F)F)o5)cc4)nn3)ccc21] | 437.5 |
| 317 | 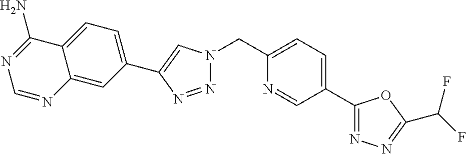 [SMILES: Nc1ncnc2cc(-c3cn(Cc4ccc(-c5nnc(C(F)F)o5)cn4)nn3)ccc12] | 422.02 |
| 324 | 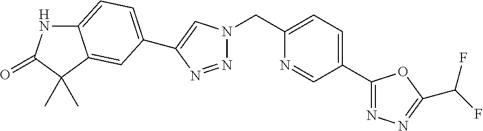 [SMILES: CC1(C)C(=O)Nc2ccc(-c3cn(Cc4ccc(-c5nnc(C(F)F)o5)cn4)nn3)cc21] | 438.01 |
| 325 | 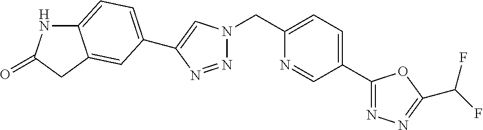 [SMILES: O=C1Cc2cc(-c3cn(Cc4ccc(-c5nnc(C(F)F)o5)cn4)nn3)ccc2N1] | 410 |

### Example 29. Synthesis of 7′-(14 (5-(5-(difluoromethyl)-1,3,4-oxadiazol-2-yl)pyridin-2-yl)methyl)-1H-1,2,3-triazol-4-yl)-1′,4′-dihydro-3′H-spiro[piperidine-4,2′-quinoxalin]-3′-one (Compd. 36)

Step A

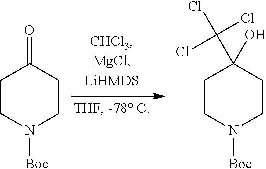 [SMILES: CC(C)(C)OC(=O)N1CCC(=O)CC1 ; CC(C)(C)OC(=O)N1CCC(O)(C(Cl)(Cl)Cl)CC1]

Chloroform (3 equiv.) was added to a mixture of 1-Boc-piperidin-4-one (1 g, 5 mmol, 1 equiv.) and magnesium chloride (3 equiv.) in 15 mL THF. The reaction mixture was cooled in a dry ice/acetone bath. A solution of lithium bis(trimethylsilyl)amide in THF (1.5 equiv., 1M solution) was added over 10 minutes drop-wise, while keeping the internal reaction temperature below −72° C. The reaction was stirred at low temperature overnight and then allowed to warm to rt. The reaction mixture was carefully quenched with water, then partitioned between water and ethyl acetate. The aqueous phase was extracted with ethyl acetate and the combined organic extracts were washed with brine, dried over MgSO₄, filtered and concentrated under reduced pressure. The residue was purified by flash column chromatography (hexane/EtOAc 3:1) affording the product as a white solid (956 mg, 2.99 mmol, 59% yield).

Step B

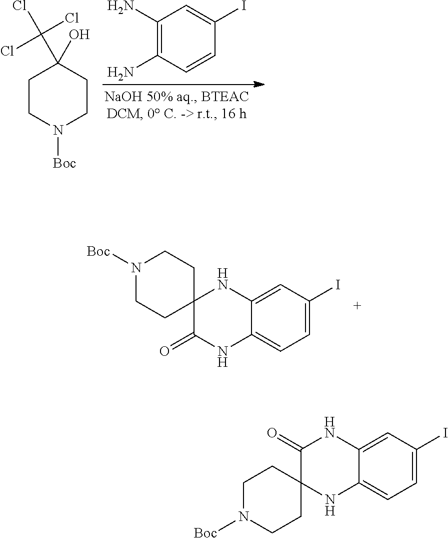

Tert-butyl-4-hydroxy-4-(trichloromethyl)piperidine-1-carboxylate (1.3 equiv.), 4-iodobenzene-1,2-diamine (540 mg, 2.3 mmol, 1 equiv.) and benzyltriethylammonium chloride (0.1 equiv.) were dissolved in DCM under argon. The resulting mixture was cooled to 0° C., and sodium hydroxide (5 equiv., 50% aq. solution) was added dropwise. The reaction mixture was stirred at 0° C. over 1 h and then let to reach r.t. overnight. The reaction mixture was diluted with water (until any solid had dissolved) and the layers were separated. The aqueous layer was extracted with DCM. The combined organic layers were washed with brine, dried over MgSO₄, filtered and concentrated under reduced pressure. The residue was purified by flash column chromatography (hexane/EtOAc 8:2 to 1:1) affording a beige solid (763 mg, 1.67 mmol, 72% yield, mixture of isomers).

Step C

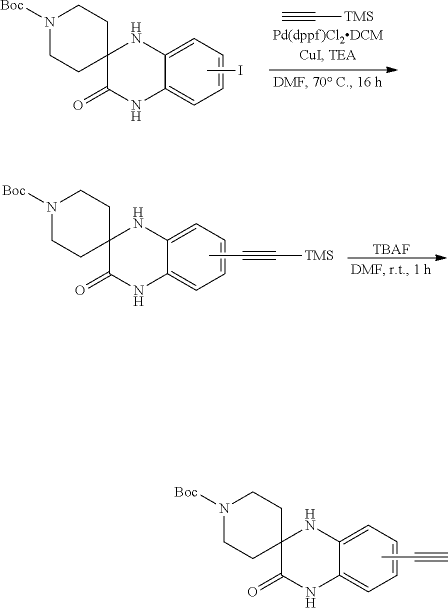

[1,1′-Bis(diphenylphosphino)ferrocene]dichloropalladium (II) DCM complex (0.05 equiv.) and copper(I) iodide (0.1 equiv.) were added to a solution of tert-butyl iodo-3-oxospiro[1,4-dihydroquinoxaline-2,4′-piperidine]-1′-carboxylate (480 mg, 1.08 mmol, 1 equiv., mixture of 6-iodo and 7-iodo isomers) in 5 mL DMF. The mixture was purged with Ar. Ethynyl(trimethyl)silane (1.5 equiv.) and triethylamine (1.1 equiv.) were added. The flask was sealed and the reaction mixture was stirred at 70° C. overnight. Full conversion to the trimethylsilyl protected intermediate was observed. Tetrabutylammonium fluoride solution (1.05 equiv.) was added dropwise, and the resulting mixture was stirred at r.t. over 1 h. The reaction mixture was diluted with water and extracted with EtOAc. The organic phase was washed with water, sat. aq. NaHCO₃ and brine, dried over MgSO₄, filtered and concentrated under reduced pressure. The crude residue was purified by flash column chromatography (hexane/EtOAc 3:1 to 1:1) affording the mixture of products as a yellow solid (414 mg, 1.17 mmol, 69% yield).

Step D

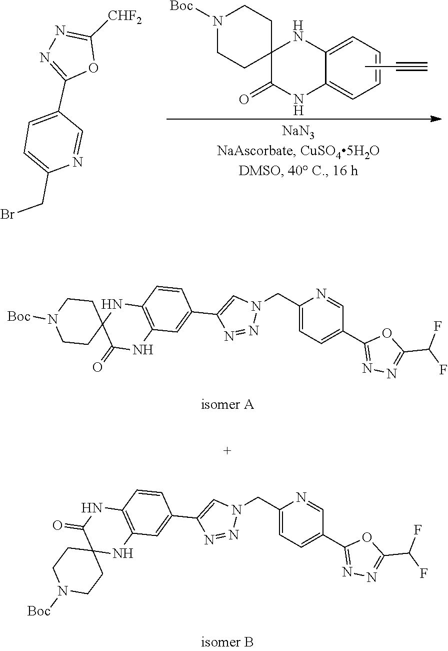

Tert-butyl ethynyl-3′-oxo-3′,4′-dihydro-1′H-spiro[piperidine-4,2′-quinoxaline]-1-carboxylate (200 mg, 0.58 mmol, 1 equiv., mixture of 6′-ethynyl and 7′-ethynyl isomers), 2-[6-(bromomethyl)pyridin-3-yl]-5-(difluoromethyl)-1,3,4-oxadiazole (Intermediate A, 1 equiv.) and sodium azide (1 equiv.) were dissolved in 2.5 mL DMSO. Copper(II) sulfate pentahydrate (0.2 equiv., 0.3 M aqueous solution) and sodium L-ascorbate (0.4 equiv., 0.2 M aqueous solution) were added and the resulting mixture was stirred at r.t. overnight. Full conversion was observed by LC-MS. The reaction mixture was diluted with water and extracted with ethyl acetate. The organic layer was washed with sat. aq. NaHCO₃ and brine, dried over MgSO₄, filtered and concentrated under reduced pressure. The crude yellow solid thus obtained was purified by flash chromatography (hexane/EtOAc 1:1 to 5:95). Separated isomers were afforded as white solids.

Isomer A: 80 mg, 0.13 mmol;

Isomer B: 118 mg, 0.2 mmol.

Step E

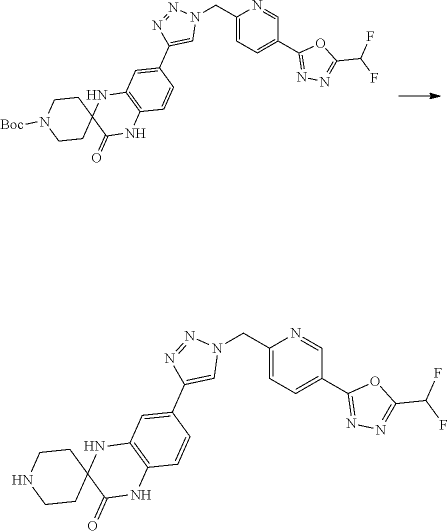 [SMILES: CC(C)(C)OC(=O)N1CCC2(CC1)Nc1cc(-c3cn(Cc4ccc(-c5nnc(C(F)F)o5)cn4)nn3)ccc1NC2=O ; O=C1Nc2ccc(-c3cn(Cc4ccc(-c5nnc(C(F)F)o5)cn4)nn3)cc2NC12CCNCC2]

tert-butyl 7′-(14 (5-(5-(difluoromethyl)-1,3,4-oxadiazol-2-yl)pyridin-2-yl)methyl)-1H-1,2,3-triazol-4-yl)-3′-oxo-3′,4′-dihydro-1′H-spiro[piperidine-4,2′-quinoxaline]-1-carboxylate (isomer B from the previous step, 118 mg, 0.2 mmol, 1 equiv.) was dissolved in 1.5 mL DCE and TFA (12 equiv.) was added. The reaction mixture was stirred at r.t. overnight. Full conversion was detected by LC-MS. The reaction mixture was concentrated under reduced pressure. The residue was dissolved in acetonitrile and concentrated under reduced pressure (3 times). The dark red oily residue obtained was purified by prep-HPLC (formic acid) affording the product as a white solid (8 mg, 0.016, 8% yield). The structure of this compound was confirmed by NOESY. (m/z 494.08 [MH+]).

The following compounds were synthesized following the same synthetic pathway:

| Compd. | Structure | m/z [MH+] |
|---|---|---|
| 41 | 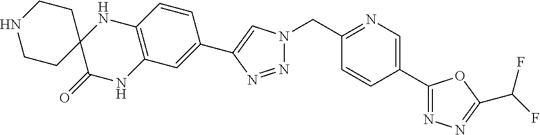 [SMILES: O=C1Nc2cc(-c3cn(Cc4ccc(-c5nnc(C(F)F)o5)cn4)nn3)ccc2NC12CCNCC2] | 494.09 |
| 268 | 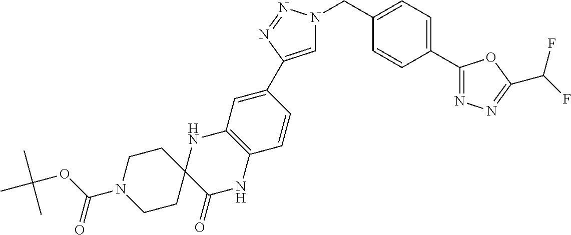 [SMILES: CC(C)(C)OC(=O)N1CCC2(CC1)Nc1cc(-c3cn(Cc4ccc(-c5nnc(C(F)F)o5)cc4)nn3)ccc1NC2=O] | 593.2 |
| 271 | 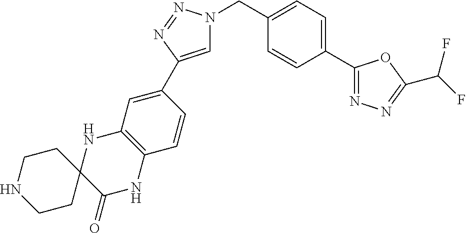 [SMILES: O=C1Nc2ccc(-c3cn(Cc4ccc(-c5nnc(C(F)F)o5)cc4)nn3)cc2NC12CCNCC2] | 493.18 |
| 277 | 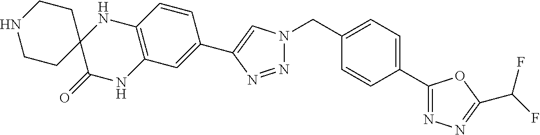 [SMILES: O=C1Nc2cc(-c3cn(Cc4ccc(-c5nnc(C(F)F)o5)cc4)nn3)ccc2NC12CCNCC2] | 493.15 |
| 279 | 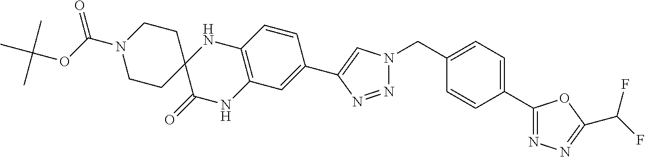 [SMILES: CC(C)(C)OC(=O)N1CCC2(CC1)Nc1ccc(-c3cn(Cc4ccc(-c5nnc(C(F)F)o5)cc4)nn3)cc1NC2=O] | 592.98 |

### Example 30. Synthesis of 5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one (enantiomer B) (Compd. 274), tert-butyl 5-[1-({5-[5-(difluoromethyl)-1,3,4-oxadiazol-2-yl]pyridin-2-yl}methyl)-1H-1,2,3-triazol-4-yl]-2-oxo-1,2-dihydrospiro[indole-3,3′-pyrrolidine]-1′-carboxylate (Enantiomer A) (Compd. 265) and tert-butyl 5-[1-({5-[5-(difluoromethyl)-1,3,4-oxadiazol-2-yl]pyridin-2-yl}methyl)-1H-1,2,3-triazol-4-yl]-2-oxo-1,2-dihydrospiro[indole-3,3′-pyrrolidine]-1′-carboxylate (Enantiomer B) (Compd. 266)

Step A

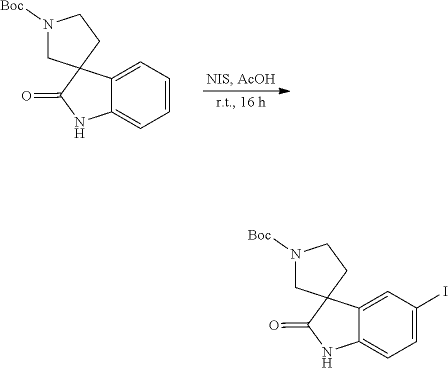 [SMILES: CC(C)(C)OC(=O)N1CCC2(C1)C(=O)Nc1ccccc12 ; CC(C)(C)OC(=O)N1CCC2(C1)C(=O)Nc1ccc(I)cc12]

A mixture of tert-butyl 2-oxospiro[1H-indole-3,3′-pyrrolidine]-t-carboxylate (1.15 g, 4 mmol, 1 equiv.) and N-iodosuccinimide (1.2 equiv.) in acetic acid (7 mL) was stirred under argon at r.t. overnight. Conversion was monitored by LC-MS. Water was added to the reaction mixture and precipitation occurred. The product was extracted with ethyl acetate and the organic layer was washed with a 10% aq. Na₂S₂O₃ and brine. The organic layer was dried over MgSO₄, filtered and concentrated under reduced pressure affording a dense yellow oil (1.66 g, 4 mmol, 100% yield) which was used directly in the next step.

Step B

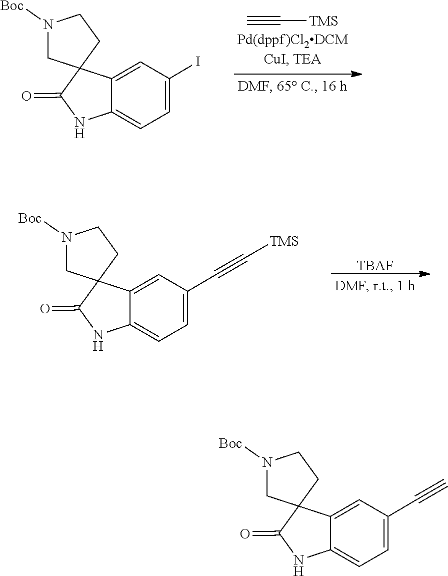

[1,1′-Bis(diphenylphosphino)ferrocene]dichloropalladium(II) DCM complex (0.05 equiv.) and copper(I) iodide (0.1 equiv.) were added to a solution of tert-butyl 5-iodo-2-oxospiro[indoline-3,3′-pyrrolidine]-1′-carboxylate (1.66 g, 4 mmol, 1 equiv.) in 8 mL DMF. The mixture was purged with Ar. Ethynyl(trimethyl)silane (1.5 equiv.) and triethylamine (1.1 equiv.) were added. The flask was sealed and the reaction mixture was stirred at 65° C. overnight. Full conversion to the trimethylsilyl protected intermediate was observed.

Tetrabutylammonium fluoride solution (1.05 equiv.) was added dropwise, and the resulting mixture was stirred at r.t. over 1 h. The reaction mixture was diluted with water and extracted with EtOAc. The organic phase was washed with water, sat. aq. NaHCO₃ and brine, dried over MgSO₄, filtered and concentrated under reduced pressure. The crude residue was purified by flash column chromatography (hexane/EtOAc 3:1 to 1:1) affording the product as a yellow solid (504 mg, 1.61 mmol, 40% yield). Step C

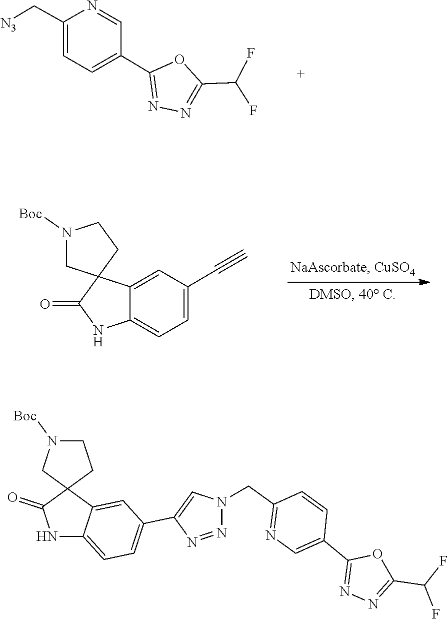 [SMILES: [N-]=[N+]=NCc1ccc(-c2nnc(C(F)F)o2)cn1 ; C#Cc1ccc2c(c1)C1(CCN(C(=O)OC(C)(C)C)C1)C(=O)N2 ; CC(C)(C)OC(=O)N1CCC2(C1)C(=O)Nc1ccc(-c3cn(Cc4ccc(-c5nnc(C(F)F)o5)cn4)nn3)cc12]

Copper(II) sulfate pentahydrate (0.2 equiv., 0.12 M aqueous solution) and sodium L-ascorbate (0.4 equiv., 0.25 M aqueous solution) were added to a solution of 2-(6-(azidomethyl)pyridin-3-yl)-5-(difluoromethyl)-1,3,4-oxadiazole (Intermediate F, 153 mg, 0.61 mmol, 1 equiv.) and tert-butyl 5-ethynyl-2-oxospiro[indoline-3,3′-pyrrolidine]-1′-carboxylate (190 mg, 0.61 mmol, 1 equiv.) in 2 mL DMSO. The reaction mixture was stirred at r.t. overnight. Full conversion of the starting material was detected by LC-MS. The reaction mixture was diluted with water and extracted with EtOAc. The organic layer was washed with brine, dried over MgSO₄, filtered and concentrated under reduced pressure. The residue was purified by flash column chromatography (hexane/EtOAc 1:2 to 1:9) affording the desired product as a white solid (240 mg, 0.42 mmol, 70% yield).

Step D

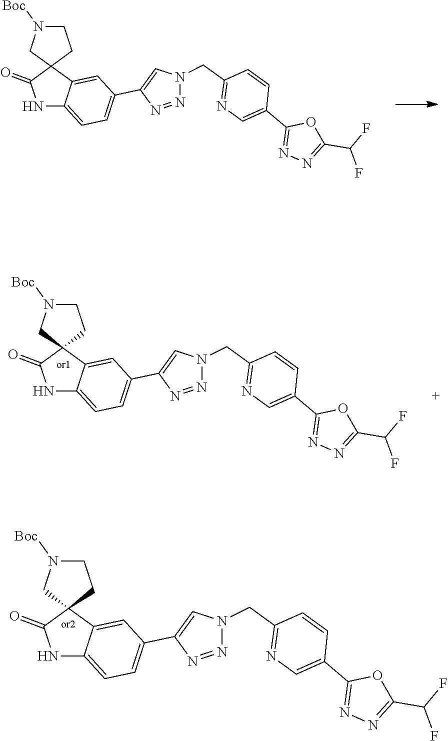 [SMILES: CC(C)(C)OC(=O)N1CCC2(C1)C(=O)Nc1ccc(-c3cn(Cc4ccc(-c5nnc(C(F)F)o5)cn4)nn3)cc12 ; CC(C)(C)OC(=O)N1CC[C@@]2(C1)C(=O)Nc1ccc(-c3cn(Cc4ccc(-c5nnc(C(F)F)o5)cn4)nn3)cc12 ; CC(C)(C)OC(=O)N1CC[C@]2(C1)C(=O)Nc1ccc(-c3cn(Cc4ccc(-c5nnc(C(F)F)o5)cn4)nn3)cc12]

tert-butyl 5-(1-((5-(5-(difluoromethyl)-1,3,4-oxadiazol-2-yl)pyridin-2-yl)methyl)-1H-1,2,3-triazol-4-yl)-2-oxospiro[indoline-3,3′-pyrrolidine]-1′-carboxylate (123 mg, 0.21 mmol) was dissolved to 20 mg/mL in MeOH and was then purified by SFC. Combined fractions of each of the enantiomers were then evaporated to dryness under reduced pressure. The resultant solids were then dried in a vacuum oven at 35° C. and 5 mbar until constant weight to afford pure enantiomers as colourless glasses.

(enantiomer A): compd. 256 (49 mg, 0.087 mmol, 99.4% ee, m/z 565.20 [MH+])

(enantiomer B): compd. 266 (50 mg, 0.087 mmol, 98.2% ee, m/z 565.23 [MH+])

Step E

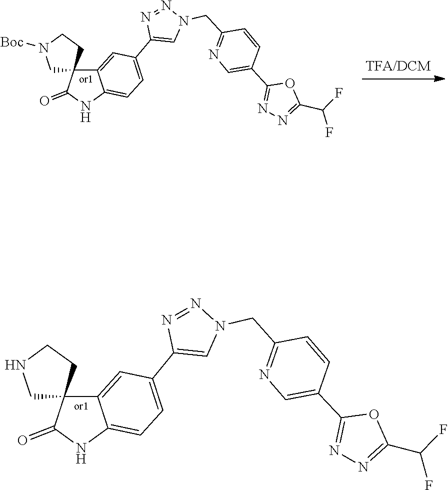 [SMILES: CC(C)(C)OC(=O)N1CC[C@]2(C1)C(=O)Nc1ccc(-c3cn(Cc4ccc(-c5nnc(C(F)F)o5)cn4)nn3)cc12 ; O=C1Nc2ccc(-c3cn(Cc4ccc(-c5nnc(C(F)F)o5)cn4)nn3)cc2[C@]12CCNC2]

Tert-butyl 5-(1-((5-(5-(difluoromethyl)-1,3,4-oxadiazol-2-yl)pyridin-2-yl)methyl)-1H-1,2,3-triazol-4-yl)-2-oxospiro[indoline-3,3′-pyrrolidine]-1′-carboxylate (enantiomer B, 50 mg, 0.089 mmol, 1 equiv.) was dissolved in 1 mL DCE and TFA (12 equiv.) was added. The reaction mixture was stirred at r.t. over 4 h. The mixture was then concentrated under reduced pressure, and the residue thus obtained was dissolved in acetonitrile and concentrated under reduced pressure (3×). The crude residue was purified by prep-HPLC (formic acid) affording the product as a white solid (8 mg, 0.017 mmol, 19% yield, m/z 465.01 [MH+]).

The following compounds were prepared according to the same procedure:

| Compd. | Structure | m/z [MH+] |
|---|---|---|
| 28 | 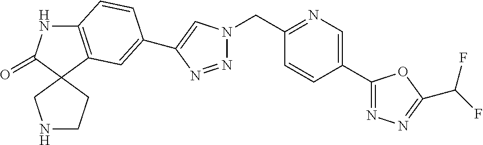 [SMILES: O=C1Nc2ccc(-c3cn(Cc4ccc(-c5nnc(C(F)F)o5)cn4)nn3)cc2C12CCNC2] | 465.02 |
| 49 | 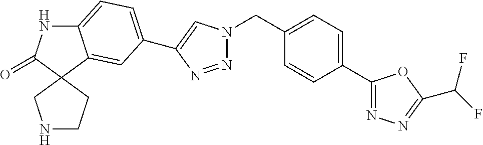 [SMILES: O=C1Nc2ccc(-c3cn(Cc4ccc(-c5nnc(C(F)F)o5)cc4)nn3)cc2C12CCNC2] | 463.94 |
| 177 | 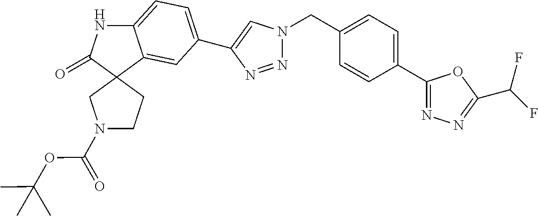 [SMILES: CC(C)(C)OC(=O)N1CCC2(C1)C(=O)Nc1ccc(-c3cn(Cc4ccc(-c5nnc(C(F)F)o5)cc4)nn3)cc12] | 564.02 |
| 273 | 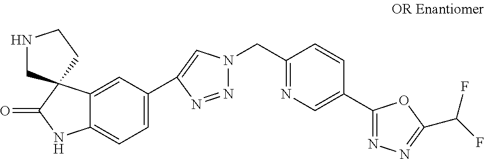 [SMILES: O=C1Nc2ccc(-c3cn(Cc4ccc(-c5nnc(C(F)F)o5)cn4)nn3)cc2[C@@]12CCNC2] | 465.1 |

### Example 31. Synthesis of 5-(1-((5-(5-(difluoromethyl)-1,3,4-oxadiazol-2-yl)pyridin-2-yl)methyl)-1H-1,2,3-triazol-4-yl)-N-ethyl-1H-benzo[d]imidazol-2-amine (Compd. 18)

Step A

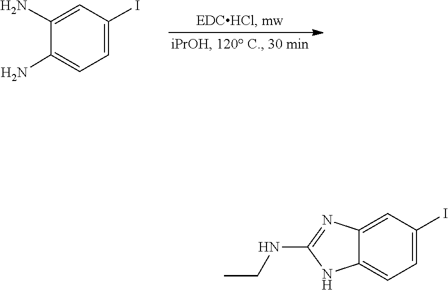

4-iodobenzene-1,2-diamine (600 mg, 2.56 mmol, 1 equiv.) and 1-(3-Dimethylaminopropyl)-3-ethylcarbodiimide hydrochloride (1.1 equiv.) were dissolved in 15 mL isopropanol and stirred for 30 min at 120° C. under microwave irradiation. The reaction mixture was diluted with EtOAc and washed with water (2×) and brine. Organic phase was dried over Na₂SO₄, filtered and concentrated under reduced pressure to give a crude product (730 mg, 2.54 mmol, 99% yield).

Next step was set on crude product.

Step B

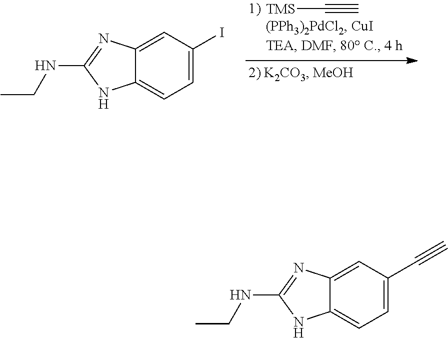

N-ethyl-5-iodo-1H-benzo[d]imidazol-2-amine (730 mg, 2.54 mmol, 1 equiv.) and ethynyl(trimethyl)silane (1.5 equiv.) were dissolved in a triethylamine (2 equiv.) solution in DMF (8 mL). The mixture was degassed with Ar, copper iodide (0.1 equiv.) and [1,1′-Bis(diphenylphosphino)ferrocene]dichloropalladium(II) DCM complex (0.1 equiv.) were added. The reaction mixture was degassed again, heated to 80° C. and stirred overnight. Full conversion to TMS protected intermediate was observed by LC-MS. The reaction mixture was diluted with EtOAc and evaporated in presence of silica-gel (15 g). The intermediate product was purified by flash column chromatography (0-5% MeOH/DCM, dry load).

Purified intermediate was dissolved in MeOH, and potassium carbonate (2 equiv.) was added. The reaction mixture was stirred at r.t. over 1 h. MeOH was then evaporated, the residue was suspended in EtOAc and filtered. The desired product was in the filtrate, which was concentrated to dryness to give product (430 mg, 2.32 mmol, 91% yield).

Step C

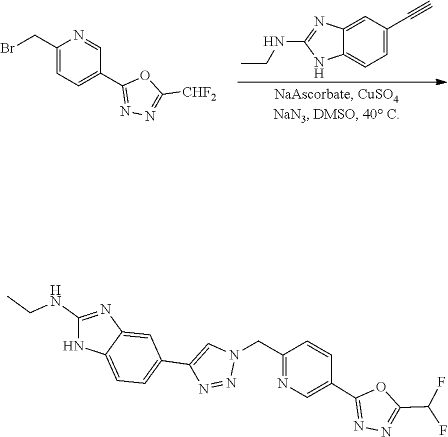 [SMILES: FC(F)c1nnc(-c2ccc(CBr)nc2)o1 ; C#Cc1ccc2[nH]c(NCC)nc2c1 ; CCNc1nc2cc(-c3cn(Cc4ccc(-c5nnc(C(F)F)o5)cn4)nn3)ccc2[nH]1]

N-ethyl-5-ethynyl-1H-benzimidazol-2-amine (80 mg, 0.432 mmol, 1 equiv.), 2-[6-(bromomethyl)pyridin-3-yl]-5-(difluoromethyl)-1,3,4-oxadiazole (Intermediate A, 125 mg, 0.432 mmol, 1 equiv.) and azide (1 equiv.) were dissolved in DMSO. Copper(II) sulfate pentahydrate (0.2 equiv., 0.12 M aqueous solution) and sodium L-ascorbate (0.4 equiv., 0.25 M aqueous solution) were added, and the mixture was stirred at r.t. overnight. The reaction mixture was submitted to prep-HPLC (C-18 neutral conditions) without any workup, obtaining 18.6 mg of pure product (0.042 mmol, 10% yield, m/z 438.12 [MH+])

The following compound was prepared according to the same procedure:

| Compd. | Structure | m/z [MH+] |
|---|---|---|
| 60 | 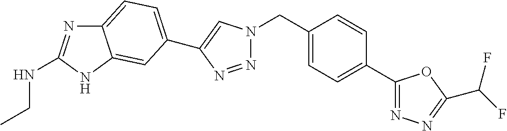 [SMILES: CCNc1nc2ccc(-c3cn(Cc4ccc(-c5nnc(C(F)F)o5)cc4)nn3)cc2[nH]1] | 437.09 |

### Example 32. Synthesis of 2-amino-N-(3-(1-(4-(5-(difluoromethyl)-1,3,4-oxadiazol-2-yl)-2,6-difluorobenzyl)-1H-1,2,3-triazol-4-yl)phenyl)acetamide (Compd. 144) and tert-butyl (2-((3-(1-(4-(5-(difluoromethyl)-1,3,4-oxadiazol-2-yl)-2,6-difluorobenzyl)-1H-1,2,3-triazol-4-yl)phenyl)amino)-2-oxoethyl)carbamate (Compd. 172)

Step A

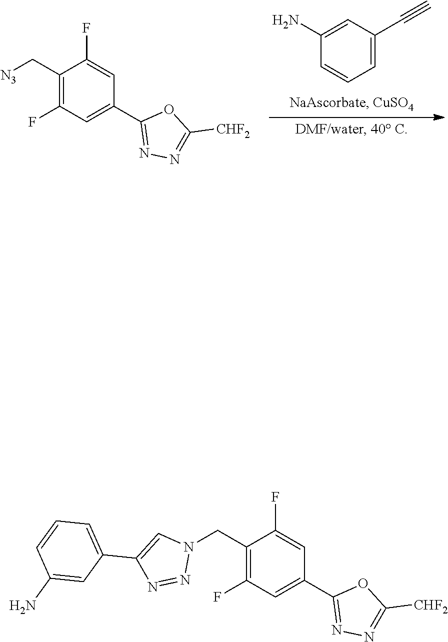

Copper(II) sulfate pentahydrate (0.1 equiv., 0.05 M aqueous solution) and sodium L-ascorbate (0.5 equiv., 0.25 M aqueous solution) were added to a solution of 2-(4-(azidomethyl)-2,3-difluorophenyl)-5-(difluoromethyl)-1,3,4-oxadiazole (Intermediate I, 1.1 equiv.) and 3-aminophenylacetylene (145 mg, 1.24 mmol, 1 equiv.) in 5 mL DMF. The reaction mixture was stirred at 35° C. overnight. Full conversion of the starting material was detected by LC-MS.

The reaction mixture was diluted with EtOAc and washed with water. Water phase was extracted with EtOAc (3×). Combined organic layers were dried over MgSO₄ and concentrated by rotary evaporation to give a crude product as a solution in DMF, which was used in the next step.

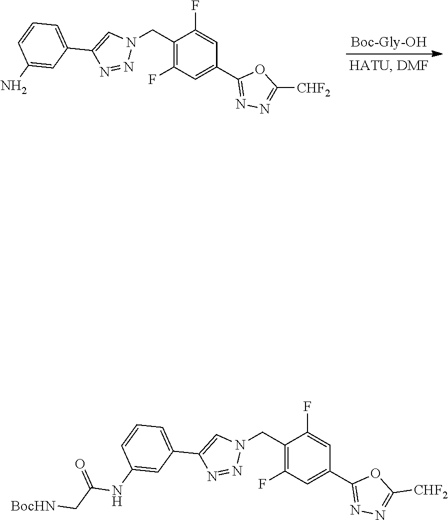 [SMILES: Nc1cccc(-c2cn(Cc3c(F)cc(-c4nnc(C(F)F)o4)cc3F)nn2)c1 ; "Boc-Gly-OH" ; CC(C)(C)OC(=O)NCC(=O)Nc1cccc(-c2cn(Cc3c(F)cc(-c4nnc(C(F)F)o4)cc3F)nn2)c1]

Boc-glycine (3 equiv.) and HATU (3 equiv.) were stirred for 30 min in 2.5 mL DMF. Then 3-[1-[[4-[5-(difluoromethyl)-1,3,4-oxadiazol-2-yl]-2,6-difluorophenyl]methyl]triazol-4-yl]aniline (0.618 mmol, 1 equiv., 0.25 M solution in DMF) was added. The reaction mixture was stirred at r.t. overnight. The reaction mixture was diluted with EtOAc and washed with water. Water phase was extracted with EtOAc (3×). Combined organic phases were dried over MgSO₄ and evaporated to give a crude product, which was purified by flash column chromatography (0-2% MeOH/DCM) (compd. 172, 87 mg, 0.155, 25% yield compd., m/z 561.68 [MH+])

Step C

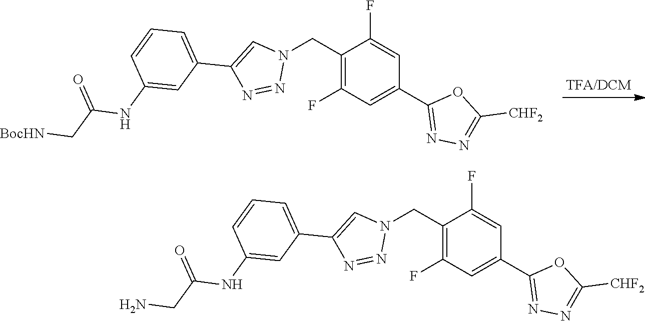

tert-butyl N-[2-[3-[1-[[4-[5-(difluoromethyl)-1,3,4-oxadiazol-2-yl]-2,6-difluorophenyl]methyl]triazol-4-yl]anilino]-2-oxoethyl]carbamate (80 mg, 0.142 mmol 1 equiv.) was dissolved in 6 mL DCM, then TFA (15 equiv.) was added. The reaction mixture was stirred at r.t. overnight.

The reaction mixture was diluted with DCM and washed with sat. aq. NaHCO₃. Organic phase was dried over Na₂SO₄ and evaporated to give a crude product, which was purified by pTLC (4-6% MeOH/DCM) (17 mg, 0.036 mmol, 25% yield, m/z 461.95 [MH+])

### Example 33. Synthesis of 5-((4-(1-(4-(5-(difluoromethyl)-1,3,4-oxadiazol-2-yl)-2,6-difluorobenzyl)-1H-1,2,3-triazol-4-yl)benzyl)amino)-2-methoxynicotinamide (Compd. 154)

Step A

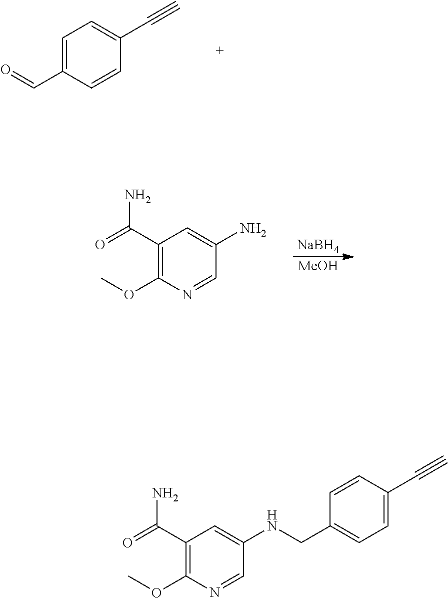

4-ethynylenzaldehyde (60 mg, 0.46 mmol, 1 equiv.) and 5-amino-2-methoxypyridine-3-carboxamide (1.1 equiv.) were dissolved in 20 mL MeOH. The mixture was stirred overnight, until full conversion to the corresponding imine was detected by LC-MS. Sodium borohydride (12 equiv.) was added in portions and the reaction mixture was stirred overnight. The reaction mixture was evaporated, dissolved in EtOAc and washed with water. Water phase was extracted with EtOAc (3×). Combined organic phases were dried and evaporated to give a crude product which was purified by pTLC (0-4% MeOH/DCM) (28 mg, 0.1 mmol, 22% yield).

Step B

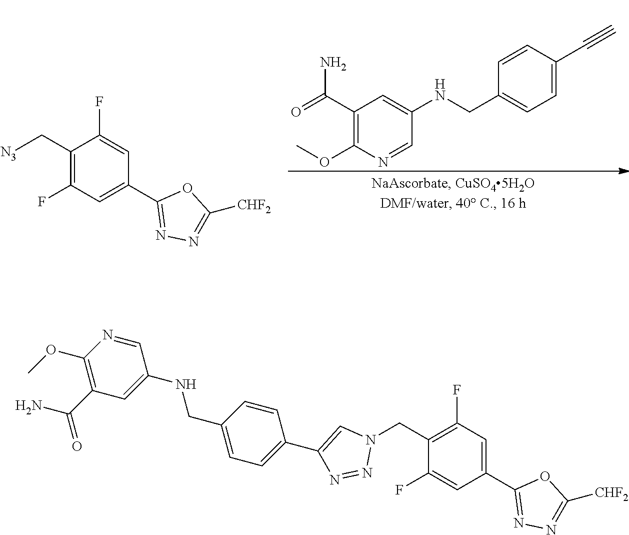

Copper(II) sulfate pentahydrate (0.1 equiv., 0.01 M aqueous solution) and sodium L-ascorbate (0.5 equiv., 0.05 M aqueous solution) were added to a solution of 2-(4-(azidomethyl)-2,3-difluorophenyl)-5-(difluoromethyl)-1,3,4-oxadiazole (Intermediate I, 1.1 equiv.) and 5-((4-ethynylbenzyl)amino)-2-methoxynicotinamide (28 mg, 0.1 mmol, 1 equiv.) in 2 mL DMF. The reaction mixture was agitated at 40° C. overnight. Full conversion of the starting material was detected by LC-MS. The reaction mixture was diluted with EtOAc and washed with water. Aqueous phase was extracted with EtOAc (3×). Organic phases were combined, dried over Na₂SO₄, filtered and evaporated to give a crude product. Purification by pTLC (2% MeOH/DCM) and then by prep-HPLC (0.1% FA/ACN/water C-18) gave, after evaporation of fractions, 9 mg of target compound (0.02 mmol, 17% yield) as an off-white solid (m/z 569.20 [MH+]).

### Example 34. Synthesis of 5-(1-((5-(5-(difluoromethyl)-1,3,4-oxadiazol-2-yl)pyridin-2-yl)methyl)-1H-1,2,3-triazol-4-yl)-1-methyl-1H-benzo[d]imidazol-2-amine (Compd. 17)

Step A

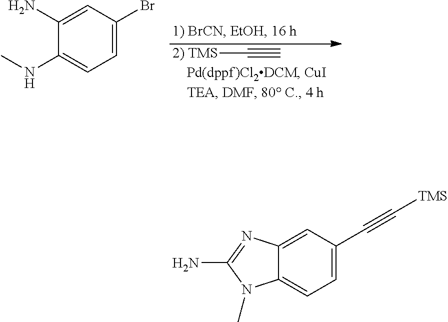

4-bromo-1-N-methylbenzene-1,2-diamine (500 mg, 2.49 mmol, 1 equiv.) was dissolved in 10 mL EtOH. Cyanogen bromide (1.1 equiv.) was added, and the resulting mixture was stirred at r.t. overnight. Full conversion was observed by LC-MS. The reaction mixture was concentrated and dried under reduced pressure.

The crude intermediate and ethynyl(trimethyl)silane were dissolved in a triethylamine (1.6 equiv.) solution in DMF (7 mL) and the resulting mixture was degassed with Ar. Copper iodide (0.1 equiv.) and [1,1′-bis(diphenylphosphino)ferrocene]dichloropalladium(II) dichloromethane complex (0.1 equiv.) were added. The reaction mixture was degassed again, and then stirred at 80° C. over 4 h. According to LC-MS the desired product was mainly formed. The reaction mixture was diluted with EtOAc and evaporated in presence of 40 g of silica gel. Purification by flash column chromatography (0-5% MeOH/DCM, dry load) gave 137 mg of product (0.53 mmol, 22% yield).

Step B

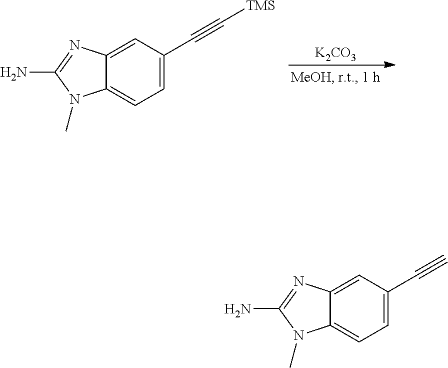

1-methyl-5-((trimethylsilypethynyl)-1H-benzo[d]imidazol-2-amine (137 mg, 0.53 mmol, 1 equiv.) was dissolved in 5 mL MeOH, and potassium carbonate (2 equiv.) was added. The reaction mixture was stirred at r.t. over 1 h. Volatiles were removed by evaporation, the residue was suspended in EtOAc and filtered. Filtrate was evaporated to give product (76 mg, 0.44 mmol, 83% yield).

Step C

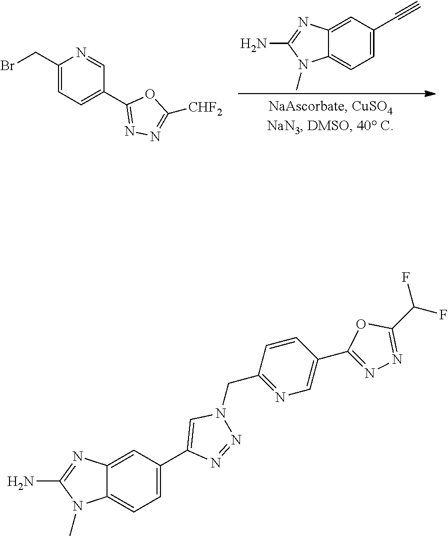 [SMILES: FC(F)c1nnc(-c2ccc(CBr)nc2)o1 ; C#Cc1ccc2c(c1)nc(N)n2C ; Cn1c(N)nc2cc(-c3cn(Cc4ccc(-c5nnc(C(F)F)o5)cn4)nn3)ccc21]

5-ethynyl-1-methyl-1H-benzo[d]imidazol-2-amine (76 mg, 0.44 mmol, 1 equiv.), 2-[6-(bromomethyl)pyridin-3-yl]-5-(difluoromethyl)-1,3,4-oxadiazole (Intermediate A, 129 mg, 0.44 mmol, 1 equiv.) and sodium azide (1 equiv.) were dissolved in 2 mL DMSO. Copper(II) sulfate pentahydrate (0.2 equiv., 0.09 M aqueous solution) and sodium L-ascorbate (0.4 equiv., 0.18 M aqueous solution) were added, and the mixture was stirred at r.t. overnight. The reaction mixture was submitted to prep-HPLC (C-18 neutral conditions) without any workup, obtaining 36 mg of pure product (0.085 mmol, 19% yield, m/z 423.95 [MH+]).

The following compounds were prepared according to the same procedure:

| Compd. | Structure | m/z [MH+] |
|---|---|---|
| 1 | 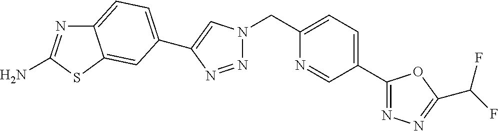 [SMILES: Nc1nc2ccc(-c3cn(Cc4ccc(-c5nnc(C(F)F)o5)cn4)nn3)cc2s1] | 427.21 |
| 3 | 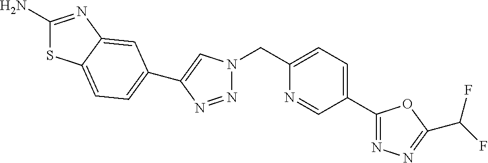 [SMILES: Nc1nc2cc(-c3cn(Cc4ccc(-c5nnc(C(F)F)o5)cn4)nn3)ccc2s1] | 427.4 |
| 19 | 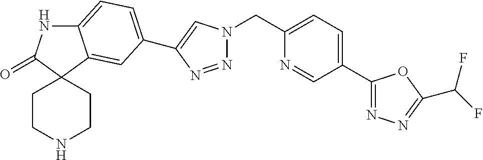 [SMILES: O=C1Nc2ccc(-c3cn(Cc4ccc(-c5nnc(C(F)F)o5)cn4)nn3)cc2C12CCNCC2] | 479.02 |
| 23 | 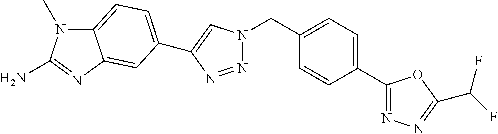 [SMILES: Cn1c(N)nc2cc(-c3cn(Cc4ccc(-c5nnc(C(F)F)o5)cc4)nn3)ccc21] | 423.5 |
| 26 | 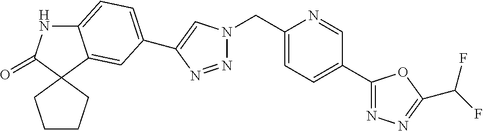 [SMILES: O=C1Nc2ccc(-c3cn(Cc4ccc(-c5nnc(C(F)F)o5)cn4)nn3)cc2C12CCCC2] | 464.09 |
| 123 | 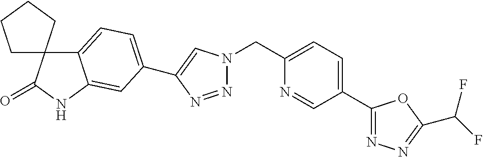 [SMILES: O=C1Nc2cc(-c3cn(Cc4ccc(-c5nnc(C(F)F)o5)cn4)nn3)ccc2C12CCCC2] | 464.4 |
| 339 | 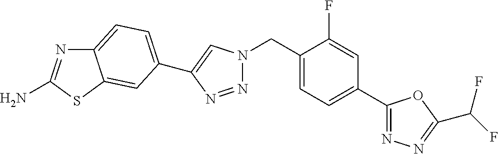 [SMILES: Nc1nc2ccc(-c3cn(Cc4ccc(-c5nnc(C(F)F)o5)cc4F)nn3)cc2s1] | 444.03 |
| 340 | 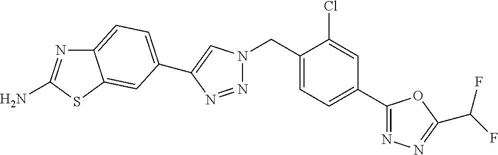 [SMILES: Nc1nc2ccc(-c3cn(Cc4ccc(-c5nnc(C(F)F)o5)cc4Cl)nn3)cc2s1] | 460.11 |
| 341 | 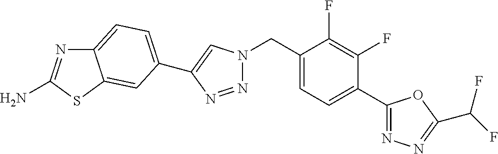 [SMILES: Nc1nc2ccc(-c3cn(Cc4ccc(-c5nnc(C(F)F)o5)c(F)c4F)nn3)cc2s1] | 462.34 |
| 342 | 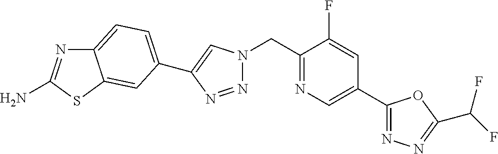 [SMILES: Nc1nc2ccc(-c3cn(Cc4ncc(-c5nnc(C(F)F)o5)cc4F)nn3)cc2s1] | 445.15 |
| 343 | 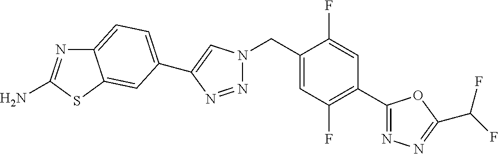 [SMILES: Nc1nc2ccc(-c3cn(Cc4cc(F)c(-c5nnc(C(F)F)o5)cc4F)nn3)cc2s1] | 462.13 |
| 344 | 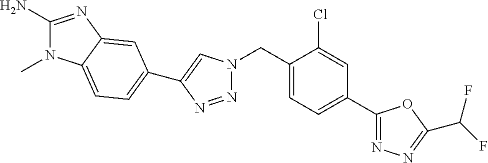 [SMILES: Cn1c(N)nc2cc(-c3cn(Cc4ccc(-c5nnc(C(F)F)o5)cc4Cl)nn3)ccc21] | 457.11 |
| 345 | 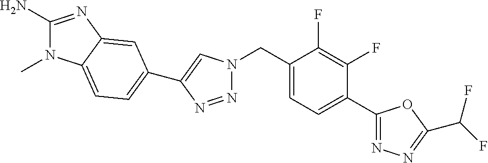 [SMILES: Cn1c(N)nc2cc(-c3cn(Cc4ccc(-c5nnc(C(F)F)o5)c(F)c4F)nn3)ccc21] | 459.11 |
| 346 | 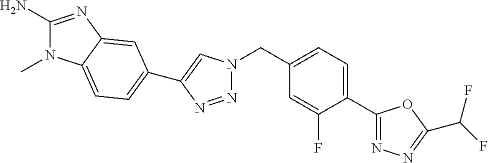 [SMILES: Cn1c(N)nc2cc(-c3cn(Cc4ccc(-c5nnc(C(F)F)o5)c(F)c4)nn3)ccc21] | 441.19 |
| 347 | 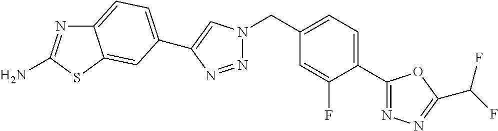 [SMILES: Nc1nc2ccc(-c3cn(Cc4ccc(-c5nnc(C(F)F)o5)c(F)c4)nn3)cc2s1] | 444.15 |
| 349 | 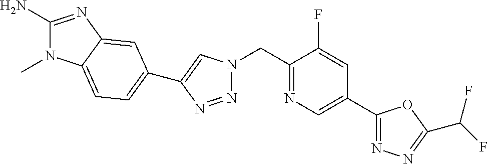 [SMILES: Cn1c(N)nc2cc(-c3cn(Cc4ncc(-c5nnc(C(F)F)o5)cc4F)nn3)ccc21] | 442.43 |
| 350 | 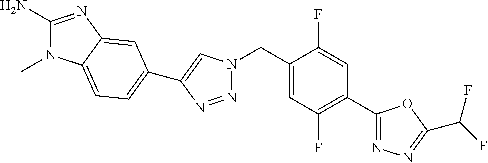 [SMILES: Cn1c(N)nc2cc(-c3cn(Cc4cc(F)c(-c5nnc(C(F)F)o5)cc4F)nn3)ccc21] | 459.07 |
| 351 | 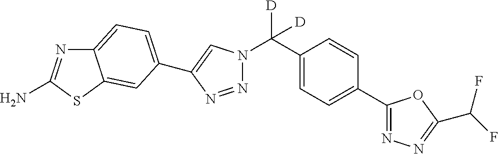 [SMILES: [2H]C([2H])(c1ccc(-c2nnc(C(F)F)o2)cc1)n1cc(-c2ccc3nc(N)sc3c2)nn1] | 428.3 |
| 376 | 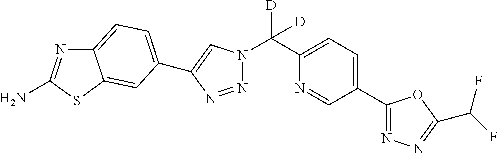 [SMILES: [2H]C([2H])(c1ccc(-c2nnc(C(F)F)o2)cn1)n1cc(-c2ccc3nc(N)sc3c2)nn1] | 429.16 |
| 382 | 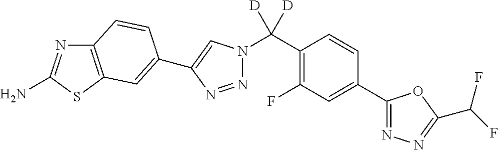 [SMILES: [2H]C([2H])(c1ccc(-c2nnc(C(F)F)o2)cc1F)n1cc(-c2ccc3nc(N)sc3c2)nn1] | 446.13 |
| 378 | 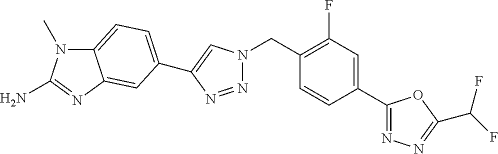 [SMILES: Cn1c(N)nc2cc(-c3cn(Cc4ccc(-c5nnc(C(F)F)o5)cc4F)nn3)ccc21] | 441.18 |
| 384 | 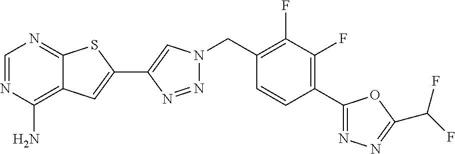 [SMILES: Nc1ncnc2sc(-c3cn(Cc4ccc(-c5nnc(C(F)F)o5)c(F)c4F)nn3)cc12] | 463.85 |
| 386 | 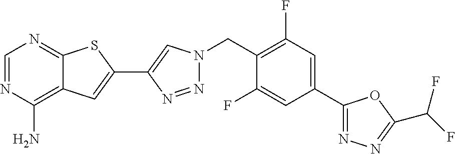 [SMILES: Nc1ncnc2sc(-c3cn(Cc4c(F)cc(-c5nnc(C(F)F)o5)cc4F)nn3)cc12] | 462.84 |
| 415 | 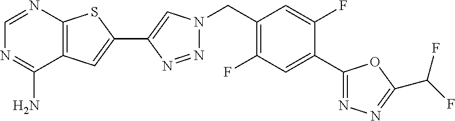 [SMILES: Nc1ncnc2sc(-c3cn(Cc4cc(F)c(-c5nnc(C(F)F)o5)cc4F)nn3)cc12] | 463.85 |
| 416 | 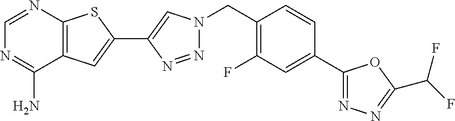 [SMILES: Nc1ncnc2sc(-c3cn(Cc4ccc(-c5nnc(C(F)F)o5)cc4F)nn3)cc12] | 444.86 |
| 387 | 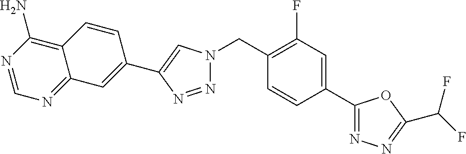 [SMILES: Nc1ncnc2cc(-c3cn(Cc4ccc(-c5nnc(C(F)F)o5)cc4F)nn3)ccc12] | 438.92 |
| 399 | 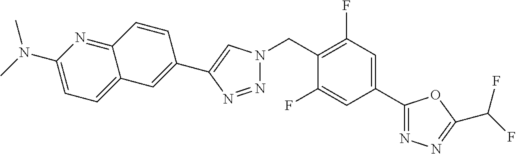 [SMILES: CN(C)c1ccc2cc(-c3cn(Cc4c(F)cc(-c5nnc(C(F)F)o5)cc4F)nn3)ccc2n1] | 485.17 |
| 402 | 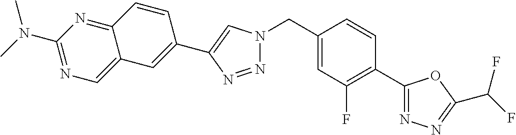 [SMILES: CN(C)c1ncc2cc(-c3cn(Cc4ccc(-c5nnc(C(F)F)o5)c(F)c4)nn3)ccc2n1] | 467.18 |
| 404 | 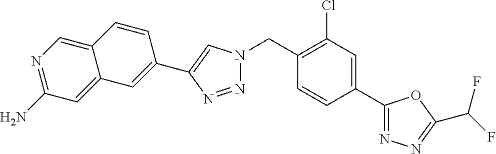 [SMILES: Nc1cc2cc(-c3cn(Cc4ccc(-c5nnc(C(F)F)o5)cc4Cl)nn3)ccc2cn1] | 454.15 |
| 405 | 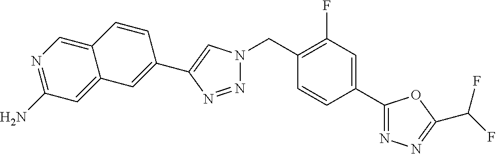 [SMILES: Nc1cc2cc(-c3cn(Cc4ccc(-c5nnc(C(F)F)o5)cc4F)nn3)ccc2cn1] | 438.16 |
| 406 | 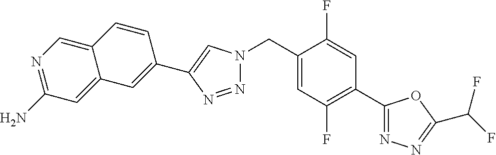 [SMILES: Nc1cc2cc(-c3cn(Cc4cc(F)c(-c5nnc(C(F)F)o5)cc4F)nn3)ccc2cn1] | 456.12 |
| 417 | 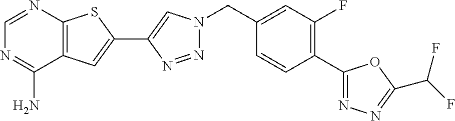 [SMILES: Nc1ncnc2sc(-c3cn(Cc4ccc(-c5nnc(C(F)F)o5)c(F)c4)nn3)cc12] | 445.12 |
| 418 | 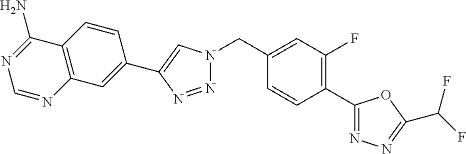 [SMILES: Nc1ncnc2cc(-c3cn(Cc4ccc(-c5nnc(C(F)F)o5)c(F)c4)nn3)ccc12] | 439.19 |
| 433 | 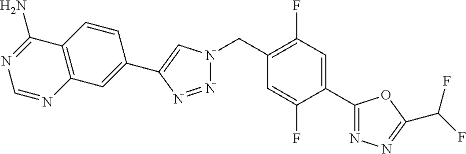 [SMILES: Nc1ncnc2cc(-c3cn(Cc4cc(F)c(-c5nnc(C(F)F)o5)cc4F)nn3)ccc12] | 457.16 |
| 434 | 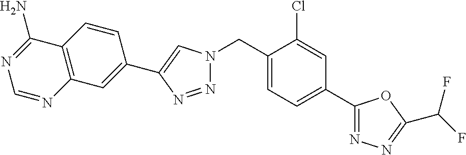 [SMILES: Nc1ncnc2cc(-c3cn(Cc4ccc(-c5nnc(C(F)F)o5)cc4Cl)nn3)ccc12] | 455.14 |

### Example 35. Synthesis of 4-(5-(1-(4-(5-(difluoromethyl)-1,3,4-oxadiazol-2-yl)benzyl)-1H-1,2,3-triazol-4-yl)-1H-benzo[d]imidazol-2-yl)morpholine (Compd. 174)

### Step A

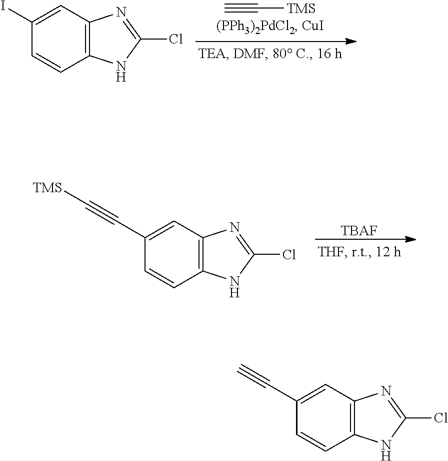

2-Chloro-5-iodo-1H-benzimidazole (500 mg, 1.8 mmol, 1 equiv.), ethynyl(trimethyl)silane (1.2 equiv.) and triethylamine (1.5 equiv.) were dissolved in 5 mL DMF and the resulting mixture was degassed. Bis(triphenylphosphine)palladium (II) chloride (0.1 equiv.) and copper(I) iodide (0.1 equiv.) were added and, after degassing, the reaction mixture was stirred at 80° C. overnight. Conversion of the starting material to the TMS-protected intermediate was monitored by LC-MS. After cooling the mixture to r.t., tetrabutylammonium fluoride (2 equiv., 1M THF solution) was added. The reaction mixture was stirred at r.t. over 12 h. The reaction mixture was quenched with sat. aq. NH₄C₁. The product was then extracted with EtOAc, washed with water (2×), and sat. aq. NaHCO₃ (2×). The organic extracts were combined and dried over Na₂SO₄, filtered, and concentrated. The residue was purified by flash column chromatography (DCM/MeOH 95:5) to give product (292 mg, 1.48 mmol, 82% yield).

Step B

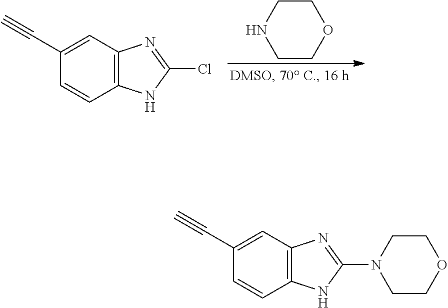 [SMILES: C#Cc1ccc2[nH]c(Cl)nc2c1 ; C1COCCN1 ; C#Cc1ccc2[nH]c(N3CCOCC3)nc2c1]

Morpholine (8 equiv.) was added to a solution of 2-chloro-5-ethynyl-1H-benzo[d]imidazole (40 mg, 0.23 mmol, 1 equiv.) in 1 mL DMSO. The reaction mixture was stirred at 70° C. overnight. 75% conversion was detected by LC-MS. Excess of morpholine was removed by evaporation. The residual DMSO solution was used in the next step without further purification.

Step C

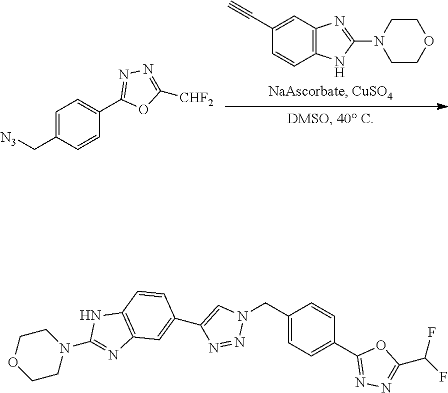 [SMILES: C#Cc1ccc2[nH]c(N3CCOCC3)nc2c1 ; [N-]=[N+]=NCc1ccc(-c2nnc(C(F)F)o2)cc1 ; FC(F)c1nnc(-c2ccc(Cn3cc(-c4ccc5[nH]c(N6CCOCC6)nc5c4)nn3)cc2)o1]

2-(4-(azidomethyl)phenyl)-5-(difluoromethyl)-1,3,4-oxadiazole (Intermediate G, 1 equiv.) was added to 4-(5-ethynyl-1H-benzo[d]imidazol-2-yl)morpholine DMSO solution obtained in step E (0.17 mmol, 1 equiv., 0.17M solution). Copper(II) sulfate pentahydrate (0.25 equiv., 0.1 M aqueous solution) and sodium L-ascorbate (0.5 equiv., 0.2 M aqueous solution) were added, and the reaction mixture was agitated at r.t. overnight. The reaction mixture was submitted to prep-HPLC (basic conditions) without any prior workup, to obtain pure target compound (17 mg, 0.033 mmol, 14% yield over two steps, m/z 479.5 [MH+]).

The following compound was prepared according to the same procedure:

| Compd. | Structure | m/z [MH+] |
|---|---|---|
| 190 | 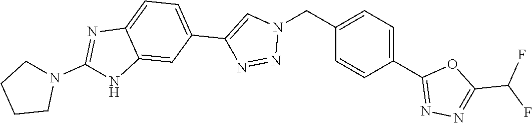 [SMILES: FC(F)c1nnc(-c2ccc(Cn3cc(-c4ccc5nc(N6CCCC6)[nH]c5c4)nn3)cc2)o1] | 463.14 |

The following compounds were synthesized according to the same procedure, excluding step B:

| Compd. | Structure | m/z [MH+] |
|---|---|---|
| 8 | 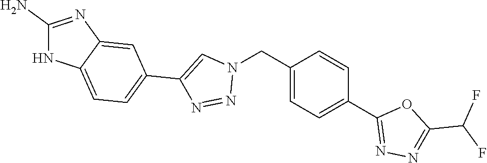 [SMILES: Nc1nc2cc(-c3cn(Cc4ccc(-c5nnc(C(F)F)o5)cc4)nn3)ccc2[nH]1] | 409.06 |
| 9 | 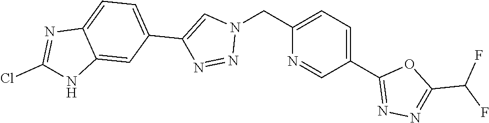 [SMILES: FC(F)c1nnc(-c2ccc(Cn3cc(-c4ccc5nc(Cl)[nH]c5c4)nn3)nc2)o1] | 429.2 |
| 30 | 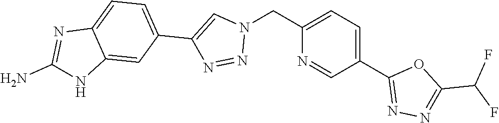 [SMILES: Nc1nc2ccc(-c3cn(Cc4ccc(-c5nnc(C(F)F)o5)cn4)nn3)cc2[nH]1] | 409.91 |
| 136 | 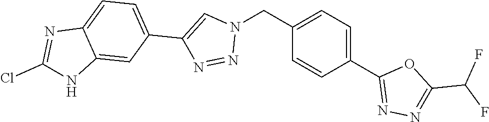 [SMILES: FC(F)c1nnc(-c2ccc(Cn3cc(-c4ccc5nc(Cl)[nH]c5c4)nn3)cc2)o1] | 428.4 |

### Example 36. Synthesis of 8-(1-(4-(5-(difluoromethyl)-1,3,4-oxadiazol-2-yl)-2,6-difluorobenzyl)-1H-1,2,3-triazol-4-yl)-4-methyl-1,3,4,5-tetrahydro-2H-benzo[e][1,4]diazepin-2-one (Compd. 237)

Step A

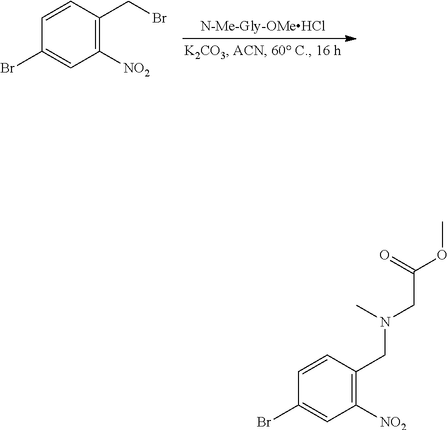 [SMILES: O=[N+]([O-])c1cc(Br)ccc1CBr ; COC(=O)CN(C)Cc1ccc(Br)cc1[N+](=O)[O-]]

A mixture of 4-bromo-1-(bromomethyl)-2-nitrobenzene (5.3 g, 17.9 mmol, 1 equiv.), sarcosine methyl ester (2.5 g, 17.9 mmol, 1 equiv.) and potassium carbonate (1.5 equiv.) in acetonitrile (50 mL) was heated to 60° C. and stirred overnight. The reaction mixture was then diluted with water and extracted with EtOAc. Combined organic layers were dried over Na₂SO₄, filtered and concentrated under reduced pressure. Crude product was purified on column chromatography (silica gel, 20% hexane/DCM) to obtain a yellow oil (2.37 g, 7.47 mmol, 42% yield).

Step B

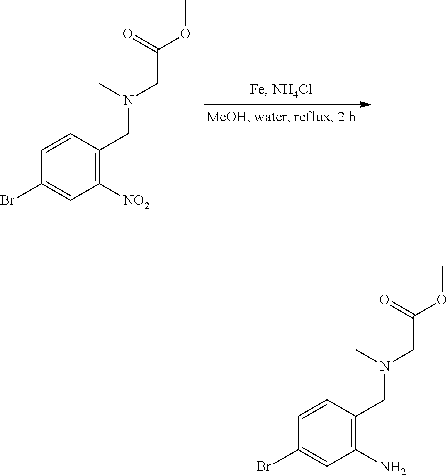 [SMILES: COC(=O)CN(C)Cc1ccc(Br)cc1[N+](=O)[O-] ; COC(=O)CN(C)Cc1ccc(Br)cc1N]

Methyl N-(4-bromo-2-nitrobenzyl)-N-methylglycinate (1.5 g, 4.7 mmol, 1 equiv.) was dissolved in MeOH (40 mL) and iron powder (5 equiv.) was added in small portions. The reaction mixture was heated to 70° C. and ammonium chloride (10 equiv., 4.7M aq. sol.) was added dropwise. The resulting mixture was then refluxed over 1 h. After addition of ammonium chloride, the mixture turned from yellow to brown and became turbid. After 1 h of heating, full conversion was observed by TLC. The reaction mixture was filtered on a Celite® pad, which was then washed with MeOH. The filtrate was concentrated, the resulting residue was dissolved in water and extracted with EtOAc. The combined organic phases were dried over Na₂SO₄, filtered and concentrated affording product as a brown oil (1.26 g, 4.26 mmol, 90% yield).

Step C

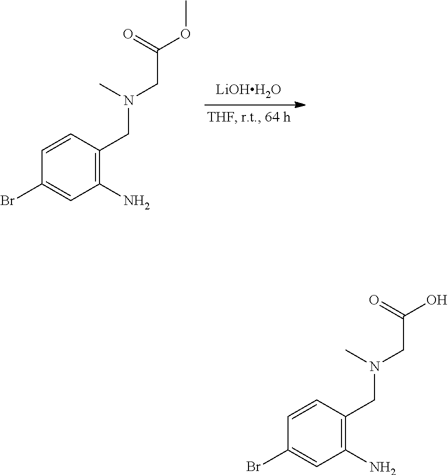 [SMILES: COC(=O)CN(C)Cc1ccc(Br)cc1N ; "LiOH·H2O" ; CN(CC(=O)O)Cc1ccc(Br)cc1N]

Methyl N-(2-amino-4-bromobenzyl)-N-methylglycinate (1.26 g, 4.26 mmol, 1 equiv.) was dissolved in 20 mL THF and lithium hydroxide monohydrate (3 equiv., 1.2M aq. sol) was added dropwise. The resulting mixture was stirred at r.t. over weekend. Reaction mixture was then diluted with water and pH was adjusted to 4 by careful addition of 4M HCl. Product was then extracted with EtOAc. Combined organic phases were dried over Na₂SO₄, filtered and concentrated (1.2 g, 4.12 mmol, 97% yield).

Step D

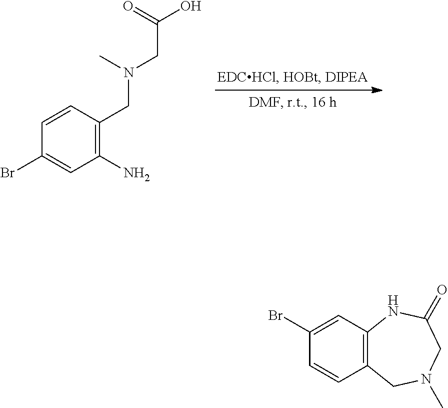

N-(2-amino-4-bromobenzyl)-N-methylglycine (654 mg, 2.22 mmol, 1 equiv.), 1-(3-Dimethylaminopropyl)-3-ethylcarbodiimide hydrochloride (1.6 equiv.) and HOBt (1.6 equiv.) were dissolved in 10 mL DMF. After stirring the mixture for 10 min, N,N-diisopropylethylamine (5 equiv.) was added. The resulting reaction mixture was stirred at r.t. overnight. The reaction mixture was diluted with water and aq. NaHCO₃, and extracted with MTBE and BuOH. Organic phases were combined, dried and concentrated. Crude product was purified by flash column chromatography (DCM/hexane 1:1, then DCM) (400 mg, 1.57 mmol, 70% yield).

Step E

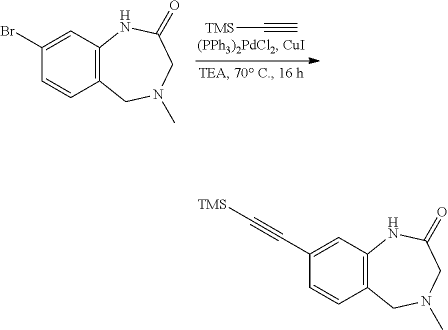

8-bromo-4-methyl-1,3,4,5-tetrahydro-2H-benzo[e][1,4]diazepin-2-one (184 mg, 0.69 mmol, 1 equiv.) was dissolved in triethylamine (3.9 mL, 40 equiv.) and the resulting mixture was degassed. Then ethynyl(trimethyl)silane (1.2 equiv.) was added to the reaction mixture, which was degassed. Bis(triphenylphosphine)palladium (II) chloride (0.02 equiv.) and copper(I) iodide (0.04 equiv.) were added and, after degassing, the reaction mixture was stirred at 70° C. overnight. The mixture was diluted with water and extracted with EtOAc. Combined organic phases were dried over MgSO₄, filtered and concentrated. Crude product was purified by flash chromatography (hexane/EtOAc to EtOAc) (107 mg, 0.356 mmol, 53% yield).

Step F

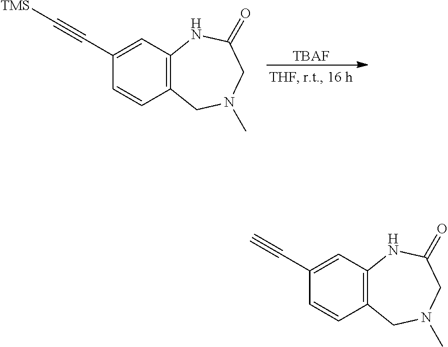

The 4-methyl-8-((trimethylsilypethynyl)-1,3,4,5-tetrahydro-2H-benzo[e][1,4]diazepin-2-one (107 mg, 0.356 mmol, 1 equiv.) was dissolved in 2 mL of THF and TBAF (2 equiv., 1M solution in THF) was added. The reaction mixture was stirred overnight, then diluted with EtOAc, washed with water and brine. The organic layer was dried over Na₂SO₄ and concentrated affording 131 mg of the light brown solid. Product purity was sufficient to proceed with the subsequent step (70 mg, 0.35 mmol, 96% yield). Step G

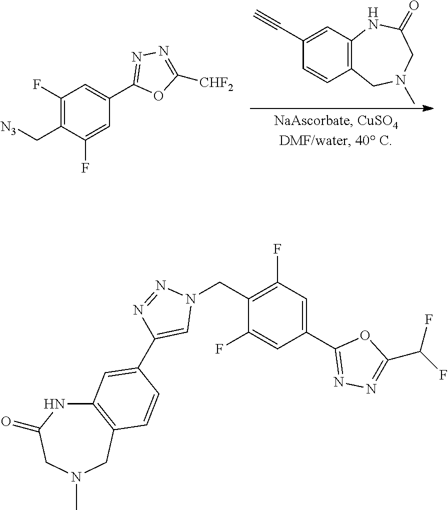 [SMILES: [N-]=[N+]=NCc1c(F)cc(-c2nnc(C(F)F)o2)cc1F ; C#Cc1ccc2c(c1)NC(=O)CN(C)C2 ; CN1CC(=O)Nc2cc(-c3cn(Cc4c(F)cc(-c5nnc(C(F)F)o5)cc4F)nn3)ccc2C1]

Copper(II) sulfate pentahydrate (0.1 equiv., 0.07 M aqueous solution) and sodium L-ascorbate (0.5 equiv., 0.35 M aqueous solution) were added to a solution of 2-(4-(azidomethyl)-3,5-difluorophenyl)-5-(difluoromethyl)-1,3,4-oxadiazole (Intermediate I, 10 mg, 0.035 mmol, 1 equiv.) and 8-ethynyl-4-methyl-1,3,4,5-tetrahydro-2H-benzo[e][1,4]diazepin-2-one (7 mg, 0.035 mmol, 1 equiv.) in 200 μL DMF. The reaction mixture was agitated at 35° C. overnight. Full conversion was detected by LC-MS. The reaction mixture was submitted to prep-HPLC (basic conditions) without any prior workup, to obtain pure target compound (3.5 mg, 0.007 mmol, 20% yield, m/z 488.11 [MH+]).

### Example 37. Synthesis of N-(3-(1-((5-(5-(difluoromethyl)-1,3,4-oxadiazol-2-yl)pyridin-2-yl)methyl)-1H-1,2,3-triazol-4-yl)phenyl)-4-methylpiperazine-1-carboxamide (Compd. 51)

Step A

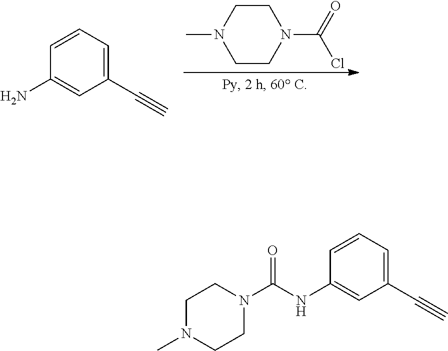

3-Ethynylaniline (100 mg, 0.85 mmol, 1 equiv.) was dissolved in 1 mL pyridine, and 4-methylpiperazine-1-carbonyl chloride (1.1 equiv.) was added. The reaction mixture was stirred for 2 h at 60° C. Pyridine was then removed by evaporation, and crude residue was used in the next step without any further purification.

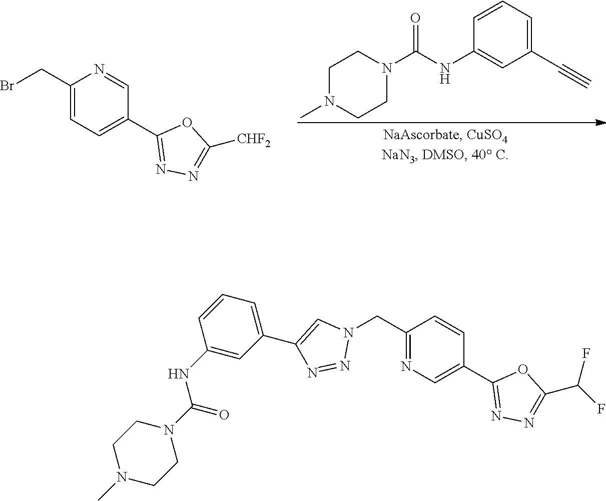 [SMILES: FC(F)c1nnc(-c2ccc(CBr)nc2)o1 ; C#Cc1cccc(NC(=O)N2CCN(C)CC2)c1 ; CN1CCN(C(=O)Nc2cccc(-c3cn(Cc4ccc(-c5nnc(C(F)F)o5)cn4)nn3)c2)CC1]

Crude N-(3-ethynylphenyl)-4-methylpiperazine-1-carboxamide obtained in the previous step (2.5 equiv.), 2-(6-(bromomethyl)pyridin-3-yl)-5-(difluoromethyl)-1,3,4-oxadiazole (Intermediate A, 0.34 mmol, 1 equiv.) and sodium azide (1 equiv.) were dissolved in 1 mL DMSO. Copper(II) sulfate pentahydrate (0.25 equiv., 0.2 M aqueous solution) and sodium L-ascorbate (0.5 equiv., 0.3 M aqueous solution) were added. The reaction mixture was stirred at r.t. overnight.

Crude mixture was submitted for prep-HPLC (ACN+0.1% FA, H₂O+0.1% FA) without any workup, obtaining 25 mg of the desired product (0.049 mmol, 14% yield, m/z 496.17 [MH+]).

The following compounds were synthesized according to the same procedure:

| Compd. | Structure | m/z [MH+] |
|---|---|---|
| 336 | 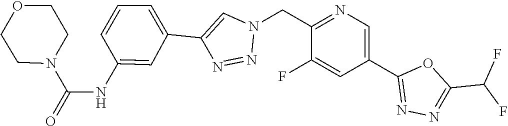 [SMILES: O=C(Nc1cccc(-c2cn(Cc3ncc(-c4nnc(C(F)F)o4)cc3F)nn2)c1)N1CCOCC1] | 501.21 |
| 337 | 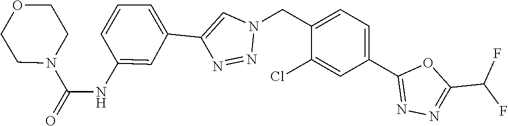 [SMILES: O=C(Nc1cccc(-c2cn(Cc3ccc(-c4nnc(C(F)F)o4)cc3Cl)nn2)c1)N1CCOCC1] | 516.19 |
| 338 | 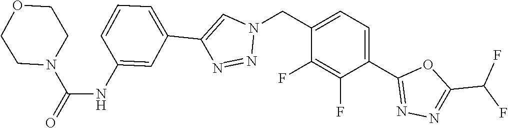 [SMILES: O=C(Nc1cccc(-c2cn(Cc3ccc(-c4nnc(C(F)F)o4)c(F)c3F)nn2)c1)N1CCOCC1] | 518.20 |
| 352 | 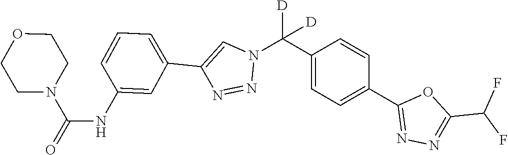 [SMILES: [2H]C([2H])(c1ccc(-c2nnc(C(F)F)o2)cc1)n1cc(-c2cccc(NC(=O)N3CCOCC3)c2)nn1] | 484.21 |
| 363 | 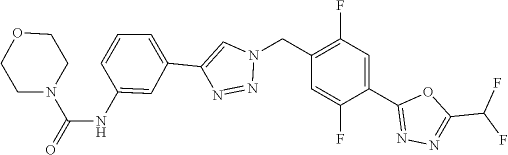 [SMILES: O=C(Nc1cccc(-c2cn(Cc3cc(F)c(-c4nnc(C(F)F)o4)cc3F)nn2)c1)N1CCOCC1] | 518.16 |
| 394 | 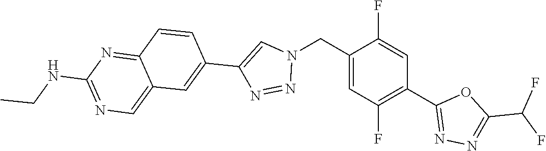 [SMILES: CCNc1ncc2cc(-c3cn(Cc4cc(F)c(-c5nnc(C(F)F)o5)cc4F)nn3)ccc2n1] | 485.2 |
| 395 | 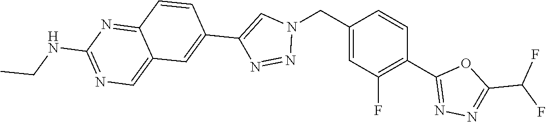 [SMILES: CCNc1ncc2cc(-c3cn(Cc4ccc(-c5nnc(C(F)F)o5)c(F)c4)nn3)ccc2n1] | 467.18 |

### Example 38. Synthesis of N-(3-(1-(4-(5-(difluoromethyl)-1,3,4-oxadiazol-2-yl)-2,6-difluorobenzyl)-1H-1,2,3-triazol-4-yl)phenyl)-1-methylazetidine-3-carboxamide (Compd. 86)

Step A

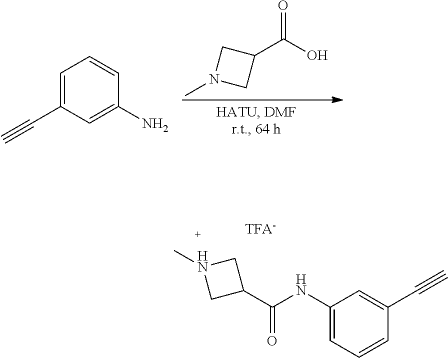

1-methylazetidine-3-carboxylic acid (1.3 equiv.) and HATU (1.3 equiv.) were suspended in 5 mL DMF and sonicated for 10 min until a clear solution was obtained. Then 3-ethynylaniline (763 mg, 6.5 mmol, 1 equiv.) was added, and the mixture was stirred at r.t. over 64 h. The reaction mixture was diluted with water and extracted with EtOAc. Organic phases were dried and evaporated. The crude product thus obtained was submitted for prep-HPLC (0.1% TFA/ACN/H₂O C-18). Evaporation of fractions gave 60 mg of the desired product (0.28 mmol, 3% yield), which was isolated as TFA salt.

Step B

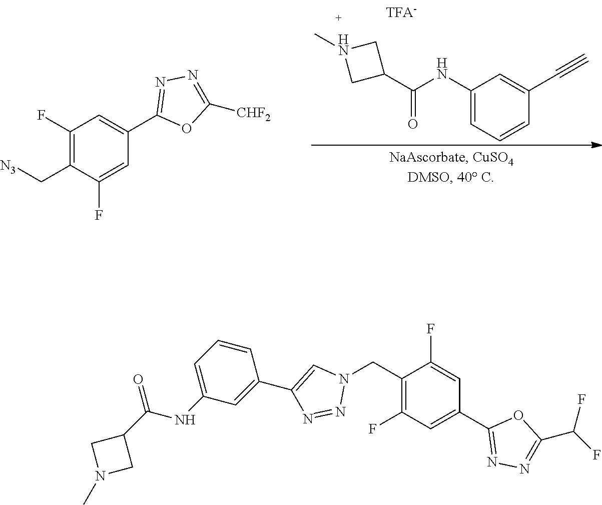

Copper(II) sulfate pentahydrate (0.1 equiv., 0.05 M aqueous solution) and sodium L-ascorbate (0.5 equiv., 0.25 M aqueous solution) were added to a solution of 2-(4-(azidomethyl)-3,5-difluorophenyl)-5-(difluoromethyl)-1,3,4-oxadiazole (Intermediate I, 24 mg, 0.084 mmol, 1.1 equiv.) and N-(3-ethynylphenyl)-1-methylazetidine-3-carboxamide trifluoroacetate (25 mg, 0.076 mmol, 1 equiv.) in 300 μL DMSO. The reaction mixture was agitated at 40° C. overnight. Full conversion of the starting material was detected by LC-MS. The reaction mixture was submitted to prep-HPLC (basic conditions) without any prior workup, to obtain pure target compound (9.7 mg, 0.019 mmol, 25% yield, m/z 502.15 [MH+]).

The following compound was prepared according to the same procedure:

| Compd. | Structure | m/z [MH+] |
|---|---|---|
| 149 | 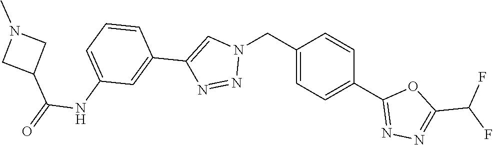 [SMILES: CN1CC(C(=O)Nc2cccc(-c3cn(Cc4ccc(-c5nnc(C(F)F)o5)cc4)nn3)c2)C1] | 466.2 |
| 336 | 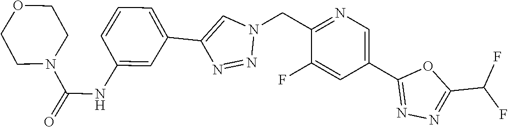 [SMILES: O=C(Nc1cccc(-c2cn(Cc3ncc(-c4nnc(C(F)F)o4)cc3F)nn2)c1)N1CCOCC1] | 501.21 |
| 337 | 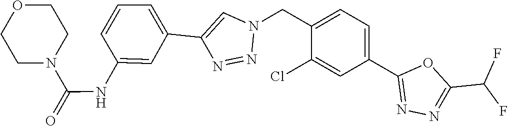 [SMILES: O=C(Nc1cccc(-c2cn(Cc3ccc(-c4nnc(C(F)F)o4)cc3Cl)nn2)c1)N1CCOCC1] | 516.19 |
| 338 | 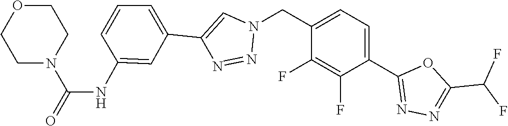 [SMILES: O=C(Nc1cccc(-c2cn(Cc3ccc(-c4nnc(C(F)F)o4)c(F)c3F)nn2)c1)N1CCOCC1] | 518.20 |
| 352 | 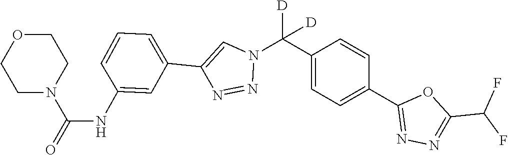 [SMILES: [2H]C([2H])(c1ccc(-c2nnc(C(F)F)o2)cc1)n1cc(-c2cccc(NC(=O)N3CCOCC3)c2)nn1] | 484.21 |
| 363 | 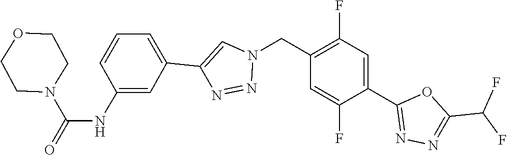 [SMILES: O=C(Nc1cccc(-c2cn(Cc3cc(F)c(-c4nnc(C(F)F)o4)cc3F)nn2)c1)N1CCOCC1] | 518.16 |
| 369 | 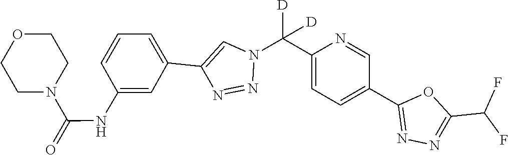 [SMILES: [2H]C([2H])(c1ccc(-c2nnc(C(F)F)o2)cn1)n1cc(-c2cccc(NC(=O)N3CCOCC3)c2)nn1] | 485.14 |

### Example 39. Synthesis of N-(5-(1-((5-(5-(difluoromethyl)-1,3,4-oxadiazol-2-yl)pyrimidin-2-yl)methyl)-1H-1,2,3-triazol-4-yl)pyridin-2-yl)-2,2-difluoroacetamide (Compd. 88)

Step A

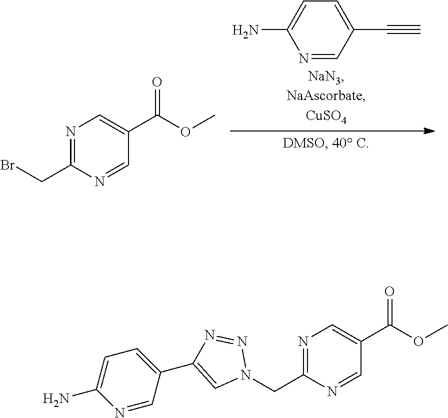

Copper(II) sulfate pentahydrate (0.3 equiv., 0.2 M aq. solution) and sodium L-ascorbate (0.5 equiv., 0.34 M aq. solution) were added to a stirring solution methyl 2-(bromomethyl)pyrimidine-5-carboxylate (63 mg, 0.34 mmol, 1 equiv.), 5-ethynylpyridin-2-amine (1 equiv.) and sodium azide (1.05 equiv.) in DMSO (1 mL). The resulting mixture was stirred at r.t. for 2 h. Full conversion was confirmed by LC-MS. The reaction mixture was diluted with EtOAc, washed with water, sat. aq. NaHCO₃ (3 times) and brine. Organic phase was dried over MgSO₄ and evaporated under reduced pressure. The residue obtained was used in the next step without purification (48 mg, 0.15 mmol, 46% yield).

Step B

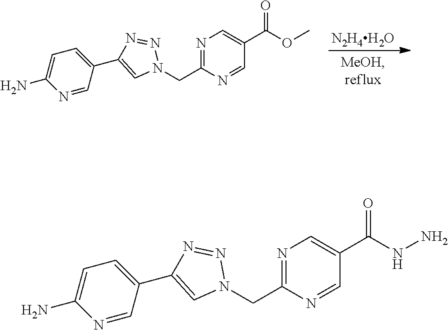

A suspension of methyl 2-((4-(6-aminopyridin-3-yl)-1H-1,2,3-triazol-1-yl)methyl)pyrimidine-5-carboxylate (45 mg, 0.145 mmol, 1 equiv.) and hydrazine monohydrate (5 equiv.) in MeOH (1 mL) was stirred at 70° C. over 3 h. Full conversion to the desired product was observed by LC-MS. The reaction mixture was evaporated in vacuum and reevaporated from acetonitrile twice to give target compound as an off-white suspension (45 mg, 0.145 mmol, 100% yield). The product was used for the subsequent step without further purification.

Step C

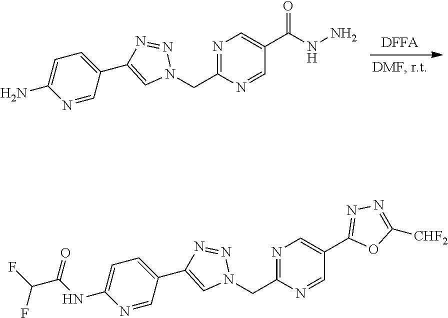 [SMILES: NNC(=O)c1cnc(Cn2cc(-c3ccc(N)nc3)nn2)nc1 ; "DFFA" ; O=C(Nc1ccc(-c2cn(Cc3ncc(-c4nnc(C(F)F)o4)cn3)nn2)cn1)C(F)F]

Difluoroacetic anhydride (6 equiv.) was added in portions to a solution of 2-((4-(6-aminopyridin-3-yl)-1H-1,2,3-triazol-1-yl)methyl)pyrimidine-5-carbohydrazide (35 mg, 0.085 mmol, 1 equiv.) in DMF (2 mL). After 2 h the reaction was complete, the main product being the target compound. The reaction mixture was diluted with EtOAc, washed with sat. aq. NaHCO₃ (4 times) and brine, dried over MgSO₄ and evaporated under vacuum. The crude residue was submitted for prep-HPLC purification. After purification, 18 mg of pure compound were obtained (0.039 mmol, 27% yield, m/z 449.89 [MH+]).

### Example 40. Synthesis of 5-(1-(1-(4-(5-(difluoromethyl)-1,3,4-oxadiazol-2-yl)phenyl)ethyl)-1H-1,2,3-triazol-4-yl)pyridin-2-amine (Compd. 117)

Step A

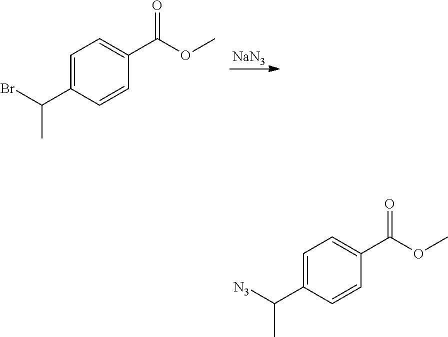

A solution of methyl 4-(1-bromoethyl)benzoate (2 g, 8.22 mmol, 1 equiv.) in DMSO (10 mL) was added to a solution of sodium azide (1.4 equiv.) in DMSO. The reaction mixture was vigorously stirred at r.t. overnight. The reaction was quenched with water (200 mL) and the product extracted with EtOAc (3 times). The organic layers were collected together, washed with brine, dried over MgSO₄, and concentrated under reduced pressure to afford the product as a colorless oil which was used in the next step without any further purification (1.69 g, 8.22 mmol, 100% yield).

Step B

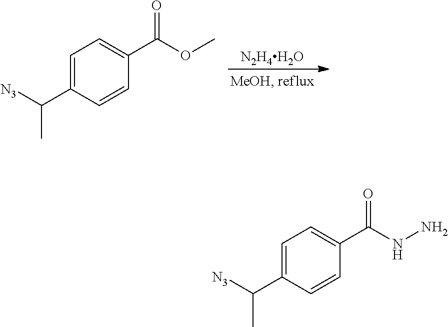

Methyl 4-(1-azidoethyl)benzoate (1.69 g, 8.22 mmol, 1 equiv.) was dissolved in MeOH (20 mL), then hydrazine monohydrate was added (5 equiv.) under stirring. Mixture was stirred at 70° C. overnight. Full conversion of methyl ester to hydrazide was observed by LC-MS (and TLC). The reaction mixture was concentrated under reduced pressure and the residue was diluted in water and extracted with ethyl acetate. The organic phase was washed with sat. aq. NaHCO₃, brine, dried, filtered and concentrated under reduced pressure. The product obtained (1.69 g, 8.22 mmol, 100% yield) was used for the subsequent step without any further purification.

Step C

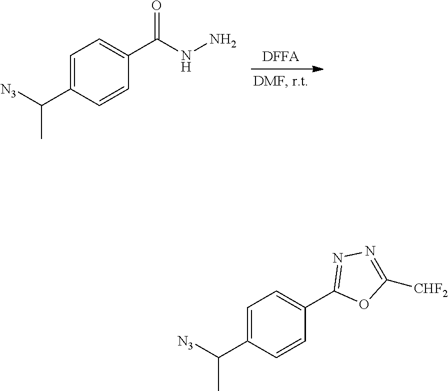 [SMILES: CC(N=[N+]=[N-])c1ccc(C(=O)NN)cc1 ; "DFFA" ; CC(N=[N+]=[N-])c1ccc(-c2nnc(C(F)F)o2)cc1]

4-(1-azidoethyl)benzohydrazide (844 mg, 4.1 mmol, 1 equiv.) was dissolved in dry DMF (10 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. Full conversion was observed by LC-MS. Aqueous NaHCO₃ was added to the reaction mixture to quench difluoroacetic anhydride excess. Then water was added, and the product was extracted with ethyl acetate (3×). Organic layers were collected together, washed with sat. aq. NaHCO₃ and brine, dried over Na₂SO₄ and evaporated to dryness under reduced pressure. The crude residue was purified by flash column chromatography (hexane/EtOAc 95:5) affording the product as a yellow oil (506 mg, 1.9 mmol, 46% yield).

Step D

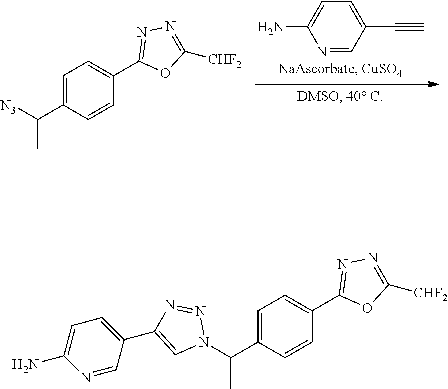

Copper(II) sulfate pentahydrate (0.3 equiv., 0.5 M aqueous solution) and sodium L-ascorbate (0.5 equiv., 1M aq. sol.) were added to a solution of 2-(4-(1-azidoethyl)phenyl)-5-(difluoromethyl)-1,3,4-oxadiazole (78 mg, 0.296 mmol, 1 equiv.) and 5-ethynylpyridin-2-amine (35 mg, 0.296 mmol, 1 equiv.) in 1 mL DMSO. The reaction mixture was agitated at 40° C. over 2 h. Full conversion of the starting material was detected by LC-MS. Reaction mixture was filtered through syringe filter and submitted to prep-HPLC without any further work up. After evaporation of fractions 67 mg of target compound (0.169 mmol, 57% yield) were obtained as an off-white solid (m/z 384.14 [MH+]).

### Example 41. Synthesis of 2-(4-(6-aminopyridin-3-yl)-1H-1,2,3-triazol-1-yl)-2-(4-(5-(difluoromethyl)-1,3,4-oxadiazol-2-yl)phenyl)ethan-1-ol (Compd. 94)

Step A

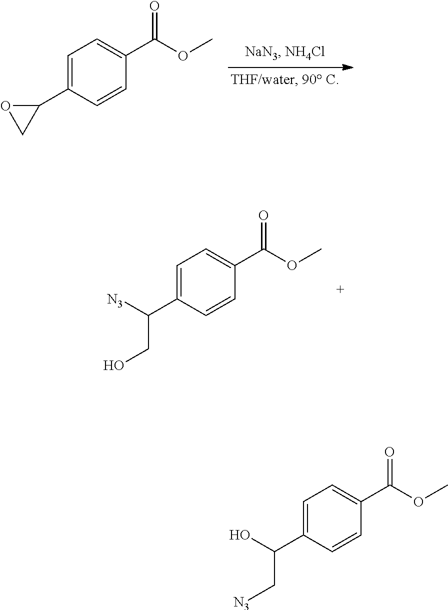 [SMILES: COC(=O)c1ccc(C2CO2)cc1 ; COC(=O)c1ccc(C(CO)N=[N+]=[N-])cc1 ; COC(=O)c1ccc(C(O)CN=[N+]=[N-])cc1]

Sodium azide (2 equiv.) and ammonium chloride (2 equiv.) were dissolved in 2 mL water. Methyl 4-(oxiran-2-yl)benzoate (600 mg, 3.36 mmol, 1 equiv.) was added as a solution in 8 mL THF. The reaction mixture was stirred at 90° C. overnight. Almost full conversion was observed by LC-MS. The reaction mixture was diluted with EtOAc and washed with water (3 times) and brine. Organic phase was dried over Na₂SO₄, filtered, concentrated. The crude product thus obtained was an inseparable mixture of regioisomers, which was used in the next step without further purification (600 mg, 2.7 mmol, 81% yield).

Step B

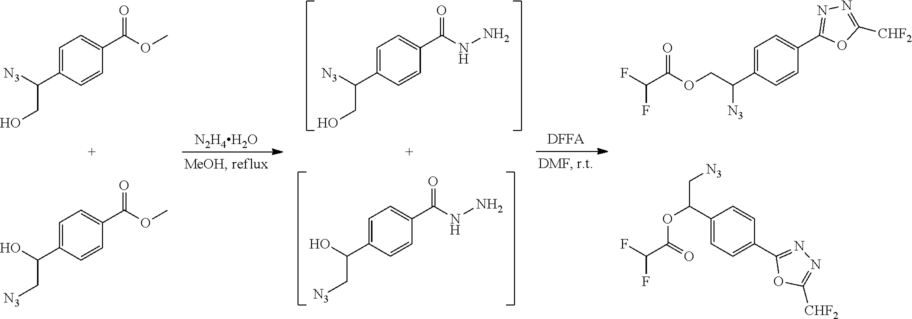

The regioisomeric mixture obtained in step A (600 mg, 2.7 mmol, 1 equiv.) was dissolved in MeOH (10 mL), then hydrazine monohydrate was added (5 equiv.) under stirring. Mixture was stirred at 70° C. overnight. Full conversion of methyl esters to the corresponding hydrazides was observed by LC-MS. The reaction mixture was concentrated under reduced pressure and the residue was diluted in water and extracted with ethyl acetate. The organic phase was washed with sat. aq. NaHCO₃ and brine, dried over MgSO₄, filtered and concentrated under reduced pressure. The residue was resuspended in dry DMF (10 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. Full conversion was observed by LC-MS.

Sat. aq. NaHCO₃ was added to the reaction mixture to quench the excess of difluoroacetic anhydride. Then water was added, and the product was extracted with ethyl acetate (3×). Organic layers were collected together, washed with sat. aq. NaHCO₃ and brine, dried over Na₂SO₄ and evaporated to dryness under reduced pressure. The crude residue was purified by flash chromatography (hexane/EtOAc 8:2) affording the product as a mixture of isomers (176 mg, 0.5 mmol, 18% yield).

Step C

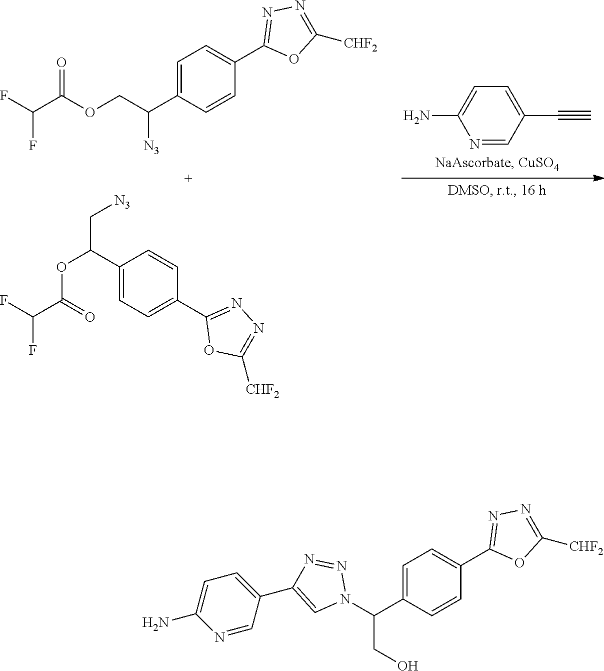

Copper(II) sulfate pentahydrate (0.1 equiv., 0.5 M aqueous solution) and sodium L-ascorbate (0.5 equiv., 1 M aqueous solution) were added to a solution of the azides obtained in step B (176 mg, 0.5 mmol, 1 equiv.) and 5-ethynylpyridin-2-amine (58 mg, 0.5 mmol, 1 equiv.) in 10 mL DMSO. The reaction mixture was agitated at r.t. overnight. The reaction mixture was submitted to prep-HPLC without any further work up. The mixture of isomers thus obtained was further purified by phenyl column to isolate the desired product as formate (6.4 mg, 0.014 mmol, 3% yield). Structure was proven by NOESY. (m/z 400.36 [MH+]).

The following compounds were synthesized according to the same procedure:

| Compd. | Structure | m/z [MH+] |
|---|---|---|
| 302 | 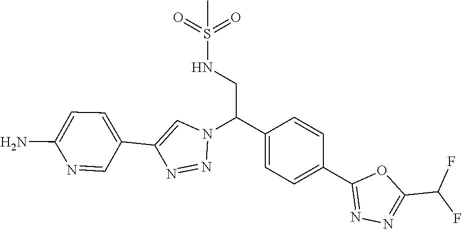 [SMILES: CS(=O)(=O)NCC(c1ccc(-c2nnc(C(F)F)o2)cc1)n1cc(-c2ccc(N)nc2)nn1] | 477.05 |
| 314 | 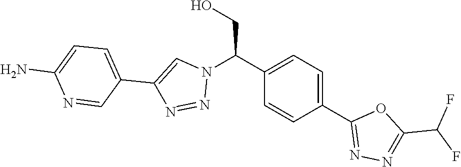 [SMILES: Nc1ccc(-c2cn([C@@H](CO)c3ccc(-c4nnc(C(F)F)o4)cc3)nn2)cn1] | 400.02 |

### Example 42. Synthesis of 5-(1-(1-(4-(5-(difluoromethyl)-1,3,4-oxadiazol-2-yl)phenyl)ethyl)-1H-1,2,3-triazol-4-yl)pyridin-2-amine (Compd. 158)

Step A

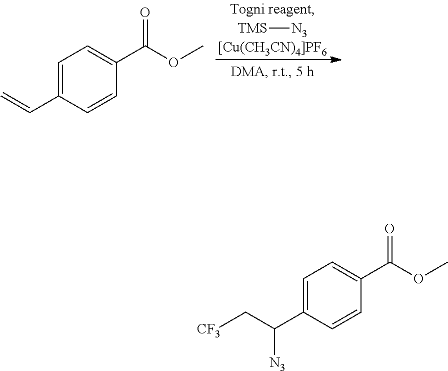

Togni's reagent (1.5 equiv.) and tetrakis(acetonitrile)copper(I) hexafluorophosphate (0.05 equiv.) were dissolved in 5 mL DMA in a dried sealed tube purged with argon. Methyl 4-vinylbenzoate (160 mg, 0.99 mmol, 1 equiv.) and trimethylsilyl azide (2 equiv.) were added. The reaction mixture was stirred at r.t. for 5 h. The mixture was diluted with ethyl acetate, and sequentially washed with water and brine. The organic layer was concentrated under vacuum. The yellow oil residue was purified by flash chromatography (hexane/AcOEt 96:4 to 3:1) to afford the product as a colorless oil (130 mg, 0.48 mmol, 48% yield).

Step B

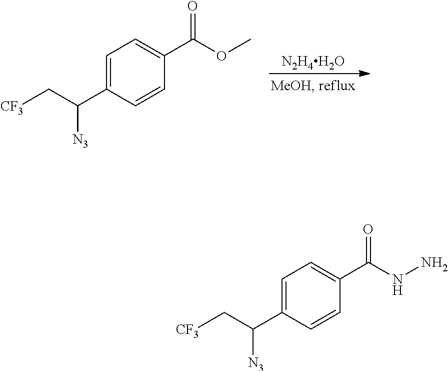

Methyl 4-(1-azido-3,3,3-trifluoropropyl)benzoate (130 mg, 0.48 mmol, 1 equiv.) was dissolved in MeOH (2 mL), then hydrazine monohydrate was added (5 equiv.) under stirring. Mixture was stirred at 70° C. overnight. Full conversion of methyl ester to hydrazide was observed by LC-MS (and TLC). The reaction mixture was concentrated under reduced pressure and the residue was diluted in water and extracted with ethyl acetate. The organic phase was washed with sat. aq. NaHCO₃, brine, dried, filtered and concentrated under reduced pressure. The product (130 mg, 0.404 mmol, 100% yield) was used for the subsequent step without any further purification.

Step C

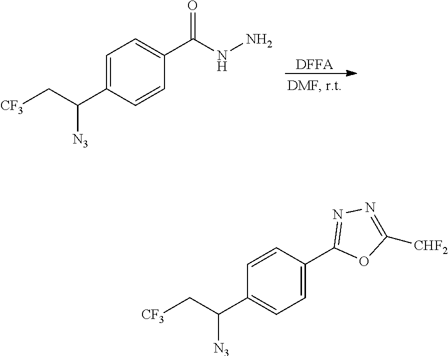

4-(1-azido-3,3,3-trifluoropropyl)benzohydrazide (130 mg, 0.404 mmol, 1 equiv.) was dissolved in dry DMF (1.5 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. 85% conversion was observed by LC-MS. Water was added to the reaction mixture which was extracted with ethyl acetate (3×). Organic layers were collected together, washed with sat. aq. NaHCO₃ and brine, dried over MgSO₄ and evaporated to dryness under reduced pressure. The crude residue was purified by flash column chromatography (hexane/EtOAc 9:1 to 8:2) affording the product as a colorless oil (73 mg, 0.217 mmol, 46% yield).

Step D

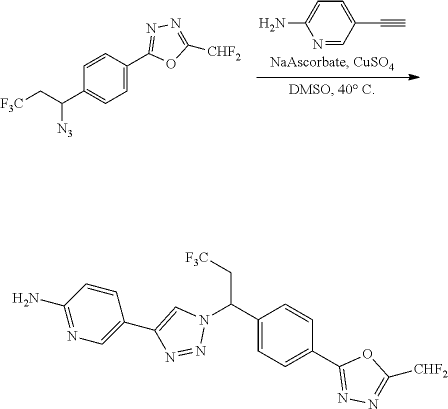

Copper(II) sulfate pentahydrate (0.2 equiv., 0.5 M aqueous solution) and sodium L-ascorbate (0.4 equiv., 1 M aqueous solution) were added to a solution of 2-(4-(1-azido-3,3,3-trifluoropropyl)phenyl)-5-(difluoromethyl)-1,3,4-oxadiazole (70 mg, 0.21 mmol, 1 equiv.) and 5-ethynylpyridin-2-amine (25 mg, 0.21 mmol, 1 equiv.) in 1.2 mL DMSO. The reaction mixture was stirred at r.t. overnight. Full conversion of the starting material was detected by LC-MS. The reaction mixture was diluted with water and extracted with ethyl acetate. The organic layer was washed with sat. aq. NaHCO₃ and brine, dried over MgSO₄, filtered and concentrated under reduced pressure. The crude residue was purified by prep-HPLC using neutral conditions affording the product as a white solid (44 mg, 0.097 mmol, 46% yield, m/z 452.12 [MH+]).

### Example 43. Synthesis of 5-(1-(1-(4-(5-(difluoromethyl)-1,3,4-oxadiazol-2-yl)phenyl)-2-(pyrrolidin-1-yl)ethyl)-1H-1,2,3-triazol-4-yl)pyridin-2-amine (Compd. 124)

Step A

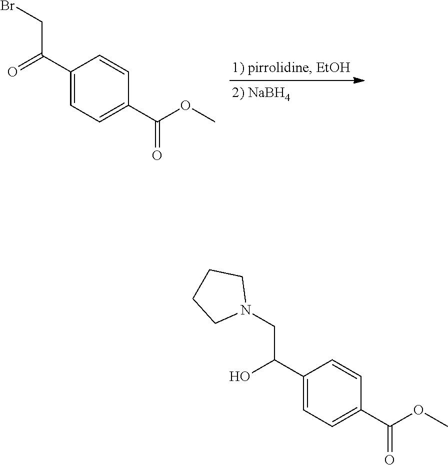 [SMILES: COC(=O)c1ccc(C(=O)CBr)cc1 ; COC(=O)c1ccc(C(O)CN2CCCC2)cc1]

Methyl 4-(2-bromoacetyl)benzoate (600 mg, 2.3 mmol) was dissolved in 7 mL ethanol and pyrrolidine (2 equiv.) was added. The reaction mixture was stirred at r.t. overnight. 90% conversion to intermediate ketone was detected by LC-MS. Sodium borohydride (1.1 equiv.) was added in portions to the reaction mixture, which was stirred at r.t. for 1 h. Full reduction to the corresponding alcohol intermediate was detected. The reaction mixture was diluted with EtOAc and washed with brine (3 Times). Organic layer was dried over Na₂SO₄ and concentrated under reduced pressure.

Step B

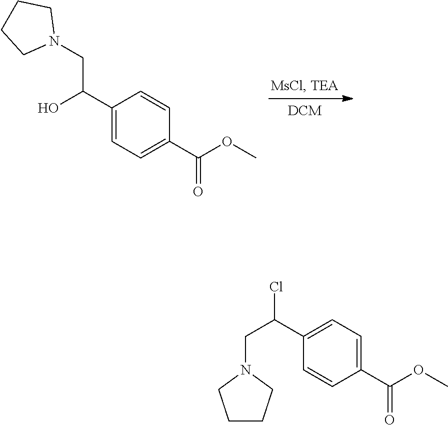 [SMILES: COC(=O)c1ccc(C(O)CN2CCCC2)cc1 ; COC(=O)c1ccc(C(Cl)CN2CCCC2)cc1]

Crude methyl 4-(1-hydroxy-2-(pyrrolidin-1-yl)ethyl)benzoate (1 equiv.) obtained from step A was dissolved in 20 mL DCM, and triethylamine (2 equiv.) and mesyl chloride (1 equiv.) were added under stirring. The reaction mixture was stirred at r.t. overnight. According to LC-MS chlorination mainly occurred. The reaction mixture was diluted with EtOAc and washed with brine. Organic fraction was dried over Na₂SO₄, filtered and evaporated.

Step C

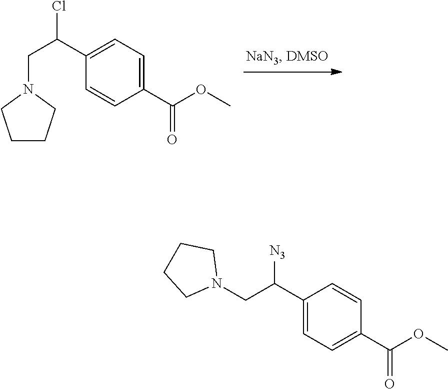 [SMILES: COC(=O)c1ccc(C(Cl)CN2CCCC2)cc1 ; COC(=O)c1ccc(C(CN2CCCC2)N=[N+]=[N-])cc1]

Crude residue obtained from step B was dissolved in DMSO, and sodium azide (1.2 equiv.) was added. The reaction mixture was stirred at r.t. for 1 h. Full conversion was observed by LC-MS. The product thus obtained (120 mg, 0.43 mmol, 19% yield over 3 steps) was used in the subsequent step without further purification.

Step D

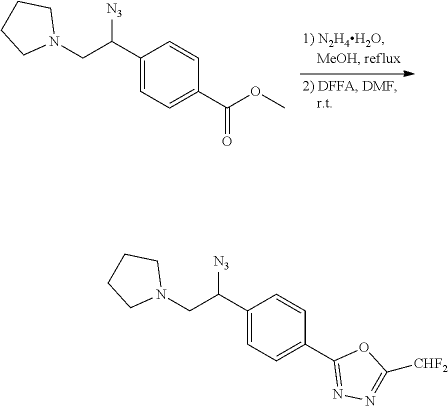 [SMILES: COC(=O)c1ccc(C(CN2CCCC2)N=[N+]=[N-])cc1 ; [N-]=[N+]=NC(CN1CCCC1)c1ccc(-c2nnc(C(F)F)o2)cc1]

Methyl 4-(1-azido-2-pyrrolidin-1-ylethyl)benzoate (120 mg, 0.43 mmol, 1 equiv.) was dissolved in MeOH (5 mL), then hydrazine monohydrate was added (5 equiv.) under stirring. The mixture was stirred at 70° C. overnight. Full conversion of methyl ester to hydrazide was observed by LC-MS (and TLC). The reaction mixture was concentrated under reduced pressure and the residue was diluted in water and extracted with ethyl acetate. The organic phase was washed with sat. aq. NaHCO₃, brine, dried, filtered and concentrated under reduced pressure. The crude residue was dissolved in dry DMF (3 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. Full conversion was observed by LC-MS. Sat. aq. NaHCO₃ was added to the reaction mixture to quench difluoroacetic anhydride excess. Then water was added, and the product was extracted with ethyl acetate (3 times). Organic layers were collected together, washed with sat. aq. NaHCO₃ and brine, dried over Na₂SO₄ and evaporated to dryness under reduced pressure. The crude residue was purified by flash column chromatography affording the product as a yellow semi-solid (100 mg, 0.3 mmol, 72% yield).

Step E

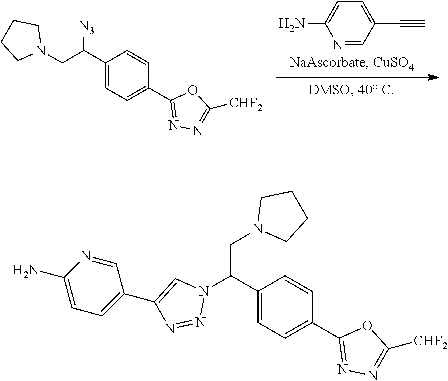

Copper(II) sulfate pentahydrate (0.15 equiv., 0.5 M aqueous solution) and sodium L-ascorbate (0.3 equiv., 1 M aqueous solution) were added to a solution of 2-(4-(1-azido-2-(pyrrolidin-1-yl)ethyl)phenyl)-5-(difluoromethyl)-1,3,4-oxadiazole (50 mg, 0.15 mmol, 1 equiv.) and 5-ethynylpyridin-2-amine (18 mg, 0.15 mmol, 1 equiv.) in 1 mL DMSO. The reaction mixture was stirred at 40° C. over 2 h. Full conversion of the starting material was detected by LC-MS. Reaction mixture was filtered through syringe filter and submitted to prep-HPLC without any further work up. After evaporation of fractions 26 mg of target compound (0.057 mmol, 38% yield) were obtained as a yellow solid (m/z 453.20 [MH+]).

The following compound was synthesized according to the same synthetic route:

| Compd. | Structure | m/z [MH+] |
|---|---|---|
| 236 | 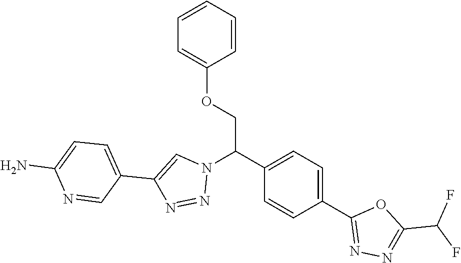 [SMILES: Nc1ccc(-c2cn(C(COc3ccccc3)c3ccc(-c4nnc(C(F)F)o4)cc3)nn2)cn1] | 476.6 |
| 312 | 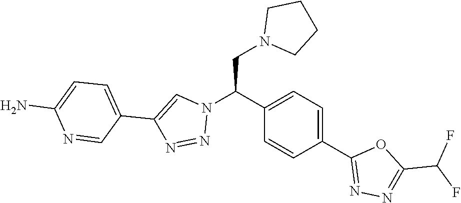 [SMILES: Nc1ccc(-c2cn([C@@H](CN3CCCC3)c3ccc(-c4nnc(C(F)F)o4)cc3)nn2)cn1] | 453.08 |
| 313 | 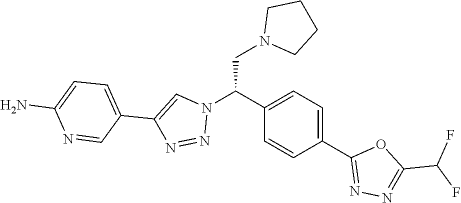 [SMILES: Nc1ccc(-c2cn([C@H](CN3CCCC3)c3ccc(-c4nnc(C(F)F)o4)cc3)nn2)cn1] | 453.09 |

### Example 44. Synthesis of 5-(1-(2-(4-chlorophenyl)-1-(4-(5-(difluoromethyl)-1,3,4-oxadiazol-2-yl)phenyl)ethyl)-1H-1,2,3-triazol-4-yl)pyridin-2-amine (Compd. 231)

Step A

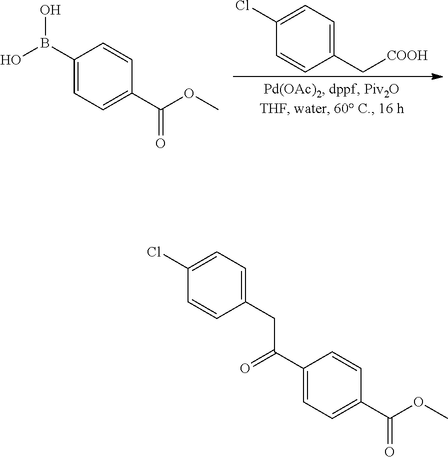 [SMILES: COC(=O)c1ccc(B(O)O)cc1 ; O=C(O)Cc1ccc(Cl)cc1 ; COC(=O)c1ccc(C(=O)Cc2ccc(Cl)cc2)cc1]

A reaction vessel equipped with a pressure equalizer was charged with palladium(II) acetate (0.030 equiv.), 1,1′-bis(diphenylphosphino)ferrocene (0.035 equiv.), 3-(4-chlorophenyl)propionic acid (500 mg, 2.93 mmol, 1 equiv.), and (4-(methoxycarbonyl)phenyl)boronic acid (1.2 equiv.). THF (4 mL), water (0.25 equiv.), and pivalic anhydride (1.5 equiv.) were successively added. The flask was purged with argon and the reaction mixture was heated at 60° C. overnight. After removal of the volatiles under reduced pressure, the residue was dissolved in a minimum amount of DCM, transferred on the top of a basic alumina pad, and eluted with hexane/EtOAc gradient. The crude product was further purified by flash column chromatography (hexane/AcOEt 4:1) (205 mg, 0.712 mmol, 25% yield).

Step B

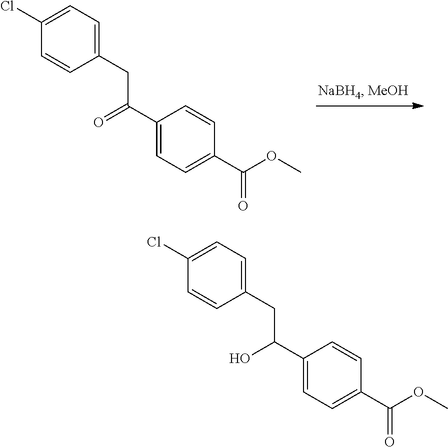

Methyl 4-[2-(4-chlorophenyl)acetyl]benzoate (205 mg, 0.712 mmol, 1 equiv.) was dissolved in 3 mL methanol. Sodium borohydride (1.5 equiv.) was added in portions to the reaction mixture at 0° C. The reaction mixture was stirred over 3 h. Full reduction to the corresponding alcohol intermediate was detected. The mixture was concentrated in vacuo. The residue was suspended in cold water to quench the excess of sodium borohydride. The mixture was extracted with DCM, and organic layer was dried over anhydrous Na₂SO₄ and concentrated by rotary evaporation. The product thus obtained (174 mg, 0.6 mmol, 82% yield) was used in the next step without further purification.

Step C

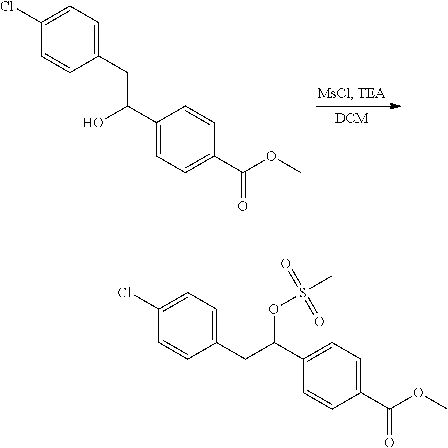 [SMILES: COC(=O)c1ccc(C(O)Cc2ccc(Cl)cc2)cc1 ; COC(=O)c1ccc(C(Cc2ccc(Cl)cc2)OS(C)(=O)=O)cc1]

Triethylamine (2 equiv.) and mesyl chloride (1.2 equiv.) were added to a solution of methyl 4-[2-(4-chlorophenyl)-1-hydroxyethyl]benzoate (174 mg, 0.6 mmol, 1 equiv.) in 10 mL dichloromethane at 0° C. The reaction mixture was let to reach r.t., and then stirred over 12 h. The mixture was then diluted with DCM, washed with water and brine, dried over Na₂SO₄. Volatiles were removed under reduced pressure, and the crude product thus obtained (215 mg, 0.58 mmol, 97% yield) was used in the subsequent step without further purification.

Step D

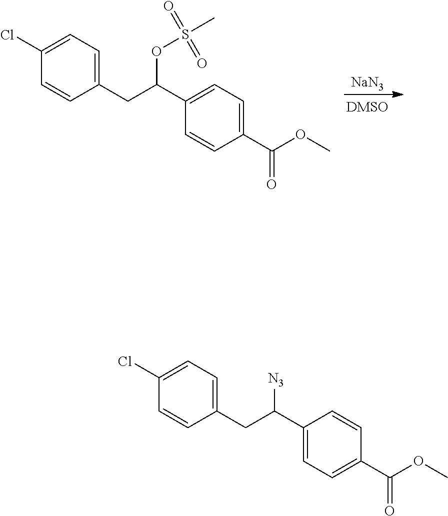 [SMILES: COC(=O)c1ccc(C(Cc2ccc(Cl)cc2)OS(C)(=O)=O)cc1 ; COC(=O)c1ccc(C(Cc2ccc(Cl)cc2)N=[N+]=[N-])cc1 ; "NaN3"]

Crude methyl 4-(2-(4-chlorophenyl)-1-((methylsulfonyl)oxy)ethyl)benzoate (215 mg, 0.58 mmol, 1 equiv.) was dissolved in 5 mL DMSO, and sodium azide (1.4 equiv.) was added. The reaction mixture was stirred at r.t. for 1 h. Full conversion was observed by LC-MS. The reaction was quenched with water and extracted with ethyl acetate. The organic layer was washed with brine, dried over Na₂SO₄, filtered and concentrated under reduced pressure. The residue was suspended in water and freeze-dried, affording a colorless oil (182 mg, 0.58 mmol, 99% yield) which was used in the next step without further purification.

Step E

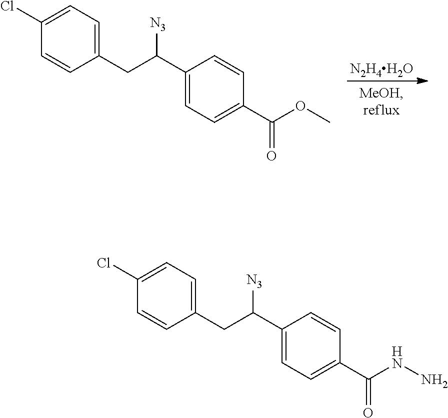 [SMILES: COC(=O)c1ccc(C(Cc2ccc(Cl)cc2)N=[N+]=[N-])cc1 ; "N2H4·H2O" ; [N-]=[N+]=NC(Cc1ccc(Cl)cc1)c1ccc(C(=O)NN)cc1]

A solution of methyl 4-(1-azido-2-(4-chlorophenyl)ethyl)benzoate (182 mg, 0.58 mmol, 1 equiv.) in methanol (5 mL) was added to hydrazine monohydrate (4 equiv.) under gentle stirring, dropwise. The mixture was refluxed overnight. Full conversion of methyl ester to hydrazide was observed by LC-MS (and TLC). The reaction mixture was concentrated under reduced pressure and the crude product thus obtained (171 mg, 0.54 mmol, 93% yield) was used for the next step without further purification.

Step F

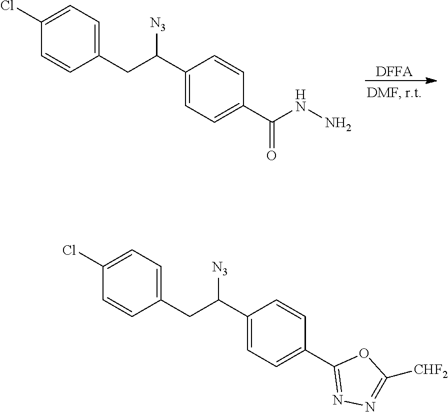 [SMILES: [N-]=[N+]=NC(Cc1ccc(Cl)cc1)c1ccc(C(=O)NN)cc1 ; "DFFA" ; [N-]=[N+]=NC(Cc1ccc(Cl)cc1)c1ccc(-c2nnc(C(F)F)o2)cc1]

4-(1-azido-2-(4-chlorophenyl)ethyl)benzohydrazide (171 mg, 0.54 mmol, 1 equiv.) was dissolved in dry DMF (5 mL) under argon. Difluoroacetic anhydride (3 equiv.) was slowly added, keeping temperature below 30° C. (ice/NaCl bath). After completing the addition, the temperature was let to reach r.t. The flask was sealed and the reaction mixture was stirred at r.t. overnight. Full conversion was observed by LC-MS. Sat. aq. NaHCO₃ was added to the reaction mixture to quench difluoroacetic anhydride excess. Then water was added, and the product was extracted with ethyl acetate (3×). Organic layers were collected together, washed with sat. aq. NaHCO₃ and brine, dried over Na₂SO₄ and evaporated to dryness under reduced pressure. The crude residue was purified by flash column chromatography (hexane/EtOAc 85:15) affording the product as a colorless oil (102 mg, 0.27 mmol, 50% yield).

Step G

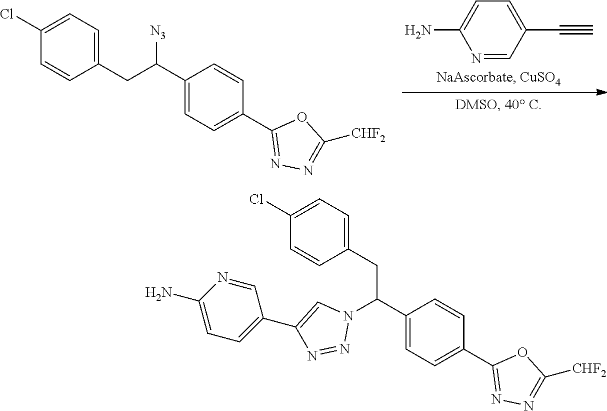

Copper(II) sulfate pentahydrate (0.1 equiv., 0.5M aq. sol.) and sodium L-ascorbate (0.5 equiv., 1M aq. sol.) were added to a solution of 2-(4-(1-azido-2-(4-chlorophenyl)ethyl)phenyl)-5-(difluoromethyl)-1,3,4-oxadiazole (50 mg, 0.13 mmol, 1 equiv.) and 5-ethynylpyridin-2-amine (15 mg, 0.13 mmol, 1 equiv.) in 1 mL DMSO. The reaction mixture was stirred at 40° C. over 2 h. Full conversion of the starting material was detected by LC-MS. The reaction mixture was filtered through syringe filter and submitted to prep-HPLC without any further work up. After evaporation of fractions 44 mg of target compound (0.089 mmol, 67% yield) were obtained as a beige solid (m/z 494.13 [MH+]).

### Example 45. Synthesis of 5-(1-(2-cyclobutyl-1-(4-(5-(difluoromethyl)-1,3,4-oxadiazol-2-yl)phenyl)ethyl)-1H-1,2,3-triazol-4-yl)pyridin-2-amine (Compd. 242)

Step A

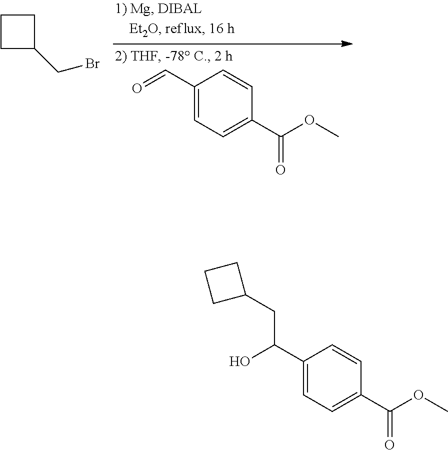 [SMILES: BrCC1CCC1 ; COC(=O)c1ccc(C=O)cc1 ; COC(=O)c1ccc(C(O)CC2CCC2)cc1]

A solution of DIBAL-H in hexane (1M, 0.02 equiv.) was added to a suspension of magnesium turnings (244 mg, 1.5 equiv., dried under vacuum) in anhydrous diethyl ether (4 mL) to initiate the reaction. Then, a few drops of a solution of (bromomethyl)cyclobutene (1 g, 6.7 mmol, 1 equiv.) in dry diethyl ether (4 mL) were added at r.t. After a few minutes, the rest of the solution was added. The resulting mixture was heated with a warm water bath and stirred overnight. This mixture was added dropwise to a solution of methyl 4-formylbenzoate (1.1 g, 6.7 mmol, 1 equiv.) in THF at −78° C. The reaction mixture was stirred for 2 h at −78° C. and at r.t. for additional 2 h. The reaction was quenched with water and extracted with ethyl acetate. The organic layers were combined and washed with brine, dried over MgSO₄, filtered and concentrated under reduced pressure. The residue was purified by flash column chromatography (hexane/EtOAc 9:1 to 7:3), affording the product as a yellow oil (375 mg, 1.6 mmol, 24% yield).

Step B

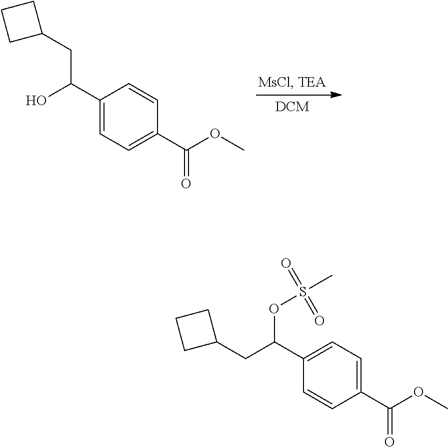 [SMILES: COC(=O)c1ccc(C(O)CC2CCC2)cc1 ; COC(=O)c1ccc(C(CC2CCC2)OS(C)(=O)=O)cc1]

Triethylamine (2 equiv.) and mesyl chloride (1.2 equiv.) were added to a solution of methyl 4-(2-cyclobutyl-1-hydroxyethyl)benzoate (375 mg, 1.6 mmol, 1 equiv.) in 6 mL dichloromethane at 0° C. The reaction mixture was let to reach r.t., and then stirred overnight. Water was added to the reaction mixture and the product was extracted with DCM. The combined organic layers were washed with sat. aq. NaHCO₃, brine, dried over MgSO₄, filtered and concentrated under reduced pressure affording a yellow solid which was used in the next step without further purification (499 mg, 1.6 mmol, 100% yield).

Step C

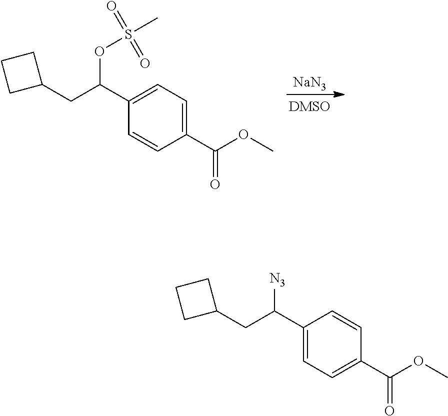 [SMILES: COC(=O)c1ccc(C(CC2CCC2)OS(C)(=O)=O)cc1 ; COC(=O)c1ccc(C(CC2CCC2)N=[N+]=[N-])cc1 ; "NaN3"]

Crude methyl 4-(2-(4-chlorophenyl)-1-((methylsulfonyl)oxy)ethyl)benzoate (499 mg, 1.6 mmol, 1 equiv.) was dissolved in 4 mL DMSO, and sodium azide (1.2 equiv.) was added. The reaction mixture was vigorously stirred at r.t. overnight. Full conversion was observed by LC-MS. The reaction was quenched with water and extracted with ethyl acetate. The organic layer was washed with brine, dried over MgSO₄, filtered and concentrated under reduced pressure. The residue was purified by flash column chromatography (hexane/EtOAc 96:4) to afford desired product as a colorless oil (332 mg, 1.28 mmol, 80% yield).

Step D

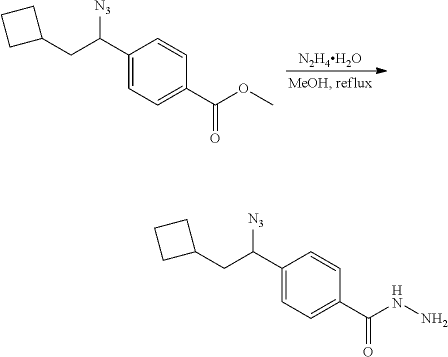 [SMILES: COC(=O)c1ccc(C(CC2CCC2)N=[N+]=[N-])cc1 ; "N2H4·H2O" ; [N-]=[N+]=NC(CC1CCC1)c1ccc(C(=O)NN)cc1]

A solution of methyl 4-(1-azido-2-(4-chlorophenyl)ethyl)benzoate (330 mg, 1.27 mmol, 1 equiv.) in methanol (5 mL) was added to hydrazine monohydrate (5 equiv.) under gentle stirring, dropwise. The mixture was refluxed overnight. Full conversion of methyl ester to hydrazide was observed by LC-MS (and TLC). The reaction mixture was concentrated under reduced pressure and the crude product (330 mg, 1.27 mmol, 100% yield) was used in the next step without further purification.

Step E

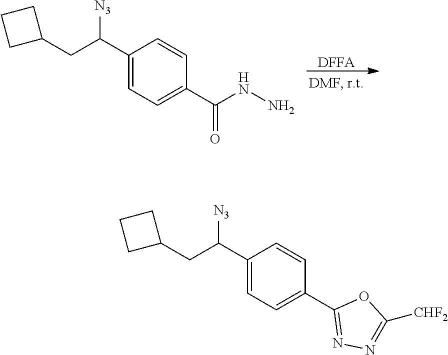 [SMILES: [N-]=[N+]=NC(CC1CCC1)c1ccc(C(=O)NN)cc1 ; "DFFA" ; [N-]=[N+]=NC(CC1CCC1)c1ccc(-c2nnc(C(F)F)o2)cc1]

4-(1-azido-2-cyclobutylethyl)benzohydrazide (330 mg, 1.27 mmol, 1 equiv.) was dissolved in dry DMF (5 mL) under argon. Difluoroacetic anhydride (3 equiv.) was slowly added, keeping temperature below 30° C. (ice/NaCl bath). After completing the addition, the mixture was allowed to reach r.t. The flask was sealed and the reaction mixture was stirred at r.t. overnight. 75% conversion was observed by LC-MS. The reaction mixture was diluted with water, and the product was extracted with ethyl acetate (3×). Combined organic layers were washed with sat. aq. NaHCO₃ and brine, dried over MgSO₄ and evaporated to dryness under reduced pressure. The crude residue was purified by flash chromatography (hexane/EtOAc 96:4 to 8:2) affording the product as a yellow oil (193 mg, 0.6 mmol, 47% yield).

Step F

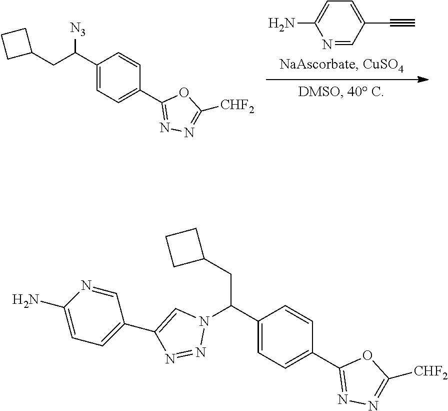

Copper(II) sulfate pentahydrate (0.2 equiv., 0.5 M aqueous solution) and sodium L-ascorbate (0.4 equiv., 1 M aqueous solution) were added to a solution of 2-(4-(1-azido-2-cyclobutylethyl)phenyl)-5-(difluoromethyl)-1,3,4-oxadiazole (75 mg, 0.235 mmol, 1 equiv.) and 5-ethynylpyridin-2-amine (28 mg, 0.235 mmol, 1 equiv.) in 1.4 mL DMSO. The reaction mixture was stirred at 40° C. over 2 h. Full conversion of the starting material was detected by LC-MS. Reaction mixture was filtered through a syringe filter and submitted to prep-HPLC with acidic conditions. After evaporation of fractions, 30 mg of the target compound (0.067 mmol, 29% yield) were obtained as a white solid (m/z 438.19 [MH+]).

The following compounds were synthesized according to the same procedure:

| Compd. | Structure | m/z [MH+] |
|---|---|---|
| 104 | 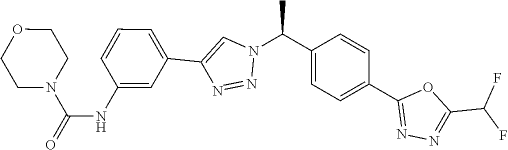 [SMILES: C[C@@H](c1ccc(-c2nnc(C(F)F)o2)cc1)n1cc(-c2cccc(NC(=O)N3CCOCC3)c2)nn1] | 496.22 |
| 165 | 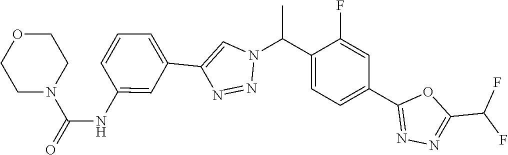 [SMILES: CC(c1ccc(-c2nnc(C(F)F)o2)cc1F)n1cc(-c2cccc(NC(=O)N3CCOCC3)c2)nn1] | 514.6 |
| 184 | 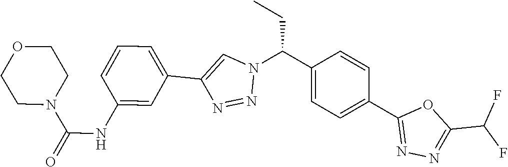 [SMILES: CC[C@H](c1ccc(-c2nnc(C(F)F)o2)cc1)n1cc(-c2cccc(NC(=O)N3CCOCC3)c2)nn1] | 510.19 |
| 185 | 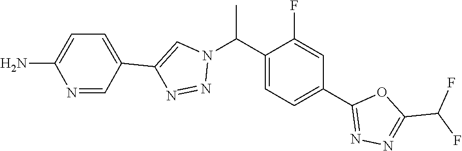 [SMILES: CC(c1ccc(-c2nnc(C(F)F)o2)cc1F)n1cc(-c2ccc(N)nc2)nn1] | 402.5 |
| 186 | 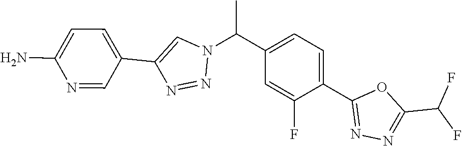 [SMILES: CC(c1ccc(-c2nnc(C(F)F)o2)c(F)c1)n1cc(-c2ccc(N)nc2)nn1] | 402.08 |
| 189 | 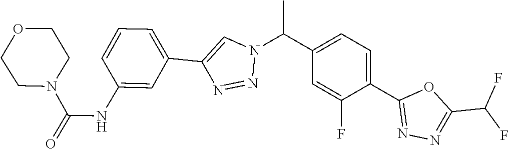 [SMILES: CC(c1ccc(-c2nnc(C(F)F)o2)c(F)c1)n1cc(-c2cccc(NC(=O)N3CCOCC3)c2)nn1] | 514.6 |
| 193 | 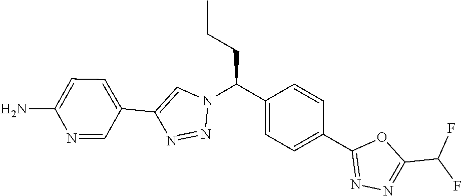 [SMILES: CCC[C@@H](c1ccc(-c2nnc(C(F)F)o2)cc1)n1cc(-c2ccc(N)nc2)nn1] | 411.98 |
| 194 | 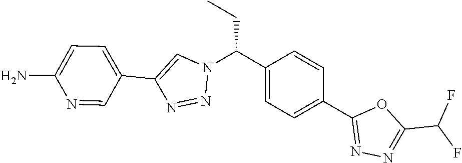 [SMILES: CC[C@H](c1ccc(-c2nnc(C(F)F)o2)cc1)n1cc(-c2ccc(N)nc2)nn1] | 398.1 |
| 205 | 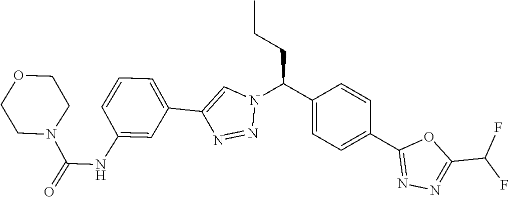 [SMILES: CCC[C@@H](c1ccc(-c2nnc(C(F)F)o2)cc1)n1cc(-c2cccc(NC(=O)N3CCOCC3)c2)nn1] | 524.23 |
| 221 | 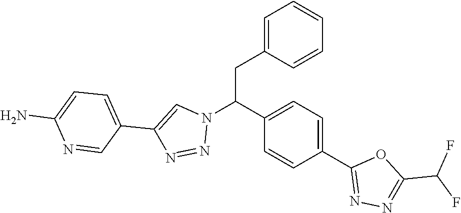 [SMILES: Nc1ccc(-c2cn(C(Cc3ccccc3)c3ccc(-c4nnc(C(F)F)o4)cc3)nn2)cn1] | 460.6 |
| 235 | 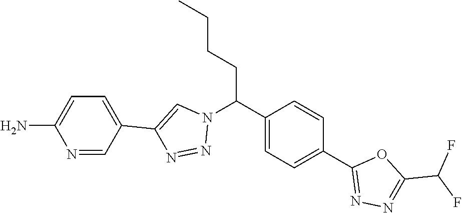 [SMILES: CCCCC(c1ccc(-c2nnc(C(F)F)o2)cc1)n1cc(-c2ccc(N)nc2)nn1] | 426.7 |
| 244 | 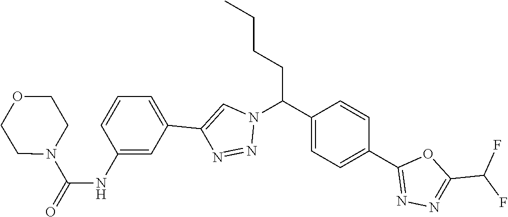 [SMILES: CCCCC(c1ccc(-c2nnc(C(F)F)o2)cc1)n1cc(-c2cccc(NC(=O)N3CCOCC3)c2)nn1] | 538.7 |
| 303 | 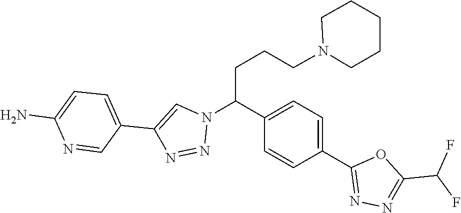 [SMILES: Nc1ccc(-c2cn(C(CCCN3CCCCC3)c3ccc(-c4nnc(C(F)F)o4)cc3)nn2)cn1] | 495.2 |

### Example 46. Synthesis of N-{3-[1-(6-aminopyridin-3-yl)-1H-1,2,3-triazol-4-yl]-3-{4-[5-(difluoromethyl)-1,3,4-oxadiazol-2-yl]phenyl}propyl}methanesulfonamide (Compd. 62)

Step A

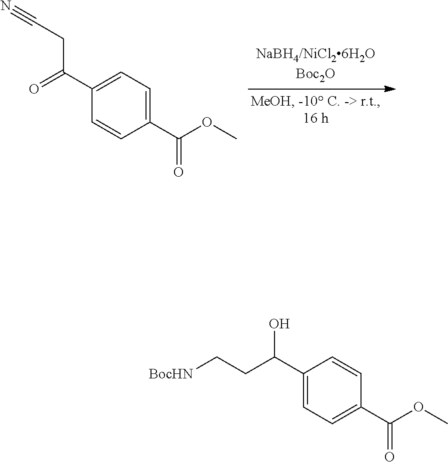 [SMILES: COC(=O)c1ccc(C(=O)CC#N)cc1 ; COC(=O)c1ccc(C(O)CCNC(=O)OC(C)(C)C)cc1]

Methyl 4-(2-cyanoacetyl)benzoate (900 mg, 4.4 mmol, 1 equiv.), di-tert-butyl dicarbonate (2 equiv.) and nickel chloride hexahydrate (0.02 equiv.) were dissolved in 50 mL anhydrous MeOH. The mixture was cooled down to −10° C., and sodium borohydride (7 equiv.) was added in portions. The reaction mixture was stirred at r.t. overnight. The mixture was diluted with ethyl acetate, washed with water and brine, dried over Na₂SO₄ and filtered. Evaporation of volatiles gave a crude product (1.2 g, 3.9 mmol, 87% yield) which was used in subsequent steps without further purification.

Step B

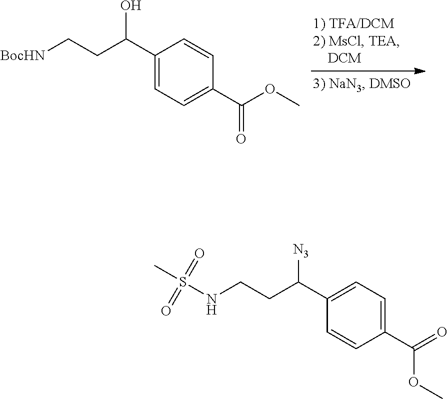

Methyl 4-(3-((tert-butoxycarbonyl)amino)-1-hydroxypropyl)benzoate (600 mg, 1.94 mmol, 1 equiv.) was dissolved in 10 mL DCM. Trifluoroacetic acid (10 equiv.) was added and the solution was stirred at r.t. overnight. Full conversion to the desired deprotected intermediate was observed by LC-MS. The excess of TFA was removed by evaporation.

The residue was dissolved in 10 mL DCM, and triethylamine (5 equiv.) and mesyl chloride (2.5 equiv.) were added. The reaction mixture was stirred at r.t. overnight. The reaction mixture was diluted with DCM, washed with brine (twice), dried over MgSO₄, filtered and concentrated.

The crude intermediate thus obtained was dissolved in 5 mL DMSO, and sodium azide (1.5 equiv.) was added. The reaction mixture was stirred at r.t. over 1 h. The mixture was diluted with MTBE, washed with brine (twice), dried over MgSO₄, filtered and concentrated. Crude product was purified by flash column chromatography (hexane/EtOAc 8:2 to 1:1), obtaining 225 mg of the desired product (0.72 mmol, 37% yield).

Step C

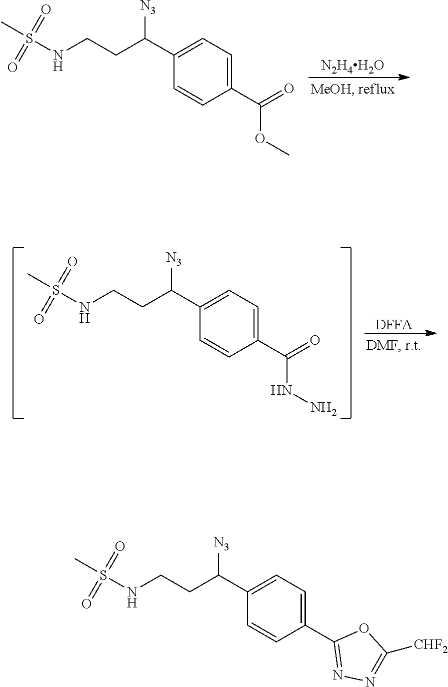

A solution of methyl 4-(1-azido-3-(methylsulfonamido)propyl)benzoate (225 mg, 0.72 mmol, 1 equiv.) in methanol (10 mL) was added to hydrazine monohydrate (5 equiv.) under gentle stirring, dropwise. The mixture was refluxed overnight. Full conversion of methyl ester to hydrazide was observed by LC-MS (and TLC). The reaction mixture was concentrated under reduced pressure. The crude N-(3-azido-3-(4-(hydrazinecarbonyl)phenyl)propyl)methanesulfonamide obtained was dissolved in dry DMF (3 mL) under argon. Difluoroacetic anhydride (2.5 equiv.) was slowly added, keeping temperature below 30° C. (ice/NaCl bath). After addition was complete, the mixture was let to reach r.t. The flask was sealed and the reaction mixture was stirred at r.t. overnight. Full conversion was observed by LC-MS. The reaction mixture was diluted with water, and the product was extracted with ethyl acetate (3×). Combined organic layers were washed with sat. aq. NaHCO₃ and brine, dried over MgSO₄, filtered and evaporated to dryness under reduced pressure. The crude residue was purified by flash chromatography (hexane/EtOAc 8:2 to 1:1) affording the desired product (220 mg, 0.59 mmol, 82% yield).

Step D

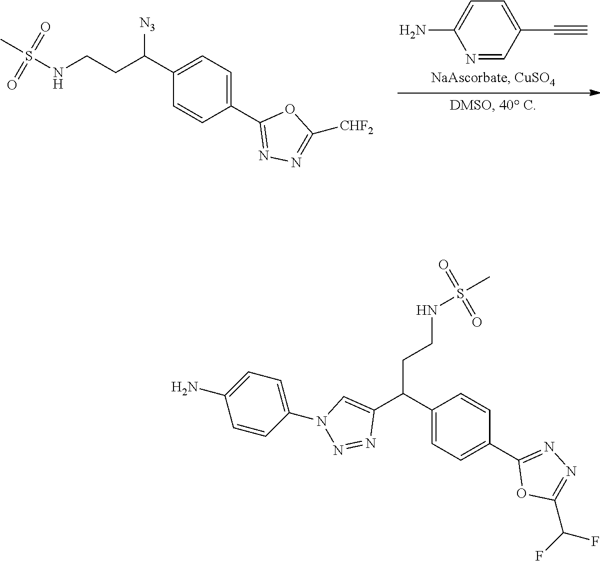

Copper(II) sulfate pentahydrate (0.15 equiv., 0.5 M aqueous solution) and sodium L-ascorbate (0.15 equiv., 1M aq. sol.) were added to a solution of N-[3-azido-3-[4-[5-(difluoromethyl)-1,3,4-oxadiazol-2-yl]phenyl]propyl]methanesulfonamide (46 mg, 0.124 mmol, 1 equiv.) and 5-ethynylpyridin-2-amine (15 mg, 0.124 mmol, 1 equiv.) in 1 mL DMSO. The reaction mixture was stirred at 40° C. over 2 h. Full conversion of the starting material was detected by LC-MS. The reaction mixture was filtered through a syringe filter and submitted to prep-HPLC (neutral conditions). After evaporation of fractions 34 mg of target compound (0.069 mmol, 56% yield) were obtained as a white solid (m/z 491.50 [MH+]).

The following compounds were synthesized according to the same procedure:

| Compd. | Structure | m/z [MH+] |
|---|---|---|
| 310 | 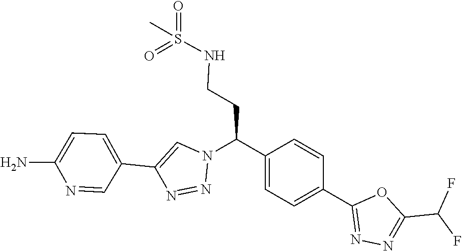 [SMILES: CS(=O)(=O)NCC[C@@H](c1ccc(-c2nnc(C(F)F)o2)cc1)n1cc(-c2ccc(N)nc2)nn1] | 491.05 |
| 311 | 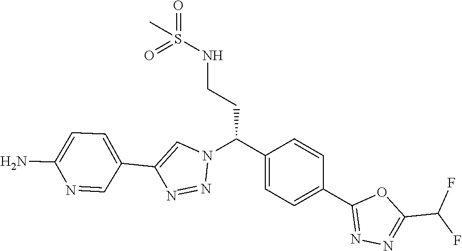 [SMILES: CS(=O)(=O)NCC[C@H](c1ccc(-c2nnc(C(F)F)o2)cc1)n1cc(-c2ccc(N)nc2)nn1] | 491.06 |

### Example 47. Synthesis of 5-(1-(1-(5-(5-(difluoromethyl)-1,3,4-oxadiazol-2-yl)pyridin-2-yl)ethyl)-1H-1,2,3-triazol-4-yl)pyridin-2-amine (Compd. 59), (R)-5-(1-(1-(5-(5-(difluoromethyl)-1,3,4-oxadiazol-2-yl)pyridin-2-yl)ethyl)-1H-1,2,3-triazol-4-yl)pyridin-2-amine (Compd. 32) and (S)-5-(1-(1-(5-(5-(difluoromethyl)-1,3,4-oxadiazol-2-yl)pyridin-2-yl)ethyl)-1H-1,2,3-triazol-4-yl)pyridin-2-amine (Compd. 171)

Step A

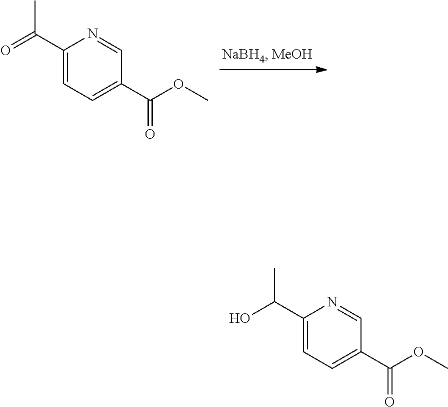

Methyl 6-acetylnicotinate (500 mg, 2.79 mmol, 1 equiv.) was dissolved in 20 mL methanol. Sodium borohydride (1.2 equiv.) was added in portions to the reaction mixture at 0° C. The reaction mixture was stirred over 1 h, following conversion by LC-MS. The reaction was quenched with water and extracted in EtOAc. Collected organic layers were washed with brine, dried over MgSO₄, filtered and concentrated by rotary evaporation. The product was obtained as a yellow oil (345 mg, 1.9 mmol, 68% yield), which was used in the next step without further purification.

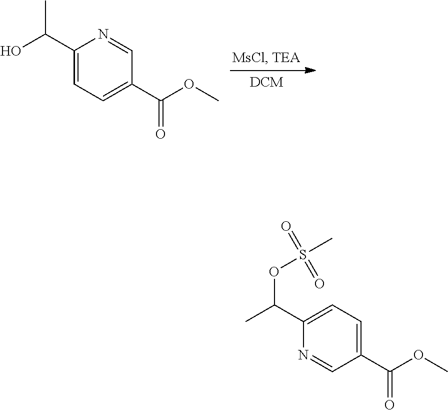 [SMILES: COC(=O)c1ccc(C(C)O)nc1 ; COC(=O)c1ccc(C(C)OS(C)(=O)=O)nc1]

Triethylamine (2 equiv.) and mesyl chloride (1.2 equiv.) were added to a solution of methyl 6-(1-hydroxyethyl)nicotinate (345 mg, 1.9 mmol, 1 equiv.) in 10 mL dichloromethane at 0° C. The reaction mixture was stirred at 0° C. for 30 min, and then allowed to reach r.t. over 4 h. The mixture was then diluted with DCM, washed with water and brine, dried over magnesium sulfate and filtered. Volatiles were removed under reduced pressure, and the product was obtained as a yellow solid (408 mg, 1.57 mmol, 82% yield), which was used in the subsequent step without further purification.

Step C

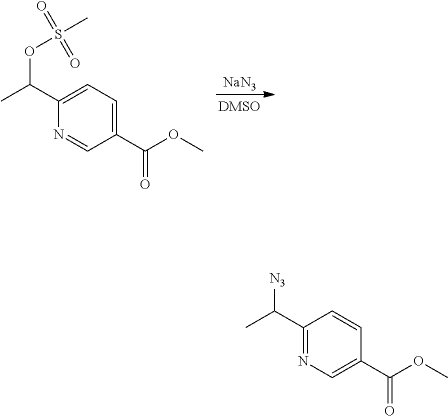 [SMILES: COC(=O)c1ccc(C(C)OS(C)(=O)=O)nc1 ; COC(=O)c1ccc(C(C)N=[N+]=[N-])nc1 ; "NaN3"]

Crude methyl 6-(1-((methylsulfonyl)oxy)ethyl)nicotinate (387 mg, 1.49 mmol, 1 equiv.) was dissolved in 5 mL DMSO, and sodium azide (1.4 equiv.) was added. The reaction mixture was stirred at r.t. overnight. Partial conversion was observed by LC-MS. The reaction was quenched with water and extracted with EtOAc. The organic layer was washed with brine, dried over Na₂SO₄, filtered and concentrated under reduced pressure, affording a yellow oil (248 mg, 1.2 mmol, 80% yield) which was used in the next step without further purification.

Step D

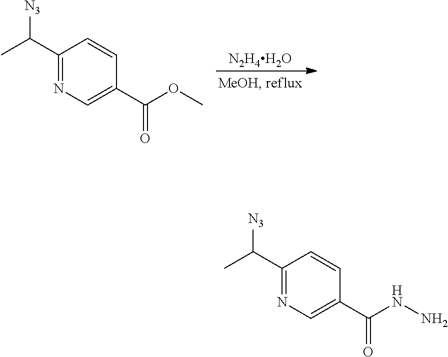

A solution of methyl 6-(1-azidoethyl)nicotinate (190 mg, 0.92 mmol, 1 equiv.) in methanol (5 mL) was added to hydrazine monohydrate (4 equiv.) under gentle stirring, dropwise. Mixture was refluxed overnight. Full conversion of the methyl ester to hydrazide was observed by LC-MS (and TLC). The reaction mixture was concentrated under reduced pressure and the crude product (190 mg, 0.92 mmol, 100% yield) was used for the next step without further purification.

Step E

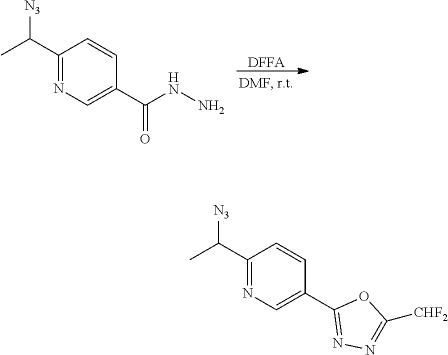 [SMILES: CC(N=[N+]=[N-])c1ccc(C(=O)NN)cn1 ; "DFFA" ; CC(N=[N+]=[N-])c1ccc(-c2nnc(C(F)F)o2)cn1]

6-(1-azidoethyl)nicotinohydrazide (190 mg, 0.92 mmol, 1 equiv.) was dissolved in dry DMF (3 mL) under argon. Difluoroacetic anhydride (3 equiv.) was slowly added, keeping temperature below 30° C. (ice/NaCl bath). After addition was complete the temperature was let to reach r.t. The flask was sealed and the reaction mixture was stirred at r.t. overnight. Full conversion was observed by LC-MS. Sat. aq. NaHCO₃ was added to the reaction mixture to quench difluoroacetic anhydride excess. Then water was added, and the product was extracted with ethyl acetate (3×). Organic layers were collected together, washed with sat. aq. NaHCO₃ and brine, dried over Na₂SO₄ and evaporated to dryness under reduced pressure. The crude residue was purified by flash column chromatography (hexane/EtOAc 85:15) affording the product as a yellow oil (137 mg, 0.51 mmol, 56% yield).

Step F

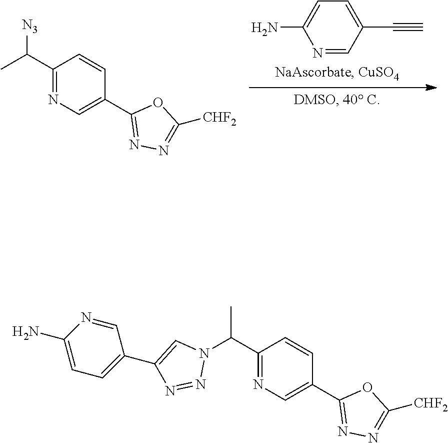

Copper(II) sulfate pentahydrate (0.2 equiv., 0.5 M aqueous solution) and sodium L-ascorbate (0.4 equiv., 1 M aqueous solution) were added to a solution of 2-[6-(1-azidoethyl)pyridin-3-yl]-5-(difluoromethyl)-1,3,4-oxadiazole (80 mg, 0.30 mmol, 1 equiv.) and 5-ethynylpyridin-2-amine (35.5 mg, 0.30 mmol, 1 equiv.) in 1.5 mL DMSO. The reaction mixture was agitated at 40° C. overnight. Full conversion of the starting material was detected by LC-MS. The reaction mixture was diluted with water and extracted in EtOAc. The organic layer was washed with sat. aq. NaHCO₃ and brine, dried over MgSO₄, filtered and concentrated under reduced pressure to afford a yellow solid which was purified by flash column chromatography (hexane/EtOAc 95/5 to 9/1) affording compd. 59 as a beige solid (84 mg, 0.22 mmol, 72% yield, m/z 385.1 [MH+]).

Step G

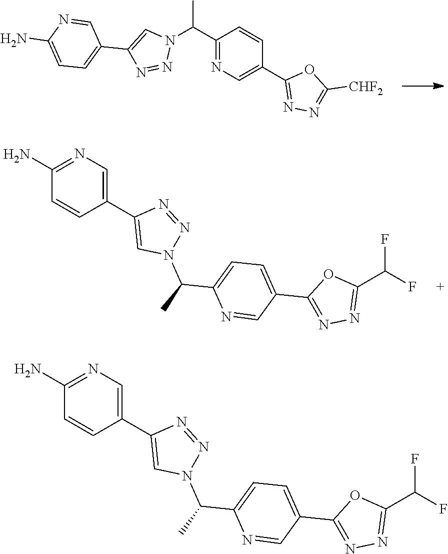 [SMILES: CC(c1ccc(-c2nnc(C(F)F)o2)cn1)n1cc(-c2ccc(N)nc2)nn1 ; C[C@H](c1ccc(-c2nnc(C(F)F)o2)cn1)n1cc(-c2ccc(N)nc2)nn1 ; C[C@@H](c1ccc(-c2nnc(C(F)F)o2)cn1)n1cc(-c2ccc(N)nc2)nn1]

5-(1-(1-(5-(5-(difluoromethyl)-1,3,4-oxadiazol-2-yl)pyridin-2-yl)ethyl)-1H-1,2,3-triazol-4-yl)pyridin-2-amine (compd. 59) was dissolved to 5 mg/mL in EtOH and was then purified by SFC. Combined fractions of each of the enantiomers were then evaporated to dryness by rotary evaporation. The resultant solids were then dried in a vacuum oven at 35° C. and 5 mbar until constant weight to afford pure enantiomers as white solids.

Compd. 32: (25 mg, 0.065 mmol)

Compd. 171: (25 mg, 0.065 mmol)

Compd. 32 was also synthesized by enantiospecific synthesis, confirming its absolute configuration.

The following compounds were prepared according to the same procedure:

| Compd. | Structure | m/z [MH+] |
|---|---|---|
| 66 | 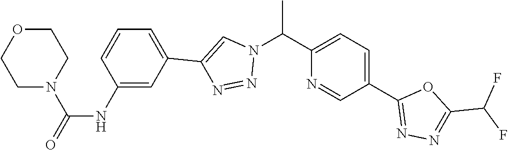 [SMILES: CC(c1ccc(-c2nnc(C(F)F)o2)cn1)n1cc(-c2cccc(NC(=O)N3CCOCC3)c2)nn1] | 497.3 |
| 106 | 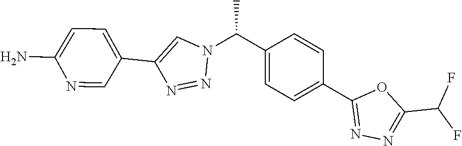 [SMILES: C[C@H](c1ccc(-c2nnc(C(F)F)o2)cc1)n1cc(-c2ccc(N)nc2)nn1] | 484.02 |
| 143 | 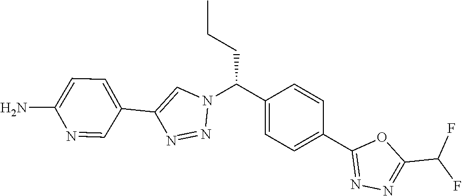 [SMILES: CCC[C@H](c1ccc(-c2nnc(C(F)F)o2)cc1)n1cc(-c2ccc(N)nc2)nn1] | 411.98 |
| 250 | 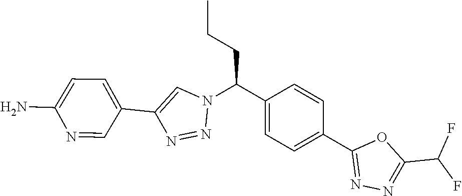 [SMILES: CCC[C@@H](c1ccc(-c2nnc(C(F)F)o2)cc1)n1cc(-c2ccc(N)nc2)nn1] | 411.98 |
| 255 | 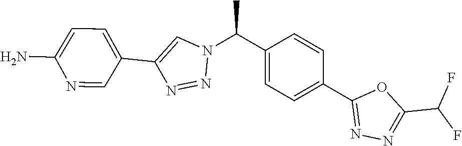 [SMILES: C[C@@H](c1ccc(-c2nnc(C(F)F)o2)cc1)n1cc(-c2ccc(N)nc2)nn1] | 484.02 |

### Example 48. Synthesis of N-(3-(4-(6-aminopyridin-3-yl)-1H-1,2,3-triazol-1-yl)-3-(5-(5-(difluoromethyl)-1,3,4-oxadiazol-2-yl)pyridin-2-yl)propyl)methanesulfonamide (Compd. 293)

Step A

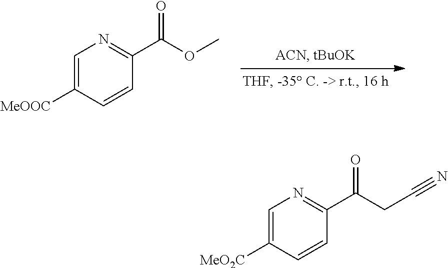

ACN (1.1 equiv.) was added to a solution of potassium tert-butoxide (1.1 equiv.) in 100 mL anhydrous THF at −35° C., and the mixture was stirred for 30 min. Dimethyl pyridine-2,5-dicarboxylate (5 g, 25.6 mmol, 1 equiv.) was added as a suspension in 50 mL anhydrous THF. The reaction mixture was stirred at r.t. overnight. 60% conversion was observed by HPLC. A yellow solid was formed and collected by filtration. The solid obtained was dissolved in water, pH of the solution was adjusted to around 5. The precipitate which formed was filtered and dried (1.5 g, 7.3 mmol, 29% yield). Structure of the product was confirmed by NOESY.

Step B

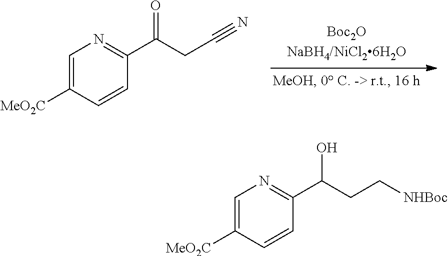 [SMILES: COC(=O)c1ccc(C(=O)CC#N)nc1 ; COC(=O)c1ccc(C(O)CCNC(=O)OC(C)(C)C)nc1]

Methyl 6-(2-cyanoacetyl)nicotinate (1.5 g, 7.3 mmol, 1 equiv.) was dissolved in 80 mL MeOH. The mixture was cooled down to 0° C., and di-tert-butyl-dicarbonate (2 equiv.) and nickel(11) chloride hexahydrate (0.2 equiv.) were added. Then sodium borohydride (7 equiv.) was added in portions. The reaction mixture was stirred at r.t. overnight. The reaction mixture was concentrated, the crude residue was suspended in water and extracted with MTBE. Organic layers were dried over MgSO₄, filtered, concentrated. The obtained crude product was used in the subsequent step without any further purification (2 g, 6.4 mmol, 88% yield).

Step C

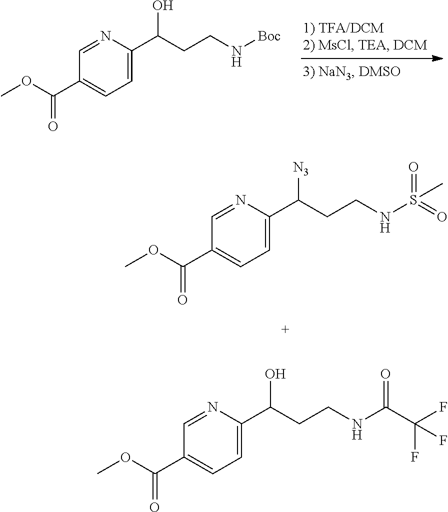

Crude methyl 6-(3-((tert-butoxycarbonyl)amino)-1-hydroxypropyl)nicotinate from the previous step (1 g, 3.2 mmol, 1 equiv.) was dissolved in 15 mL DCM, and TFA (10 equiv.) was added. The reaction mixture was stirred over 2 h. Full conversion was observed by HPLC. The mixture was evaporated to dryness, affording a Boc-deprotected intermediate.

The crude intermediate was dissolved in 10 mL DCM. Triethylamine (4 equiv.) and mesyl chloride (2.5 equiv.) were added, and the resulting mixture was stirred at r.t. overnight. The reaction mixture was diluted with EtOAc and washed with brine. Organic layer was dried over Na₂SO₄, filtered, concentrated.

The crude mesylate intermediate was dissolved in 5 mL DMSO, and sodium azide (1.4 equiv.) was added. The reaction mixture was stirred over 2 h. The reaction mixture was diluted with EtOAc and washed with brine. Organic phase was dried over Na₂SO₄, filtered, evaporated. The crude residue was purified by flash column chromatography (hexane/EtOAc 8:2 to 6:4), isolating two products:

methyl 6-(1-azido-3-(methylsulfonamido)propyl)nicotinate (43 mg, 0.13 mmol, 4% yield)
methyl 6-[1-hydroxy-3-[(2,2,2-trifluoroacetyl)amino]propyl]pyridine-3-carboxylate (110 mg, 0.36 mmol, 11% yield)

Step D

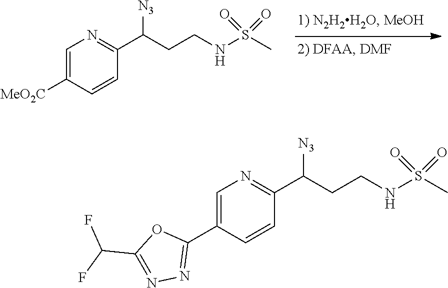 [SMILES: COC(=O)c1ccc(C(CCNS(C)(=O)=O)N=[N+]=[N-])nc1 ; CS(=O)(=O)NCCC(N=[N+]=[N-])c1ccc(-c2nnc(C(F)F)o2)cn1]

Methyl 6-(1-azido-3-(methylsulfonamido)propyl)nicotinate (43 mg, 0.13 mmol, 1 equiv.) was dissolved in 2 mL MeOH, and hydrazine hydrate (5 equiv.) was added. The reaction mixture was refluxed over 2 h under stirring. The reaction mixture was concentrated, and the residue was dissolved in DMF. Difluoroacetic anhydride (3 equiv.) was added, and the reaction mixture was stirred at r.t. for 90 min. Extra 4 equiv. of difluoroacetic anhydride were added, and the mixture was further stirred over 4 h. 50% of the desired product was observed in the mixture. The reaction mixture was diluted with sat. aq. NaHCO₃ and extracted with MTBE. The organic layer was dried over Na₂SO₄, filtered, concentrated. The crude product (41 mg, 0.055 mmol, 40% yield) was used in the next step without any further purification.

Step E

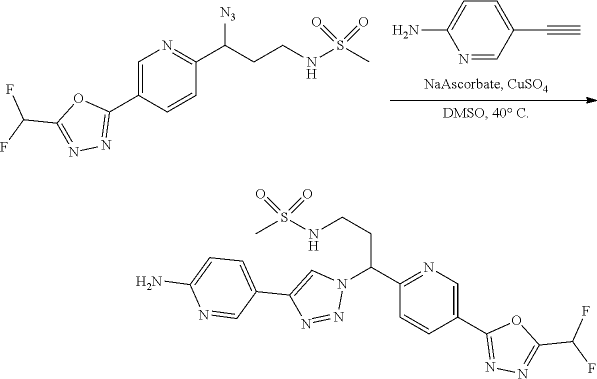

Crude N-[3-azido-3-[5-[5-(difluoromethyl)-1,3,4-oxadiazol-2-yl]pyridin-2-yl]propyl]methanesulfonamide obtained in the previous step (41 mg, 0.055 mmol, 1 equiv.) and 5-ethynylpyridin-2-amine (1 equiv.) were dissolved in 1 mL DMSO. Sodium L-ascorbate (0.15 equiv.) and copper sulfate pentahydrate (0.15 equiv.) were added as solutions in water. The resulting mixture was stirred at r.t. over 3 h. The reaction mixture was submitted to prep-HPLC (ACN/H₂O+0.1% FA) without any workup, obtaining the desired product as a formate salt (3.8 mg, 0.008 mmol, 14% yield, m/z 491.92 [MH+]).

The following compound was prepared according to the same procedure:

| Compd. | Structure | m/z [MH+] |
|---|---|---|
| 295 | 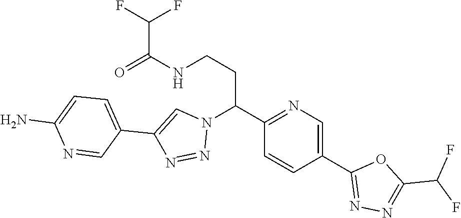 [SMILES: Nc1ccc(-c2cn(C(CCNC(=O)C(F)F)c3ccc(-c4nnc(C(F)F)o4)cn3)nn2)cn1] | 492.28 |

### Example 49. Synthesis of 5-(1-(1-(5-(5-(difluoromethyl)-1,3,4-oxadiazol-2-yl)pyridin-2-yl)-2-(pyrrolidin-1-yl)ethyl)-1H-1,2,3-triazol-4-yl)pyridin-2-amine (Compd. 301)

Step A

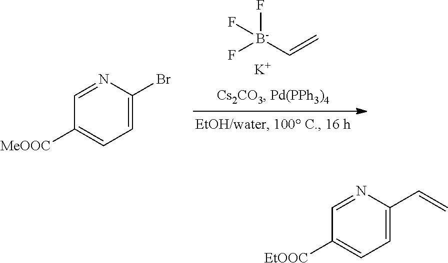

Methyl 6-bromopyridine-3-carboxylate (1.9 g, 8.8 mmol, 1 equiv.), potassium vinyltrifluoroborate (1.8 equiv.) and cesium carbonate (1.9 equiv.) were dissolved in a 4:1 EtOH/water mixture (50 mL). After degassing the mixture with Ar, tetrakis(triphenylphosphine)palladium(0) (0.1 equiv.) was added. The reaction mixture was stirred at 100° C. overnight. Full conversion was observed by HPLC. The white precipitate which formed was filtered off, and the filtrate was diluted with water and extracted with MTBE. The organic layer was dried over Na₂SO₄, filtered, concentrated. Crude ethyl ester product (1.55 g, 8.8 mmol, 100% yield) was used in the next step without any further purification.

Step B

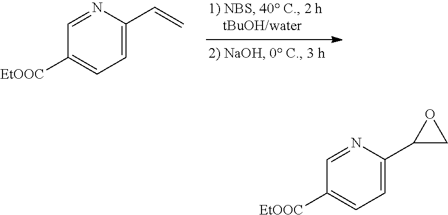

Ethyl 6-ethenylpyridine-3-carboxylate (800 mg, 4.5 mmol, 1 equiv.) was dissolved in a 3:1 tBuOH/water mixture (20 mL), and the resulting mixture was warmed up to 40° C. N-bromosuccinimide (1.5 equiv.) was added and the mixture was stirred at 40° C. over 2 h. Starting material consumption was detected. The reaction mixture was cooled to 0° C., and NaOH (1 equiv.) was added as a solution in water. The resulting mixture was stirred for 3 h, obtaining the desired epoxide. The reaction mixture was diluted with water and the product was extracted into MTBE. The organic phases were collected together, dried over Na₂SO₄, filtered and concentrated. The crude residue was purified by flash column chromatography (hexane/EtOAc 95:5 to 6:4), affording the pure desired product (185 mg, 0.96 mmol, 21% yield).

Step C

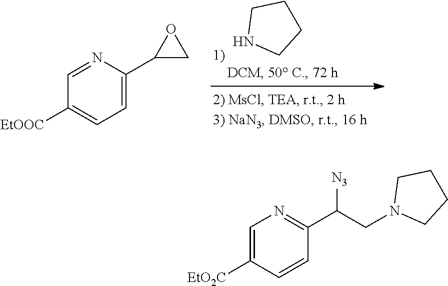 [SMILES: CCOC(=O)c1ccc(C2CO2)nc1 ; C1CCNC1 ; CCOC(=O)c1ccc(C(CN2CCCC2)N=[N+]=[N-])nc1]

Ethyl 6-(oxiran-2-yl)nicotinate (185 mg, 0.96 mmol, 1 equiv.) was dissolved in 4 mL DCM, and pyrrolidine (2.5 equiv.) was added. 3 mL chloroform were added. The reaction mixture was then stirred at 50° C. over 72 h. Full conversion was observed. The mixture was cooled down to 0° C., triethylamine (2 equiv.) and mesyl chloride (2 equiv.) were added. The reaction mixture was stirred at r.t. for 2 h. Full conversion to mesylate intermediate was observed. The mixture was diluted with EtOAc, washed with sat. aq. NaHCO₃, and brine. The organic layer was dried over Na₂SO₄, filtered, concentrated, to give a crude intermediate. The residue was dissolved in 2 mL DMSO and sodium azide was added. The mixture was stirred at r.t. overnight. Full conversion to the desired azide was observed. The mixture was diluted with EtOAc, washed with brine. The organic phase was dried over Na₂SO₄, filtered concentrated. The crude product was purified by flash column chromatography (hexane/EtOAc 8:2 to 2:8), to give pure desired product (180 mg, 0.62 mmol, 65% yield).

Step D

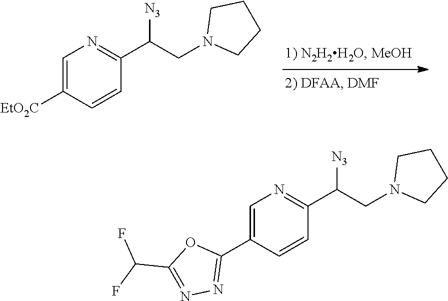 [SMILES: CCOC(=O)c1ccc(C(CN2CCCC2)N=[N+]=[N-])nc1 ; [N-]=[N+]=NC(CN1CCCC1)c1ccc(-c2nnc(C(F)F)o2)cn1]

Ethyl 6-(1-azido-2-(pyrrolidin-1-yl)ethyl)nicotinate (180 mg, 0.62 mmol, 1 equiv.) was dissolved in 5 mL MeOH. Hydrazine hydrate (5 equiv.) was added. The mixture was refluxed over 3 h under stirring. Methanol and hydrazine were removed by evaporation. Intermediate hydrazide was dissolved in 3 mL DMF and difluoroacetic anhydride (4 equiv.) was added. The mixture was stirred at r.t. overnight. The mixture was then diluted with EtOAc and washed with sat. aq. NaHCO₃ and brine. Organic phase was dried over Na₂SO₄, filtered and concentrated to obtain a crude product. Crude was purified by pTLC (hexane/EtOAc 8:2 to 2:8), to give the desired product (34 mg, 0.1 mmol, 16% yield).

Step E

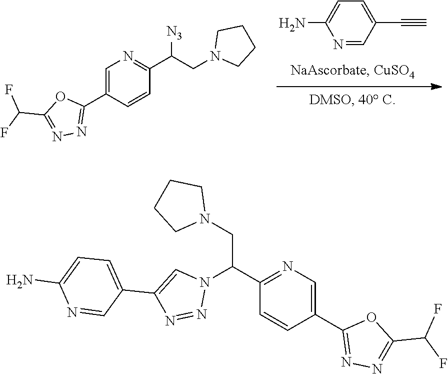

2-(6-(1-azido-2-(pyrrolidin-1-yl)ethyl)pyridin-3-yl)-5-(difluoromethyl)-1,3,4-oxadiazole (34 mg, 0.1 mmol, 1 equiv.) and 5-ethynylpyridin-2-amine (1 equiv.) were dissolved in 0.5 mL DMSO. Sodium ascorbate (0.4 equiv.) and copper sulfate pentahydrate (0.2 equiv.) were added as solutions in water. The resulting mixture was stirred at r.t. over 3 h. The reaction mixture was submitted to prep-HPLC (ACN/H₂O/0.1% FA) without any workup, obtaining the desired product as a bis-formate salt (2.8 mg, 0.006 mmol, 6% yield, m/z 454.11 [MH+]).

### Example 50. Synthesis of N-(3-(3-(4-(5-(difluoromethyl)-1,3,4-oxadiazol-2-yl)-2,6-difluorobenzyl)-1,2,4-oxadiazol-5-yl)phenyl)morpholine-4-carboxamide (Compd. 145)

Step A

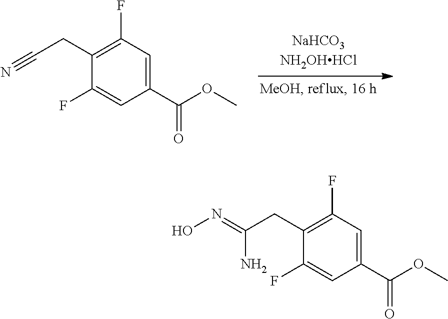 [SMILES: COC(=O)c1cc(F)c(CC#N)c(F)c1 ; COC(=O)c1cc(F)c(C/C(N)=N/O)c(F)c1]

A solution of methyl 4-(cyanomethyl)-3,5-difluorobenzoate (2.1 g, 10 mmol, 1 equiv.) sodium hydrogen carbonate (1.05 equiv.) and hydroxylamine hydrochloride (1.05 equiv.) in 20 mL MeOH was refluxed under stirring overnight. Full conversion was detected by TLC. The reaction mixture was concentrated under reduced pressure. Water and EtOAc were added to the residue. The solid which formed was collected by filtration and rinsed with water and methanol. The precipitated powder was dried under reduced pressure (1.7 g, 7 mmol, 70% yield).

Step B

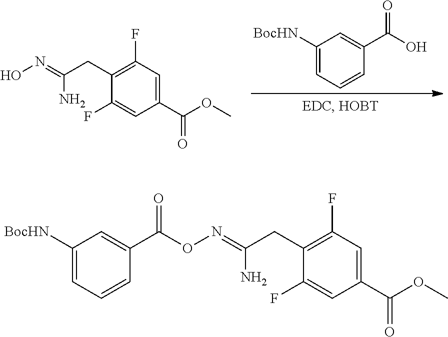 [SMILES: COC(=O)c1cc(F)c(C/C(N)=N/O)c(F)c1 ; CC(C)(C)OC(=O)Nc1cccc(C(=O)O)c1 ; COC(=O)c1cc(F)c(C/C(N)=N/OC(=O)c2cccc(NC(=O)OC(C)(C)C)c2)c(F)c1]

A solution of the 3-((tert-butoxycarbonyl)amino)benzoic acid (1 equiv.), EDC (1.1 equiv.) and HOBt (1.05 equiv.) in 8 mL DMF was stirred at r.t. over 1 h. The amidoxime obtained in step A (515 mg, 2.1 mmol, 1 equiv.) was added. The reaction mixture was stirred 4 h. Full conversion to product was detected by HPLC. The reaction mixture was diluted with water. The white solid formed was washed with water and dried on air (862 mg, 1.86 mmol, 88% yield).

Step C

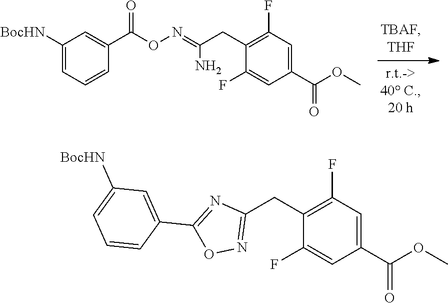 [SMILES: COC(=O)c1cc(F)c(C/C(N)=N/OC(=O)c2cccc(NC(=O)OC(C)(C)C)c2)c(F)c1 ; COC(=O)c1cc(F)c(Cc2noc(-c3cccc(NC(=O)OC(C)(C)C)c3)n2)c(F)c1]

Tetrabutylammonium fluoride (2.4 equiv.) was added in portions to a solution of methyl 4-(2-amino-2-(((3-((tert-butoxycarbonyl)amino)benzoyl)oxy)imino)ethyl)-3,5-difluorobenzoate (862 mg, 1.86 mmol, 1 equiv.) in THF. The reaction mixture was stirred at r.t. over 18 h, and heated to 40° C. for 2 h. Full conversion was observed by TLC (DCM/MeOH 95:5). The reaction mixture was diluted with water and MTBE. Organic layers were washed with water (3 times) and brine, dried over MgSO₄, evaporated and dried in vacuum to give target compound as light-yellow solid. The crude residue was used in next step without purification (735 mg, 1.65 mmol, 89% yield).

Step D

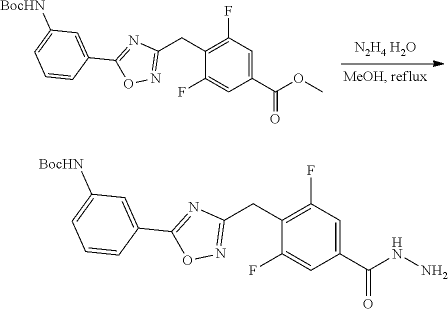 [SMILES: COC(=O)c1cc(F)c(Cc2noc(-c3cccc(NC(=O)OC(C)(C)C)c3)n2)c(F)c1 ; "N2H4 H2O" ; CC(C)(C)OC(=O)Nc1cccc(-c2nc(Cc3c(F)cc(C(=O)NN)cc3F)no2)c1]

A solution of methyl 4-((5-(3-((tert-butoxycarbonyl)amino)phenyl)-1,2,4-oxadiazol-3-yl)methyl)-3,5-difluorobenzoate (735 mg, 1.65 mmol, 1 equiv.) and hydrazine hydrate (15 equiv.) in 20 mL MeOH was stirred under reflux overnight. Full conversion was detected by LC-MS. The reaction mixture was concentrated to dryness under vacuum to obtain pure target compound as a white solid (685 mg, 1.54 mmol, 93% yield). Step E

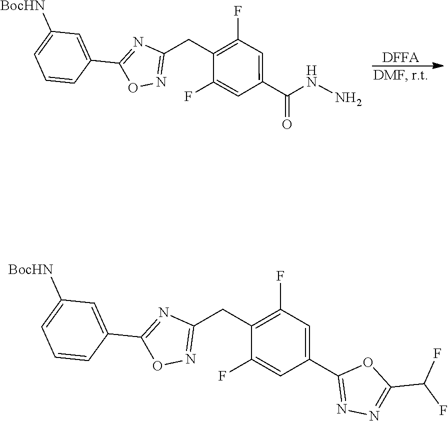 [SMILES: CC(C)(C)OC(=O)Nc1cccc(-c2nc(Cc3c(F)cc(C(=O)NN)cc3F)no2)c1 ; CC(C)(C)OC(=O)Nc1cccc(-c2nc(Cc3c(F)cc(-c4nnc(C(F)F)o4)cc3F)no2)c1]

Difluoroacetic anhydride (4 equiv.) was added to a solution of tert-butyl-(3-(3-(2,6-difluoro-4-(hydrazinecarbonyl)benzyl)-1,2,4-oxadiazol-5-yl)phenyl)carbamate (685 mg, 1.54 mmol, 1 equiv.) in 5 mL DMF at 0° C. The reaction mixture was heated to 70° C. and stirred over 5 h. Then, the mixture was allowed to reach r.t. and stirred overnight. Conversion was confirmed by LC-MS. The reaction mixture was concentrated under reduced pressure and the residue was purified by flash column chromatography (DCM/EtOAc 97:3 to 95:5) to obtain product (80 mg, 0.16 mmol, 10% yield).

Step F

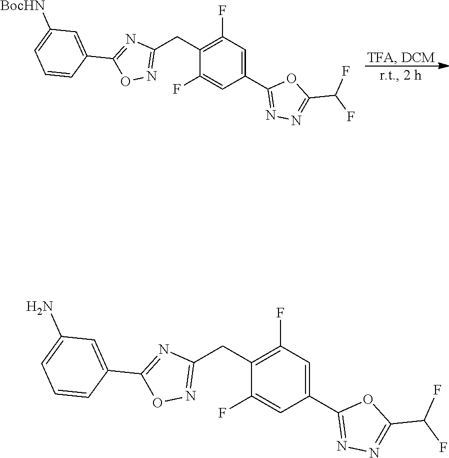 [SMILES: CC(C)(C)OC(=O)Nc1cccc(-c2nc(Cc3c(F)cc(-c4nnc(C(F)F)o4)cc3F)no2)c1 ; Nc1cccc(-c2nc(Cc3c(F)cc(-c4nnc(C(F)F)o4)cc3F)no2)c1]

Tert-butyl (3-(3-(4-(5-(difluoromethyl)-1,3,4-oxadiazol-2-yl)-2,6-difluorobenzyl)-1,2,4-oxadiazol-5-yl)phenyl)carbamate (80 mg, 0.16 mmol, 1 equiv.) was dissolved in 3 mL DCM and trifluoroacetic acid (10 equiv.) was added. The reaction mixture was stirred at r.t. over 2 h, monitoring conversion by TLC. The mixture was diluted with EtOAc, washed with sat. aq. NaHCO₃ and brine, dried over Na₂SO₄, filtered, concentrated and dried in vacuum to give 61 mg of product (0.15 mmol, 95% yield).

Step G

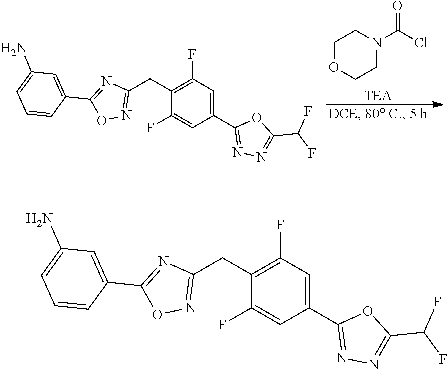 [SMILES: Nc1cccc(-c2nc(Cc3c(F)cc(-c4nnc(C(F)F)o4)cc3F)no2)c1 ; O=C(Cl)N1CCOCC1 ; Nc1cccc(-c2nc(Cc3c(F)cc(-c4nnc(C(F)F)o4)cc3F)no2)c1]

Morpholine-4-carbonyl chloride (2.5 equiv.) and triethylamine (4 equiv.) were added to a solution of 3-(3-(4-(5-(difluoromethyl)-1,3,4-oxadiazol-2-yl)-2,6-difluorobenzyl)-1,2,4-oxadiazol-5-yl)aniline (61 mg, 0.15 mmol, 1 equiv.) in 2 mL DCE. The reaction mixture was stirred at 80° C. over 5 h. Conversion was checked by LC-MS. The mixture was diluted with EtOAc, washed with sat. aq. NaHCO₃ and brine, dried over MgSO₄, evaporated and dried under vacuum. The residue was submitted for prep-HPLC. After evaporation of product containing fractions 22 mg of the target compound were obtained (0.043 mmol, 28% yield, m/z 519.13 [MH+]).

The following compounds were synthesized according to the same procedure:

| | | m/z |
|---|---|---|
| Compd. | Structure | [MH+] |
| 100 | 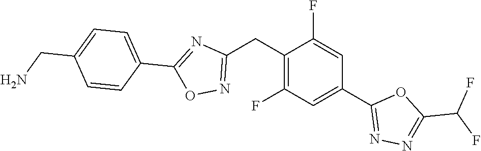 [SMILES: NCc1ccc(-c2nc(Cc3c(F)cc(-c4nnc(C(F)F)o4)cc3F)no2)cc1] | 420 |
| 141 | 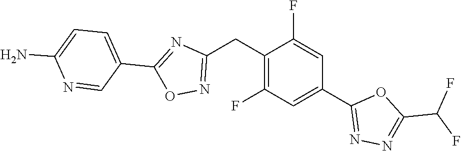 [SMILES: Nc1ccc(-c2nc(Cc3c(F)cc(-c4nnc(C(F)F)o4)cc3F)no2)cn1] | 407.12 |
| 191 | 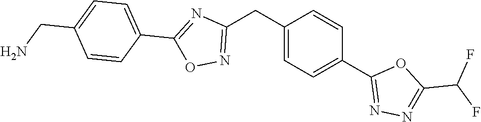 [SMILES: NCc1ccc(-c2nc(Cc3ccc(-c4nnc(C(F)F)o4)cc3)no2)cc1] | 425.14 * |
| 206 | 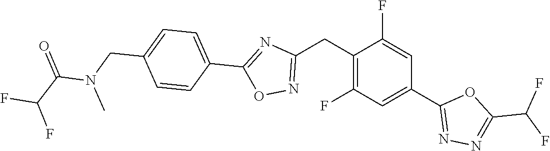 [SMILES: CN(Cc1ccc(-c2nc(Cc3c(F)cc(-c4nnc(C(F)F)o4)cc3F)no2)cc1)C(=O)C(F)F] | 552.97 * |
| 228 | 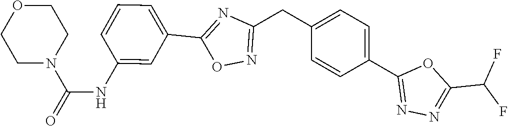 [SMILES: O=C(Nc1cccc(-c2nc(Cc3ccc(-c4nnc(C(F)F)o4)cc3)no2)c1)N1CCOCC1] | 483.2 |
| 247 | 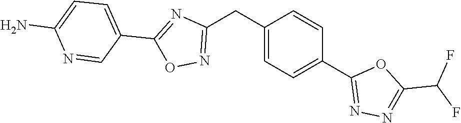 [SMILES: Nc1ccc(-c2nc(Cc3ccc(-c4nnc(C(F)F)o4)cc3)no2)cn1] | 412.02 * |
| 254 | 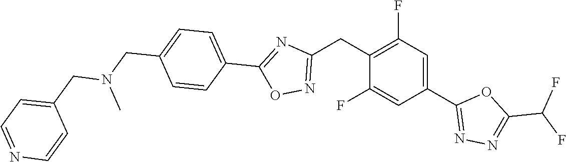 [SMILES: CN(Cc1ccncc1)Cc1ccc(-c2nc(Cc3c(F)cc(-c4nnc(C(F)F)o4)cc3F)no2)cc1] | 525.2 |
| 259 | 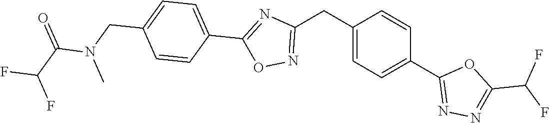 [SMILES: CN(Cc1ccc(-c2nc(Cc3ccc(-c4nnc(C(F)F)o4)cc3)no2)cc1)C(=O)C(F)F] | 517.15 * |
| 262 | 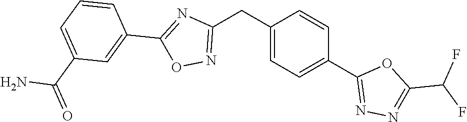 [SMILES: NC(=O)c1cccc(-c2nc(Cc3ccc(-c4nnc(C(F)F)o4)cc3)no2)c1] | 397.97 |

*[M+ACN+H]⁺ was observed.

### Example 51. Synthesis 3-(5-(4-(5-(difluoromethyl)-1,3,4-oxadiazol-2-yl)benzyl)-1,2,4-oxadiazol-3-yl)benzamide (Compd. 226)

Step A

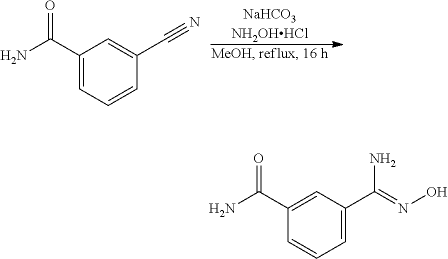 [SMILES: N#Cc1cccc(C(N)=O)c1 ; NC(=O)c1cccc(/C(N)=N/O)c1]

A solution of 3-cyanobenzamide (1 g, 6.8 mmol, 1 equiv.), sodium hydrogen carbonate (2 equiv.) and hydroxylamine hydrochloride (2 equiv.) in 15 mL MeOH was refluxed under stirring overnight. Conversion was monitored by LC-MS. The reaction mixture was filtered and concentrated under reduced pressure. The white solid obtained was used in the next reaction without further purification (940 mg, 5.2 mmol, 76% yield).

Step B

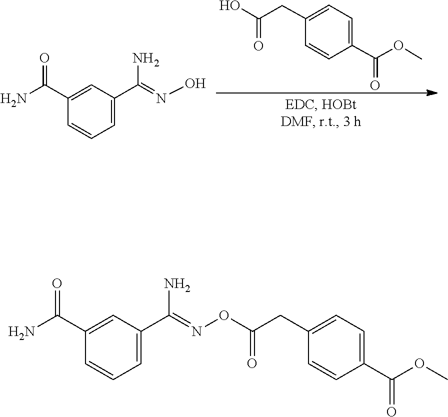 [SMILES: NC(=O)c1cccc(/C(N)=N/O)c1 ; COC(=O)c1ccc(CC(=O)O)cc1 ; COC(=O)c1ccc(CC(=O)O/N=C(\N)c2cccc(C(N)=O)c2)cc1]

A solution of 2-(4-(methoxycarbonyl)phenyl)acetic acid (250 mg, 1.2 mmol, 1 equiv.), EDC (1.2 equiv.) and HOBt (1.1 equiv.) in 5 mL DMF was stirred at r.t. over 1 h. The amidoxime obtained in step A (230 mg, 1.2 mmol, 1 equiv.) was added. The reaction mixture was stirred 2 h. Full conversion to product was detected by LC-MS. The reaction mixture was diluted with EtOAc, washed with sat. aq. NaHCO₃ and brine, dried and evaporated in vacuum to get pure target compound (213 mg, 0.6 mmol, 46% yield).

Step C

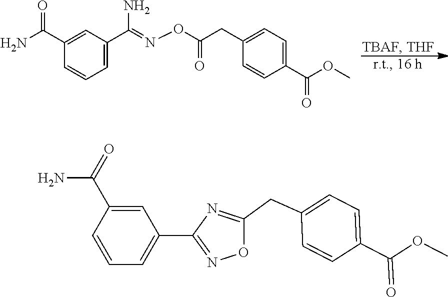 [SMILES: COC(=O)c1ccc(CC(=O)O/N=C(\N)c2cccc(C(N)=O)c2)cc1 ; COC(=O)c1ccc(Cc2nc(-c3cccc(C(N)=O)c3)no2)cc1]

Tetrabutylammonium fluoride (1.5 equiv) was added in portions to a solution of methyl (Z)-4-(2-(((amino(3-carbamoylphenyl)methylene)amino)oxy)-2-oxoethyl)benzoate (213 mg, 0.6 mmol, 1 equiv.) in 8 mL THF. The reaction mixture was stirred at r.t. overnight. Full conversion was observed by TLC. The reaction mixture was diluted with EtOAc, washed with water, sat. aq. NaHCO₃ and brine. Organic layers were dried over Na₂SO₄, filtered and concentrated under vacuum. The residue was purified by flash column chromatography (DCM/MeOH 98:2 to 9:1) to give enough pure target compound (77 mg, 0.23 mmol, 38% yield).

Step D

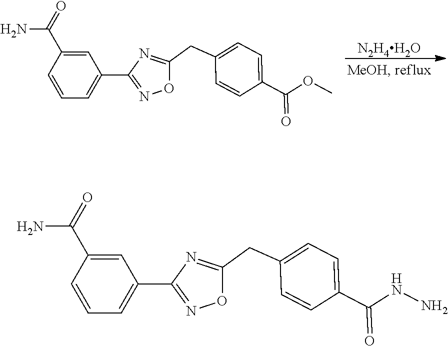 [SMILES: COC(=O)c1ccc(Cc2nc(-c3cccc(C(N)=O)c3)no2)cc1 ; "N2H4·H2O" ; NNC(=O)c1ccc(Cc2nc(-c3cccc(C(N)=O)c3)no2)cc1]

A solution of methyl 4-((3-(3-carbamoylphenyl)-1,2,4-oxadiazol-5-yl)methyl)benzoate (77 mg, 0.23 mmol, 1 equiv.) and hydrazine hydrate (5 equiv.) in 5 mL MeOH was stirred at reflux overnight. Full conversion was detected by LC-MS. The reaction mixture was concentrated. The residue was suspended in acetonitrile and evaporated twice to afford the desired product, which was dried under vacuum (77 mg, 0.023 mmol, 100% yield).

Step E

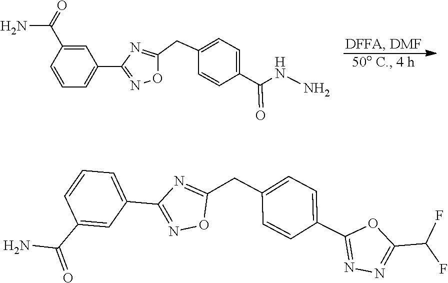 [SMILES: NNC(=O)c1ccc(Cc2nc(-c3cccc(C(N)=O)c3)no2)cc1 ; NC(=O)c1cccc(-c2noc(Cc3ccc(-c4nnc(C(F)F)o4)cc3)n2)c1]

Difluoroacetic anhydride (3 equiv.) was added to a solution of 3-(5-(4-(hydrazinecarbonyl)benzyl)-1,2,4-oxadiazol-3-yl)benzamide (77 mg, 0.023 mmol, 1 equiv.) in 2 mL DMF at 0° C. The reaction mixture was heated to 50° C. and stirred over 4 h. Full conversion was observed by LC-MS. The reaction mixture was diluted with EtOAc, washed with sat. aq. NaHCO₃, water and brine, dried over MgSO₄, filtered and concentrated in vacuum. The residue was purified by prep-HPLC to give target compound (15 mg, 0.036 mmol, 16% yield, m/z 397.89 [MH+]).

The following compound was synthesized according to the same procedure:

| Compd. | Structure | m/z [MH+] |
|---|---|---|
| 135 | 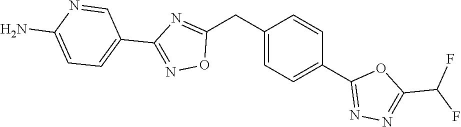 [SMILES: Nc1ccc(-c2noc(Cc3ccc(-c4nnc(C(F)F)o4)cc3)n2)cn1] | 370.94 |
| 375 | 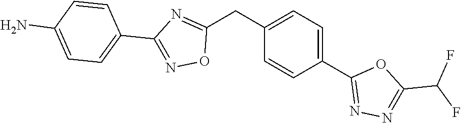 [SMILES: Nc1ccc(-c2noc(Cc3ccc(-c4nnc(C(F)F)o4)cc3)n2)cc1] | 370.19 |
| 379 | 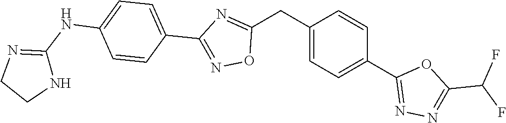 [SMILES: FC(F)c1nnc(-c2ccc(Cc3nc(-c4ccc(NC5=NCCN5)cc4)no3)cc2)o1] | 438.40 |
| 383 | 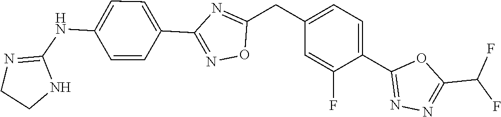 [SMILES: Fc1cc(Cc2nc(-c3ccc(NC4=NCCN4)cc3)no2)ccc1-c1nnc(C(F)F)o1] | 456.16 |
| 408 | 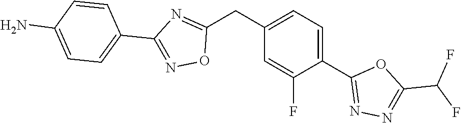 [SMILES: Nc1ccc(-c2noc(Cc3ccc(-c4nnc(C(F)F)o4)c(F)c3)n2)cc1] | 388.19 |

### Example 52. Synthesis of 5-(5-((4-(5-(difluoromethyl)-1,3,4-oxadiazol-2-yl)phenyl)difluoromethyl)-1,2,4-oxadiazol-3-yl)pyridin-2-amine (Compd. 243)

Step A

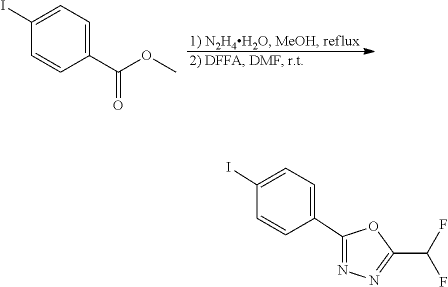

Methyl 4-iodobenzoate (5 g, 19.3 mmol, 1 equiv.) was dissolved in MeOH (5 mL), then hydrazine monohydrate was added (5 equiv.) under stirring. Mixture was stirred at 70° C. overnight. Full conversion of methyl ester to hydrazide was observed by LC-MS (and TLC). The reaction mixture was concentrated under reduced pressure. The residue was diluted with water and extracted with ethyl acetate. The organic phase was washed with sat. aq. NaHCO₃, brine, dried, filtered and concentrated under reduced pressure. 4.37 g (16.2 mmol) of the intermediate hydrazide were obtained. The crude intermediate was dissolved in dry DMF (3 mL) under argon. Difluoroacetic anhydride (4 equiv.) was slowly added, keeping temperature below 30° C. (ice/NaCl bath). After addition was complete the temperature was let to reach r.t. The flask was sealed and the reaction mixture was stirred at 70° C. over 3 h. Full conversion was observed by LC-MS, 50% of the desired product formed.

The reaction mixture was diluted with water forming a white precipitate which was collected by filtration, rinsed with water and dried on air overnight. The obtained solid was suspended in 60 mL chloroform, filtered and rinsed twice with more chloroform. The filtrate was concentrated and the residue was dried in vacuo (3.5 g, 9.7 mmol, 50% yield).

Step B

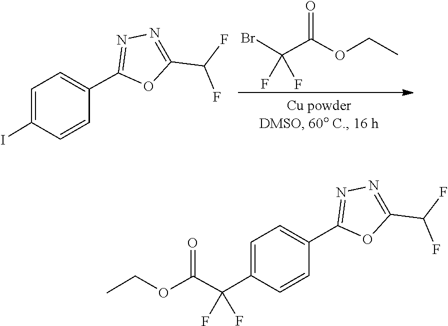

Copper powder (2.6 equiv.) was stirred in 0.1 M HCl for 10 min and then filtered. This procedure was repeated with water, methanol and acetone. The powder was dried in vacuum for 10 min and added to a solution of 2-(difluoromethyl)-5-(4-iodophenyl)-1,3,4-oxadiazole (500 mg, 1.55 mmol, 1 equiv.) and ethyl bromodifluoroacetate (1 equiv.) in DMSO (6 mL). The reaction mixture was stirred at 60° C. overnight. LC-MS confirmed full conversion to product. The mixture was diluted with EtOAc, filtered, washed with water (2 times), sat. aq. NaHCO₃ (2 times) and brine, dried and evaporated in vacuum. The residue was purified by flash chromatography (hexane/EtOAc 9:1 to 8:2) to give pure target product (367 mg, 1.15 mmol, 74% yield).

Step C

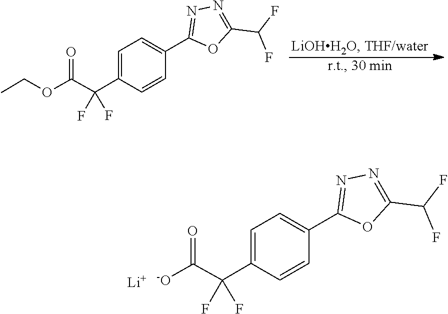

Ethyl 2-(4-(5-(difluoromethyl)-1,3,4-oxadiazol-2-yl)phenyl)-2,2-difluoroacetate (150 mg, 0.47 mmol, 1 equiv.) and lithium hydroxide monohydrate were dissolved in a 2:1 mixture of THF and water. The resulting mixture was stirred at r.t. over 30 min. Full conversion was detected by TLC (eluent DCM/MeOH 98:2). The reaction mixture was evaporated, suspended again in acetonitrile and concentrated. The residue obtained was used without purification in the next step (139 mg, 0.46 mmol, 99% yield).

Step D

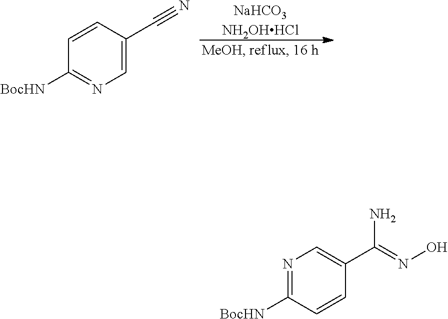 [SMILES: CC(C)(C)OC(=O)Nc1ccc(C#N)cn1 ; CC(C)(C)OC(=O)Nc1ccc(/C(N)=N/O)cn1]

A solution of tert-butyl (5-cyanopyridin-2-yl)carbamate (853 mg, 3.9 mmol, 1 equiv.), sodium hydrogen carbonate (1.1 equiv.) and hydroxylamine hydrochloride (1.1 equiv.) in 10 mL methanol was refluxed under stirring overnight. Conversion was monitored by LC-MS. The reaction mixture was filtered and concentrated under reduced pressure. The residue was suspended in acetonitrile and evaporated twice. The white solid obtained was used in the next step without further purification (978 mg, 3.87 mmol, 99% yield).

Step E

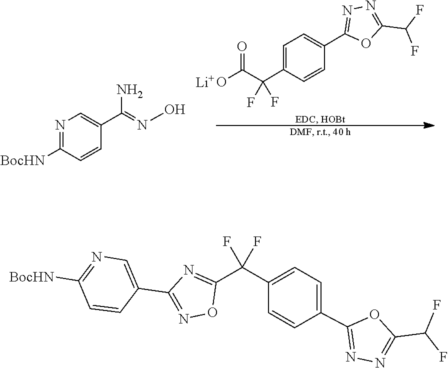 [SMILES: CC(C)(C)OC(=O)Nc1ccc(/C(N)=N/O)cn1 ; [Li+] ; O=C(O)C(F)(F)c1ccc(-c2nnc(C(F)F)o2)cc1 ; CC(C)(C)OC(=O)Nc1ccc(-c2noc(C(F)(F)c3ccc(-c4nnc(C(F)F)o4)cc3)n2)cn1]

A solution of lithium 2-(4-(5-(difluoromethyl)-1,3,4-oxadiazol-2-yl)phenyl)-2,2-difluoroacetate obtained in step C (37 mg, 0.125 mmol, 1 equiv.), EDC (2.2 equiv.) and HOBt (1.1 equiv.) in 1 mL DMF was stirred at r.t. over 15 min. The amidoxime obtained in step D (31 mg, 0.125 mmol, 1 equiv.) was added to the reaction mixture, which was stirred over 40 h. Full conversion to product was detected by LC-MS. The reaction mixture was diluted with EtOAc, washed with sat. aq. NaHCO₃ and brine, dried and evaporated under vacuum to get target compound (38 mg, 0.075 mmol, 60% yield). The crude residue was used in the subsequent step without further purification.

Step F

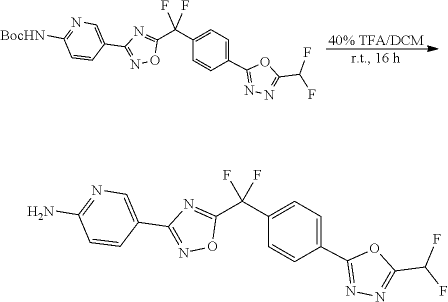 [SMILES: CC(C)(C)OC(=O)Nc1ccc(-c2noc(C(F)(F)c3ccc(-c4nnc(C(F)F)o4)cc3)n2)cn1 ; Nc1ccc(-c2noc(C(F)(F)c3ccc(-c4nnc(C(F)F)o4)cc3)n2)cn1]

tert-butyl (5-(5-((4-(5-(difluoromethyl)-1,3,4-oxadiazol-2-yl)phenyl)difluoromethyl)-1,2,4-oxadiazol-3-yl)pyridin-2-yl)carbamate (38 mg, 0.075 mmol, 1 equiv.) was dissolved in a 40% solution of TFA in DCM (850 μL), and the resulting solution was stirred at r.t. overnight. The reaction mixture was diluted with EtOAc, washed with sat. aq. NaHCO₃ twice and with brine, dried over Na₂SO₄, evaporated and submitted for prep-HPLC. After evaporation of product containing fractions, 5.8 mg of the target compound were obtained (0.014 mmol, 19% yield, m/z 448.14 [M+H+ACN]+).

### Example 53. Synthesis of 5-(5-(4-(5-(difluoromethyl)-1,3,4-oxadiazol-2-yl)benzyl)-1,3,4-oxadiazol-2-yl)pyridin-2-amine (Compd. 218)

Step A

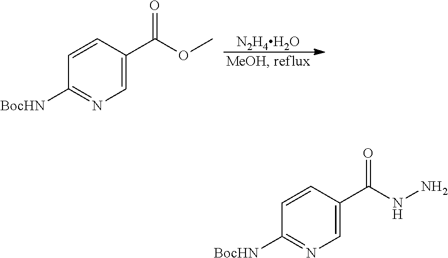 [SMILES: COC(=O)c1ccc(NC(=O)OC(C)(C)C)nc1 ; CC(C)(C)OC(=O)Nc1ccc(C(=O)NN)cn1]

A solution of methyl 6-((tert-butoxycarbonyl)amino)nicotinate (1 g, 3.9 mmol, 1 equiv.) and hydrazine hydrate (5 equiv.) in 20 mL MeOH was stirred at 70° C. overnight. Full conversion was detected by TLC (DCM/MeOH 95:5). The reaction mixture was concentrated to dryness. The residue was resuspended in acetonitrile and evaporated again to yield pure target compound (1 g, 3.9 mmol, 100% yield).

Step B

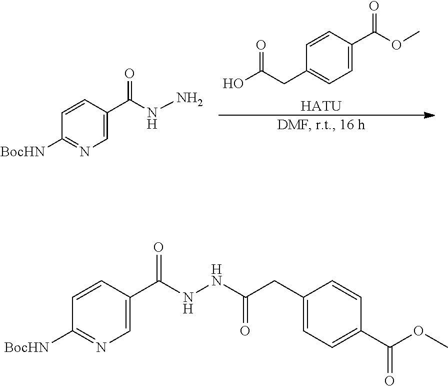

A mixture of 2-(4-(methoxycarbonyl)phenyl)acetic acid (766 mg, 3.9 mmol, 1 equiv.) and HATU (1.5 equiv.) in 4 mL DMF was stirred at r.t. for 10 min. Then hydrazide obtained in the previous step (1 equiv.) was added and the resulting mixture was stirred at r.t. overnight. The mixture was diluted with water and extracted with ethyl acetate. The organic layer was washed with 1M HCl, sat. aq. NaHCO₃, brine, dried over MgSO₄, filtered and concentrated under reduced pressure. The beige crude residue obtained (almost 1:1 mixture of product and a byproduct) was used directly in the next step without any further purification.

Step C

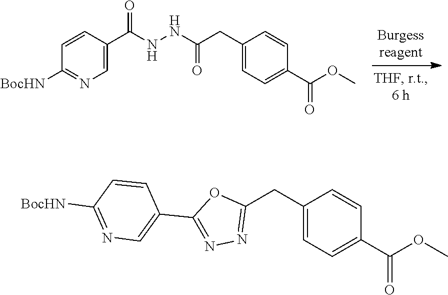 [SMILES: COC(=O)c1ccc(CC(=O)NNC(=O)c2ccc(NC(=O)OC(C)(C)C)nc2)cc1 ; COC(=O)c1ccc(Cc2nnc(-c3ccc(NC(=O)OC(C)(C)C)nc3)o2)cc1]

Methyl 4-(2-(2-(6-((tert-butoxycarbonyl)amino)nicotinoyl)hydrazineyl)-2-oxoethyl)benzoate (1.1 g, 2.56 mmol, 1 equiv.) was dissolved in 10 mL THF. Burgess reagent (2.5 equiv.) was added in portions to the stirring mixture at r.t. over 6 h. The reaction mixture was then diluted with EtOAc, washed 4 times with sat. aq. NaHCO₃ and once with brine, dried over MgSO₄, filtered and evaporated under vacuum. The residue thus obtained was purified by flash column chromatography to give 300 mg of target compound as white solid (0.73 mmol, 28% yield).

Step D

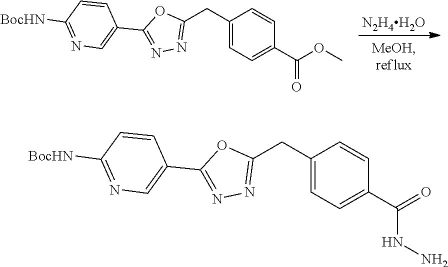

A solution of methyl 4-((5-(6-((tert-butoxycarbonyl)amino)pyridin-3-yl)-1,3,4-oxadiazol-2-yl)methyl)benzoate (150 mg, 0.365 mmol, 1 equiv.) and hydrazine hydrate (15 equiv.) in 10 mL MeOH was stirred under reflux overnight. Full conversion was detected by LC-MS. The reaction mixture was concentrated to dryness under vacuum to obtain pure target compound as a white solid (150 mg, 0.365 mmol, 100% yield).

Step E

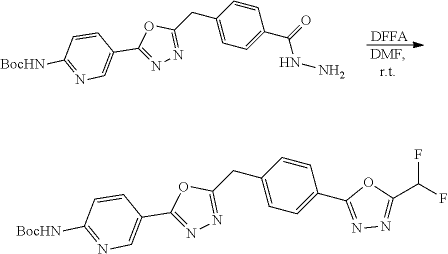 [SMILES: CC(C)(C)OC(=O)Nc1ccc(-c2nnc(Cc3ccc(C(=O)NN)cc3)o2)cn1 ; "DFFA" ; "DMF, r.t." ; CC(C)(C)OC(=O)Nc1ccc(-c2nnc(Cc3ccc(-c4nnc(C(F)F)o4)cc3)o2)cn1]

Difluoroacetic anhydride (3 equiv.) was added to a solution of tert-butyl (5-(5-(4-(hydrazinecarbonyl)benzyl)-1,3,4-oxadiazol-2-yl)pyridin-2-yl)carbamate (150 mg, 0.365 mmol, 1 equiv.) in 5 mL DMF at 0° C. The reaction mixture was let to reach r.t., and then was stirred over 1 h. Conversion was confirmed by LC-MS. The reaction mixture was diluted with EtOAc, washed with sat. aq. NaHCO₃ (4 times) and brine, dried over MgSO₄, evaporated and dried in vacuum. The residue obtained was submitted to prep-HPLC. After evaporation of fractions 15 mg of the desired product were obtained (0.032 mmol, 9% yield).

Step F

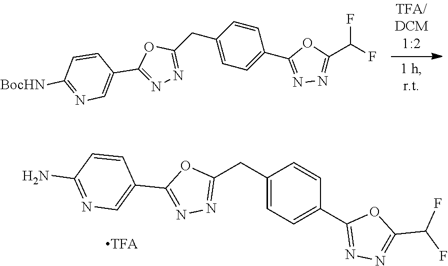 [SMILES: CC(C)(C)OC(=O)Nc1ccc(-c2nnc(Cc3ccc(-c4nnc(C(F)F)o4)cc3)o2)cn1 ; Nc1ccc(-c2nnc(Cc3ccc(-c4nnc(C(F)F)o4)cc3)o2)cn1]

tert-butyl (5-(5-(4-(5-(difluoromethyl)-1,3,4-oxadiazol-2-yl)benzyl)-1,3,4-oxadiazol-2-yl)pyridin-2-yl)carbamate (15 mg, 0.032 mmol, 1 equiv.) was dissolved in a 50% mixture of TFA (10 equiv.) in DCM.

The reaction mixture was stirred at r.t. over 1 h, monitoring conversion by TLC. The mixture was evaporated to dryness, and the residue was triturated with ether to obtain pure product as a TFA salt (15 mg, 0.032, 100% yield, m/z 371.2 [MH+]).

The following compound was synthesized according to the same procedure:

| Compd. | Structure | m/z [MH+] |
|---|---|---|
| 170 | 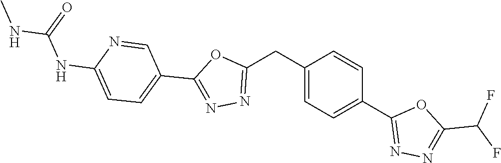 [SMILES: CNC(=O)Nc1ccc(-c2nnc(Cc3ccc(-c4nnc(C(F)F)o4)cc3)o2)cn1] | 428.15 |

### Example 54. Synthesis of 5-(5-(4-(5-(difluoromethyl)-1,3,4-oxadiazol-2-yl)benzyl)isoxazol-3-yl)pyridin-2-amine (Compd. 202)

Step A

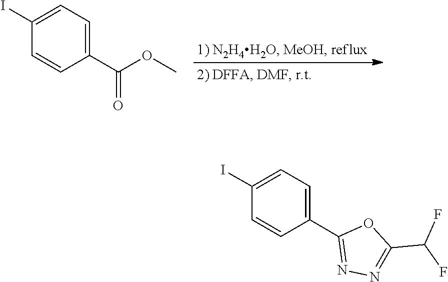

Methyl 4-iodobenzoate (5 g, 19.3 mmol, 1 equiv.) was dissolved in MeOH (5 mL), then hydrazine monohydrate was added (5 equiv.) under stirring. Mixture was stirred at 70° C. overnight. Full conversion of methyl ester to hydrazide was observed by LC-MS (and TLC). The reaction mixture was concentrated under reduced pressure and the residue was diluted in water and extracted with ethyl acetate. The organic phase was washed with sat. aq. NaHCO₃ and brine, dried over Na₂SO₄, filtered and concentrated under reduced pressure. 4.37 g (16.2 mmol) of the intermediate hydrazide were obtained.

The crude intermediate was dissolved in dry DMF (3 mL) under argon. Difluoroacetic anhydride (4 equiv.) was slowly added, keeping temperature below 30° C. (ice/NaCl bath). After addition was complete the temperature was let to reach r.t. The flask was sealed and the reaction mixture was stirred at 70° C. over 3 h. Full conversion was observed by LC-MS, 50% of the desired product formed.

The reaction mixture was diluted with water forming a white precipitate which was collected by filtration, rinsed with water and dried on air overnight. The obtained solid was suspended in 60 mL chloroform, filtered and rinsed twice with more chloroform. The filtrate was concentrated and the residue was dried in vacuo (3.5 g, 9.7 mmol, 50% yield).

Step B

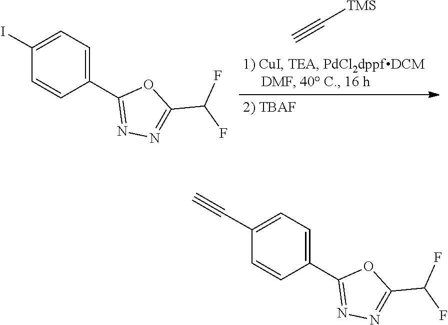

Triethylamine (1 equiv.) and [1,1′-Bis(diphenylphosphino)ferrocene]dichloropalladium(II) dichloromethane complex (0.1 equiv.) were added to a degassed mixture of 2-(difluoromethyl)-5-(4-iodophenyl)-1,3,4-oxadiazole (1.5 g, 4.6 mmol, 1 equiv.), ethynyl(trimethyl)silane (1.5 equiv.) and copper iodide (0.1 equiv.) in 20 mL DMF. The reaction mixture was degassed for 20 min, heated at 40° C. and stirred overnight. Full conversion to the desired intermediate was observed by LC-MS.

Tetrabutylammonium fluoride (1 equiv.) was added to the reaction mixture, which was stirred at r.t. over 1 h. The reaction mixture was diluted with water and extracted with MTBE (3 times). Combined organic layers were washed with sat. aq. NaHCO₃, dried over Na₂SO₄, filtered, concentrated under reduced pressure. The crude residue was purified by flash column chromatography (DCM), to obtain 230 mg (1 mmol, 22% yield) of the desired product.

Step C

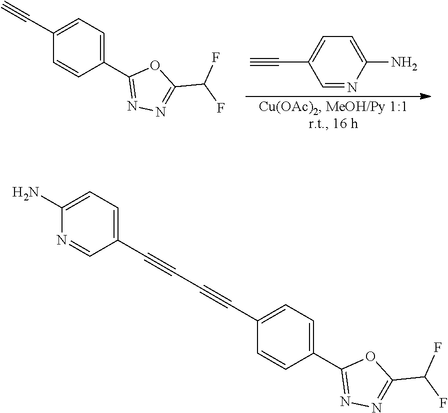

2-(difluoromethyl)-5-(4-ethynylphenyl)-1,3,4-oxadiazole (210 mg, 0.95 mmol, 1 equiv.) and 5-ethynylpyridin-2-amine (5 equiv.) were dissolved in a 1:1 mixture of methanol and pyridine (10 mL). The mixture was degassed with argon, and copper acetate (2 equiv.) was added under a stream of argon. The reaction mixture was stirred at r.t. overnight.

The reaction mixture was then filtered, and the obtained solid was washed with MeOH, EtOAc and DCM. Combined organic phases were concentrated. The residue was dissolved in EtOAc and washed with water (3 times), dried over MgSO₄, filtered and evaporated. Crude product was purified by flash column chromatography (EtOAc/DCM) obtaining 50 mg of the desired product (0.15 mmol, 15% yield).

Step D

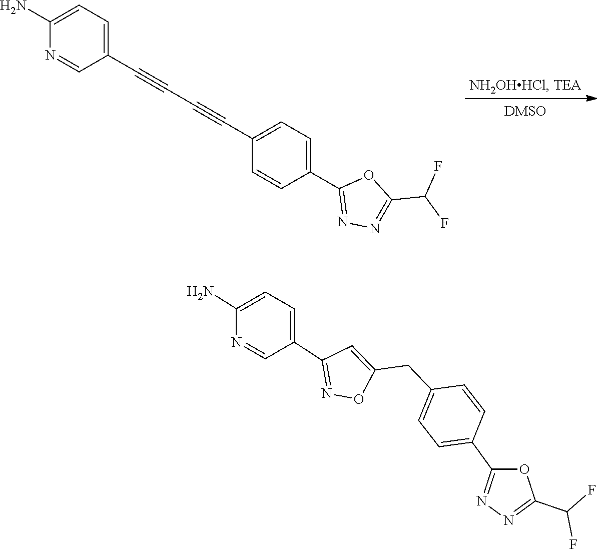 [SMILES: Nc1ccc(C#CC#Cc2ccc(-c3nnc(C(F)F)o3)cc2)cn1 ; Nc1ccc(-c2cc(Cc3ccc(-c4nnc(C(F)F)o4)cc3)on2)cn1]

5-[4-[4-[5-(difluoromethyl)-1,3,4-oxadiazol-2-yl]phenyl]buta-1,3-diynyl]pyridin-2-amine (50 mg, 0.15 mmol, 1 equiv.) was dissolved in DMSO (2 mL). Triethylamine (6 equiv.) and hydroxylamine hydrochloride (3.5 equiv.) were added. The reaction mixture was stirred at 110° C. overnight. After cooling to r.t. the mixture was submitted to prep-HPLC (0.1% FA/ACN/water), affording the desired product (4.4 mg, 0,012 mmol, 9.6% yield, m/z 369.71 [MH+]).

The following compound was prepared according to the same procedure:

| Compd. | Structure | m/z [MH+] |
|---|---|---|
| 366 | 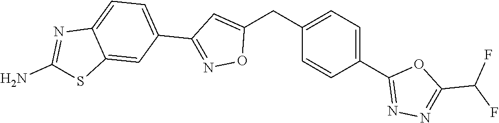 [SMILES: Nc1nc2ccc(-c3cc(Cc4ccc(-c5nnc(C(F)F)o5)cc4)on3)cc2s1] | 426.10 |

### Example 55. Synthesis 2-(difluoromethyl)-5-(4-((5-phenyl-1,3,4-thiadiazol-2-yl)methyl)phenyl)-1,3,4-oxadiazole (Compd. 238)

Step A

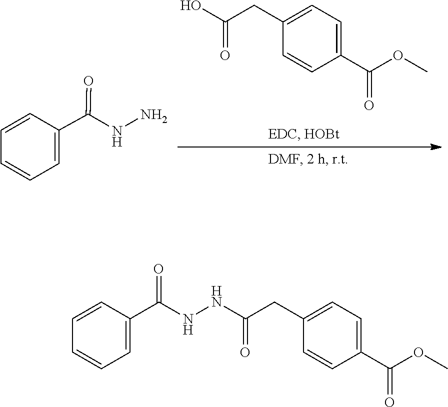 [SMILES: NNC(=O)c1ccccc1 ; COC(=O)c1ccc(CC(=O)O)cc1 ; COC(=O)c1ccc(CC(=O)NNC(=O)c2ccccc2)cc1]

A solution of 2-(4-(methoxycarbonyl)phenyl)acetic acid (300 mg, 1.5 mmol, 1 equiv.), EDC (1.2 equiv.) and HOBt (1.1 equiv.) in 4 mL DMF was stirred at r.t. over 10 minutes. Benzohydrazide (1 equiv.) was added, and the reaction mixture was stirred for 2 h. Full conversion to product was detected by LC-MS. The reaction mixture was diluted with EtOAc, washed with sat. aq. NaHCO₃ and brine, dried and evaporated in vacuum to get pure target compound (343 mg, 1.1 mmol, 71% yield).

Step B

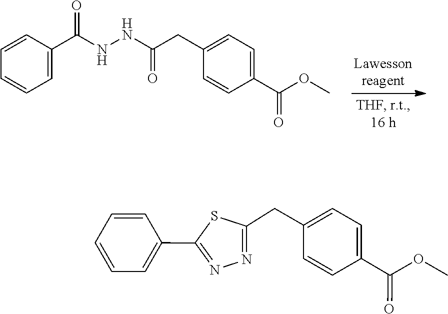 [SMILES: COC(=O)c1ccc(CC(=O)NNC(=O)c2ccccc2)cc1 ; COC(=O)c1ccc(Cc2nnc(-c3ccccc3)s2)cc1]

A mixture of methyl 4-(2-(2-benzoylhydrazineyl)-2-oxoethyl)benzoate (343 mg, 1.1 mmol, 1 equiv.) and Lawesson's reagent (1.5 equiv.) in THF (5 mL) was stirred at r.t. overnight. The reaction mixture was diluted with EtOAc, washed with sat. aq. NaHCO₃, water and brine, dried over MgSO₄ and evaporated in vacuum. The target compound thus obtained was used in the next step without further purification (340 mg, 1.1 mmol, 99% yield).

Step C

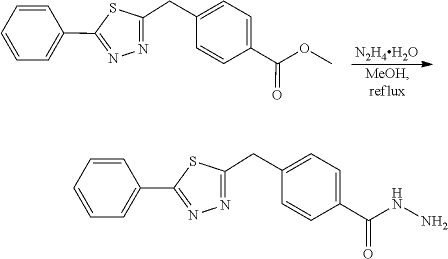

A solution of methyl 4-((5-phenyl-1,3,4-thiadiazol-2-yl)methyl)benzoate (340 mg, 1.1 mmol, 1 equiv.) and hydrazine hydrate (5 equiv.) in 5 mL methanol was stirred at reflux overnight. Full conversion was detected by LC-MS. The reaction mixture was concentrated. The residue was suspended in acetonitrile and evaporated twice to afford the desired product, which was dried under vacuum (340 mg, 1.1 mmol, 100% yield).

Step D

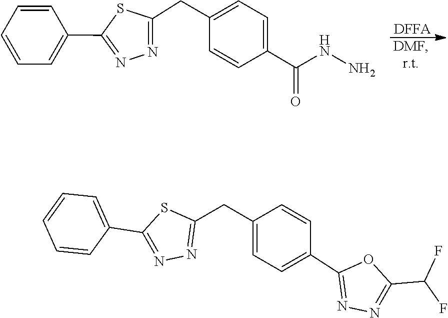 [SMILES: NNC(=O)c1ccc(Cc2nnc(-c3ccccc3)s2)cc1 ; "DFFA" ; "DMF, r.t." ; FC(F)c1nnc(-c2ccc(Cc3nnc(-c4ccccc4)s3)cc2)o1]

Difluoroacetic anhydride (3 equiv.) was added to a solution of 3-(5-(4-(hydrazinecarbonyl)benzyl)-1,2,4-oxadiazol-3-yl)benzamide (340 mg, 1.1 mmol, 1 equiv.) in 5 mL DMF at 0° C. The reaction mixture was heated to 70° C. and stirred over 6 h. Full conversion was observed by LC-MS. The reaction mixture was diluted with EtOAc, washed with sat. aq. NaHCO₃, water and brine, dried over MgSO₄, filtered and concentrated in vacuum. The residue was purified by prep-HPLC to give target compound (41 mg, 0.11 mmol, 10% yield, m/z 370.85 [MH+]).

### Example 56. Synthesis N-(5-(5-((5-(5-(difluoromethyl)-1,3,4-oxadiazol-2-yl)pyridin-2-yl)methyl)-1,3,4-thiadiazol-2-yl)pyridin-2-yl)-2,2-difluoroacetamide (Compd. 102)

Step A

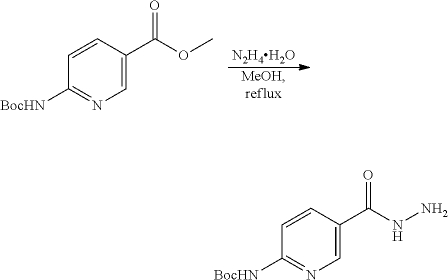

A solution of methyl 6-((tert-butoxycarbonyl)amino)nicotinate (1 g, 3.9 mmol, 1 equiv.) and hydrazine hydrate (5 equiv.) in 20 mL MeOH was stirred at 70° C. overnight. Full conversion was detected by TLC (DCM/MeOH 95:5). The reaction mixture was concentrated to dryness. The residue was resuspended in acetonitrile and evaporated again to yield pure target compound (1 g, 3.9 mmol, 100% yield).

Step B

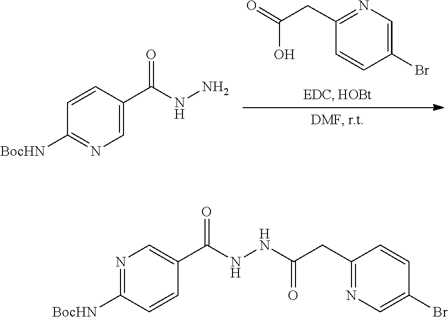 [SMILES: CC(C)(C)OC(=O)Nc1ccc(C(=O)NN)cn1 ; O=C(O)Cc1ccc(Br)cn1 ; CC(C)(C)OC(=O)Nc1ccc(C(=O)NNC(=O)Cc2ccc(Br)cn2)cn1]

A solution of 2-(5-bromopyridin-2-yl)acetic acid (342 mg, 1.58 mmol, 1 equiv.), EDC (1.2 equiv.) and HOBt (1.1 equiv.) in 4 mL DMF was stirred at r.t. over 15 minutes. tert-butyl (5-(hydrazinecarbonyl)pyridin-2-yl)carbamate (1 equiv.) was added, and the reaction mixture was stirred for 3 h. Full conversion to product was detected by LC-MS. The reaction mixture was diluted with water. The precipitate which formed was collected by filtration and rinsed with water (5 times), dried under vacuum to give pure target product as yellow solid (483 mg, 1.07 mmol, 68% yield).

Step C

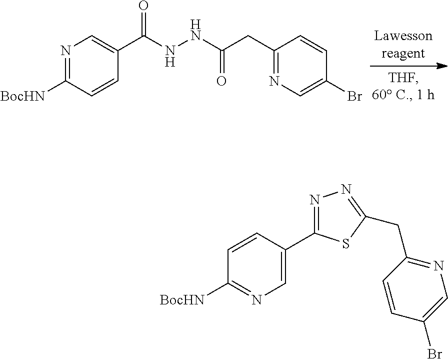 [SMILES: CC(C)(C)OC(=O)Nc1ccc(C(=O)NNC(=O)Cc2ccc(Br)cn2)cn1 ; CC(C)(C)OC(=O)Nc1ccc(-c2nnc(Cc3ccc(Br)cn3)s2)cn1]

A mixture of tert-butyl (5-(2-(2-(5-bromopyridin-2-yl)acetyl)hydrazine-1-carbonyl)pyridin-2-yl)carbamate (483 mg, 1.07 mmol, 1 equiv.) and Lawesson's reagent (1.5 equiv.) in THF (5 mL) was stirred at 60° C. over 1 h. The reaction mixture was diluted with EtOAc, washed with sat. aq. NaHCO₃, water and brine, dried over MgSO₄ and evaporated in vacuum. The residue was purified by flash chromatography (hexane/EtOAc 9:1 to 1:1) to give target compound as pure solid (221 mg, 0.49 mmol, 46% yield).

Step D

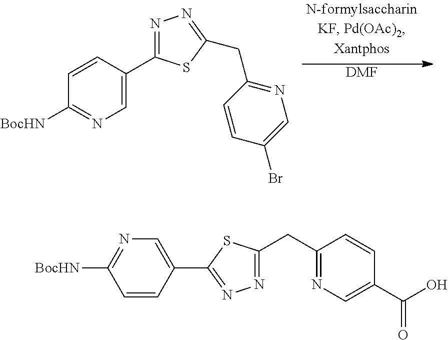

A flame-dried flask was charged with tert-butyl (5-(5-((5-bromopyridin-2-yl)methyl)-1,3,4-thiadiazol-2-yl)pyridin-2-yl)carbamate (220 mg, 0.49 mmol, 1 equiv.), N-formylsaccharin (1.5 equiv.), potassium fluoride (2.5 equiv.) and Xantphos (0.1 equiv.). Dry DMF (1 mL) was added. Pd(OAc)₂ (0.05 equiv.) was added to the resulting mixture, which was degassed with Ar and heated at 80° C. under stirring over 2 days. Partial conversion of the starting material was detected by LC-MS. The reaction mixture was diluted with EtOAc, washed with sat. aq. NaHCO₃ (4 times) and brine, dried over MgSO₄, filtered and evaporated. The residue obtained was purified by column chromatography (DCM/MeOH/formic acid 9:1:0 to 8:2:0 to 9:1:0.02) to give target compound (59 mg, 0.14 mmol, 29% yield).

Step E

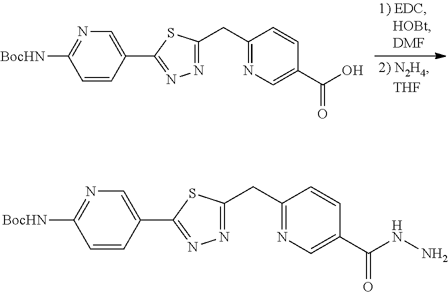 [SMILES: CC(C)(C)OC(=O)Nc1ccc(-c2nnc(Cc3ccc(C(=O)O)cn3)s2)cn1 ; CC(C)(C)OC(=O)Nc1ccc(-c2nnc(Cc3ccc(C(=O)NN)cn3)s2)cn1]

A solution of 64 (5-(6-((tert-butoxycarbonyl)amino)pyridin-3-yl)-1,3,4-thiadiazol-2-yl)methyl)nicotinic acid (59 mg, 0.14 mmol, 1 equiv.), EDC (1.2 equiv) and HOBt (1.2 equiv) in 2 mL DMF was stirred at r.t. for 10 min. 1M hydrazine solution in THF (4 equiv.) was added and the reaction mixture was stirred for 5 h. Partial conversion was detected by LC-MS. The mixture was evaporated to dryness and purified by flash column chromatography (DCM/MeOH 95:5 to 9:1) to give target compound (7 mg, 0.016 mmol, 11% yield).

Step F

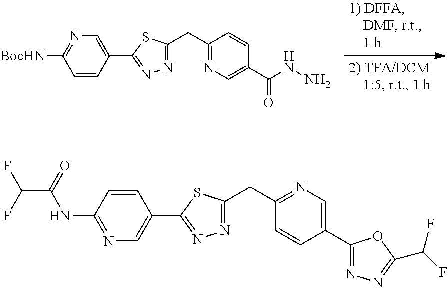 [SMILES: CC(C)(C)OC(=O)Nc1ccc(-c2nnc(Cc3ccc(C(=O)NN)cn3)s2)cn1 ; O=C(Nc1ccc(-c2nnc(Cc3ccc(-c4nnc(C(F)F)o4)cn3)s2)cn1)C(F)F]

Difluoroacetic anhydride (4 equiv.) was added to a solution of tert-butyl (5-(5-((5-(hydrazinecarbonyl)pyridin-2-yl)methyl)-1,3,4-thiadiazol-2-yl)pyridin-2-yl)carbamate (7 mg, 0.016 mmol, 1 equiv.) in 0.5 mL DMF. The reaction mixture was stirred at r.t. over 1 h. According to LC-MS, the starting material was fully converted to Boc-protected intermediate and desired product. The reaction mixture was diluted with EtOAc, washed with sat. aq. NaHCO₃, water and brine, dried over MgSO₄, filtered and concentrated in vacuum.

The crude intermediate was suspended in 1:5 TFA:DCM mixture (600 μL), and the resulting solution was stirred at r.t. over 2 h. Full conversion to product was observed by LC-MS. The reaction mixture was diluted with EtOAc, washed with sat. aq. NaHCO₃ (2 times) and brine, dried over MgSO₄, filtered, evaporated and purified by prep-HPLC to give pure target compound (0.6 mg, 0.001 mmol, 9% yield, m/z 465.65 [MH+]).

### Example 57. Synthesis of 6-(1-((5-(5-(difluoromethyl)-1,3,4-oxadiazol-2-yl)pyridin-2-yl)methyl)-1H-imidazol-4-yl)isoindolin-1-one (Compd. 292)

Step A

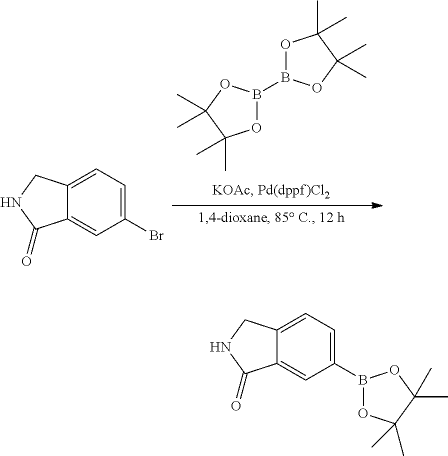

A solution of 6-bromo-2,3-dihydroisoindol-1-one (500 mg, 2.36 mmol, 1 equiv.), bis(pinacolato)diboron (1.5 equiv.) and potassium acetate (3 equiv.) in 1,4-dioxane (10.0 mL) was degassed by flushing with argon for 15 min. [1,1′-Bis(diphenylphosphino)ferrocene]dichloropalladium(II) (0.1 equiv.) was then added to the reaction mixture, which was degassed again with argon for 15 min. The resulting reaction mixture was heated to 85° C. for 12 h. After confirming the reaction completion by TLC, the reaction mixture was filtered through a Celite® pad. The filtrate was concentrated, and the crude residue thus obtained was suspended in EtOAc and washed with water. The organic layer was dried over Na₂SO₄, filtered and concentrated under reduced pressure. The residue was purified by flash column chromatography (DCM/MeOH 95:5) to give the product as a beige solid (690 mg, 1.87 mmol, 79% yield).

Step B

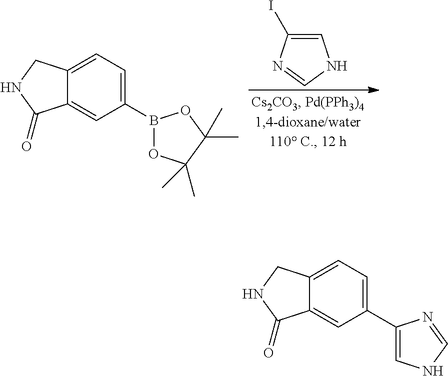 [SMILES: CC1(C)OB(c2ccc3c(c2)C(=O)NC3)OC1(C)C ; Ic1c[nH]cn1 ; O=C1NCc2ccc(-c3c[nH]cn3)cc21]

6-(4,4,5,5-tetramethyl-1,3,2-dioxaborolan-2-yl)isoindolin-1-one (234 mg, 0.9 mmol, 1 equiv.), 4-iodo-1H-imidazole (1 equiv) and cesium carbonate (1.5 equiv.) were dissolved in a 4:1 mixture 1,4-dioxane/water (2.5 mL). Reaction mixture was purged with argon and Tetrakis(triphenylphosphine)palladium(0) (0.05 equiv.) was added. The reaction mixture was stirred at 110° C. for 12 h.

The reaction was then poured into water and extracted with EtOAc. The aqueous phase was further extracted with CHCl₃/IPA 3:1 mixture. The combined organic extracts were dried over Na₂SO₄ and concentrated under reduced pressure. The residue was purified by flash column chromatography (DCM/MeOH 8:2) to give the desired product (60 mg, 0.27 mmol, 30% yield).

Step C

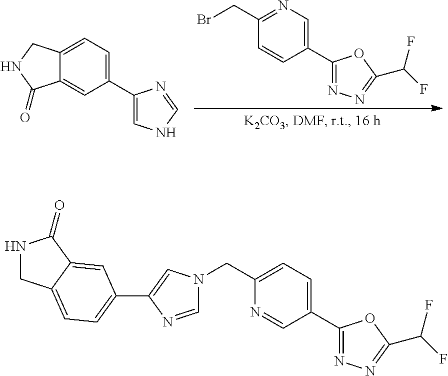 [SMILES: O=C1NCc2ccc(-c3c[nH]cn3)cc21 ; FC(F)c1nnc(-c2ccc(CBr)nc2)o1 ; O=C1NCc2ccc(-c3cn(Cc4ccc(-c5nnc(C(F)F)o5)cn4)cn3)cc21]

2-[6-(bromomethyl)pyridin-3-yl]-5-(difluoromethyl)-1,3,4-oxadiazole (Intermediate A, 39 mg, 0.13 mmol, 1 equiv.) was added to a solution of 6-(1H-imidazol-4-yl)-2,3-dihydroisoindol-1-one (1 equiv.) and potassium carbonate (2 equiv.) in 1 mL DMF. The flask was sealed and the reaction mixture was stirred at r.t. overnight. After determining full conversion of the starting material, the reaction mixture was diluted with water and extracted with EtOAc. Organic layers were dried over Na₂SO₄ and concentrated under reduced pressure. The residue was purified by prep-HPLC, to obtain pure title compound as a formate salt (12 mg, 0.03 mmol, 21% yield, m/z 409.07 [MH+]).

The following compounds were synthesized according to the same procedure:

| Compd. | Structure | m/z [MH+] |
|---|---|---|
| 294 | 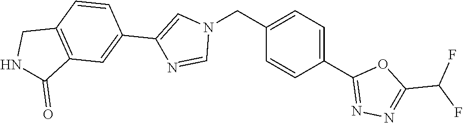 [SMILES: O=C1NCc2ccc(-c3cn(Cc4ccc(-c5nnc(C(F)F)o5)cc4)cn3)cc21] | 408.28 |
| 300 | 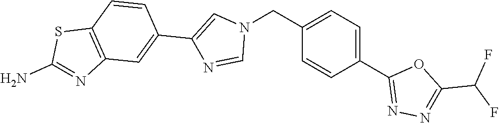 [SMILES: Nc1nc2cc(-c3cn(Cc4ccc(-c5nnc(C(F)F)o5)cc4)cn3)ccc2s1] | 425 |

The following compounds were synthesized according to the same procedure, starting from the corresponding boronate esters (step B):

| Compd. | Structure | m/z [MH+] |
|---|---|---|
| 151 | 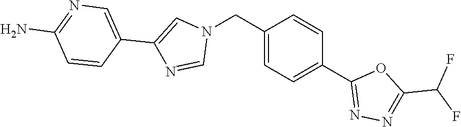 [SMILES: Nc1ccc(-c2cn(Cc3ccc(-c4nnc(C(F)F)o4)cc3)cn2)cn1] | 369.05 |
| 223 | 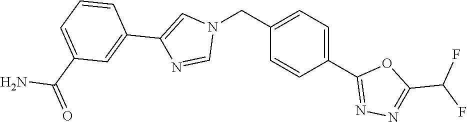 [SMILES: NC(=O)c1cccc(-c2cn(Cc3ccc(-c4nnc(C(F)F)o4)cc3)cn2)c1] | 396.01 |

### Example 58. Synthesis of 6-(1-((5-(5-(difluoromethyl)-1,3,4-oxadiazol-2-yl)pyridin-2-yl)methyl)-1H-imidazol-4-yl)isoindolin-1-one (Compd. 264)

Step A

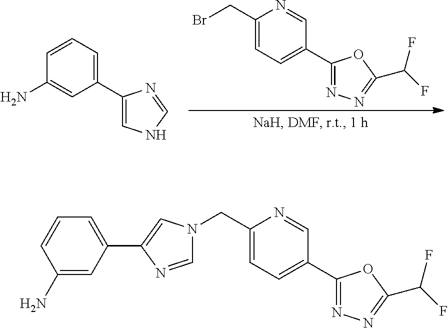 [SMILES: Nc1cccc(-c2c[nH]cn2)c1 ; FC(F)c1nnc(-c2ccc(CBr)nc2)o1 ; Nc1cccc(-c2cn(Cc3ccc(-c4nnc(C(F)F)o4)cn3)cn2)c1]

3-(1H-imidazol-4-yl)aniline (1.25 equiv.) was dissolved in 3 mL DMF, and sodium hydride (1.25 equiv.) was added. After stirring the mixture over 30 min 2-[6-(bromomethyl)pyridin-3-yl]-5-(difluoromethyl)-1,3,4-oxadiazole (Intermediate A, 146 mg, 0.5 mmol, 1 equiv.) was added. The reaction mixture was stirred for 1 h, and then it was diluted with water and extracted with EtOAc. Organic layers were dried over Na₂SO₄, filtered, concentrated. Crude residue was used in the next step without any further purification (165 mg, 0.28 mmol, 45% yield).

Step B

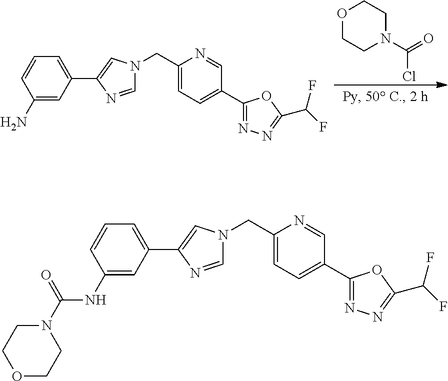 [SMILES: Nc1cccc(-c2cn(Cc3ccc(-c4nnc(C(F)F)o4)cn3)cn2)c1 ; O=C(Cl)N1CCOCC1 ; O=C(Nc1cccc(-c2cn(Cc3ccc(-c4nnc(C(F)F)o4)cn3)cn2)c1)N1CCOCC1]

3-[1-[[5-[5-(difluoromethyl)-1,3,4-oxadiazol-2-yl]pyridin-2-yl]methyl]imidazol-4-yl]aniline (135 mg, 0.23 mmol, 1 equiv.) was dissolved in 5 mL pyridine, and morpholine-4-carbonyl chloride (2.5 equiv.) was added. The reaction mixture was stirred at 50° C. over 2 h. Upon completion, the mixture was diluted with water and extracted with EtOAc. Organic phases were dried over Na₂SO₄, filtered and concentrated. The crude residue was purified by prep-HPLC (ACN/water) to obtain the desired product (45 mg, 0.09 mmol, 38% yield, m/z 481.86 [MH+]).

The following compound was synthesized according to the same procedure:

| Compd. | Structure | m/z [MH+] |
|---|---|---|
| 267 | 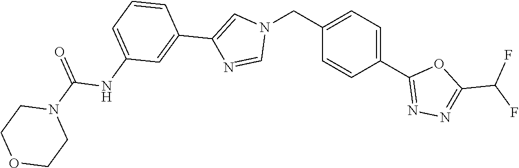 [SMILES: O=C(Nc1cccc(-c2cn(Cc3ccc(-c4nnc(C(F)F)o4)cc3)cn2)c1)N1CCOCC1] | 481.21 |

The following compound was synthesized according to step A of this procedure:

| Compd. | Structure | m/z [MH+] |
|---|---|---|
| 197 | 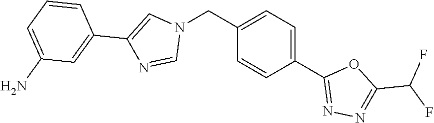 [SMILES: Nc1cccc(-c2cn(Cc3ccc(-c4nnc(C(F)F)o4)cc3)cn2)c1] | 368.03 |

### Example 59. Synthesis of 6-(1-((5-(5-(difluoromethyl)-1,3,4-oxadiazol-2-yl)pyridin-2-yl)methyl)-1H-imidazol-4-yl)isoindolin-1-one (Compd. 22)

Step A

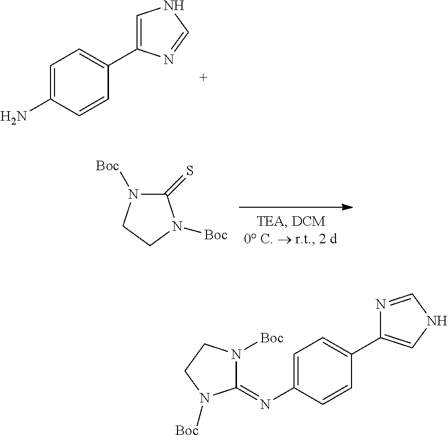 [SMILES: Nc1ccc(-c2c[nH]cn2)cc1 ; CC(C)(C)OC(=O)N1CCN(C(=O)OC(C)(C)C)C1=S ; CC(C)(C)OC(=O)N1CCN(C(=O)OC(C)(C)C)C1=Nc1ccc(-c2c[nH]cn2)cc1]

Mercury(II) chloride (1.1 equiv.) was added to a solution of 4-(1H-imidazol-4-yl)aniline (250 mg, 1.57 mmol, 1 equiv.), di-tert-butyl 2-thioxoimidazolidine-1,3-dicarboxylate (1 equiv.) and triethylamine (3.1 equiv.) in 10 mL DCM at 0° C. The resulting mixture was stirred at 0° C. for 1 h and then at r.t. for 2 days. The reaction mixture was diluted with water and DCM. The mixture was filtered and the filtrate was washed with sat. aq. NaHCO₃, brine, dried over MgSO₄, filtered, and concentrated under reduced pressure. The resulting beige solid was used in the next step without any further purification (470 mg, 1.1 mmol, 70% yield).

Step B

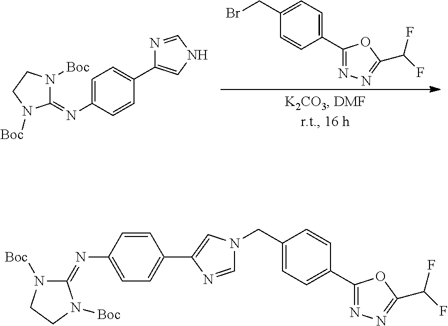 [SMILES: CC(C)(C)OC(=O)N1CCN(C(=O)OC(C)(C)C)C1=Nc1ccc(-c2c[nH]cn2)cc1 ; FC(F)c1nnc(-c2ccc(CBr)cc2)o1 ; CC(C)(C)OC(=O)N1CCN(C(=O)OC(C)(C)C)C1=Nc1ccc(-c2cn(Cc3ccc(-c4nnc(C(F)F)o4)cc3)cn2)cc1]

di-tert-butyl 2-((4-(1H-imidazol-4-yl)phenyl)imino)imidazolidine-1,3-dicarboxylate (250 mg, 0.58 mmol, 1 equiv.) and potassium carbonate (1.1 equiv.) were suspended in 2.5 mL DMF. After 15 min 2-[4-(bromomethyl)phenyl]-5-(difluoromethyl)-1,3,4-oxadiazole (Intermediate B, 1 equiv.) was added to the suspension and the reaction mixture was stirred at r.t. overnight. Water was added to the reaction mixture, which was extracted with EtOAc. The organic phase was washed with sat. aq. NaHCO₃ (3×) and brine, dried over MgSO₄, filtered, concentrated under reduced pressure. The residue was purified by flash column chromatography (hexane/EtOAc 3:7 to 5:95) affording the product as a purple solid (150 mg, 0.23 mmol, 40% yield).

Step C

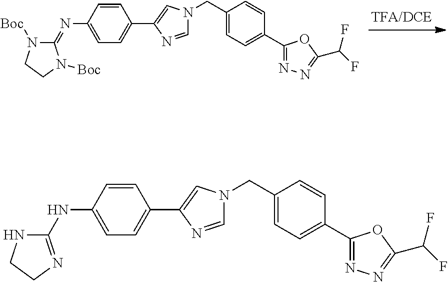 [SMILES: CC(C)(C)OC(=O)N1CCN(C(=O)OC(C)(C)C)C1=Nc1ccc(-c2cn(Cc3ccc(-c4nnc(C(F)F)o4)cc3)cn2)cc1 ; FC(F)c1nnc(-c2ccc(Cn3cnc(-c4ccc(NC5=NCCN5)cc4)c3)cc2)o1]

di-tert-butyl 2-((4-(1-(4-(5-(difluoromethyl)-1,3,4-oxadiazol-2-yl)benzyl)-1H-imidazol-4-yl)phenyl)imino)imidazolidine-1,3-dicarboxylate (150 mg, 0.24 mmol, 1 equiv.) was dissolved in DCE and TFA (15 equiv.) was added. The reaction mixture was stirred at r.t. overnight, and then concentrated under reduced pressure. The residue was dissolved in ethyl acetate and washed with sat. aq. NaHCO₃ and brine, dried over MgSO₄, filtered and concentrated under reduced pressure. The residue was purified by prep-HPLC (ACN/water/FA) and lyophilized to afford the product as a white solid (70 mg, 0.16 mmol, 68% yield, m/z 436.07 [MH+]).

The following compounds were synthesized according to the same procedure:

| Compd. | Structure | m/z [MH+] |
|---|---|---|
| 20 | 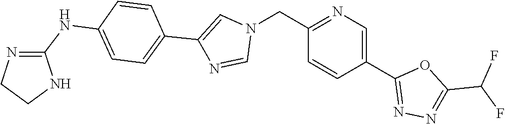 [SMILES: FC(F)c1nnc(-c2ccc(Cn3cnc(-c4ccc(NC5=NCCN5)cc4)c3)nc2)o1] | 437.08 |
| 357 | 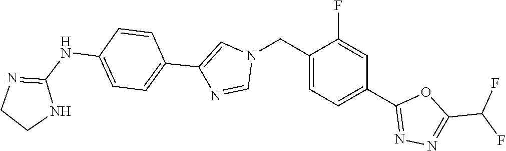 [SMILES: Fc1cc(-c2nnc(C(F)F)o2)ccc1Cn1cnc(-c2ccc(NC3=NCCN3)cc2)c1] | 454.30 |
| 358 | 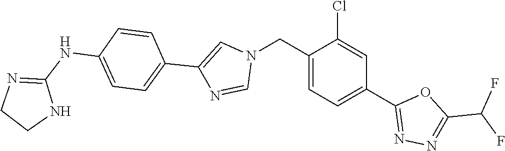 [SMILES: FC(F)c1nnc(-c2ccc(Cn3cnc(-c4ccc(NC5=NCCN5)cc4)c3)c(Cl)c2)o1] | 470.23 |
| 359 | 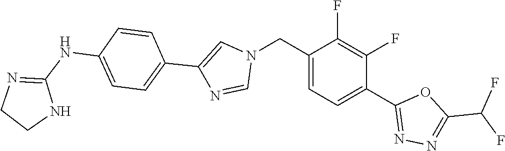 [SMILES: Fc1c(Cn2cnc(-c3ccc(NC4=NCCN4)cc3)c2)ccc(-c2nnc(C(F)F)o2)c1F] | 472.4 |
| 360 | 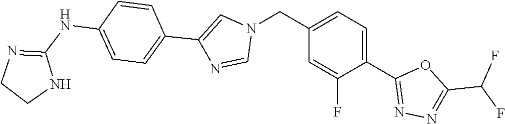 [SMILES: Fc1cc(Cn2cnc(-c3ccc(NC4=NCCN4)cc3)c2)ccc1-c1nnc(C(F)F)o1] | 454.15 |
| 361 | 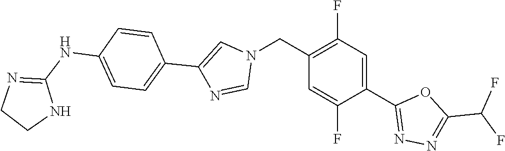 [SMILES: Fc1cc(-c2nnc(C(F)F)o2)c(F)cc1Cn1cnc(-c2ccc(NC3=NCCN3)cc2)c1] | 472.17 |
| 368 | 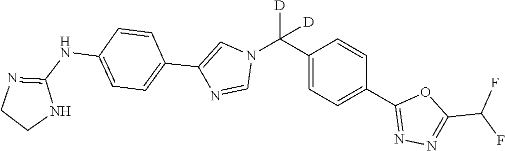 [SMILES: [2H]C([2H])(c1ccc(-c2nnc(C(F)F)o2)cc1)n1cnc(-c2ccc(NC3=NCCN3)cc2)c1] | 437.97 |

### Example 60. Synthesis of 5-(1-((5-(5-(difluoromethyl)-1,3,4-oxadiazol-2-yl)pyridin-2-yl)methyl)-1H-imidazol-4-yl)pyridin-2-amine (Compd. 12) and 5-(1-((5-(5-(difluoromethyl)-1,3,4-oxadiazol-2-yl)pyridin-2-yl)methyl)-1H-imidazol-4-yl)pyridin-2-amine (Compd. 126)

Step A

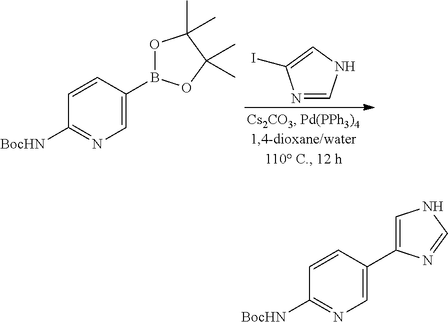

tert-Butyl (5-(4,4,5,5-tetramethyl-1,3,2-dioxaborolan-2-yl)pyridin-2-yl)carbamate (400 mg, 1.25 mmol, 1 equiv.), 4-iodo-1H-imidazole (1 equiv.), cesium carbonate (2.5 equiv.) and tetrakis(triphenylphosphine)palladium(0) (0.1 equiv.) were suspended in a 3:1 dioxane/water solution (12 mL) and degassed with Ar. The reaction mixture was stirred at 85° C. overnight. Conversion was confirmed by LC-MS.

The reaction mixture was diluted with EtOAc and filtered through a pad of Celite®. The organic phase was washed with water and evaporated. Crude was purified by flash column chromatography to obtain 228 mg of the desired product (0.876 mmol, 70% yield).

Step B

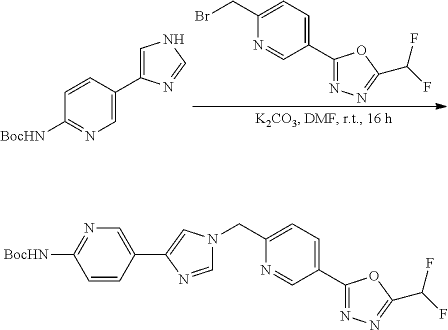 [SMILES: CC(C)(C)OC(=O)Nc1ccc(-c2c[nH]cn2)cn1 ; FC(F)c1nnc(-c2ccc(CBr)nc2)o1 ; CC(C)(C)OC(=O)Nc1ccc(-c2cn(Cc3ccc(-c4nnc(C(F)F)o4)cn3)cn2)cn1]

A mixture of tert-butyl (5-(1H-imidazol-4-yl)pyridin-2-yl)carbamate (1.25 equiv.) and potassium carbonate (2.5 equiv.) in 5 mL DMF was stirred at r.t. for 30 min. 2-[6-(bromomethyl)pyridin-3-yl]-5-(difluoromethyl)-1,3,4-oxadiazole (Intermediate A, 203 mg, 0.7 mmol, 1 equiv.) was added and the reaction mixture was stirred overnight. The reaction mixture was diluted with EtOAc, washed with sat. aq. NaHCO₃ and brine, dried over Na₂SO₄, filtered and concentrated. The crude residue was purified by flash column chromatography (DCM/MeOH 98:2 to 9:1) to get target compound (50 mg, 0.1 mmol, 15% yield).

Step C

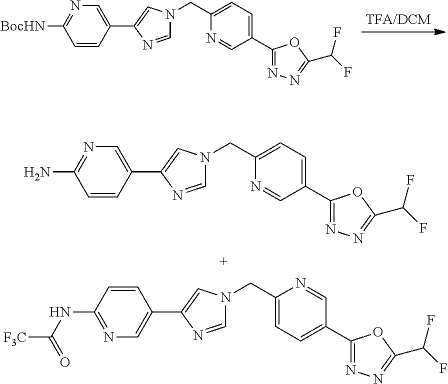 [SMILES: CC(C)(C)OC(=O)Nc1ccc(-c2cn(Cc3ccc(-c4nnc(C(F)F)o4)cn3)cn2)cn1 ; Nc1ccc(-c2cn(Cc3ccc(-c4nnc(C(F)F)o4)cn3)cn2)cn1 ; O=C(Nc1ccc(-c2cn(Cc3ccc(-c4nnc(C(F)F)o4)cn3)cn2)cn1)C(F)(F)F]

tert-Butyl (5-(1-((5-(5-(difluoromethyl)-1,3,4-oxadiazol-2-yl)pyridin-2-yl)methyl)-1H-imidazol-4-yl)pyridin-2-yl)carbamate (70 mg, 0.15 mmol, 1 equiv.) was dissolved in 0.5 mL DCM, and TFA (10 equiv.) was added at r.t. According to LC-MS conversion was complete after 3 h. The reaction mixture was diluted with EtOAc, washed with sat. aq. NaHCO₃ (2×) and brine. Organic layer was dried over Na₂SO₄, filtered and evaporated under vacuum. The residue obtained was purified by prep-HPLC to give pure separated target compounds:

compd. 12: 18 mg, 0.05 mmol, 32% yield (m/z 369.73 [MH+])

compd. 126: 5 mg, 0.01 mmol, 7% yield (m/z 447.89 [MH+])

The following compound was synthesized following the same procedure:

| Compd. | Structure | m/z [MH+] |
|---|---|---|
| 305 | 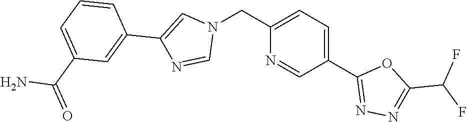 [SMILES: NC(=O)c1cccc(-c2cn(Cc3ccc(-c4nnc(C(F)F)o4)cn3)cn2)c1] | 397.05 |

### Example 61. Synthesis of 5-(1-(4-(5-(difluoromethyl)-1,3,4-oxadiazol-2-yl)benzyl)-1H-pyrazol-4-yl)pyridin-2-amine (Compd. 211)

Step A

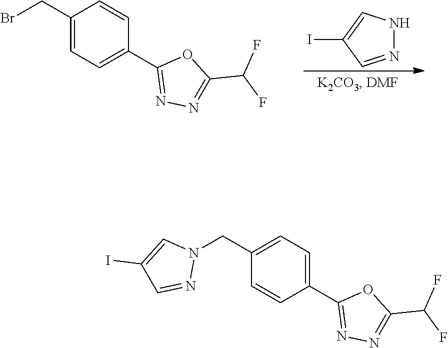 [SMILES: FC(F)c1nnc(-c2ccc(CBr)cc2)o1 ; Ic1cn[nH]c1 ; FC(F)c1nnc(-c2ccc(Cn3cc(I)cn3)cc2)o1]

2-[4-(bromomethyl)phenyl]-5-(difluoromethyl)-1,3,4-oxadiazole (Intermediate B, 500 mg, 1.73 mmol, 1.0 equiv.) and 4-iodo-1H-pyrazole (1 equiv.) were dissolved in DMF (10 mL). Potassium carbonate was then added (2.0 equiv.), and the mixture was stirred at r.t. overnight. The mixture was diluted with EtOAc and washed with water, sat. aq. NaHCO₃ and brine, dried over Na₂SO₄, filtered and concentrated in vacuo.

The residue was purified by flash column chromatography (hexane/EtOAc 4:1) to obtain the desired product (680 mg, 1.69 mmol, 98% yield).

Step B

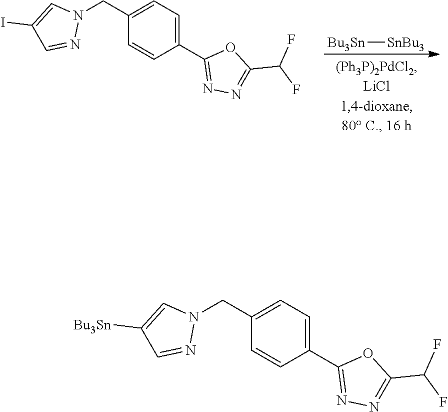

A solution of 2-(difluoromethyl)-5-(4-((4-iodo-1H-pyrazol-1-yl)methyl)phenyl)-1,3,4-oxadiazole (1079 mg, 2.68 mmol, 1 equiv.), LiCl (6 equiv.) and bis(triphenylphosphine)palladium (II) chloride (0.05 equiv.) in 10 mL 1,4-dioxane was degassed with argon. Bis(tributyltin) was added (1.1 equiv.), the flask was sealed and the reaction mixture was stirred at 80° C. overnight. The mixture was let to reach r.t., and then volatiles were removed under vacuum. The residue was partitioned between EtOAc and water. The organic layer was dried, filtered and concentrated under reduced pressure. The residue was purified by flash column chromatography (hexane/EtOAc 4:1) to obtain 175 mg of the desired product (0.23 mmol, 8% yield).

Step C

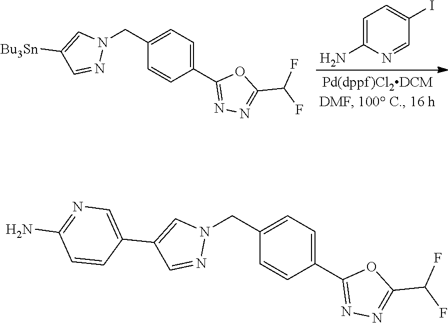

A solution of 2-(difluoromethyl)-5-(4-((4-(tributylstannyl)-1H-pyrazol-1-yl)methyl)phenyl)-1,3,4-oxadiazole (175 mg, 0.23 mmol, 1 equiv.) and 5-iodopyridin-2-amine (1 equiv.) in 2 mL DMF was degassed with argon. [1,1′-bis(diphenylphosphino)ferrocene]dichloropalladium(II) DCM complex (0.05 equiv.) was added, the flask was sealed and the reaction mixture was stirred at 100° C. overnight. Subsequently, the mixture was cooled to r.t., filtered through a Celite® pad and concentrated under reduced pressure. The residue was purified by flash column chromatography (DCM/MeOH 9:1) to give product, which was additionally triturated with pentane and dried in vacuo (53 mg, 0.14 mmol, 61% yield, m/z 369.06 [MH+]).

### Example 62. Synthesis of 5-(1-((5-(5-(difluoromethyl)-1,3,4-oxadiazol-2-yl)pyridin-2-yl)methyl)-1H-pyrazol-4-yl)pyridin-2-amine (Compd. 13)

Step A

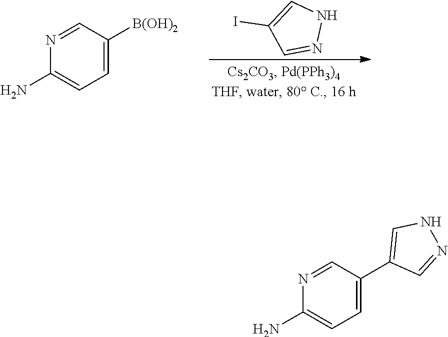 [SMILES: Nc1ccc(B(O)O)cn1 ; Ic1cn[nH]c1 ; Nc1ccc(-c2cn[nH]c2)cn1]

A mixture of (6-aminopyridin-3-yl)boronic acid (250 mg, 1.14 mmol, 1 equiv.), 4-iodo-1H-pyrazole (1 equiv.) and cesium carbonate (3 equiv.) in 3 mL water/THF 2:1 was degassed with argon. Tetrakis(triphenylphosphine)palladium (0.05 equiv.) was added, and the resulting mixture was degassed again. The reaction vessel was sealed, and the mixture was stirred under inert atmosphere at 80° C. overnight. Full conversion to product was confirmed by LC-MS. The mixture was diluted with EtOAc and water. Phases were separated and the organic layer was further extracted with water (twice). The combined aqueous layers were washed with EtOAc, concentrated, reevaporated from MeCN (3 times) and dried in vacuum. The residue obtained (mixture with cesium carbonate) was used in the next step without purification (150 mg, 0.94, 82% yield).

Step B

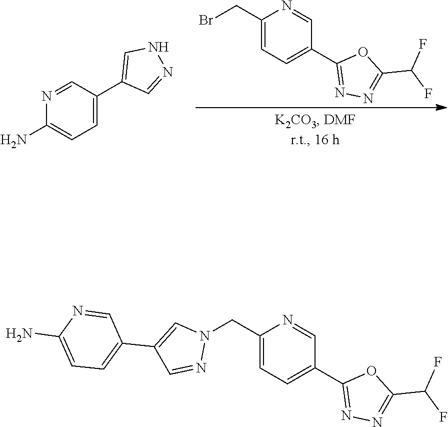 [SMILES: Nc1ccc(-c2cn[nH]c2)cn1 ; FC(F)c1nnc(-c2ccc(CBr)nc2)o1 ; Nc1ccc(-c2cnn(Cc3ccc(-c4nnc(C(F)F)o4)cn3)c2)cn1]

A mixture of 5-(1H-pyrazol-4-yl)pyridin-2-amine (75 mg, 0.47 mmol, 1 equiv.) and potassium carbonate (2 equiv.) in 3 mL DMF was stirred at r.t. over 20 min. 2-(6-(bromomethyl)pyridin-3-yl)-5-(difluoromethyl)-1,3,4-oxadiazole (Intermediate A, 1 equiv.) was added and the reaction mixture was stirred overnight. Full conversion of the starting bromide was confirmed by LC-MS. The reaction mixture was concentrated and submitted to prep-HPLC to give target compound (16.5 mg, 0.044, 9% yield, m/z 370.97 [MH+]).

### Example 63. Synthesis of N-(3-(1-((5-(5-(difluoromethyl)-1,3,4-oxadiazol-2-yl)pyridin-2-yl)methyl)-1H-pyrazol-4-yl)phenyl)morpholine-4-carboxamide (Compd. 119)

Step A

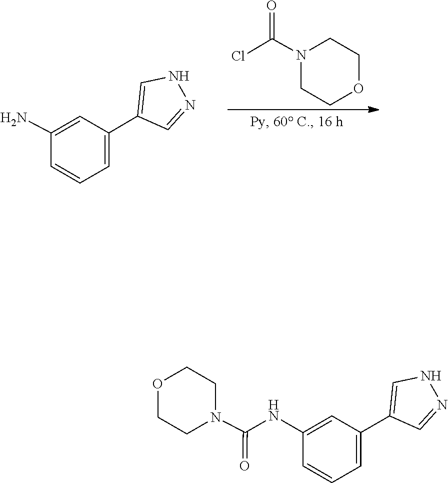 [SMILES: Nc1cccc(-c2cn[nH]c2)c1 ; O=C(Cl)N1CCOCC1 ; O=C(Nc1cccc(-c2cn[nH]c2)c1)N1CCOCC1]

3-(1H-pyrazol-4-yl)aniline (250 mg, 1.57 mmol, 1 equiv.) was dissolved in 5 mL pyridine and morpholine-4-carbonyl chloride (1.2 equiv.) was added. The reaction mixture was heated to 60° C. and stirred overnight. All starting material was consumed, but the desired product represented only 25% of the obtained mixture. The reaction mixture was evaporated, dissolved in water, acidified to pH=3 and extracted with EtOAc.

Crude product was purified by flash column chromatography (0-10% MeOH/DCM)

Step B

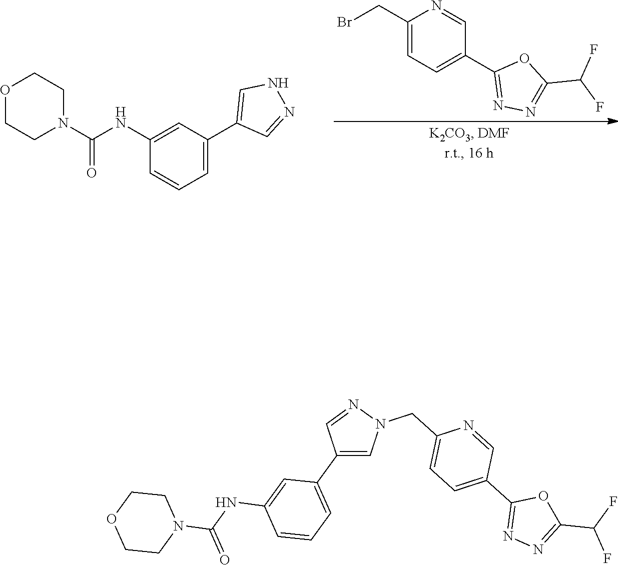 [SMILES: O=C(Nc1cccc(-c2cn[nH]c2)c1)N1CCOCC1 ; FC(F)c1nnc(-c2ccc(CBr)nc2)o1 ; O=C(Nc1cccc(-c2cnn(Cc3ccc(-c4nnc(C(F)F)o4)cn3)c2)c1)N1CCOCC1]

N-[3-(1H-pyrazol-4-yl)phenyl]morpholine-4-carboxamide (44 mg, 0.13 mmol, 1 equiv.) was suspended in 1 mL DMF, and potassium carbonate (1 equiv.) was added. The reaction mixture was stirred for 1 h, then 2-[6-(bromomethyl)pyridin-3-yl]-5-(difluoromethyl)-1,3,4-oxadiazole (Intermediate A, 1 equiv.) was added. The mixture was stirred at r.t. overnight, then diluted with EtOAc and washed with sat. aq. NaHCO₃ (2×) and brine. Organic phase was dried over Na₂SO₄, filtered and evaporated to give a crude product, which was purified by prep-HPLC (C18, ACN/water) to obtain pure title compound (10 mg, 0.02 mmol, 8% yield, m/z 481.92 [MH+]).

The following compounds were synthesized according to the same procedure:

| Compd. | Structure | m/z [MH+] |
|---|---|---|
| 227 | 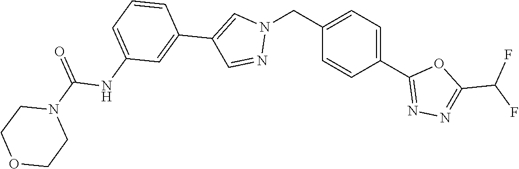 [SMILES: O=C(Nc1cccc(-c2cnn(Cc3ccc(-c4nnc(C(F)F)o4)cc3)c2)c1)N1CCOCC1] | 480.98 |
| 333 | 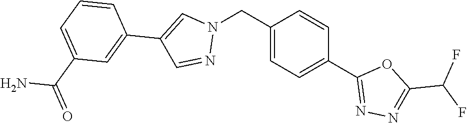 [SMILES: NC(=O)c1cccc(-c2cnn(Cc3ccc(-c4nnc(C(F)F)o4)cc3)c2)c1] | 396.17 |
| 334 | 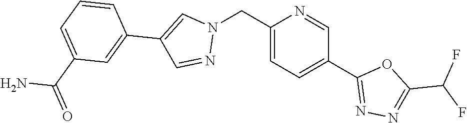 [SMILES: NC(=O)c1cccc(-c2cnn(Cc3ccc(-c4nnc(C(F)F)o4)cn3)c2)c1] | 396.93 |

### Example 64. Synthesis of N-(4-(1-(4-(5-(difluoromethyl)-1,3,4-oxadiazol-2-yl)benzyl)-1H-pyrazol-4-yl)phenyl)-4,5-dihydro-1H-imidazol-2-amine (Compd. 269)

Step A

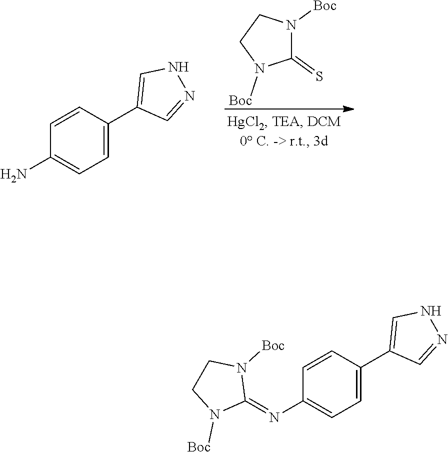 [SMILES: Nc1ccc(-c2cn[nH]c2)cc1 ; CC(C)(C)OC(=O)N1CCN(C(=O)OC(C)(C)C)C1=S ; CC(C)(C)OC(=O)N1CCN(C(=O)OC(C)(C)C)C1=Nc1ccc(-c2cn[nH]c2)cc1]

Mercury(II) chloride (1.1 equiv.) was added to a solution of 4-(1H-imidazol-4-yl)aniline (242 mg, 1.52 mmol, 1 equiv.), di-tert-butyl 2-thioxoimidazolidine-1,3-dicarboxylate (1 equiv.) and triethylamine (3.1 equiv.) in 10 mL DCM at 0° C. The resulting mixture was stirred at 0° C. over 1 h, then allowed to reach r.t. and stirred over 3 days. The reaction mixture was diluted with water and DCM. The layers were separated, and the organic phase was filtered. The filtrate was washed with sat. aq. NaHCO₃, brine, dried over Na₂SO₄, filtered, and concentrated under reduced pressure. The resulting beige solid was used in the next step without further purification (649 mg, 1.52, 100% yield).

Step B

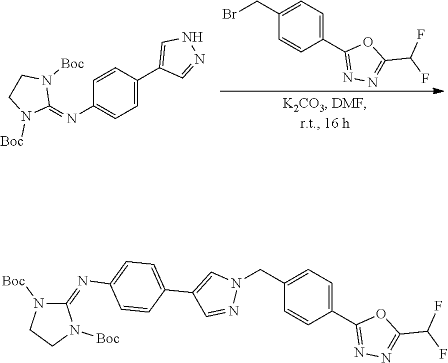 [SMILES: CC(C)(C)OC(=O)N1CCN(C(=O)OC(C)(C)C)C1=Nc1ccc(-c2cn[nH]c2)cc1 ; FC(F)c1nnc(-c2ccc(CBr)cc2)o1 ; CC(C)(C)OC(=O)N1CCN(C(=O)OC(C)(C)C)C1=Nc1ccc(-c2cnn(Cc3ccc(-c4nnc(C(F)F)o4)cc3)c2)cc1]

di-tert-butyl 2-((4-(1H-pyrazol-4-yl)phenyl)imino)imidazolidine-1,3-dicarboxylate (300 mg, 0.70 mmol, 1 equiv.) and potassium carbonate (1.1 equiv.) were suspended in 2.5 mL DMF. After 15 min 2-(4-(bromomethyl)phenyl)-5-(difluoromethyl)-1,3,4-oxadiazole (Intermediate B, 1 equiv.) was added to the resulting suspension and the reaction mixture was stirred at r.t. overnight. Water was added to the reaction mixture, which was extracted with ethyl acetate. The organic phase was washed with sat. aq. NaHCO₃ and brine, dried over Na₂SO₄ and filtered. After concentration under reduced pressure, the residue was purified by flash column chromatography (hexane/EtOAc 3:7 to 5:95) affording the product as a yellow solid (240 mg, 0.38 mmol, 54% yield).

Step C

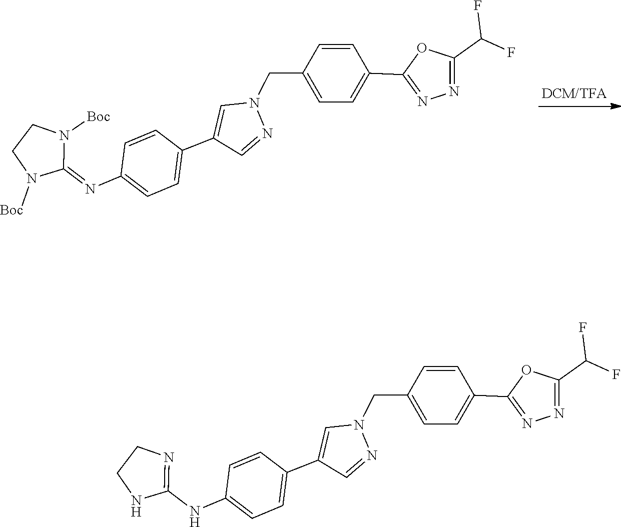 [SMILES: CC(C)(C)OC(=O)N1CCN(C(=O)OC(C)(C)C)C1=Nc1ccc(-c2cnn(Cc3ccc(-c4nnc(C(F)F)o4)cc3)c2)cc1 ; FC(F)c1nnc(-c2ccc(Cn3cc(-c4ccc(NC5=NCCN5)cc4)cn3)cc2)o1]

di-tert-butyl 2-((4-(1-(4-(5-(difluoromethyl)-1,3,4-oxadiazol-2-yl)benzyl)-1H-pyrazol-4-yl)phenyl)imino)imidazolidine-1,3-dicarboxylate (240 mg, 0.38 mmol, 1 equiv.) was dissolved in 2.5 mL DCE and TFA (15 equiv.) was added. The reaction mixture was stirred at r.t. overnight. Full conversion was observed by LC-MS. The reaction mixture was concentrated under reduced pressure, and the residue thus obtained was dissolved in acetonitrile and concentrated under reduced pressure (3 times). The dark red oily residue was purified by prep-HPLC affording the desired product as a white solid, in formate form. This formate salt (22 mg) was dissolved in water/acetonitrile and solid sodium bicarbonate was added (pH slightly basic). Precipitation occurred upon stirring. The precipitate was collected by centrifugation and washed with a minimum amount of water and dried. The product was obtained as a free base after lyophilization (15 mg, 0.03 mmol, 9% yield, m/z 436.11 [MH+]).

The following compound was synthesized according to the same procedure:

| Compd. | Structure | m/z [MH+] |
|---|---|---|
| 270 | 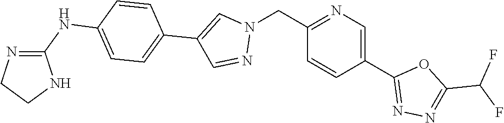 [SMILES: FC(F)c1nnc(-c2ccc(Cn3cc(-c4ccc(NC5=NCCN5)cc4)cn3)nc2)o1] | 437.13 |

### Example 65. Synthesis of 5-(5-((4-(5-(difluoromethyl)-1,3,4-oxadiazol-2-yl)phenyl)thio)-4-methyl-4H-1,2,4-triazol-3-yl)pyridin-2-amine (Compd. 285)

Step A

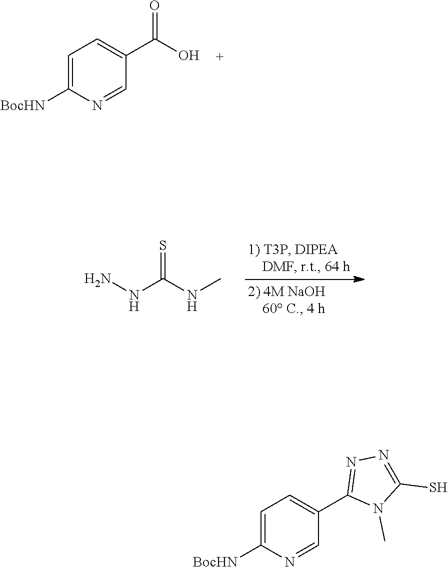

6-((tert-butoxycarbonyl)amino)nicotinic acid (300 mg, 1.47 mmol, 1 equiv.) and 4-methyl-3-thiosemicarbazide (1.1 equiv.) were suspended in DMF. T3P (1.5 equiv., 50% solution in DMF) and DIPEA (1.8 equiv.) were added, and the reaction mixture was stirred at r.t. over 64 h. LC-MS confirmed the formation of the reaction intermediate. The reaction mixture was diluted with EtOAc and water, then 4M NaOH was added. Aqueous phase was separated, organic layer was washed with 4M NaOH. Aqueous layers were collected together and stirred at 60° C. over 4 h. The white solid which formed was collected by filtration. The crude product thus obtained was used in the subsequent step without any further purification (230 mg, 0.75 mmol, 59% yield).

Step B

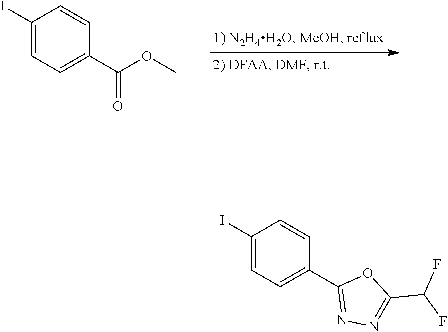

Methyl 4-iodobenzoate (5.07 g, 19.3 mmol, 1 equiv.) was dissolved in MeOH (25 mL), then hydrazine monohydrate was added (5 equiv.) under stirring. Mixture was refluxed over 3 h. Full conversion of methyl ester was observed by LC-MS (and TLC). The reaction mixture was concentrated and dried under vacuum. The white solid obtained (4.37 g) was dissolved in 10 mL of dry DMF and DFAA (3.5 equiv.) was added. The reaction mixture was stirred at 70° C. for 3 h. LC-MS confirmed full conversion of the starting material to product. A white precipitate formed upon dilution of the mixture with water. This solid was collected by filtration, rinsed with water and dried on air overnight. The obtained solid was suspended in 60 mL chloroform, filtered and rinsed with fresh chloroform twice. The filtrate was concentrated and the residue was dried under vacuum to obtain the desired product (3.5 g, 9.8 mmol, 51% yield).

Step C

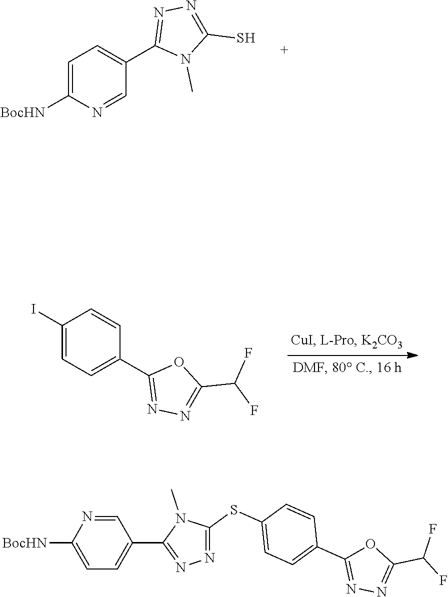 [SMILES: Cn1c(S)nnc1-c1ccc(NC(=O)OC(C)(C)C)nc1 ; FC(F)c1nnc(-c2ccc(I)cc2)o1 ; Cn1c(Sc2ccc(-c3nnc(C(F)F)o3)cc2)nnc1-c1ccc(NC(=O)OC(C)(C)C)nc1]

Copper iodide (0.05 equiv.), L-proline (0.1 equiv.) and potassium carbonate (1.11 equiv.) were dissolved in 3 mL DMF. The reaction mixture was degassed, and then 2-(difluoromethyl)-5-(4-iodophenyl)-1,3,4-oxadiazole (115 mg, 0.358 mmol, 1.1 equiv.) and tert-butyl (5-(5-mercapto-4-methyl-4H-1,2,4-triazol-3-yl)pyridin-2-yl)carbamate (100 mg, 0.325 mmol, 1 equiv.) were added under Ar. The reaction mixture was stirred at 80° C. overnight, and then diluted with water. A yellow solid precipitated (70% of desired product). The crude product thus obtained (87 mg, 0.17 mmol, 53% yield) was used directly in the next step.

Step D

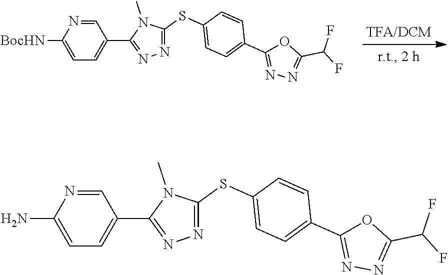 [SMILES: Cn1c(Sc2ccc(-c3nnc(C(F)F)o3)cc2)nnc1-c1ccc(NC(=O)OC(C)(C)C)nc1 ; Cn1c(Sc2ccc(-c3nnc(C(F)F)o3)cc2)nnc1-c1ccc(N)nc1]

tert-butyl (5-(5-((4-(5-(difluoromethyl)-1,3,4-oxadiazol-2-yl)phenyl)thio)-4-methyl-4H-1,2,4-triazol-3-yl)pyridin-2-yl)carbamate (87 mg, 0.17 mmol, 1 equiv.) was dissolved in 1 mL DCM. TFA (10 equiv.) was added, and the reaction mixture was stirred at r.t. over 2 h. DCM was added and the mixture was washed with sat. aq. NaHCO₃ (2×). Organic phase was separated, dried over Na₂SO₄, filtered and evaporated. Crude product was purified by prep-HPLC (0.1% FA/ACN/water C-18) to obtain 34 mg (0.085 mmol, 49% yield) of the title compound (m/z 402.0 [MH+]).

### Example 66. Synthesis of 5-(5-((4-(5-(difluoromethyl)-1,3,4-oxadiazol-2-yl)phenyl)thio)-4-methyl-4H-1,2,4-triazol-3-yl)pyridin-2-amine (Compd. 284)

Step A

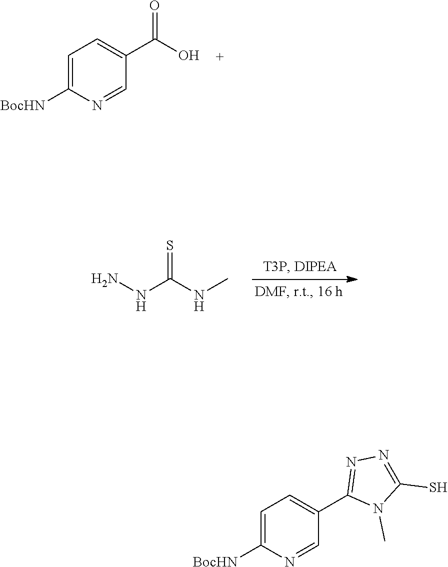

6-((tert-butoxycarbonyl)amino)nicotinic acid (300 mg, 1.47 mmol, 1 equiv.) and 4-methyl-3-thiosemicarbazide (1.1 equiv.) were suspended in DMF. T3P (1.5 equiv., 50% solution in DMF) and DIPEA (1.8 equiv.) were added, and the reaction mixture was stirred at r.t. over 64 h. LC-MS confirmed the formation of the reaction intermediate. The reaction mixture was diluted with EtOAc and water, then 4M NaOH was added. Aqueous phase was separated, organic layer was washed with 4M NaOH. Aqueous layers were collected together and stirred at 60° C. over 4 h. The white solid which formed was collected by filtration. The crude product thus obtained was used in the subsequent step without any further purification (230 mg, 0.75 mmol, 59% yield).

Step B

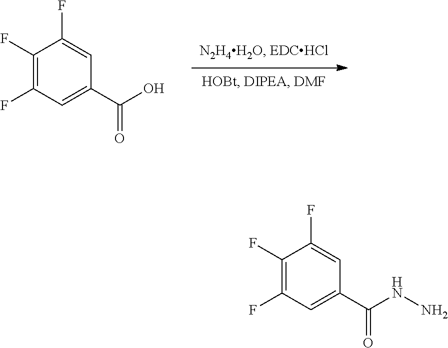 [SMILES: O=C(O)c1cc(F)c(F)c(F)c1 ; NNC(=O)c1cc(F)c(F)c(F)c1]

3,4,5-trifluorobenzoic acid (2 g, 11.3 mmol, 1 equiv.), 1-(3-dimethylaminopropyl)-3-ethylcarbodiimide hydrochloride (1.4 equiv.) and HOBt (1.4 equiv.) were dissolved in 10 mL anhydrous DMF. N,N-diisopropylethylamine (6 equiv.) was added, and the reaction mixture was stirred at r.t. for 20 min. The solution was cooled down to 0° C. with an ice bath and hydrazine monohydrate (5 equiv.) was added in one portion. The resulting mixture was stirred over 30 min at 0° C., then allowed to reach r.t. and stirred overnight. Product formation was monitored by LC-MS. The reaction mixture was diluted with water and the forming precipitate was filtered off. The filtrate was extracted with MTBE to obtain the desired product (1.6 g, 5.9 mmol, 52% yield).

Step C

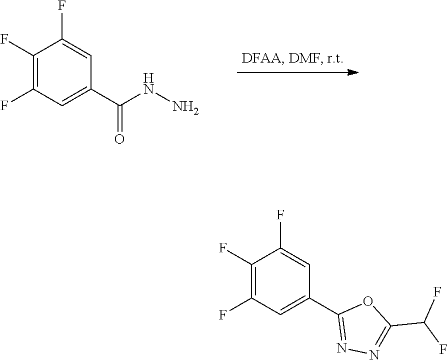 [SMILES: NNC(=O)c1cc(F)c(F)c(F)c1 ; Fc1cc(-c2nnc(C(F)F)o2)cc(F)c1F]

3,4,5-trifluorobenzohydrazide (1.6 g, 5.9 mmol, 1 equiv.) was dissolved in 10 mL of DMF and DFAA (4 equiv.) was added. The reaction mixture was stirred at 70° C. for 3 h. LC-MS confirmed full conversion of the starting material to product. A white precipitate formed upon dilution of the mixture with water. This solid was collected by filtration, rinsed with water and dried on air overnight. The obtained solid was suspended in 60 mL of chloroform, filtered and rinsed with fresh chloroform twice. The filtrate was concentrated and the residue was dried under vacuum to obtain the desired product (1.47 g, 5.3 mmol, 90% yield).

Step D

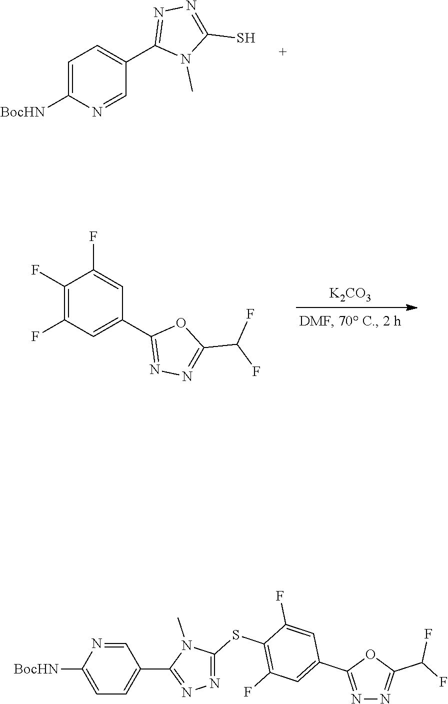

tert-butyl N-[5-(4-methyl-5-sulfanyl-1,2,4-triazol-3-yl)pyridin-2-yl]carbamate (80 mg, 0.26 mmol, 1 equiv.), 2-(difluoromethyl)-5-(3,4,5-trifluorophenyl)-1,3,4-oxadiazole (72 mg, 0.29 mmol, 1.1 equiv.) and potassium carbonate (2.2 equiv.) were suspended in 3 mL DMF. The reaction mixture was heated at 70° C. over 2 h. A yellow solid precipitated upon dilution with water. Collection of the solid by filtration gave the desired product (83 mg, 0.15 mmol, 59% yield).

Step E

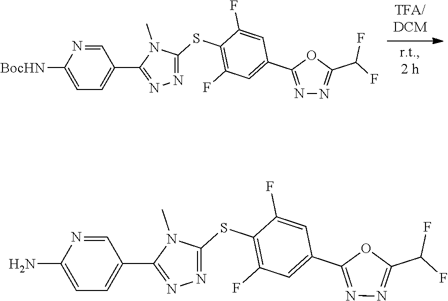 [SMILES: Cn1c(Sc2c(F)cc(-c3nnc(C(F)F)o3)cc2F)nnc1-c1ccc(NC(=O)OC(C)(C)C)nc1 ; Cn1c(Sc2c(F)cc(-c3nnc(C(F)F)o3)cc2F)nnc1-c1ccc(N)nc1]

tert-butyl N-[5-[5-[4-[5-(difluoromethyl)-1,3,4-oxadiazol-2-yl]-2,6-difluorophenyl]sulfanyl-4-methyl-1,2,4-triazol-3-yl]pyridin-2-yl]carbamate (83 mg, 0.154 mmol, 1 equiv.) was dissolved in 2 mL DCM. TFA (10 equiv.) was added, and the reaction mixture was stirred at r.t. over 2 h. DCM was added and the mixture was washed with sat. aq. NaHCO₃ (2×). Organic phase was separated, dried over Na₂SO₄, filtered and evaporated. Crude product was purified by prep-HPLC (0.1% FA/ACN/water C-18) affording 20 mg (0.045 mmol, 29% yield) of the title compound (m/z 438.0 [MH+]).

### Example 67. Synthesis of 2-(4-(bromomethyl-d2)-2,3-difluorophenyl)-5-(difluoromethyl)-1,3,4-oxadiazole (Intermediate R)

Step A

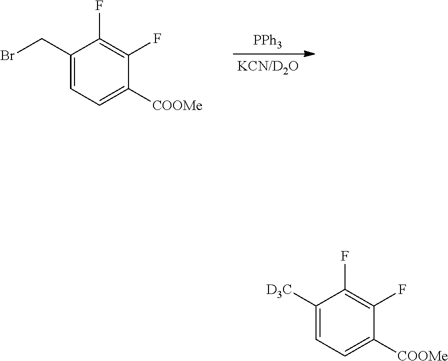 [SMILES: COC(=O)c1ccc(CBr)c(F)c1F ; [2H]C([2H])([2H])c1ccc(C(=O)OC)c(F)c1F ; "PPh3"]

Triphenylphosphine (1.2 equiv.) was added to a solution of methyl 4-(bromomethyl)-2,3-difluorobenzoate (1.23 g, 4.6 mmol, 1 equiv.) in a 1:1 mixture of D20/THF. The mixture was stirred at r.t. overnight. Potassium cyanide (1.2 equiv.) was then added to the reaction mixture, which was stirred at r.t. overnight. The mixture was extracted with EtOAc, dried over Na₂SO₄ and concentrated under reduced pressure. The crude residue was purified by flash column chromatography (Hex:EtOAc), to obtain the desired product (763 mg, 4.03 mmol, 87% yield).

Step B

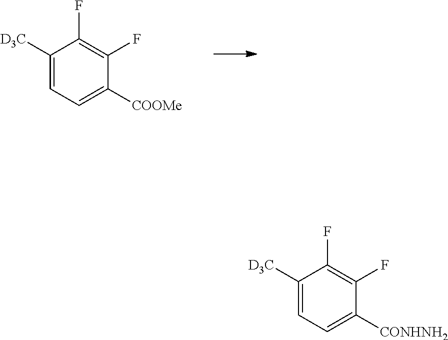

Methyl 2,3-difluoro-4-(methyl-d3)benzoate (763 mg, 4.03 mmol) was dissolved in MeOH (11 mL). Hydrazine monohydrate (5 equiv.) was added and the resulting mixture was stirred at 60° C. over 3 h. The reaction mixture was evaporated and the residue (740 mg, 3.91 mmol, 97% yield) was used directly in the subsequent step.

Step C

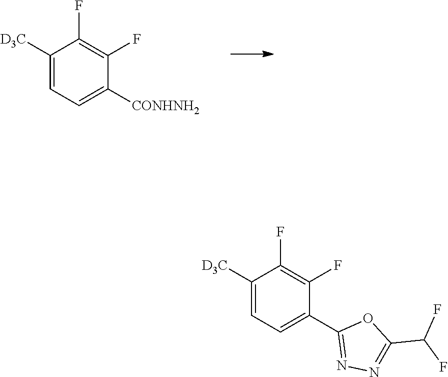

DFAA (2.5 equiv.) was added to a solution of 2,3-difluoro-4-(trideuteriomethyl)benzohydrazide (740 mg, 3.91 mmol, 1 equiv.) in 15 mL DMF. The mixture thus obtained was stirred for 3 h. Then, 0.5 extra equiv. of DFAA was added and the mixture was stirred overnight. The reaction mixture was poured into sat. aq. NaHCO₃ solution and then extracted using EtOAc. Organic layers were collected together, dried over Na₂SO₄, filtered and concentrated to give a crude product, which was purified by flash column chromatography (Hex:EtOAc/85:15). (529 mg, 2.12 mmol, 54% yield)

Step D

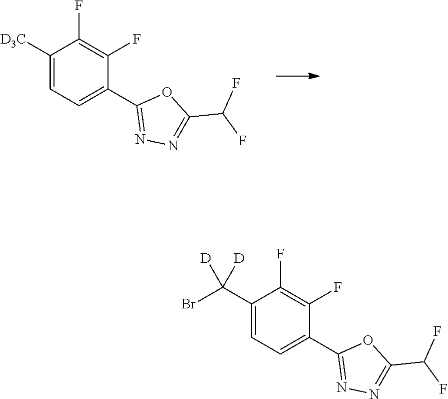 [SMILES: [2H]C([2H])([2H])c1ccc(-c2nnc(C(F)F)o2)c(F)c1F ; [2H]C([2H])(Br)c1ccc(-c2nnc(C(F)F)o2)c(F)c1F]

2-(difluoromethyl)-5-[2,3-difluoro-4-(trideuteriomethyl)phenyl]-1,3,4-oxadiazole (529 mg, 2.12 mmol, 1 equiv.) was dissolved in carbon tetrachloride (7 mL). Then, NBS (1.55 equiv.) and AIBN (0.1 eq) were added. The reaction mixture was degassed and refluxed (75° C.) under argon atmosphere for 3 h. Then, NBS (0.5 eq) and AIBN (0.05 eq) were added. The reaction mixture was degassed and refluxed (75° C.) under argon atmosphere for 5 h. The reaction mixture was cooled down, diluted with DCM, washed with water twice, then with aq. sodium thiosulfate and with aq. NaHCO₃. The organic phase was dried over Na₂SO₄, filtered and evaporated under reduced pressure. The crude residue was purified using flash column chromatography (Hex:EtOAc) to obtain the pure product in 42% yield (291 mg, 0.89 mmol). The following building blocks were prepared following the same procedure:

| Compd. | Structure |
|---|---|
| Intermediate S | 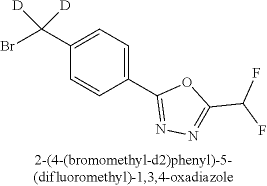 [SMILES: [2H]C([2H])(Br)c1ccc(-c2nnc(C(F)F)o2)cc1] |
| Intermediate T | 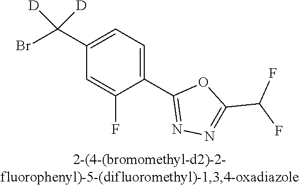 [SMILES: [2H]C([2H])(Br)c1ccc(-c2nnc(C(F)F)o2)c(F)c1] |
| Intermediate U | 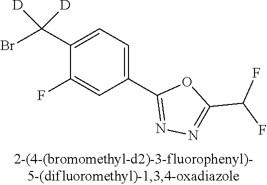 [SMILES: [2H]C([2H])(Br)c1ccc(-c2nnc(C(F)F)o2)cc1F] |
| Intermediate V | 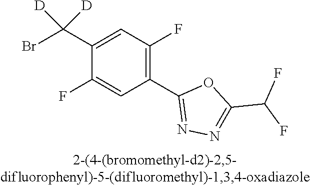 [SMILES: [2H]C([2H])(Br)c1cc(F)c(-c2nnc(C(F)F)o2)cc1F] |

### Example 68. Synthesis of 2-(6-(bromomethyl-d2)pyridin-3-yl)-5-(difluoromethyl)-1,3,4-oxadiazole (Intermediate X)

Step A

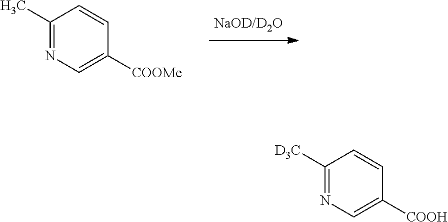

Methyl 6-methylnicotinate (1.76 g, 11.6 mmol, 1 equiv.) was dissolved in deuterium oxide and sodium deuteroxide (40% wt in D2O, 3.6 equiv.) was added. The reaction mixture was stirred at 140° C. for 1 h under MW irradiation. Solvent was evaporated and the crude product was used directly in the next step without any further purification (2.46 g, 10.6 mmol, 91% yield).

Step B

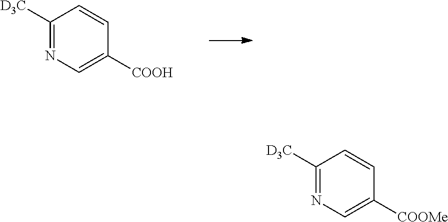 [SMILES: [2H]C([2H])([2H])c1ccc(C(=O)O)cn1 ; [2H]C([2H])([2H])c1ccc(C(=O)OC)cn1]

The crude 6-(methyl-d3)nicotinic acid (4.2 g, 29.7 mmol, 1 equiv.) was dissolved in 150 mL MeOH. The mixture was cooled down to 0° C. with an ice bath and SOCl₂ (10 equiv.) was added dropwise. Then the mixture was let to reach r.t. and was stirred overnight. The mixture was neutralized by adding sat. aq. NaHCO₃ and then pH was adjusted to 9 with 1 M NaOH solution. The product was extracted into EtOAc. The combined organic phases were washed with brine, dried over Na₂SO₄, filtered and carefully evaporated (product sublimates at low pressure) to obtain 1.5 g of crude product (9.7 mmol, 32.7% yield).

Step C

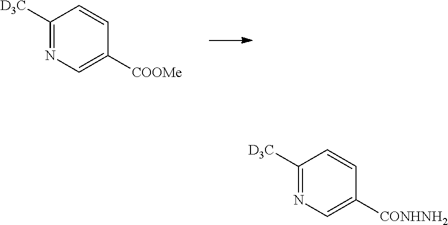

Hydrazine monohydrate (5 equiv.) was added to a solution of methyl 6-(methyl-d3)nicotinate (1.5 g, 9.7 mmol, 1 equiv.) in 39 mL MeOH, and the resulting mixture was stirred at 60° C. over 5 h. Extra 1.5 equiv. of hydrazine monohydrate was then added and the mixture was stirred overnight. Volatiles were evaporated, obtaining a crude product which was used in the subsequent step without any further purification (978 mg, 6.3 mmol, 65% yield).

Step D

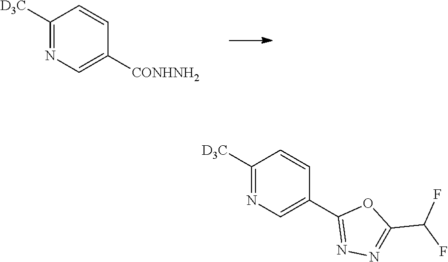

DFAA (2.5 equiv.) was added to a solution of 6-(methyl-d3)nicotinohydrazide (978 mg, 6.3 mmol, 1 equiv.) in 25 mL DMF, and the resulting mixture was stirred over 4 h. The reaction mixture was poured into sat. aq. NaHCO₃ and then extracted with EtOAc. Organic layers were collected together, dried over Na₂SO₄, filtered and evaporated to give a crude product, which was purified on flash column chromatography (Hex:EtOAc) (346 mg, 1.62 mmol, 25% yield).

Step E

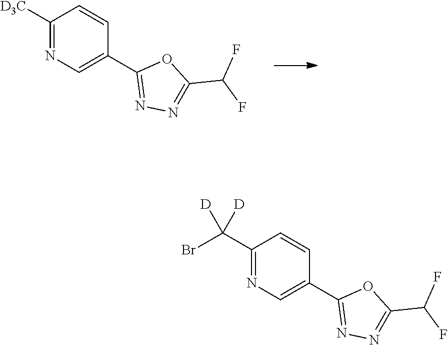 [SMILES: [2H]C([2H])([2H])c1ccc(-c2nnc(C(F)F)o2)cn1 ; [2H]C([2H])(Br)c1ccc(-c2nnc(C(F)F)o2)cn1]

2-(difluoromethyl)-5-(6-(methyl-d3)pyridin-3-yl)-1,3,4-oxadiazole (346 mg, 1.62 mmol, 1 equiv.) was dissolved in 6.5 mL carbon tetrachloride. Then, NBS (1.05 equiv.) and AIBN (0.01 eq) were added. The reaction mixture was degassed and refluxed (75° C.) under argon atmosphere for 5 h. Then, after adding extra AIBN (0.1 eq), the reaction mixture was degassed and refluxed (75° C.) under argon atmosphere for 3 h. The reaction mixture was cooled down, diluted with DCM, washed with water twice, then with aq. sodium thiosulfate and with aq. NaHCO₃. The organic phase was dried over Na₂SO₄, filtered, and evaporated under reduced pressure. The crude residue was purified using flash column chromatography (Hex:EtOAc) to obtain the pure product in 8% yield (38 mg, 0.13 mmol).

### Example 69. Synthesis of 6-(1-(4-(5-(difluoromethyl)-1,3,4-oxadiazol-2-yl)benzyl)-1H-pyrazol-4-yl)benzo[d]thiazol-2-amine (Compd. 326)

Step A

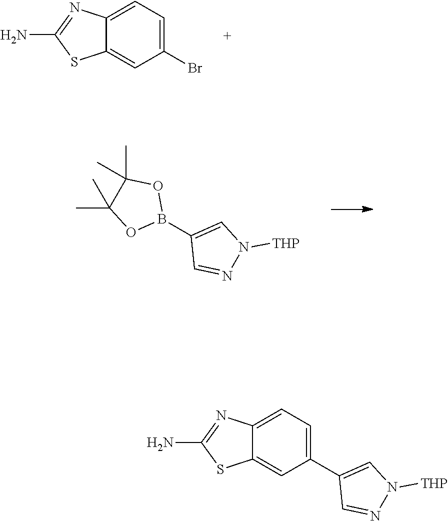

6-bromo-1,3-benzothiazol-2-amine (500 mg, 2.18 mmol, 1 equiv.), 1-(oxan-2-yl)-4-(4,4,5,5-tetramethyl-1,3,2-dioxaborolan-2-yl)pyrazole (1.3 equiv.) and cesium carbonate (3 equiv.) were dissolved in a 5:1 dioxane/water mixture. The resulting mixture was degassed with argon for 15 minutes. [1,1′-Bis(diphenylphosphino)ferrocene]dichloropalladium(II) dichloromethane (0.15 equiv.) was added and the reaction mixture was degassed with argon, sealed and stirred at 100° C. overnight. The mixture was then diluted with EtOAc and filtered through a pad of celite, washed with water (emulsion), sat. aq. NaHCO₃ and brine. The organic layer was then dried (MgSO₄), filtered and concentrated under reduced pressure. The residue was purified by flash chromatography (Hexane:EtOAc 1:1 to 5:95) affording the product as a red solid (210 mg, 0.7 mmol, 32% yield).

Step B

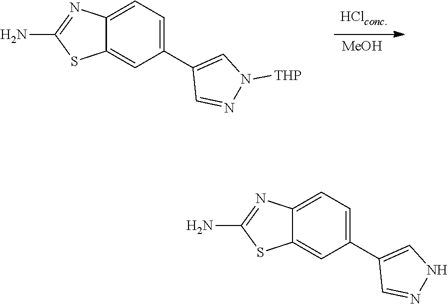

Concentrated HCl (20 equiv.) was added to a solution of 6-(1-(tetrahydro-2H-pyran-2-yl)-1H-pyrazol-4-yl)benzo[d]thiazol-2-amine (210 mg, 0.7 mmol, 1 equiv.) in 10 mL methanol. The reaction mixture was stirred at r.t. over 30 min. The reaction mixture was concentrated under reduced pressure and the residue was used directly in the next step (150 mg, 0.69 mmol, 99% yield).

Step C

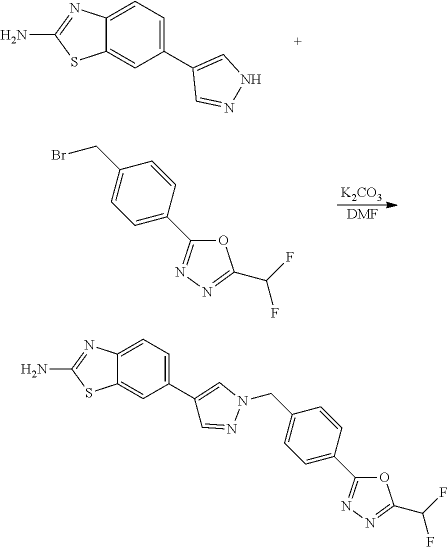

Potassium carbonate (2.5 equiv.) was added to a solution of 6-(1H-pyrazol-4-yl)benzo[d]thiazol-2-amine (25 mg, 0.116 mmol, 1 equiv.) in 1 mL DMF. After 15 min 2-(4-(bromomethyl)phenyl)-5-(difluoromethyl)-1,3,4-oxadiazole (Intermediate B, 1 equiv.) was added to the solution and the resulting mixture was stirred at r.t. overnight. Water was added to the reaction mixture, which was extracted into EtOAc. The organic layer was washed with sat. aq. NaHCO₃ and brine, dried (MgSO₄), filtered and concentrated under reduced pressure. The residue was purified by prep-HPLC (neutral conditions) to obtain the desired product (7 mg, 0.016 mmol, 14% yield, m/z 424.97 [M−H+]).

The following compounds were prepared according to the same procedure:

| Compd. | Structure | m/z [MH+] |
|---|---|---|
| 318 | 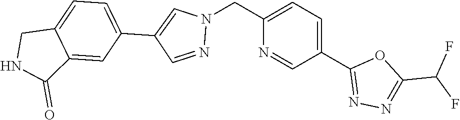 [SMILES: O=C1NCc2ccc(-c3cnn(Cc4ccc(-c5nnc(C(F)F)o5)cn4)c3)cc21] | 408.76 |
| 319 | 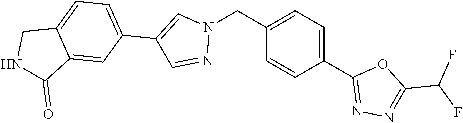 [SMILES: O=C1NCc2ccc(-c3cnn(Cc4ccc(-c5nnc(C(F)F)o5)cc4)c3)cc21] | 408.01 |
| 320 | 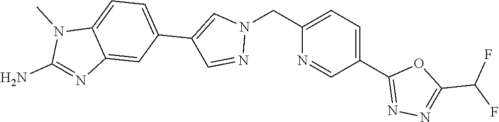 [SMILES: Cn1c(N)nc2cc(-c3cnn(Cc4ccc(-c5nnc(C(F)F)o5)cn4)c3)ccc21] | 423.02 |
| 321 | 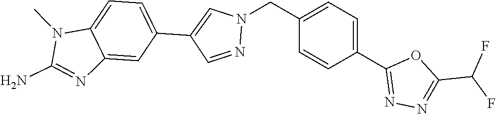 [SMILES: Cn1c(N)nc2cc(-c3cnn(Cc4ccc(-c5nnc(C(F)F)o5)cc4)c3)ccc21] | 422.04 |
| 327 | 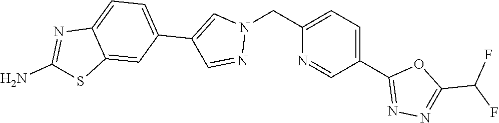 [SMILES: Nc1nc2ccc(-c3cnn(Cc4ccc(-c5nnc(C(F)F)o5)cn4)c3)cc2s1] | 425.97 |
| 331 | 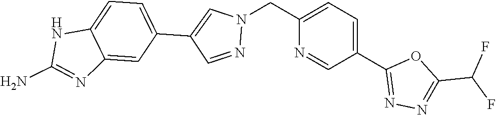 [SMILES: Nc1nc2cc(-c3cnn(Cc4ccc(-c5nnc(C(F)F)o5)cn4)c3)ccc2[nH]1] | 409.22 |
| 332 | 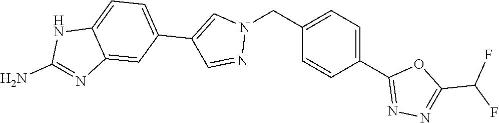 [SMILES: Nc1nc2cc(-c3cnn(Cc4ccc(-c5nnc(C(F)F)o5)cc4)c3)ccc2[nH]1] | 407.90 |

The following compounds were prepared according to the same procedure, starting from 1-(oxan-2-yl)-4-(4,4,5,5-tetramethyl-1,3,2-dioxaborolan-2-yl)pyrazole:

| Compd. | Structure | m/z [MH+] |
|---|---|---|
| 306 | 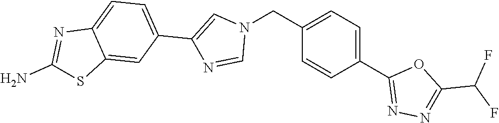 [SMILES: Nc1nc2ccc(-c3cn(Cc4ccc(-c5nnc(C(F)F)o5)cc4)cn3)cc2s1] | 425.03 |
| 307 | 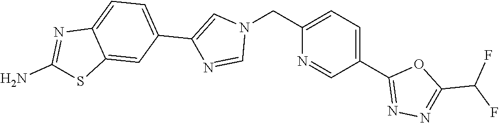 [SMILES: Nc1nc2ccc(-c3cn(Cc4ccc(-c5nnc(C(F)F)o5)cn4)cn3)cc2s1] | 426.08 |
| 322 | 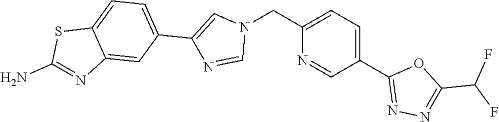 [SMILES: Nc1nc2cc(-c3cn(Cc4ccc(-c5nnc(C(F)F)o5)cn4)cn3)ccc2s1] | 425.95 |
| 328 | 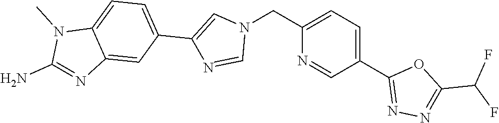 [SMILES: Cn1c(N)nc2cc(-c3cn(Cc4ccc(-c5nnc(C(F)F)o5)cn4)cn3)ccc21] | 422.93 |
| 329 | 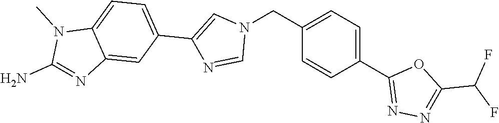 [SMILES: Cn1c(N)nc2cc(-c3cn(Cc4ccc(-c5nnc(C(F)F)o5)cc4)cn3)ccc21] | 422.05 |

### Example 70. Synthesis of 54 (4-(4-(4-(5-(difluoromethyl)-1,3,4-oxadiazol-2-yl)benzyl)-1H-1,2,3-triazol-1-yl)benzyl)amino)-2-methoxynicotinamide (Compd. 286)

Step A

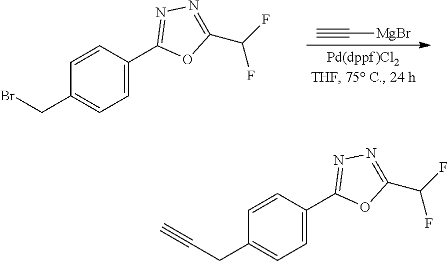

2-(4-(bromomethyl)phenyl)-5-(difluoromethyl)-1,3,4-oxadiazole (Intermediate B, 800 mg, 2.8 mmol, 1 equiv.) and [1,1′-Bis(diphenylphosphino)ferrocene]dichloropalladium(II) (0.1 equiv.) were added to a solution of ethynylmagnesium bromide (2.4 equiv.) in 8 mL THF at room temperature under argon. The reaction mixture was stirred at 75° C. over 24 h. Full conversion of the starting material was observed by LCMS. The reaction mixture was diluted with water, extracted with EtOAc, dried over magnesium sulfate, filtered, concentrated. The crude residue was purified by flash chromatography affording the desired product as a yellow solid (33 mg, 0.14 mmol, 5% yield).

Step B

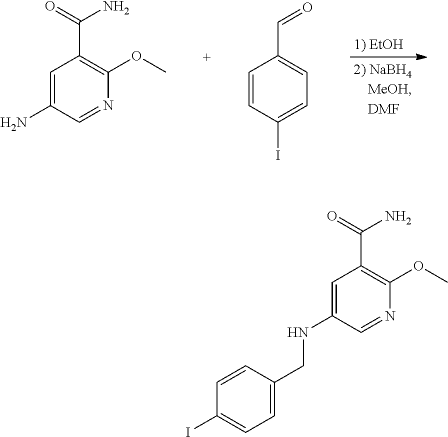 [SMILES: COc1ncc(N)cc1C(N)=O ; O=Cc1ccc(I)cc1 ; COc1ncc(NCc2ccc(I)cc2)cc1C(N)=O]

A mixture of 4-Iodobenzaldehyde (139 mg, 0.6 mmol, 1 equiv.) and 5-amino-2-methoxypyridine-3-carboxamide (100 mg, 0.6 mmol, 1 equiv.) in 3 mL ethanol was stirred at 70° C. overnight. The white precipitate which formed was collected by filtration and washed with ethanol.

Imine intermediate thus obtained (195 mg, 0.51 mmol, 1 equiv.) was suspended in 1 mL DMF and diluted with 6 mL methanol. Sodium borohydride (4 equiv.) was then added and the reaction mixture was stirred at r.t. overnight. A second portion of sodium borohydride (4 equiv.) was added a and the reaction mixture was stirred at r.t. overnight. The mixture was concentrated under reduced pressure and water was added to make the product precipitate as a white solid, which was collected by filtration and dried under vacuum (158 mg, 0.41 mmol, 90% yield).

Step C

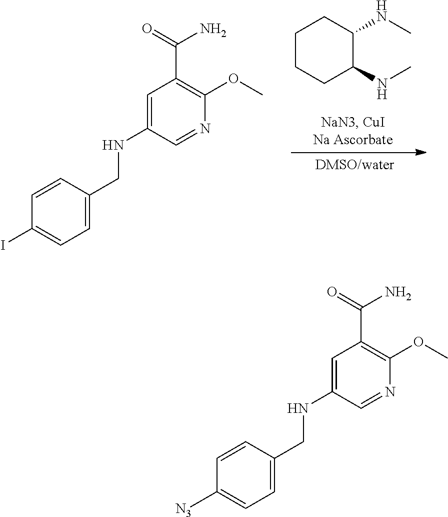

5-((4-iodobenzyl)amino)-2-methoxynicotinamide (175 mg, 0.46 mmol, 1 equiv.), sodium azide (2 equiv.), sodium ascorbate (0.05 equiv.), copper iodide (0.1 equiv.) and (S,S)-(+)-N,N′-dimethyl-1,2-cyclohexanediamine (0.15 equiv.) were dissolved in a 1:1 mixture DMSO/water. The reaction mixture was degassed with argon and stirred at r.t. overnight. The reaction mixture was diluted with water and the product was extracted with ethyl acetate. The organic layer was washed with brine, dried over magnesium sulfate, filtered and concentrated under reduced pressure to afford the product as a yellow solid (136 mg, 0.46 mmol, 100% yield).

Step D

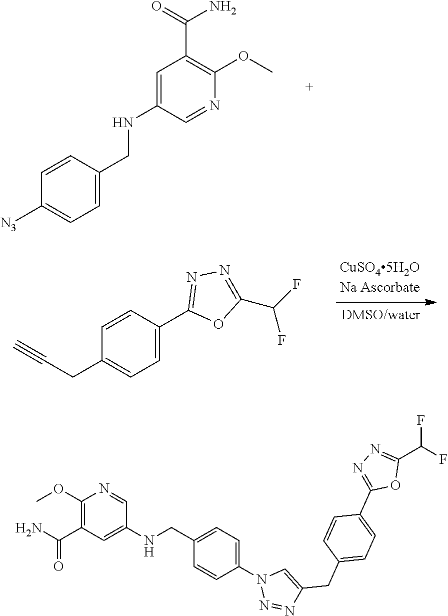

5-((4-azidobenzyl)amino)-2-methoxynicotinamide (19 mg, 0.063 mmol, 1 equiv.) and 2-(difluoromethyl)-5-(4-(prop-2-yn-1-yl)phenyl)-1,3,4-oxadiazole (15 mg, 0.063 mmol, 1 equiv.) were dissolved in 0.6 mL DMSO. Copper(II) sulfate pentahydrate (0.2 equiv., 0.04 M aqueous solution) and sodium L-ascorbate (0.4 equiv., 0.08 M aqueous solution) were added, and the mixture was stirred at 40° C. overnight.

The reaction mixture was diluted with water and extracted with ethyl acetate. The organic layer was washed with brine, dried over magnesium sulfate, filtered and concentrated under reduced pressure. The residue was purified by flash column chromatography (DCM/MeOH) and further purified by pTLC (DCM:Hexane:MeOH 4:4:0.5). 20 mg of the target product were obtained as light-yellow solid (0.037 mmol, 60% yield). (m/z 533.18 [MH+])

The following compounds were synthesized according to the same procedure:

6687 starting from 6-bromobenzo[d]thiazol-2-amine

| Compd. | Structure | m/z [MH+] |
|---|---|---|
| 377 | 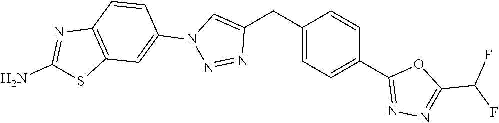 [SMILES: Nc1nc2ccc(-n3cc(Cc4ccc(-c5nnc(C(F)F)o5)cc4)nn3)cc2s1] | 426.1 |

### Example 71. Synthesis of N-(4-(4-(4-(5-(difluoromethyl)-1,3,4-oxadiazol-2-yl)benzyl)-1H-1,2,3-triazol-1-yl)phenyl)-4,5-dihydro-1H-imidazol-2-amine (Compd. 316)

Step A

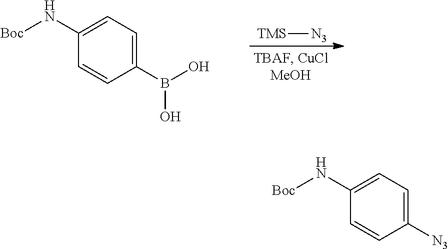 [SMILES: CC(C)(C)OC(=O)Nc1ccc(B(O)O)cc1 ; C[Si](C)(C)N=[N+]=[N-] ; CC(C)(C)OC(=O)Nc1ccc(N=[N+]=[N-])cc1]

Tetrabutylammonium fluoride (1.5 equiv.), trimethylsilyl azide (1.5 equiv.), and copper chloride (0.1 equiv.) were sequentially added to a solution of (4-((tert-butoxycarbonyl)amino)phenyl)boronic acid (2 g, 8.44 mmol, 1 equiv.) in 30 mL methanol. The reaction mixture was stirred at 65° C. Full conversion was observed after 24 h. The crude product was purified by flash chromatography (hexane/EtOAc 98:2 to 92:8) (1.48 g, 6.32 mmol, 75% yield).

Step B

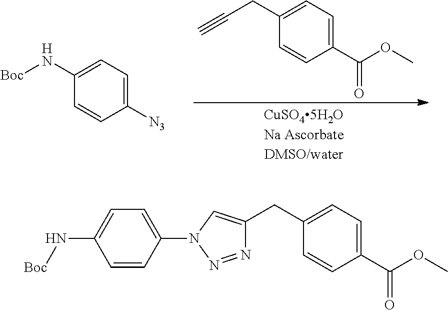 [SMILES: CC(C)(C)OC(=O)Nc1ccc(N=[N+]=[N-])cc1 ; C#CCc1ccc(C(=O)OC)cc1 ; COC(=O)c1ccc(Cc2cn(-c3ccc(NC(=O)OC(C)(C)C)cc3)nn2)cc1]

Tert-butyl (4-azidophenyl)carbamate (117 mg, 0.5 mmol, 1 equiv.) was dissolved in DMSO (2.5 mL). Methyl 4-(prop-2-yn-1-yl)benzoate (87 mg, 0.5 mmol, 1 equiv.) was added, followed by CuSO₄ pentahydrate (0.5M aq. sol., 0.2 equiv.) and sodium ascorbate (1M aq. sol., 0.4 equiv.). The resulting mixture was stirred at r.t. overnight. Water was added and the mixture was extracted with EtOAc (filtration over celite was necessary). The organic layer was washed with brine, dried over MgSO₄, filtered, and concentrated under reduced pressure. The residue was purified by flash chromatography (hexane/EtOAc 3:1 to 1:1) affording the desired product as a white solid (155 mg, 0.38 mmol, 75% yield).

Step C

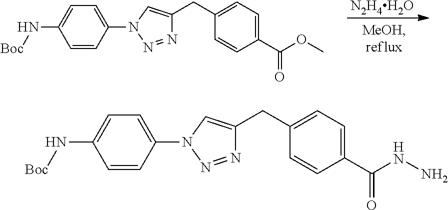

Methyl 4-((1-(4-((tert-butoxycarbonyl)amino)phenyl)-1H-1,2,3-triazol-4-yl)methyl)benzoate (287 mg, 0.7 mmol, 1 equiv.) was dissolved in 5 mL methanol and hydrazine hydrate (20 equiv.) was added. The reaction mixture was stirred at 75° C. over 3 days. Precipitation of the product occurred upon cooling the mixture to r.t. The white solid was collected by filtration and washed with a minimum amount of water. The product was dried overnight under reduced pressure and was used directly in the next step without any further purification (287 mg, 0.7 mmol, 100% yield).

Step D

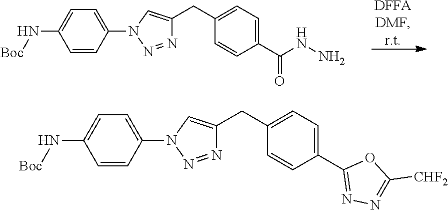

Tert-butyl (4-(4-(4-(hydrazinecarbonyl)benzyl)-1H-1,2,3-triazol-1-yl)phenyl)carbamate (287 mg, 0.7 mmol, 1 equiv.) was dissolved in DMF (4 mL) under argon. DFAA (10 equiv.) was added, the flask was sealed and the reaction mixture was stirred at r.t. over 3 days. The mixture was diluted with water (precipitation occurred) and extracted with EtOAc. The organic layers were washed with NaHCO₃, brine, dried (MgSO₄), filtered and concentrated under reduced pressure. The residue was purified by flash chromatography (hexane/EtOAc 2:1 to 1:1) affording the product as a white solid. (155 mg, 0.33 mmol, 47% yield)

Step E

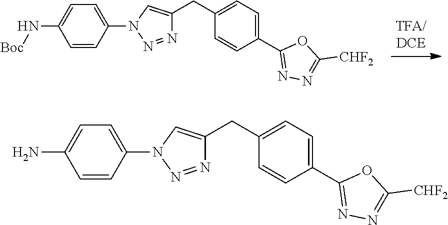 [SMILES: CC(C)(C)OC(=O)Nc1ccc(-n2cc(Cc3ccc(-c4nnc(C(F)F)o4)cc3)nn2)cc1 ; Nc1ccc(-n2cc(Cc3ccc(-c4nnc(C(F)F)o4)cc3)nn2)cc1]

Tert-butyl (4-(4-(4-(5-(difluoromethyl)-1,3,4-oxadiazol-2-yl)benzyl)-1H-1,2,3-triazol-1-yl)phenyl)carbamate (62 mg, 0.13 mmol, 1 equiv.) was dissolved in 1 mL DCE and TFA (12 equiv.) was added. The resulting mixture was stirred at r.t. over 4 h. The reaction mixture was then concentrated under reduced pressure. The residue was dissolved in acetonitrile and concentrated under reduced pressure (3 times) to remove excess TFA. The residue (brown oil) was used in the next step without any further treatments (TFA salt).

Step F

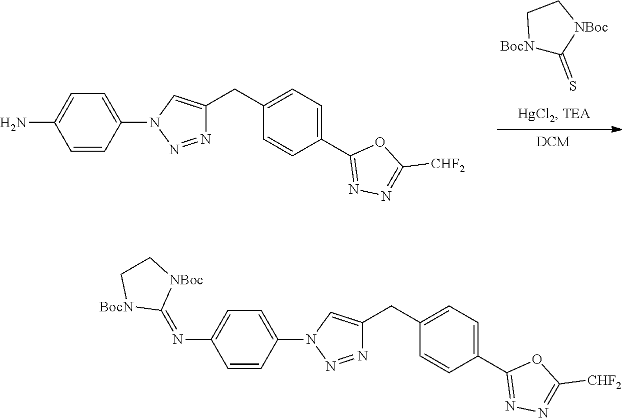 [SMILES: CC(C)(C)OC(=O)N1CCN(C(=O)OC(C)(C)C)C1=S ; Nc1ccc(-n2cc(Cc3ccc(-c4nnc(C(F)F)o4)cc3)nn2)cc1 ; CC(C)(C)OC(=O)N1CCN(C(=O)OC(C)(C)C)C1=Nc1ccc(-n2cc(Cc3ccc(-c4nnc(C(F)F)o4)cc3)nn2)cc1]

HgCl₂ (2.2 equiv.) was added to a solution of 4-(4-(4-(5-(difluoromethyl)-1,3,4-oxadiazol-2-yl)benzyl)-1H-1,2,3-triazol-1-yl)aniline (48 mg, 0.13 mmol, 1 equiv.), N,N′-di(tertbutoxycarbonyl)imidazolidine-2-thione (2 equiv.) and TEA (12 equiv.) in 1 mL DCM. The resulting mixture was stirred at r.t. over 4 days. The mixture was diluted with water and DCM, filtered and extracted with DCM. The organic layer was washed with brine, dried (MgSO₄), filtered, and concentrated under reduced pressure to afford a yellow solid which was used directly in the next step.

Step G

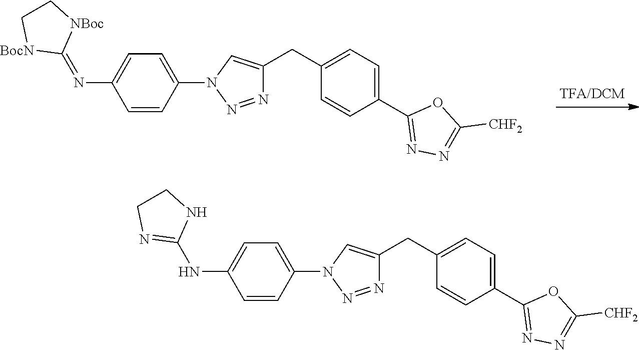 [SMILES: CC(C)(C)OC(=O)N1CCN(C(=O)OC(C)(C)C)C1=Nc1ccc(-n2cc(Cc3ccc(-c4nnc(C(F)F)o4)cc3)nn2)cc1 ; FC(F)c1nnc(-c2ccc(Cc3cn(-c4ccc(NC5=NCCN5)cc4)nn3)cc2)o1]

di-tert-butyl 2-((4-(4-(4-(5-(difluoromethyl)-1,3,4-oxadiazol-2-yl)benzyl)-1H-1,2,3-triazol-1-yl)phenyl)imino)imidazolidine-1,3-dicarboxylate (0.13 mmol, 1 equiv.) was dissolved in 1 mL DCM and TFA (0.8 mL, 80 equiv.) was added. The reaction mixture was stirred at r.t. for 2 h. The mixture was diluted with ethyl acetate and neutralized with NaHCO₃. The organic layer was separated and washed with brine, dried (MgSO₄), filtered and concentrated. The residue was purified by flash chromatography and the product was isolated as yellow solid. This was suspended in DCM, filtered (white suspension), concentrated to ˜1 mL. Hexane was added and the solid that formed was triturated and filtered to obtain a white solid, which was dried under vacuum (11 mg, 0.024 mmol, 18% yield over three steps). (m/z 436.77 [MH+])

The following compounds were prepared according to the same procedure:

| Compd. | Structure | m/z [MH+] |
|---|---|---|
| 315 | 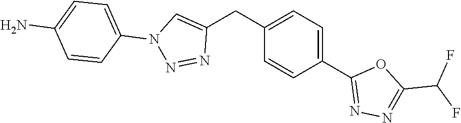 [SMILES: Nc1ccc(-n2cc(Cc3ccc(-c4nnc(C(F)F)o4)cc3)nn2)cc1] | 369.02 |
| 413 | 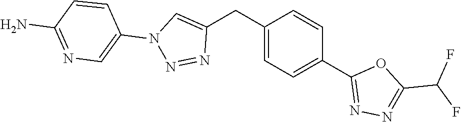 [SMILES: Nc1ccc(-n2cc(Cc3ccc(-c4nnc(C(F)F)o4)cc3)nn2)cn1] | 410.95* |
| 414 | 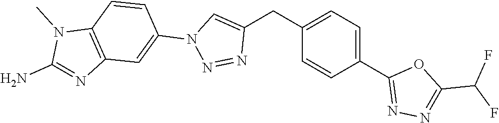 [SMILES: Cn1c(N)nc2cc(-n3cc(Cc4ccc(-c5nnc(C(F)F)o5)cc4)nn3)ccc21] | 423.21 |

*[M + ACN + H]⁺ was observed.

### Example 72. Synthesis of 4-(5-(4-(5-(difluoromethyl)-1,3,4-oxadiazol-2-yl)benzyl)oxazol-2-yl)aniline (Compd. 330)

Step A

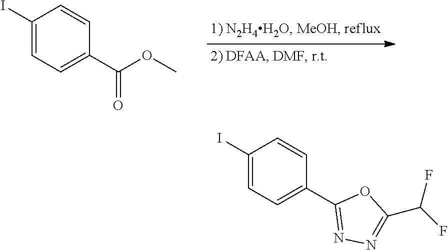

Methyl 4-iodobenzoate (5.07 g, 19.3 mmol, 1 equiv.) was dissolved in MeOH (25 mL), then hydrazine monohydrate was added (5 equiv.) under stirring. Mixture was refluxed over 3 h. Full conversion of methyl ester was observed by LC-MS (and TLC). The reaction mixture was concentrated and dried under vacuum. The white solid obtained (4.37 g) was dissolved in 10 mL of dry DMF and DFAA (3.5 equiv.) was added. The reaction mixture was stirred at 70° C. for 3 h. LC-MS confirmed full conversion of the starting material to product. A white precipitate formed upon dilution of the mixture with water. This solid was collected by filtration, rinsed with water and dried on air overnight. The obtained solid was suspended in 60 mL chloroform, filtered and rinsed with fresh chloroform twice. The filtrate was concentrated, and the residue was dried under vacuum to obtain the desired product (3.5 g, 9.8 mmol, 51% yield).

Step B

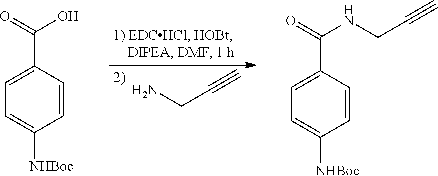

A mixture of 4-((tert-butoxycarbonyl)amino)benzoic acid (1 g, 4.21 mmol, 1 equiv.), EDC hydrochloride (1.3 equiv.), HOBt (1.3 equiv.) and DIPEA (2 equiv.) in 9 mL DMF was stirred at r.t. for 1 h. Then, propargylamine (1 equiv.) was added, and the resulting mixture was stirred at r.t. for 2 h. The reaction mixture was diluted with water and extracted with EtOAc. The combined organic layers were washed with sat. aq. NaHCO₃ and brine, dried over MgSO₄, filtered and concentrated under reduced pressure to give a yellow oil, which was used directly in the next step.

Step C

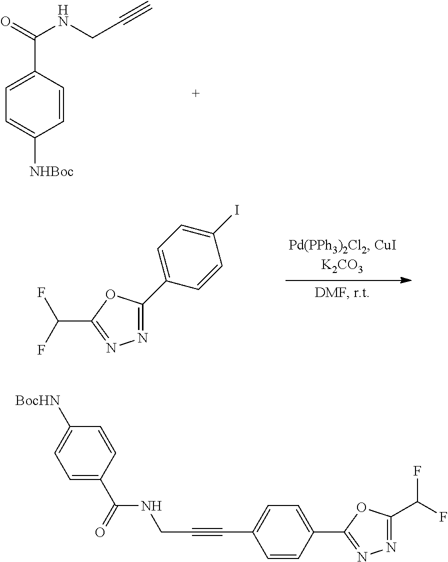 [SMILES: C#CCNC(=O)c1ccc(NC(=O)OC(C)(C)C)cc1 ; FC(F)c1nnc(-c2ccc(I)cc2)o1 ; CC(C)(C)OC(=O)Nc1ccc(C(=O)NCC#Cc2ccc(-c3nnc(C(F)F)o3)cc2)cc1]

A mixture of 2-(difluoromethyl)-5-(4-iodophenyl)-1,3,4-oxadiazole (150 mg, 0.47 mmol, 1 equiv.), Pd(PPh₃)₂Cl₂ (0.03 equiv.), copper iodide (0.06 equiv.) and potassium carbonate (2 equiv.) was stirred in 2.5 mL DMF at r.t. under argon. Then tert-butyl (4-(prop-2-yn-1-ylcarbamoyl)phenyl)carbamate (1.2 equiv.) was added and the resulting mixture was stirred at 70° C. overnight. Water was added to the reaction mixture, which was extracted with EtOAc. The organic layers were collected, dried over Na₂SO₄, filtered and concentrated. The crude residue obtained was used directly in the next step.

Step D

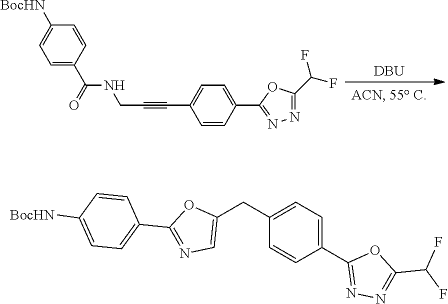

tert-butyl (4-((3-(4-(5-(difluoromethyl)-1,3,4-oxadiazol-2-yl)phenyl)prop-2-yn-1-yl)carbamoyl)phenyl)carbamate (218 mg, 0.46 mmol, 1 equiv.) was suspended in 4 mL acetonitrile and DBU (1 equiv.) was added. The reaction mixture was stirred at 55° C. overnight, then it was concentrated under reduced pressure. The residue was diluted with EtOAc and washed with 0.5M HCl aq. sol. and brine. The organic layer was concentrated under reduced pressure and the residue was purified by flash chromatography (hexane/EtOAc 7:3 to 1:1) affording the desired product (90 mg, 0.19 mmol, 41% yield).

Step E

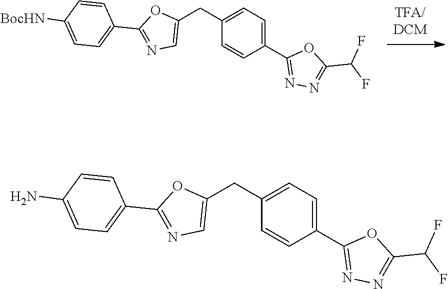 [SMILES: CC(C)(C)OC(=O)Nc1ccc(-c2ncc(Cc3ccc(-c4nnc(C(F)F)o4)cc3)o2)cc1 ; Nc1ccc(-c2ncc(Cc3ccc(-c4nnc(C(F)F)o4)cc3)o2)cc1]

tert-butyl (4-(5-(4-(5-(difluoromethyl)-1,3,4-oxadiazol-2-yl)benzyl)oxazol-2-yl)phenyl)carbamate (60 mg, 0.12 mmol, 1 equiv.) was dissolved in 1.2 mL DCM and TFA (15 equiv.) was added. The reaction mixture was stirred at r.t. for 2 h, and then it was concentrated under reduced pressure. The residue was suspended in acetonitrile and concentrated three times successively, in order to remove excess TFA. The residue was then dissolved with EtOAc and washed with NaHCO₃ and brine. The organic layer was concentrated under reduced pressure and the residue was purified by flash chromatography (hexane/EtOAc 1:1) affording the product as a yellow solid. This product was further purified by prep-HPLC (FA) affording the title compound as a white solid (9 mg, 0.024 mmol, 19% yield). (m/z 368.96 [MH+]).

### Example 73. Synthesis of N-(4-(5-(4-(5-(difluoromethyl)-1,3,4-oxadiazol-2-yl)-2-fluorobenzyl)-1,2,4-oxadiazol-3-yl)phenyl)-4,5-dihydro-1H-imidazol-2-amine (Compd. 420)

Step A

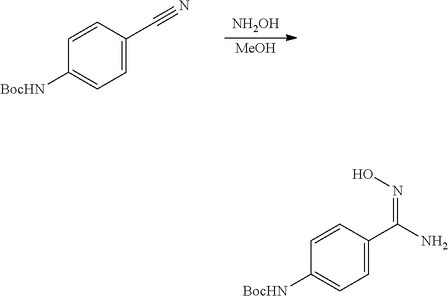

Hydroxylamine (50% wt. aq. sol., 3 equiv.) was added to a solution of tert-butyl (4-cyanophenyl)carbamate (5.32 g, 24.38 mmol, 1 equiv.) in 60 mL methanol. The resulting mixture was stirred at 70° C. overnight, and then it was filtered and concentrated under reduced pressure. The white solid thus obtained was dried under reduced pressure and used in the next step without any further purification (6.12 g, 24.37 mmol, 99% yield).

Step B

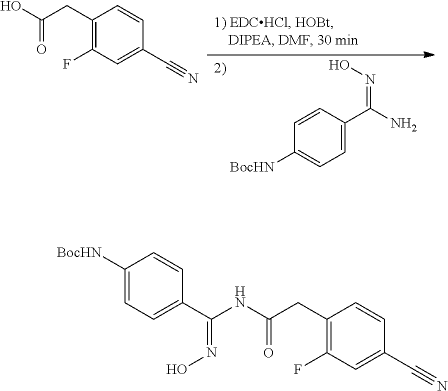 [SMILES: N#Cc1ccc(CC(=O)O)c(F)c1 ; CC(C)(C)OC(=O)Nc1ccc(/C(N)=N\O)cc1 ; CC(C)(C)OC(=O)Nc1ccc(/C(=N\O)NC(=O)Cc2ccc(C#N)cc2F)cc1]

A mixture of 2-(4-cyano-2-fluorophenyl)acetic acid (606 mg, 3.38 mmol, 1 equiv.), EDC hydrochloride (1.2 equiv.) and HOBt (1.2 equiv.) in 10 mL DMF was stirred at r.t. for 30 min. Then tert-butyl (E)-(4-(N′-hydroxycarbamimidoyl)phenyl)carbamate (1 equiv.) was added and the resulting mixture was stirred at r.t. over 2 days. Water (˜40 mL) was added to the reaction mixture. The white precipitate which formed was collected by filtration, washed with water, and dried under reduced pressure. The crude product thus obtained was used directly in the next step (255 mg, 0.62 mmol, 18% yield).

Step C

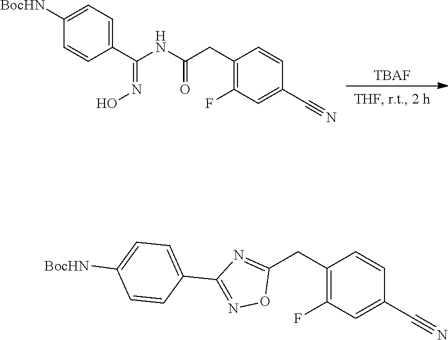 [SMILES: CC(C)(C)OC(=O)Nc1ccc(/C(=N\O)NC(=O)Cc2ccc(C#N)cc2F)cc1 ; "TBAF" ; CC(C)(C)OC(=O)Nc1ccc(-c2noc(Cc3ccc(C#N)cc3F)n2)cc1]

A solution of TBAF (1M in THF, 1.4 equiv.) was added dropwise to a solution of tert-butyl (E)-(4-(N-(2-(4-cyano-2-fluorophenyl)acetyl)-N′-hydroxycarbamimidoyl)phenyl)carbamate (255 mg, 0.62 mmol, 1 equiv.) in dry THF (6 mL), and the reaction mixture was stirred at r.t. for 2 h. The mixture was diluted with ethyl acetate, washed with water, NaHCO₃, brine, dried over MgSO₄, filtered and concentrated under reduced pressure. The residue was purified by FCC (hexane/EtOAc 9:1 to 7:3) affording the product as a white solid (142 mg, 0.36 mmol, 58% yield).

Step D

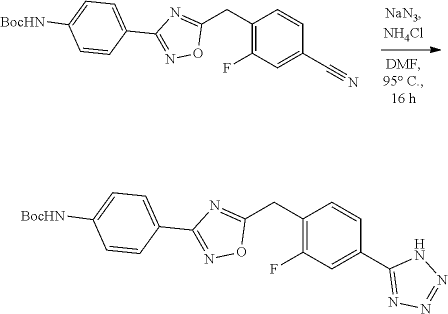 [SMILES: CC(C)(C)OC(=O)Nc1ccc(-c2noc(Cc3ccc(C#N)cc3F)n2)cc1 ; CC(C)(C)OC(=O)Nc1ccc(-c2noc(Cc3ccc(-c4nnn[nH]4)cc3F)n2)cc1]

A mixture of tert-butyl (4-(5-(4-cyano-2-fluorobenzyl)-1,2,4-oxadiazol-3-yl)phenyl)carbamate ((142 mg, 0.36 mmol, 1 equiv.), sodium azide (2 equiv.) and ammonium chloride (2 equiv.) in 2 mL DMF was stirred at 95° C. overnight. The reaction mixture was diluted with water and acidified by addition of acetic acid (70 μL). The mixture was extracted with EtOAc (2×). The organic layers were combined, washed with brine, dried (MgSO₄), filtered and concentrated under reduced pressure. The residual yellow oil obtained was used directly in the next step.

Step E

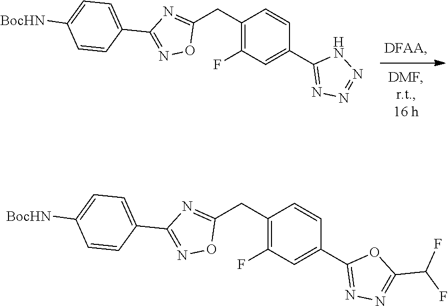 [SMILES: CC(C)(C)OC(=O)Nc1ccc(-c2noc(Cc3ccc(-c4nnn[nH]4)cc3F)n2)cc1 ; CC(C)(C)OC(=O)Nc1ccc(-c2noc(Cc3ccc(-c4nnc(C(F)F)o4)cc3F)n2)cc1]

tert-butyl (4-(5-(2-fluoro-4-(1H-tetrazol-5-yl)benzyl)-1,2,4-oxadiazol-3-yl)phenyl)carbamate (157 mg, 0.36 mmol, 1 equiv.) was dissolved in 2 mL DMF under argon. Difluoroacetic anhydride (3 equiv.) was added, the flask was sealed and the RM was stirred at r.t. overnight. The mixture was diluted with water (precipitation occurred) and extracted with EtOAc. The organic layers were washed with NaHCO₃, brine, dried (MgSO₄), filtered and concentrated under reduced pressure affording the product as a yellow oil which was used directly in the next step (158 mg, 0.32 mmol, 90% yield).

Step F

tert-butyl (4-(5-(4-(5-(difluoromethyl)-1,3,4-oxadiazol-2-yl)-2-fluorobenzyl)-1,2,4-

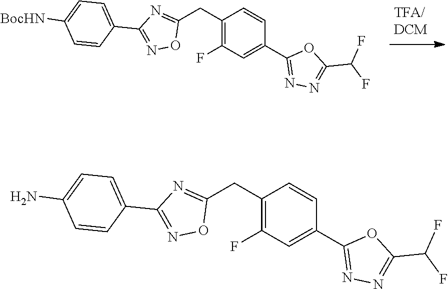 [SMILES: CC(C)(C)OC(=O)Nc1ccc(-c2noc(Cc3ccc(-c4nnc(C(F)F)o4)cc3F)n2)cc1 ; Nc1ccc(-c2noc(Cc3ccc(-c4nnc(C(F)F)o4)cc3F)n2)cc1]

oxadiazol-3-yl)phenyl)carbamate (158 mg, 0.32 mmol, 1 equiv.) was dissolved in 2 mL DCM and TFA (10 equiv.) was added. The reaction mixture was stirred at r.t. overnight. The mixture was then concentrated under reduced pressure and coevaporated with acetonitrile twice, to remove excess of TFA. The residue was dissolved in a mixture of water and sat. aq. NaHCO₃ and extracted with DCM. Volatiles was removed under reduced pressure and the resulting residue was purified by flash chromatography (hexane/EtOAc 85:15 to 1:1) affording the product as a beige solid (69 mg, 0.17 mmol, 54% yield).

Step G

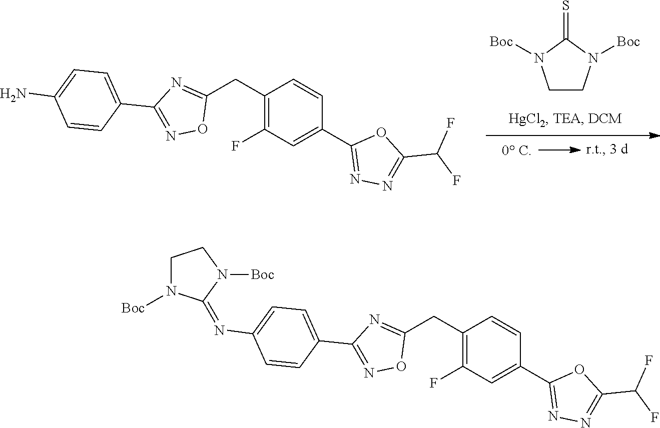 [SMILES: Nc1ccc(-c2noc(Cc3ccc(-c4nnc(C(F)F)o4)cc3F)n2)cc1 ; CC(C)(C)OC(=O)N1CCN(C(=O)OC(C)(C)C)C1=S ; CC(C)(C)OC(=O)N1CCN(C(=O)OC(C)(C)C)C1=Nc1ccc(-c2noc(Cc3ccc(-c4nnc(C(F)F)o4)cc3F)n2)cc1]

HgCl₂ (1.1 equiv.) was added to a solution of 4-(5-(4-(5-(difluoromethyl)-1,3,4-oxadiazol-2-yl)-2-fluorobenzyl)-1,2,4-oxadiazol-3-yl)aniline (69 mg, 0.17 mmol, 1 equiv.), N,N′-di(tertbutoxycarbonyl)imidazolidine-2-thione (1 equiv.) and TEA (3.1 equiv.) in 2 mL DCM at 0° C. The resulting mixture was stirred at 0° C. for 1 h and at r.t. for 3 days. The mixture was diluted with water and extracted with DCM. Organic layers were combined and washed with sat. aq. NaHCO₃ and brine, dried (MgSO₄), filtered, and concentrated under reduced pressure. The resulting beige solid was used in the next step without further purification.

Step H

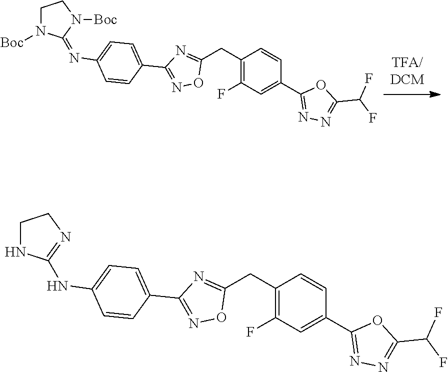 [SMILES: CC(C)(C)OC(=O)N1CCN(C(=O)OC(C)(C)C)C1=Nc1ccc(-c2noc(Cc3ccc(-c4nnc(C(F)F)o4)cc3F)n2)cc1 ; Fc1cc(-c2nnc(C(F)F)o2)ccc1Cc1nc(-c2ccc(NC3=NCCN3)cc2)no1]

di-tert-butyl 2-((4-(5-(4-(5-(difluoromethyl)-1,3,4-oxadiazol-2-yl)-2-fluorobenzyl)-1,2,4-oxadiazol-3-yl)phenyl)imino)imidazolidine-1,3-dicarboxylate (110 mg, 0.17 mmol, 1 equiv.) was dissolved in 2 mL DCM and TFA (40 equiv.) was added. The mixture was stirred at r.t. overnight. The reaction mixture was then concentrated under reduced pressure and coevaporated with acetonitrile. The residue was purified by prep-HPLC (FA) affording the product as a white solid (33 mg, 0.07 mmol, 41% yield over two steps). (m/z 456.16 [MH+]).

This compound was prepared following the same procedure:

| Compd. | Structure | m/z [MH+] |
|---|---|---|
| 419 | 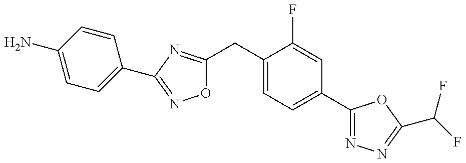 [SMILES: Nc1ccc(-c2noc(Cc3ccc(-c4nnc(C(F)F)o4)cc3F)n2)cc1] | 387.92 |

### Example 74. Synthesis of 5-(1-(1-(4-(5-(difluoromethyl)-1,3,4-oxadiazol-2-yl)phenyl)-3-(pyrrolidin-1-yl)propyl)-1H-1,2,3-triazol-4-yl)pyridin-2-amine (Compd. 323)

Step A

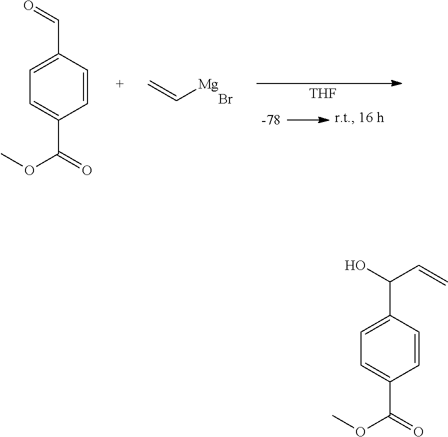

A solution of vinylmagnesium bromide (1 equiv.) in dry THF was added to a solution of methyl 4-formylbenzoate (2.4 g, 14.8 mmol, 1 equiv.) in dry THF (35 mL) at −78° C., dropwise. The resulting mixture was stirred for 1 h at −78° C. and then allowed to warm up to room temperature overnight. The reaction mixture was quenched with sat. aq. NH₄C₁ and extracted with EtOAc. The organic layers were washed with sat. aq. NaHCO₃ and brine, dried (MgSO₄), filtered and concentrated under reduced pressure affording a yellow oil which was purified by flash chromatography (hexane/EtOAc 85:15 to 75:25) (1.53 g, 7.99 mmol, 54% yield).

Step B

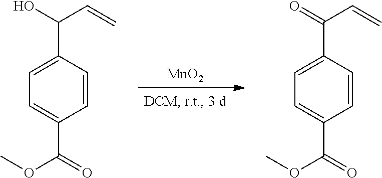

Manganese dioxide (10 equiv.) was added to a solution of methyl 4-(1-hydroxyallyl)benzoate (770 mg, 4.06 mmol, 1 equiv.) in 20 mL DCM. The reaction mixture was stirred for 3 days at r.t. The mixture was then filtered through celite, and the filtrate was concentrated under reduced pressure. The crude residue was purified by flash chromatography (hexane/EtOAc 9:1 to 4:1) to obtain the desired product as a white solid (230 mg, 1.21 mmol, 30% yield).

Step C

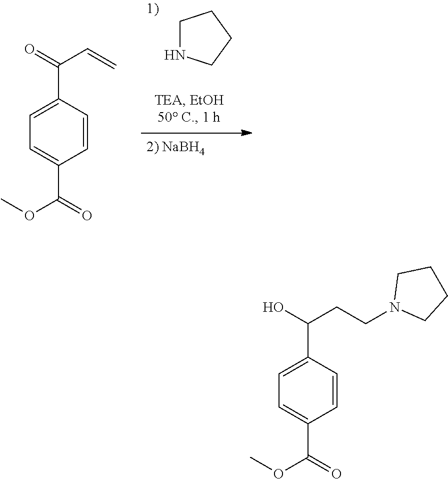 [SMILES: C=CC(=O)c1ccc(C(=O)OC)cc1 ; C1CCNC1 ; COC(=O)c1ccc(C(O)CCN2CCCC2)cc1]

Methyl 4-acryloylbenzoate (210 mg, 1.1 mmol, 1 equiv.) was dissolved in ethanol, and pyrrolidine (1 equiv.) and triethylamine (1 equiv.) were added. The mixture was stirred at 50° C. for 1 h. Then sodium borohydride (1 equiv.) was added and the reaction mixture was stirred at r.t. overnight. The reaction mixture was diluted with water and extracted with EtOAc. The organic phase was washed with brine, dried (MgSO₄), filtered and concentrated under reduced pressure. The residue was used directly in the next step.

Step D

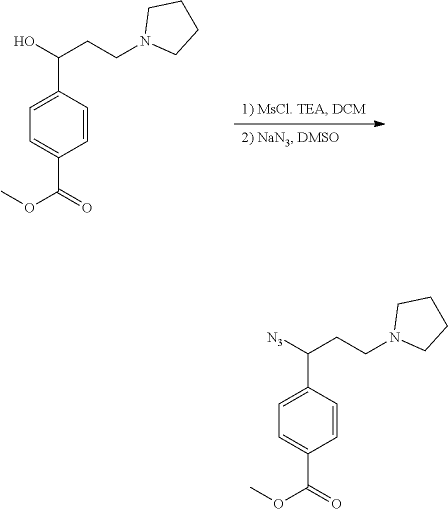 [SMILES: COC(=O)c1ccc(C(O)CCN2CCCC2)cc1 ; COC(=O)c1ccc(C(CCN2CCCC2)N=[N+]=[N-])cc1]

Triethylamine (2.5 equiv.) and mesyl chloride (2.2 equiv.) were added to a solution of methyl 4-(1-hydroxy-3-(pyrrolidin-1-yl)propyl)benzoate (306 mg, 1.16 mmol, 1 equiv.) in 8 mL DCM. The mixture was stirred at r.t. overnight. Water was added to the reaction mixture and the product was extracted with DCM. The organic layer was washed with brine, dried over MgSO₄, filtered and concentrated under reduced pressure affording a yellow solid.

The crude intermediate was dissolved in 2 mL DMSO, and sodium azide (1.5 equiv.) was added. The resulting mixture was stirred at r.t. overnight. The reaction was quenched with water and extracted with EtOAc. The organic layer was washed with brine, dried over MgSO₄, filtered and concentrated under reduced pressure. The crude product was used directly in the next step (200 mg, 0.7 mmol, 60% yield over two steps).

Step E

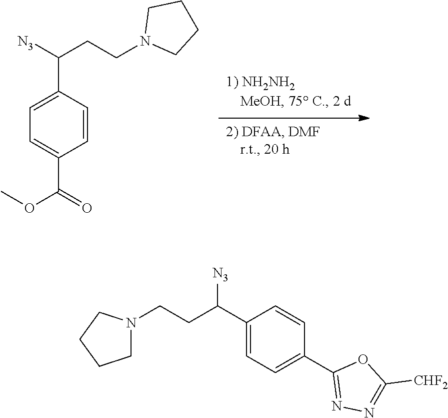 [SMILES: COC(=O)c1ccc(C(CCN2CCCC2)N=[N+]=[N-])cc1 ; [N-]=[N+]=NC(CCN1CCCC1)c1ccc(-c2nnc(C(F)F)o2)cc1]

Methyl 4-(1-azido-3-(pyrrolidin-1-yl)propyl)benzoate (200 mg, 0.7 mmol, 1 equiv.) was dissolved in 4 mL methanol and hydrazine (40 equiv.) was added. The mixture was stirred at 75° C. over 2 day. The reaction mixture was then concentrated under reduced pressure and co-evaporated with acetonitrile. The residue was dried overnight under reduced pressure, and then dissolved in 3 mL DMF, under argon.

DFAA (6 equiv.) was added, the flask was sealed and the reaction mixture was stirred at r.t. over 20 h. The mixture was diluted with water and extracted with EtOAc. The organic layers were washed with sat. aq. NaHCO₃ and brine, dried (MgSO₄), filtered and concentrated under reduced pressure. The residue was purified by flash chromatography (EtOAc/MeOH/NH₃ 100:0:0 to 95:4.5:0.5) affording the product as a yellow oil (62 mg, 0.18 mmol, 25% yield).

Step F

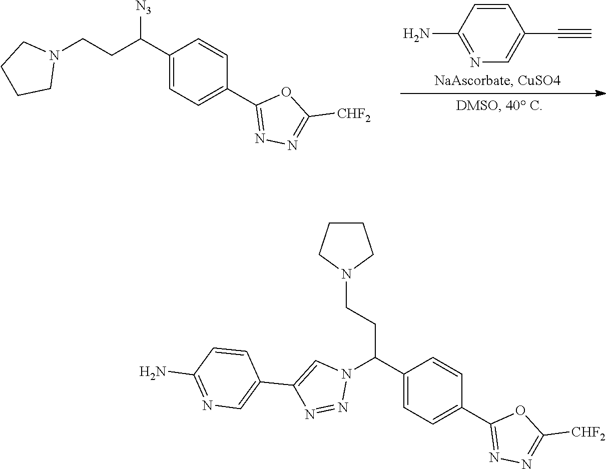

Methyl 4-(1-azido-3-(pyrrolidin-1-yl)propyl)benzoate (56 mg, 0.16 mmol, 1 equiv.) was dissolved in 1 mL DMSO. 5-ethynylpyridin-2-amine (1 equiv.) was added as a solution in 0.5 mL DMSO. CuSO₄ (0.5M in water, 0.2 equiv.) and sodium ascorbate (1M in water, 0.4 equiv.) were also added, and the resulting mixture was stirred at r.t. for 3 h. Water was added and the mixture was extracted with EtOAc. The aqueous phase was basified by addition of KOH and extracted with more EtOAc. The combined organic layers were washed with brine, dried over MgSO₄, filtered and concentrated under reduced pressure. The crude residue was purified by prep-HPLC (FA) affording the product as a white solid (17 mg, 0.036 mmol, 22% yield) (m/z 467.97 [MH+]).

The following compound was synthesized according to the same procedure

| Compd. | Structure | m/z [MH+] |
|---|---|---|
| 323 | 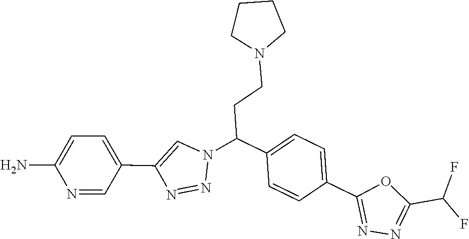 [SMILES: Nc1ccc(-c2cn(C(CCN3CCCC3)c3ccc(-c4nnc(C(F)F)o4)cc3)nn2)cn1] | 467.07 |

### Example 75. Synthesis of 4-(4-(6-aminopyridin-3-yl)-1H-1,2,3-triazol-1-yl)-4-(4-(5-(difluoromethyl)-1,3,4-oxadiazol-2-yl)phenyl)butan-1-ol (Compd. 335)

Step A

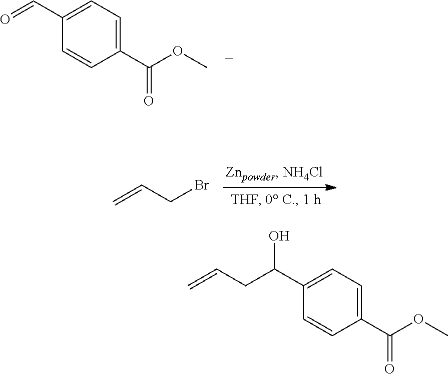

25 mL of sat. aq. NH₄C₁ were added in one portion to a stirring solution of methyl 4-formylbenzoate (2.5 g, 15.2 mmol, 1 equiv.) and allyl bromide (1 equiv.) in THF (25 mL) at 0° C. After adding zinc powder (0.24 equiv.) portionwise, the reaction mixture was stirred at the same temperature over 1 h. The reaction mixture was then poured into water (50 mL) and the product was extracted with EtOAc (3×25 mL). The extract was washed with water, sat. aq. NaHCO₃ and brine, dried over Na₂SO₄ and concentrated. The crude product (2.3 g, 11.1 mmol, 73% yield) was used in the next step without any further purification.

Step B

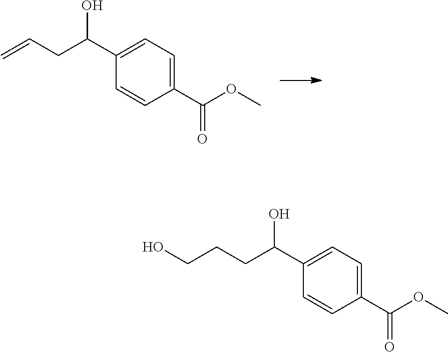 [SMILES: C=CCC(O)c1ccc(C(=O)OC)cc1 ; COC(=O)c1ccc(C(O)CCCO)cc1]

A solution of dimethylsulfide borane (2M in THF, 1.1 equiv.) was added to a solution of methyl 4-(1-hydroxybut-3-enyl)benzoate (1.1 g, 5.3 mmol, 1 equiv.) in dry THF at −5° C. over 15 min and the resulting mixture was stirred with gradient warming to r.t. over 5 h. Sodium borate hydrate (6 equiv.) was added at 0° C. followed by water (25 mL). The resulting mixture was stirred at r.t. over 12 h.

The reaction mixture was diluted with water and the product was extracted with EtOAc. Organic layers were washed with water, sat. aq. NaHCO₃ and brine, dried over Na₂SO₄ and concentrated. The residue was purified by flash chromatography (hexane/EtOAc 0-50%) to give the product as pale yellow oil (855 mg, 3.8 mmol, 71% yield).

Step C

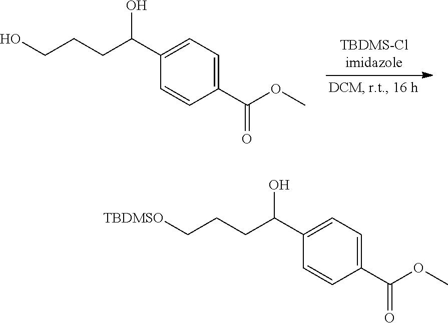 [SMILES: COC(=O)c1ccc(C(O)CCCO)cc1 ; COC(=O)c1ccc(C(O)CCCO[Si](C)(C)C(C)(C)C)cc1]

A solution of tert-butyldimethylsilyl chloride (1.1 equiv.) in dry DCM (3 mL) was added to a solution of methyl 4-(1,4-dihydroxybutyl)benzoate (855 mg, 3.8 mmol, 1 equiv.) and imidazole (1.5 equiv.) in dry DCM (12 mL) at −5° C. over 15 min. The resulting mixture was allowed to reach r.t. and it was stirred over 12 h. The reaction mixture was diluted with water and the product was extracted with EtOAc. Organic layers were washed with water, sat. aq. NaHCO₃ and brine, dried over Na₂SO₄ and concentrated. The crude residue was used in next step without additional purification.

Step D

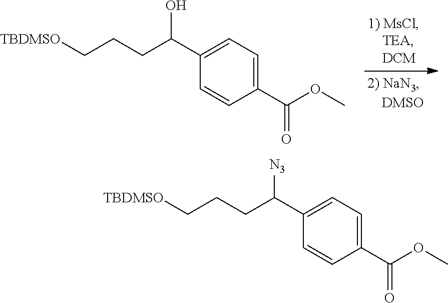

Triethylamine (3.5 equiv.) and mesyl chloride (1.5 equiv.) were added to a solution of methyl 4-(4-((tert-butyldimethylsilyl)oxy)-1-hydroxybutyl)benzoate (1.24, 3.66 mmol, 1 equiv.) in 15 mL DCM. The mixture was stirred at r.t. overnight. Water was added to the reaction mixture and the product was extracted with DCM. The organic layer was washed with brine, dried over MgSO₄, filtered and concentrated under reduced pressure.

The obtained crude intermediate was dissolved in 5 mL DMSO, and sodium azide (1.5 equiv.) was added. The resulting mixture was stirred at r.t. overnight. The reaction was quenched with water and extracted with EtOAc. The organic layer was washed with brine, dried over MgSO₄, filtered and concentrated under reduced pressure. The crude residue was purified by flash chromatography (hexane/EtOAc 0-30%) to obtain the desired product as a colourless oil (1.13 g, 3.11 mmol, 82% yield over two steps).

Step E

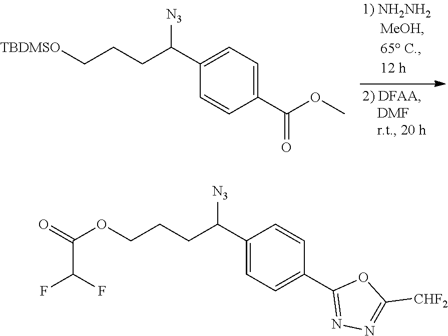 [SMILES: COC(=O)c1ccc(C(CCCO[Si](C)(C)C(C)(C)C)N=[N+]=[N-])cc1 ; [N-]=[N+]=NC(CCCOC(=O)C(F)F)c1ccc(-c2nnc(C(F)F)o2)cc1]

Methyl 4-[1-azido-4-[tert-butyl(dimethyl)silyl]oxybutyl]benzoate (185 mg, 0.51 mmol, 1 equiv.) was dissolved in 4 mL methanol and hydrazine hydrate (5 equiv.) was added. The mixture was refluxed under stirring over 12 h. The reaction mixture was then concentrated under reduced pressure and co-evaporated with acetonitrile. The residue was dried overnight under reduced pressure, and then dissolved in 2.5 mL DMF, under argon.

DFAA (4 equiv.) was added, the flask was sealed and the reaction mixture was stirred at r.t. over 12 h. The mixture was diluted with water and extracted with EtOAc. The organic layers were washed with sat. aq. NaHCO₃ and brine, dried (MgSO₄), filtered and concentrated under reduced pressure. The residue was used in the next step without any additional purification.

Step F

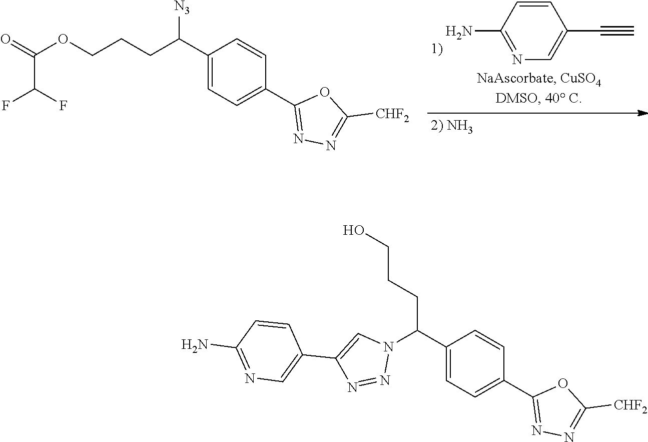

4-azido-4-(4-(5-(difluoromethyl)-1,3,4-oxadiazol-2-yl)phenyl)butyl 2,2-difluoroacetate (50 mg, 0.13 mmol, 1 equiv.) was dissolved in 0.5 mL DMSO. 5-ethynylpyridin-2-amine (1 equiv.) was added as a solution in 0.5 mL DMSO. CuSO₄ (0.5M in water, 0.2 equiv.) and sodium ascorbate (1M in water, 0.4 equiv.) were also added, and the resulting mixture was stirred at r.t. for 3 h. Full conversion to the protected intermediate was observed by LC-MS. 200 μL of 7M NH₃ (5 equiv.) in MeOH was added to the reaction mixture, which was stirred for additional 30 min. Full deprotection occurred. The reaction mixture was submitted to prep-HPLC without any workup, affording the product as a white solid (23 mg, 0.05 mmol, 39% yield) (m/z 427.95 [MH+]).

The following compound was synthesized according to the same procedure:

| Compd. | Structure | m/z [MH+] |
|---|---|---|
| 335 | 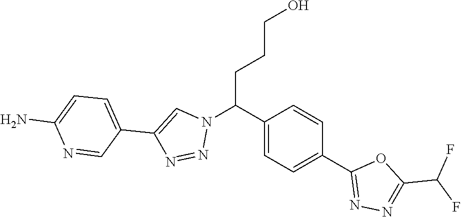 [SMILES: Nc1ccc(-c2cn(C(CCCO)c3ccc(-c4nnc(C(F)F)o4)cc3)nn2)cn1] | 427.95 |

### Example 76. Synthesis of 5-(1-((5-(5-(difluoromethyl)-1,3,4-oxadiazol-2-yl)pyridin-2-yl)methyl)-1H-imidazol-4-yl)benzo[d]oxazol-2-amine (Compd. 308)

Step A

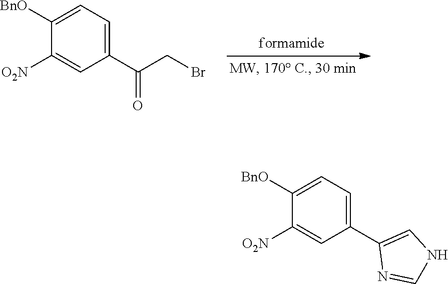 [SMILES: O=C(CBr)c1ccc(OCc2ccccc2)c([N+](=O)[O-])c1 ; O=[N+]([O-])c1cc(-c2c[nH]cn2)ccc1OCc1ccccc1]

1-(4-(benzyloxy)-3-nitrophenyl)-2-bromoethan-1-one (500 mg, 1.43 mmol, 1 equiv.) and formamide (1 equiv.) were heated by MW irradiation at 170° C. for 30 min. The mixture was then poured into 20 ml of H₂O, the pH was adjusted to 10-12 by adding 2N NaOH solution, and the resulting solid was filtered off with suction and dried, resulting in 180 mg of the desired product (0.611 mmol, 43% yield).

Step B

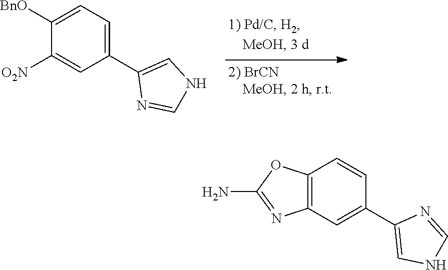 [SMILES: O=[N+]([O-])c1cc(-c2c[nH]cn2)ccc1OCc1ccccc1 ; Nc1nc2cc(-c3c[nH]cn3)ccc2o1]

4-(3-nitro-4-phenylmethoxyphenyl)-1H-imidazole (180 mg, 0.611 mmol, 1 equiv.) was dissolved in 10 mL MeOH, and 25 mg Pd/C were added. The reaction vessel was filled with hydrogen, and the mixture was stirred over weekend. The mixture was then filtered through a pad of celite, evaporated, dried under vacuum.

The crude residue was dissolved in 5 mL MeOH, and BrCN (1 equiv.) was added dropwise. The reaction mixture was stirred at r.t. for 2 h. The crude residue was purified by flash chromatography (dry load, DCM/MeOH 95:5 to 9:1) to afford 122 mg of brown solid (0.609 mmol, 99% yield).

Step C

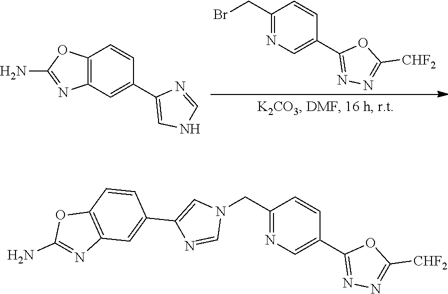 [SMILES: Nc1nc2cc(-c3c[nH]cn3)ccc2o1 ; FC(F)c1nnc(-c2ccc(CBr)nc2)o1 ; Nc1nc2cc(-c3cn(Cc4ccc(-c5nnc(C(F)F)o5)cn4)cn3)ccc2o1]

5-(1H-imidazol-4-yl)benzo[d]oxazol-2-amine (61 mg, 0.305 mmol, 1 equiv.) was dissolved in 3 mL DMF. Potassium carbonate (2 equiv.) and 2-(6-(bromomethyl)pyridin-3-yl)-5-(difluoromethyl)-1,3,4-oxadiazole (1 equiv.) were successively added. The resulting suspension was stirred at r.t. overnight. Water was added and mixture was extracted with EtOAc (4 times), dried over Na₂SO₄, filtered and concentrated under reduced pressure. The crude product was purified by prepHPLC (neutral conditions) to obtain 11 mg of the desired product (0.028, 9% yield). (m/z 409.98 [MH+]) The following compound was prepared following the same procedure:

| Compd. | Structure | m/z [MH+] |
|---|---|---|
| 309 | 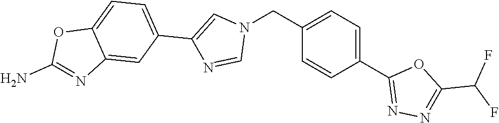 [SMILES: Nc1nc2cc(-c3cn(Cc4ccc(-c5nnc(C(F)F)o5)cc4)cn3)ccc2o1] | 408.77 |

### Example 77—Enzymatic Screening

For each test compound, 100× concentrated DMSO solutions at 8 doses were prepared and then diluted in assay buffer (25 mM Tris-HCl, pH 8, 130 mM NaCl, 0.05% Tween-20, 10% Glycerol) to obtain 5× concentrated solutions in relation to the final concentrations (typical final concentration range—6.4-200000 nM or 0.18-50000 nM, final DMSO content—1%). Then 10 μL solution of each test compound concentration were placed on a 96-well plate in triplicate and 15 μL of 3.33× concentrated enzyme solution in the assay buffer containing 3.33× concentrated BSA (final BSA concentration—2 mg/mL for HDAC4, HDAC5 and HDAC9 or 1 mg/mL for other isoforms) and in the case of HDAC6-3.33× concentrated TCEP (final TCEP concentration—200 μM) were added to each well. After a period of preincubation (incubation times and temperatures vary for different isoforms and are shown in table 1) 25 μL of solution containing the substrate were added. As substrate, FLUOR DE LYS® deacetylase substrate (Enzo Life Sciences, cat: BML-KI104, FdL), FLUOR DE LYS®-Green substrate (Enzo Life Sciences, cat: BML-KI572, FdL_G) or Boc-Lys(Tfa)-AMC (Bachem, cat: 4060676.005, Tfal)—2× concentrated solution in assay buffer were used. Following a reaction period (reaction times and temperatures vary for different isoforms and are reported in Table 1), 50 μL of the development solution consisting of concentrate FLUOR DE LYS® developer I (Enzo Life Sciences, ca: BML-KI105), diluted 200 times in buffer (50 mM Tris-HCl, pH 8, 137 mM NaCl, 2.7 mM KCl, 1 mM MgCl₂) plus 2 μM TSA was added and, after 25 minutes at room temperature in the dark, using the Victor 1420 Multilabel Counter Perkin Elmer Wallac instrument, the fluorescence reading was carried out.

TABLE 1
Operational details for the enzymatic test of each individual isoform

| | | | | | | | Reading method |
|---|---|---|---|---|---|---|---|
| Enzyme | | | | | | | λ ex/λ em |
| Isoform | Source | Conc. | | Substrate | Preincubation | Reaction | (0.1 s) |
| HDAC1 | BPS cat | 700 | pM | 25 μM | 120 minutes | 30 minutes | 485/535 nm |
| | 50051 | | | FdL_G | at 25° C. | at 25° C. | |
| HDAC2 | BPS cat | 3 | nM | 150 μM | 30 minutes | 30 minutes | 355/460 nm |
| | 50002 | | | FdL | at 30° C. | at 30° C. | |
| HDAC3 | BPS cat | 460 | pM | 25 μM | 30 minutes | 30 minutes | 485/535 nm |
| | 50003 | | | FdL_G | at 30° C. | at 30° C. | |
| HDAC4 | BPS cat | 53 | pM | 20 μM | 120 minutes | 80 minutes | 355/460 nm |
| | 50004 | | | Tfal | at 25° C. | at 25° C. | |
| HDAC5 | BPS cat | 700 | pM | 20 MM | 30 minutes | 60 minutes | 355/460 nm |
| | 50005 | | | Tfal | at 30° C. | at 30° C. | |
| HDAC6 | BPS cat | 250 | pM | 25 μM | 30 minutes | 30 minutes | 485/535 nm |
| | 50006 | | | FdL_G | at 25° C. | at 25° C. | |
| HDAC7 | BPS cat | 130 | pM | 20 μM | 60 minutes | 60 minutes | 355/460 nm |
| | 50007 | | | Tfal | at 25° C. | at 25° C. | |
| HDAC8 | BPS cat | 8.5 | nM | 25 μM | 55 minutes | 30 minutes | 485/535 nm |
| | 50008 | | | FdL_G | at RT | at 30° C. | |

Enzymatic activity on recombinant human HDAC6 and HDAC1 was evaluated (Table 2) for each synthesized compound. All compounds tested resulted virtually inactive (IC50 > 30 μM) on HDAC1. A limited number of compounds were also screened on all other isoforms in order to obtain the full profile (Table 3).

TABLE 2
Enzyme Inhibitory Activity Assay on
HDAC6 and on HDAC1 (IC50 in nM unit).

| | HDAC6 | HDAC1 | | HDAC6 | HDAC1 |
|---|---|---|---|---|---|
| | IC50 | IC50 | | IC50 | IC50 |
| EXAMPLE | (nM) | (nM) | EXAMPLE | (nM) | (nM) |
| 1 | 7 | >200000 | 216 | 151 | >200000 |
| 2 | 10 | >200000 | 217 | 154 | >200000 |
| 3 | 10 | >200000 | 218 | 156 | >200000 |
| 4 | 12 | >200000 | 219 | 156 | >200000 |
| 5 | 12 | >200000 | 220 | 157 | >200000 |
| 6 | 13 | >200000 | 221 | 157 | >200000 |
| 7 | 13 | >200000 | 222 | 160 | >200000 |
| 8 | 14 | >200000 | 223 | 161 | >200000 |
| 9 | 15 | >200000 | 224 | 162 | >200000 |
| 10 | 16 | >200000 | 225 | 169 | >200000 |
| 11 | 19 | >200000 | 226 | 169 | >200000 |
| 12 | 20 | >200000 | 227 | 172 | >200000 |
| 13 | 23 | >200000 | 228 | 178 | >200000 |
| 14 | 25 | >200000 | 229 | 179 | >200000 |
| 15 | 26 | >200000 | 230 | 187 | >200000 |
| 16 | 29 | >200000 | 231 | 197 | >200000 |
| 17 | 8 | >200000 | 232 | 198 | >200000 |
| 18 | 8 | >200000 | 233 | 200 | >200000 |
| 19 | 11 | >200000 | 234 | 202 | >200000 |
| 20 | 11 | >200000 | 235 | 202 | >200000 |
| 21 | 17 | >200000 | 236 | 206 | >200000 |
| 22 | 17 | >200000 | 237 | 210 | >200000 |
| 23 | 13 | 30320 | 238 | 211 | >200000 |
| 24 | 7 | >200000 | 239 | 215 | >200000 |
| 25 | 7 | >200000 | 240 | 215 | >200000 |
| 26 | 8 | >200000 | 241 | 216 | >200000 |
| 27 | 9 | >200000 | 242 | 252 | >200000 |
| 28 | 13 | >200000 | 243 | 234 | >200000 |
| 29 | 10 | >200000 | 244 | 240 | >200000 |
| 30 | 10 | >200000 | 245 | 264 | >200000 |
| 31 | 14 | >200000 | 246 | 266 | >200000 |
| 32 | 14 | >200000 | 247 | 272 | >200000 |
| 33 | 14 | >200000 | 248 | 275 | >200000 |
| 34 | 15 | >200000 | 249 | 277 | >200000 |
| 35 | 16 | >200000 | 250 | 293 | >200000 |
| 36 | 16 | >200000 | 251 | 298 | >200000 |
| 37 | 17 | >200000 | 252 | 27 | >200000 |
| 38 | 18 | >200000 | 253 | 352 | >200000 |
| 39 | 18 | >200000 | 254 | 361 | >200000 |
| 40 | 19 | >200000 | 255 | 377 | >200000 |
| 41 | 19 | 122900 | 256 | 393 | >200000 |
| 42 | 19 | >200000 | 257 | 395 | >200000 |
| 43 | 20 | 74921 | 258 | 410 | >200000 |
| 44 | 20 | 163267 | 259 | 439 | >200000 |
| 45 | 20 | >200000 | 260 | 480 | >200000 |
| 46 | 20 | >200000 | 261 | 181 | >200000 |
| 47 | 21 | >30000 | 262 | 488 | >200000 |
| 48 | 21 | >30000 | 263 | 489 | n.a. |
| 49 | 21 | >200000 | 264 | 19 | >200000 |
| 50 | 22 | >200000 | 265 | 26 | >200000 |
| 51 | 22 | >200000 | 266 | 35 | >200000 |
| 52 | 23 | >200000 | 267 | 85 | >200000 |
| 53 | 23 | >200000 | 268 | 254 | >200000 |
| 54 | 23 | >200000 | 269 | 15 | >200000 |
| 55 | 24 | >200000 | 270 | 18 | >200000 |
| 56 | 25 | >200000 | 271 | 43 | >200000 |
| 57 | 25 | >200000 | 272 | 407 | >200000 |
| 58 | 25 | >200000 | 273 | 22 | >200000 |
| 59 | 24 | >200000 | 274 | 11 | >200000 |
| 60 | 26 | 32870 | 275 | 373 | >200000 |
| 61 | 27 | >200000 | 276 | 9 | 164800 |
| 62 | 27 | >200000 | 277 | 72 | 165600 |
| 63 | 28 | >200000 | 278 | 186 | n.a. |
| 64 | 28 | >200000 | 279 | 286 | >200000 |
| 65 | 28 | >200000 | 280 | 89 | >200000 |
| 66 | 28 | >200000 | 281 | 200 | >200000 |
| 67 | 29 | >200000 | 282 | 282 | >200000 |
| 68 | 33 | >200000 | 283 | 225 | >200000 |
| 69 | 30 | >200000 | 284 | 323 | >200000 |
| 70 | 34 | >200000 | 285 | 491 | >200000 |
| 71 | 30 | >200000 | 286 | 99 | >200000 |
| 72 | 30 | >200000 | 287 | 446 | >200000 |
| 73 | 31 | >200000 | 288 | 355 | >200000 |
| 74 | 31 | >200000 | 289 | 203 | >200000 |
| 75 | 32 | >200000 | 290 | 68 | >200000 |
| 76 | 32 | >200000 | 291 | 129 | >200000 |
| 77 | 35 | >200000 | 292 | 30 | >200000 |
| 78 | 32 | >200000 | 293 | 31 | >200000 |
| 79 | 33 | >200000 | 294 | 78 | >200000 |
| 80 | 33 | >200000 | 295 | 32 | >200000 |
| 81 | 33 | >200000 | 296 | 34 | >200000 |
| 82 | 33 | >200000 | 297 | 37 | 64999 |
| 83 | 33 | >200000 | 298 | 151 | >200000 |
| 84 | 33 | >200000 | 299 | 110 | >200000 |
| 85 | 33 | >200000 | 300 | 67 | >200000 |
| 86 | 34 | >200000 | 301 | 64 | >200000 |
| 87 | 34 | >200000 | 302 | 49 | >999999 |
| 88 | 34 | >200000 | 303 | 62 | >999999 |
| 89 | 34 | >200000 | 304 | 35 | >999999 |
| 90 | 34 | >200000 | 305 | 23 | >999999 |
| 91 | 35 | >200000 | 306 | 32 | >999999 |
| 92 | 35 | >200000 | 307 | 11 | >999999 |
| 93 | 36 | >200000 | 308 | 15 | >999999 |
| 94 | 36 | >200000 | 309 | 69 | >999999 |
| 95 | 37 | >200000 | 310 | 138 | >999999 |
| 96 | 37 | >200000 | 311 | 25 | >999999 |
| 97 | 38 | >200000 | 312 | 41 | >999999 |
| 98 | 38 | >200000 | 313 | 308 | >999999 |
| 99 | 38 | >200000 | 314 | 22 | >999999 |
| 100 | 39 | >200000 | 315 | 668 | >999999 |
| 101 | 39 | >200000 | 316 | 56 | >999999 |
| 102 | 39 | >200000 | 317 | 34 | >999999 |
| 103 | 39 | >200000 | 318 | 40 | >999999 |
| 104 | 40 | >200000 | 319 | 87 | >999999 |
| 105 | 41 | >200000 | 320 | 28 | >999999 |
| 106 | 41 | >200000 | 321 | 98 | >999999 |
| 107 | 41 | >200000 | 322 | 17 | >999999 |
| 108 | 45 | 66560 | 323 | 49 | 47930 |
| 109 | 42 | >200000 | 324 | 18 | >999999 |
| 110 | 42 | >200000 | 325 | 26 | >999999 |
| 111 | 42 | >200000 | 326 | 219 | >999999 |
| 112 | 42 | >200000 | 327 | 40 | >999999 |
| 113 | 43 | >200000 | 328 | 14 | >999999 |
| 114 | 43 | >200000 | 329 | 27 | >999999 |
| 115 | 43 | >200000 | 330 | 465 | >999999 |
| 116 | 44 | >200000 | 331 | 52 | >999999 |
| 117 | 44 | >200000 | 332 | 67 | >999999 |
| 118 | 44 | >200000 | 333 | 321 | >999999 |
| 119 | 45 | >200000 | 334 | 65 | >999999 |
| 120 | 45 | >200000 | 335 | 62 | >999999 |
| 121 | 45 | >200000 | 336 | 57 | >999999 |
| 122 | 46 | >200000 | 337 | 45 | >999999 |
| 123 | 46 | >200000 | 338 | 101 | >999999 |
| 124 | 47 | >200000 | 339 | 24 | >999999 |
| 125 | 48 | >200000 | 340 | 41 | >999999 |
| 126 | 49 | >200000 | 341 | 135 | >999999 |
| 127 | 49 | >200000 | 342 | 32 | >999999 |
| 128 | 49 | >200000 | 343 | 31 | >999999 |
| 129 | 50 | >200000 | 344 | 47 | 84350 |
| 130 | 50 | >200000 | 345 | 24 | 168200 |
| 131 | 51 | >200000 | 346 | 26 | >999999 |
| 132 | 51 | >200000 | 347 | 27 | >999999 |
| 133 | 51 | >200000 | 348 | 26 | >999999 |
| 134 | 52 | >200000 | 349 | 48 | 177500 |
| 135 | 53 | >200000 | 350 | 22 | >999999 |
| 136 | 53 | >200000 | 351 | 21 | >999999 |
| 137 | 53 | >200000 | 352 | 60 | >999999 |
| 138 | 54 | >200000 | 353 | 42 | >999999 |
| 139 | 55 | >200000 | 354 | 25 | 182900 |
| 140 | 55 | >200000 | 355 | 62 | >999999 |
| 141 | 56 | >200000 | 356 | 43 | >999999 |
| 142 | 59 | >200000 | 357 | 12 | >999999 |
| 143 | 60 | >200000 | 358 | 52 | 123800 |
| 144 | 60 | >200000 | 359 | 11 | >999999 |
| 145 | 61 | >200000 | 360 | 18 | >999999 |
| 146 | 62 | >200000 | 361 | 9 | >999999 |
| 147 | 63 | >200000 | 362 | 18 | >999999 |
| 148 | 63 | >200000 | 363 | 29 | >999999 |
| 149 | 64 | >200000 | 364 | 37 | 570399.5 |
| 150 | 64 | >200000 | 365 | 106 | >999999 |
| 151 | 65 | >200000 | 366 | 297 | >999999 |
| 152 | 65 | >200000 | 367 | 26 | 167700 |
| 153 | 66 | >200000 | 368 | 17 | >999999 |
| 154 | 67 | >200000 | 369 | 17 | >999999 |
| 155 | 69 | >200000 | 370 | 23 | >999999 |
| 156 | 70 | >200000 | 371 | 25 | >999999 |
| 157 | 70 | >200000 | 372 | 19 | >999999 |
| 158 | 72 | >200000 | 373 | 26 | >999999 |
| 159 | 74 | >200000 | 374 | 101 | >999999 |
| 160 | 74 | >200000 | 375 | 88 | >999999 |
| 161 | 75 | >200000 | 376 | 9 | >999999 |
| 162 | 76 | 45109 | 377 | 370 | >999999 |
| 163 | 77 | >200000 | 378 | 17 | 199200 |
| 164 | 77 | >200000 | 379 | 17 | >999999 |
| 165 | 78 | >200000 | 380 | 19 | >999999 |
| 166 | 78 | >200000 | 381 | 28 | >999999 |
| 167 | 79 | >200000 | 382 | 18 | >999999 |
| 168 | 79 | >200000 | 383 | 13 | >999999 |
| 169 | 81 | >200000 | 408 | 80 | >999999 |
| 170 | 81 | >200000 | 419 | 64 | >999999 |
| 171 | 82 | >200000 | 420 | 12 | >999999 |
| 172 | 86 | >200000 | 384 | 63 | 999999 |
| 173 | 86 | >200000 | 385 | 30 | 999999 |
| 174 | 87 | >200000 | 386 | 40 | 999999 |
| 175 | 88 | >200000 | 387 | 12 | 999999 |
| 176 | 88 | >200000 | 388 | 125 | 999999 |
| 177 | 89 | >200000 | 389 | 192 | 999999 |
| 178 | 89 | >200000 | 390 | 116 | 999999 |
| 179 | 90 | >200000 | 391 | 217 | 999999 |
| 180 | 92 | >200000 | 392 | 102 | 999999 |
| 181 | 92 | >200000 | 393 | 497 | 999999 |
| 182 | 92 | >200000 | 394 | 264 | 999999 |
| 183 | 93 | >200000 | 395 | 857 | 999999 |
| 184 | 96 | >200000 | 396 | 60 | 999999 |
| 185 | 96 | >200000 | 397 | 57 | 999999 |
| 186 | 98 | >200000 | 398 | 49 | n.a. |
| 187 | 41 | >200000 | 399 | 247 | 999999 |
| 188 | 99 | >200000 | 400 | 32 | n.a. |
| 189 | 99 | >200000 | 401 | 144 | 999999 |
| 190 | 99 | >200000 | 403 | 117 | 999999 |
| 191 | 102 | >200000 | 404 | 178 | 999999 |
| 192 | 102 | >200000 | 405 | 59 | 999999 |
| 193 | 104 | >200000 | 406 | 85 | 999999 |
| 194 | 104 | >200000 | 407 | 136 | 999999 |
| 195 | 104 | >200000 | 409 | 420 | 999999 |
| 196 | 107 | >200000 | 410 | 348 | 999999 |
| 197 | 107 | >200000 | 413 | 515 | 999999 |
| 198 | 109 | >200000 | 414 | 461 | 999999 |
| 199 | 109 | >200000 | 415 | 49 | 999999 |
| 200 | 113 | >200000 | 416 | 46 | 999999 |
| 201 | 115 | >200000 | 417 | 44 | n.a. |
| 202 | 116 | >200000 | 418 | 27 | n.a. |
| 203 | 118 | >200000 | 422 | 249 | 999999 |
| 204 | 122 | n.a. | 423 | 99 | 999999 |
| 205 | 122 | >200000 | 424 | 76 | n.a. |
| 206 | 127 | >200000 | 425 | 545 | n.a. |
| 207 | 129 | 999000 | 426 | 345 | 999999 |
| 208 | 132 | >200000 | 427 | 72 | n.a. |
| 209 | 133 | >200000 | 428 | 99 | n.a. |
| 210 | 138 | >200000 | 429 | 146 | n.a. |
| 211 | 147 | >200000 | 431 | 352 | n.a. |
| 212 | 147 | >200000 | 433 | 23 | n.a. |
| 213 | 150 | >200000 | 434 | 59 | n.a. |
| 214 | 151 | >200000 | 504 | 100 | n.a. |
| 215 | 151 | >200000 | | | |

n.a. = not available
Preferred compounds of the present invention show HDAC6 IC₅₀ values below 500 nM, most of them show IC₅₀ values below 20 nM. All compounds are inactive on HDAC1.

TABLE 3
inhibition profile on all HDACs for some preferred
compounds according to the invention (IC₅₀ nM)

| Compd. | HDAC2 | HDAC3 | HDAC4 | HDAC5 | HDAC7 | HDAC8 | HDAC9 | HDAC10 | HDAC11 |
|---|---|---|---|---|---|---|---|---|---|
| 1 | >30000 | >30000 | >30000 | >30000 | >30000 | >200000 | >30000 | >30000 | >30000 |
| 2 | >30000 | >30000 | >30000 | >30000 | >30000 | >200000 | >30000 | >30000 | >30000 |
| 3 | >30000 | >30000 | >30000 | >30000 | >30000 | >200000 | 22467 | >30000 | >30000 |
| 4 | >30000 | >30000 | >30000 | >30000 | >30000 | >200000 | >30000 | >30000 | >30000 |
| 5 | >30000 | >30000 | >30000 | >30000 | >30000 | >200000 | >30000 | >30000 | >30000 |
| 6 | >30000 | >30000 | >30000 | >30000 | >30000 | >200000 | >30000 | >30000 | >30000 |
| 7 | >30000 | >30000 | >30000 | >30000 | >30000 | >200000 | >30000 | >30000 | >30000 |
| 8 | >30000 | >30000 | >30000 | >30000 | >30000 | >200000 | >30000 | >30000 | >30000 |
| 9 | >30000 | >30000 | 5012 | 1507 | 3079 | 1405 | 971 | >30000 | >30000 |
| 10 | >30000 | >30000 | >30000 | >30000 | >30000 | 29507 | >30000 | >30000 | >30000 |
| 11 | >30000 | >30000 | >30000 | >30000 | >30000 | >200000 | >30000 | >30000 | >30000 |
| 12 | >30000 | >30000 | >30000 | >30000 | >30000 | >200000 | >30000 | >30000 | >30000 |
| 13 | >30000 | >30000 | >30000 | >30000 | >30000 | 197100 | >30000 | >30000 | >30000 |
| 14 | >30000 | >30000 | >30000 | >30000 | >30000 | >200000 | >30000 | >30000 | >30000 |
| 15 | >30000 | >30000 | >30000 | >30000 | >30000 | >200000 | >30000 | >30000 | >30000 |
| 16 | >30000 | >30000 | >30000 | >30000 | >30000 | >200000 | >30000 | >30000 | >30000 |
| 17 | >30000 | >30000 | >30000 | >30000 | >30000 | >200000 | >30000 | >30000 | >30000 |
| 18 | >30000 | >30000 | >30000 | >30000 | >30000 | >200000 | >30000 | >30000 | >30000 |
| 19 | >30000 | >30000 | >30000 | >30000 | >30000 | 572250 | >30000 | >30000 | >30000 |
| 20 | >30000 | >30000 | >30000 | >30000 | >30000 | 577500 | >30000 | >30000 | >30000 |
| 21 | >30000 | >30000 | >30000 | >30000 | >30000 | >200000 | >30000 | >30000 | >30000 |
| 22 | >30000 | >30000 | >30000 | >30000 | >30000 | >200000 | >30000 | >30000 | >30000 |
| 23 | >30000 | 26524 | >30000 | >30000 | >30000 | 565 | >30000 | >30000 | >30000 |

### Example 78—Cytotoxicity

Cytotoxicity activity was evaluated on B697 promyelocytic cell line for most of the synthesized compounds, which showed a very safe profile, as they are nearly completely inactive.

Cells were seeded in plate (2×10⁴ cells per well). The serial dilutions of test compounds were prepared in DMSO and then diluted 1000× in culture medium (RPMI Medium 1640 supplemented with 10% FBS). Then 100 μl of compounds solutions were transferred to 100 μl of cells suspensions (final concentration ranges 0.13 nM-10000 nM, final DMSO content—0.05%) and incubated 48 hours. The molecules cytotoxic activity was evaluated using CellTiter 96® Aqueous One Solution Cell Proliferation Assay (Promega), which measures the mitochondria function, following the manufacturer's instructions.

IC₅₀ values are shown in Table 4.

TABLE 4
Cell Cytotoxicity on 697 B-precursor acute lymphoblastic leukemia
cell line (IC₅₀ nM) for some preferred compounds.

| | Citotoxicity | | Citotoxicity |
|---|---|---|---|
| Compd. | (697 Cell line) nM | Compd. | (697 Cell line) nM |
| 1 | >200000 | 50 | >200000 |
| 2 | >200000 | 52 | >200000 |
| 3 | >200000 | 56 | >200000 |
| 4 | >200000 | 63 | >200000 |
| 5 | >200000 | 64 | 100000 |
| 6 | >200000 | 65 | >200000 |
| 7 | >200000 | 67 | >200000 |
| 8 | >200000 | 70 | >200000 |
| 9 | >200000 | 71 | >200000 |
| 10 | >200000 | 73 | 100000 |
| 11 | >200000 | 75 | >200000 |
| 12 | >200000 | 78 | 100000 |
| 13 | >200000 | 79 | >200000 |
| 14 | >200000 | 80 | >200000 |
| 15 | >200000 | 81 | >200000 |
| 16 | >200000 | 86 | >200000 |
| 17 | >200000 | 89 | >200000 |
| 18 | >200000 | 90 | >200000 |
| 19 | >200000 | 92 | >200000 |
| 20 | >200000 | 96 | >200000 |
| 21 | >200000 | 98 | 100000 |
| 22 | >200000 | 100 | >200000 |
| 23 | >200000 | 101 | >200000 |
| 33 | >200000 | 104 | >200000 |
| 42 | 100000 | 105 | >200000 |
| 43 | >200000 | 106 | >200000 |
| 44 | 100000 | 109 | >200000 |
| 45 | >200000 | 113 | >200000 |
| 47 | >200000 | 114 | >200000 |
| 48 | >200000 | 116 | >200000 |
| 117 | >200000 | | |
| 120 | >200000 | | |
| 121 | >200000 | | |
| 122 | >200000 | | |
| 128 | >200000 | | |
| 133 | >200000 | | |
| 151 | >200000 | | |
| 218 | >200000 | | |
| 255 | >200000 | | |

### Example 79— Stability to Phase I Metabolism in Rat and Human S9 Liver Fraction

Test compounds were incubated in rat and human liver S9 fraction at 37° C. up to 90 minutes in order to evaluate their stability to Phase I metabolism by hepatic enzymes. Each test compound was incubated at μM concentration (50 μM when the samples were analysed by UV/HPLC, 1 or 2 μM when the samples were analysed by LC-MS/MS) with S9 fraction (protein content 2 mg/m L) in 100 mM phosphate buffer (pH 7.4), 3.3 mM MgCl₂ and 1.3 mM NADPH for 0, 10, 30, 60 and 90 minutes at 37° C. in a thermostated oscillating bath. The reaction was stopped placing samples on ice bath and adding acidified acetonitrile. After centrifugation (10 minutes at 14000 rpm) an aliquot of the supernatant was diluted with water, filtered with 0.45 μm regenerated cellulose syringe filters and injected in HPLC-UV or in LC-MS/MS. The percentage of the remaining amount at the various incubation times with respect to the initial amount were calculated. The intrinsic clearance was also calculated.

### Example 80—Stability in Rat and Human Plasma

In order to evaluate the stability to circulating enzymes, test compounds were incubated in human and rat plasma at 37° C. in a thermostated oscillating bath. Each test compound was incubated at μM concentration (50 μM when the samples were analysed by UV/HPLC, 1 or 2 μM when the samples were analysed by LC-MS/MS) for 0, 15, 30 minutes and 1, 2 and 4 hours. The reaction was stopped placing samples on ice bath and adding acidified acetonitrile. After centrifugation (10 minutes at 14000 rpm) an aliquot of the supernatant was diluted with water, filtered with 0.45 μm regenerated cellulose syringe filters and injected in HPLC-UV or in LC-MS/MS. The percentage of the remaining amount at the various incubation times with respect to the initial amount were calculated. The half-life in plasma was also calculated.

In vitro metabolic stability data for a limited number of compounds are summarized in table 5. Most of the molecules showed good stability. Notably, the most potent compounds are the most stable, too.

TABLE 5
In vitro enzymatic stability assay of preferred compounds (residual
percentage in S9 after 90 minutes and in plasma after 4 hours).

| | HUMAN | RAT | HUMAN S9 | RAT S9 |
|---|---|---|---|---|
| Compd. | PLASMA | PLASMA | FRACTION | FRACTION |
| 1 | 85 | 79 | 93 | 96 |
| 2 | 87 | 90 | 70 | 61 |
| 3 | 70 | 80 | 97 | 90 |
| 4 | 80 | 78 | 77 | 77 |
| 5 | 74 | 70 | 92 | 93 |
| 6 | 93 | 93 | 99 | 93 |
| 7 | 76 | 81 | 92 | 96 |
| 8 | 99 | 76 | 95 | 94 |
| 9 | 59 | 87 | 83 | 90 |
| 10 | 89 | 97 | 95 | 95 |
| 11 | 83 | 86 | 70 | 90 |
| 12 | 82 | 86 | 96 | 98 |
| 13 | 78 | 75 | 97 | 98 |
| 14 | 72 | 83 | 93 | 95 |
| 15 | 81 | 72 | 91 | 79 |
| 16 | 75 | 84 | 86 | 88 |
| 17 | 82 | 85 | 84 | 99 |
| 18 | 84 | 69 | 82 | 91 |
| 19 | 83 | 83 | 96 | 94 |
| 20 | 97 | 82 | 99 | 93 |
| 21 | 84 | 75 | 99 | 90 |
| 22 | 92 | 96 | 88 | 98 |
| 23 | 88 | 92 | 88 | 96 |
| 28 | 74 | 64 | 98 | 97 |
| 30 | 87 | 68 | 80 | 96 |
| 32 | 97 | 67 | 97 | 91 |
| 34 | 95 | 69 | 89 | 83 |
| 36 | 78 | 76 | 89 | 94 |
| 37 | 90 | 83 | 71 | 78 |
| 39 | 83 | 71 | 100 | 96 |
| 41 | 91 | 77 | 94 | 92 |
| 46 | 74 | 72 | 81 | 60 |
| 50 | 89 | 87 | 95 | 66 |
| 51 | 90 | 79 | 90 | 62 |
| 53 | 77 | 78 | 88 | 91 |
| 57 | 62 | 68 | 70 | 86 |
| 59 | 87 | 83 | 98 | 97 |
| 60 | 76 | 81 | 70 | 94 |
| 62 | 88 | 69 | 95 | 86 |
| 64 | 69 | 70 | 103 | 9 |
| 65 | 94 | 76 | 99 | 35 |
| 67 | 74 | 74 | 77 | 39 |
| 69 | 79 | 79 | 97 | 83 |
| 75 | 96 | 76 | 85 | 90 |
| 76 | 74 | 73 | 53 | 89 |
| 78 | 62 | n.a. | 94 | 78 |
| 79 | 85 | 77 | 96 | 78 |
| 80 | 97 | 88 | 94 | 46 |
| 81 | 87 | 87 | 78 | 14 |
| 84 | 80 | 60 | 85 | 62 |
| 89 | 81 | 82 | 96 | 98 |
| 90 | 80 | 88 | 83 | 13 |
| 91 | 78 | 93 | 74 | 68 |
| 93 | 74 | 79 | 72 | 59 |
| 97 | 76 | 96 | 84 | 95 |
| 99 | 86 | 93 | 52 | 18 |
| 102 | 92 | 90 | 82 | 56 |
| 104 | 92 | 87 | 86 | 78 |
| 105 | 84 | 81 | 99 | 71 |
| 106 | 86 | 78 | 78 | 69 |
| 108 | 78 | 77 | 96 | 68 |
| 109 | 89 | 78 | 82 | 87 |
| 113 | 75 | 92 | 76 | 85 |
| 116 | 82 | 75 | 92 | 71 |
| 117 | 92 | 74 | 93 | 71 |
| 122 | 88 | 84 | 78 | 10 |
| 124 | 94 | 92 | 91 | 96 |
| 128 | 96 | 83 | 30 | 13 |
| 131 | 92 | 82 | 83 | 58 |
| 133 | 88 | 69 | 98 | 78 |
| 138 | 92 | n.a. | 70 | 66 |
| 140 | 99 | 58 | 87 | 0 |
| 143 | 60 | 80 | 89 | 71 |
| 151 | 98 | 97 | 83 | 79 |
| 174 | 88 | 80 | 91 | 72 |
| 185 | 97 | 62 | 86 | 69 |
| 186 | 94 | 86 | 88 | 90 |
| 193 | 81 | 92 | 87 | 80 |
| 194 | 90 | 88 | 95 | 77 |
| 207 | 91 | 70 | 72 | 0 |
| 211 | 91 | 89 | 63 | 80 |
| 218 | 92 | 69 | 86 | 89 |
| 235 | 90 | 84 | 92 | 63 |
| 247 | 99 | 81 | 98 | 65 |
| 250 | 91 | 82 | 88 | 89 |
| 255 | 91 | 57 | 98 | 76 |
| 264 | 80 | 94 | 93 | 92 |
| 269 | 80 | 71 | 81 | 69 |
| 270 | 89 | 78 | 93 | 79 |
| 273 | 83 | 74 | 92 | 99 |
| 274 | 83 | 83 | 95 | 97 |
| 288 | 78 | 86 | 54 | 9 |

n.a. = not available

### Example 81—In Vitro α-Tubulin Acetylation in 697 Cell Line

The in vitro α-tubulin acetylation determination was evaluated on B 697 promyelocytic cell line.

The test molecules were diluted from 20 mM stock solution in DMSO with RPMI 10% FCS+0.01% DMSO medium at 20× concentration compared to the final concentration, added to the cells (15×10⁶ cells in a total volume of 30 mL in RPMI medium 10% FCS+0.01% DMSO) to obtain the final concentrations of 1000, 333, 111, 37 nM and incubated at 37° C., 5% CO₂ for 16 hours.

At the end of the incubation period, 5×10⁶ cells were taken from each sample, centrifuged for 5 minutes at 1100 rpm and washed in 0.9% NaCl at 4° C. The resulting pellet was lysed by treating at 4° C. for 30 minutes with 150 μl of Complete Lysis-M buffer containing protease inhibitors (Complete Lysis-M Roche+Complete Tablets, Mini Easypack, cat: 4719956001) and phosphatase inhibitor cocktails (PhosStop Easypack, Roche, cat: 4906837001), then centrifuged 10 minutes at 14,000 rpm (20817×g). 0.3 μg of supernatant (total protein extract) were diluted in 100 μl of 1×PBS and immobilized in Maxisorp F96 NUN-IMMUNO Plate (Nunc MaxiSorp flat-bottom, Nunc, cat: 442404) at room temperature overnight. Plates were washed twice with Wash Buffer (PBS1×+0.005% tween 20) and saturated for 1 hour at room temperature with 300 μL of 1×PBS containing 10% FCS. After washing twice with Wash Buffer, the plates were incubated for two hours at room temperature in the presence of anti-acetylated-α-tubulin antibody (Monoclonal Anti-acetylated-tubulin clone 6-11B-1, mouse ascites fluid, Sigma, cat: T6793), 100 μl/well diluted 1:1000 in 1×PBS containing 10% FCS) or with total anti-α-tubulin antibody (Monoclonal Anti-alpha-tubulin produced in mouse, Sigma, cat: T6074). Following washing of the pates 5 times with Wash Buffer the secondary antibody conjugated with the enzyme HRP (Goat anti-Mouse IgG, IgM, IgA (H+L), Thermo Fisher Scientific, cat: A10668), diluted 1:1000 in 1×PBS+10% FBS was added at the volume of 100 μl/well.

After washing 4 times, 100 μl/well of TMB substrate kit was added for 10 minutes at room temperature in the dark. The reaction was stopped by adding 50 μl of 2N H₂SO₄. The plates were read at Multiskan Spectrum spectrophotometer at a wavelength of 450 nm.

The degree of acetylation was calculated by dividing the absorbance obtained for acetylated α-tubulin by the absorbance of total tubulin.

The results of tubulin acetylation, expressed as fold increase of ratio of acetylated α-tubulin/total α-tubulin, of each sample relative to the control sample (untreated) are summarized in table 6.

TABLE 6
Tubulin acetylation in 697 cell line for a limited number
of compounds (fold increase of the ratio of acetylated
tubulin and total tubulin towards control.

| | fold increase |
|---|---|
| Compd. | @ 1 uM TubAc |
| 1 | 25 |
| 2 | 26 |
| 3 | 13 |
| 4 | 13 |
| 5 | 19 |
| 6 | 19 |
| 7 | 16 |
| 8 | 10 |
| 9 | 17 |
| 10 | 11 |
| 11 | 18 |
| 12 | 11 |
| 13 | 16 |
| 14 | 19 |
| 15 | 16 |
| 16 | 19 |
| 17 | 32 |
| 18 | 11 |
| 19 | 4 |
| 20 | 11 |
| 21 | 16 |
| 22 | 17 |
| 23 | 31 |
| 29 | 15 |
| 32 | 31 |
| 34 | 28 |
| 41 | 4 |
| 46 | 5 |
| 51 | 26 |
| 89 | 8 |
| 104 | 15 |
| 116 | 5 |
| 124 | 13 |
| 274 | 12 |

1-18. (canceled)

19. A compound of formula (I), or a pharmaceutically acceptable salt, isomer or prodrug thereof: 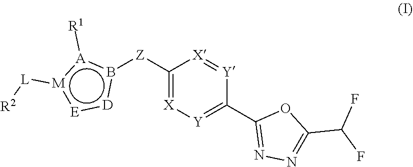
wherein:
X and X′ are dependently CH, N, CF or CCl;
Y and Y′ are independently CH, N or CF;
A is C, N, O or S;
B is C or N;
D is C, N or O;
E is C, N or O;
M is C or N;
Z is —CD₂-, —CF₂—, —CHR³—, —NH— or —S—;
R³ is H, C₁-C₄ alkyl or is selected from the following structures: 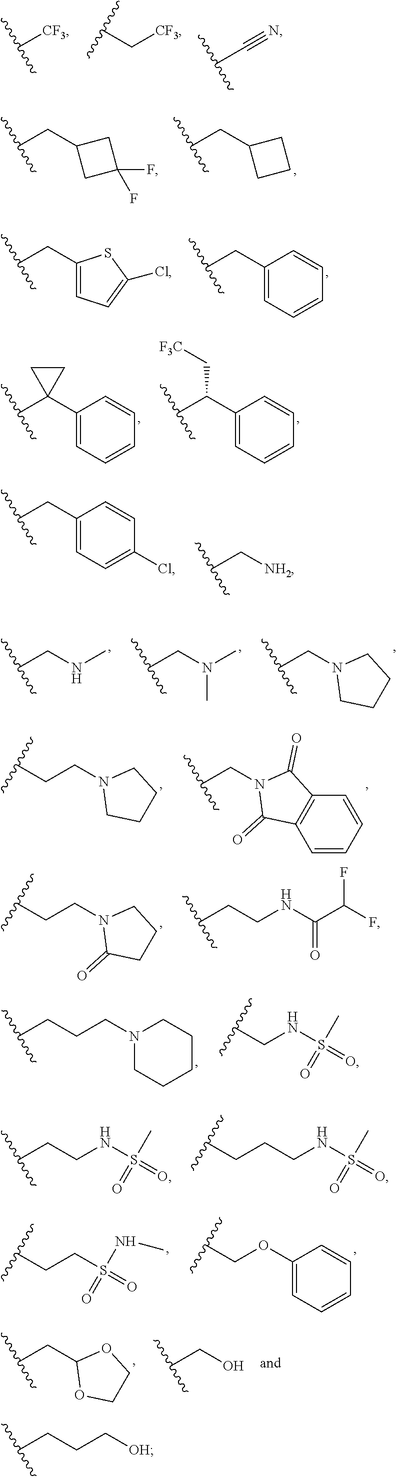
L is absent C₁-C₄ alkyl, —CHPh-, —CH₂NHCH₂—, or is selected from the following substructure: 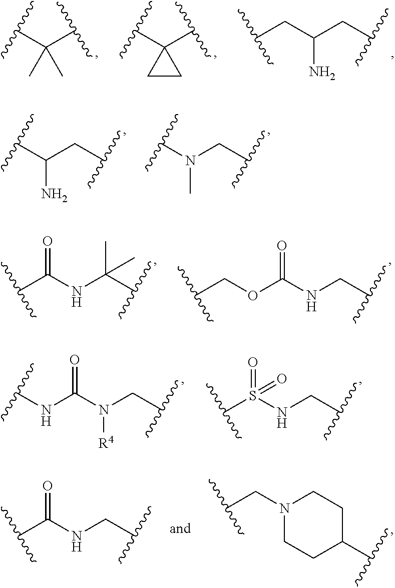
wherein R⁴ is H or C₁-C₄ alkyl;
R¹ is absent, —H, C₁-C₄ alkyl or -LR², wherein when R¹ is -LR², substitution on M is absent;
R² is selected from the group consisting of: 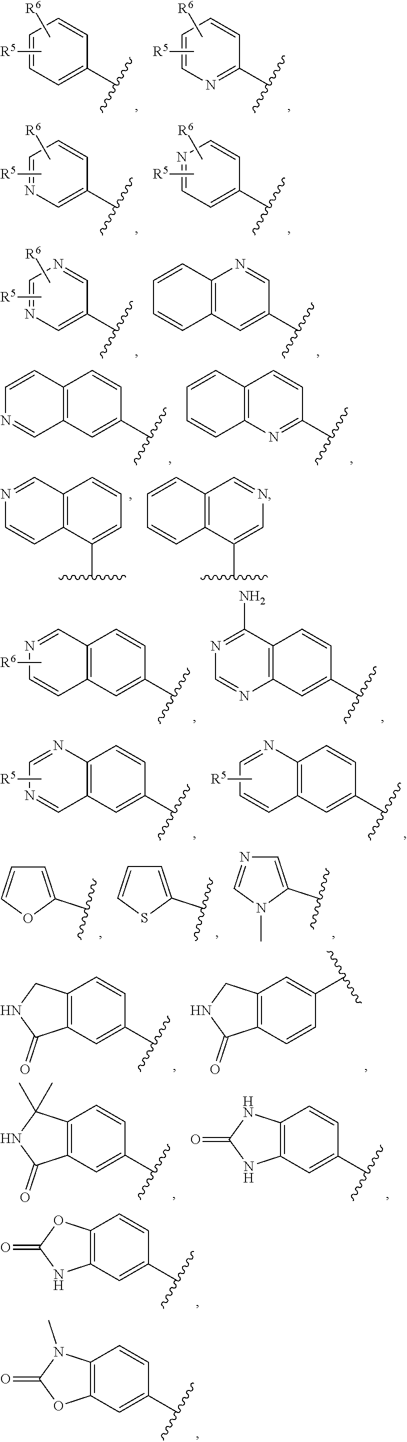 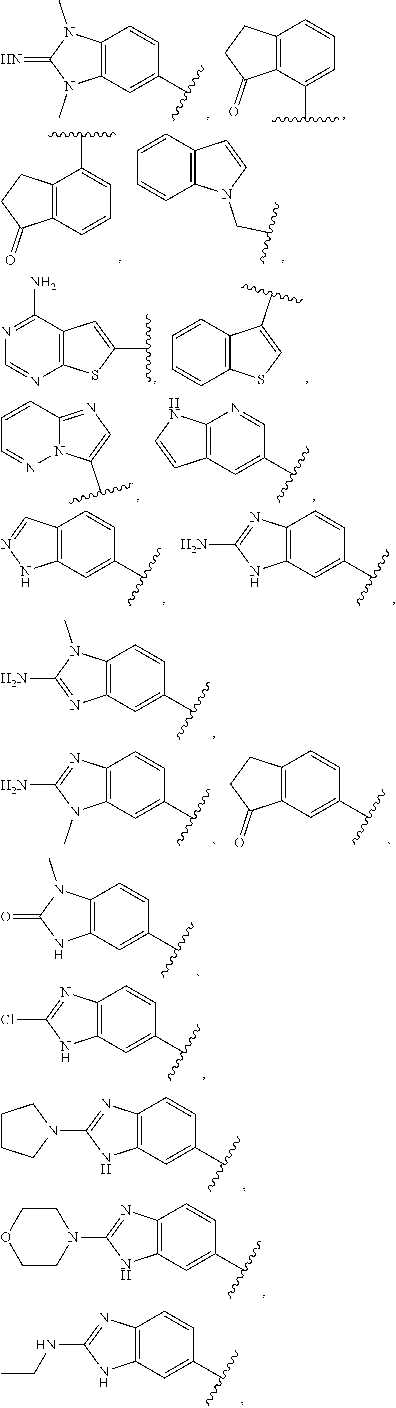 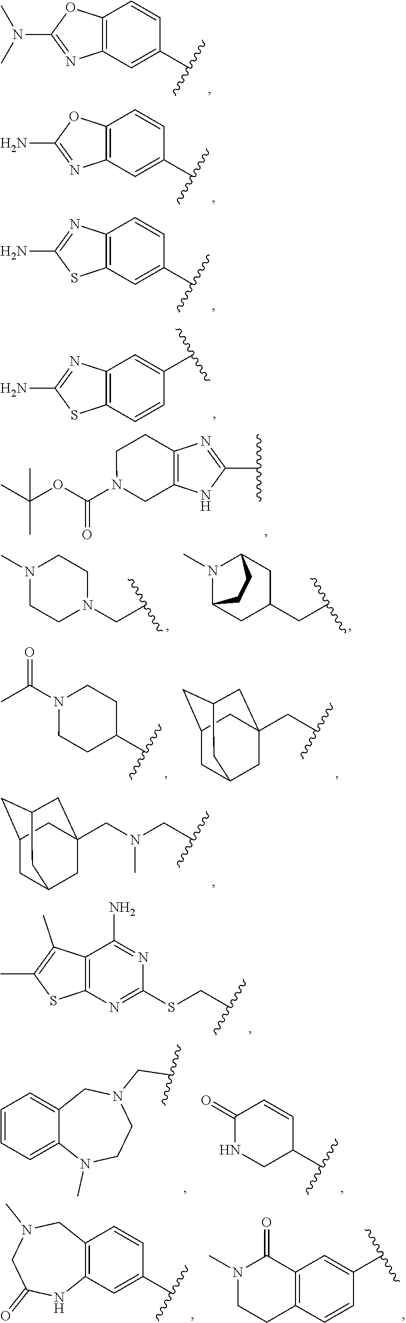 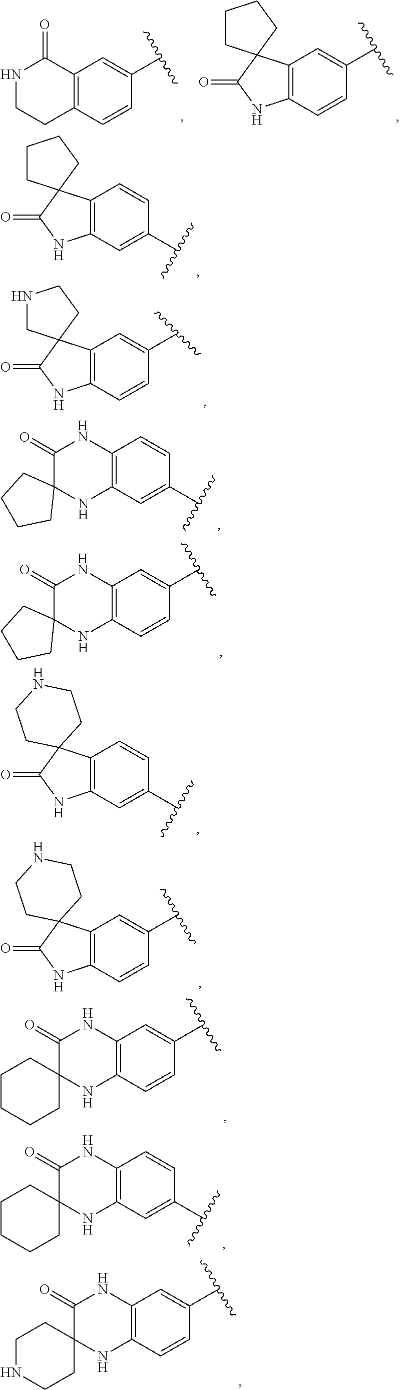 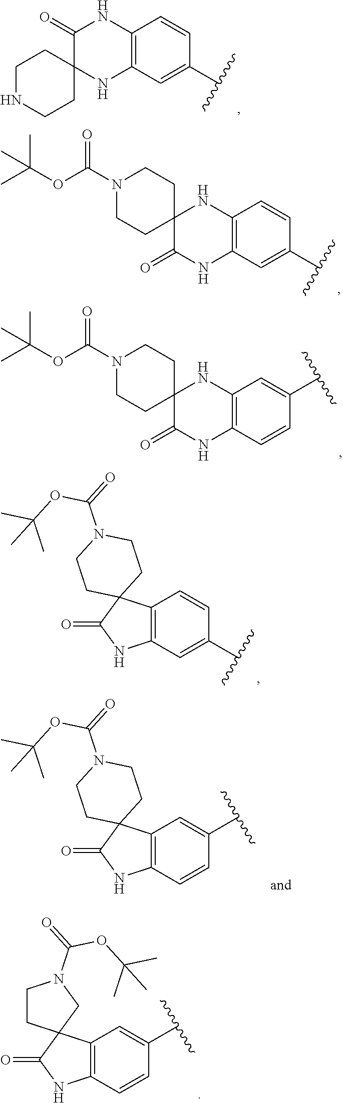
wherein R⁵ and R⁶ are independently selected from the group consisting of: —H, -D, —OH, —O—C₁-C₄ alkyl, C₁-C₄ alkyl, -halogen, CF₃, —NR′R″, —NHR⁷, —COOH, COR⁸, —NO₂, —CN, -Ph, SO₂NMe₂ and —CH₂NH₂, or are selected from the follow structures: 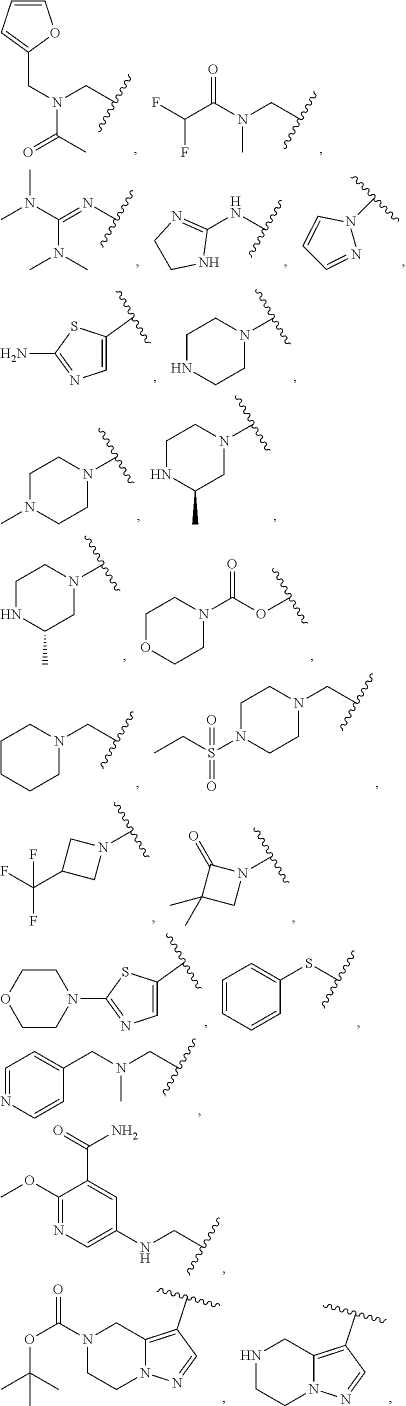 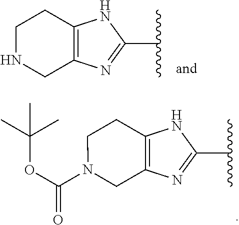
wherein R⁷ is —CH₂Ph, or is selected from the following substructures: 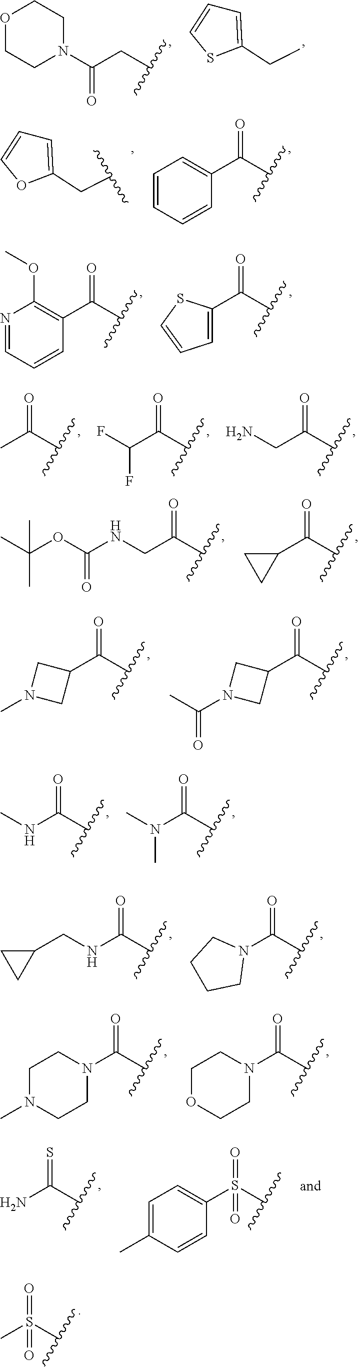
R⁸ is —NR′R″, C₁-C₄ alkyl or is selected from the following substructures: 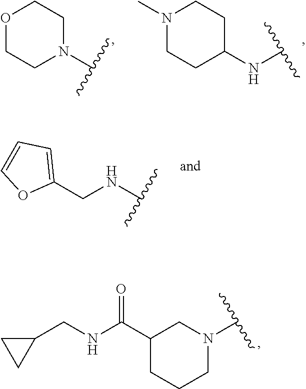
and R′ and R″ are independently —H or C₁-C₄ alkyl;
with the proviso that:
when A, D and E are N and B and M are C, then Y and Y′ are CH; X and X′ are independently CH or CF; Z is —S—; and R₁ is Me;
when A is C and B, D, E and M are N, then Y and Y′ are CH; X and X′ are independently CH or N; and R₁ is LR₂.

20. The compound according to claim 18, wherein A, D and E are N and B and Mare C or when A is C and B, D, E and M are N, and R² is a selected from the following substructures: 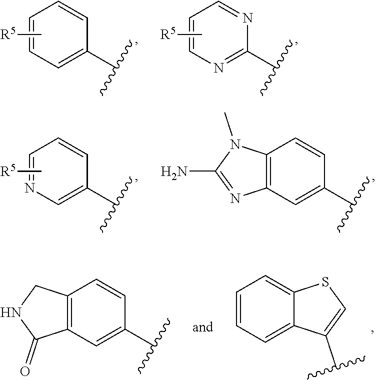
wherein:
R⁵ is —NH₂ or is selected from the following substructures:

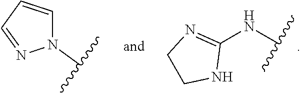 [SMILES: *n1cccn1 ; *NC1=NCCN1]

21. The compound according to claim 19, wherein the pentaheterocyclic core A-B-D-E-M is selected from the group consisting of 1,2,3-triazole, 2,5-disubstituted tetrazole 1,4-disubstituted pyrazole, Imidazole, 1,3,4-thiadiazole, 1,2,4-oxadiazole, 1,3,4-oxadiazole and isooxazole.

22. The compound according to claim 21, wherein the pentaheterocyclic core A-B-D-E-M is selected from the group consisting of 1,2,3-triazole, wherein B is C and M is N; 2,5-disubstituted tetrazole 1,4-disubstituted pyrazole, Imidazole, 1,3,4-thiadiazole; 1,2,4-oxadiazole, 1,3,4-oxadiazole and isooxazole.

23. The compound according to claim 22, wherein the pentaheterocyclic core A-B-D-E-M is selected from the group consisting of 1,2,3-triazole, wherein B is C and M is N; 1,3,4-thiadiazole; 1,2,4-oxadiazole, 1,3,4-oxadiazole and isooxazole.

24. The compound according to claim 19, wherein at least one of X, X′, Y and Y′ is CF or at least one of X and X′ is CCl.

25. The compound according to preceding claim 19, wherein Z is —CD₂-, —CF₂—, —CHR³—, —NH— or —S—;
wherein R³ is selected from the following substructures:

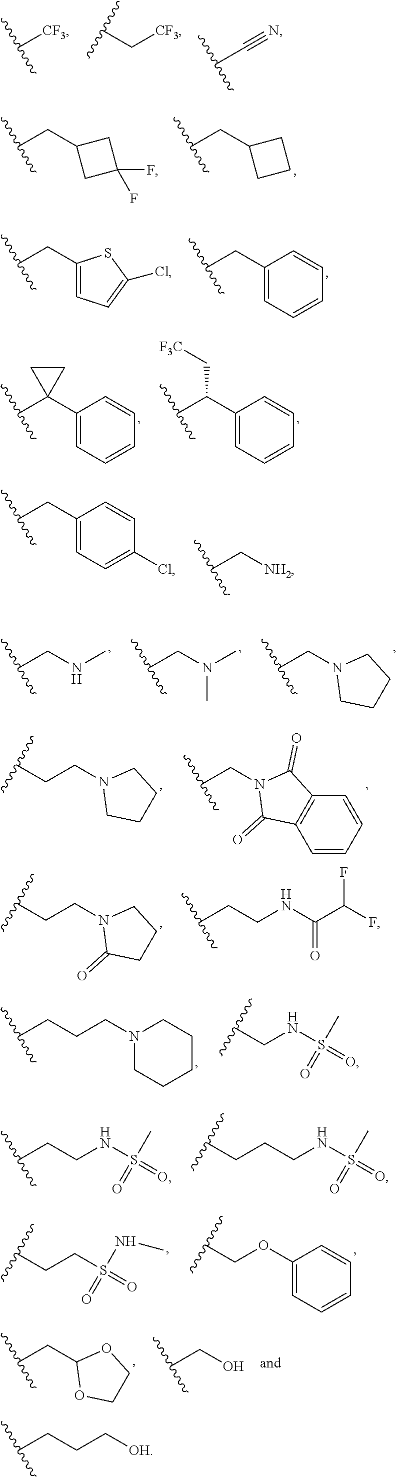

26. The compound according to any claim 19, wherein
R² is selected from group consisting of: 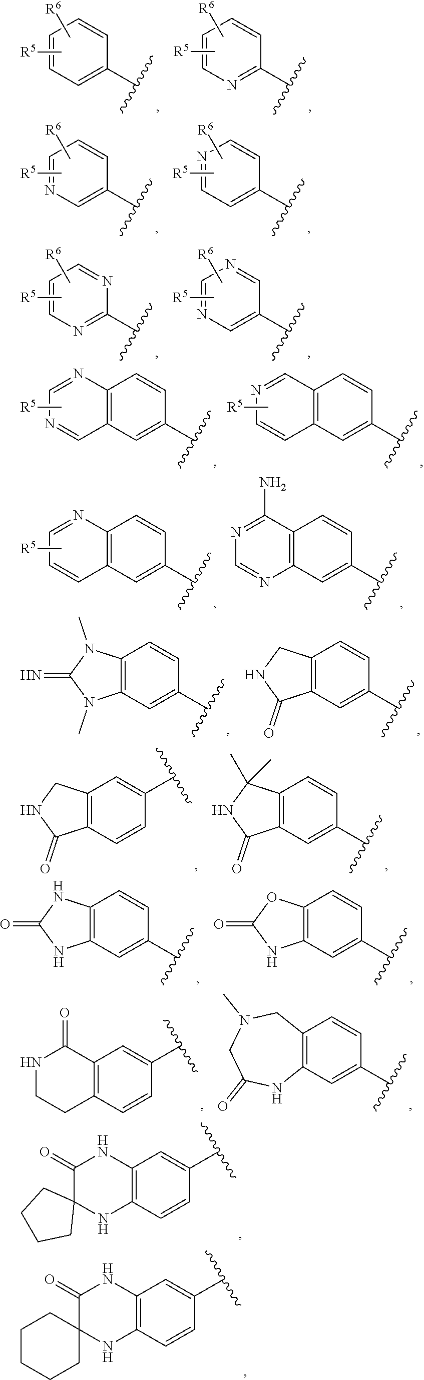 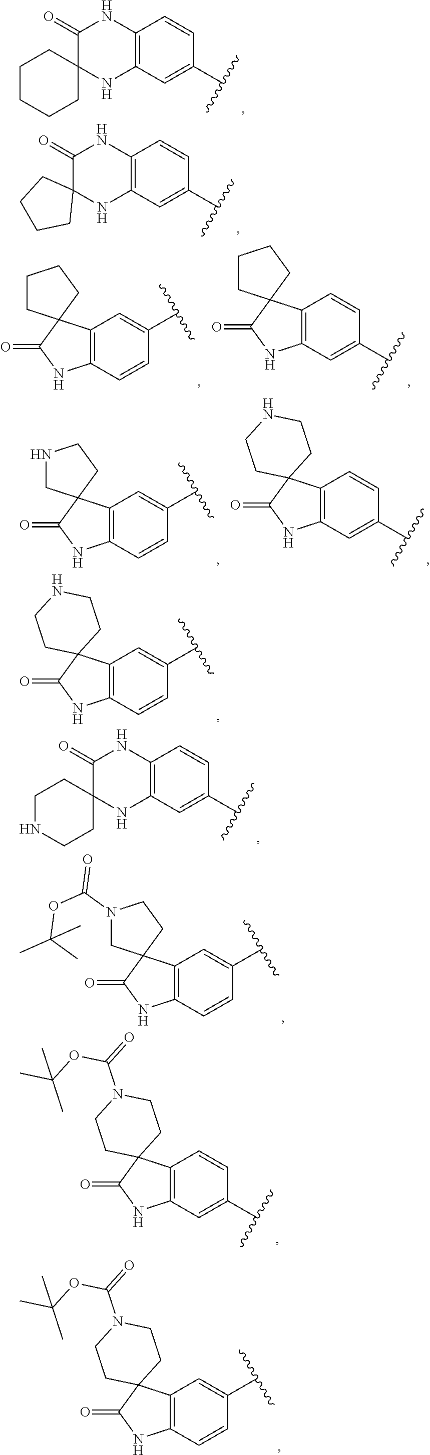 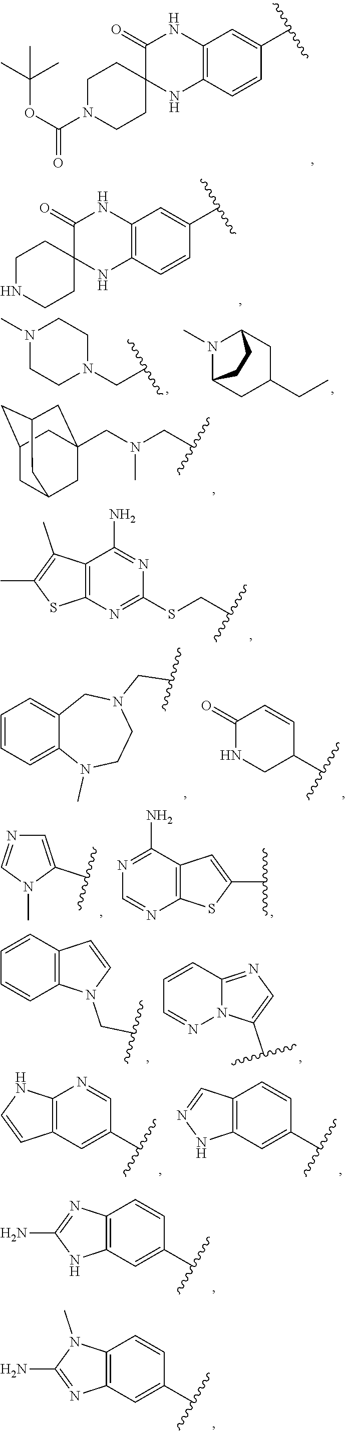 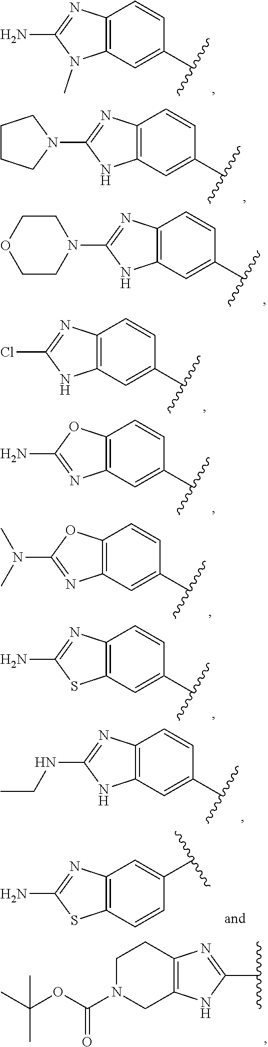
wherein at least one of R⁵ and R⁶ is selected from the group consisting of —OH, —NR′R″, —NHR⁷, —SO₂NMe₂, CH₂NH₂, —COR⁸ or is selected from the following substructures: 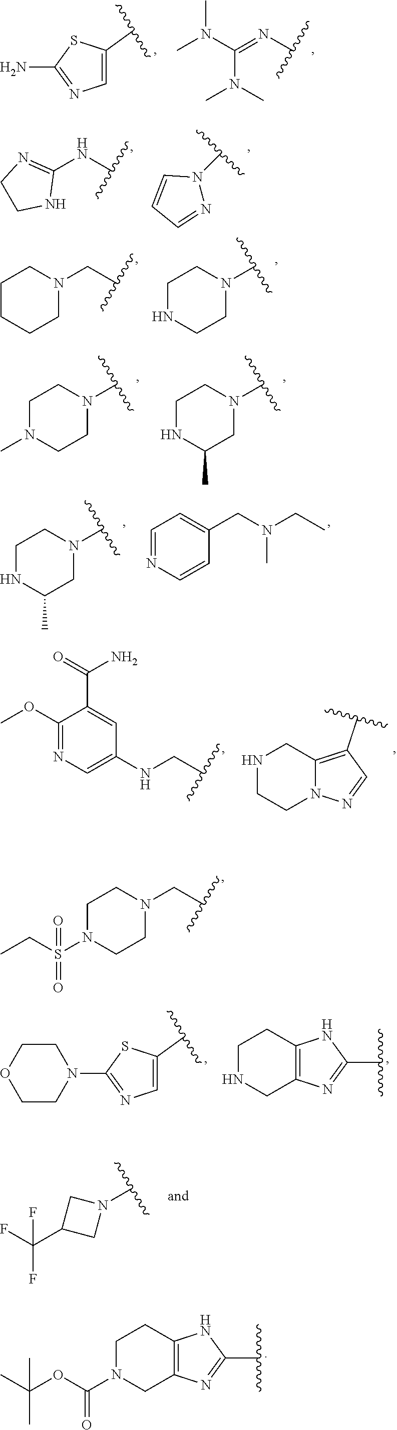
wherein R⁷ is selected from the following substructures: 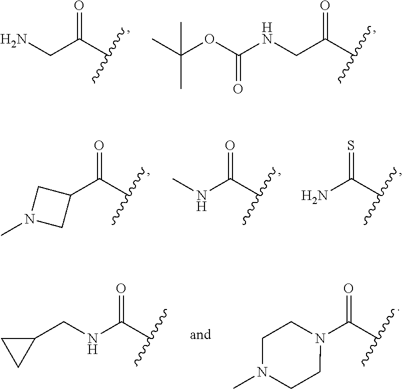
R⁸ is —NR′R″ or is selected from the follow substructures: 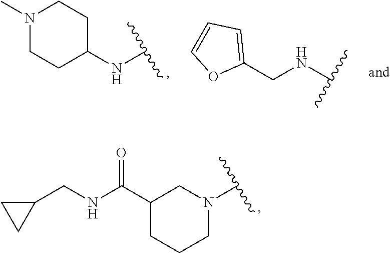
wherein R′ and R″ are gently —H or C₁-C₄ alkyl.

27. The compound according to claim 19, with the proviso that when B is N, Z is CHR³, wherein R³ is H or C₁-C₄ alkyl, L is absent and each of X, X′, Y, Y′ are CH or one or two of X, X′, Y, Y′ are N, then R² is not phenyl or pyridyl unsubstituted or substituted with one or more alkyl, alkoxy, thioalkoxy or halogenated derivatives thereof, or halogen, unsubstituted thiophenyl or furanyl.

28. The compound according to claim 19, with the proviso that the following compounds are excluded:
2-(difluoromethyl)-5-(4-((5-phenyl-1H-tetrazol-1-yl)methyl)phenyl)-1,3,4-oxadiazole;
2-(difluoromethyl)-5-(6-((4-phenyl-1H-imidazol-1-yl)methyl)pyridin-3-yl)-1,3,4-oxadiazole;
2-(difluoromethyl)-5-(4-((4-phenyl-1,2,3-triazol-1-yl)methyl)phenyl)-1,3,4-oxadiazole;
2-(4-((4-(4-chlorophenyl)-1H-1,2,3-triazol-1-yl)methyl)phenyl)-5-(difluoromethyl)-1,3,4-oxadiazole;
2-(difluoromethyl)-5-(4-((4-(4-(trifluoromethyl)phenyl)-1H-1,2,3-triazol-1-yl)methyl)phenyl)-1,3,4-oxadiazole;
2-(difluoromethyl)-5-(4-((4-(pyridin-4-yl)phenyl-1H-1,2,3-triazol-1-yl)methyl)phenyl)-1,3,4-oxadiazole;
2-(difluoromethyl)-5-(4-((4-(pyridin-3-yl)phenyl-1H-1,2,3-triazol-1-yl)methyl)phenyl)-1,3,4-oxadiazole;
2-(difluoromethyl)-5-(4-((4-(thiophen-3-yl)phenyl-1H-1,2,3-triazol-1-yl)methyl)phenyl)-1,3,4-oxadiazole;
2-(difluoromethyl)-5-(4-(1-(4-phenyl-1H-triazol-1-yl)ethyl)phenyl)-1,3,4-oxadiazole;
2-(difluoromethyl)-5-(4-((5-methyl-4-phenyl-1H-1,2,3-triazol-1-yl)methyl)phenyl)-1,3,4-oxadiazole;
2-(difluoromethyl)-5-(6-((4-phenyl-1H-1,2,3-triazol-1-yl)methyl)pyridin-3-yl)-1,3,4-oxadiazole;
2-(difluoromethyl)-5-(5-((4-phenyl-1H-1,2,3-triazol-1-yl)methyl)pyridin-2-yl)-1,3,4-oxadiazole;
2-(6-((4-(4-chlorophenyl)-1H-1,2,3-triazol-1-yl)methyl)pyridin-3-yl)-5-(difluoromethyl)-1,3,4-oxadiazol e;
2 (6-((4-(2-chlorophenyl)-1H-1,2,3-triazol-1-yl)methyl)pyridin-3-yl)-5-(difluoromethyl)-1,3,4 oxadiazole;
2-(6-((4-(3-chlorophenyl)-1H-1,2,3-triazol-1-yl)methyl)pyridin-3-yl)-5-(difluoromethyl)-1,3,4-oxadiazole;
2-(6-((4-(3,4-dichlorophenyl)-1H-1,2,3-triazol-1-yl)methyl)pyridin-3-yl)-5-(difluoromethyl)-1,3,4-oxadiazole;
2-(6-((4-(3,5-dichlorophenyl)-1H-1,2,3-triazol-1-yl)methyl)pyridin-3-yl)-5-(difluoromethyl)-1,3,4-oxadiazole;
2-(difluoromethyl 5-(6-((4-(2-fluorophenyl)-1H-1, 2, 3-triazol-1-yl)methyl)pyridin-3-yl)-1,3,4-oxadiazole;
2-(difluoromethyl)-5-(6-((4-(2,6-difluorophenyl)-1H-1,2,3-triazol-1-yl)methyl)pyridin-3-yl)-1,3,4-oxadiazole;
2-(6-((4-(3-chlorophenyl)-1H-1,2,3-triazol-1-yl)methyl)pyridin-3-yl)-5-(difluoromethyl)-1,3,4-oxadiazole; and
2-(difluoromethyl)-5-(6-((4-(3,5-difluorophenyl)-1H-1,2,3-triazol-1-yl)methyl)pyridin-3-yl)-1,3,4-oxadiazole.

29. The compound according to claim 19, wherein
X and X′ are independently CH, N or CF;
Y and Y′ are independently CH, N or CF;
A is C, N or S;
B is C or N,
D is C or N;
E is C, N or O;
M is C;
Z is CH₂ or CHR³, wherein
R³ is Me, or is selected from the following substructures: 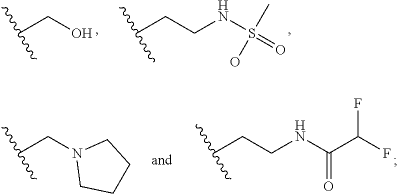
L is absent;
R² is selected from the group consisting of: 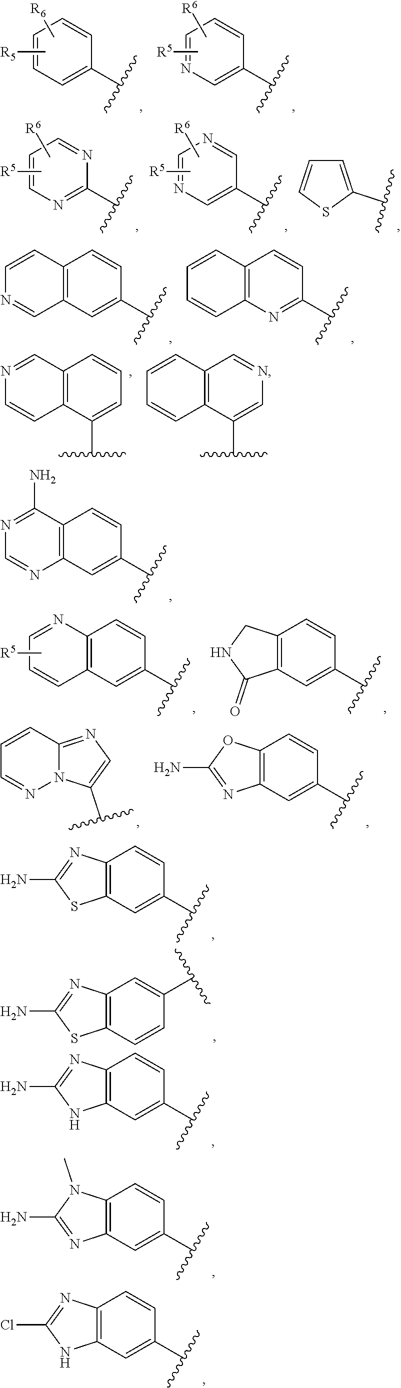 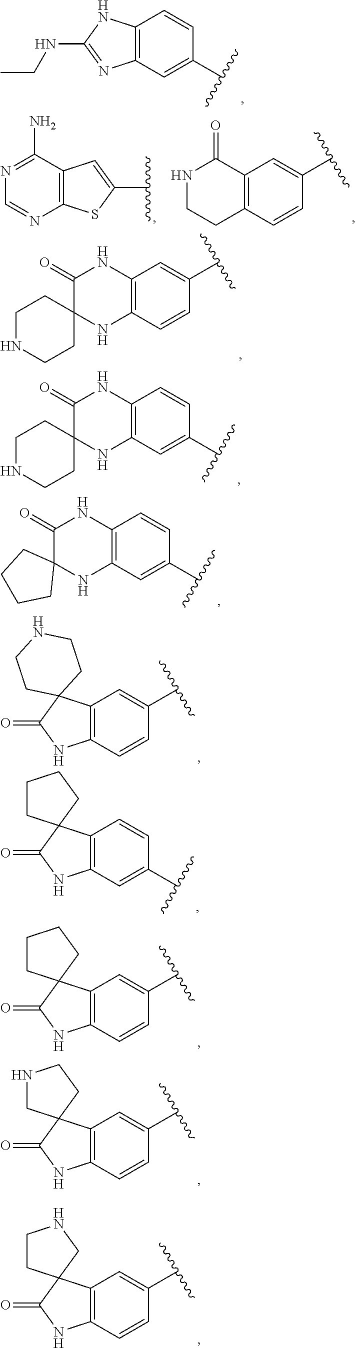 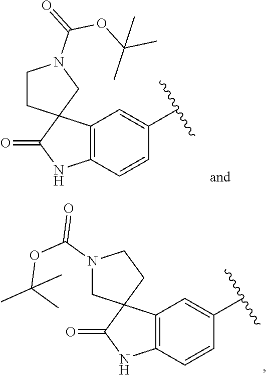
wherein R⁵ and R⁶ are independently selected from the group consisting of: —OH, —OMe, —Br, NH₂, —NHR⁷, —COR⁸, COCH₃, —CH₃, CH₂NH₂, or are selected from the following substructures: 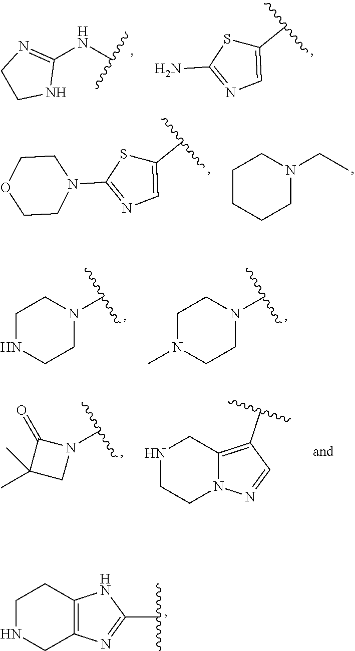
wherein R⁷ is Me, Et, or is selected from the following substructures: 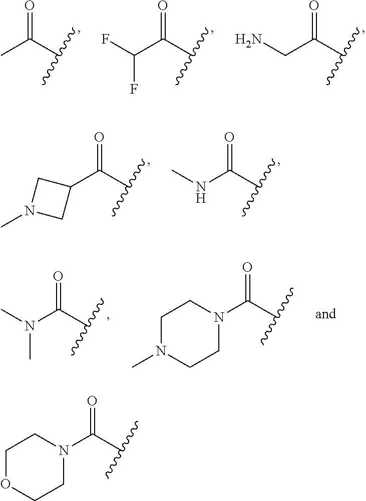
and R⁸ is —NH₂, NHEt, —NMe₂, or is selected from the following substructures:

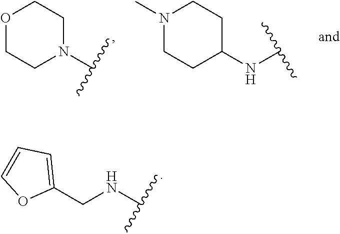

30. The compound according to claim 19, which is selected from:
6-(1-((5-(5-(difluoromethyl)-1,3,4-oxadiazol-2-yl)pyridin-2-yl)methyl)-1H-1,2,3-triazol-4-yl)benzo[d]thiazol-2-amine;
N-(5-(2-((5-(5-(difluoromethyl)-1,3,4-oxadiazol-2-yl)pyridin-2-yl)methyl)-2H-tetrazol-5-yl)-2-hydroxyphenyl)morpholine-4-carboxamide;
5-(1-((5-(5-(difluoromethyl)-1,3,4-oxadiazol-2-yl)pyridin-2-yl)methyl)-1H-1,2,3-triazol-4-yl benzo[d]thiazol-2-amine;
6-(2-((5-(5-(difluoromethyl)-1,3,4-oxadiazol-2-yl)pyridin-2-yl)methyl)-2H-tetrazol-5-yl)isoindolin-1-one;
5-(1-((5-(5-(difluoromethyl)-1,3,4-oxadiazol-2-yl)pyridin-2-yl)methyl)-1H-1,2,3-triazol-4-yl)pyridin-2-amine;
N-(3-(1-((5-(5-(difluoromethyl)-1,3,4-oxadiazol-2-yl)pyridin-2-yl)methyl)-1H-1,2,3-triazol-4-yl)phenyl)morpholine-4-carboxamide;
5-(2-((5-(5-(difluoromethyl)-1,3,4-oxadiazol-2-yl)pyridin-2-yl)methyl)-2H-tetrazol-5-yl)benzo[d]oxazol-2-amine;
5-(1-(4-(5-(difluoromethyl)-1,3,4-oxadiazol-2-yl)benzyl)-1H-1,2,3-triazol-4-yl)-1H-benzo[d]imidazol-2-amine;
2-(6-((4-(2-chloro-1H-benzo[d]imidazol-6-yl)-1H-1,2,3-triazol-1-ylmethyl)pyridin-3-yl)-5-(difluoromethyl)-1,3,4-oxadiazole;
N-(4-(1-(4-(5 difluoromethyl-1,3,4-(oxadiazol-2-yl)benzyl)-1H-1,2,3-triazol-4-yl)phenyl)-4,5-dihydro-1H-imidazol-2-amine;
5-(2-(4-(5-(difluoromethyl)-1,3,4-oxadiazol-2-yl)benzyl)-2H-tetrazol-5-yl)-1-methyl-1H-benzo[d]imidazol-2-amine;
5-(1-((5-(5-fluoromethyl)-1,3,4-oxadiazol-2-yl)pyridin-2-yl)methyl)-1H-imidazol-4-yl)pyridin-2-amine;
5-(1-((5-(5-(difluoromethyl)-1,3,4-oxadiazol-2-yl)pyridin-2-yl)methyl)-1H-pyrazol-4-yl)pyridin-2-amine;
6-(1-(4-(5-(difluoromethyl)-1,3,4-oxadiazol-2-yl)benzyl)-1H-1,2,3-triazol-4-yl)benzo[d]thiazol-2-amine;
5-(1-(4-(5-(difluoromethyl)-1,3,4-oxadiazol-2-yl)-3-fluorobenzyl)-1H-1,2,3-triazol-4-yl)pyridin-2-amine;
N-(3-(1-(4-(5-(difluoromethyl)-1,3,4-oxadiazol-2-yl)-3-fluorobenzyl)-1H-1,2,3-triazol-4-yl)phenyl)morpholine-4-carboxamide;
5-(1-((5-(5-(difluoromethyl)-1,3,4-oxadiazol-2-yl)pyridin-2-yl)methyl)-1H-1,2,3-triazol-4-yl)-1-methyl-1H-benzo[d]imidazol-2-amine;
6-(1-((5-(5-(difluoromethyl)-1,3,4-oxadiazol-2-yl)pyridin-2-yl)methyl)-1H-1,2,3-triazol-4-yl)-N-ethyl-1H-benzo[d]imidazol-2-amine;
5-(1-((5-(5-(difluoromethyl)-1,3,4-oxadiazol-2-yl)pyridin-2-yl)methyl)-1H-1,2,3-triazol-4-yl)spiro[indoline-3,4′-piperidin]-2-one;
N-(4-(1-((5-(5-(difluoromethyl)-1,3,4-oxadiazol-2-yl)pyridin-2-yl)methyl)-1H-imidazol-4-yl)phenyl)-4,5-dihydro-1H-imidazol-2-amine;
5-(1-(4-(5-(difluoromethyl)-1,3,4-oxadiazol-2-yl)-2,3-difluorobenzyl)-1H-1,2,3-triazol-4-yl)pyridin-2-amine;
N-(4-(1-(4-(5-(difluoromethyl)-1,3,4-oxadiazol-2-yl)benzyl)-1H-imidazol-4-yl)phenyl)-4,5-dihydro-1H-imidazol-2-amine;
5-(1-(4-(5-(difluoromethyl)-1,3,4-oxadiazol-2-yl)benzyl)-1H-1,2,3-triazol-4-yl)-1-methyl-1H-benzo[d]imidazol-2-amine;
N-(4-(1-((5-(5-(difluoromethyl)-1,3,4-oxadiazol-2-yl)pyridin-2-yl)methyl)-1H-1,2,3-triazol-4-yl)phenyl)-4,5-dihydro-1H-imidazol-2-amine;
N-(5-(2-(4-(5-(difluoromethyl-1,3,4-oxadiazol-2-yl)benzyl)-2H-tetrazol-5-yl)-2-hydroxyphenyl)morpholine-4-carboxamide;
5′-(1-((5-(5-(difluoromethyl)-1,3,4-oxadiazol-2-yl)pyridin-2-yl)methyl)-1H-1,2,3-triazol-4-yl)spiro[cyclopentane-1,3′-indolin]-2′-one;
7′-(2-((5-(5-(difluoromethyl)-1,3,4-oxadiazol-2-yl)pyridin-2-yl)methyl)-2H-tetrazol-5-yl)-1′,4′-dihydro-3′H-spiro[cyclopentane-1,2′-quinoxalin]-3′-one;
5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one;
3-(2-((5-(5-(difluoromethyl)-1,3,4-oxadiazol-2-yl)pyridin-2-yl)methyl)-2H-tetrazol-5-yl)benzamide;
6-(1-((5-(5-(difluoromethyl)-1,3,4-oxadiazol-2-yl)pyridin-2-yl)methyl)-1H-1,2,3-triazol-4-yl)-1H-benzo[d]imidazol-2-amine;
3-(5-(2-(4-(5-(difluoromethyl)-1,3,4-oxadiazol-2-yl)benzyl)-2H-tetrazol-5-yl)-2-hydroxyphenyl)-1,1-dimethylurea;
(R)-5-(1-(1-(5-(5-(difluoromethyl)-1,3,4-oxadiazol-2-yl)pyridin-2-yl)ethyl)-1H-1,2,3-triazol-4-yl)pyridin-2-amine;
(4-(2-(4-(5-(difluoromethyl)-1,3,4-oxadiazol-2-yl)-2,6-difluorobenzyl)-2H-tetrazol-5-yl)phenyl)methanamine;
6-(1-((5-(5-(difluoromethyl)-1,3,4-oxadiazol-2-yl)pyridin-2-yl)methyl)-1H-1,2,3-triazol-4-yl)-N-methylquinolin-2-amine;
2-amino-4-(2-(4-(5-(difluoromethyl)-1,3,4-oxadiazol-2-yl)benzyl)-2H-tetrazol-5-yl)phenol;
7′-(1-((5-(5-(difluoromethyl)-1,3,4-oxadiazol-2-yl)pyridin-2-yl)methyl)-1H-1,2,3-triazol-4-yl)-1′,4′-dihydro-3′H-spiro[piperidine-4,2′-quinoxalin]-3′-one;
N-(3-(1-((5-(5-(difluoromethyl)-1,3,4-oxadiazol-2-yl)pyridin-2-yl)methyl)-1H-1,2,3-triazol-4-yl)phenyl)acetamide;
5-(3-(2-((5-(5-(difluoromethyl)-1,3,4-oxadiazol-2-yl)pyridin-2-yl)methyl)-2H-tetrazol-5-yl)phenyl)thiazol-2-amine;
5-(1-(4-(5-(difluoromethyl)-1,3,4-oxadiazol-2-yl)-2,5-difluorobenzyl)-1H-1,2,3-triazol-4-yl)pyridin-2-amine;
6-(2-(4-(5-(difluoromethyl)-1,3,4-oxadiazol-2-yl)-2-fluorobenzyl)-2H-tetrazol-5-yl)isoindolin-1-one;
6′-(1-((5-(5-(difluoromethyl)-1,3,4-oxadiazol-2-yl)pyridin-2-yl)methyl)-1H-1,2,3-triazol-4-yl)-1′,4′-dihydro-3′H-spiro[piperidine-4,2′-quinoxalin]-3′-one;
5-(1-(4-(5-(difluoromethyl)-1,3,4-oxadiazol-2-yl)-2,6-difluorobenzyl)-1H-1,2,3-triazol-4-yl)pyridin-2-amine;
(4-(2-(4-(5-(difluoromethyl)-1,3,4-oxadiazol-2-yl)benzyl)-2H-tetrazol-5-yl)phenyl)methanamine;
(4-(1-(4-(5-(difluoromethyl)-1,3,4-oxadiazol-2-yl)-2,8-difluorobenzyl)-1H-1,2,3-triazol-4-yl)phenyl)methanamine;
5-(2-(4-(5-(difluoromethyl)-1,3,4-oxadiazol-2-yl)-2,6-difluorobenzyl)-2H-tetrazol-5-yl)pyridin-2-amine;
5-(1-(4-(5-(difluoromethyl)-1,3,4-oxadiazol-2-yl)benzyl)-1H-1,2,3-triazol-4-yl)spiro[indoline-3,4′-piperidin]-2-one;
N-(3-(1-(4-(5-(difluoromethyl)-1,3,4-oxadiazol-2-yl)-2-fluorobenzyl)-1H-1,2,3-triazol-4-yl)phenyl)morpholine-4-carboxamide;
5-(1-(4-(5-(difluoromethyl)-1,3,4-oxadiazol-2-yl)-2-fluorobenzyl)-1H-1,2,3-triazol-4-yl)pyridin-2-amine;
5-(1-(4-(5-(difluoromethyl)-1,3,4-oxadiazol-2-yl)benzyl)-1H-1,2,3-triazol-4-yl)spiro[indoline-3,3′-pyrrolidin]-2-one;
3-(2-(4-(5-(difluoromethyl)-1,3,4-oxadiazol-2-yl)-2-fluorobenzyl)-2H-tetrazol-5-yl)benzamide;
N-(3-(1-((5-(5-(difluoromethyl)-1,3,4-oxadiazol-2-yl)pyridin-2-yl)methyl)-1H-1,2,3-triazol-4-yl)phenyl)-4-methylpiperazine-1-carboxamide;
5-(1-(4-(5-(difluoromethyl)-1,3,4-oxadiazol-2-yl)benzyl)-1H-1,2,3-triazol-4-yl)pyridin-2-amine;
2-(difluoromethyl)-5-(6-((4-(2-methoxypyridin-3-yl)-1H-1,2,3-triazol-1-yl)methyl)pyridin-3-yl)-1,3,4-oxadiazole;
3-(2-(4-(5-(difluoromethyl)-1,3,4-oxadiazol-2-yl)-2,6-difluorobenzyl)-2H-tetrazol-5-yl)benzamide;
6-(2-(4-(5-(difluoromethyl)-1,3,4-oxadiazol-2-yl)benzyl)-2H-tetrazol-5-yl)isoindolin-1-one;
4-(2-(4-(5-(difluoromethyl)-1,3,4-oxadiazol-2-yl)benzyl)-2H-tetrazol-5-yl)phenol;
6-(2-(4-(5-(difluoromethyl)-1,3,4-oxadiazol-2-yl)-2,6-difluorobenzyl)-2H-tetrazol-5-yl)isoindolin-1-one;
2-(difluoromethyl)-5-(4-(3-(4-methylpiperazin-1-yl)phenyl)-2H-tetrazol-2-yl)methyl)phenyl)-1,3,4-oxadiazole;
5-(1-(1-(5-(5-(difluoromethyl)-1,3,4-oxadiazol-2-yl)pyridin-2-yl)ethyl)-1H-1,2,3-triazol-4-yl)pyridin-2-amine;
6-(1-(4-(5-(difluoromethyl)-1,3,4-oxadiazol-2-yl)benzyl)-1H-1,2,3-triazol-4-yl)-N-ethyl-1H-benzo[d]imidazol-2-amine;
5′-(1-(4-(5-(difluoromethyl)-1,3,4-oxadiazol-2-yl)benzyl)-1H-1,2,3-triazol-4-yl)spiro[cyclopentane-1,3-indolin]-2′-one;
N-(3-(4-(6-aminopyridin-3-yl)-1H-1,23-triazol-1-yl)-3-(4-(5-(difluoromethyl)-1,3,4-oxadiazol-2-yl)phenyl)propyl)methanesulfonamide;
N-(3-(1-(4-(5-(difluoromethyl)-1,3,4-oxadiazol-2-yl)-2,6-difluorobenzyl)-1H-1,2,3-triazol-4-yl)phenyl)-4-methylpiperazine-1-carboxamide;
5-(2-(4-(5-(difluoromethyl)-1,3,4-oxadiazol-2-yl)benzyl)-2H-tetrazol-5-yl)pyridin-2-amine;
5-(2-(4-(5-(difluoromethyl)-1,3,4-oxadiazol-2-yl)benzyl)-2H-tetrazol-5-yl)-2-methylpyridin-3-amine;
N-(3-(1-(1-(5-(5-(difluoromethyl)-1,3,4-oxadiazol-2-yl)pyridin-2-yl)ethyl)-1H-1,2,3-triazol-4-yl)phenyl)morpholine-4-carboxamide;
2-(3,5-difluoro-4-((4-(imidazo[1,2-b]pyridazin-3-yl)-1H-1,2,3-triazol-1-yl)methyl)phenyl)-5-(difluoromethyl)-1,3,4-oxadiazole;
N-(5-(2-((5-(5-(difluoromethyl)-1,3,4-oxadiazol-2-yl)pyrimidin-2-yl)methyl)-2H-tetrazol-5-yl)pyridin-2-yl)-2,2-difluoroacetamide;
(3-(2-((5-(5-(difluoromethyl)-1,3,4-oxadiazol-2-yl)pyridin-2-yl)methyl)-2H-tetrazol-5-yl)phenyl)(morpholino)methanone;
N-(3-(1-(4-(5-(difluoromethyl)-1,3,4-oxadiazol-2-yl)-2,6-difluorobenzyl)-1H-1,2,3-triazol-4-yl)phenyl)acetamide;
N-(3-(2-(4-(5-(difluoromethyl)-1,3,4-oxadiazol-2-yl)benzyl)-2H-tetrazol-5-yl)phenyl)morpholine-4-carboxamide;
2-amino-5-(2-(4-(5-(difluoromethyl)-1,3,4-oxadiazol-2-yl)benzyl)-2H-tetrazol-5-yl)benzamide;
5-(1-(4-(5-(difluoromethyl)-1,3,4-oxadiazol-2-yl)-2,6-difluorobenzyl)-1H-1,2,3-triazol-4-yl)pyridin-3-amine;
2-(difluoromethyl)-5-(6-((4-(imidazo[1,2-b]pyridazin-3-yl)-1H-1,2,3-triazol-1-yl)methyl)pyridin-3-yl)-1,3,4-oxadiazole;
3-(2-(4-(5-(difluoromethyl)-1,3,4-oxadiazol-2-yl)benzyl)-2H-tetrazol-5-yl)benzamide;
2-amino-5-(2-(4-(5-(difluoromethyl)-1,3,4-oxadiazol-2-yl)benzyl)-2H-tetrazol-5-yl)nicotinamide;
5-(2-((5-(5-(difluoromethyl)-1,3,4-oxadiazol-2-yl)pyrimidin-2-yl)methyl)-2H-tetrazol-5-yl)pyridin-2-amine;
N-(3-(1-(4-(5-(difluoromethyl)-1,3,4-oxadiazol-2-yl)-2,6-difluorobenzyl)-1H-1,2,3-triazol-4-yl)phenyl)morpholine-4-carboxamide;
5-(2-(4-(5-(difluoromethyl)-1,3,4-oxadiazol-2-yl)benzyl)-2H-tetrazol-5-yl)pyrimidin-2-amine;
3-(2-(4-(5-(difluoromethyl)-1,3,4-oxadiazol-2-yl)benzyl)-2H-tetrazol-5-yl)-N-(1-methylpiperidin-4-yl)benzamide;
3-(2-(4-(5-(difluoromethyl)-1,3,4-oxadiazol-2-yl)benzyl)-2H-tetrazol-5-yl)-N,N-dimethylbenzamide;
2-(4-((5-(5-bromopyridin-3-yl)-2H-tetrazol-2-yl)methyl)phenyl)-5-(difluoromethyl)-1,3,4-oxadiazole;
7-(2-(4-(5-(difluoromethyl)-1,3,4-oxadiazol-2-yl)benzyl)-2H-tetrazol-5-yl)-3,4-dihydroisoquinolin-1 (2H)-one;
7-(1-(4-(5-(difluoromethyl)-1,3,4-oxadiazol-2-yl)benzyl)-1H-1,2,3-triazol-4-yl)quinazolin-4-amine;
2-(difluoromethyl)-5-(6-((4-(thiophen-2-yl)-1H-1,2,3-triazol-1-yl)methyl)pyridin-3-yl)-1,3,4-oxadiazole;
N-(3-(1-(4-(5-(difluoromethyl)-1,3,4-oxadiazol-2-yl)-2,6-difluorobenzyl)-1H-1,2,3-triazol-4-yl)phenyl)-1-methylazetidine-3-carboxamide;
2-(difluoromethyl)-5-(4-((5-(4-(piperidin-1-ylmethyl)phenyl)-2H-tetrazol-2-yl)methyl)phenyl)-1,3,4-oxadiazole;
N-(5-(1-((5-(5-(difluoromethyl)-1,3,4-oxadiazol-2-yl)pyrimidin-2-yl)methyl)-1H-1,2,3-triazol-4-yl)pyridin-2-yl)-2,2-difluoroacetamide;
3-(2-(4-(5-(difluoromethyl)-1,3,4-oxadiazol-2-yl)-3-fluorobenzyl)-2H-tetrazol-5-yl)benzamide;
5-(2-(4-(5-(difluoromethyl)-1,3,4-oxadiazol-2-yl)benzyl)-2H-tetrazol-5-yl)pyridin-3-amine;
3-(2-(4-(5-(difluoromethyl)-1,3,4-oxadiazol-2-yl)benzyl)-2H-tetrazol-5-yl)-N-ethylbenzamide;
1-(3-(1-(4-(5-(difluoromethyl)-1,3,4-oxadiazol-2-yl)-2,6-difluorobenzyl)-1H-1,2,3-triazol-4-yl)phenyl)-3,3-dimethylazetidin-2-one;
(3-(2-(4-(5-(difluoromethyl)-1,3,4-oxadiazol-2-yl)-2-fluorobenzyl)-2H-tetrazol-5-yl)phenyl)(morpholino)methanone;
2-(4-(6-aminopyridin-3-yl)-1H-1,2,3-triazol-1-yl)-2-(4-(5-(difluoromethyl)-1,3,4-oxadiazol-2-yl)phenyl)ethan-1-ol;
N-(5-(2-(4-(5-(difluoromethyl)-1,3,4-oxadiazol-2-yl)-2,6-difluorobenzyl)-2H-tetrazol-5-yl-2-hydroxyphenyl)morpholine-4-carboxamide;
3-(2-(4-(5-(difluoromethyl)-1,3,4-oxadiazol-2-yl)benzyl)-2H-tetrazol-5-yl)-N-(furan-2-ylmethyl)benzamide;
6-(2-(4-(5-(difluoromethyl)-1,3,4-oxadiazol-2-yl)-3-fluorobenzyl)-2H-tetrazol-5-yl)isoindolin-1-one;
N-(3-(2-(4-(5-(difluoromethyl)-1,3,4-oxadiazol-2-yl)-2,6-difluorobenzyl)-2H-tetrazol-5-yl)phenyl)morpholine-4-carboxamide;
5-(2-(4-(5-(difluoromethyl)-1,3,4-oxadiazol-2-yl)benzyl)-2H-tetrazol-5-yl)-N-ethylpyridin-2-amine;
(4-(3-(4-(5-(difluoromethyl)-1,3,4-oxadiazol-2-yl)-2,6-difluorobenzyl)-1,2,4-oxadiazol-5-yl)phenyl)methanamine;
(5-(1-(4-(5-(difluoromethyl)-1,3,4-oxadiazol-2-yl)-2,6-difluorobenzyl)-1H-1,2,3-triazol-4-yl)pyridin-2-yl)methanamine;
N-(5-(5-((5-(5-(difluoromethyl)-1,3,4-oxadiazol-2-yl)pyridin-2-yl)methyl)-1,3,4-thiadiazol-2-yl)pyridin-2-yl)-2,2-difluoroacetamide;
2-(difluoromethyl)-5-(4-((5-(4-(piperazin-1-yl)phenyl)-2H-tetrazol-2-yl)methyl)phenyl)-1,3,4-oxadiazole;
N-(3-(1-(1-(4-(5-(difluoromethyl)-1,3,4-oxadiazol-2-yl)phenyl)ethyl)-1H-1,2,3-triazol-4-yl)phenyl)morpholine-4-carboxamide;
2-(3,5-difluoro-4-((4-(2-methylpyridin-3-yl)-1H-1,2,3-triazol-1-yl)methyl)phenyl)-5-(difluoromethyl)-1,3,4-oxadiazole;
(R)-5-(1-(1-(4-(5-(difluoromethyl)-1,3,4-oxadiazol-2-yl)phenyl)ethyl)-1H-1,2,3-triazol-4-yl)pyridin-2-amine;
6-(2-((5-(5-(difluoromethyl)-1,3,4-oxadiazol-2-yl)pyrimidin-2-yl)methyl)-2H-tetrazol-5-yl)isoindolin-1-one;
2-(difluoromethyl)-5-(4-((5-(3-(4,5,6,7-tetrahydro-1H-imidazo[4,5-c]pyridin-2-yl)phenyl)-2H-tetrazol-2-yl)methyl)phenyl)-1,3,4-oxadiazole;
6-(1-(4-(5-(difluoromethyl)-1,3,4-oxadiazol-2-yl)benzyl)-1H-1,2,3-triazol-4-yl)isoindolin-1-one;
7′-(2-(4-(5-(difluoromethyl)-1,3,4-oxadiazol-2-yl)benzyl)-2H-tetrazol-5-yl)-1′,4′-dihydro-3H-spiro[cyclopentane-1,2′-quinoxalin]-3′-one;
2-(difluoromethyl)-5-(4-((5-(4-(4,5,6,7-tetrahydropyrazolo[1,5-a]pyrazin-3-yl)phenyl)-2H-tetrazol-2-yl)methyl)phenyl)-1,3,4-oxadiazole;
(3-(2-(4-(5-(difluoromethyl)-1,3,4-oxadiazol-2-yl)-2,6-difluorobenzyl)-2H-tetrazol-5-yl)phenyl)(morpholino)methanone;
2-(difluoromethyl)-5-(4-((5-(quinolin-2-yl)-2H-tetrazol-2-yl)methyl)phenyl)-1,3,4-oxadiazole;
3-(1-(4-(5-(difluoromethyl)-1,3,4-oxadiazol-2-yl)-2,8-difluorobenzyl)-1H-1,2,3-triazol-4-yl)-N-ethylaniline;
2-(difluoromethyl)-5-(6-((4-(2-methylpyridin-3-yl)-1H-1,2,3-triazol-1-yl)methyl)pyridin-3-yl)-1,3,4-oxadiazole;
4-(2-(4-(5-(difluoromethyl)-1,3,4-oxadiazol-2-yl)benzyl)-2H-tetrazol-5-yl)benzamide;
5-(1-(1-(4-(5-(difluoromethyl)-1,3,4-oxadiazol-2-yl)phenyl)ethyl)-1H-1,2,3-triazol-4-yl)pyridin-2-amine;
2-(difluoromethyl)-5-(4-((5-(isoquinolin-4-yl-2H-tetrazol-2-yl)methyl)phenyl)-1,3,4-oxadiazole;
N-(3-(1-((5-(5-(difluoromethyl)-1,3,4-oxadiazol-2-yl)pyridin-2-yl)methyl)-1H-pyrazol-4-yl)phenyl)morpholine-4-carboxamide;
(3-(2-(4-(5-(difluoromethyl)-1,3,4-oxadiazol-2-yl)benzyl)-2H-tetrazol-5-yl)phenyl)morpholino)methanone;
4-(1-(4-(5-(difluoromethyl)-1,3,4-oxadiazol-2-yl)benzyl)-1H-1,2,3-triazol-4-yl)aniline;
2-(3,5-difluoro-4-((4-(thiophen-2-yl)-1H-1,2,3-triazol-1-yl)ethyl)phenyl)-5-(difluoromethyl)-1,3,4-oxadiazole;
6′-(1-((5-(5-(difluoromethyl)-1,3,4-oxadiazol-2-yl)pyridin-2-yl)methyl)-1H-1,2,3-triazol-4-yl)spiro[cyclopentane-1,3′-indolin]-2′-one;
5-(1-(1-(4-(5-(difluoromethyl)-1,3,4-oxadiazol-2-yl)phenyl)-2-(pyrrolidin-1-yl)ethyl)-1H-1,2,3-triazol-4-yl)pyridin-2-amine;
5-(1-((5-(5-(difluoromethyl)-1,3,4-oxadiazol-2-yl)pyrimidin-2-yl)methyl)-1H-1,2,3-triazol-4-yl)pyridin-2-amine;
N-(5-(1-((5-(5-(difluoromethyl)-1,3,4-oxadiazol-2-yl)pyridin-2-yl)methyl)-1H-imidazol-4-yl)pyridin-2-yl)-2,2-difluoroacetamide;
2-(difluoromethyl)-5-(4-((5-(isoquinolin-7-yl)-2H-tetrazol-2-yl)methyl)phenyl)-1,3,4-oxadiazole;
2-(difluoromethyl)-S-(4-((5-(3,4-dimethoxyphenyl)-2H-tetrazol-2-yl)methyl)phenyl)-1,3,4-oxadiazole;
3-(2-(4-(5-(difluoromethyl)-1,3,4-oxadiazol-2-yl)benzyl)-2H-tetrazol-5-yl)aniline;
4-(5-(3-(2-((5-(5-(difluoromethyl)-1,3,4-oxadiazol-2-yl)pyridin-2-yl)methyl)-2H-tetrazol-S-yl)phenyl)thiazol-2-yl)morpholine;
2-(difluoromethyl)-5-(4-((4-(2-methoxypyridin-3-yl)-1H-1,2,3-triazol-1-yl)methyl)phenyl)-1,3,4-oxadiazole;
5-(1-(4-(5-(difluoromethyl)-1,3,4-oxadiazol-2-yl)benzyl)-1H-1,2,3-triazol-4-yl)benzo[d]thiazol-2-amine;
N-(5-(2-(4-(5-(difluoromethyl)-1,3,4-oxadiazol-2-yl)benzyl)-2H-tetrazol-5-yl)-2-methylpyridin-3-yl)acetamide;
5-(1-(2-chloro-4-(5-(difluoromethyl)-1,3,4-oxadiazol-2-yl)benzyl)-1H-1,2,3-triazol-4-yl)pyridin-2-amine;
5-(5-(4-(5-(difluoromethyl)-1,3,4-oxadiazol-2-yl)benzyl)-1,2,4-oxadiazol-3-yl)pyridin-2-amine;
2-(4-((4-(2-chloro-1H-benzo[d]imidazol-6-yl)-1H-1,2,3-triazol-1-yl)methyl)phenyl)-5-(difluoromethyl)-1,3,4-oxadiazole;
(3-(2-(4-(5-(difluoromethyl)-1,3,4-oxadiazol-2-yl)-3-fluorobenzyl)-2H-tetrazol-5-yl)phenyl)morpholino)methanone;
5-((4-(1-(4-(5-(difluoromethyl)-1,3,4-oxadiazol-2-yl)benzyl)-1H-1,2,3-triazol-4-yl)benzyl)amino)-2-methoxynicotinamide;
N-(3-(1-(4-(5-(difluoromethyl)-1,3,4-oxadiazol-2-yl)benzyl)-1H-1,2,3-triazol-4-yl)phenyl)acetamide;
1-(3-(2-(4-(5-(difluoromethyl)-1,3,4-oxadiazol-2-yl)benzyl)-2H-tetrazol-5-yl)phenyl)ethan-1-one;
5-(3-(4-(5-(difluoromethyl)-1,3,4-oxadiazol-2-yl)-2,6-difluorobenzyl)-1,2,4-oxadiazol-5-yl)pyridin-2-amine;
6-(1-(4-(5-(difluoromethyl)-1,3,4-oxadiazol-2-yl)benzyl)-1H-1,2,3-triazol-4-yl)-N-methylquinolin-2-amine;
(R)-5-(1-(1-(4-(5-(difluoromethyl)-1,3,4-oxadiazol-2-yl)phenyl)butyl)-1H-1,2,3-triazol-4-yl)pyridin-2-amine;
2-amino-N-(3-(1-(4-(5-(difluoromethyl)-1,3,4-oxadiazol-2-yl)-2,6-difluorobenzyl)-1H-1,2,3-triazol-4-yl)phenyl)acetamide;
N-(3-(3-(4-(5-(difluoromethyl)-1,3,4-oxadiazol-2-yl)-2,6-difluorobenzyl)-1,2,4-oxadiazol-5-yl)phenyl)morpholine-4-carboxamide;
N-(3-(1-(4-(5-(difluoromethyl)-1,3,4-oxadiazol-2-yl)benzyl)-1H-1,2,3-triazol-4-yl)phenyl)-4-methylpiperazine-1-carboxamide;
2-(difluoromethyl)-5-(4-((5-(1-(pyridin-2-yl)cyclopropyl)-2H-tetrazol-2-yl)methyl)phenyl)-1,3,4-oxadiazole;
2-(difluoromethyl)-5-(4-((5-(6-(piperazin-1-yl)pyridin-3-yl)-2H-tetrazol-2-yl)methyl)phenyl)-1,3,4-oxadiazole;
N-(3-(1-(4-(5-(difluoromethyl)-1,3,4-oxadiazol-2-yl)benzyl)-1H-1,2,3-triazol-4-yl)phenyl)-1-methylazetidine-3-carboxamide;
2-(difluoromethyl)-5-(4-((5-(2-nitrophenyl)-2H-tetrazol-2-yl)methyl)phenyl)-1,3,4-oxadiazole;
5-(1-(4-(5-(difluoromethyl)-1,3,4-oxadiazol-2-yl)benzyl)-1H-imidazol-4-yl)pyridin-2-amine;
5-(2-(4-(5-(difluoromethyl)-1,3,4-oxadiazol-2-yl)benzyl)-2H-tetrazol-5-yl)benzo[d]oxazol-2-amine;
2-(difluoromethyl)-5-(4-((5-(isoquinolin-5-yl)-2H-tetrazol-2-yl)methyl)phenyl)-1,3,4-oxadiazole;
5-((4-(1-(4-(5-(difluoromethyl)-1,3,4-oxadiazol-2-yl)-2,6-difluorobenzyl)-1H-1,2,3-triazol-4-yl)benzyl)amino)-2-methoxynicotinamide;
(5-(1-(4-(5-(difluoromethyl)-1,3,4-oxadiazol-2-yl)benzyl)-1H-1,2,3-triazol-4-yl)pyridin-2-yl)methanamine;
N-(3-(2-(4-(5-(difluoromethyl)-1,3,4-oxadiazol-2-yl)benzyl)-2H-tetrazol-5-yl)phenyl)benzamide;
7′-(2-(4-(5-(difluoromethyl)-1,3,4-oxadiazol-2-yl)benzyl)-2H-tetrazol-5-yl)-1′,4′-dihydro-3′H-spiro[cyclohexane-1,2′-quinoxalin]-3′-one;
5-(1-(1-(4-(5-(difluoromethyl)-1,3,4-oxadiazol-2-yl)phenyl)-3,3,3-trifluoropropyl)-1H-1,2,3-triazol-4-yl)pyridin-2-amine;
(R)-2-(difluoromethyl)-5-(4-((5-(6-(3-methylpiperazin-1-yl)pyridin-3-yl)-2H-tetrazol-2-yl)methyl)phenyl 1,3,4-oxadiazole;
2-amino-4-(2-(4-(5-(difluoromethyl)-1,3,4-oxadiazol-2-yl)-2,6-difluorobenzyl)-2H-tetrazol-5-yl)phenyl morpholine-4-carboxylate;
6-(1-(4-(5-(difluoromethyl)-1,3,4-oxadiazol-2-yl)benzyl)-1H-1,2,3-triazol-4-yl)spiro[indoline-3,4′-piperidin]-2-one;
5-(1-(4-(5-(difluoromethyl)-1,3,4-oxadiazol-2-yl)benzyl)-1H-1,23-triazol-4-yl)-1,3-dimethyl-1,3-dihydro-2H-benzo(d)imidazol-2-imine;
3-(2-(4-(5-(difluoromethyl)-1,3,4-oxadiazol-2-yl)benzyl)-2H-tetrazol-5-yl)-4-fluoro-N,N-dimethylbenzenesulfonamide;
4-(2-(4-(5-(difluoromethyl)-1,3,4-oxadiazol-2-yl)benzyl)-2H-tetrazol-5-yl)-N1-methylbenzene-1,2-diamine;
N-(3-(1-(1-(4-(5-(difluoromethyl-1,3,4-oxadiazol-2-yl)-2-fluorophenyl)ethyl)-1H-1,2,3-triazol-4-yl)phenyl)morpholine-4-carboxamide;
6-(1-(4-(5-(difluoromethyl)-1,3,4-oxadiazol-2-yl)benzyl)-1H-1,2,3-triazol-4-yl)-1-methyl-1H-benzo[d]imidazol-2-amine;
5-(2-(4-(5-(difluoromethyl)-1,3,4-oxadiazol-2-yl)benzyl)-2H-tetrazol-5-yl)isoindolin-1-one;
5-(2-(4-(5-(difluoromethyl)-1,3,4-oxadiazol-2-yl)benzyl)-2H-tetrazol-5-yl)-1,3-dihydro-2H-benzo[d]imidazol-2-one;
2-(difluoromethyl)-5-(4-((4-(4-((4-(ethylsulfonyl)piperazin-1-yl)methyl)phenyl)-1H-1,2,3-triazol-1-methyl)-3,5-difluorophenyl)-1,3,4-oxadiazole;
1-(5-(5-(4-(5-(difluoromethyl)-1,3,4-oxadiazol-2-yl)benzyl)-1,3,4-oxadiazol-2-yl)pyridin-2-yl)-3-methylurea;
(S)-5-(1-(1-(5-(5-(difluoromethyl)-1,3,4-oxadiazol-2-yl)pyridin-2-yl)ethyl)-1H-1,2,3-triazol-4-yl)pyridin-2-amine;
tert-butyl (2-((3-(1-(4-(5-(difluoromethyl)-1,3,4-oxadiazol-2-yl)-2,6-difluorobenzyl)-1H-1,2,3-triazol-4-yl)phenyl)amino)-2-oxoethyl)carbamate;
7-(2-(4-(5-(difluoromethyl)-1,3,4-oxadiazol-2-yl)benzyl)-2H-tetrazol-5-yl)-2-methyl-3,4-dihydroisoquinolin-1 (2H)-one;
4-(6-(1-(4-(5-(difluoromethyl)-1,3,4-oxadiazol-2-yl)benzyl)-1H-1,2,3-triazol-4-yl)-1H-benzo[d]imidazol-2-yl)morpholine;
1-(3-(1-(4-(5-(difluoromethyl)-1,3,4-oxadiazol-2-yl)-2,6-difluorobenzyl)-1H-1,2,3-triazol-4-yl)phenyl)thiourea;
N-(5-(2-(4-(5-(difluoromethyl)-1,3,4-oxadiazol-2-yl)benzyl)-2H-tetrazol-5-yl)-2-(methylamino)phenyl)morpholine-4-carboxamide;
tert-butyl 5-(1-(4-(5-(difluoromethyl)-1,3,4-oxadiazol-2-yl)benzyl)-1H-1,2,3-triazol-4-yl)-2-oxospiro[indoline-3,3′-pyrrolidine]-1′-carboxylate;
6-(1-(4-(5-(difluoromethyl)-1,3,4-oxadiazol-2-yl)benzyl)-1H-1,2,3-triazol-4-yl)thieno[2,3-d]pyrimidin-4-amine;
N-(4-(1-(4-(5-(difluoromethyl)-1,3,4-oxadiazol-2-yl)-2,6-difluorobenzyl)-1H-1,2,3-triazol-4-yl)benzyl)-N-methyl-1-(pyridin-4-yl)methanamine;
3-(1-(4-(5-(difluoromethyl)-1,3,4-oxadiazol-2-yl)benzyl)-1H-1,2,3-triazol-4-yl)-N-ethylaniline;
2-(difluoromethyl)-5-(4-((5-(2-fluorophenyl)-2H-tetrazol-2-yl)methyl)phenyl)-1,3,4-oxadiazole;
(S)-2-(difluoromethyl)-5-(4-((5-(6-(3-methylpiperazin-1-yl)pyridin-3-yl)-2H-tetrazol-2-yl)methyl)phenyl)-1,3,4-oxadiazole;
N-(3-(1-(4-(5-(difluoromethyl)-1,3,4-oxadiazol-2-yl)-2,6-difluorobenzyl)-1H-1,2,3-triazol-4-yl)phenyl)-N-(furan-2-ylmethyl)acetamide;
N-(3-(1-(1-(4-(5-(difluoromethyl)-1,3,4-oxadiazol-2-yl)phenyl)propyl)-1H-1,2,3-triazol-4-yl)phenyl)morpholine-4-carboxamide;
5-(1-(1-(4-(5-(difluoromethyl)-1,3,4-oxadiazol-2-yl-2-fluorophenyl)ethyl)-1H-1,2,3-triazol-4-yl)pyridin-2-amine;
5-(1-(1-(4-(5-(difluoromethyl)-1,3,4-oxadiazol-2-yl)-3-fluorophenyl)ethyl)-1H-1,2,3-triazol-4-yl)pyridin-2-amine;
2-(difluoromethyl)-5-(2-((5-(thiophen-2-yl)-2H-tetrazol-2-yl)methyl)pyrimidin-5-yl)-1,3,4-oxadiazole;
2-(4-((5-(3-(1H-pyrazol-1-yl)phenyl)-2H-tetrazol-2-yl)methyl)phenyl)-5-(difluoromethyl)-1,3,4-oxadiazole;
N-(3-(1-(1-(4-(5-(difluoromethyl)-1,3,4-oxadiazol-2-yl)-3-fluorophenyl)ethyl) 1H-1,2,3-triazol-4-yl)phenyl)morpholine-4-carboxamide;
2-(difluoromethyl)-5-(4-((4-(2-(pyrrolidin-1-yl)-1H-benzo[d]imidazol-6-yl)-1H-1,2,3-triazol-1-yl)methyl)phenyl)-1,3,4-oxadiazole;
(4-(3-(4-(5-(difluoromethyl)-1,3,4-oxadiazol-2-yl)benzyl)-1,2,4-oxadiazol-5-yl)phenyl)methanamine;
3-(1-(4-(5-(difluoromethyl)-1,3,4-oxadiazol-2-yl)benzyl)-1H-1,2,3-triazol-4-yl)aniline;
5-(1-(1-(4-(5-(difluoromethyl)-1,3,4-oxadiazol-2-yl)phenyl)butyl)-1H-1,2,3-triazol-4-yl)pyridin-2-amine;
5-(1-(1-(4-(5-(difluoromethyl)-1,3,4-oxadiazol-2-yl)phenyl)propyl)-1H-1,2,3-triazol-4-yl)pyridin-2-amine;
6′-(2-(4-(5-(difluoromethyl)-1,3,4-oxadiazol-2-yl)benzyl)-2H-tetrazol-5-yl)-1′,4′-dihydro-3′H-spiro[cyclopentane-1,2′-quinoxalin]-3′-one;
4-(2-(4-(5-(difluoromethyl)-1,3,4-oxadiazol-2-yl)-2,6-difluorobenzyl)-2H-tetrazol-5-yl)-2-(morpholine-4-carboxamido)phenyl morpholine-4-carboxylate;
3-(1-(4-(5-(difluoromethyl)-1,3,4-oxadiazol-2-yl)benzyl)-1H-imidazol-4-yl)aniline;
5-(1-((6-(5-(difluoromethyl)-1,3,4-oxadiazol-2-yl)pyridazin-3-yl)methyl)-1H-1,2,3-triazol-4-yl)pyridin-2-amine;
N-(5-(2-(4-(5-(difluoromethyl)-1,3,4-oxadiazol-2-yl)benzyl)-2H-tetrazol-5-yl)pyridin-3-yl)morpholine-4-carboxamide;
5-(3-(2-(4-(5-(difluoromethyl)-1,3,4-oxadiazol-2-yl)benzyl)-2H-tetrazol-5-yl)phenyl)thiazol-2-amine;
N-(4-(2-(4-(5-(difluoromethyl)-1,3,4-oxadiazol-2-yl)-2,6-difluorobenzyl)-2H-tetrazol-5-yl)benzyl)-N-methyl-1-(pyridin-4-yl)methanamine;
5-(5-(4-(5-(difluoromethyl)-1,3,4-oxadiazol-2-yl)benzyl)isoxazol-3-yl)pyridin-2-amine;
6-(2-(4-(5-(difluoromethyl)-1,3,4-oxadiazol-2-yl)benzyl)-2H-tetrazol-5-yl)-2,3-dihydro-1H-inden-1-one;
2-(difluoromethyl)-5-(4-((5-(4-methoxyphenyl)-2H-tetrazol-2-yl)methyl)phenyl)-1,3,4-oxadiazole;
N-(3-(1-(1-(4-(5-(difluoromethyl)-1,3,4-oxadiazol-2-yl)phenyl)butyl)-1H-1,2,3-triazol-4-yl)phenyl)morpholine-4-carboxamide;
N-(4-(3-(4-(5-(difluoromethyl)-1,3,4-oxadiazol-2-yl)-2,6-difluorobenzyl)-1,2,4-oxadiazol-5-yl)benzyl)-2,2-difluoro-N-methylacetamide;
2-(4-((5-(benzo[b]thiophen-3-yl)-2H-tetrazol-2-yl)methyl)phenyl)-5-(difluoromethyl)-1,3,4-oxadiazole;
4-(2-(4-(5-(difluoromethyl)-1,3,4-oxadiazol-2-yl)benzyl)-2H-tetrazol-5-yl)-2,3-dihydro-1H-inden-1-one;
6′-(2-(4-(5-(difluoromethyl)-1,3,4-oxadiazol-2-yl)benzyl)-2H-tetrazol-5-yl)-1′,4′-dihydro-3′H-spiro[cyclohexane-1,2′-quinoxalin]-3′-one;
5-(2-(4-(5-(difluoromethyl)-1,3,4-oxadiazol-2-yl)benzyl)-2H-tetrazol-5-yl)-1-methyl-1,3-dihydro-2H-benzo[d]imidazol-2-one;
5-(1-(4-(5-(difluoromethyl)-1,3,4-oxadiazol-2-yl)benzyl)-1H-pyrazol-4-yl)pyridin-2-amine;
2-(difluoromethyl)-5-(4-((5-(6-(4-methylpiperazin-1-yl)pyridin-3-yl)-2H-tetrazol-2-yl)methyl)phenyl)-1,3,4-oxadiazole;
2-(difluoromethyl)-5 (4-((5-(4-(4-methylpiperazin-1-yl)phenyl)-2H-tetrazol-2-yl)methyl)phenyl)-1,3,4-oxadiazole;
2-(3,5-difluoro-4-((4-(4-((3-(trifluoromethyl)azetidin-1-yl)methyl)phenyl)-1H-1,2,3-triazol-1-yl)ethyl)phenyl-5-(difluoromethyl)-1,3,4-oxadiazole;
N-(4-(2-(4-(5-(difluoromethyl)-1,3,4-oxadiazol-2-yl)benzyl)-2H-tetrazol-5-yl)benzyl)-N-methyl-1-(pyridin-4-yl)methanamine;
tert-butyl 5-(1-(4-(5-(difluoromethyl)-1,3,4-oxadiazol-2-yl)benzyl)-1H-1,2,3-triazol-4-yl)-2-oxospiro[indoline-3,4′-piperidine]-1′-carboxylate;
2-(4-(1-(4-(5-(difluoromethyl)-1,3,4-oxadiazol-2-yl)benzyl)-1H-1,2,3-triazol-4-yl)phenyl)-1,1,3,3-tetramethylguanidine;
5-(5-(4-(5-(difluoromethyl)-1,3,4-oxadiazol-2-yl)benzyl)-1,3,4-oxadiazol-2-yl)pyridin-2-amine;
2-(difluoromethyl)-5-(4-((5-(2-(pyridin-4-yl)propan-2-yl)-2H-tetrazol-2-yl)methyl)phenyl)-1,3,4-oxadiazole;
2-(difluoromethyl)-5-(4-((5-(furan-2-yl)-2H-tetrazol-2-yl)methyl)phenyl)-1,3,4-oxadiazole;
5-(1-(1-(4-(5-(difluoromethyl)-1,3,4-oxadiazol-2-yl)phenyl)-2-phenylethyl)-1H-1,2,3-triazol-4-yl)pyridin-2-amine;
2-(4-((4-(1H-indazol-6-yl)-1H-1,2,3-triazol-1-yl)methyl)phenyl)-5-(difluoromethyl)-1,3,4-oxadiazole;
3-(1-(4-(5-(difluoromethyl)-1,3,4-oxadiazol-2-yl)benzyl)-1H-imidazol-4-yl)benzamide;
2-(difluoromethyl)-5-(4-((5-(3-fluoro-4-(piperazin-1-yl)phenyl)-2H-tetrazol-2-yl)methyl)phenyl)-1,3,4-oxadiazole;
5-(2-(4-(5-(difluoromethyl)-1,3,4-oxadiazol-2-yl)benzyl)-2H-tetrazol-5-yl)benzo[d]oxazol-2 (3H)-one;
3-(5-(4-(5-(difluoromethyl)-1,3,4-oxadiazol-2-yl)benzyl)-1,2,4-oxadiazol-3-yl)benzamide;
N-(3-(1-(4-(5-(difluoromethyl)-1,3,4-oxadiazol-2-yl)benzyl)-1H-pyrazol-4-yl)phenyl)morpholine-4-carboxamide;
N-(3-(3-(4-(5-(difluoromethyl)-1,3,4-oxadiazol-2-yl)benzyl)-1,2,4-oxadiazol-5-yl)phenyl)morpholine-4-carboxamide;
7-(2-(1-(4-(5-(difluoromethyl)-1,3,4-oxadiazol-2-yl)phenyl)ethyl)-2H-tetrazol-5-yl)-2-methyl-3,4-dihydroisoquinolin-1 (2H)-one;
(4-(2-(4-(5-(difluoromethyl)-1,3,4-oxadiazol-2-yl)benzyl)-2H-tetrazol-5-yl)phenyl)morpholino)methanone;
5-(1-(2-(4-chlorophenyl)-1-(4-(5-(difluoromethyl)-1,3,4-oxadiazol-2-yl)phenyl)ethyl)-1H-1,2,3-triazol-4-yl)pyridin-2-amine;
4-(2-(4-(5-(difluoromethyl)-1,3,4-oxadiazol-2-yl)benzyl)-2H-tetrazol-5-yl)-N-(1-methylpiperidin-4-yl)benzamide;
2-(difluoromethyl)-5-(4-((4-(2-methoxyphenyl)-1H-1,2,3-triazol-1-yl)methyl)phenyl)-1,3,4-oxadiazole;
2-(difluoromethyl)-5-(4-((4-phenyl-1H-1,2,3-triazol-1-yl)methyl)phenyl)-1,3,4-oxadiazole;
5-(1-(1-(4-(5-(difluoromethyl)-1,3,4-oxadiazol-2-yl)phenyl)pentyl)-1H-1,2,3-triazol-4-yl)pyridin-2-amine;
5-(1-(1-(4-(5-(difluoromethyl)-1,3,4-oxadiazol-2-yl)phenyl)-2-phenoxyethyl)-1H-1,2,3-triazol-4-yl)pyridin-2-amine;
8-(1-(4-(5-(difluoromethyl)-1,3,4-oxadiazol-2-yl)-2,6-difluorobenzyl)-1H-1,2,3-triazol-4-yl)-4-methyl-1,3,4,5-tetrahydro-2H-benzo[e][1,4]diazepin-2-one;
2-(difluoromethyl)-5-(4-((5-phenyl-1,3,4-thiadiazol-2-yl)methyl)phenyl)-1,3,4-oxadiazole;
N-(cyclopropylmethyl)-1-(4-(1-(4-(5-(difluoromethyl)-1,3,4-oxadiazol-2-yl)-2,6-difluorobenzyl)-1H-1,2,3-triazol-4-yl)benzoyl)piperidine-3-carboxamide;
tert-butyl 3-(3-(2-(4-(5-(difluoromethyl)-1,3,4-oxadiazol-2-yl)benzyl)-2H-tetrazol-5-yl)phenyl)-6,7-dihydropyrazolo[1,5-a]pyrazine-5 (4H)-carboxylate;
2-(difluoromethyl)-5-(4-((4-(6-fluoro-2-methylpyridin-3-yl)-1H-1,2,3-triazol-1-yl)methyl)phenyl)-1,3,4-oxadiazole;
5-(1-(2-cyclobutyl-1-(4-(5-(difluoromethyl)-1,3,4-oxadiazol-2-yl)phenyl)ethyl)-1H-1,2,3-triazol-4-yl)pyridin-2-amine;
5-(5-((4-(5-(difluoromethyl)-1,3,4-oxadiazol-2-yl)phenyl)difluoromethyl)-1,2,4-oxadiazol-3-yl)pyridin-2-amine;
N-(3-(1-(1-(4-(5-(difluoromethyl)-1,3,4-oxadiazol-2-yl)phenyl)phenyl)-1H-1,2,3-triazol-4-yl)phenyl)morpholine-4-carboxamide;
6 (1-(4-(5-(difluoromethyl)-1,3,4-oxadiazol-2-yl)benzyl)-1H-1,2,3-triazol-4-yl)-3,3-dimethylisoindolin-1-one;
2-(4-((5-([1,1′-biphenyl]-3-yl)-2H-tetrazol-2-yl)methyl)phenyl)-5-(difluoromethyl)-1,3,4-oxadiazole;
5-(3-(4-(5-(difluoromethyl)-1,3,4-oxadiazol-2-yl)benzyl)-1,2,4-oxadiazol-5-yl)pyridin-2-amine;
2-(difluoromethyl)-5-(4-((4-(3-fluorophenyl)-1H-1,2,3-triazol-1-yl)methyl)phenyl)-1,3,4-oxadiazole;
5-(2-(4-(5-(difluoromethyl)-1,3,4-oxadiazol-2-yl)benzyl)-2H-tetrazol-5-yl)-N,N-dimethylbenzo[d]oxazol-2-amine;
(S)-5-(1-(1-(4-(5-(difluoromethyl)-1,3,4-oxadiazol-2-yl)phenyl)butyl)-1H-1,2,3-thiazol-4-yl)pyridin-2-amine enantiomer;
2-(difluoromethyl)-5-(4-((5-(pyridin-2-ylmethyl)-2H-tetrazol-2-yl)methyl)phenyl)-1,3,4-oxadiazole;
5-(2-((5-(5-(difluoromethyl)-1,3,4-oxadiazol-2-yl)pyrimidin-2-yl)methyl)-2H-tetrazol-5-yl)-1-methyl-1H-benzo[d]imidazol-2-amine;
4-(5-(3-(2-(4-(5-(difluoromethyl)-1,3,4-oxadiazol-2-yl)benzyl)-2H-tetrazol-5-yl)phenyl)thiazol-2-yl)morpholine;
N-(4-(3-(4-(5-(difluoromethyl)-1,3,4-oxadiazol-2-yl)-2,6-difluorobenzyl)-1,2,4-oxadiazol-5-yl)benzyl)-N-methyl-1-(pyridin-4-yl)methanamine;
(S)-5-(1-(1-(4-(5-(difluoromethyl-1,3,4-oxadiazol-2-yl)phenyl)ethyl)-1H-1,2,3-triazol-4-yl)pyridin-2-amine;
2-(difluoromethyl)-5-(4-((5-(1-phenylcyclopropyl)-2H-tetrazol-2-yl)methyl)phenyl)-1,3,4-oxadiazole;
1-(4-(2-(4-(5-(difluoromethyl)-1,3,4-oxadiazol-2-yl)benzyl)-2H-tetrazol-5-yl)piperidin-1-yl)ethan-1-one;
N-(5-(2-(4-(5-(difluoromethyl-1,3,4-oxadiazol-2-yl)benzyl)-2H-tetrazol-5-yl)-2-(phenylthio)phenyl)morpholine-4-carboxamide;
N-(4-(3-(5-(difluoromethyl-1,3,4-oxadiazol-2-yl)benzyl)-1,2,4-oxadiazol-5-yl)benzyl)-2,2-difluoro-N-methylacetamide;
3-(2-(4-(5-(difluoromethyl)-1,3,4-oxadiazol-2-yl)benzyl)-2H-tetrazol-5-yl)benzoic acid;
2-(difluoromethyl)-5-(4-((5-(thiophen-2-yl)-2H-tetrazol-2-yl)methyl)phenyl)-1,3,4-oxadiazole;
3-(3-(4-(5-(difluoromethyl)-1,3,4-oxadiazol-2-yl)benzyl)-1,2,4-oxadiazol-5-yl)benzamide;
2-(4-((5-(2,4-dichlorophenyl)-2H-tetrazol-2-yl)methyl)phenyl)-5-(difluoromethyl)-1,3,4-oxadiazole;
N-(3-(1-((5-(5-(difluoromethyl)-1,3,4-oxadiazol-2-yl)pyridin-2-yl)methyl)-1H-imidazol-4-yl)phenyl)morpholine-4-carboxamide;
tert-butyl 5-(1-((5-(5-(difluoromethyl)-1,3,4-oxadiazol-2-yl)pyridin-2-yl)methyl)-1H-1,2,3-triazol-4-yl)-2-oxospiro[indoline-3,3′-pyrrolidine]-1′-carboxylate enantiomer A;
tert-butyl 5-(1-((5-(5-(difluoromethyl)-1,3,4-oxadiazol-2-yl)pyridin-2-yl)methyl)-1H-1,2,3-triazol-4-yl)-2-oxospiro[indoline-3,3′-pyrrolidine]-1′-carboxylate enantiomer B;
N-(3-(1-(4-(5-(difluoromethyl)-1,3,4-oxadiazol-2-yl)benzyl)-1H-imidazol-4-yl)phenyl)morpholine-4-carboxamide;
tert-butyl 7-(1-(4-(5-(difluoromethyl)-1,3,4-oxadiazol-2-yl)benzyl)-1H-1,2,3-triaz-4-yl)-3′-oxo-3′,4′-dihydro-1′H-spiro(piperidine-4,2′-quinoxaline)-1-carboxylate;
N-(4-(1-(4-(5-(difluoromethyl)-1,3,4-oxadiazol-2-yl)benzyl)-1H-pyrazol-4-yl)phenyl)-4,5-dihydro-1H-imidazol-2-amine;
N-(4-(1-((5-(5-(difluoromethyl)-1,3,4-oxadiazol-2-yl)pyridin-2-yl)methyl)-1H-pyrazol-4-yl)phenyl)-4,5-dihydro-1H-imidazol-2-amine;
7′-(1-(4-(5-(difluoromethyl)-1,3,4-oxadiazol-2-yl)benzyl)-1H-1,2,3-triazol-4-yl)-1′,4′-dihydro-3′H-spiro[piperidine-4,2′-quinoxalin]-3′-one;
tert-butyl 2-(3-(2-(4-(5-(difluoromethyl)-1,3,4-oxadiazol-2-yl)benzyl)-2H-tetrazol-5-yl)phenyl)-1,4,6,7-tetrahydro-5H-imidazo[4,5-c]pyridine-5-carboxylate;
5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one enantiomer A;
5-(1-((5-(5-(difluoromethyl)-1,3,4-oxadiazol-2-yl)pyridin-2-yl)methyl)-1H-1,2,3-triazol-4-yl)spiro[indoline-3,3′-pyrrolidin]-2-one enantiomer B;
S-(1-(4-(5-(difluoromethyl)-1,3,4-oxadiazol-2-yl)benzyl)-1H-1,2,3-triazol-4-yl)benzoic acid;
2-(difluoromethyl)-5-(6-((5-(3-(4,5,6,7-tetrahydro-1H-imidazo[4,5-c]pyridin-2-yl)phenyl)-2H-tetrazol-2-yl)methyl)pyridin-3-yl)-1,3,4-oxadiazole;
6′-(1-(4-(5-(difluoromethyl)-1,3,4-oxadiazol-2-yl)benzyl)-1H-1,2,3-triazol-4-yl)-1′,4′-dihydro-3′H-spiro[piperidine-4,2′-quinoxalin]-3′-one;
6-(2-(4-(5-(difluoromethyl)-1,3,4-oxadiazol-2-yl)benzyl)-2H-tetrazol-5-yl)quinazolin-2-amine;
tert-butyl 6′-(1-(4-(5-(difluoromethyl)-1,3,4-oxadiazol-2-yl)benzyl)-1H-1,2,3-triazol-4-yl)-3′-oxo-3′,4′-dihydro-1′H-spiro[piperidine-4,2′-quinoxaline]-1-carboxylate;
2-(difluoromethyl)-5-(4-((4-(imidazo[1,2-b]pyridazin-3-yl)-1H-1,2,3-triazol-1-yl)methyl)phenyl)-1,3,4-oxadiazole;
4-(1-(4-(5-(difluoromethyl)-1,3,4-oxadiazol-2-yl)benzyl)-1H-1,2,3-triazol-4-yl)-N,N-dimethylaniline;
N-(4-(1-(4-(5-(difluoromethyl)-1,3,4-oxadiazol-2-yl)benzyl)-1H-1,2,3-triazol-4-yl)benzyl)-methyl-1-(pyridin-4-yl)methanamine;
1-((1-(4-(5-(difluoromethyl)-1,3,4-oxadiazol-2-yl)-2,6-difluorobenzyl)-1H-1,2,3-triazol-4-yl)methyl)-1-ethyl-3-(2-methoxypyridin-3-yl)urea;
5-(5-((4-(5-(difluoromethyl)-1,3,4-oxadiazol-2-yl)-2,6-difluorophenyl)thio)-4-methyl-4H-1,2,4-triazol-3-yl)pyridin-2-amine;
5-(5-((4-(5-(difluoromethyl)-1,3,4-oxadiazol-2-yl)phenyl)thio)-4-methyl-4H-1,2,4-triazol-3-yl)pyridin-2-amine;
5-((4-(4-(4-(5-(difluoromethyl)-1,3,4-oxadiazol-2-yl)benzyl)-1H-1,2,3-triazol-1-yl)benzyl)amino)-2-methoxynicotinamide;
2-(difluoromethyl)-5-(4-((5-(pyrimidin-2-yl)-1H-tetrazol-1-yl)methyl)phenyl)-1,3,4-oxadiazole;
2-(4-((5-(benzo[b]thiophen-3-yl)-1H-tetrazol-1-yl)methyl)phenyl)-5-(difluoromethyl)-1,3,4-oxadiazole;
2-(4-((5-(3-(1H-pyrazol-1-yl)phenyl)-1H-tetrazol-1-yl)methyl)phenyl)-5-(difluoromethyl)-1,3,4-oxadiazole;
5-(1-((5-(5-(difluoromethyl)-1,3,4-oxadiazol-2-yl)pyrimidin-2-yl)methyl)-1H-tetrazol-5-yl)pyridin-2-amine;
5-(1-((5-(5-(difluoromethyl)-1,3,4-oxadiazol-2-yl)pyrimidin-2-yl)methyl)-1H-tetrazol-5-yl)-1-methyl-1H-benzo[d]imidazol-2-amine;
6-(1-((5-(5-(difluoromethyl)-1,3,4-oxadiazol-2-yl)pyridin-2-yl)methyl)-1H-imidazol-4-yl)isoindolin-1-one;
N-(3-(4-(6-aminopyridin-3-yl)-1H-1,2,3-triazol-1-yl)-3-(5-(5-(difluoromethyl)-1,3,4-oxadiazol-2-yl)pyridin-2-yl)propyl)methanesulfonamide;
6-(1-(4-(5-(difluoromethyl)-1,3,4-oxadiazol-2-yl)benzyl)-1H-imidazol-4-yl)isoindolin-1-one;
N-(3-(4-(6-aminopyridin-3-yl)-1H-1,2,3-triazol-1-yl)-3-(5-(5-(difluoromethyl)-1,3,4-oxadiazol-2-yl)pyridin-2-yl)propyl)-2,2-difluoroacetamide;
4-(1-(4-(5-(difluoromethyl)-1,3,4-oxadiazol-2-yl)-2,6-difluorobenzyl)-1H-1,2,3-triazol-4-yl)aniline;
3-(1-(4-(5-(difluoromethyl)-1,3,4-oxadiazol-2-yl)-2,6-difluorobenzyl)-1H-1,2,3-triazol-4-yl)aniline;
6-(1-((5-(5-(difluoromethyl)-1,3,4-oxadiazol-2-yl)pyrimidin-2-yl)methyl)-1H-tetrazol-5-yl)isoindolin-1-one;
2-(difluoromethyl)-5-(2-((5-(thiophen-2-yl)-1H-tetrazol-1-yl)methyl)pyrimidin-5-yl)-1,3,4-oxadiazole;
5-(1-(4-(5-(difluoromethyl)-1,3,4-oxadiazol-2-yl)benzyl)-1H-imidazol-4-yl)benzo[d]thiazol-2-amine and
5-(1-(1-(5-(5-(difluoromethyl-1,3,4-oxadiazol-2-yl)pyridin-2-yl)-2-(pyrrolidin-1-yl)ethyl)-1H-1,2,3-triazol-4-yl)pyridin-2-amine.

31. The compound according to claim 19, which is selected from:
N-[2-[4-(6-aminopyridin-3-yl)triazol-1-yl]-2-[4-[5-(difluoromethyl)-1,3,4-oxadiazol-2-yl]phenyl]ethyl]methanesulfonamide;
5-[1-[1-[4-[5-(difluoromethyl)-1,3,4-oxadiazol-2-yl]phenyl]-4-piperidin-1-ylbutyl]triazol-4-yl]pyridin-2-amine;
5-[1-[[5-[5-(difluoromethyl)-1,3,4-oxadiazol-2-yl]-3-fluoropyridin-2-yl]methyl]triazol-4-yl]pyridin-2-amine;
3-[1-[[5-[5-(difluoromethyl)-1,3,4-oxadiazol-2-yl]pyridin-2-yl]methyl]imidazol-4-yl]benzamide;
6-[1-[[4-[5-(difluoromethyl)-1,3,4-oxadiazol-2-yl]phenyl]methyl]imidazol-4-yl]-1,3-benzothiazol-2-amine;
6-[1-[[4-[5-(difluoromethyl)-1,3,4-oxadiazol-2-yl]pyridin-2-yl]methyl]imidazol-4-yl]-1,3-benzothiazol-2-amine;
5-[1-[[5-[5-(difluoromethyl)-1,3,4-oxadiazol-2-yl]pyridin-2-yl]methyl]imidazol-4-yl]-1,3-benzoxazol-2-amine;
5-[1-[[4-[5-(difluoromethyl)-1,3,4-oxadiazol-2-yl]phenyl]methyl]imidazol-4-yl]-1,3-benzoxazol-2-amine;
N-[(3S)-3-[4-(6-aminopyridin-3-yl)triazol-1-yl]-3-[4-[5-(difluoromethyl)-1,3,4-oxadiazol-2-yl]phenyl]propyl]methanesulfonamide;
N-[(3R)-3-[4-(6-aminopyridin-3-yl)triazol-1-yl]-3-[4-[5-(difluoromethyl)-1,3,4-oxadiazol-2-yl]phenyl]propyl]methanesulfonamide;
5-[1-[(1R)-1-[4-[5-(difluoromethyl)-1,3,4-oxadiazol-2-yl]phenyl]-2-pyrrolidin-1-ylethyl]triazol-4-yl]pyridin-2-amine;
5-[1-[(1S)-1-[4-[5-(difluoromethyl)-1,3,4-oxadiazol-2-yl]phenyl]-2-pyrrolidin-1-ylethyl]triazol-4-yl]pyridin-2-amine;
(2R)-2-[4-(6-aminopyridin-3-yl)triazol-1-yl]-2-[4-[5-(difluoromethyl)-1,3,4-oxadiazol-2-yl]phenyl]ethanol;
4-[4-[[4-[5-(difluoromethyl)-1,3,4-oxadiazol-2-yl]phenyl]methyl]triazol-1-yl]aniline;
N-[4-[4-[[4-[5-(difluoromethyl)-1,3,4-oxadiazol-2-yl]phenyl]methyl]triazol-1-yl]phenyl]-4,5-dihydro-1H-imidazol-2-amine;
7-[1-[[5-[5-(difluoromethyl)-1,3,4-oxadiazol-2-yl]pyridin-2-yl]methyl]triazol-4-yl]quinazolin-4-amine;
6-[1-[[5-[5-(difluoromethyl)-1,3,4-oxadiazol-2-ylpyridin-2-yl]]methyl]pyrazol-4-yl]-2,3-dihydroisoindol-1-one;
6-[1-[[4-[5-(difluoromethyl)-1,3,4-oxadiazol-2-yl]phenyl]methyl]pyrazol-4-yl]-2,3-dihydroisoindol-1-one;
5-[1-[[5-[5-(difluoromethyl)-1,3,4-oxadiazol-2-yl]pyridin-2-yl]methyl]pyrazol-4-yl]-1-methylbenzimidazol-2-amine;
5-[1-[[4-[5-(difluoromethyl)-1,3,4-oxadiazol-2-yl]phenyl]methyl]pyrazol-4-yl]-1-methylbenzimidazol-2-amine;
5-[1-[[5-[5-(difluoromethyl)-1,3,4-oxadiazol-2-yl]pyridin-2-yl]methyl]imidazol-4-yl]-1,3-benzothiazol-2-amine;
5-[1-[1-[[5-(difluoromethyl)-1,3,4-oxadiazol-2-yl]phenyl]-3-pyrrolidin-1-ylpropyl]triazol-4-yl]pyridin-2-amine;
5-[1-[[5-[5-(difluoromethyl)-1,3,4-oxadiazol-2-yl]pyridin-2-yl]methyl]triazol-4-yl]-3,3-dimethyl-1H-indol-2-one;
5-[1-[5-[5-(difluoromethyl)-1,3,4-oxadiazol-2-yl]pyridin-2-yl]methyl]triazol-4-yl)-1,3-dihydroindol-2-one;
6-[1-[[4-[5-(difluoromethyl)-1,3,4-oxadiazol-2-yl]phenyl]methyl]pyrazol-4-yl]-1,3-benzothiazol-2-amine;
6-[1-[[5-[5-(difluoromethyl)-1,3,4-oxadiazol-2-yl]pyridin-2-yl]methyl]pyrazol-4-yl]-1,3-benzothiazol-2-amine;
5-[1-[[5-[5-(difluoromethyl)-1,3,4-oxadiazol-2-yl]-pyridin-2-yl]methyl]imidazol-4-yl]-1-methylbenzimidazol-2-amine;
5-[1-[[4-[5-(difluoromethyl)-1,3,4-oxadiazol-2-yl]phenyl]methyl]imidazol-4-yl]-1-methylbenzimidazol-2-amine;
4-[5-[[4-[5-(difluoromethyl)-1,3,4-oxadiazol-2-yl]phenyl]methyl]-1,3-oxazol-2-yl]aniline;
5-[1-[[5-[5-(difluoromethyl)-1,3,4-oxadiazol-2-yl]pyridin-2-yl]methyl]pyrazol-4-yl]-1H-benzimidazol-2-amine;
5-[1-[[4-[5-(difluoromethyl)-1,3,4-oxadiazol-2-yl]phenyl]methyl]pyrazol-4-yl]-1H-benzimidazol-2-amine;
3-[1-[[4-[5-(difluoromethyl)-1,3,4-oxadiazol-2-yl]phenyl]methyl]pyrazol-4-yl]benzamide;
3-[1-[[5-[5-(difluoromethyl)-1,3,4-oxadiazol-2-yl]pyridin-2-yl]methyl]pyrazol-4-yl]benzamide;
4-[4-(6-aminopyridin-3-yl)triazol-1-yl]-4-[4-[5-(difluoromethyl)-1,3,4-oxadiazol-2-yl]phenyl]butan-1-ol;
N-[3-[1-[[5-[5-(difluoromethyl)-1,3,4-oxadiazol-2-yl]-3-fluoropyridin-2-yl]methyl]triazol-4-yl]phenyl]morpholine-4-carboxamide;
N-[3-[1-[[2-chloro-4-[5-(difluoromethyl)-1,3,4-oxadiazol-2-yl]phenyl]methyl]triazol-4-yl]phenyl]morpholine-4-carboxamide;
N-[3-[1-[[4-[5-(difluoromethyl)-1,3,4-oxadiazol-2-yl]-2,3-difluorophenyl]methyl]triazol-4-yl]phenyl]morpholine-4-carboxamide;
6-[1-[[4-[5-(difluoromethyl)-1,3,4-oxadiazol-2-yl]]-2-fluorophenyl]methyl]triazol-4-yl)-1,3-benzothiazol-2-amine;
6-[1-[[2-chloro-4-[5-(difluoromethyl)-1,3,4-oxadiazol-2-yl]phenyl]methyl]triazol-4-yl]-1,3-benzothiazol-2-amine;
6-[1-[[4-[5-(difluoromethyl)-1,3,4-oxadiazol-2-yl]-2,3-difluorophenyl]methyl]triazol-4-yl]-1,3-benzothiazol-2-amine;
6-[1-[[5-[5-(difluoromethyl)-1,3,4-oxadiazol-2-yl]-3-fluoropyridin-2-yl]methyl]triazol-4-yl]-1,3-benzothiazol-2-amine;
6-[1-[[4-[5-(difluoromethyl)-1,3,4-oxadiazol-2-yl]-2,5-difluorophenyl]methyl]triazol-4-yl]-1,3-benzothiazol-2-amine;
5-[1-[[2-chloro-4-[5-(difluoromethyl)-1,3,4-oxadiazol-2-yl]phenyl]methyl]triazol-4-yl]-1-methylbenzimidazol-2-amine;
5-[1-[[4-[5-(difluoromethyl)-1,3,4-oxadiazol-2-yl]-2,3-difluorophenyl]methyl]triazol-4-yl]-1-methylbenzimidazol-2-amine;
5-[1-[[4-[5-(difluoromethyl)-1,3,4-oxadiazol-2-yl]-3-fluorophenyl]methyl]triazol-4-yl]-1-methylbenzimidazol-2-amine;
6-[1-[[4-[5-(difluoromethyl)-1,3,4-oxadiazol-2-yl]-3-fluorophenyl]methyl]triazol-4-yl]-1,3-benzothiazol-2-amine;
5-[2-[[4-[5-(difluoromethyl)-1,3,4-oxadiazol-2-yl]-2-fluorophenyl]methyl]tetrazol-5-yl]-1-methylbenzimidazol-2-amine;
5-[1-[[5-[5-(difluoromethyl)-1,3,4-oxadiazol-2-yl]-3-fluoropyridin-2-yl]methyl]triazol-4-yl]-1-methylbenzimidazol-2-amine;
5-[1-[[4-[5-(difluoromethyl)-1,3,4-oxadiazol-2-yl]-2,5-difluorophenyl]methyl]triazol-4-yl]-1-methylbenzimidazol-2-amine;
6-[1-[dideutero-[4-[5-(difluoromethyl)-1,3,4-oxadiazol-2-yl]phenyl]methyl]triazol-4-yl]-1,3-benzothiazol-2-anine;
N-[3-[1-[dideuterio-[4-[5-(difluoromethyl)-1,3,4-oxadiazol-2-yl]phenyl]methyl]triazol-4-yl]phenyl]morpholine-4-carboxamide;
2-(difluoromethyl)-5-[5-fluoro-6-[[5-[3-(4,5,6,7-tetrahydro-1H-imidazo[4,5-c]pyridin-2-yl)phenyl]tetrazol-2-yl]methyl]pyridin-3-yl]-1,3,4-oxadiazole;
2-(difluoromethyl)-5-[3-fluoro-4-[[5-[3-(4,5,6,7-tetrahydro-1H-imidazo[4,5-c]pyridin-2-yl)phenyl]tetrazol-2-yl]methyl]phenyl]-1,3,4-oxadiazole;
2-(difluoromethyl)-5-[2,3-difluoro-4-[[5-[3-(4,5,6,7-tetrahydro-1H-imidazo[4,5-c]pyridin-2-yl)phenyl]tetrazol-2-yl]methyl]phenyl]-1,3,4-oxadiazole;
5-[1-[dideuterio-[4-[5-(difluoromethyl)-1,3,4-oxadiazol-2-yl]phenyl]methyl]triazol-4-yl]pyridin-2-amine;
N-[4-[1-[[4-[5-(difluoromethyl-1,3,4-oxadiazol-2-yl]-2-fluorophenyl]methyl]imidazol-4-yl]phenyl]-4,5-dihydro-1H-imidazol-2-amine;
N-[4-[1-[[2-chloro-4-[5-(difluoromethyl)-1,3,4-oxadiazol-2-yl]phenyl]methyl]imidazol-4-yl]phenyl]-4,5-dihydro-1H-imidazol-2-amine;
N-[4-[1-[[4-[5-(difluoromethyl)-1,3,4-oxadiazol-2-yl]-2,3-difluorophenyl]methyl]imidazol-4-yl]phenyl]-4,5-dihydro-1H-imidazol-2-amine;
N-[4-[1-[[4-[5-(difluoromethyl)-1,3,4-oxadiazol-2-yl]-3-fluorophenyl]methyl]imidazol-4-yl]phenyl]-4,5-dihydro-1H-imidazol-2-amine;
N-[4-[1-[[4-[5-(difluoromethyl)-1,3,4-oxadiazol-2-yl]-2,5-difluorophenyl]methyl]imidazol-4-yl]phenyl]-4,5-dihydro-1H-imidazol-2-amine;
5-[1-[dideuterio-[5-[5-(difluoromethyl)-1,3,4-oxadiazol-2-yl]pyridin-2-yl]methyl]triazol-4-yl]pyridin-2-amine;
N-[3-[1-[[4-[5-(difluoromethyl)-1,3,4-oxadiazol-2-yl]-2,5-difluorophenyl]methyl]triazol-4-yl]phenyl]morpholine-4-carboxamide;
2-(difluoromethyl)-5-[2-fluoro-4-[[5-[3-(4,5,6,7-tetrahydro-3H-imidazo[4,5-c]pyridin-2-yl)phenyl]tetrazol-2-yl]methyl]phenyl]-1,3,4-oxadiazole;
2-[3-chloro-4-[[5-[3-(4,5,6,7-tetrahydro-3H-imidazo[4,5-c]pyridin-2-yl)phenyl]tetrazol-2-yl]methyl]phenyl]-5-(difluoromethyl)-1,3,4-oxadiazole;
6-[5-[[4-[5-(difluoromethyl)-1,3,4-oxadiazol-2-yl]phenyl]methyl]-1,2-oxazol-3-yl]-1,3-benzothiazol-2-amine;
2-(difluoromethyl)-5-[2,5-difluoro-4-[[5-[3-(4,5,6,7-tetrahydro-1H-Imidazo[4,5-c]pyridin-2-yl)phenyl]tetrazol-2-yl]methyl]phenyl]-1,3,4-oxadiazole;
N-[4-[1-[dideuterio-[4-[5-(difluoromethyl)-1,3,4-oxadiazol-2-yl]phenyl]methyl]imidazol-4-yl]phenyl]-4,5-dihydro-1H-imidazol-2-amine;
N-[3-[1-[dideuterio-[5-[5-(difluoromethyl)-1,3,4-oxadiazol-2-yl]pyridin-2-yl]methyl]triazol-4-yl]phenyl]morpholine-4-carboxamide;
5-[2-[[5-[5-(difluoromethyl)-1,3,4-oxadiazol-2-yl]-3-fluoropyridin-2-yl]methyl]tetrazol-5-yl]-1-ethylbenzimidazol-2-amine;
5-[2-[[4-[5-(difluoromethyl)-1,3,4-oxadiazol-2-yl]-2,3-difluorophenyl]methyl]tetrazol-5-yl]-1-methylbenzimidazol-2-amine;
5-[2-[[4-[5-(difluoromethyl)-1,3,4-oxadiazol-2-yl]-2,5-difluorophenyl]methyl]tetrazol-5-yl]-1-methylbenzimidazol-2-amine;
5-[2-[[4-[5-(difluoromethyl)-1,3,4-oxadiazol-2-yl]-3-fluorophenyl]methyl]tetrazol-5-yl]-1-methylbenzimidazol-2-amine;
5-[2-[[2-chloro-4-[5-(difluoromethyl)-1,3,4-oxadiazol-2-yl]phenyl]methyl]tetrazol-5-yl]-1-methylbenzimidazol-2-amine;
4-[5-[4-[5-(difluoromethyl)-1,3,4-oxadiazol-2-yl]phenyl]methyl)-1,2,4-oxadiazol-3-yl]aniline;
6-[1-[dideuterio-[5-[5-(difluoromethyl)-1,3,4-oxadiazol-2-yl]pyridin-2-yl]methyl]triazol-4-yl]-1,3-benzothiazol-2-amine;
6-[4-[[4-[5-(difluoromethyl)-1,3,4-oxadiazol-2-yl]phenyl]methyl]triazol-1l-yl]-1,3-benzothiazol-2-amine;
5-[1-[[4-[5-(difluoromethyl)-1,3,4-oxadiazol-2-yl]-2-fluorophenyl]methyl]triazol-4-yl]-1-methylbenzimidazol-2-amine;
N-[4-[5-[[4-[5-(difluoromethyl)-1,3,4-oxadiazol-2-yl]phenyl]methyl]-1,2,4-oxadiazol-3-yl]phenyl]-4,5-dihydro-1H-imidazol-2-amine;
5-[1-[dideutero-[4-[5-(difluoromethyl)-1,3,4-oxadiazol-2-yl]-2,5-difluorophenyl]methyl]triazol-4-yl]pyridin-2-amine;
6-[1-[dideuterio-[4-[5-(difluoromethyl)-1,3,4-oxadiazol-2-yl]2-fluorophenyl]methyl]triazol-4-yl]-1,3-benzothiazol-2-amine;
N-(4-(5-(4-(5-(difluoromethyl)-1,3,4-oxadiazol-2-yl)-3-fluorobenzyl)-1,2,4-oxadiazol-3-yl)phenyl)-4,5-dihydro-1H-imidazol-2-amine;
6-(1-(4-(5-(difluoromethyl)-1,3,4-oxadiazol-2-yl)-2,3-difluorobenzyl)-1H-1,2,3-triazol-4-yl)thieno[2,3-d]pyrimidin-4-amine;
5-[1-[dideuterio-[4-[5-(difluoromethyl)-1,3,4-oxadiazol-2-yl]-2,3-difluorophenyl]methyl]triazol-4-yl]pyridin-2-amine;
6-(1-(4-(5-(difluoromethyl)-1,3,4-oxadiazol-2-yl)-2,6-difluorobenzyl)-1H-1,2,3-triazol-4-yl)thieno[2,3-d]pyrimidin-4-amine;
7-[1-({4-[5-(difluoromethyl)-1,3,4-oxadiazol-2-yl]-2-fluorophenyl}methyl)-1H-1,2,3-triazol-4-yl]quinazolin-4-amine;
6-(2-(4-(5-(difluoromethyl)-1,3,4-oxadiazol-2-yl)-2,6-difluorobenzyl)-2H-tetrazol-5-yl)-N-methylquinolin-2-amine;
6-[1-((4-[5-(difluoromethyl)-1,3,4-oxadiazol-2-yl]-2,3-difluorophenyl)methyl)-1H-1,2,3-triazol-4-yl]-N-methylquinazolin-2-amine;
6-[1-({4-[5-(difluoromethyl)-1,3,4-oxadiazol-2-yl]-2-fluorophenyl}methyl)-1H-1,2,3-triazol-4-yl]-N-methylquinazolin-2-amine;
6-[1-({4-[5-(difluoromethyl)-1,3,4-oxadiazol-2-yl]-2,5-difluorophenyl}methyl)-1H-1,2,3-triazol-4-yl]-N-methylquinazolin-2-amine;
6-[1-({4-[5-(difluoromethyl)-1,3,4-oxadiazol-2-yl]-2,6-difluorophenyl}methyl)-1H-1,2,3-triazol-4-yl]-N-methylquinazolin-2-amine;
6-[1-({4-[5-(difluoromethyl)-1,3,4-oxadiazol-2-yl]-3-fluorophenyl}methyl)-1H-1,2,3-triazol-4-yl]-N-methylquinazolin-2-amine;
6-[1-({4-[5-(difluoromethyl)-1,3,4-oxadiazol-2-yl]-2,5-difluorophenyl}methyl)-1H-1,2,3-triazol-4-yl]-N-ethylquinazolin-2-amine;
6-(1-(4-(5-(difluoromethyl)-1,3,4-oxadiazol-2-yl)-3-fluorobenzyl)-1H-1,2,3-triazol-4-yl)-N-ethylquinazolin-2-amine;
6-[2-[[4-[5-(difluoromethyl)-1,3,4-oxadiazol-2-yl]-2,5-difluorophenyl]methyl]tetrazol-5-yl]isoquinolin-1-amine;
6-[2-[[4-[5-(difluoromethyl)-1,3,4-oxadiazol-2-yl]-2-fluorophenyl]methyl]tetrazol-5-yl]isoquinolin-1-amine;
6-[2-[[4-[5-(difluoromethyl)-1,3,4-oxadiazol-2-yl]-2,5-difluorophenyl]methyl]tetrazol-5-yl]quinolin-3-amine;
6-(1-(4-(5-(difluoromethyl)-1,3,4-oxadiazol-2-yl)-2,6-difluorobenzyl)-1H-1,2,3-triazol-4-yl)-N,N-dimethylquinolin-2-amine;
6-[2-[[4-[5-(difluoromethyl)-1,3,4-oxadiazol-2-yl]-3-fluorophenyl]methyl]tetrazol-5-yl]quinolin-3-amine;
6-[2-[[4-[5-(difluoromethyl)-1,3,4-oxadiazol-2-yl]-2-fluorophenyl]methyl]tetrazol-5-yl]-N-methylquinolin-2-amine;
6-[1-({4-[5-(difluoromethyl)-1,3,4-oxadiazol-2-yl]-3-fluorophenyl}methyl)-1H-1,2,3-triazol-4-yl]-N,N-dimethylquinazolin-2-amine;
6-[2-[[4-[5-(difluoromethyl)-1,3,4-oxadiazol-2-yl]-3-fluorophenyl]methyl]tetrazol-5-yl]-N-methylquinolin-2-amine;
6-(1-(2-chloro-4-(5-(difluoromethyl)-1,3,4-oxadiazol-2-yl)benzyl)-1H-1,2,3-triazol-4-yl)isoquinolin-3-amine;
6-[1-({4-[5-(difluoromethyl)-1,3,4-oxadiazol-2-yl]-2-fluorophenyl}methyl)-1H-1,2,3-triazol-4-yl]isoquinolin-3-amine;
6-[1-({4-[5-(difluoromethyl)-1,3,4-oxadiazol-2-yl]-2,5-difluorophenyl}methyl)-1H-1,2,3-triazol-4-yl]isoquinolin-3-amine;
6-(2-(4-(5-(difluoromethyl)-1,3,4-oxadiazol-2-yl)-2,3-difluorobenzyl)-2H-tetrazol-5-yl)-N-methylquinolin-2-amine;
4-(5-(4-(5-(difluoromethyl)-1,3,4-oxadiazol-2-yl)-3-fluorobenzyl)-1,2,4-oxadiazol-3-yl)aniline;
6-(2-(4-(5-(difluoromethyl)-1,3,4-oxadiazol-2-yl)-2,5-difluorobenzyl)-2H-tetrazol-5-yl)-N-ethylquinolin-2-amine;
6-(2-(4-(5-(difluoromethyl)-1,3,4-oxadiazol-2-yl)-3-fluorobenzyl)-2H-tetrazol-5-yl)-N-ethylquinolin-2-amine;
5-(4-(4-(5-(difluoromethyl)-1,3,4-oxadiazol-2-yl)benzyl)-1H-1,2,3-triazol-1-yl)pyridin-2-amine;
5-[4-({4-[5-(difluoromethyl)-1,3,4-oxadiazol-2-yl]phenyl}methyl)-1H-1,2,3-triazol-1-yl]-1-methyl-1H-1,3-benzodiazol-2-amine;
6-[1-({4-[5-(difluoromethyl)-1,3,4-oxadiazol-2-yl)-2,5-difluorophenyl}methyl)-1H-1,2,3-triazol-4-yl]]thieno[2,3-d]pyrimidin-4-amine;
6-[1-({4-[5-(difluoromethyl)-1,3,4-oxadiazol-2-yl]-2-fluorophenyl}methyl)-1H-1,2,3-triazol-4-yl]thieno[2,3-d]pyrimidin-4-amine;
6-[1-({4-[5-(difluoromethyl)-1,3,4-oxadiazol-2-yl]-3-fluorophenyl}methyl)-1H-1,2,3-triazol-4-yl]thieno[2,3-d]pyrimidin-4-amine;
7-(1-(4-(5-(difluoromethyl)-1,3,4-oxadiazol-2-yl)-3-fluorobenzyl)-1H-1,2,3-triazol-4-yl)quinazolin-4-amine;
4-(5-(4-(5-(difluoromethyl)-1,3,4-oxadiazol-2-yl)-2-fluorobenzyl)-1,2,4-oxadiazol-3-yl)aniline;
N-(4-(5-(4-(5-(difluoromethyl)-1,3,4-oxadiazol-2-yl)-2-fluorobenzyl)-1,2,4-oxadiazol-3-yl)phenyl)-4,5-dihydro-1H-Imidazol-2-amine;
6-(2-(2-choro-4-(5-(difluoromethyl)-1,3,4-oxadiazol-2-yl)benzyl)-2H-tetrazol-5-yl)isoquinolin-1-amine;
6-(2-(4-(5-(difluoromethyl)-1,3,4-oxadiazol-2-yl)-2,5-difluorobenzyl)-2H-tetrazol-5-yl)quinazolin-2-amine;
6-(2-(4-(5-(difluoromethyl)-1,3,4-oxadiazol-2-yl)-2-fluorobenzyl)-2H-tetrazol-5-yl)quinazolin-2-amine;
6-(2-(2-chloro-4-(5-(difluoromethyl)-1,3,4-oxadiazol-2-yl)benzyl)-2H-tetrazol-5-yl)quinazolin-2-amine;
2-(3-Choro-4-((5-(isoquinolin-6-yl)-2H-tetrazol-2-yl)methyl)phenyl)-5-(difluoromethyl)-1,3,4-oxadiazole;
2-(difluoromethyl)-5-(3-fluoro-4-((5-(isoquinolin-6-yl)-2H-tetrazol-2-yl)methyl)phenyl)-1,3,4-oxadiazole;
2-(2,5-difluoro-4-((5-(isoquinolin-6-yl)-2H-tetrazol-2-yl)methyl)phenyl)-5-(difluoromethyl)-1,3,4-oxadiazole;
6-(2-(2-chloro-4-(5-(difluoromethyl)-1,3,4-oxadiazol-2-yl)benzyl)-2H-tetrazol-5-yl)quinolin-3-amine;
2-(3-chloro-4-((5-(isoquinolin-1-yl)-2H-tetrazol-2-yl)methyl)phenyl)-5-(difluoromethyl)-1,3,4-oxadiazole;
2-(difluoromethyl)-5-(3-fluoro-4-((5-(isoquinolin-1-yl)-2H-tetrazol-2-yl)methyl)phenyl)-1,3,4-oxadiazole;
2-(2,5-difluoro-4-((5-(isoquinolin-1-yl)-2H-tetrazol-2-yl)methyl)phenyl)-5-(difluoromethyl)-1,3,4-oxadiazole;
7-(1-(4-(5-(difluoromethyl)-1,3,4-oxadiazol-2-yl)-2,5-difluorobenzyl)-1H-1,2,3-triazol-4-yl)quinazolin-4-amine;
7-(1-(2-chloro-4-(5-(difluoromethyl)-1,3,4-oxadiazol-2-yl)benzyl)-1H-1,2,3-triazol-4-yl)quinazolin-4-amine;
2-(difluoromethyl)-5-[3-fluoro-4-[[5-(1-pyrazin-2-ylcyclopropyl)tetrazol-2-yl]methyl]phenyl]-1,3,4-oxadiazole;
2-(difluoromethyl)-5-[2-fluoro-4-[[5-(1-pyrazin-2-ylcyclopropyl)tetrazol-2-yl]methyl]phenyl]-1,3,4-oxadiazole;
2-(difluoromethyl)-5-[2,3-difluoro-4-[[5-(1-pyrazin-2-ylcyclopropyl)tetrazol-2-yl]methyl]phenyl]-1,3,4-oxadiazole;
2-(difluoromethyl)-5-[2,5-difluoro-4-[[5-(1-pyrazin-2-ylcyclopropyl)tetrazol-2-yl]methyl]phenyl]-1,3,4-oxadiazole;
2-(difluoromethyl)-5-[3,5-difluoro-4-[[5-(1-pyrazin-2-ylcyclopropyl)tetrazol-2-yl]methyl]phenyl]-1,3,4-oxadiazole;
2-[3-chloro-4-[[5-(1-pyrazin-2-ylcyclopropyl)tetrazol-2-yl]methyl]phenyl]-5-(difluoromethyl)-1,3,4-oxadiazole;
6-[2-[2-[[4-[5-(difluoromethyl)-1,3,4-oxadiazol-2-yl]-2-fluorophenyl]methyl]tetrazol-5-yl]propan-2-yl]pyridin-3-amine;
6-[2-[2-[[4-[5-(difluoromethyl)-1,3,4-oxadiazol-2-yl]-3-fluorophenyl]methyl]tetrazol-5-yl]propan-2-yl]pyridin-3-amine;
6-[2-[2-[[4-[5-(difluoromethyl)-1,3,4-oxadiazol-2-yl]-2,3-difluorophenyl]methyl]tetrazol-5-yl]propan-2-yl]pyridin-3-amine;
6-[2-[2-[[4-[5-(difluoromethyl)-1,3,4-oxadiazol-2-yl]2,5-difluorophenyl]methyl]tetrazol-5-yl]propan-2-yl]pyridin-3-amine;
6-[2-[2-[[4-[5-(difluoromethyl)-1,3,4-oxadiazol-2-yl]-2,6-difluorophenyl]methyl]tetrazol-5-yl]propen-2-yl]pyridin-3-amine;
6-[2-[2-[[2-chloro-4-[5-(difluoromethyl)-1,3,4-oxadiazol-2-yl]phenyl]methyl]tetrazol-5-yl]propan-2-yl]pyridin-3-amine;
2-[2-[2-[[4-[5-(difluoromethyl)-1,3,4-oxadiazol-2-yl]-2-fluorophenyl]methyl]tetrazol-5-yl]propan-2-yl]pyridin-4-amine;
2-[2-[2-[[4-[5-(difluoromethyl)-1,3,4-oxadiazol-2-yl]-3-fluorophenyl]methyl]tetrazol-5-yl]propan-2-yl]pyridin-4-amine;
2-[2-[2-[[4-[5-(difluoromethyl)-1,3,4-oxadiazol-2-yl]-2,3-difluorophenyl]methyl]tetrazol-5-yl]propan-2-yl]pyridin-4-amine;
2-[2-[2-[[4-[5-(difluoromethyl)-1,3,4-oxadiazol-2-yl]-2,5-difluorophenyl]methyl]tetrazol-5-yl]propan-2-yl]pyridin-4-amine;
2-[2-[2-[[4-[5-(difluoromethyl)-1,3,4-oxadiazol-2-yl]-2,6-difluorophenyl]methyl]tetrazol-5-yl]propan-2-yl]pyridin-4-amine;
2-[2-[2-[[2-chloro-4-[5-(difluoromethyl)-1,3,4-oxadiazol-2-yl]phenyl]methyl]tetrazol-5-yl]propan-2-yl]pyridin-4-amine;
2-[2-[[4-[5-(difluoromethyl)-1,3,4-oxadiazol-2-yl]-2-fluorophenyl]methyl]tetrazol-5-yl]pyrimidin-5-amine;
2-[2-[[4-[5-(difluoromethyl)-1,3,4-oxadiazol-2-yl]-3-fluorophenyl]methyl]tetrazol-5-yl]pyrimidin-5-amine;
2-[2-[[4-[5-(difluoromethyl)-1,3,4-oxadiazol-2-yl]-2,3-difluorophenyl]methyl]tetrazol-5-yl]pyrimidin-5-amine;
2-[2-[[4-[5-(difluoromethyl)-1,3,4-oxadiazol-2-yl]-2,5-difluorophenyl]methyl]tetrazol-5-yl]pyrimidin-5-amine;
2-[2-[[4-[5-(difluoromethyl)-1,3,4-oxadiazol-2-yl]-2,6-difluorophenyl]methyl]tetrazol-5-yl]pyrimidin-5-amine;
2-[2-[[2-chloro-4-[5-(difluoromethyl)-1,3,4-oxadiazol-2-yl]phenyl]methyl]tetrazol-5-yl]pyrimidin-5-amine;
6-[2-[[4-[5-(difluoromethyl)-1,3,4-oxadiazol-2-yl]-2,3-difluorophenyl]methyl]tetrazol-5-yl]isoquinolin-1-amine;
8-[2-[[4-[5-(difluoromethyl)-1,3,4-oxadiazol-2-yl]-2,6-difluorophenyl]methyl]tetrazol-5-yl]isoquinolin-1-amine;
6-[2-[[4-[5-(difluoromethyl)-1,3,4-oxadiazol-2-yl]-2,6-difluorophenyl]methyl]tetrazol-5-yl]quinolin-3-amine;
6-[2-[[4-[5-(difluoromethyl)-1,3,4-oxadiazol-2-yl]-2,3-fluorophenyl]methyl]tetrazol-5-yl]quinolin-3-amine;
6-[2-[[4-[5-(difluoromethyl)-1,3,4-oxadiazol-2-yl]-3-fluorophenyl]methyl]tetrazol-5-yl]quinazolin-2-amine;
6-[2-[[4-[5-(difluoromethyl)-1,3,4-oxadiazol-2-yl]-2,3-difluorophenyl]methyl]tetrazol-5-yl]quinazolin-2-amine;
6-[2-[[4-[5-(difluoromethyl)-1,3,4-oxadiazol-2-yl]-2,6-difluorophenyl]methyl]tetrazol-5-yl]quinazolin-2-amine;
2-(difluoromethyl)-5-[2-fluoro-4-[(5-isoquinolin-6-yltetrazol-2-yl)methyl]phenyl]-1,3,4-oxadiazole;
2-[2,3-difluoro-4-[(5-isoquinolin-6-yltetrazol-2-yl)methyl]phenyl]-5-(difluoromethyl)-1,3,4-oxadiazole;
2-[3,5-difluoro-4-((5-isoquinolin-6-yltetrazol-2-yl)methyl]phenyl)-5-(difluoromethyl)-1,3,4-oxadiazole;
2-(difluoromethyl)-5-[2-fluoro-4-[(5-isoquinolin-1-yltetrazol-2-yl)methyl]phenyl]-1,3,4-oxadiazole;
2-[2,3-difluoro-4[(5-isoquinolin-1-yltetrazol-2-yl)methyl]phenyl]-5-(difluoromethyl)-1,3,4-oxadiazole;
2-[3,5-difluoro-4-[(5-isoquinolin-1-yltetrazol-2-yl)methyl]phenyl]-5-(difluoromethyl)-1,3,4-oxadiazole;
6-[2-[[2-chloro-4-[5-(difluoromethyl)-1,3,4-oxadiazol-2-yl]phenyl]methyl]tetrazol-5-yl]-N-methylquinolin-2-amine;
6-[2-[[4-[5-(difluoromethyl)-1,3,4-oxadiazol-2-yl]-2-fluorophenyl]methyl]tetrad-5-yl]-N-ethylquinolin-2-amine;
6-[2-[[4-[5-(difluoromethyl)-1,3,4-oxadiazol-2-yl]-2,3-difluorophenyl]methyl]tetrazol-5-yl]-N-ethylquinolin-2-amine;
6-[2-[[4-[5-(difluoromethyl)-1,3,4-oxadiazol-2-yl]-2,6-difluorophenyl]methyl]tetrazol-5-yl]-N-ethylquinolin-2-amine;
6-[2-[[2-chloro-4-[5-(difluoromethyl)-1,3,4-oxadiazol-2-yl]phenyl]methyl]tetrazol-5-yl]-N-ethylquinolin-2-amine;
6-1[(1R)-1-[4-[5-(difluoromethyl)-1,3,4-oxadiazol-2-yl]2-fluorophenyl]triazol-4-yl]-1,3-benzothiazol-2-amine;
6-[1-[(1R)-1-[2-chloro-4-[5-(difluoromethyl)-1,3,4-oxadiazol-2-yl]phenyl]ethyl]triazol-4-yl]-1,3-benzothiazol-2-amine;
6-[1-[(1R)-1-[4-[5-(difluoromethyl)-1,3,4-oxadiazol-2-yl]-3-fluorophenyl]ethyl]triazol-4-yl]-1,3-benzothiazol-2-amine;
6-[1-[(1R)-1-[4-[5-(difluoromethyl)-1,3,4-oxadiazol-2-yl]-2,3-fluorophenyl]ethyl]triazol-4-yl]-1,3-benzothiazol-2-amine;
6-[1-[(1R)-1-[4-[5-(difluoromethyl)-1,3,4-oxadiazol-2-yl]-2,5-difluorophenyl]ethyl]triazol-4-yl]-1,3-benzothiazol-2-amine;
6-[1-[(1R)-1-[4-[5-(difluoromethyl)-1,3,4-oxadiazol-2-yl]-2,6-difluorophenyl]ethyl]triazol-4-yl]-1,3-benzothiazol-2-amine;
6-[2-[[4-[5-(difluoromethyl)-1,3,4-oxadiazol-2-yl]-2,5-difluorophenyl]methyl]tetrazol-5-yl]-N-methylquinazolin-2-amine;
6-[2-[[4-[5-(difluoromethyl)-1,3,4-oxadiazol-2-yl]-3-fluorophenyl]methyl]tetrazol-5-yl]-N-methylquinazolin-2-amine;
6-[2-[[4-[5-(difluoromethyl)-1,3,4-oxadiazol-2-yl]-2-fluorophenyl]methyl]tetrazol-5-yl]-N-methylquinazolin-2-amine;
6-[2-[[4-[5-(difluoromethyl)-1,3,4-oxadiazol-2-yl]-2,5-difluorophenyl]methyl]tetrazol-5-yl]-N,N-dimethylquinazolin-2-amine;
6-[2-[[4-[5-(difluoromethyl)-1,3,4-oxadiazol-2-yl]-3-fluorophenyl]methyl]tetrazol-5-yl]-N,N-dimethylquinazolin-2-amine;
6-[2-[[4-[5-(difluoromethyl)-1,3,4-oxadiazol-2-yl]-2-fluorophenyl]methyl]tetrazol-5-yl]-N,N-dimethylquinazolin-2-amine;
6-[1-[[2-chloro-4-[5-(difluoromethyl)-1,3,4-oxadiazol-2-yl]phenyl]methyl]triazol-4-yl]-N-methylquinazolin-2-amine;
6-[1-[[4-[5-(difluoromethyl)-1,3,4-oxadiazol-2-yl]-2,3-difluorophenyl]methyl]triazol-4-yl]-N-ethylquinazolin-2-amine;
6-[1-[[4-[5-(difluoromethyl)-1,3,4-oxadiazol-2-yl]-2,6-difluorophenyl]methyl]triazol-4-yl]-N-ethylquinazolin-2-amine;
6-[1-[[2-chloro-4-[5-(difluoromethyl)-1,3,4-oxadiazol-2-yl]phenyl]methyl]triazol-4-yl]-N-ethylquinazolin-2-amine;
6-[1-[[4-[5-(difluoromethyl)-1,3,4-oxadiazol-2-yl]-2,5-difluorophenyl]methyl]triazol-4-yl]-N,N-dimethylquinazolin-2-amine;
6-[1-[[4-[5-(difluoromethyl)-1,3,4-oxadiazol-2-yl]-2,3-difluorophenyl]methyl]triazol-4-yl]-N,N-dimethylquinazolin-2-amine;
6-[1-[[4-[5-(difluoromethyl)-1,3,4-oxadiazol-2-yl]-2-fluorophenyl]methyl]triazol-4-yl]-N,N-dimethylquinazolin-2-amine;
6-[1-[[2-chloro-4-[5-(difluoromethyl)-1,3,4-oxadiazol-2-yl]phenyl]methyl]triazol-4-yl]-N,N-dimethylquinazolin-2-amine;
6-[1-[[4-[5-(difluoromethyl)-1,3,4-oxadiazol-2-yl]2,3-difluorophenyl]methyl]triazol-4-yl]isoquinolin-3-amine;
6-[1-[[4-[5-(difluoromethyl)-1,3,4-oxadiazol-2-yl]-2,6-difluorophenyl]methyl]triazol-4-yl]quinolin-3-amine;
6-[1-[[4-[5-(difluoromethyl)-1,3,4-oxadiazol-2-yl]-3-fluorophenyl]methyl]triazol-4-yl]isoquinolin-3-amine;
6-[1-[[2-chloro-4-[5-(difluoromethyl)-1,3,4-oxadiazol-2-yl]phenyl]methyl]triazol-4-yl]thieno[2,3-d]pyrimidin-4-amine;
7-[1-[[4-[5-(difluoromethyl)-1,3,4-oxadiazol-2-yl]-2,3-difluorophenyl]methyl]triazol-4-yl]quinazolin-4-amine;
7-[1-[[4-[5-(difluoromethyl)-1,3,4-oxadiazol-2-yl]-2,6-difluorophenyl]methyl]triazol-4-yl]quinazolin-4-amine;
6-(2-(4-(5-(difluoromethyl)-1,3,4-oxadiazol-2-yl)-2,5-difluorobenzyl)-2H-tetrazol-5-yl)-N-methylquinolin-2-amine;
5-[1-[dideuterio-[4-[5-(difluoromethyl)-1,3,4-oxadiazol-2-yl]-3-fluorophenyl]methyl]triazol-4-yl]pyridin-2-amine;
5-[1-[dideuterio-[4-[5-(difluoromethyl)-1,3,4-oxadiazol-2-yl]-2-fluorophenyl]methyl]triazol-4-yl]pyridin-2-amine;
6-[1-[dideuterio-[4-[5-(difluoromethyl)-1,3,4-oxadiazol-2-yl]-3-fluorophenyl]methyl]triazol-4-yl]-1,3-benzothiazol-2-amine;
6-[1-[dideuterio-[4-[5-(difluoromethyl)-1,3,4-oxadiazol-2-yl]-2,5-difluorophenyl]methyl]triazol-4-yl]-1,3-benzothiazol-2-amine;
6-[1-[dideuterio-[4-[5-(difluoromethyl)-1,3,4-oxadiazol-2-yl]-2,3-difluorophenyl]methyl]triazol-4-yl]-1,3-benzothiazol-2-amine;
5-[1-[dideuterio-[4-[5-(difluoromethyl)-1,3,4-oxadiazol-2-yl]-3-fluorophenyl]methyl]triazol-4-yl]-1-methylbenzimidazol-2-amine;
5-[1-[dideuterio-[4-[5-(difluoromethyl)-1,3,4-oxadiazol-2-yl]-2-fluorophenyl]methyl]triazol-4-yl]-1-methylbenzimidazol-2-amine;
5-[1-[dideuterio-[4-[5-(difluoromethyl)-1,3,4-oxadiazol-2-yl]-2,5-difluorophenyl]methyl]triazol-4-yl]-1-methylbenzimidazol-2-amine;
5-[1-[dideuterio-[4-[5-(difluoromethyl)-1,3,4-oxadiazol-2-yl]2,3-difluorophenyl]methyl]triazol-4-yl]-1-methylbenzimidazol-2-amine;
6-[5-[[4-[5-(difluoromethyl)-1,3,4-oxadiazol-2-yl]phenyl]methyl]-1,2,4-oxadiazol-3-yl]-1,3-benzothiazol-2-amine;
6-[2-[[4-[5-(difluoromethyl)-1,3,4-oxadiazol-2-yl]-3-fluorophenyl]methyl]tetrazol-5-yl]isoquinolin-1-amine;
6-[2-[[4-[5-(difluoromethyl)-1,3,4-oxadiazol-2-yl]-2-fluorophenyl]methyl]tetrazol-5-yl]quinolin-3-amine;
6-[2-[[4-[5-(difluoromethyl)-1,3,4-oxadiazol-2-yl]-2,5-difluorophenyl]methyl]tetrazol-5-yl]-N-methylquinolin-2-amine;
6-[2-[[4-[5-(difluoromethyl)-1,3,4-oxadiazol-2-yl]-2,5-difluorophenyl]methyl]tetrazol-5-yl]isoquinolin-3-amine;
6-[2-[[4-[5-(difluoromethyl)-1,3,4-oxadiazol-2-yl]-2-fluorophenyl]methyl]tetrazol-5-yl]isoquinolin-3-amine;
6-[2-[[4-[5-(difluoromethyl)-1,3,4-oxadiazol-2-yl]-3-fluorophenyl]methyl]tetrazol-5-yl]isoquinolin-3-amine;
7-[2-[[4-[5-(difluoromethyl)-1,3,4-oxadiazol-2-yl]-2,5-difluorophenyl]methyl]tetrazol-5-yl]quinazolin-4-amine;
7-[2-[[4-[5-(difluoromethyl)-1,3,4-oxadiazol-2-yl]-2-fluorophenyl]methyl]tetrazol-5-yl]quinazolin-4-amine;
7-[2-[[4-[5-(difluoromethyl)-1,3,4-oxadiazol-2-yl]-3-fluorophenyl]methyl]tetrazol-5-yl]quinazolin-4-amine;
6-[2-[[4-[5-(difluoromethyl)-1,3,4-oxadiazol-2-yl]-3-fluorophenyl]methyl]tetrazol-5-yl]thieno[2,3-d]pyrimidin-4-amine;
6-[2-[[4-[5-(difluoromethyl)-1,3,4-oxadiazol-2-yl]-2,5-difluorophenyl]methyl]tetrazol-5-yl]thieno[2,3-d]pyrimidin-4-amine and
6-[2-[[4-[5-(difluoromethyl)-1,3,4-oxadiazol-2-yl]-2-fluorophenyl]methyl]tetrazol-5-yl]thieno[2,3-d]pyrimidin-4-amine.

32. The compound according to claim 19, in combination with a drug selected from the group consisting of proteasome inhibitors; immunochemical inhibitors; steroids; bromodomain inhibitors; epigenetic drugs; traditional chemotherapeutic agents, including cisplatin and taxol; proteasome inhibitors, including bortezomib; kinase inhibitors, including JAK family; and CTLA4, PD1 or PDL1 checkpoint inhibitors including nivolumab, pemprolizumab, pidilizumab, BMS-938559, atezolizumab, avelumab, ipilimumab and tremelimumab.

33. A method of treating one or more HDAC-mediated diseases selected from the group consisting of chemotherapy-related cognitive impairment (CRCI), graft rejection, GVHD, myositis, diseases associated with abnormal lymphocyte functions, multiple myeloma, non-Hodgkin lymphoma, peripheral neuropathies, autoimmune diseases, inflammatory diseases, cancer and neurodegenerative diseases, and ocular diseases in a subject in need thereof, comprising administration of a therapeutically effective amount of the compound according to claim 19, alone or in combination with one or more pharmaceutically acceptable excipients.

34. A pharmaceutical composition comprising a therapeutically effective quantity of at least one compound according to claim 19, or pharmaceutically acceptable salt, isomer or prodrug thereof, together with at least one pharmaceutically acceptable excipient.

35. The pharmaceutical composition according to claim 34, which is suitable to be administered by an enteral route, parenteral route, oral route, topical route, or inhalatory route.

36. The pharmaceutical composition according to claim 34, which is in the form of a liquid or a solid.

37. The pharmaceutical composition according to claim 36, which is in the form of a capsule, tablet, coated tablet, powder, granule, cream or ointment.
